(19) United States
(12) Patent Application Publication
(10) Pub. No.: US 2024/0132493 A1
(43) Pub. Date: Apr. 25, 2024

(54) IMIDAZOPYRIDINE DERIVATIVES AS STING AGONISTS

(71) Applicant: Fulgent Genetics, Inc., El Monte, CA (US)

(72) Inventors: Ming HSIEH, Pasadena, CA (US); Jingcun ZANG, Beijing (CN); Chunlin TAO, Carlsbad, CA (US)

(21) Appl. No.: 18/517,300

(22) Filed: Nov. 22, 2023

Related U.S. Application Data
(63) Continuation of application No. PCT/CN2021/095496, filed on May 24, 2021.

(51) Int. Cl. C07D 471/04; A61K 39/39; A61P 35/00

(52) U.S. Cl. CPC C07D 471/04; A61K 39/39; A61P 35/00

(57) ABSTRACT

Described herein, inter alia, are imidazopyridine derivatives (I), pharmaceutically acceptable salts and tautomers thereof, compounds, combinations and medicaments containing said compounds and processes for their preparation. In embodiments, the imidazopyridine derivatives can be used as regulators of a stimulator of interferon genes (STING) and a related signal path thereof, and can effectively treat and/or relieve multiple types of diseases, including but not limited to malignant tumors, inflammations, autoimmune diseases, infectious diseases and as vaccine adjuvants.

### CROSS REFERENCE

This application is a continuation of International Application No. PCT/CN2021/095496, filed May 24, 2021, which is herein incorporated by reference in its entirety.

### BACKGROUND

Innate immunity is a critical component of host defense against various pathogens, such as viruses, bacteria, fungi and parasites.

STING (Stimulator of Interferon Genes) is an endoplasmic reticulum adaptor that facilitates innate immune signaling (Ishikawa, H., et al., Nature 2008, 455(7213):674-678) and was described as a transmembrane component of the endoplasmic reticulum (ER) essential for the production of type I IFN in fibroblasts, macrophages and dendritic cells (DCs) in response to cytoplasmic double-stranded DNA (dsDNA) as well as select DNA viruses and intracellular bacteria (a) Ishikawa, H., et al., Nature. 2008; 455(7213): 674-678; b) Ishikawa, H., et al., Nature. 2009; 461(7265):788-92).

Recent reports have also indicated that potent activators of the STING pathway may also include self-DNA that has leaked from the nucleus of the host cell, perhaps following cell division or as a consequence of DNA damage (Ahn, J., Curr Opin Immunol. 2014 Dec.; 31: 121-6). It has demonstrated that STING plays a key role in antitumor immune response ((a) Woo S. R., et al. Immunity. 2014, 41:830-842; b). Deng, L., et al. Immunity. 2014, 41: 843-852; c), Liu X., et al, Nat. Med., 21 (2015), 1209-1215). STING-dependent DNA detection was also found to trigger anticancer immunity. Importantly, activation of the STING pathway was correlated to the induction of a spontaneous antitumor T cell response involving the expression of Type I interferon (IFN) genes (a) Barber. G., Nat Rev Immunol. 2015 Dec.; 15(12):760-70; b) Chen Q., et al., Nat. Immunol. 2016; 17(10): 1142-9).

Moreover, an optimal combination of STING agonists with other treatments, including irradiation, chemotherapy, or blockade of immune-system checkpoints, may achieve a good clinical outcome (a) Li, T., et al, Sci. Rep. 2016, 6, 19049; b) Fu, J., et al, Sci. Transl. Med. 2015, 7(283); 283ra252; c) Deng, L., et al, Immunity 2014; 41: 843-852). An increasing amount of evidence indicates that intratumoral STING agonists are promising cancer therapeutic agents (a) Ager, C., et al., Cancer Immun. Res., 2017, 5(8), 676-684; b) Su, T., et al., Theranostics, 2019, 9(25) 7759-7771) and it is of high importance to study high affinity synthetic STING agonists as pontential anticancer therapeutic agents (Ramanjulu, J., et al., Nature (2018), 564(7736), 439-443).

In addition, cyclic dinucleotide STING agonists may comprise a novel class of vaccine adjuvants capable of inducing cellular immune responses and protective efficacy against intracellular pathogens (a) Hiroyasu, I., et al. Virology, 2019, 531, 233-239; b) Dey Ruchi Jain, D., et al., The Journal of infectious diseases, 2020, 221 (7), 1048-1056; c) Wang, J. et al., Science, 2020, 367, 869-880).

There remains a need for new immunotherapies for the treatment of diseases, cancer in particular.

### SUMMARY

The invention provides imidazopyridine derivatives as STING agonists, pharmaceutically acceptable salts thereof, pharmaceutical compositions thereof, and combinations thereof, which are useful for the treatment of diseases, cancer in particular. The invention further provides methods of treating, preventing, or ameliorating cancer for a subject in need of an effective amount of a compound of the invention.

In an aspect is provided a compound having the Formula:

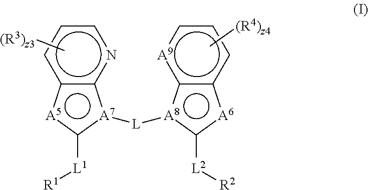

wherein:
R¹ is independently R¹⁰-substituted or unsubstituted C₁-C₂₀ saturated alkyl (e.g., C₁-C₈, C₁-C₆, C₁-C₄, or C₁-C₂), R¹⁰-substituted or unsubstituted C₂-C₂₀ alkenyl (e.g., C₂-C₈, C₂-C₆, C₂-C₄, or C₂), R¹⁰-substituted or unsubstituted C₂-C₂₀ alkynyl (e.g., C₂-C₈, C₂-C₆, C₂-C₄, or C₂), R¹⁰-substituted or unsubstituted C₃-C₁₀ cycloalkyl (e.g., C₃-C₈, C₃-C₆, C₄-C₆, or C₅-C₆), R¹⁰-substituted or unsubstituted 4-10 membered heterocycloalkyl (e.g., 4 to 8 membered, 4 to 6 membered, 4 to 5 membered, or 5 to 6 membered), R¹⁰-substituted or unsubstituted phenyl, R¹⁰-substituted or unsubstituted 6-10 membered aryl (e.g., C₆-C₁₀ or phenyl), or R¹⁰-substituted or unsubstituted 5-10 membered heteroaryl (e.g., 5 to 10 membered, 5 to 9 membered, or 5 to 6 membered);
wherein said R¹⁰-substituted C₁-C₂₀ saturated alkyl, R¹⁰-substituted C₂-C₂₀ alkenyl, R¹⁰-substituted C₂-C₂₀ alkynyl, R¹⁰-substituted C₃-C₁₀ cycloalkyl, R¹⁰-substituted phenyl, R¹⁰-substituted 6-10 membered aryl, R¹⁰-substituted 4-10 membered heterocycloalkyl, and R¹⁰-substituted 5-10 membered heteroaryl is substituted with 1 to 4 R¹⁰ substituents;
each R¹⁰ substituent is independently halogen, —CN, —CF₃, —OR^{10B}, —OC(O)R^{10B}, —OC(O)NR^{10A}R^{10B}, —C(O)—OR^{10B}, —C(O)NR^{10A}R^{10B}, —SOR^{10B}, —SO₂R^{10B}, —SO₂NR^{10A}R^{10B}, —O—P(O)(OR^{10A})₂, —NR^{10A}R^{10B}, —NR^{10A}C(O)R^{10B}, —NR^{10A}SOR^{10B}, —NR^{10A}C(O)OR^{10B}, —NR^{10A}SO₂R^{10B}, —C(O)R^{10B}, unsubstituted C₁-C₆ saturated alkyl, unsubstituted C₂-C₆ alkenyl, unsubstituted C₂-C₆ alkynyl, halo-substituted (C₁-C₄ alkyl), hydroxy-substituted (C₁-C₄ alkyl), (unsubstituted C₁-C₄ alkyl)-O—P(O)(OH)₂, (unsubstituted C₁-C₄ alkyl)-O—P(O)(OR^{10A})₂, halo-substituted (C₁-C₄ alkoxy), unsubstituted C₁-C₆ alkylthio, hydroxy-substituted (C₂-C₄ alkoxy), (unsubstituted C₂-C₄ alkoxy)-O—P(O)(OH)₂, (unsubstituted C₂-C₄ alkoxy)-O—P(O)(OR^{10A})₂, unsubstituted C₁-C₄ alkoxy(unsubstituted C₁-C₄ alkoxy); R^{10A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy, or unsubstituted alkoxy.
R^{10B} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R¹¹-substituted or unsubstituted phenyl, R¹-substituted or unsubstituted heteroaryl, R¹¹-substituted or unsubstituted heterocycloalkyl, or R¹-substituted or unsubstituted heteroaryl; wherein said R¹¹-substituted or unsubstituted phenyl, R¹-substituted or unsubstituted heteroaryl, R¹¹-substituted or unsubstituted heterocycloalkyl, and R¹¹-substituted or unsubstituted heteroaryl are optionally substituted with one or more R¹¹ substituents;
R¹¹ is independently halogen, CN, CF₃, unsubstituted C₁-C₄ saturated alkyl, halo-substituted (saturated C₁-C₄ alkyl), unsubstituted C₂-C₄ alkenyl, unsubstituted C₂-C₄ alkynyl, or unsubstituted C₃-C₆ cycloalkyl.
R² is independently R²⁰-substituted or unsubstituted C₁-C₂₀ saturated alkyl, R²⁰-substituted or unsubstituted C₂-C₂₀ alkenyl, R²⁰-substituted or unsubstituted C₂-C₂₀ alkynyl, R²⁰-substituted or unsubstituted C₃-C₁₀ cycloalkyl, R²⁰-substituted or unsubstituted phenyl, R¹¹-substituted or unsubstituted 6-10 membered aryl, R²⁰-substituted or unsubstituted 4-10 membered heterocycloalkyl, or R²⁰-substituted or unsubstituted 5-10 membered heteroaryl;
wherein said R²⁰-substituted C₁-C₂₀ saturated alkyl, R²⁰-substituted C₂-C₂₀ alkenyl, R²⁰-substituted C₂-C₂₀ alkynyl, R²⁰-substituted C₃-C₁₀ cycloalkyl, R²⁰-substituted phenyl, R²⁰-substituted 6-10 membered aryl, R²⁰-substituted 4-10 membered heterocycloalkyl, and R²⁰-substituted 5-10 membered heteroaryl is substituted with 1 to 4 R²⁰ substituents;
each R²⁰ substituent is independently halogen, —CN, —CF₃, —OR^{20B}, —OC(O)R^{20B}, —OC(O)NR^{20A}R^{20B}, —C(O)—OR^{20B}, —C(O)NR^{20A}R^{20B}, —SOR^{20B}, —SO₂R^{20B}, —SO₂NR^{20A}R^{20B}, —O—P(O)(OR^{20A})₂, —N^{20A}R^{20B}, —NR^{20A}C(O)R^{20B}, —NR^{20A}SOR^{20B}, —NR^{20A}C(O)OR^{20B}, —N^{20A}SO₂R^{20B}, —C(O)R^{20B}, unsubstituted C₁-C₆ saturated alkyl, unsubstituted C₂-C₆ alkenyl, unsubstituted C₂-C₆ alkynyl, halo-substituted (C₁-C₄ alkyl), hydroxy-substituted (C₁-C₄ alkyl), (unsubstituted C₁-C₄ alkyl)-O—P(O)(OH)₂, (unsubstituted C₁-C₄ alkyl)-O—P(O)OR^{20A})₂, halo-substituted (C₁-C₄ alkoxy), unsubstituted C₁-C₆ alkylthio, hydroxy-substituted (C₂-C₄ alkoxy), (unsubstituted C₂-C₄ alkoxy)-O—P(O)(OH)₂, (unsubstituted C₂-C₄ alkoxy)-O—P(O)(OR^{20A})₂, unsubstituted C₁-C₄ alkoxy(unsubstituted C₁-C₄ alkoxy);
R^{20A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl, or unsubstituted alkoxy-substituted alkyl or wherein R′ and R″, together with the nitrogen atom to which they are attached form a heterocycloalkyl or heteroaryl optionally substituted with one or more halo, oxo, hydroxy, or unsubstituted alkoxy
R^{20B} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R²¹-substituted or unsubstituted phenyl, R²¹-substituted or unsubstituted heteroaryl, R²¹-substituted or unsubstituted heterocycloalkyl, or R²¹-substituted or unsubstituted heteroaryl; wherein said R²¹-substituted or unsubstituted phenyl, R²¹-substituted or unsubstituted heteroaryl, R²¹-substituted or unsubstituted heterocycloalkyl, and R²¹-substituted or unsubstituted heteroaryl are optionally substituted with one or more R²¹ substituents;
R²¹ is independently halogen, CN, CF₃, unsubstituted C₁-C₄ saturated alkyl, halo-substituted (saturated C₁-C₄ alkyl), unsubstituted C₂-C₄ alkenyl, unsubstituted C₂-C₄ alkynyl, or unsubstituted C₃-C₆ cycloalkyl.
R³ is independently halogen, —CN, —CF₃, —C(O)NH₂, —C(O)OR³, —O—P(O)(OH)₂, —NR^{3A}R^{3B}, —C(O)R^{3C}, —NR^{3A}C(O)R^{3C}, —OR^{3D}, —NR^{3A}SO₂R^{3D}, unsubstituted C₂-C₆ saturated alkyl-NR^{3E}— unsubstituted C₂-C₆ saturated alkyl, N(R^{3H})SO₂(unsubstituted C₁-C₆ saturated alkyl)-N(R^{3H}(R³), N(R^{3H})CO(unsubstituted C₁-C₆ saturated alkyl)-N(R^{3H}(R^{3I}), unsubstituted C₁-C₆ saturated alkyl, unsubstituted C₂-C₆ alkenyl, unsubstituted C₂-C₆ alkynyl, unsubstituted C₁-C₆ saturated alkylthio, or unsubstituted C₁-C₆ cycloalkyl;
R^{3A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy, or unsubstituted alkoxy;
R^{3B}, R^{3C}, and R^{3D} are independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R³¹-substituted or unsubstituted phenyl, R³¹-substituted or unsubstituted heteroaryl, R³¹-substituted or unsubstituted heterocycloalkyl, or R³-substituted or unsubstituted heteroaryl; wherein said R³¹-substituted or unsubstituted phenyl, R³¹-substituted or unsubstituted heteroaryl, R³¹-substituted or unsubstituted heterocycloalkyl, and R³¹-substituted or unsubstituted heteroaryl are optionally substituted with one or more R³ substituents;
R³¹ is independently halogen, CN, CF₃, unsubstituted C₁-C₄ saturated alkyl, halo-substituted (saturated C₁-C₄ alkyl), unsubstituted C₂-C₄ alkenyl, unsubstituted C₂-C₄ alkynyl, or unsubstituted C₃-C₆ cycloalkyl.
R^{3J} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, substituted or unsubstituted aryl, substituted or unsubstituted heteroaryl, or substituted or unsubstituted heterocycloalkyl, wherein said substituted aryl, substituted heteroaryl, and substituted heterocyclic groups are independently substituted with one or more halogen, CN, CF₃, or unsubstituted C₁-C₄ saturated alkyl;
R^{3E} is independently hydrogen, —C(O)R^{3F}, —C(O)OR^{3F}, —SOR^{3F}, —SO₂R^{3F}, —C(O)NR^{3F}R^{3G}, or —SO₂NR^{3F}R^{3G};
R^{3G} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy;
R^{3F} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R³¹-substituted or unsubstituted phenyl, R³¹-substituted or unsubstituted heteroaryl, R³¹-substituted or unsubstituted heterocycloalkyl, or R³¹-substituted or unsubstituted heteroaryl; wherein said R³¹-substituted or unsubstituted phenyl, R³¹-substituted or unsubstituted heteroaryl, R³¹-substituted or unsubstituted heterocycloalkyl, and R³¹-substituted or unsubstituted heteroaryl are optionally substituted with one or more R³¹ substituents;
R^{3H} and R^{3I} are independently hydrogen, substituted or unsubstituted C₁-C₆ saturated alkyl, wherein said substituted saturated alkyl is independently substituted with one or more halogen, OH, amine, CN, CF₃ or unsubstituted C₁-C₄ saturated alkyl; or R^{3H} and R^{3I}, taken together with the atom or atoms through which they are connected to form a substituted or unsubstituted 5-6 membered heterocycloalkyl or substituted or unsubstituted 5-6 membered heteroaryl, wherein the substituted 5-6 membered heterocycloalkyl or substituted 5-6 membered heteroaryl is independently substituted with one or more halogen, OH, amine, CN, CF₃, or unsubstituted C₁-C₄ saturated alkyl, wherein said amine substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy, or unsubstituted alkoxy;
R⁴ is independently halogen, —CN, —CF₃, —C(O)NH₂, —C(O)OR^{4J}, —O—P(O)(OH)₂, —NR^{4A}R^{4B}, —C(O)R^{4C}, —NR^{4A}C(O)R^{4C}, —OR, —NR^{4A}SO₂R^{4D}, unsubstituted C₂-C₆ saturated alkyl-NR^{4E}— unsubstituted C₂-C₆ saturated alkyl, N(R^{4H})SO₂(unsubstituted C₁-C₆ saturated alkyl)-N(R^{4H}(R^{4I}), N(R^{4H})CO(unsubstituted C₁-C₆ saturated alkyl)-N(R^{4H}) R^{4I}), unsubstituted C₁-C₆ saturated alkyl, unsubstituted C₂-C₆ alkenyl, unsubstituted C₂-C₆ alkynyl, unsubstituted C₁-C₆ saturated alkylthio, or unsubstituted C₁-C₆ cycloalkyl;
R^{4A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl, or unsubstituted alkoxy-substituted alkyl or wherein R′ and R″, together with the nitrogen atom to which they are attached form a heterocycloalkyl or heteroaryl optionally substituted with one or more halo, oxo, hydroxy, or unsubstituted alkoxy;
R^{4B}, R^{4C}, and R^{4D} are independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R⁴¹-substituted or unsubstituted phenyl, R⁴¹-substituted or unsubstituted heteroaryl, R⁴¹-substituted or unsubstituted heterocycloalkyl, or R⁴-substituted or unsubstituted heteroaryl; wherein said R⁴¹-substituted or unsubstituted phenyl, R⁴¹-substituted or unsubstituted heteroaryl, R⁴¹-substituted or unsubstituted heterocycloalkyl, and R⁴¹-substituted or unsubstituted heteroaryl are optionally substituted with one or more R⁴ substituents;
R⁴¹ is independently halogen, CN, CF₃, unsubstituted C₁-C₄ saturated alkyl, halo-substituted (saturated C₁-C₄ alkyl), unsubstituted C₂-C₄ alkenyl, unsubstituted C₂-C₄ alkynyl, or unsubstituted C₃-C₆ cycloalkyl.
R^{4J} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, substituted or unsubstituted aryl, substituted or unsubstituted heteroaryl, or substituted or unsubstituted heterocycloalkyl, wherein said substituted aryl, substituted heteroaryl, and substituted heterocyclic groups are independently substituted with one or more halogen, CN, CF₃, or unsubstituted C₁-C₄ saturated alkyl;
R^{4E} is independently hydrogen, —C(O)R^{4F}, —C(O)OR^{4F}, —SOR^{4F}, —SO₂R^{4F}, —C(O)NR^{4F}R^{4G}, or —SO₂NR^{4F}R^{4G};
R^{4G} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy, or unsubstituted alkoxy;
R^{4F} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R⁴¹-substituted or unsubstituted phenyl, R⁴¹-substituted or unsubstituted heteroaryl, R⁴¹-substituted or unsubstituted heterocycloalkyl, or R⁴-substituted or unsubstituted heteroaryl; wherein said R⁴¹-substituted or unsubstituted phenyl, R⁴¹-substituted or unsubstituted heteroaryl, R⁴¹-substituted or unsubstituted heterocycloalkyl, and R⁴¹-substituted or unsubstituted heteroaryl are optionally substituted with one or more R⁴¹ substituents;
R^{4H} and R^{4I} are independently hydrogen, substituted or unsubstituted C₁-C₆ saturated alkyl, wherein said substituted saturated alkyl is independently substituted with one or more halogen, OH, amine, CN, CF₃, or unsubstituted C₁-C₄ saturated alkyl; or R^{4H} and R^{4I}, taken together with the atom or atoms through which they are connected to form a substituted or unsubstituted 5-6 membered heterocycloalkyl or substituted or unsubstituted 5-6 membered heteroaryl, wherein the substituted 5-6 membered heterocycloalkyl or substituted 5-6 membered heteroaryl is independently substituted with one or more halogen, OH, amine, CN, CF₃, or unsubstituted C₁-C₄ saturated alkyl, wherein said amine substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy;
L is halo-substituted (C₁-C₁₀ saturated alkylene), R^{L}-substituted or unsubstituted C₁-C₁₀ alkylene, R^{L}-substituted or unsubstituted C₂-C₁₀ alkenylene, R^{L}-substituted or unsubstituted C₂-C₁₀ alkynylene, R^{L}-substituted or unsubstituted C₂-C₆ saturated alkylene-O—C₂-C₆ saturated alkylene, R^{L}-substituted or unsubstituted C₂ saturated alkylene-O—C₂ saturated alkylene, R^{L}-substituted or unsubstituted C₂ saturated alkylene-O—C₂ saturated alkylene-O—C₂ saturated alkylene, R^{L}-substituted or unsubstituted C₂-C₆ saturated alkylene-NR^{L}—C₂-C₆ saturated alkylene, R^{L}-substituted or unsubstituted C₃-C₆ cycloalkylene, R^{L}-substituted or unsubstituted phenylene, R^{L}-substituted or unsubstituted 4-6 membered heterocycloalkylene, R^{L}-substituted or unsubstituted 5-6 membered heteroarylene, R^{L}-substituted or unsubstituted C₁-C₄ saturated alkylene-(C₃-C₆ cycloalkylene)-C₁-C₄ saturated alkylene, R^{L}-substituted or unsubstituted C₁-C₄ saturated alkylene-phenylene-C₁-C₄ saturated alkylene, R^{L}-substituted or unsubstituted C₁-C₄ saturated alkylene-(4-6 membered heterocycloalkylene)-C₁-C₄ saturated alkylene, or R^{L}-substituted or unsubstituted C₁-C₄ saturated alkylene-(5-6 membered heteroarylene)-C₁-C₄ saturated alkylene;
wherein said R^{L}-substituted C₁-C₁₀ alkyl, R^{L}-substituted C₂-C₁₀ alkenyl, R^{L}-substituted C₂-C₁₀ alkynyl, R^{L}-substituted C₂-C₆ saturated alkyl-O—C₂-C₆ saturated alkyl, R^{L}-substituted C₂ saturated alkyl-O—C₂ saturated alkyl, R^{L}-substituted C₂ saturated alkyl-O—C₂ saturated alkyl-O—C₂ saturated alkyl, R^{L}-substituted C₂-C₆ saturated alkyl-NR^{L4}—C₂-C₆ saturated alkyl, R^{L}-substituted C₃-C₆ cycloalkyl, R^{L}-substituted phenyl, R^{L}-substituted 4-6 membered heterocycloalkyl, R^{L}-substituted 5-6 membered heteroaryl, R^{L}-substituted C₁-C₄ saturated alkyl-(C₃-C₆ cycloalkyl)-C₁-C₄ saturated alkyl, R^{L}-substituted C₁-C₄ saturated alkyl-phenyl-C₁-C₄ saturated alkyl, R^{L}-substituted C₁-C₄ saturated alkyl-(4-6 membered heterocycloalkyl)-C₁-C₄ saturated alkyl, and R^{L}-substituted C₁-C₄ saturated alkyl-(5-6 membered heteroaryl)-C₁-C₄ saturated alkyl are independently substituted with 1 or 2 R^{L}; R^{L} is independently halogen, —OR^{LB}, —O—P(O)(OR^{LA})₂, —NR^{LA}R^{LB}, —OC(O)R^{LB}, —C(O)—OR^{LB}, —SOR^{LB}, —SO₂R^{LB}, —C(O)NR^{LA}R^{LB}, —SO₂NR^{L A}R^{LB}, —OC(O)NR^{LA}R^{LB}, —NR^{LA}C(O)R^{LB}, —NR^{LA}SOR^{LB}, —NR^{LA}C(O)OR^{LB}, —NR^{LA}SO₂R^{LB}, —R^{LA}S O₂R^{LB}, halo-substituted (saturated C₁-C₄alkyl), (unsubstituted C₁-C₄ saturated alkyl)(unsubstituted C₁-C₄ saturated alkyl)amino, unsubstituted C₁-C₄ saturated alkyl, hydroxy-substituted (C₁-C₄ saturated alkyl), (unsubstituted C₁-C₄ saturated alkyl)-O—P(O)(OH)₂, (unsubstituted C₁-C₄ saturated alkyl)-O—P(O)OR^{LA})₂, halo-substituted (C₁-C₄ alkoxy), unsubstituted C₁-C₄ alkoxy, unsubstituted C₁-C₆ saturated alkylthio, hydroxy-substituted (C₂-C₄ alkoxy), (unsubstituted C₂-C₄ alkoxy)-O—P(O)(OH)₂, (unsubstituted C₂-C₄alkoxy)-O—P(O)(OR^{LA})₂, unsubstituted C₁-C₄ alkoxy(unsubstituted C₁-C₄ alkoxy), COR^{LA}, or CON(R^{LA})(R^{LC});
R^{LA} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy;
R^{LB} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R^{L6}-substituted or unsubstituted phenyl, R^{L6}-substituted or unsubstituted heteroaryl, R^{L6}-substituted or unsubstituted heterocycloalkyl, or R^{L6}-substituted or unsubstituted heteroaryl; wherein said R^{L6}-substituted or unsubstituted phenyl, R^{L6}-substituted or unsubstituted heteroaryl, R^{L6}-substituted or unsubstituted heterocycloalkyl, and R^{L6}-substituted or unsubstituted heteroaryl are optionally substituted with one or more R^{L6} substituents;
R^{L6} is independently halogen, CN, CF₃, unsubstituted C₁-C₄ saturated alkyl, halo-substituted (saturated C₁-C₄ alkyl), unsubstituted C₂-C₄ alkenyl, unsubstituted C₂-C₄ alkynyl, or unsubstituted C₃-C₆ cycloalkyl.
R^{LC} is independently hydrogen, substituted or unsubstituted C₁-C₆ saturated alkyl, wherein said substituted saturated alkyl is independently substituted with one or more halogen, OH, amine, CN, CF₃, or unsubstituted C₁-C₄ saturated alkyl;
R^{L4} is independently hydrogen, COR^{L4A}, CO₂H, CO₂R^{L4A}, SOR^{L4A}, SO₂R^{L4A}, CONH₂, CONR^{L4A}R^{L4B}, SO₂NH₂, or SO₂NR^{L4A}R^{L4B};
R^{L4B} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy;
R^{L4A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R^{L6}-substituted or unsubstituted phenyl, R^{L6}-substituted or unsubstituted heterocycloalkyl, or R^{L6}-substituted or unsubstituted heteroaryl; wherein said R^{L6}-substituted or unsubstituted phenyl, R^{L6}-substituted or unsubstituted heterocycloalkyl, and R^{L6}-substituted or unsubstituted heteroaryl are optionally substituted with one or more R^{L6} substituents;
L¹ is independently ═NC(O)—, —C(O)N(R^{L1})—, or —N(R^{L1})C(O)—;
R^{L1} is independently hydrogen, unsubstituted C₁-C₆ saturated alkyl, unsubstituted C₂-C₆ alkenyl, unsubstituted C₂-C₆ alkynyl, or unsubstituted C₃-C₆ cycloalkyl;
L² is independently=NC(O)—, —C(O)N(R^{L2})—, or —N(RU)C(O)—;
R^{L2} is independently hydrogen, unsubstituted C₁-C₆ saturated alkyl, unsubstituted C₂-C₆ alkenyl, unsubstituted C₂-C₆ alkynyl, or unsubstituted C₃-C₆ cycloalkyl;
A⁵ is independently O, S, N, NR⁵, or CR⁵;
R⁵ is independently hydrogen, unsubstituted C₁-C₆ saturated alkyl, unsubstituted C₂-C₆ alkenyl, unsubstituted C₂-C₆ alkynyl, or unsubstituted C₃-C₆ cycloalkyl;
A⁶ is independently O, S, N, NR, or CR⁶;
R⁶ is independently hydrogen, unsubstituted C₁-C₆ saturated alkyl, unsubstituted C₂-C₆ alkenyl, unsubstituted C₂-C₆ alkynyl, or unsubstituted C₃-C₆ cycloalkyl;
A⁷ is independently N or CR⁷;
R⁷ is independently hydrogen, unsubstituted C₁-C₆ saturated alkyl, unsubstituted C₂-C₆ alkenyl, unsubstituted C₂-C₆ alkynyl, or unsubstituted C₃-C₆ cycloalkyl;
A⁸ is independently N or CR⁸;
R⁸ is independently hydrogen, unsubstituted C₁-C₆ saturated alkyl, unsubstituted C₂-C₆ alkenyl, unsubstituted C₂-C₆ alkynyl, or unsubstituted C₃-C₆ cycloalkyl;
A⁹ is independently N or CR⁹;
R⁹ is independently hydrogen, halogen, —CN, —CF₃, O—P(O)(OR^{9A})₂, —OR^{9D}, —NR^{9B}R^{9B}, —NR^{9A}R^{9B}, —OC(O)NR^{9A}R^{9B}, —OC(O)R^{9C}, —C(O)R^{9C}, —C(O)—OR^{9J}, —C(O)NR^{9A}R^{9B}, —SOR^{9D}, —SO₂NR^{9A}R^{9B}, —SO₂R^{9J}, —NR^{9A}SO₂R^{9D}, —NR^{9A}C(O)R^{9C}, N(R^{9H})SO₂(unsubstituted C₁-C₄ saturated alkyl)-N(R^{9I})(R^{9H}), N(R^{9H})CO(unsubstituted C₁-C₄ saturated alkyl)-N(R^{9I}(R^{9H}), O(unsubstituted C₁-C₄ saturated alkyl)-N(R^{9I})(R^{9H}), O(unsubstituted C₁-C₄ saturated alkyl)-OR^{9B}, NR^{9H}(unsubstituted C₁-C₄ saturated alkyl)-N(R^{9I})(R^{9H}), halo-substituted (C₁-C₁₀ saturated alkyl), halo-substituted (C₁-C₁₀ alkoxy), R⁹⁰-substituted or unsubstituted (C₁-C₁₀ saturated alkyl)oxy, R⁹⁰-substituted or unsubstituted (C₁-C₁₀ saturated alkyl)amino, R⁹⁰-substituted or unsubstituted (C₁-C₆ saturated alkyl)(C₁-C₄ saturated alkyl)amino, R⁹⁰-substituted or unsubstituted phenyl, R⁹⁰-substituted or unsubstituted 5-6 membered heterocycloalkyl, or R⁹⁰-substituted or unsubstituted 5-6 membered heteroaryl;
R^{9A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy
R^{9B}, R^{9C}, and R^{9D} are independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R⁹¹-substituted or unsubstituted phenyl, R⁹¹-substituted or unsubstituted heteroaryl, R⁹¹-substituted or unsubstituted heterocycloalkyl, or R⁹¹-substituted or unsubstituted heteroaryl; wherein said R⁹¹-substituted or unsubstituted phenyl, R⁹¹-substituted or unsubstituted heteroaryl, R⁹¹-substituted or unsubstituted heterocycloalkyl, and R⁹¹-substituted or unsubstituted heteroaryl are optionally substituted with one or more R⁹¹ substituents;
R⁹¹ is independently halogen, CN, CF₃, unsubstituted C₁-C₄ saturated alkyl, halo-substituted (saturated C₁-C₄ alkyl), unsubstituted C₂-C₄ alkenyl, unsubstituted C₂-C₄ alkynyl, or unsubstituted C₃-C₆ cycloalkyl.
R⁹¹ is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, substituted or unsubstituted aryl, substituted or unsubstituted heteroaryl, or substituted or unsubstituted heterocycloalkyl, wherein said substituted aryl, substituted heteroaryl, and substituted heterocyclic groups are independently substituted with one or more halogen, CN, CF₃, or unsubstituted C₁-C₄ saturated alkyl;
R^{9H} and R^{9I} are independently hydrogen, substituted or unsubstituted C₁-C₆ saturated alkyl, wherein said substituted saturated alkyl is independently substituted with one or more halogen, OH, amine, CN, CF₃, or unsubstituted C₁-C₄ saturated alkyl; or R^{9H} and R^{9I}, taken together with the atom or atoms through which they are connected to form a substituted or unsubstituted 5-6 membered heterocycloalkyl or substituted or unsubstituted 5-6 membered heteroaryl, wherein the substituted 5-6 membered heterocycloalkyl or substituted 5-6 membered heteroaryl is independently substituted with one or more halogen, OH, amine, CN, CF₃, or unsubstituted C₁-C₄ saturated alkyl, wherein said amine substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy;
wherein said R⁹⁰-substituted (C₁-C₁₀ saturated alkyl)oxy, R⁹⁰-substituted (C₁-C₁₀ saturated alkyl)amino, R⁹⁰-substituted (C₁-C₆ saturated alkyl)(C₁-C₄ saturated alkyl)amino, R⁹⁰-substituted phenyl, R⁹⁰-substituted 5-6 membered heterocycloalkyl, or R⁹⁰-substituted 5-6 membered heteroaryl is substituted with 1-4 independent R′ and
R⁹⁰ is halogen, —OR^{90B}, —O—P(O)(OR^{90A})₂, —NR^{90A}R^{90B}, —NR^{90B}R^{90B}, —C(O)—OR^{90B}, —C(O)NR^{90A}R^{90B}, —SOR^{90B}, —SO₂R^{90B}, —SO₂NR^{90A}R^{90B}, —OC(O)R^{90B}, —OC(O)NR^{90A}R^{90B}, —NR^{90A}C(O)R^{90B}, —NR^{90A}SOR^{90B}, —NR^{90A}C(O)OR^{90B}, —C(O)R^{90B}, —SO₂R^{90J}, —C(O)—OR^{90J}, —C(O)NR^{90A}R^{90H}, —C(O)R^{90A}, (unsubstituted C₁-C₄ saturated alkyl)(unsubstituted C₁-C₄ saturated alkyl)amino, unsubstituted C₁-C₄ saturated alkyl, halo-substituted (C₁-C₄ saturated alkyl), hydroxy-substituted (C₁-C₄ saturated alkyl), (unsubstituted C₁-C₄ saturated alkyl)-O—P(O)(OH)₂, (unsubstituted C₁-C₄ saturated alkyl)-O—P(O)(OR^{90A})₂, halo-substituted (C₁-C₄ alkoxy), unsubstituted C₁-C₄ alkoxy, hydroxy-substituted (C₂-C₄ alkoxy), (unsubstituted C₂-C₄ alkoxy)-O—P(O)(OH)₂, (unsubstituted C₂-C₄alkoxy)-O—P(O)(OR^{90A})₂, or unsubstituted C₁-C₄ alkoxy-(unsubstituted C₁-C₄ alkoxy);
R^{90A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy;
R^{90B} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R⁹¹-substituted or unsubstituted phenyl, R⁹¹-substituted or unsubstituted heteroaryl, R⁹¹-substituted or unsubstituted heterocycloalkyl, or R⁹¹-substituted or unsubstituted heteroaryl; wherein said R⁹¹-substituted or unsubstituted phenyl, R⁹¹-substituted or unsubstituted heteroaryl, R⁹¹-substituted or unsubstituted heterocycloalkyl, and R⁹¹-substituted or unsubstituted heteroaryl are optionally substituted with one or more R⁹¹ substituents;
R⁹¹ is independently halogen, CN, CF₃, unsubstituted C₁-C₄ saturated alkyl, halo-substituted (saturated C₁-C₄ alkyl), unsubstituted C₂-C₄ alkenyl, unsubstituted C₂-C₄ alkynyl, or unsubstituted C₃-C₆ cycloalkyl.
R^{90J} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, substituted or unsubstituted aryl, substituted or unsubstituted heteroaryl, or substituted or unsubstituted heterocycloalkyl, wherein said substituted aryl, substituted heteroaryl, and substituted heterocyclic groups are independently substituted with one or more halogen, CN, CF₃, or unsubstituted C₁-C₄ saturated alkyl;
R^{90H} is independently hydrogen, substituted or unsubstituted C₁-C₆ saturated alkyl, wherein said substituted saturated alkyl is independently substituted with one or more halogen, OH, amine, CN, CF₃, or unsubstituted C₁-C₄ saturated alkyl, wherein said amine substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy; or a tautomer thereof; or a salt thereof (e.g., a pharmaceutically acceptable salt).

### BRIEF DESCRIPTION OF THE DRAWINGS

FIGS. 1A-1B. Western blot analysis of total and phosphorylated protein of STING, TBK1, and IRF3 in THP-1 cells. Cells were treated by 10 uM compounds for 3 h. Representative results of two independent experiments were shown. FIG. 1A. Western blot analysis of total and phosphorylated protein of STING, TBK1, and IRF3 in THP-1 cells. Cells were treated by 10 μM compounds for 3 h. Representative results of two independent experiments were shown. FIG. 1B. Western blot analysis of total and phosphorylated protein of STING, TBK1, and IRF3 in THP-1 cells. Cells were treated by 10 μM compounds for 3 h. Representative results of two independent experiments were shown.

FIGS. 2A-2B. qPCR analysis of mRNA levels of IFNB, ISG15, and CXCL10 in THP1 cells with the compounds. Cells were treated by 10 uM compounds for 6 h. Representative results of two independent experiments were shown. Statistical analysis was carried out using one-way anova. Ns, not significant, ** p<0.01. FIG. 2A. Effects of Cpd2 (Example 2), Cpd3 (Example 3), Cpd4 (Example 4) and Cpd9 (Example 9) on STING activation. qPCR analysis of mRNA levels of IFNB, ISG15 and CXCL10 in THP-1 cells. Cells were treated by 10 μM chemicals for 6 h. Representative results of two independent experiments were shown. Statistical analysis was carried out using one-way anova. Ns, not significant, * p<0.05. FIG. 2B. Effects of Cpd4 (Example 4), Cpd5 (Example 4), Cpd6 (Example 6), Cpd7 (Example 7), Cpd8 (Example 8), Cpd10 (Example 10) and Cpd11 (Example 11) on STING activation. aPCR analysis of mRNA levels of IFNB, ISG15 and CXCL10 in THP1 cells. Cells were treated by 10 μM chemicals for 6 h. Representative results of two independent experiments were shown. Statistical analysis was carried out using one-way anova. Ns, not significant, ** p<0.01.

FIG. 3. Effects of Cpd 4 (Example 4) on tumor volume in the s.c. CT-26 mouse colon adenocarcinoma model in BALB/c mice. Statistical analysis was carried out using one-way Anova, p<0.001.

FIG. 4. Effects of Cpd 4 (Example 4) on mouse body weight in CT-26 mouse colon adenocarcinoma model in BALB/c mice. Statistical analysis was carried out using one-way Anova, p<0.001.

FIG. 5. T/C value of Cpd 4 (Example 4) on tumor growth in CT-26 mouse colon adenocarcinoma model in BALB/c mice. Statistical analysis was carried out using one-way Anova, p<0.001.

### I. Definitions

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O is equivalent to —OCH₂—.

The terms “halo” or “halogen” refers to fluorine, chlorine, bromine or iodine. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

In embodiments, the term “haloalkyl” refers to an alkyl group, in certain embodiments, C₁₋₆alkyl group in which one or more of the hydrogen atoms are replaced by halogen. In embodiments, such groups include, but are not limited to, chloromethyl, trifluoromethyl, 1-chloro-2-fluoroethyl, 2,2-difluoroethyl, 2-fluoropropyl, 2-fluoropropan-2-yl, 2,2,2-trifluoroethyl, 1, 1-difluoroethyl, 1,3-difluoro-2-methylpropyl, 2,2-difluorocyclopropyl, (trifluoromethyl)cyclopropyl, 4,4-difluorocyclohexyl and 2,2,2-trifluoro-1, 1-dimethyl-ethyl.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-, and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs, and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

In embodiments, the term “alkyl” refers to a straight or branched hydrocarbon chain group consisting solely of carbon and hydrogen atoms, containing no unsaturation (i.e., saturated alkyl); having from one to ten, one to eight, one to six, or one to four carbon atoms; and which is attached to the rest of the molecule by a single bond, e.g., methyl, ethyl, n-propyl, 1-methylethyl (iso-propyl), n-butyl, n-pentyl, 1, 1-dimethylethyl (t-butyl), and the like.

The term “alkenyl” refers to a straight or branched hydrocarbon chain group consisting solely of carbon and hydrogen atoms (unless substituted in which case one or more hydrogen atoms is replaced with a substitution), containing at least one double bond, in certain embodiment, having from 2 to 10 carbon atoms, from 2 to 8 carbon atoms, or from 2 to 6 carbon atoms, and which is attached to the rest of the molecule by a single bond or a double bond, e.g., ethenyl, prop-1-enyl, but-1-enyl, pent-1-enyl, penta-1,4-dienyl, and the like.

The term “alkynyl” refers to a straight or branched hydrocarbon chain group consisting solely of carbon and hydrogen atoms (unless substituted in which case one or more hydrogen atoms is replaced with a substitution), containing at least one triple bond, having from two to ten carbon atoms, and which is attached to the rest of the molecule by a single bond or a triple bond, e.g., ethynyl, prop-1-ynyl, but-1-ynyl, pent-1-ynyl, pent-3-ynyl, and the like.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

In embodiments, the term “alkylene” and “alkylene chain” refer to a straight or branched divalent hydrocarbon chain consisting solely of carbon and hydrogen, containing no unsaturation and having from one to eight carbon atoms, e.g., methylene, ethylene, propylene, n-butylene, and the like. In embodiments, the alkylene chain may be attached to the rest of the molecule through any two carbons within the chain.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in addition to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′-represents both —C(O)₂R′- and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. A heteroalkenylene inlcudes one or more double bonds. A heteroalkynylene includes one or more triple bonds.

In embodiments, the term “alkoxy” refers to the group having the formula —OR wherein R is alkyl or haloalkyl, where the alkyl may be optionally substituted by one or more substituents, in one embodiment, one, two or three substitutents independently selected from the group consisting of nitro, halo, hydroxyl, alkoxy, oxo, thioxo, amino, carbony, carboxy, azido, cyano, cycloalkyl, heteroaryl, and heterocyclyl.

The term “alkoxyalkyl” refers to a group having the formula —RₕOR wherein Rₕ is a straight or branched alkylene chain and OR is alkoxy as defined above.

The term “alkylthio” refers to a group having the formula —SR wherein R is alkyl or haloalkyl.

The term “aryloxy” refers to the group —OR, in which R is aryl, including lower aryl, such as phenyl.

The term “amine” or “amino” refers to a group having the formula —NR′R″ wherein R′ and R″ are each independently hydrogen, alkyl, haloalkyl, hydroxyalkyl, or alkoxyalkyl or wherein R′ and R″, together with the nitrogen atom to which they are attached form a heterocyclyl optionally substituted with halo, oxo, hydroxy, or alkoxy.

The term “aminoalkyl” refers to a group having the formula —RₕNR′R″ wherein R is a straight or branched alkylene chain and wherein NR′R″ is amino as defined above.

The term “aminocarbonyl” refers to a group having the formula —C(O)NR′R″ wherein —NR′R″ is amino as defined above.

The terms “cycloalkyl” and “heterocycloalkyl,” by themselves or in combination with other terms, mean, unless otherwise stated, cyclic versions of “alkyl” and “heteroalkyl,” respectively. Cycloalkyl is not aromatic. In embodiment heterocycloalkyl is not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term ‘cycloalkyl” herein alone or as part of another group refers to fully saturated and partially unsaturated hydrocarbon rings of 3 to 9 carbon atoms. In embodiments, examples include cyclopropyl, cyclobutyl, cyclopentyl and cyclohexyl, and the like. In embodiments, a cycloalkyl may be substituted. In embodiments, a substituted cycloalkyl refers to such rings having one, two, or three substituents, selected from the group consisting of halo, alkyl, substituted alkyl, alkenyl, alkynyl, nitro, cyano, oxo (═O), hydroxy, alkoxy, thioalkyl, —CO₂H, —C(═O)H, CO₂-alkyl, —C(═O)alkyl, keto, ═N—OH, ═N—O-alkyl, aryl, heteroaryl, heterocyclo, —NR′R″, —C(═O)NR′R″, —CO₂NR′R″, —C(═O)NR′R″, —NR′CO₂R″, —NR′C(═O)R″, —SO₂NR′R″, and —NR′SO₂R″, wherein each of R′ and R″ are independently selected from hydrogen, alkyl, substituted alkyl, and cycloalkyl, or R′ and R″ together form a heterocyclo or heteroaryl ring.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. In embodiments, a bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. A bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings.

The term “cycloalkylalkyl” refers to a group of the formula —RₐR_{d} where Rₐ is an alkyl group as defined above and R_{d} is a cycloalkyl group as defined above. The alkyl group and the cycloalkyl group may be optionally substituted as defined herein.

In embodiments, the term “heterocycloalkyl” means a monocyclic, bicyclic, or a multicyclic heterocycloalkyl ring system. In embodiments, heterocycloalkyl groups are fully saturated. In embodiments, a bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

In embodiments, a heterocycloalkyl is a “heterocyclyl”, which refers to a stable 3- to 15-membered ring group which consists of carbon atoms and from one to five heteroatoms selected from a group consisting of nitrogen, oxygen and sulfur. In one embodiment, the heterocyclic ring system group may be a monocyclic, bicyclic or tricyclic ring or tetracyclic ring system, which may include fused or bridged ring systems; and the nitrogen or sulfur atoms in the heterocyclic ring system group may be optionally oxidized; the nitrogen atom may be optionally quaternized; and the heterocyclyl group may be partially or fully saturated or aromatic. The heterocyclic ring system may be attached to the main structure at any heteroatom or carbon atom which results in the creation of a stable compound. Exemplary heterocylic radicals include, azetidinyl, benzopyranonyl, benzopyranyl, benzotetrahydrofuranyl, benzotetrahydrothienyl, chromanyl, chromonyl, coumarinyl, decahydroisoquinolinyl, dibenzofuranyl, dihydrobenzisothiazinyl, dihydrobenzisoxazinyl, dihydrofuryl, dihydropyranyl, dioxolanyl, dihydropyrazinyl, dihydropyridinyl, dihydropyrazolyl, dihydropyrimidinyl, dihydropyrrolyl, dioxolanyl, 1,4 dithianyl, isobenzotetrahydrofuranyl, isobenzotetrahydrothienyl, isochromanyl, isocoumarinyl, benzo[1,3]dioxol-5-yl, benzodioxolyl, 1,3-dioxolan-2-yl, dioxolanyl, morpholinyl, octahydroindolyl, octahydroisoindolyl, tetrahydrofuran, oxazolidin-2-onyl, oxazolidinonyl, piperidinyl, piperazinyl, pyranyl, tetrahydroiuryl, tetrahydrofuranyl, tetrahydroisoquinolinyl, tetrahydropyranyl, tetrahydrothienyl, pyrrolidinonyl, oxathiolanyl, and pyrrolidinyl.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. In embodiments, a fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. In embodiments, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substitutents described herein.

In embodiments, the term “aryl” refers to a group of carbocylic ring systems, including monocyclic, bicyclic, tricyclic, tetracyclic C₆-C₁₈ ring systems, wherein at least one of the rings is aromatic. In embodiments, the aryl may be fully aromatic, examples of which are phenyl, naphthyl, anthracenyl, acenaphthylenyl, azulenyl, fluorenyl, indenyl and pyrenyl. In embodiments, the aryl may also contain an aromatic ring in combination with a non-aromatic ring, examples of which are acenaphene, indene, and fluorene. In embodiments, the term includes both substituted and unsubstituted moieties. In embodiments, the aryl group can be substituted with any described moiety, including, but not limited to, one or more moieties selected from the group consisting of halo (fluoro, chloro, bromo, or iodo), alkyl, hydroxyl, amino, alkoxy, aryloxy, nitro, and cyano.

In embodiments, the term “heteroaryl” refers to a heterocyclyl group as defined above which is aromatic. In embodiments, the heteroaryl group may be attached to the main structure at any heteroatom or carbon atom which results in the creation of a stable compound.

In embodiments, examples of such heteroaryl groups include, but are not limited to: acridinyl, benzimidazolyl, benzindolyl, benzisoxazinyl, benzo[4,6]imidazo[1,2-a]pyridinyl, benzofuranyl, benzonaphthofuranyl, benzothiadiazolyl, benzothiazolyl, benzothiophenyl, benzotriazolyl, benzothiopyranyl, benzoxazinyl, benzoxazolyl, benzothiazolyl, β-carbolinyl, carbazolyl, cinnolinyl, dibenzofuranyl, furanyl, imidazolyl, imidazopyridinyl, imidazothiazolyl, indazolyl, indolizinyl, indolyl, isobenzothienyl, isoindolinyl, isoquinolinyl, isothiazolidinyl, isothiazolyl, naphthyridinyl, octahydroindolyl, octahydroisoindolyl, oxazolidinonyl, oxazolidinyl, oxazolopyridinyl, oxazolyl, isoxazolyl, oxiranyl, perimidinyl, phenanthridinyl, phenathrolinyl, phenarsazinyl, phenazinyl, phenothiazinyl, phenoxazinyl, phthalazinyl, pteridinyl, purinyl, pyrazinyl, pyrazolyl, pyridazinyl, pyridinyl, pyridopyridinyl, pyrimidinyl, pyrrolyl, quinazolinyl, quinolinyl, quinoxalinyl, tetrazolyl, thiadiazolyl, thiazolyl, thienyl, triazinyl, and triazolyl.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl, or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The term “heteroaralkyl” refers to a group of the formula —RₖR_{f} where Rₖ is an alkyl group as defined above and R_{f} is a heteroaryl group as defined herein. The alkyl group and the heteroaryl group may be optionally substituted as defined herein.

The term “azolyl” or “azoylyl” refers to a 5-membered heterocyclic or heteroaryl ring system containing at least one nitrogen atom. Exemplary azolyl rings include pyrazolyl, imidazolyl, oxazolyl, isoxazolyl, oxadiazolyl, thiazolyl, isothiazolyl, thiadiazolyl, diazolyl, and triazolyl.

The term “heterocyclylalkyl” refers to a group of the formula —RₖR_{g} wherein Rₖ is an alkyl group as defined above and R_{g} is a heterocyclyl group as defined herein, where the alkyl group Rₖ may attach at either the carbon atom or the heteroatom of the heterocyclyl group R_{g}. The alkyl group and the heterocyclyl group may be optionally substituted as defined herein.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

In embodiments, the term “alkylsulfonyl” refers to groups of the formula (SO₂)-alkyl, in which the sulfur atom is the point of attachment. In embodiments, alkylsulfonyl groups for use in the present invention may include C₁-C₆ alkylsulfonyl groups, which have from 1 to 6 carbon atoms. In embodiments, methylsulfonyl is one representative alkylsulfonyl group.

The term “alkylarylene” as an arylene moiety covalently bonded to an alkylene moiety (also referred to herein as an alkylene linker). In embodiments, the alkylarylene group has the formula:

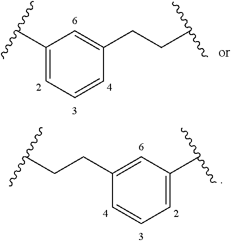 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃ —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —N₀₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si). In embodiments, the term “heteroatom” refers to any atom other than carbon, for example, N, O, or S.

A “substituent group,” as used herein, means a group selected from the following moieties:
(A) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, the term “substituent,” as used herein, refers to a molecular moiety that is covalently bonded to an atom within a molecule of interest. For example, a “ring substituent” may be a moiety such as a halogen, alkyl group, haloalkyl group or other group discussed herein that is covalently bonded to an atom (such as a carbon or nitrogen atom) that is a ring member.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂M alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and/or each substituted or unsubstituted heteroaryl is a substituted or unsubstituted 5 to 6 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ phenylene, and/or each substituted or unsubstituted heteroarylene is a substituted or unsubstituted 5 to 6 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

The term “optionally substituted” indicates that the referenced aryl or heterocyclyl or other group may be substituted at one or more substitutable positions by one or more groups independently selected from alkyl (for example lower alkyl), alkoxy (preferably lower alkoxy), nitro, monoalkylamino (for example with one to six carbons), dialkylamino (for example with one to six carbons), cyano, halo, haloalkyl (preferably trifluoromethyl), alkanoyl, aminocarbonyl, monoalkylaminocarbonyl, dialkylaminocarbonyl, alkyl amido (for example lower alkyl amido), alkoxyalkyl (for example a lower alkoxy and lower alkyl), alkoxycarbonyl (for example a lower alkoxycarbonyl), alkylcarbonyloxy (for example a lower alkylcarbonyloxy) and aryl (for example phenyl), said aryl being optionally substituted by halo, lower alkyl and lower alkoxy groups. Optional substitution is also indicated by the phrase “substituted with from 0 to X substituents,” where X is the maximum number of possible substituents. Certain optionally substituted groups are substituted with from 0 to 2, 3 or 4 independently selected substituents.

The term “independently” means that where more than one substituent is selected from a number of possible substituents, those substituents may be the same or different.

The term “alkoxycarbonyl” refers to a group having the formula —C(O)OR wherein R is any substituent forming a stable compound. In embodiments, R is alkyl, including lower alkyl.

The term “oxo” refers to the group ═O attached to a carbon atom.

A dash (“-”) that is not between two letters or symbols is used to indicate a point of attachment for a substituent. For example, —CONH₂ is attached through the carbon atom. In embodiments, the symbol “ [symbol] ” denotes the point of attachment of a chemical moiety to the remainder of a molecule or chemicaformula.

Unless stated otherwise specifically described in the specification, it is understood that the substitution can occur on any atom of the alkyl, alkenyl, alkynyl, cycloalkyl, heterocyclyl, aryl or heteroaryl group.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The terms “bind” and “bound” as used herein is used in accordance with its plain and ordinary meaning and refers to the association between atoms or molecules. The association can be direct or indirect. For example, bound atoms or molecules may be bound, e.g., by covalent bond, linker (e.g. a first linker or second linker), or non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like).

The term “capable of binding” as used herein refers to a moiety (e.g. a compound as described herein) that is able to measurably bind to a target (e.g., a NF-κB, a Toll-like receptor protein). In embodiments, where a moiety is capable of binding a target, the moiety is capable of binding with a Kd of less than about 10 μM, 5 μM, 1 μM, 500 nM, 250 nM, 100 nM, 75 nM, 50 nM, 25 nM, 15 nM, 10 nM, 5 nM, 1 nM, or about 0.1 nM.

As used herein, the term “conjugated” when referring to two moieties means the two moieties are bonded, wherein the bond or bonds connecting the two moieties may be covalent or non-covalent. In embodiments, the two moieties are covalently bonded to each other (e.g. directly or through a covalently bonded intermediary). In embodiments, the two moieties are non-covalently bonded (e.g. through ionic bond(s), van der waal's bond(s)/interactions, hydrogen bond(s), polar bond(s), or combinations or mixtures thereof).

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

The term “Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

As used herein and unless otherwise indicated, the term “hydrate” means a compound provided herein or a salt thereof, that further includes a stoichiometric or non-stoichiometeric amount of water bound by non-covalent intermolecular forces.

As used herein and unless otherwise indicated, the term “solvate” means a solvate formed from the association of one or more solvent molecules to a compound provided herein. The term “solvate” includes hydrates (e.g., mono-hydrate, dihydrate, trihydrate, tetrahydrate, and the like).

As used herein, “substantially pure” means sufficiently homogeneous to appear free of readily detectable impurities as determined by standard methods of analysis, such as thin layer chromatography (TLC), gel electrophoresis, high performance liquid chromatography (HPLC), and mass spectrometry (MS), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers. In such instances, further purification might increase the specific activity of the compound.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

Unless specifically stated otherwise, where a compound may assume alternative tautomeric, regioisomeric and/or stereoisomeric forms, all alternative isomers are intended to be encompassed within the scope of the claimed subject matter. For example, where a compound is described as having one of two tautomeric forms, it is intended that the both tautomers be encompassed herein. Thus, the compounds provided herein may be enantiomerically pure, or be stereoisomeric or diastereomeric mixtures.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers. In embodiments, it is to be understood that the compounds provided herein may contain chiral centers. In embodiments, such chiral centers may be of either the (R) or (S) configuration, or may be a mixture thereof.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ^{13C}- or ^{14C}-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (^{14C}). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the terms “bioconjugate” and “bioconjugate linker” refer to the resulting association between atoms or molecules of “bioconjugate reactive groups” or “bioconjugate reactive moieties”. The association can be direct or indirect. For example, a conjugate between a first bioconjugate reactive group (e.g., —NH₂, —C(O)OH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g. a first linker of second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions), and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g. a sulfhydryl). In embodiments, the first bioconjugate reactive group (e.g., haloacetyl moiety) is covalently attached to the second bioconjugate reactive group (e.g. a sulfhydryl). In embodiments, the first bioconjugate reactive group (e.g., pyridyl moiety) is covalently attached to the second bioconjugate reactive group (e.g. a sulfhydryl). In embodiments, the first bioconjugate reactive group (e.g., —N-hydroxysuccinimide moiety) is covalently attached to the second bioconjugate reactive group (e.g. an amine). In embodiments, the first bioconjugate reactive group (e.g., maleimide moiety) is covalently attached to the second bioconjugate reactive group (e.g. a sulfhydryl). In embodiments, the first bioconjugate reactive group (e.g., -sulfo-N-hydroxysuccinimide moiety) is covalently attached to the second bioconjugate reactive group (e.g. an amine).

Useful bioconjugate reactive moieties used for bioconjugate chemistries herein include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido or maleimide groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold, or react with maleimides;
(h) amine or sulfhydryl groups (e.g., present in cysteine), which can be, for example, acylated, alkylated or oxidized;
(i) alkenes, which can undergo, for example, cycloadditions, acylation, Michael addition, etc;
(j) epoxides, which can react with, for example, amines and hydroxyl compounds;
(k) phosphoramidites and other standard functional groups useful in nucleic acid synthesis;
(l) metal silicon oxide bonding; and
(m) metal bonding to reactive phosphorus groups (e.g. phosphines) to form, for example, phosphate diester bonds.
(n) azides coupled to alkynes using copper catalyzed cycloaddition click chemistry.
(o) biotin conjugate can react with avidin or strepavidin to form a avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13.A}, R^{13.B}, R^{13.C}, R^{13.D}, etc., wherein each of R^{13.A}, R^{13.B}, R^{13.C}, R^{13.D}, etc. is defined within the scope of the definition of R³ and optionally differently.

A “detectable agent” or “detectable moiety” is a composition, substance, element, or compound; or moiety thereof, detectable by appropriate means such as spectroscopic, photochemical, biochemical, immunochemical, chemical, magnetic resonance imaging, or other physical means. For example, useful detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g. fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. A detectable moiety is a monovalent detectable agent or a detectable agent capable of forming a bond with another composition.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, and ²²⁵Ac. Paramagnetic ions that may be used as additional imaging agents in accordance with the embodiments of the disclosure include, but are not limited to, ions of transition and lanthanide metals (e.g. metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb, and Lu.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or —CH₃). Likewise, for a linker variable (e.g., L¹, L², or L³ as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

The terms “disease” or “condition” refer to a state of being or health status of a patient or subject capable of being treated with the compounds or methods provided herein. The disease may be a cancer. The disease may be an infectious disease. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma.

As used herein, the term “cancer” refers to all types of cancer, neoplasm or malignant tumors found in mammals (e.g. humans), including leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cunateous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “cutaneous metastasis” or “skin metastasis” refer to secondary malignant cell growths in the skin, wherein the malignant cells originate from a primary cancer site (e.g., breast). In cutaneous metastasis, cancerous cells from a primary cancer site may migrate to the skin where they divide and cause lesions. Cutaneous metastasis may result from the migration of cancer cells from breast cancer tumors to the skin.

The term “visceral metastasis” refer to secondary malignant cell growths in the interal organs (e.g., heart, lungs, liver, pancreas, intestines) or body cavities (e.g., pleura, peritoneum), wherein the malignant cells originate from a primary cancer site (e.g., head and neck, liver, breast). In visceral metastasis, cancerous cells from a primary cancer site may migrate to the internal organs where they divide and cause lesions. Visceral metastasis may result from the migration of cancer cells from liver cancer tumors or head and neck tumors to internal organs.

The terms “treating” or “treatment” refer to any indicia of success in the therapy or amelioration of an injury, disease, pathology, or condition, including any objective or subjective parameter such as abatement; remission; diminishing of symptoms or making the injury, pathology, or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is no prophylactic treatment.

The term “prevent” refers to a decrease in the occurrence of disease symptoms in a patient. As indicated above, the prevention may be complete (no detectable symptoms) or partial, such that fewer symptoms are observed than would likely occur absent treatment.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found=effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to affect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” it is meant that a composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

An “anticancer agent” as used herein refers to a molecule (e.g. compound, peptide, protein, or nucleic acid) used to treat cancer through destruction or inhibition of cancer cells or tissues. Anticancer agents may be selective for certain cancers or certain tissues. In embodiments, anticancer agents herein may include epigenetic inhibitors and multi-kinase inhibitors.

“Anti-cancer agent” and “anticancer agent” are used in accordance with their plain ordinary meaning and refers to a composition (e.g. compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec.RTM.), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin I1 (including recombinant interleukin II, or rlL.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol.™ (i.e. paclitaxel), Taxotere.™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP—XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), LS-4559-P (Pharmacia, i.e. LS-4577), LS-4578 (Pharmacia, i.e. LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e. LY-355703), AC-7739 (Ajinomoto, i.e. AVE-8063A and CS-39.HCl), AC-7700 (Ajinomoto, i.e. AVE-8062, AVE-8062A, CS-39-L-Ser.HCl, and RPR-258062A), Vitilevuamide, Tubulysin A, Canadensol, Centaureidin (i.e. NSC-106969), T-138067 (Tularik, i.e. T-67, TL-138067 and TI-138067), COBRA-1 (Parker Hughes Institute, i.e. DDE-261 and WHI-261), H10 (Kansas State University), H16 (Kansas State University), Oncocidin A1 (i.e. BTO-956 and DIME), DDE-313 (Parker Hughes Institute), Fijianolide B, Laulimalide, SPA-2 (Parker Hughes Institute), SPA-1 (Parker Hughes Institute, i.e. SPIKET-P), 3-IAABU (Cytoskeleton/Mt. Sinai School of Medicine, i.e. MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e. MF-191), TMPN (Arizona State University), Vanadocene acetylacetonate, T-138026 (Tularik), Monsatrol, lnanocine (i.e. NSC-698666), 3-IAABE (Cytoskeleton/Mt. Sinai School of Medicine), A-204197 (Abbott), T-607 (Tuiarik, i.e. T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta Medica), D-68836 (Asta Medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guerin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), immunotherapy (e.g., cellular immunotherapy, antibody therapy, cytokine therapy, combination immunotherapy, etc.), radioimmunotherapy (e.g., anti-CD20 monoclonal antibody conjugated to ¹¹¹In, ⁹⁰Y, or ¹³¹I, etc.), immune checkpoint inhibitors (e.g., CTLA4 blockade, PD-1 inhibitors, PD-L¹ inhibitors, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like.

In embodiments, the term “anti-cancer agents” refers to anti-metabolites (e.g., 5-fluoro-uracil, methotrexate, fludarabine), antimicrotubule agents (e.g., vinca alkaloids such as vincristine, vinblastine; taxanes such as paclitaxel, docetaxel), alkylating agents (e.g., cyclophosphamide, melphalan, carmustine, nitrosoureas such as bischloroethylnitrosurea and hydroxyurea), platinum agents (e.g. cisplatin, carboplatin, oxaliplatin and satraplatin), anthracyclines (e.g., doxrubicin, daunorubicin), antitumor antibiotics (e.g., mitomycin, idarubicin, adriamycin, daunomycin), topoisomerase inhibitors (e.g., etoposide, camptothecins), anti-angiogenesis agents (e.g. Sutent® and Bevacizumab) or any other cytotoxic agents, (estramustine phosphate, prednimustine), hormones or hormone agonists, antagonists, partial agonists or partial antagonists, kinase inhibitors, and radiation treatment.

“Selective” or “selectivity” or the like of a compound refers to the compound's ability to discriminate between molecular targets.

“Specific”, “specifically”, “specificity”, or the like of a compound refers to the compound's ability to cause a particular action, such as inhibition, to a particular molecular target with minimal or no action to other proteins in the cell.

As used herein, the abbreviations for any protective groups, amino acids and other compounds, are, unless indicated otherwise, in accord with their common usage or recognized abbreviations including abbreviations found in prior art.

The term “a STING agonist molecule,” refers to a compound capable of binding to STING and activating STING. Activation of STING activity can include, for example, stimulation of inflammatory cytokines, including interferons, such as type 1 interferons, including IFN-a IFN-b, type 3 interferons, e.g., IFNy, or other proinflammatory molecules including but not limited to IP 10, TNF, ISG15, IL-6, CXCL9, CXCL10, CCL4, CXCL11, CCL5, CCL3, or CCL8. STING agonist activity can also include stimulation of TANK binding kinase (TBK) 1 phosphorylation, STING phosphorylation, interferon regulatory factor (IRF) activation (e.g., IRF3 activation), NFKB activation, STAT6 activation, secretion of interferon-y-inducible protein (IP-10), or other inflammatory proteins and cytokines.

An “inhibitor” refers to a compound (e.g. compounds described herein) that reduces activity when compared to a control, such as absence of the compound or a compound with known inactivity.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated, however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

As defined herein, the term “activation”, “activate”, “activating”, “activator” and the like in reference to a protein-inhibitor interaction means positively affecting (e.g. increasing) the activity or function of the protein relative to the activity or function of the protein in the absence of the activator. In embodiments, activation means positively affecting (e.g. increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein which is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein

The terms “agonist,” “activator,” “upregulator,” etc. refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In embodiments, inhibition means negatively affecting (e.g. decreasing) the concentration or levels of the protein relative to the concentration or level of the protein in the absence of the inhibitor. In embodiments, inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulator” refers to a composition that increases or decreases the level of a target molecule or the function of a target molecule or the physical state of the target of the molecule relative to the absence of the modulator.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The term “associated” or “associated with” in the context of a substance or substance activity or function associated with a disease (e.g. a protein associated disease, a cancer (e.g., cancer, or infectious disease)) means that the disease (e.g. cancer, inflammatory disease, autoimmune disease, or infectious disease) is caused by (in whole or in part), or a symptom of the disease is caused by (in whole or in part) the substance or substance activity or function. As used herein, what is described as being associated with a disease, if a causative agent, could be a target for treatment of the disease.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

In this disclosure, “comprises”, “comprising”, “containing”, “having”, and the like can have the meaning ascribed to them in U.S. Patent law and can mean “includes”. “including”. and the like. “Consisting essentially of or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

### II. COMPOUNDS

In an aspect is provided a compound having the formula:

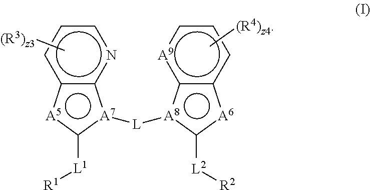

R¹ is independently substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).
R² is independently substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).
R³ is independently halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O) NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, —O—P(O)(OH)₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆).

In embodiments, R³ is independently halogen, —CF₃, —CHF₂, —CH₂F, —OCF₃, —OCH₂F, —OCHF₂, —CN, —N(O)ₘ₃, —SF₅, —N₃, —O—P(O)(OH)₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), or substituted or unsubstituted cycloalkyl (e.g., C₃-C₈, C₃-C₆, C₄-C₆, or C₅-C₆).

In embodiments, R³ is independently —SOₙ₃R^{3D}, —SOᵥ₃NR^{3A}R^{3B}, NR^{3C}NR^{3A}R^{3B}, —ONR^{3A}R^{3B}, —NHC(O)NR^{3C}NR^{3A}R^{3B}, —NHC(O)NR^{3A}R^{3B}, —NR^{3A}R^{3B}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, or —NR^{3A}OR^{3C}; wherein R^{3A}, R^{3B}, R^{3C}, and R^{3D} are independently hydrogen, substituted or unsubstituted alkyl (e.g., C₁-C₈, C₁-C₆, C₁-C₄, or C₁-C₂), substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{3A} and R^{3B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

In embodiments, R³ is independently —C(O)R^{3C} and R^{3C} is as described herein.

In embodiments, R³ is independently 1) halogen, —CF₃, —CHF₂, —CH₂F, —OCF₃, —OCH₂F, —OCHF₂, —CN, —N(O)ₘ₃, —SF₅, —N₃, —O—P(O)(OH)₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), or substituted or unsubstituted cycloalkyl (e.g., C₃-C₈, C₃-C₆, C₄-C₆, or C₅-C₆); 2) —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —NR^{3A}R^{3B}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, or —NR^{3A}OR^{3C}; wherein R^{3A}, R^{3B}, R^{3C}, and R^{3D} are independently hydrogen, substituted or unsubstituted alkyl (e.g., C₁-C₈, C₁-C₆, C₁-C₄, or C₁-C₂), substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{3A} and R^{3B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); or 3) —C(O)R^{3C}; wherein R^{3C} is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{3A} and R^{3B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

In embodiments, R³ is independently halogen, —CF₃, —CHF₂, —CH₂F, —OCF₃, —OCH₂F, —OCHF₂, —CN, —SOₙ₃R^{3D}, —SOᵥ₃NR^{3A}R^{3B}, NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O) NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, —O—P(O)(OH)₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆).

In embodiments, R^{3A}, R^{3B}, R^{3C}, and R^{3D} are independently hydrogen, substituted or unsubstituted alkyl (e.g., C₁-C₈, C₁-C₆, C₁-C₄, or C₁-C₂), substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{3A} and R^{3B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).
z3 is 0, 1, 2, or 3.
X³ is independently —F, —Cl, —Br, or —I.
m3 and v3 are independently 1 or 2.
n3 is independently an integer from 0 to 4.
R^{3A}, R^{3B}, R^{3C}, and R^{3D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{3A} and R^{3B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).
R⁴ is independently halogen, —CX⁴₃, —CHX⁴₂, —CH₂X⁴, —OCX⁴₃, —OCH₂X⁴, —OCHX⁴₂, —CN, —SO₄R^{4D}, —SOᵥ₄NR^{4A}R^{4B}, —N^{4C}NR^{4A}R^{4B}, —ONR^{4A}R^{4B}, —NHC(O)NR^{4C}NR^{4A}R^{4B}, —NHC(O)NR^{4A}R^{4B}, —N(O)ₘ₄, —NR^{4A}R^{4B}, —C(O)R^{4C}, —C(O)—OR^{4C}, —C(O) N^{4A}R^{4B}, —OR^{4D}, —N^{4A}SO₂R^{4D}, —NR^{4A}C(O)R^{4C}, —NR^{4A}C(O)ORC, —NR^{4A}OR^{4C}, —SF₅, —N₃, —O—P(O)(OH)₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), or substituted or unsubstituted cycloalkyl (e.g., C₃-C₈, C₃-C₆, C₄-C₆, or C₅-C₆).

In embodiments, R⁴ is independently halogen, —CF₃, —CHF₂, —CH₂F, —OCF₃, —OCH₂F, —OCHF₂, —CN, —N(O)ₘ₄, —SF₅, —N₃, —O—P(O)(OH)₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), or substituted or unsubstituted cycloalkyl (e.g., C₃-C₈, C₃-C₆, C₄-C₆, or C₅-C₆).

In embodiments, R⁴ is independently —SOₙ₄R^{4D}, —SOᵥ₄NR^{4A}R^{4B}, —N^{4C}NR^{4A}R^{4B}, —ONR^{4A}R^{4B}, —NHC(O)NR^{4C}NR^{4A}R^{4B}, —NHC(O)NR^{4A}R^{4B}, —N^{4A}R^{4B}, C(O)—OR^{4C}, —C(O)NR^{4A}R^{4B}, —OR^{4D}, —NR^{4A}SO₂R^{4D}, —NR^{4A}C(O)RC, —NR^{4A}C(O)OR^{4C}, or —NR^{4A}OR^{4C}; wherein R^{4A}, R^{4B}, R^{4C}, and R^{4D} are independently hydrogen, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{4A} and R^{4B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

In embodiments, R⁴ is independently —C(O)R^{4C} and R^{4C} is as described herein.

In embodiments, R⁴ is independently 1) halogen, —CF₃, —CHF₂, —CH₂F, —OCF₃, —OCH₂F, —OCHF₂, —CN, —N(O)ₘ₄, —SF₅, —N₃, —O—P(O)(OH)₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), or substituted or unsubstituted cycloalkyl (e.g., C₃-C₈, C₃-C₆, C₄-C₆, or C₅-C₆); 2) —SO₄R^{4D}, —SOᵥ₄NR^{4A}R^{4B}, —N^{4C}NR^{4A}R^{4B}, —ONR^{4A}R^{4B}, —NHC(O)NR^{4C}NR^{4A}R^{4B}, —NHC(O)NR^{4A}R^{4B}, —N^{4A}R^{4B}, C(O)—OR^{4C}, —C(O)NR^{4A}R^{4B}, —OR^{4D}, —NR^{4A}SO₂R^{4D}, —NR^{4A}C(O)RC, —NR^{4A}C(O)OR^{4C}, or —NR^{4A}OR^{4C}; wherein R^{4A}, R^{4B}, R^{4C}, and R^{4D} are independently hydrogen, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{4A} and R^{4B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); or 3) —C(O)R^{4C} wherein R^{4C} is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{4A} and R^{4B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

In embodiments, R⁴ is independently halogen, —CF₃, —CHF₂, —CH₂F, —OCF₃, —OCH₂F, —OCHF₂, —CN, —SOₙ₄R^{4D}, —SOᵥ₄NR^{4A}R^{4B}, —N^{4C}NR^{4A}R^{4B}, —ONR^{4A}R^{4B}, —NHC(O)NR^{4C}NR^{4A}R^{4B}, —NHC(O)NR^{4A}R^{4B}, —N(O)ₘ₄, —N^{4A}R^{4B}, C(O)R^{4C}, —C(O)—OR^{4C}, —C(O) N^{4A}R^{4B}, —OR, —N^{4A}SO₂R^{4D}, —NR^{4A}C(O)R^{4C}, —NR^{4A}C(O)OR, —NR^{4A}OR^{4C}, —SF₅, —N₃, —O—P(O)(OH)₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), or substituted or unsubstituted cycloalkyl (e.g., C₃-C₈, C₃-C₆, C₄-C₆, or C₅-C₆).
z4 is 0, 1, 2, or 3.
X⁴ is independently —F, —Cl, —Br, or —I.
m4 and v4 are independently 1 or 2.
n4 is independently an integer from 0 to 4.
R^{4A}, R^{4B}, R^{4C}, and R^{4D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{4A} and R^{4B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

In embodiments, R^{4A}, R^{4B}, R^{4C}, and R^{4D} are independently hydrogen, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{4A} and R^{4B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).
L is -L³-L⁴-L⁵-.
L³ and L⁵ are independently a bond, substituted or unsubstituted alkylene (e.g., C₁-C₁₀, C₁-C₈, C₁-C₆, C₁-C₄, C₁-C₂, C₂-C₁₀, C₂-C₈, C₂-C₆, or C₂-C₄,), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₁₀, C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).
L⁴ is independently a bond, —N(R^{L4})—, —O—, —S—, —SO₂—, —C(O)—, —C(O)N(R^{L4})—, —N(R^{L4})C(O)—, —N(R^{L4})C(O)NH—, —NHC(O)N(R^{L4})—, —C(O)O—, —OC(O)—, —SO₂N(R^{L4})—, —N(R^{L4})SO₂—, substituted or unsubstituted alkylene (e.g., C₁-C₁₀, C₁-C₈, C₁-C₆, C₁-C₄, C₁-C₂, C₂-C₁₀, C₂-C₈, C₂-C₆, or C₂-C₄,), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₁₀, C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).
R^{L4} is independently hydrogen, COR^{L4A}, CO₂H, CO₂R^{L4A}, SOR^{L4A}, SO₂R^{L4A}, CONH₂, CONR^{L4A}R^{L4B}, SO₂NH₂, SO₂NR^{L4A}R^{L4B}, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R^{L4} is independently hydrogen, COR^{L4A}, CO₂H, CO₂R^{L4A}, SOR^{L4A}, SO₂R^{L4A}, CONH₂, CONR^{L4A}R^{L4B}, SO₂NH₂, SO₂NR^{L4A}R^{L4B}, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R^{L4} is independently 1) hydrogen, CO₂H, CO₂R^{L4A}, SOR^{L4A}, SO₂R^{L4A}, CONH₂, CONR^{L4A}R^{L4B}, SO₂NH₂, SO₂NR^{L4A}R^{L4B}, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); wherein R^{L4A} and R^{L4B} are independently hydrogen, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{L4A} and R^{L4B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); or 2) COR^{L4A}; wherein R^{L4A} is independently

hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).
R^{L4A} and R^{L4B} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{L4A} and R^{L4B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

In embodiments, R^{L4A} and R^{L4B} are independently hydrogen, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{L4A} and R^{L4B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

In embodiments, R^{L4A} and R^{L4B} are independently hydrogen, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{L4A} and R^{L4B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

In embodiments, R^{L4A} and R^{L4B} are independently hydrogen, —CF₃, —CHF₂, —CH₂F, —CN, —OH, —NH₂, —COOH, —CONH₂, —OCF₃, —OCHF₂, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{L4A} and R^{L4B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).
L¹ is independently=NC(O)—, —C(O)N(R^{L1})—, or —N(R^{L1})C(O)—.
R^{L1} is independently hydrogen, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).
L² is independently=NC(O)—, —C(O)N(R^{L2})—, or —N(R^{L2})C(O)—.
R^{L2} is independently hydrogen, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).
A⁵ is independently O, S, N, NR⁵, or CR⁵.
R⁵ is independently hydrogen, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).
A⁶ is independently O, S, N, NR⁶, or CR⁶.
R⁶ is independently hydrogen, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).
A⁷ is independently N or CR⁷.
R⁷ is independently hydrogen, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).
A⁸ is independently N or CR⁸.
R⁸ is independently hydrogen, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).
A⁹ is independently N or CR⁹.
R⁹ is independently hydrogen, halogen, —CX⁹₃, —CHX⁹₂, —CH₂X⁹, —OCX⁹₃, —OCH₂X⁹, —OCHX⁹₂, —CN, —SOₙ₉R^{9D}, —SOᵥ₉NR^{9A}R^{9B}, —NR^{9C}NR^{9A}R^{9B}, —NHC(O)NR^{9C}NR^{9A}R^{9B}, —NHC(O)NR^{9A}R^{9B}, —N(O)ₘ₉, —NR^{9A}R^{9B}, —C(O)R^{9C}, —OC(O)R^{9C}, —C(O)—OR^{9C}, —OC(O)—OR^{9C}, —C(O)NR^{9A}R^{9B}, —OC(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, —SF₅, —N₃, O—P(O)(OR^{9A})₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R⁹ is independently hydrogen, halogen, —CF₃, —CHF₂, —CH₂F, —OCF₃, —OCH₂F, —OCHF₂, —CN, —SOₙ₉R^{9D}, —SOᵥ₉NR^{9A}R^{9B}, —NR^{9C}NR^{9A}R^{9B}, —ONR^{9A}R^{9B}, —NHC(O)NR^{9C}NR^{9A}R^{9B}, —NHC(O)NR^{9A}R^{9B}, —N(O)ₘ₉, —NR^{9A}R^{9B}, —C(O)R^{9C}, —OC(O)R^{9C}, —C(O)—OR^{9C}, —OC(O)—OR^{9C}, —C(O)NR^{9A}R^{9B}, —OC(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, —SF₅, —N₃, O—P(O)(OR^{9A})₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R⁹ is independently 1) hydrogen, halogen, —CF₃, —CHF₂, —CH₂F, —OCF₃, —OCH₂F, —OCHF₂, —CN, —SOₙ₉R^{9D}, —SOᵥ₉NR^{9A}R^{9B}, —NR^{9C}NR^{9A}R^{9B}, —ONR^{9A}R^{9B}, —NHC(O)NR^{9C}NR^{9A}R^{9B}, —NHC(O)NR^{9A}R^{9B}, —N(O)ₘ₉, —NR^{9A}R^{9B}, —C(O)—OR^{9C}, —OC(O)—OR^{9C}, —C(O)NR^{9A}R^{9B}, —OC(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A} OR^{9C}, —SF₅, —N₃, O—P(O)(OR^{9A})₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); wherein R^{9A}, R^{9B}, R^{9C}, and R^{9D} are independently hydrogen, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{9A} and R^{9B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); or 2) —C(O)R^{9C} or —OC(O)R^{9C}; wherein R^{9C} is independently hydrogen, —CCl₃, —CBr₃, —CF₃, —CI₃, —CHCl₂, —CHBr₂, —CHF₂, —CHI₂, —CH₂C₁, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂C₁, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).
z9 is 0, 1, 2, or 3.
X⁹ is independently —F, —Cl, —Br, or —I.
m9 and v9 are independently 1 or 2.
n9 is independently an integer from 0 to 4.
R^{9A}, R^{9B}, R^{9C}, and R^{9D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{9A} and R^{9B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

In embodiments, R^{9A}, R^{9B}, R^{9C}, and R^{9D} are independently hydrogen, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{9A} and R^{9B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

In embodiments, R¹ is independently R¹⁰-substituted or unsubstituted C₁-C₂₀ saturated alkyl, R¹⁰-substituted or unsubstituted C₂-C₂₀ alkenyl, R¹⁰-substituted or unsubstituted C₂-C₂₀ alkynyl, R¹⁰-substituted or unsubstituted C₃-C₁₀ cycloalkyl, R¹⁰-substituted or unsubstituted 4-10 membered heterocycloalkyl, R¹⁰-substituted or unsubstituted 6-10 membered aryl, or R¹⁰-substituted or unsubstituted 5-10 membered heteroaryl.
R¹⁰ is independently halogen, —CN, —CX¹⁰₃, —CHX¹⁰₂, —CH₂X₁₀, —OR^{10B}, —OC(O)R^{10B}, —OC(O)NR^{10A}R^{10B}, —C(O)—OR^{10B}, —C(O)NR^{10A}R^{10B}, —SOR^{10B}, —SO₂R^{10B}, —SO₂NR^{10A}R^{10B}, —O—P(O)(OR^{10A})₂, —NR^{10A}R^{10B}, —NR^{10A}C(O)R^{10B}, —NR^{10A}SOR^{10B}, —NR^{10A}C(O)OR^{10B}, —NR^{10A}SO₂R^{10B}, —C(O)R^{10B}, -(unsubstituted alkyl)-O—P(O)(OR^{10A})₂, -(unsubstituted alkoxy)-O—P(O)(OR^{10A})₂, —OCX¹⁰₃, —OCH₂X¹⁰, —OCHX¹⁰₂, —SR^{10B}, —NHNR^{10A}R^{10B}, —NR^{10A}R^{10B}, —NHC(O)NHNR^{10A}R^{10B}, —NHC(O)NR^{10A}R^{10B}, —N(O)₂, —NR^{10A}OR^{10B}, —SF₅, —N₃, R¹¹-substituted or unsubstituted C₁-C₆ saturated alkyl (e.g., C₁-C₆, C₁-C₄, or C₁-C₂), R¹-substituted or unsubstituted C₂-C₆ alkenyl (e.g., C₂-C₆, C₂-C₄, or C₂), R¹¹-substituted or unsubstituted C₂-C₆ alkynyl (e.g., C₂-C₆, C₂-C₄, or C₂), R¹¹-substituted or unsubstituted 2 to 8 membered heteroalkyl (e.g., 2 to 8 membered, 2 to 6 membered, 4 to 6 membered, 2 to 3 membered, or 4 to 5 membered), R¹¹-substituted or unsubstituted cycloalkyl (e.g., C₃-C₈, C₃-C₆, C₄-C₆, or C₅-C₆), R¹¹-substituted or unsubstituted heterocycloalkyl (e.g., 3 to 8 membered, 3 to 6 membered, 4 to 6 membered, 4 to 5 membered, or 5 to 6 membered), R¹¹-substituted or unsubstituted aryl (e.g., C₆-C₁₀ or phenyl), or R¹¹-substituted or unsubstituted heteroaryl (e.g., 5 to 10 membered, 5 to 9 membered, or 5 to 6 membered); R^{10A} and R^{10B} substituents bonded to the same nitrogen atom may optionally be joined to form a R¹¹-substituted or unsubstituted heterocycloalkyl or R¹¹-substituted or unsubstituted heteroaryl. X¹⁰ is independently —F, —Cl, —Br, or —I.

In embodiments, R¹⁰ is independently halogen, —CN, —CF₃, —CHF₂, —CH₂F, —OR^{10B}, —OC(O)R^{10B}, —OC(O)NR^{10A}R^{10B}, —C(O)—OR^{10B}, —C(O)NR^{10A}R^{10B}, —SOR^{10B}, —SO₂R^{10B}, —SO₂NR^{10A}R^{10B}, —O—P(O)(OR^{10A})₂, —NR^{10A}R^{10B}, —NR^{10A}C(O)R^{10B}, —NR^{10A}SOR^{10B}, —NR^{10A}C(O)OR^{10B}, —NR^{10A}SO₂R^{10B}, —C(O)R¹⁰, -(unsubstituted alkyl)-O—P(O)(OR^{10A})₂, -(unsubstituted alkoxy)-O—P(O)(OR^{10A})₂, —OCF₃, —OCH₂F, —OCHF₂, —SR^{10B}, —NHNR^{10A}R^{10B}, —ONR^{10A}R^{10B}, —NHC(O)NHNR^{10A}R^{10B}, —NHC(O)NR^{10A}R^{10B}, —N(O)₂, —NR^{10A}OR^{10B}, —SF₅, —N₃, R¹¹-substituted or unsubstituted C₁-C₆ saturated alkyl (e.g., C₁-C₆, C₁-C₄, or C₁-C₂), R¹¹-substituted or unsubstituted C₂-C₆ alkenyl (e.g., C₂-C₆, C₂-C₄, or C₂), R¹¹-substituted or unsubstituted C₂-C₆ alkynyl (e.g., C₂-C₆, C₂-C₄, or C₂), R¹¹-substituted or unsubstituted 2 to 8 membered heteroalkyl (e.g., 2 to 8 membered, 2 to 6 membered, 4 to 6 membered, 2 to 3 membered, or 4 to 5 membered), R¹¹-substituted or unsubstituted cycloalkyl (e.g., C₃-C₈, C₃-C₆, C₄-C₆, or C₅-C₆), R¹¹-substituted or unsubstituted heterocycloalkyl (e.g., 3 to 8 membered, 3 to 6 membered, 4 to 6 membered, 4 to 5 membered, or 5 to 6 membered), R¹¹-substituted or unsubstituted aryl (e.g., C₆-C₁₀ or phenyl), or R¹¹-substituted or unsubstituted heteroaryl (e.g., 5 to 10 membered, 5 to 9 membered, or 5 to 6 membered).

In embodiments, R¹⁰ is independently 1) halogen, —CN, —CF₃, —CHF₂, —CH₂F, —OR^{10B}, —OC(O)NR^{10A}R^{10B}, —C(O)—OR^{10B}, —C(O)NR^{10A}R^{10B}, —SOR^{10B}, —SO₂R^{10B}, —SO₂NR^{10A}R^{10B}, —O—P(O)(OR^{10A})₂, —NR^{10A}R^{10B}, —NR^{10A}SOR^{10B}, —NR^{10A}C(O)OR^{10B}, —NR^{10A}SO₂R^{10B}, -(unsubstituted alkyl)-O—P(O)(OR^{10A})₂, -(unsubstituted alkoxy)-O—P(O)(OR^{10A})₂, —OCF₃, —OCH₂F, —OCHF₂, —SR^{10B}, —NHNR^{10A}R^{10B}, —ONR^{10A}R^{10B}, —NHC(O)NHNR^{10A}R^{10B}, —NHC(O)NR^{10A}R^{10B}, —N(O)₂, —NR^{10A}OR^{10B}, —SF₅, —N₃, R¹¹-substituted or unsubstituted C₁-C₆ saturated alkyl (e.g., C₁-C₆, C₁-C₄, or C₁-C₂), R¹¹-substituted or unsubstituted C₂-C₆ alkenyl (e.g., C₂-C₆, C₂-C₄, or C₂), R¹¹-substituted or unsubstituted C₂-C₆ alkynyl (e.g., C₂-C₆, C₂-C₄, or C₂), R¹¹-substituted or unsubstituted 2 to 8 membered heteroalkyl (e.g., 2 to 8 membered, 2 to 6 membered, 4 to 6 membered, 2 to 3 membered, or 4 to 5 membered), R¹¹-substituted or unsubstituted cycloalkyl (e.g., C₃-C₈, C₃-C₆, C₄-C₆, or C₅-C₆), R¹¹-substituted or unsubstituted heterocycloalkyl (e.g., 3 to 8 membered, 3 to 6 membered, 4 to 6 membered, 4 to 5 membered, or 5 to 6 membered), R¹¹-substituted or unsubstituted aryl (e.g., C₆-C₁₀ or phenyl), or R¹¹-substituted or unsubstituted heteroaryl (e.g., 5 to 10 membered, 5 to 9 membered, or 5 to 6 membered); wherein R^{10A} and R^{10B} are independently hydrogen, R¹¹-substituted or unsubstituted saturated alkyl (e.g., C₁-C₆, C₁-C₄, or C₁-C₂), R¹¹-substituted or unsubstituted alkenyl (e.g., C₂-C₆, C₂-C₄, or C₂), R¹¹-substituted or unsubstituted alkynyl (e.g., C₂-C₆, C₂-C₄, or C₂), R¹¹-substituted or unsubstituted heteroalkyl (e.g., 2 to 8 membered, 2 to 6 membered, 4 to 6 membered, 2 to 3 membered, or 4 to 5 membered), R¹¹-substituted or unsubstituted cycloalkyl (e.g., C₃-C₈, C₃-C₆, C₄-C₆, or C₅-C₆), R¹¹-substituted or unsubstituted heterocycloalkyl (e.g., 3 to 8 membered, 3 to 6 membered, 4 to 6 membered, 4 to 5 membered, or 5 to 6 membered), R¹¹-substituted or unsubstituted aryl (e.g., C₆-C₁₀ or phenyl), or R¹¹-substituted or unsubstituted heteroaryl (e.g., 5 to 10 membered, 5 to 9 membered, or 5 to 6 membered); or 2) —OC(O)R^{10B}, —NR^{10A}C(O)R^{10B}, or —C(O)R^{10B}; wherein R^{10A} is independently hydrogen, R¹¹-substituted or unsubstituted saturated alkyl (e.g., C₁-C₆, C₁-C₄, or C₁-C₂), R¹¹-substituted or unsubstituted alkenyl (e.g., C₂-C₆, C₂-C₄, or C₂), R¹¹-substituted or unsubstituted alkynyl (e.g., C₂-C₆, C₂-C₄, or C₂), R¹¹-substituted or unsubstituted heteroalkyl (e.g., 2 to 8 membered, 2 to 6 membered, 4 to 6 membered, 2 to 3 membered, or 4 to 5 membered), R¹¹-substituted or unsubstituted cycloalkyl (e.g., C₃-C₈, C₃-C₆, C₄-C₆, or C₅-C₆), R¹¹-substituted or unsubstituted heterocycloalkyl (e.g., 3 to 8 membered, 3 to 6 membered, 4 to 6 membered, 4 to 5 membered, or 5 to 6 membered), R¹¹-substituted or unsubstituted aryl (e.g., C₆-C₁₀ or phenyl), or R¹¹-substituted or unsubstituted heteroaryl (e.g., 5 to 10 membered, 5 to 9 membered, or 5 to 6 membered); and R^{10B} is independently hydrogen, —CCl₃, —CBr₃, —CF₃, —CI₃, —CHCl₂, —CHBr₂, —CHF₂, —CHI₂, —CH₂C₁, —CH₂Br, —CH₂F, —CH₂L, —CN, —OH, —COH, —COOH, —CONH₂, —OCCl₃, —OCF₃, —OCBr₃, —OCI₃, —OCHCl₂, —OCHBr₂, —OCHI₂, —OCHF₂, —OCH₂C₁, —OCH₂Br, —OCH₂I, —OCH₂F, R″-substituted or unsubstituted saturated alkyl (e.g., C₁-C₆, C₁-C₄, or C₁-C₂), R¹¹-substituted or unsubstituted alkenyl (e.g., C₂-C₆, C₂-C₄, or C₂), R¹¹-substituted or unsubstituted alkynyl (e.g., C₂-C₆, C₂-C₄, or C₂), R¹-substituted or unsubstituted heteroalkyl (e.g., 2 to 8 membered, 2 to 6 membered, 4 to 6 membered, 2 to 3 membered, or 4 to 5 membered), R¹-substituted or unsubstituted cycloalkyl (e.g., C₃-C₈, C₃-C₆, C₄-C₆, or C₅-C₆), R¹-substituted or unsubstituted heterocycloalkyl (e.g., 3 to 8 membered, 3 to 6 membered, 4 to 6 membered, 4 to 5 membered, or 5 to 6 membered), R¹¹-substituted or unsubstituted aryl (e.g., C₆-C₁₀ or phenyl), or R¹¹-substituted or unsubstituted heteroaryl (e.g., 5 to 10 membered, 5 to 9 membered, or 5 to 6 membered).
R^{10A} and R^{10B} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —COH, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R¹¹-substituted or unsubstituted saturated alkyl (e.g., C₁-C₆, C₁-C₄, or C₁-C₂), R¹-substituted or unsubstituted alkenyl (e.g., C₂-C₆, C₂-C₄, or C₂), R¹-substituted or unsubstituted alkynyl (e.g., C₂-C₆, C₂-C₄, or C₂), R¹-substituted or unsubstituted heteroalkyl (e.g., 2 to 8 membered, 2 to 6 membered, 4 to 6 membered, 2 to 3 membered, or 4 to 5 membered), R¹-substituted or unsubstituted cycloalkyl (e.g., C₃-C₈, C₃-C₆, C₄-C₆, or C₅-C₆), R¹-substituted or unsubstituted heterocycloalkyl (e.g., 3 to 8 membered, 3 to 6 membered, 4 to 6 membered, 4 to 5 membered, or 5 to 6 membered), R¹¹-substituted or unsubstituted aryl (e.g., C₆-C₁₀ or phenyl), or R¹¹-substituted or unsubstituted heteroaryl (e.g., 5 to 10 membered, 5 to 9 membered, or 5 to 6 membered). In embodiments, R^{10A} and R^{10B} substituents bonded to the same nitrogen atom may optionally be joined to form a R¹-substituted or unsubstituted heterocycloalkyl (e.g., 3 to 8 membered, 3 to 6 membered, 4 to 6 membered, 4 to 5 membered, or 5 to 6 membered) or R¹¹-substituted or unsubstituted heteroaryl (e.g., 5 to 10 membered, 5 to 9 membered, or 5 to 6 membered).

In embodiments, R^{10A} and R^{10B} are independently hydrogen, R¹¹-substituted or unsubstituted saturated alkyl (e.g., C₁-C₆, C₁-C₄, or C₁-C₂), R¹-substituted or unsubstituted alkenyl (e.g., C₂-C₆, C₂-C₄, or C₂), R¹-substituted or unsubstituted alkynyl (e.g., C₂-C₆, C₂-C₄, or C₂), R¹-substituted or unsubstituted heteroalkyl (e.g., 2 to 8 membered, 2 to 6 membered, 4 to 6 membered, 2 to 3 membered, or 4 to 5 membered), R¹-substituted or unsubstituted cycloalkyl (e.g., C₃-C₈, C₃-C₆, C₄-C₆, or C₅-C₆), R¹¹-substituted or unsubstituted heterocycloalkyl (e.g., 3 to 8 membered, 3 to 6 membered, 4 to 6 membered, 4 to 5 membered, or 5 to 6 membered), R″-substituted or unsubstituted aryl (e.g., C₆-C₁₀ or phenyl), or R¹¹-substituted or unsubstituted heteroaryl (e.g., 5 to 10 membered, 5 to 9 membered, or 5 to 6 membered).

In embodiments, R^{10A} and R^{10B} are independently hydrogen, —CF₃, —CHF₂, —CH₂F, —CN, —OH, —COH, —COOH, —CONH₂, —OCF₃, —OCHF₂, —OCH₂F, R¹¹-substituted or unsubstituted saturated alkyl (e.g., C₁-C₆, C₁-C₄, or C₁-C₂), R¹¹-substituted or unsubstituted alkenyl (e.g., C₂-C₆, C₂-C₄, or C₂), R¹¹-substituted or unsubstituted alkynyl (e.g., C₂-C₆, C₂-C₄, or C₂), R¹-substituted or unsubstituted heteroalkyl (e.g., 2 to 8 membered, 2 to 6 membered, 4 to 6 membered, 2 to 3 membered, or 4 to 5 membered), R¹¹-substituted or unsubstituted cycloalkyl (e.g., C₃-C₈, C₃-C₆, C₄-C₆, or C₅-C₆), R¹-substituted or unsubstituted heterocycloalkyl (e.g., 3 to 8 membered, 3 to 6 membered, 4 to 6 membered, 4 to 5 membered, or 5 to 6 membered), R¹¹-substituted or unsubstituted aryl (e.g., C₆-C₁₀ or phenyl), or R¹¹-substituted or unsubstituted heteroaryl (e.g., 5 to 10 membered, 5 to 9 membered, or 5 to 6 membered).

In embodiments, R^{10A} and R^{10B} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —COH, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R¹¹-substituted or unsubstituted C₁-C₁₀ saturated alkyl (e.g., C₁-C₆, C₁-C₄, or C₁-C₂), R¹¹-substituted or unsubstituted C₂-C₁₀ alkenyl (e.g., C₂-C₆, C₂-C₄, or C₂), R¹¹-substituted or unsubstituted C₂-C₁₀ alkynyl (e.g., C₂-C₆, C₂-C₄, or C₂), R¹¹-substituted or unsubstituted 2 to 10 membered heteroalkyl (e.g., 2 to 8 membered, 2 to 6 membered, 4 to 6 membered, 2 to 3 membered, or 4 to 5 membered), R¹¹-substituted or unsubstituted cycloalkyl (e.g., C₃-C₈, C₃-C₆, C₄-C₆, or C₅-C₆), R¹-substituted or unsubstituted heterocycloalkyl (e.g., 3 to 8 membered, 3 to 6 membered, 4 to 6 membered, 4 to 5 membered, or 5 to 6 membered), R¹-substituted or unsubstituted aryl (e.g., C₆-C₁₀ or phenyl), or R¹¹-substituted or unsubstituted heteroaryl (e.g., 5 to 10 membered, 5 to 9 membered, or 5 to 6 membered).

R¹¹ is independently oxo, halogen, —CCl₃, —CBr₃, —CF₃, —CI₃, —CHCl₂, —CHBr₂, —CHF₂, —CHI₂, -CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, R¹²-substituted or unsubstituted saturated alkyl (e.g., C₁-C₈, C₁-C₆, C₁-C₄, or C₁-C₂), R¹²-substituted or unsubstituted alkenyl (e.g., C₂-C₈, C₂-C₆, C₂-C₄, or C₂), R¹²-substituted or unsubstituted alkynyl (e.g., C₂-C₈, C₂-C₆, C₂-C₄, or C₂), R¹²-substituted or unsubstituted heteroalkyl (e.g., 2 to 12 membered, 2 to 6 membered, 4 to 6 membered, 2 to 3 membered, or 4 to 5 membered), R¹²-substituted or unsubstituted cycloalkyl (e.g., C₃-C₈, C₃-C₆, C₄-C₆, or C₅-C₆), R¹²-substituted or unsubstituted heterocycloalkyl (e.g., 3 to 15 membered, 3 to 6 membered, 4 to 6 membered, 4 to 5 membered, or 5 to 6 membered), R¹²-substituted or unsubstituted aryl (e.g., C₆-C₁₀ or phenyl), or R¹²-substituted or unsubstituted heteroaryl (e.g., 5 to 10 membered, 5 to 9 membered, or 5 to 6 membered).

In embodiments, R¹¹ is independently oxo, halogen, —CF₃, —CHF₂, —CH₂F, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSO₂H, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, —OCH₂F, —SF₅, —N₃, —O—P(O)(OH)₂, R¹²-substituted or unsubstituted saturated alkyl (e.g., C₁-C₈, C₁-C₆, C₁-C₄, or C₁-C₂), R¹²-substituted or unsubstituted alkenyl (e.g., C₂-C₈, C₂-C₆, C₂-C₄, or C₂), R¹²-substituted or unsubstituted alkynyl (e.g., C₂-C₈, C₂-C₆, C₂-C₄, or C₂), R¹²-substituted or unsubstituted heteroalkyl (e.g., 2 to 8 membered, 2 to 6 membered, 4 to 6 membered, 2 to 3 membered, or 4 to 5 membered), R¹²-substituted or unsubstituted cycloalkyl (e.g., C₃-C₈, C₃-C₆, C₄-C₆, or C₅-C₆), R¹²-substituted or unsubstituted heterocycloalkyl (e.g., 3 to 8 membered, 3 to 6 membered, 4 to 6 membered, 4 to 5 membered, or 5 to 6 membered), R¹²-substituted or unsubstituted aryl (e.g., C₆-C₁₀ or phenyl), or R¹²-substituted or unsubstituted heteroaryl (e.g., 5 to 10 membered, 5 to 9 membered, or 5 to 6 membered).

In embodiments, R¹¹ is independently oxo, halogen, —CCl₃, —CBr₃, —CF₃, —CI₃, —CHCl₂, —CHBr₂, —CHF₂, —CHI₂, -CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, R¹²-substituted or unsubstituted C₁-C₄ saturated alkyl (e.g., C₁-C₄, or C₁-C₂), R¹²-substituted or unsubstituted C₂-C₄ alkenyl (e.g., C₂-C₄, or C₂), R¹²-substituted or unsubstituted C₂-C₄ alkynyl (e.g., C₂-C₄, or C₂), R¹²-substituted or unsubstituted 2 to 12 membered heteroalkyl (e.g., 2 to 8 membered, 2 to 6 membered, 4 to 6 membered, 2 to 3 membered, or 4 to 5 membered), R¹²-substituted or unsubstituted C₃-C₆ cycloalkyl (e.g., C₃-C₆, C₄-C₆, or C₅-C₆), R¹²-substituted or unsubstituted 3 to 15 membered heterocycloalkyl (e.g., 3 to 6 membered, 4 to 6 membered, 4 to 5 membered, or 5 to 6 membered), R¹²-substituted or unsubstituted phenyl, or R¹²-substituted or unsubstituted 5 to 6 membered heteroaryl.
R¹² is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, R¹² is independently oxo, halogen, —CF₃, —CHF₂, —CH₂F, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSO₂H, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, —OCH₂F, —SF₅, —N₃, —O—P(O)(OH)₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, R² is independently R²⁰-substituted or unsubstituted C₁-C₂₀ saturated alkyl, R²⁰-substituted or unsubstituted C₂-C₂₀ alkenyl, R²⁰-substituted or unsubstituted C₂-C₂₀ alkynyl, R²⁰-substituted or unsubstituted C₃-C₁₀ cycloalkyl, R²⁰-substituted or unsubstituted 4-10 membered heterocycloalkyl, R²⁰-substituted or unsubstituted 6-10 membered aryl, or R²⁰-substituted or unsubstituted 5-10 membered heteroaryl.
R²⁰ is halogen, —CN, —CX²⁰₃, —CHX²⁰₂, —CH₂X²⁰, —OR^{20B}, —OC(O)R^{20B}, —OC(O)NR^{20A}R^{20B}, —C(O)—OR^{20B}, —C(O)NR^{20A}R^{20B}, —SOR^{20B}, —SO₂R^{20B}, —SO₂NR^{20A}R^{20B}, —O—P(O)(OR^{20A})₂, -(unsubstituted alkyl)-O—P(O)OR^{20A})₂,-(unsubstituted alkoxy)-O—P(O)(OR^{20A})₂, —NR^{20A}R^{20B}, NR^{20A}C(O)R^{20B}, —NR^{20A}SOR^{20B}, —NR^{20A}C(O)OR^{20B}, —NR^{20A}SO₂R^{20B}, —C(O)R^{20B}, —OCX²⁰₃, —OCH₂X²⁰, —OCHX²⁰₂, —SR^{20B}, —NHNR^{20A}R^{20B}, —NR^{20A}R^{20B}, —NHC(O)NHNR^{20A}R^{20B}, —NHC(O)NR^{20A}R^{20B}, —N(O)₂, —NR^{20A}OR^{20B}, —SF₅, —N₃, R²¹-substituted or unsubstituted saturated alkyl (e.g., C₁-C₈, C₁-C₆, C₁-C₄, or C₁-C₂), R²¹-substituted or unsubstituted alkenyl (e.g., C₂-C₈, C₂-C₆, C₂-C₄, or C₂), R²¹-substituted or unsubstituted alkynyl (e.g., C₂-C₈, C₂-C₆, C₂-C₄, or C₂), R²¹-substituted or unsubstituted heteroalkyl (e.g., 2 to 8 membered, 2 to 6 membered, 4 to 6 membered, 2 to 3 membered, or 4 to 5 membered), R²¹-substituted or unsubstituted cycloalkyl (e.g., C₃-C₈, C₃-C₆, C₄-C₆, or C₅-C₆), R²¹-substituted or unsubstituted heterocycloalkyl (e.g., 3 to 8 membered, 3 to 6 membered, 4 to 6 membered, 4 to 5 membered, or 5 to 6 membered), R²¹-substituted or unsubstituted aryl (e.g., C₆-C₁₀ or phenyl), or R²¹-substituted or unsubstituted heteroaryl (e.g., 5 to 10 membered, 5 to 9 membered, or 5 to 6 membered). X²⁰ is independently —F, —Cl, —Br, or —I.

In embodiments, R²⁰ is halogen, —CN, —CF₃, —CHF₂, —CH₂F, —OR^{20B}, —OC(O)R^{20B}, —OC(O)NR^{20A}R^{20B}, —C(O)—OR^{20B}, —C(O)NR^{20A}R^{20B}, —SOR^{20B}, —SO₂R^{20B}, —SO₂NR^{20A}R^{20B}, —O—P(O)(OR^{20A})₂, -(unsubstituted alkyl)-O—P(O)(OR^{20A})₂,-(unsubstituted alkoxy)-O—P(O)(OR^{20A})₂, —NR^{20A}R^{20B}, NR^{20A}C(O)R^{20B}, —NR^{20A}SOR^{20B}, —NR^{20A}C(O)OR^{20B}, NR^{20A}SO₂R^{20B}, —C(O)R^{20B}, —OCF₃, —OCH₂F, —OCHF₂, —SR^{20B}, —NHR^{20A}R^{20B}, —ONR^{20A}R^{20B}, —NHC(O)NHNR^{20A}R^{20B}, —NHC(O)NR^{20A}R^{20B}, —N(O)₂, —NR^{20A}OR^{20B}, —SF₅, —N₃, R²¹-substituted or unsubstituted saturated alkyl (e.g., C₁-C₈, C₁-C₆, C₁-C₄, or C₁-C₂), R²¹-substituted or unsubstituted alkenyl (e.g., C₂-C₈, C₂-C₆, C₂-C₄, or C₂), R²¹-substituted or unsubstituted alkynyl (e.g., C₂-C₈, C₂-C₆, C₂-C₄, or C₂), R²¹-substituted or unsubstituted heteroalkyl (e.g., 2 to 8 membered, 2 to 6 membered, 4 to 6 membered, 2 to 3 membered, or 4 to 5 membered), R²¹-substituted or unsubstituted cycloalkyl (e.g., C₃-C₈, C₃-C₆, C₄-C₆, or C₅-C₆), R²¹-substituted or unsubstituted heterocycloalkyl (e.g., 3 to 8 membered, 3 to 6 membered, 4 to 6 membered, 4 to 5 membered, or 5 to 6 membered), R²¹-substituted or unsubstituted aryl (e.g., C₆-C₁₀ or phenyl), or R²¹-substituted or unsubstituted heteroaryl (e.g., 5 to 10 membered, 5 to 9 membered, or 5 to 6 membered).

In embodiments, R²⁰ is halogen, —CN, —CF₃, —CHF₂, —CH₂F, —OR^{20B}, —OC(O)NR^{20A}R^{20B}, —C(O)—OR^{20B}, —C(O)NR^{20A}R^{20B}, —SOR^{20B}, —SO₂R^{20B}, —SO₂NR^{20A}R^{20B}, —O—P(O)(OR^{20A})₂, -(unsubstituted alkyl)-O—P(O)(OR^{20A})₂,-(unsubstituted alkoxy)-O—P(O)(OR^{20A})₂, NR^{20A}R^{20B}, NR^{20A}SOR^{20B}, —NR^{20A}C(O)OR^{20B}, NR^{20A}SO₂R^{20B}, —OCF₃, —OCH₂F, —OCHF₂, —SR^{20B}, —NHNR^{20A}R^{20B}, —NR^{20A}R^{20B}, —NHC(O)NHNR^{20A}R^{20B}, —NHC(O)NR^{20A}R^{20B}, —N(O)₂, —NR^{20A}OR^{20B}, —SF₅, —N₃, R²¹-substituted or unsubstituted saturated alkyl (e.g., C₁-C₈, C₁-C₆, C₁-C₄, or C₁-C₂), R²¹-substituted or unsubstituted alkenyl (e.g., C₂-C₈, C₂-C₆, C₂-C₄, or C₂), R²¹-substituted or unsubstituted alkynyl (e.g., C₂-C₈, C₂-C₆, C₂-C₄, or C₂), R²¹-substituted or unsubstituted heteroalkyl (e.g., 2 to 8 membered, 2 to 6 membered, 4 to 6 membered, 2 to 3 membered, or 4 to 5 membered), R²¹-substituted or unsubstituted cycloalkyl (e.g., C₃-C₈, C₃-C₆, C₄-C₆, or C₅-C₆), R²¹-substituted or unsubstituted heterocycloalkyl (e.g., 3 to 8 membered, 3 to 6 membered, 4 to 6 membered, 4 to 5 membered, or 5 to 6 membered), R²¹-substituted or unsubstituted aryl (e.g., C₆-C₁₀ or phenyl), or R²¹-substituted or unsubstituted heteroaryl (e.g., 5 to 10 membered, 5 to 9 membered, or 5 to 6 membered); wherein R^{20A} and R^{2B} are independently hydrogen, R²¹-substituted or unsubstituted saturated alkyl (e.g., C₁-C₁₀, C₁-C₈, C₁-C₆, C₁-C₄, or C₁-C₂), R²¹-substituted or unsubstituted alkenyl (e.g., C₂-C₁₀, C₂-C₈, C₂-C₆, C₂-C₄, or C₂), R²¹-substituted or unsubstituted alkynyl (e.g., C₂-C₁₀, C₂-C₈, C₂-C₆, C₂-C₄, or C₂), R²¹-substituted or unsubstituted heteroalkyl (e.g., 2 to 10 membered, 2 to 8 membered, 2 to 6 membered, 4 to 6 membered, 2 to 3 membered, or 4 to 5 membered), R²¹-substituted or unsubstituted cycloalkyl (e.g., C₃-C₈, C₃-C₆, C₄-C₆, or C₅-C₆), R²¹-substituted or unsubstituted heterocycloalkyl (e.g., 3 to 8 membered, 3 to 6 membered, 4 to 6 membered, 4 to 5 membered, or 5 to 6 membered), R²¹-substituted or unsubstituted aryl (e.g., C₆-C₁₀ or phenyl), or R²¹-substituted or unsubstituted heteroaryl (e.g., 5 to 10 membered, 5 to 9 membered, or 5 to 6 membered); R^{20A} and R^{20B} substituents bonded to the same nitrogen atom may optionally be joined to form a R²¹-substituted or unsubstituted heterocycloalkyl (e.g., 3 to 8 membered, 3 to 6 membered, 4 to 6 membered, 4 to 5 membered, or 5 to 6 membered) or R²¹-substituted or unsubstituted heteroaryl (e.g., 5 to 10 membered, 5 to 9 membered, or 5 to 6 membered); or 2) —OC(O)R^{20B}, —NR^{20A}C(O)R^{20B}, or —C(O)R^{20B}; wherein R^{20A} is independently hydrogen, R²¹-substituted or unsubstituted saturated alkyl (e.g., C₁-C₁₀, C₁-C₈, C₁-C₆, C₁-C₄, or C₁-C₂), R²¹-substituted or unsubstituted alkenyl (e.g., C₂-C₁₀, C₂-C₈, C₂-C₆, C₂-C₄, or C₂), R²¹-substituted or unsubstituted alkynyl (e.g., C₂-C₁₀, C₂-C₈, C₂-C₆, C₂-C₄, or C₂), R²¹-substituted or unsubstituted heteroalkyl (e.g., 2 to 10 membered, 2 to 8 membered, 2 to 6 membered, 4 to 6 membered, 2 to 3 membered, or 4 to 5 membered), R²¹-substituted or unsubstituted cycloalkyl (e.g., C₃-C₈, C₃-C₆, C₄-C₆, or C₅-C₆), R²¹-substituted or unsubstituted heterocycloalkyl (e.g., 3 to 8 membered, 3 to 6 membered, 4 to 6 membered, 4 to 5 membered, or 5 to 6 membered), R²¹-substituted or unsubstituted aryl (e.g., C₆-C₁₀ or phenyl), or R²¹-substituted or unsubstituted heteroaryl (e.g., 5 to 10 membered, 5 to 9 membered, or 5 to 6 membered); R^{20A} and R^{20B} substituents bonded to the same nitrogen atom may optionally be joined to form a R²¹-substituted or unsubstituted heterocycloalkyl (e.g., 3 to 8 membered, 3 to 6 membered, 4 to 6 membered, 4 to 5 membered, or 5 to 6 membered) or R²¹-substituted or unsubstituted heteroaryl (e.g., 5 to 10 membered, 5 to 9 membered, or 5 to 6 membered); and R^{20B} is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —COH, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R²¹-substituted or unsubstituted saturated alkyl (e.g., C₁-C₁₀, C₁-C₈, C₁-C₆, C₁-C₄, or C₁-C₂), R²¹-substituted or unsubstituted alkenyl (e.g., C₂-C₁₀, C₂-C₈, C₂-C₆, C₂-C₄, or C₂), R²¹-substituted or unsubstituted alkynyl (e.g., C₂-C₁₀, C₂-C₈, C₂-C₆, C₂-C₄, or C₂), R²¹-substituted or unsubstituted heteroalkyl (e.g., 2 to 10 membered, 2 to 8 membered, 2 to 6 membered, 4 to 6 membered, 2 to 3 membered, or 4 to 5 membered), R²¹-substituted or unsubstituted cycloalkyl (e.g., C₃-C₈, C₃-C₆, C₄-C₆, or C₅-C₆), R²¹-substituted or unsubstituted heterocycloalkyl (e.g., 3 to 8 membered, 3 to 6 membered, 4 to 6 membered, 4 to 5 membered, or 5 to 6 membered), R²¹-substituted or unsubstituted aryl (e.g., C₆-C₁₀ or phenyl), or R²¹-substituted or unsubstituted heteroaryl (e.g., 5 to 10 membered, 5 to 9 membered, or 5 to 6 membered); R^{20A} and R^{20B} substituents bonded to the same nitrogen atom may optionally be joined to form a R²¹-substituted or unsubstituted heterocycloalkyl (e.g., 3 to 8 membered, 3 to 6 membered, 4 to 6 membered, 4 to 5 membered, or 5 to 6 membered) or R²¹-substituted or unsubstituted heteroaryl (e.g., 5 to 10 membered, 5 to 9 membered, or 5 to 6 membered).

In embodiments, R⁸⁰ is halogen, —CN, —CX²⁰₃, —CHX²⁰₂, —CH₂X²⁰, —OR^{20B}, —OC(O)R^{20B}, —OC(O)NR^{20A}R^{20B}, —C(O)—OR^{20B}, —C(O)NR^{20A}R^{20B}, —SOR^{20B}, —SO₂R^{20B}, —SO₂NR^{20A}R^{2B}, —O—P(O)(OR^{20A})₂, -(unsubstituted alkyl)-O—P(O)OR^{20A})₂,-(unsubstituted alkoxy)-O—P(O)(OR^{20A})₂, —NR^{20A}R^{20B}, NR^{20A}C(O)R^{20B}, —NR^{20A}SOR^{20B}, —NR^{20A}C(O)OR^{20B}, —NR^{20A}SO₂R^{20B}, —C(O)R^{20B}, —OCX²⁰₃, —OCH₂X²⁰, —OCHX²⁰₂, —SR^{20B}, —NHNR^{20A}R^{20B}, —NR^{20A}R^{20B}, —NHC(O)NHNR^{20A}R^{20B}, —NHC(O)NR^{20A}R^{20B}, —N(O)₂, —NR^{20A}OR^{20B}, —SF₅, —N₃, R²¹-substituted or unsubstituted C₁-C₆ saturated alkyl, R²¹-substituted or unsubstituted C₂-C₆ alkenyl, R²¹-substituted or unsubstituted C₂-C₆ alkynyl, R²¹-substituted or unsubstituted 2 to 8 membered heteroalkyl, R²¹-substituted or unsubstituted cycloalkyl, R²¹-substituted or unsubstituted heterocycloalkyl, R²¹-substituted or unsubstituted aryl, or R²¹-substituted or unsubstituted heteroaryl. X²⁰ is independently —F, —Cl, —Br, or —I.
R^{20A} and R^{20B} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —COH, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R²¹-substituted or unsubstituted saturated alkyl (e.g., C₁-C₁₀, C₁-C₈, C₁-C₆, C₁-C₄, or C₁-C₂), R²¹-substituted or unsubstituted alkenyl (e.g., C₂-C₁₀, C₂-C₈, C₂-C₆, C₂-C₄, or C₂), R²¹-substituted or unsubstituted alkynyl (e.g., C₂-C₁₀, C₂-C₈, C₂-C₆, C₂-C₄, or C₂), R²¹-substituted or unsubstituted heteroalkyl (e.g., 2 to 10 membered, 2 to 8 membered, 2 to 6 membered, 4 to 6 membered, 2 to 3 membered, or 4 to 5 membered), R²¹-substituted or unsubstituted cycloalkyl (e.g., C₃-C₈, C₃-C₆, C₄-C₆, or C₅-C₆), R²¹-substituted or unsubstituted heterocycloalkyl (e.g., 3 to 8 membered, 3 to 6 membered, 4 to 6 membered, 4 to 5 membered, or 5 to 6 membered), R²¹-substituted or unsubstituted aryl (e.g., C₆-C₁₀ or phenyl), or R²¹-substituted or unsubstituted heteroaryl (e.g., 5 to 10 membered, 5 to 9 membered, or 5 to 6 membered); R^{20A} and R^{20B} substituents bonded to the same nitrogen atom may optionally be joined to form a R²¹-substituted or unsubstituted heterocycloalkyl (e.g., 3 to 8 membered, 3 to 6 membered, 4 to 6 membered, 4 to 5 membered, or 5 to 6 membered) or R²¹-substituted or unsubstituted heteroaryl (e.g., 5 to 10 membered, 5 to 9 membered, or 5 to 6 membered).

In embodiments, R^{20A} and R^{20B} are independently hydrogen, R²¹-substituted or unsubstituted saturated alkyl (e.g., C₁-C₁₀, C₁-C₈, C₁-C₆, C₁-C₄, or C₁-C₂), R²¹-substituted or unsubstituted alkenyl (e.g., C₂-C₁₀, C₂-C₈, C₂-C₆, C₂-C₄, or C₂), R²¹-substituted or unsubstituted alkynyl (e.g., C₂-C₁₀, C₂-C₈, C₂-C₆, C₂-C₄, or C₂), R²¹-substituted or unsubstituted heteroalkyl (e.g., 2 to 10 membered, 2 to 8 membered, 2 to 6 membered, 4 to 6 membered, 2 to 3 membered, or 4 to 5 membered), R²¹-substituted or unsubstituted cycloalkyl (e.g., C₃-C₈, C₃-C₆, C₄-C₆, or C₅-C₆), R²¹-substituted or unsubstituted heterocycloalkyl (e.g., 3 to 8 membered, 3 to 6 membered, 4 to 6 membered, 4 to 5 membered, or 5 to 6 membered), R²¹-substituted or unsubstituted aryl (e.g., C₆-C₁₀ or phenyl), or R²¹-substituted or unsubstituted heteroaryl (e.g., 5 to 10 membered, 5 to 9 membered, or 5 to 6 membered); R^{20A} and R^{20B} substituents bonded to the same nitrogen atom may optionally be joined to form a R²¹-substituted or unsubstituted heterocycloalkyl (e.g., 3 to 8 membered, 3 to 6 membered, 4 to 6 membered, 4 to 5 membered, or 5 to 6 membered) or R²¹-substituted or unsubstituted heteroaryl (e.g., 5 to 10 membered, 5 to 9 membered, or 5 to 6 membered).

In embodiments, R^{20A} and R^{20B} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —COH, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R²¹-substituted or unsubstituted C₁-C₁₀ saturated alkyl (e.g., C₁-C₁₀, C₁-C₈, C₁-C₆, C₁-C₄, or C₁-C₂), R²¹-substituted or unsubstituted C₂-C₁₀ alkenyl (e.g., C₂-C₁₀, C₂-C₈, C₂-C₆, C₂-C₄, or C₂), R²¹-substituted or unsubstituted C₂-C₁₀ alkynyl (e.g., C₂-C₁₀, C₂-C₈, C₂-C₆, C₂-C₄, or C₂), R²¹-substituted or unsubstituted 2 to 10 membered heteroalkyl (e.g., 2 to 10 membered, 2 to 8 membered, 2 to 6 membered, 4 to 6 membered, 2 to 3 membered, or 4 to 5 membered), R²¹-substituted or unsubstituted cycloalkyl (e.g., C₃-C₈, C₃-C₆, C₄-C₆, or C₅-C₆), R²¹-substituted or unsubstituted heterocycloalkyl (e.g., 3 to 8 membered, 3 to 6 membered, 4 to 6 membered, 4 to 5 membered, or 5 to 6 membered), R²¹-substituted or unsubstituted aryl (e.g., C₆-C₁₀ or phenyl), or R²¹-substituted or unsubstituted heteroaryl (e.g., 5 to 10 membered, 5 to 9 membered, or 5 to 6 membered); R^{20A} and R^{20B} substituents bonded to the same nitrogen atom may optionally be joined to form a R²¹-substituted or unsubstituted heterocycloalkyl (e.g., 3 to 8 membered, 3 to 6 membered, 4 to 6 membered, 4 to 5 membered, or 5 to 6 membered) or R²¹-substituted or unsubstituted heteroaryl (e.g., 5 to 10 membered, 5 to 9 membered, or 5 to 6 membered).
R²¹ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, R²²-substituted or unsubstituted saturated alkyl (e.g., C₁-C₈, C₁-C₆, C₁-C₄, or C₁-C₂), R²²-substituted or unsubstituted alkenyl (e.g., C₂-C₈, C₂-C₆, C₂-C₄, or C₂), R²²-substituted or unsubstituted alkynyl (e.g., C₂-C₈, C₂-C₆, C₂-C₄, or C₂), R²²-substituted or unsubstituted heteroalkyl (e.g., 2 to 12 membered, 2 to 6 membered, 4 to 6 membered, 2 to 3 membered, or 4 to 5 membered), R²²-substituted or unsubstituted cycloalkyl (e.g., C₃-C₈, C₃-C₆, C₄-C₆, or C₅-C₆), R²²-substituted or unsubstituted heterocycloalkyl (e.g., 3 to 15 membered, 3 to 6 membered, 4 to 6 membered, 4 to 5 membered, or 5 to 6 membered), R²²-substituted or unsubstituted aryl (e.g., C₆-C₁₀ or phenyl), or R²²-substituted or unsubstituted heteroaryl (e.g., 5 to 10 membered, 5 to 9 membered, or 5 to 6 membered).

In embodiments, R²¹ is independently oxo, halogen, —CF₃, —CHF₂, —CH₂F, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSO₂H, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, —OCH₂F, —SF₅, —N₃, —O—P(O)(OH)₂, R²²-substituted or unsubstituted saturated alkyl (e.g., C₁-C₈, C₁-C₆, C₁-C₄, or C₁-C₂), R²²-substituted or unsubstituted alkenyl (e.g., C₂-C₈, C₂-C₆, C₂-C₄, or C₂), R²²-substituted or unsubstituted alkynyl (e.g., C₂-C₈, C₂-C₆, C₂-C₄, or C₂), R²²-substituted or unsubstituted heteroalkyl (e.g., 2 to 8 membered, 2 to 6 membered, 4 to 6 membered, 2 to 3 membered, or 4 to 5 membered), R²²-substituted or unsubstituted cycloalkyl (e.g., C₃-C₈, C₃-C₆, C₄-C₆, or C₅-C₆), R²²-substituted or unsubstituted heterocycloalkyl (e.g., 3 to 8 membered, 3 to 6 membered, 4 to 6 membered, 4 to 5 membered, or 5 to 6 membered), R²²-substituted or unsubstituted aryl (e.g., C₆-C₁₀ or phenyl), or R²²-substituted or unsubstituted heteroaryl (e.g., 5 to 10 membered, 5 to 9 membered, or 5 to 6 membered).

In embodiments, R²¹ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, R²²-substituted or unsubstituted C₁-C₄ saturated alkyl, R²²-substituted or unsubstituted C₂-C₄ alkenyl, R²²-substituted or unsubstituted C₂-C₄ alkynyl, R²²-substituted or unsubstituted 2 to 12 membered heteroalkyl, R²²-substituted or unsubstituted C₃-C₆ cycloalkyl, R²²-substituted or unsubstituted 3 to 15 membered heterocycloalkyl, R²²-substituted or unsubstituted phenyl, or R²²-substituted or unsubstituted 5 to 6 membered heteroaryl.
R²² is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, R²² is independently oxo, halogen, —CF₃, —CHF₂, —CH₂F, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSO₂H, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, —OCH₂F, —SF₅, —N₃, —O—P(O)(OH)₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, R³ is independently halogen, —OR^{3D}, —CN, —CX³₃, —CHX³₂, —CH₂X³, —C(O)NR^{3A}R^{3B}, —C(O)—OR^{3C}, —O—P(O)(OH)₂, —NR^{3A}R^{3B}, —C(O)R^{3C}, —NR^{3A}C(O)R^{3C}, —NR^{3A}SO₂R^{3D}, —NR^{3C}SO₂(unsubstituted C₁-C₆ alkyl)-NR^{3A}R^{3B}, —NR^{3C}C(O)(unsubstituted C₁-C₆alkyl)-NR^{3A}R^{3B}, (unsubstituted C₂-C₆alkyl)-NR^{3E}-(unsubstituted C₂-C₆ alkyl), R³¹-substituted or unsubstituted alkyl, R³¹-substituted or unsubstituted heteroalkyl, or R³¹-substituted or unsubstituted cycloalkyl.

In embodiments, R³ is independently halogen, —OR^{3D}, —CN, —CF₃, —CHF₂, —CH₂F, —C(O)NR^{3A}R^{3B}, —C(O)—OR^{3C}, —O—P(O)(OH)₂, —NR^{3A}R^{3B}, —C(O)R^{3C}, —NR^{3A}C(O)R^{3C}, —NR^{3A}SO₂R^{3D}, —NR^{3C}SO₂(unsubstituted C₁-C₆ alkyl)-NR^{3A}R^{3B}, NR^{3C}C(O)(unsubstituted C₁-C₆alkyl)-NR^{3A}R^{3B}, (unsubstituted C₂-C₆alkyl)-NR^{3E}-(unsubstituted C₂-C₆ alkyl), R³¹-substituted or unsubstituted alkyl, R³¹-substituted or unsubstituted heteroalkyl, or R³¹-substituted or unsubstituted cycloalkyl.

In embodiments, R^{3A}, R^{3B}, R^{3C}, and R^{3D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R³¹-substituted or unsubstituted alkyl, R³¹-substituted or unsubstituted heteroalkyl, R³¹-substituted or unsubstituted cycloalkyl, R³¹-substituted or unsubstituted heterocycloalkyl, R³¹-substituted or unsubstituted aryl, or R³¹-substituted or unsubstituted heteroaryl; R^{3A} and R^{3B} substituents bonded to the same nitrogen atom may optionally be joined to form a R³¹-substituted or unsubstituted heterocycloalkyl or R³¹-substituted or unsubstituted heteroaryl; R^{3F} and R^{3G} substituents bonded to the same nitrogen atom may optionally be joined to form a R³¹-substituted or unsubstituted heterocycloalkyl (e.g., 3 to 8 membered, 3 to 6 membered, 4 to 6 membered, 4 to 5 membered, or 5 to 6 membered) or R³¹-substituted or unsubstituted heteroaryl (e.g., 5 to 10 membered, 5 to 9 membered, or 5 to 6 membered).

In embodiments, R^{3A}, R^{3B}, R^{3C}, and R^{3D} are independently hydrogen, R³¹-substituted or unsubstituted alkyl, R³¹-substituted or unsubstituted heteroalkyl, R³¹-substituted or unsubstituted cycloalkyl, R³¹-substituted or unsubstituted heterocycloalkyl, R³¹-substituted or unsubstituted aryl, or R³¹-substituted or unsubstituted heteroaryl; R^{3A} and R^{3B} substituents bonded to the same nitrogen atom may optionally be joined to form a R³¹-substituted or unsubstituted heterocycloalkyl or R³¹-substituted or unsubstituted heteroaryl; R^{3F} and R^{3G} substituents bonded to the same nitrogen atom may optionally be joined to form a R³¹-substituted or unsubstituted heterocycloalkyl (e.g., 3 to 8 membered, 3 to 6 membered, 4 to 6 membered, 4 to 5 membered, or 5 to 6 membered) or R³¹-substituted or unsubstituted heteroaryl (e.g., 5 to 10 membered, 5 to 9 membered, or 5 to 6 membered).
R^{3F} and R^{3G} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R³¹-substituted or unsubstituted alkyl (e.g., C₁-C₈, C₁-C₆, C₁-C₄, or C₁-C₂), R³¹-substituted or unsubstituted heteroalkyl (e.g., 2 to 8 membered, 2 to 6 membered, 4 to 6 membered, 2 to 3 membered, or 4 to 5 membered), R³¹-substituted or unsubstituted cycloalkyl (e.g., C₃-C₈, C₃-C₆, C₄-C₆, or C₅-C₆), R³¹-substituted or unsubstituted heterocycloalkyl (e.g., 3 to 8 membered, 3 to 6 membered, 4 to 6 membered, 4 to 5 membered, or 5 to 6 membered), R³¹-substituted or unsubstituted aryl (e.g., C₆-C₁₀ or phenyl), or R³¹-substituted or unsubstituted heteroaryl (e.g., 5 to 10 membered, 5 to 9 membered, or 5 to 6 membered); R^{3A} and R^{3B} substituents bonded to the same nitrogen atom may optionally be joined to form a R³¹-substituted or unsubstituted heterocycloalkyl (e.g., 3 to 8 membered, 3 to 6 membered, 4 to 6 membered, 4 to 5 membered, or 5 to 6 membered) or R³¹-substituted or unsubstituted heteroaryl (e.g., 5 to 10 membered, 5 to 9 membered, or 5 to 6 membered); R^{3F} and R^{3G} substituents bonded to the same nitrogen atom may optionally be joined to form a R³¹-substituted or unsubstituted heterocycloalkyl (e.g., 3 to 8 membered, 3 to 6 membered, 4 to 6 membered, 4 to 5 membered, or 5 to 6 membered) or R³-substituted or unsubstituted heteroaryl (e.g., 5 to 10 membered, 5 to 9 membered, or 5 to 6 membered).

In embodiments, R^{3F} and R^{3G} are independently hydrogen, R³¹-substituted or unsubstituted alkyl (e.g., C₁-C₈, C₁-C₆, C₁-C₄, or C₁-C₂), R³¹-substituted or unsubstituted heteroalkyl (e.g., 2 to 8 membered, 2 to 6 membered, 4 to 6 membered, 2 to 3 membered, or 4 to 5 membered), R³¹-substituted or unsubstituted cycloalkyl (e.g., C₃-C₈, C₃-C₆, C₄-C₆, or C₅-C₆), R³¹-substituted or unsubstituted heterocycloalkyl (e.g., 3 to 8 membered, 3 to 6 membered, 4 to 6 membered, 4 to 5 membered, or 5 to 6 membered), R³¹-substituted or unsubstituted aryl (e.g., C₆-C₁₀ or phenyl), or R³-substituted or unsubstituted heteroaryl (e.g., 5 to 10 membered, 5 to 9 membered, or 5 to 6 membered); R^{3F} and R^{3G} substituents bonded to the same nitrogen atom may optionally be joined to form a R³-substituted or unsubstituted heterocycloalkyl (e.g., 3 to 8 membered, 3 to 6 membered, 4 to 6 membered, 4 to 5 membered, or 5 to 6 membered) or R³-substituted or unsubstituted heteroaryl (e.g., 5 to 10 membered, 5 to 9 membered, or 5 to 6 membered).

In embodiments, R^{3A}, R^{3B}, R^{3C}, R^{3D}, R^{3F}, and R^{3G} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R³¹-substituted or unsubstituted alkyl (e.g., C₁-C₈, C₁-C₆, C₁-C₄, or C₁-C₂), R³¹-substituted or unsubstituted heteroalkyl (e.g., 2 to 8 membered, 2 to 6 membered, 4 to 6 membered, 2 to 3 membered, or 4 to 5 membered), R³¹-substituted or unsubstituted cycloalkyl (e.g., C₃-C₈, C₃-C₆, C₄-C₆, or C₅-C₆), R³¹-substituted or unsubstituted heterocycloalkyl (e.g., 3 to 8 membered, 3 to 6 membered, 4 to 6 membered, 4 to 5 membered, or 5 to 6 membered), R³¹-substituted or unsubstituted aryl (e.g., C₆-C₁₀ or phenyl), or R³¹-substituted or unsubstituted heteroaryl (e.g., 5 to 10 membered, 5 to 9 membered, or 5 to 6 membered); R^{3A} and R^{3B} substituents bonded to the same nitrogen atom may optionally be joined to form a R³¹-substituted or unsubstituted heterocycloalkyl (e.g., 3 to 8 membered, 3 to 6 membered, 4 to 6 membered, 4 to 5 membered, or 5 to 6 membered) or R³¹-substituted or unsubstituted heteroaryl (e.g., 5 to 10 membered, 5 to 9 membered, or 5 to 6 membered); R^{3F} and R^{3G} substituents bonded to the same nitrogen atom may optionally be joined to form a R³¹-substituted or unsubstituted heterocycloalkyl (e.g., 3 to 8 membered, 3 to 6 membered, 4 to 6 membered, 4 to 5 membered, or 5 to 6 membered) or R³-substituted or unsubstituted heteroaryl (e.g., 5 to 10 membered, 5 to 9 membered, or 5 to 6 membered).

In embodiments, R^{3A}, R^{3B}, R^{3C}, R^{3D}, R^{3F}, and R^{3G} are independently hydrogen, R³¹-substituted or unsubstituted alkyl (e.g., C₁-C₈, C₁-C₆, C₁-C₄, or C₁-C₂), R³¹-substituted or unsubstituted heteroalkyl (e.g., 2 to 8 membered, 2 to 6 membered, 4 to 6 membered, 2 to 3 membered, or 4 to 5 membered), R³¹-substituted or unsubstituted cycloalkyl (e.g., C₃-C₈, C₃-C₆, C₄-C₆, or C₅-C₆), R³¹-substituted or unsubstituted heterocycloalkyl (e.g., 3 to 8 membered, 3 to 6 membered, 4 to 6 membered, 4 to 5 membered, or 5 to 6 membered), R³¹-substituted or unsubstituted aryl (e.g., C₆-C₁₀ or phenyl), or R³¹-substituted or unsubstituted heteroaryl (e.g., 5 to 10 membered, 5 to 9 membered, or 5 to 6 membered); R^{3A} and R^{3B} substituents bonded to the same nitrogen atom may optionally be joined to form a R³¹-substituted or unsubstituted heterocycloalkyl (e.g., 3 to 8 membered, 3 to 6 membered, 4 to 6 membered, 4 to 5 membered, or 5 to 6 membered) or R³¹-substituted or unsubstituted heteroaryl (e.g., 5 to 10 membered, 5 to 9 membered, or 5 to 6 membered); R^{3F} and R^{3G} substituents bonded to the same nitrogen atom may optionally be joined to form a R³¹-substituted or unsubstituted heterocycloalkyl (e.g., 3 to 8 membered, 3 to 6 membered, 4 to 6 membered, 4 to 5 membered, or 5 to 6 membered) or R³-substituted or unsubstituted heteroaryl (e.g., 5 to 10 membered, 5 to 9 membered, or 5 to 6 membered).
R^{3E} is independently hydrogen, —C(O)R^{3F}, —C(O)—OR^{3F}, —SOR^{3F}, —SO₂R^{3F}, —C(O)NR^{3F}R^{3G}, —SO₂NR^{3F}R^{3G}; —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R³¹-substituted or unsubstituted alkyl (e.g., C₁-C₈, C₁-C₆, C₁-C₄, or C₁-C₂), R³¹-substituted or unsubstituted heteroalkyl (e.g., 2 to 8 membered, 2 to 6 membered, 4 to 6 membered, 2 to 3 membered, or 4 to 5 membered), R³¹-substituted or unsubstituted cycloalkyl (e.g., C₃-C₈, C₃-C₆, C₄-C₆, or C₅-C₆), R³¹-substituted or unsubstituted heterocycloalkyl (e.g., 3 to 8 membered, 3 to 6 membered, 4 to 6 membered, 4 to 5 membered, or 5 to 6 membered), R³¹-substituted or unsubstituted aryl (e.g., C₆-C₁₀ or phenyl), or R³¹-substituted or unsubstituted heteroaryl (e.g., 5 to 10 membered, 5 to 9 membered, or 5 to 6 membered); R^{3A} and R^{3B} substituents bonded to the same nitrogen atom may optionally be joined to form a R³¹-substituted or unsubstituted heterocycloalkyl (e.g., 3 to 8 membered, 3 to 6 membered, 4 to 6 membered, 4 to 5 membered, or 5 to 6 membered) or R³¹-substituted or unsubstituted heteroaryl (e.g., 5 to 10 membered, 5 to 9 membered, or 5 to 6 membered); R^{3F} and R^{3G} substituents bonded to the same nitrogen atom may optionally be joined to form a R³¹-substituted or unsubstituted heterocycloalkyl (e.g., 3 to 8 membered, 3 to 6 membered, 4 to 6 membered, 4 to 5 membered, or 5 to 6 membered) or R³-substituted or unsubstituted heteroaryl (e.g., 5 to 10 membered, 5 to 9 membered, or 5 to 6 membered).

In embodiments, R^{3E} is independently hydrogen, —C(O)R^{3F}, —C(O)—OR^{3F}, —SOR^{3F}, —SO₂R^{3F}, —C(O)NR^{3F}R^{3G}, —SO₂NR^{3F}R^{3G}, R³¹-substituted or unsubstituted alkyl (e.g., C₁-C₈, C₁-C₆, C₁-C₄, or C₁-C₂), R³¹-substituted or unsubstituted heteroalkyl (e.g., 2 to 8 membered, 2 to 6 membered, 4 to 6 membered, 2 to 3 membered, or 4 to 5 membered), R³¹-substituted or unsubstituted cycloalkyl (e.g., C₃-C₈, C₃-C₆, C₄-C₆, or C₅-C₆), R³¹-substituted or unsubstituted heterocycloalkyl (e.g., 3 to 8 membered, 3 to 6 membered, 4 to 6 membered, 4 to 5 membered, or 5 to 6 membered), R³¹-substituted or unsubstituted aryl (e.g., C₆-C₁₀ or phenyl), or R³¹-substituted or unsubstituted heteroaryl (e.g., 5 to 10 membered, 5 to 9 membered, or 5 to 6 membered).

In embodiments, R″ is independently 1) hydrogen, —C(O)—OR^{3F}, —SOR^{3F}, —SO₂R^{3F}, —C(O)NR^{3F}R^{3G}, —SO₂NR^{3F}R^{3G}, R³¹-substituted or unsubstituted alkyl (e.g., C₁-C₈, C₁-C₆, C₁-C₄, or C₁-C₂), R³¹-substituted or unsubstituted heteroalkyl (e.g., 2 to 8 membered, 2 to 6 membered, 4 to 6 membered, 2 to 3 membered, or 4 to 5 membered), R³¹-substituted or unsubstituted cycloalkyl (e.g., C₃-C₈, C₃-C₆, C₄-C₆, or C₅-C₆), R³¹-substituted or unsubstituted heterocycloalkyl (e.g., 3 to 8 membered, 3 to 6 membered, 4 to 6 membered, 4 to 5 membered, or 5 to 6 membered), R³¹-substituted or unsubstituted aryl (e.g., C₆-C₁₀ or phenyl), or R³-substituted or unsubstituted heteroaryl (e.g., 5 to 10 membered, 5 to 9 membered, or 5 to 6 membered); wherein R^{3F} and R^{3G} are independently hydrogen, R³¹-substituted or unsubstituted alkyl (e.g., C₁-C₈, C₁-C₆, C₁-C₄, or C₁-C₂), R³¹-substituted or unsubstituted heteroalkyl (e.g., 2 to 8 membered, 2 to 6 membered, 4 to 6 membered, 2 to 3 membered, or 4 to 5 membered), R³¹-substituted or unsubstituted cycloalkyl (e.g., C₃-C₈, C₃-C₆, C₄-C₆, or C₅-C₆), R³¹-substituted or unsubstituted heterocycloalkyl (e.g., 3 to 8 membered, 3 to 6 membered, 4 to 6 membered, 4 to 5 membered, or 5 to 6 membered), R³¹-substituted or unsubstituted aryl (e.g., C₆-C₁₀ or phenyl), or R³-substituted or unsubstituted heteroaryl (e.g., 5 to 10 membered, 5 to 9 membered, or 5 to 6 membered); R^{3F} and R^{3G} substituents bonded to the same nitrogen atom may optionally be joined to form a R³¹-substituted or unsubstituted heterocycloalkyl (e.g., 3 to 8 membered, 3 to 6 membered, 4 to 6 membered, 4 to 5 membered, or 5 to 6 membered) or R³-substituted or unsubstituted heteroaryl (e.g., 5 to 10 membered, 5 to 9 membered, or 5 to 6 membered); or 2) —C(O)R^{3F}; wherein R^{3F} is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R³¹-substituted or unsubstituted alkyl (e.g., C₁-C₈, C₁-C₆, C₁-C₄, or C₁-C₂), R³¹-substituted or unsubstituted heteroalkyl (e.g., 2 to 8 membered, 2 to 6 membered, 4 to 6 membered, 2 to 3 membered, or 4 to 5 membered), R³¹-substituted or unsubstituted cycloalkyl (e.g., C₃-C₈, C₃-C₆, C₄-C₆, or C₅-C₆), R³¹-substituted or unsubstituted heterocycloalkyl (e.g., 3 to 8 membered, 3 to 6 membered, 4 to 6 membered, 4 to 5 membered, or 5 to 6 membered), R³¹-substituted or unsubstituted aryl (e.g., C₆-C₁₀ or phenyl), or R³-substituted or unsubstituted heteroaryl (e.g., 5 to 10 membered, 5 to 9 membered, or 5 to 6 membered).
R³¹ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, R³²-substituted or unsubstituted saturated alkyl (e.g., C₁-C₈, C₁-C₆, C₁-C₄, or C₁-C₂), R³²-substituted or unsubstituted alkenyl (e.g., C₂-C₈, C₂-C₆, C₂-C₄, or C₂), R³²-substituted or unsubstituted alkynyl (e.g., C₂-C₈, C₂-C₆, C₂-C₄, or C₂), R³²-substituted or unsubstituted heteroalkyl (e.g., 2 to 12 membered, 2 to 6 membered, 4 to 6 membered, 2 to 3 membered, or 4 to 5 membered), R³²-substituted or unsubstituted cycloalkyl (e.g., C₃-C₈, C₃-C₆, C₄-C₆, or C₅-C₆), R³²-substituted or unsubstituted heterocycloalkyl (e.g., 3 to 15 membered, 3 to 6 membered, 4 to 6 membered, 4 to 5 membered, or 5 to 6 membered), R³²-substituted or unsubstituted aryl (e.g., C₆-C₁₀ or phenyl), or R³²-substituted or unsubstituted heteroaryl (e.g., 5 to 10 membered, 5 to 9 membered, or 5 to 6 membered).

In embodiments, R³¹ is independently oxo, halogen, —CF₃, —CHF₂, —CH₂F, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSO₂H, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, —OCH₂F, —SF₅, —N₃, —O—P(O)(OH)₂, R³²-substituted or unsubstituted saturated alkyl (e.g., C₁-C₈, C₁-C₆, C₁-C₄, or C₁-C₂), R³²-substituted or unsubstituted alkenyl (e.g., C₂-C₈, C₂-C₆, C₂-C₄, or C₂), R³²-substituted or unsubstituted alkynyl (e.g., C₂-C₈, C₂-C₆, C₂-C₄, or C₂), R³²-substituted or unsubstituted heteroalkyl (e.g., 2 to 8 membered, 2 to 6 membered, 4 to 6 membered, 2 to 3 membered, or 4 to 5 membered), R³²-substituted or unsubstituted cycloalkyl (e.g., C₃-C₈, C₃-C₆, C₄-C₆, or C₅-C₆), R³²-substituted or unsubstituted heterocycloalkyl (e.g., 3 to 8 membered, 3 to 6 membered, 4 to 6 membered, 4 to 5 membered, or 5 to 6 membered), R³²-substituted or unsubstituted aryl (e.g., C₆-C₁₀ or phenyl), or R³²-substituted or unsubstituted heteroaryl (e.g., 5 to 10 membered, 5 to 9 membered, or 5 to 6 membered).

In embodiments, R³¹ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, R³²-substituted or unsubstituted C₁-C₄ saturated alkyl, R³²-substituted or unsubstituted C₂-C₄ alkenyl, R³²-substituted or unsubstituted C₂-C₄ alkynyl, R³²-substituted or unsubstituted 2 to 12 membered heteroalkyl, R³²-substituted or unsubstituted C₃-C₆ cycloalkyl, R³²-substituted or unsubstituted 3 to 15 membered heterocycloalkyl, R³²-substituted or unsubstituted phenyl, or R³²-substituted or unsubstituted 5 to 6 membered heteroaryl.
R³² is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, R³³-substituted or unsubstituted saturated alkyl (e.g., C₁-C₈, C₁-C₆, C₁-C₄, or C₁-C₂), R³³-substituted or unsubstituted alkenyl (e.g., C₂-C₈, C₂-C₆, C₂-C₄, or C₂), R³³-substituted or unsubstituted alkynyl (e.g., C₂-C₈, C₂-C₆, C₂-C₄, or C₂), R³³-substituted or unsubstituted heteroalkyl (e.g., 2 to 12 membered, 2 to 6 membered, 4 to 6 membered, 2 to 3 membered, or 4 to 5 membered), R³³-substituted or unsubstituted cycloalkyl (e.g., C₃-C₈, C₃-C₆, C₄-C₆, or C₅-C₆), R³³-substituted or unsubstituted heterocycloalkyl (e.g., 3 to 15 membered, 3 to 6 membered, 4 to 6 membered, 4 to 5 membered, or 5 to 6 membered), R³³-substituted or unsubstituted aryl (e.g., C₆-C₁₀ or phenyl), or R³³-substituted or unsubstituted heteroaryl (e.g., 5 to 10 membered, 5 to 9 membered, or 5 to 6 membered).

In embodiments, R³² is independently oxo, halogen, —CF₃, —CHF₂, —CH₂F, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSO₂H, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, —OCH₂F, —SF₅, —N₃, —O—P(O)(OH)₂, R³³-substituted or unsubstituted saturated alkyl (e.g., C₁-C₈, C₁-C₆, C₁-C₄, or C₁-C₂), R³³-substituted or unsubstituted alkenyl (e.g., C₂-C₈, C₂-C₆, C₂-C₄, or C₂), R³³-substituted or unsubstituted alkynyl (e.g., C₂-C₈, C₂-C₆, C₂-C₄, or C₂), R³³-substituted or unsubstituted heteroalkyl (e.g., 2 to 8 membered, 2 to 6 membered, 4 to 6 membered, 2 to 3 membered, or 4 to 5 membered), R³³-substituted or unsubstituted cycloalkyl (e.g., C₃-C₈, C₃-C₆, C₄-C₆, or C₅-C₆), R³³-substituted or unsubstituted heterocycloalkyl (e.g., 3 to 8 membered, 3 to 6 membered, 4 to 6 membered, 4 to 5 membered, or 5 to 6 membered), R³³-substituted or unsubstituted aryl (e.g., C₆-C₁₀ or phenyl), or R³³-substituted or unsubstituted heteroaryl (e.g., 5 to 10 membered, 5 to 9 membered, or 5 to 6 membered).

In embodiments, R³² is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, R³³-substituted or unsubstituted C₁-C₄ saturated alkyl, R³³-substituted or unsubstituted C₂-C₄ alkenyl, R³³-substituted or unsubstituted C₂-C₄ alkynyl, R³³-substituted or unsubstituted 2 to 12 membered heteroalkyl, R³³-substituted or unsubstituted C₃-C₆ cycloalkyl, R³³-substituted or unsubstituted 3 to 15 membered heterocycloalkyl, R³³-substituted or unsubstituted phenyl, or R³³-substituted or unsubstituted 5 to 6 membered heteroaryl.
R³³ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, R³³ is independently oxo, halogen, —CF₃, —CHF₂, —CH₂F, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSO₂H, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, —OCH₂F, —SF₅, —N₃, —O—P(O)(OH)₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, R⁴ is independently halogen, —OR^{4D}, —CN, —CX⁴₃, —CHX⁴₂, —CH₂X⁴, —C(O)NR^{4A}R^{4B}, —C(O)—OR^{4C}, —O—P(O)(OH)₂, —NR^{4A}R^{4B}, —C(O)R^{4C}, —NR^{4A}C(O)R^{4C}, —N^{4A}SO₂R^{4D}, —NR^{4C}SO₂(unsubstituted C₁-C₆ alkyl)-NR^{4A}R^{4B}, —NR^{4C}C(O)(unsubstituted C₁-C₆alkyl)-NR^{4A}R^{4B}, (unsubstituted C₂-C₆alkyl)-NR^{4E}-(unsubstituted C₂-C₆ alkyl), R⁴¹-substituted or unsubstituted alkyl (e.g., C₁-C₈, C₁-C₆, C₁-C₄, or C₁-C₂), R^{4′}-substituted or unsubstituted heteroalkyl (e.g., 2 to 8 membered, 2 to 6 membered, 4 to 6 membered, 2 to 3 membered, or 4 to 5 membered), or R⁴¹-substituted or unsubstituted cycloalkyl (e.g., C₃-C₈, C₃-C₆, C₄-C₆, or C₅-C₆).

In embodiments, R⁴ is independently halogen, —OR^{4D}, —CN, —CF₃, —CHF₂, —CH₂F, —C(O)NR^{4A}R^{B}, —C(O)—OR^{c}, —O—P(O)(OH)₂, —NR^{4A}R^{4B}, —C(O)R^{4C}, —NR^{4A}C(O)R^{4C}, —N^{4A}SO₂R^{4D}, —NR^{4C}SO₂(unsubstituted C₁-C₆ alkyl)-NR^{4A}R^{4B}, —N^{4C}C(O)(unsubstituted C₁-C₆ alkyl)-NR^{4A}R^{4B}, (unsubstituted C₂-C₆ alkyl)-N^{4E}-unsubstituted C₂-C₆alkyl), R⁴¹-substituted or unsubstituted alkyl (e.g., C₁-C₈, C₁-C₆, C₁-C₄, or C₁-C₂), R⁴¹-substituted or unsubstituted heteroalkyl (e.g., 2 to 8 membered, 2 to 6 membered, 4 to 6 membered, 2 to 3 membered, or 4 to 5 membered), or R⁴-substituted or unsubstituted cycloalkyl (e.g., C₃-C₈, C₃-C₆, C₄-C₆, or C₅-C₆).

In embodiments, R^{4A}, R^{4B}, R^{4C}, and R^{4D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R⁴¹-substituted or unsubstituted alkyl (e.g., C₁-C₈, C₁-C₆, C₁-C₄, or C₁-C₂), R⁴¹-substituted or unsubstituted heteroalkyl (e.g., 2 to 8 membered, 2 to 6 membered, 4 to 6 membered, 2 to 3 membered, or 4 to 5 membered), R⁴¹-substituted or unsubstituted cycloalkyl (e.g., C₃-C₈, C₃-C₆, C₄-C₆, or C₅-C₆), R⁴¹-substituted or unsubstituted heterocycloalkyl (e.g., 3 to 8 membered, 3 to 6 membered, 4 to 6 membered, 4 to 5 membered, or 5 to 6 membered), R⁴¹-substituted or unsubstituted aryl (e.g., C₆-C₁₀ or phenyl), or R⁴-substituted or unsubstituted heteroaryl (e.g., 5 to 10 membered, 5 to 9 membered, or 5 to 6 membered); R^{4A} and R^{4B} substituents bonded to the same nitrogen atom may optionally be joined to form a R⁴¹-substituted or unsubstituted heterocycloalkyl (e.g., 3 to 8 membered, 3 to 6 membered, 4 to 6 membered, 4 to 5 membered, or 5 to 6 membered) or R⁴-substituted or unsubstituted heteroaryl (e.g., 5 to 10 membered, 5 to 9 membered, or 5 to 6 membered); R^{4F} and R^{4G} substituents bonded to the same nitrogen atom may optionally be joined to form a R⁴¹-substituted or unsubstituted heterocycloalkyl (e.g., 3 to 8 membered, 3 to 6 membered, 4 to 6 membered, 4 to 5 membered, or 5 to 6 membered) or R⁴¹ substituted or unsubstituted heteroaryl (e.g., 5 to 10 membered, 5 to 9 membered, or 5 to 6 membered).

In embodiments, R^{4A}, R^{4B}, R^{4C}, and R^{4D} are independently hydrogen, R⁴¹-substituted or unsubstituted alkyl (e.g., C₁-C₈, C₁-C₆, C₁-C₄, or C₁-C₂), R⁴¹-substituted or unsubstituted heteroalkyl (e.g., 2 to 8 membered, 2 to 6 membered, 4 to 6 membered, 2 to 3 membered, or 4 to 5 membered), R⁴¹-substituted or unsubstituted cycloalkyl (e.g., C₃-C₈, C₃-C₆, C₄-C₆, or C₅-C₆), R⁴¹-substituted or unsubstituted heterocycloalkyl (e.g., 3 to 8 membered, 3 to 6 membered, 4 to 6 membered, 4 to 5 membered, or 5 to 6 membered), R⁴¹-substituted or unsubstituted aryl (e.g., C₆-C₁₀ or phenyl), or R⁴-substituted or unsubstituted heteroaryl (e.g., 5 to 10 membered, 5 to 9 membered, or 5 to 6 membered); R^{4A} and R^{4B} substituents bonded to the same nitrogen atom may optionally be joined to form a R⁴¹-substituted or unsubstituted heterocycloalkyl (e.g., 3 to 8 membered, 3 to 6 membered, 4 to 6 membered, 4 to 5 membered, or 5 to 6 membered) or R⁴-substituted or unsubstituted heteroaryl (e.g., 5 to 10 membered, 5 to 9 membered, or 5 to 6 membered); R^{4F} and R^{4G} substituents bonded to the same nitrogen atom may optionally be joined to form a R⁴¹-substituted or unsubstituted heterocycloalkyl (e.g., 3 to 8 membered, 3 to 6 membered, 4 to 6 membered, 4 to 5 membered, or 5 to 6 membered) or R⁴¹-substituted or unsubstituted heteroaryl (e.g., 5 to 10 membered, 5 to 9 membered, or 5 to 6 membered).
R^{4F} and R^{4G} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R⁴¹-substituted or unsubstituted alkyl (e.g., C₁-C₈, C₁-C₆, C₁-C₄, or C₁-C₂), R⁴¹-substituted or unsubstituted heteroalkyl (e.g., 2 to 8 membered, 2 to 6 membered, 4 to 6 membered, 2 to 3 membered, or 4 to 5 membered), R⁴¹-substituted or unsubstituted cycloalkyl (e.g., C₃-C₈, C₃-C₆, C₄-C₆, or C₅-C₆), R⁴¹-substituted or unsubstituted heterocycloalkyl (e.g., 3 to 8 membered, 3 to 6 membered, 4 to 6 membered, 4 to 5 membered, or 5 to 6 membered), R⁴¹-substituted or unsubstituted aryl (e.g., C₆-C₁₀ or phenyl), or R⁴¹-substituted or unsubstituted heteroaryl (e.g., 5 to 10 membered, 5 to 9 membered, or 5 to 6 membered); R^{4A} and R^{4B} substituents bonded to the same nitrogen atom may optionally be joined to form a R⁴¹-substituted or unsubstituted heterocycloalkyl (e.g., 3 to 8 membered, 3 to 6 membered, 4 to 6 membered, 4 to 5 membered, or 5 to 6 membered) or R⁴¹-substituted or unsubstituted heteroaryl (e.g., 5 to 10 membered, 5 to 9 membered, or 5 to 6 membered); R^{4F} and R^{4G} substituents bonded to the same nitrogen atom may optionally be joined to form a R⁴¹-substituted or unsubstituted heterocycloalkyl (e.g., 3 to 8 membered, 3 to 6 membered, 4 to 6 membered, 4 to 5 membered, or 5 to 6 membered) or R⁴-substituted or unsubstituted heteroaryl (e.g., 5 to 10 membered, 5 to 9 membered, or 5 to 6 membered).

In embodiments, R^{4F} and R^{4G} are independently hydrogen, R⁴¹-substituted or unsubstituted alkyl (e.g., C₁-C₈, C₁-C₆, C₁-C₄, or C₁-C₂), R⁴¹-substituted or unsubstituted heteroalkyl (e.g., 2 to 8 membered, 2 to 6 membered, 4 to 6 membered, 2 to 3 membered, or 4 to 5 membered), R⁴¹-substituted or unsubstituted cycloalkyl (e.g., C₃-C₈, C₃-C₆, C₄-C₆, or C₅-C₆), R⁴¹-substituted or unsubstituted heterocycloalkyl (e.g., 3 to 8 membered, 3 to 6 membered, 4 to 6 membered, 4 to 5 membered, or 5 to 6 membered), R⁴¹-substituted or unsubstituted aryl (e.g., C₆-C₁₀ or phenyl), or R⁴¹-substituted or unsubstituted heteroaryl (e.g., 5 to 10 membered, 5 to 9 membered, or 5 to 6 membered); R^{4A} and R^{4B} substituents bonded to the same nitrogen atom may optionally be joined to form a R⁴¹-substituted or unsubstituted heterocycloalkyl (e.g., 3 to 8 membered, 3 to 6 membered, 4 to 6 membered, 4 to 5 membered, or 5 to 6 membered) or R⁴-substituted or unsubstituted heteroaryl (e.g., 5 to 10 membered, 5 to 9 membered, or 5 to 6 membered); R^{4F} and R^{4G} substituents bonded to the same nitrogen atom may optionally be joined to form a R⁴¹-substituted or unsubstituted heterocycloalkyl (e.g., 3 to 8 membered, 3 to 6 membered, 4 to 6 membered, 4 to 5 membered, or 5 to 6 membered) or R⁴¹-substituted or unsubstituted heteroaryl (e.g., 5 to 10 membered, 5 to 9 membered, or 5 to 6 membered).

In embodiments, R^{4A}, R^{4B}, R^{4C}, R^{4D}, R^{4F}, and R^{4G} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R⁴¹-substituted or unsubstituted alkyl, R⁴¹-substituted or unsubstituted heteroalkyl, R⁴¹-substituted or unsubstituted cycloalkyl, R⁴¹-substituted or unsubstituted heterocycloalkyl, R⁴¹-substituted or unsubstituted aryl, or R⁴¹-substituted or unsubstituted heteroaryl; R^{4A} and R^{4B} substituents bonded to the same nitrogen atom may optionally be joined to form a R⁴¹-substituted or unsubstituted heterocycloalkyl or R⁴¹-substituted or unsubstituted heteroaryl; R^{4F} and R^{4G} substituents bonded to the same nitrogen atom may optionally be joined to form a R⁴¹-substituted or unsubstituted heterocycloalkyl or R⁴¹-substituted or unsubstituted heteroaryl.

In embodiments, R^{4A}, R^{4B}, R^{4C}, R^{4D}, R^{4F}, and R^{4G} are independently hydrogen, R¹-substituted or unsubstituted alkyl, R⁴¹-substituted or unsubstituted heteroalkyl, R⁴¹-substituted or unsubstituted cycloalkyl, R⁴¹-substituted or unsubstituted heterocycloalkyl, R⁴¹-substituted or unsubstituted aryl, or R⁴¹-substituted or unsubstituted heteroaryl; R^{4A} and R^{4B} substituents bonded to the same nitrogen atom may optionally be joined to form a R⁴¹-substituted or unsubstituted heterocycloalkyl or R⁴¹-substituted or unsubstituted heteroaryl; R^{4F} and R^{4G} substituents bonded to the same nitrogen atom may optionally be joined to form a R⁴¹-substituted or unsubstituted heterocycloalkyl or R⁴¹-substituted or unsubstituted heteroaryl.
R^{4E} is independently hydrogen, —C(O)R^{4F}, —C(O)—OR^{4F}, —SOR^{4F}, —SO₂R^{4F}, —C(O)NR^{4F}R^{G}, —SO₂NR^{4F}R^{G}; —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R⁴¹-substituted or unsubstituted alkyl (e.g., C₁-C₈, C₁-C₆, C₁-C₄, or C₁-C₂), R⁴¹-substituted or unsubstituted heteroalkyl (e.g., 2 to 8 membered, 2 to 6 membered, 4 to 6 membered, 2 to 3 membered, or 4 to 5 membered), R⁴¹-substituted or unsubstituted cycloalkyl (e.g., C₃-C₈, C₃-C₆, C₄-C₆, or C₅-C₆), R⁴¹-substituted or unsubstituted heterocycloalkyl (e.g., 3 to 8 membered, 3 to 6 membered, 4 to 6 membered, 4 to 5 membered, or 5 to 6 membered), R⁴¹-substituted or unsubstituted aryl (e.g., C₆-C₁₀ or phenyl), or R⁴¹-substituted or unsubstituted heteroaryl (e.g., 5 to 10 membered, 5 to 9 membered, or 5 to 6 membered).

In embodiments, R^{4E} is independently hydrogen, —C(O)R^{4F}, —C(O)—OR^{4F}, —SOR^{4F}, —SO₂R^{4F}, —C(O)NR^{4F}R^{G}, —SO₂NR^{4F}R^{G}, R⁴¹-substituted or unsubstituted alkyl (e.g., C₁-C₈, C₁-C₆, C₁-C₄, or C₁-C₂), R⁴¹-substituted or unsubstituted heteroalkyl (e.g., 2 to 8 membered, 2 to 6 membered, 4 to 6 membered, 2 to 3 membered, or 4 to 5 membered), R⁴¹-substituted or unsubstituted cycloalkyl (e.g., C₃-C₈, C₃-C₆, C₄-C₆, or C₅-C₆), R⁴¹-substituted or unsubstituted heterocycloalkyl (e.g., 3 to 8 membered, 3 to 6 membered, 4 to 6 membered, 4 to 5 membered, or 5 to 6 membered), R⁴¹-substituted or unsubstituted aryl (e.g., C₆-C₁₀ or phenyl), or R⁴¹-substituted or unsubstituted heteroaryl (e.g., 5 to 10 membered, 5 to 9 membered, or 5 to 6 membered).

In embodiments, R^{4E} is independently 1) hydrogen, —C(O)—OR^{4F}, —SOR^{4F}, —SO₂R^{4F}, —C(O)NR^{4F}R^{G}, —SO₂NR^{4F}R^{G}, R⁴¹-substituted or unsubstituted alkyl (e.g., C₁-C₈, C₁-C₆, C₁-C₄, or C₁-C₂), R⁴¹-substituted or unsubstituted heteroalkyl (e.g., 2 to 8 membered, 2 to 6 membered, 4 to 6 membered, 2 to 3 membered, or 4 to 5 membered), R⁴¹-substituted or unsubstituted cycloalkyl (e.g., C₃-C₈, C₃-C₆, C₄-C₆, or C₅-C₆), R⁴¹-substituted or unsubstituted heterocycloalkyl (e.g., 3 to 8 membered, 3 to 6 membered, 4 to 6 membered, 4 to 5 membered, or 5 to 6 membered), R⁴¹-substituted or unsubstituted aryl (e.g., C₆—C₁₀ or phenyl), or R⁴-substituted or unsubstituted heteroaryl (e.g., 5 to 10 membered, 5 to 9 membered, or 5 to 6 membered); wherein R^{4F} and R^{4G} are independently hydrogen, R⁴¹-substituted or unsubstituted alkyl (e.g., C₁-C₈, C₁-C₆, C₁-C₄, or C₁-C₂), R⁴¹-substituted or unsubstituted heteroalkyl (e.g., 2 to 8 membered, 2 to 6 membered, 4 to 6 membered, 2 to 3 membered, or 4 to 5 membered), R⁴¹-substituted or unsubstituted cycloalkyl (e.g., C₃-C₈, C₃-C₆, C₄-C₆, or C₅-C₆), R⁴¹-substituted or unsubstituted heterocycloalkyl (e.g., 3 to 8 membered, 3 to 6 membered, 4 to 6 membered, 4 to 5 membered, or 5 to 6 membered), R⁴¹-substituted or unsubstituted aryl (e.g., C₆-C₁₀ or phenyl), or R⁴-substituted or unsubstituted heteroaryl (e.g., 5 to 10 membered, 5 to 9 membered, or 5 to 6 membered); R^{4A} and R^{4B} substituents bonded to the same nitrogen atom may optionally be joined to form a R⁴¹-substituted or unsubstituted heterocycloalkyl (e.g., 3 to 8 membered, 3 to 6 membered, 4 to 6 membered, 4 to 5 membered, or 5 to 6 membered) or R⁴-substituted or unsubstituted heteroaryl (e.g., 5 to 10 membered, 5 to 9 membered, or 5 to 6 membered); R^{4F} and R^{4G} substituents bonded to the same nitrogen atom may optionally be joined to form a R⁴¹-substituted or unsubstituted heterocycloalkyl (e.g., 3 to 8 membered, 3 to 6 membered, 4 to 6 membered, 4 to 5 membered, or 5 to 6 membered) or R⁴¹ substituted or unsubstituted heteroaryl (e.g., 5 to 10 membered, 5 to 9 membered, or 5 to 6 membered); or 2) —C(O)R^{4F}; wherein R^{4F} is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R⁴¹-substituted or unsubstituted alkyl (e.g., C₁-C₈, C₁-C₆, C₁-C₄, or C₁-C₂), R⁴¹-substituted or unsubstituted heteroalkyl (e.g., 2 to 8 membered, 2 to 6 membered, 4 to 6 membered, 2 to 3 membered, or 4 to 5 membered), R⁴¹-substituted or unsubstituted cycloalkyl (e.g., C₃-C₈, C₃-C₆, C₄-C₆, or C₅-C₆), R⁴¹-substituted or unsubstituted heterocycloalkyl (e.g., 3 to 8 membered, 3 to 6 membered, 4 to 6 membered, 4 to 5 membered, or 5 to 6 membered), R⁴¹-substituted or unsubstituted aryl (e.g., C₆-C₁₀ or phenyl), or R⁴-substituted or unsubstituted heteroaryl (e.g., 5 to 10 membered, 5 to 9 membered, or 5 to 6 membered).

In embodiments, R^{4E} is independently hydrogen, —C(O)R^{4F}, —C(O)—OR^{4F}, —SOR^{4F}, —SO₂R^{4F}, —C(O)NR^{4F}R^{4G}, —SO₂NR^{4F}R^{4G}, —CF₃, —CHF₂, —CH₂F, —CN, —OH, —NH₂, —COOH, —CONH₂, —OCF₃, —OCHF₂, —OCH₂F, R⁴¹-substituted or unsubstituted alkyl (e.g., C₁-C₈, C₁-C₆, C₁-C₄, or C₁-C₂), R⁴¹-substituted or unsubstituted heteroalkyl (e.g., 2 to 8 membered, 2 to 6 membered, 4 to 6 membered, 2 to 3 membered, or 4 to 5 membered), R⁴¹-substituted or unsubstituted cycloalkyl (e.g., C₃-C₈, C₃-C₆, C₄-C₆, or C₅-C₆), R⁴¹-substituted or unsubstituted heterocycloalkyl (e.g., 3 to 8 membered, 3 to 6 membered, 4 to 6 membered, 4 to 5 membered, or 5 to 6 membered), R⁴¹-substituted or unsubstituted aryl (e.g., C₆-C₁₀ or phenyl), or R⁴¹-substituted or unsubstituted heteroaryl (e.g., 5 to 10 membered, 5 to 9 membered, or 5 to 6 membered).
R⁴¹ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, R⁴²-substituted or unsubstituted saturated alkyl (e.g., C₁-C₈, C₁-C₆, C₁-C₄, or C₁-C₂), R⁴²-substituted or unsubstituted alkenyl (e.g., C₂-C₈, C₂-C₆, C₂-C₄, or C₂), R⁴²-substituted or unsubstituted alkynyl (e.g., C₂-C₈, C₂-C₆, C₂-C₄, or C₂), R⁴²-substituted or unsubstituted heteroalkyl (e.g., 2 to 12 membered, 2 to 6 membered, 4 to 6 membered, 2 to 3 membered, or 4 to 5 membered), R⁴²-substituted or unsubstituted cycloalkyl (e.g., C₃-C₈, C₃-C₆, C₄-C₆, or C₅-C₆), R⁴²-substituted or unsubstituted heterocycloalkyl (e.g., 3 to 15 membered, 3 to 6 membered, 4 to 6 membered, 4 to 5 membered, or 5 to 6 membered), R⁴²-substituted or unsubstituted aryl (e.g., C₆-C₁₀ or phenyl), or R⁴²-substituted or unsubstituted heteroaryl (e.g., 5 to 10 membered, 5 to 9 membered, or 5 to 6 membered).

In embodiments, R⁴¹ is independently oxo, halogen, —CF₃, —CHF₂, —CH₂F, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSO₂H, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, —OCH₂F, —SF₅, —N₃, —O—P(O)(OH)₂, R⁴²-substituted or unsubstituted saturated alkyl (e.g., C₁-C₈, C₁-C₆, C₁-C₄, or C₁-C₂), R⁴²-substituted or unsubstituted alkenyl (e.g., C₂-C₈, C₂-C₆, C₂-C₄, or C₂), R⁴²-substituted or unsubstituted alkynyl (e.g., C₂-C₈, C₂-C₆, C₂-C₄, or C₂), R⁴²-substituted or unsubstituted heteroalkyl (e.g., 2 to 8 membered, 2 to 6 membered, 4 to 6 membered, 2 to 3 membered, or 4 to 5 membered), R⁴²-substituted or unsubstituted cycloalkyl (e.g., C₃-C₈, C₃-C₆, C₄-C₆, or C₅-C₆), R⁴²-substituted or unsubstituted heterocycloalkyl (e.g., 3 to 8 membered, 3 to 6 membered, 4 to 6 membered, 4 to 5 membered, or 5 to 6 membered), R⁴²-substituted or unsubstituted aryl (e.g., C₆-C₁₀ or phenyl), or R⁴²-substituted or unsubstituted heteroaryl (e.g., 5 to 10 membered, 5 to 9 membered, or 5 to 6 membered).

In embodiments, R⁴¹ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, R⁴²-substituted or unsubstituted C₁-C₄ saturated alkyl, R⁴²-substituted or unsubstituted C₂-C₄ alkenyl, R⁴²-substituted or unsubstituted C₂-C₄ alkynyl, R⁴²-substituted or unsubstituted 2 to 12 membered heteroalkyl, R⁴²-substituted or unsubstituted C₃-C₆ cycloalkyl, R⁴²-substituted or unsubstituted 3 to 15 membered heterocycloalkyl, R⁴²-substituted or unsubstituted phenyl, or R⁴²-substituted or unsubstituted 5 to 6 membered heteroaryl.
R⁴² is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, R⁴³-substituted or unsubstituted saturated alkyl (e.g., C₁-C₈, C₁-C₆, C₁-C₄, or C₁-C₂), R⁴³-substituted or unsubstituted alkenyl (e.g., C₂-C₈, C₂-C₆, C₂-C₄, or C₂), R⁴³-substituted or unsubstituted alkynyl (e.g., C₂-C₈, C₂-C₆, C₂-C₄, or C₂), R⁴³-substituted or unsubstituted heteroalkyl (e.g., 2 to 12 membered, 2 to 6 membered, 4 to 6 membered, 2 to 3 membered, or 4 to 5 membered), R⁴³-substituted or unsubstituted cycloalkyl (e.g., C₃-C₈, C₃-C₆, C₄-C₆, or C₅-C₆), R⁴³-substituted or unsubstituted heterocycloalkyl (e.g., 3 to 15 membered, 3 to 6 membered, 4 to 6 membered, 4 to 5 membered, or 5 to 6 membered), R⁴³-substituted or unsubstituted aryl (e.g., C₆-C₁₀ or phenyl), or R⁴³-substituted or unsubstituted heteroaryl (e.g., 5 to 10 membered, 5 to 9 membered, or 5 to 6 membered).

In embodiments, R⁴² is independently oxo, halogen, —CF₃, —CHF₂, —CH₂F, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSO₂H, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, —OCH₂F, —SF₅, —N₃, —O—P(O)(OH)₂, R⁴³-substituted or unsubstituted saturated alkyl (e.g., C₁-C₈, C₁-C₆, C₁-C₄, or C₁-C₂), R⁴³-substituted or unsubstituted alkenyl (e.g., C₂-C₈, C₂-C₆, C₂-C₄, or C₂), R⁴³-substituted or unsubstituted alkynyl (e.g., C₂-C₈, C₂-C₆, C₂-C₄, or C₂), R⁴³-substituted or unsubstituted heteroalkyl (e.g., 2 to 8 membered, 2 to 6 membered, 4 to 6 membered, 2 to 3 membered, or 4 to 5 membered), R⁴³-substituted or unsubstituted cycloalkyl (e.g., C₃-C₈, C₃-C₆, C₄-C₆, or C₅-C₆), R⁴³-substituted or unsubstituted heterocycloalkyl (e.g., 3 to 8 membered, 3 to 6 membered, 4 to 6 membered, 4 to 5 membered, or 5 to 6 membered), R⁴³-substituted or unsubstituted aryl (e.g., C₆-C₁₀ or phenyl), or R⁴³-substituted or unsubstituted heteroaryl (e.g., 5 to 10 membered, 5 to 9 membered, or 5 to 6 membered).

In embodiments, R⁴² is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, R⁴³-substituted or unsubstituted C₁-C₄ saturated alkyl, R⁴³-substituted or unsubstituted C₂-C₄ alkenyl, R⁴³-substituted or unsubstituted C₂-C₄ alkynyl, R⁴³-substituted or unsubstituted 2 to 12 membered heteroalkyl, R⁴³-substituted or unsubstituted C₃-C₆ cycloalkyl, R⁴³-substituted or unsubstituted 3 to 15 membered heterocycloalkyl, R⁴³-substituted or unsubstituted phenyl, or R⁴³-substituted or unsubstituted 5 to 6 membered heteroaryl.
R⁴³ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, R⁴³ is independently oxo, halogen, —CF₃, —CHF₂, —CH₂F, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSO₂H, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, —OCH₂F, —SF₅, —N₃, —O—P(O)(OH)₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, L is R^{L}-substituted or unsubstituted C₁-C₁₀ saturated alkylene, R^{L}-substituted or unsubstituted C₂-C₁₀ alkenylene, R^{L}-substituted or unsubstituted C₂-C₁₀ alkynylene, R^{L}-substituted or unsubstituted C₂-C₆ alkylene-O—C₂-C₆ alkylene, R^{L}-substituted or unsubstituted C₂ alkylene-O—C₂ alkylene, R^{L}-substituted or unsubstituted C₂ alkylene-O—C₂ alkylene-O—C₂ alkylene, R^{L}-substituted or unsubstituted C₂-C₆ alkylene-N(R^{L4})—C₂-C₆ alkylene, R^{L}-substituted or unsubstituted C₃-C₆ cycloalkylene, R^{L}-substituted or unsubstituted phenylene, R^{L}-substituted or unsubstituted 4-6 membered heterocycloalkylene, R^{L}-substituted or unsubstituted 5-6 membered heteroarylene, R^{L}-substituted or unsubstituted C₁-C₄alkylene-(C₃-C₆ cycloalkylene)-C₁-C₄alkylene, R^{L}-substituted or unsubstituted C₁-C₄ alkylene-phenylene-C₁-C₄ alkylene, R^{L}-substituted or unsubstituted C₁-C₄ alkylene-(4-6 membered heterocycloalkylene)-C₁-C₄ alkylene, or R^{L}-substituted or unsubstituted C₁-C₄ alkylene-(5-6 membered heteroarylene)-C₁-C₄ alkylene.
R^{L} is halogen, —OR^{LB}, —O—P(O)(OR^{LA})₂, -(unsubstituted alkyl)-O—P(O)(OR^{LA})₂, -(unsubstituted alkoxy)-O—P(O)(OR^{LA})₂, —NR^{LA}R^{LB}, —OC(O)R^{LB}, —C(O)—OR^{LB}, —SOR^{LB}, —SO₂R^{LB}, —C(O)NR^{LA}R^{LB}, —SO₂NR^{LA}R^{LB}, —OC(O)NR^{LA}R^{LB}, —NR^{LA}C(O)R^{LB}, —NR^{LA}SOR^{LB}, —NR^{LA}C(O)OR^{LB}, —NR^{LA}SO₂R^{LB}, —R^{LA} SO₂R^{LB}, —CN, —CX^{L}₃, —CHX^{L}₂, -CH₂XL, —C(O)R^{LB}, —OCX^{L}₃, —OCH₂X^{L}, —OCHX^{L}₂, —SR^{LB}, —NHNR^{LA}R^{LB}, —ONR^{LA}R^{LB}, —NHC(O)NHNR^{LA}R^{LB}, —NHC(O)NR^{LA}R^{LB}, —N(O)₂, —NR^{LA}OR^{LB}, —SF₅, —N₃, R^{L6}-substituted or unsubstituted saturated alkyl (e.g., C₁-C₈, C₁-C₆, C₁-C₄, or C₁-C₂), R^{L}-substituted or unsubstituted alkenyl (e.g., C₂-C₈, C₂-C₆, C₂-C₄, or C₂), R^{L6}-substituted or unsubstituted alkynyl (e.g., C₂-C₈, C₂-C₆, C₂-C₄, or C₂), R^{L6}-substituted or unsubstituted heteroalkyl (e.g., 2 to 12 membered, 2 to 6 membered, 4 to 6 membered, 2 to 3 membered, or 4 to 5 membered), R^{L6}-substituted or unsubstituted cycloalkyl (e.g., C₃-C₈, C₃-C₆, C₄-C₆, or C₅-C₆), R^{L6}-substituted or unsubstituted heterocycloalkyl (e.g., 3 to 15 membered, 3 to 6 membered, 4 to 6 membered, 4 to 5 membered, or 5 to 6 membered), R^{L6}-substituted or unsubstituted aryl (e.g., C₆-C₁₀ or phenyl), or R^{L6}-substituted or unsubstituted heteroaryl (e.g., 5 to 10 membered, 5 to 9 membered, or 5 to 6 membered). X^{L} is independently —F, —Cl, —Br, or —I.

In embodiments, R^{L} is halogen, —OR^{LB}, —O—P(O)(OR^{LA})₂, -(unsubstituted alkyl)-O—P(O)(OR^{LA})₂, -(unsubstituted alkoxy)-O—P(O)(OR^{LA})₂, —NR^{LA}R^{LB}, —OC(O)R^{LB}, —C(O)—OR^{LB}, —SOR^{LB}, —SO₂R^{LB}, —C(O)NR^{LA}R^{LB}, —SO₂NR^{LA}R^{LB}, —OC(O)NR^{LA}R^{LB}, —NR^{LA}C(O)R^{LB}, —NR^{LA}SOR^{LB}, —NR^{LA}C(O)OR^{LB}, —NR^{LA}SO₂R^{LB}, —R^{LA} SO₂R^{LB}, —CN, —CF₃, —CHF₂, —CH₂F, —C(O)R^{LB}, —OCF₃, —OCH₂F, —OCHF₂, —SR^{LB}, —NHNR^{LA}R^{LB}, —ONR^{LA}R^{LB}, —NHC(O)NHNR^{LA}R^{LB}, —NHC(O)NR^{LA}R^{LB}, —N(O)₂, —NR^{LA}OR^{LB}, —SF₅, —N₃, R^{L6}-substituted or unsubstituted saturated alkyl (e.g., C₁-C₈, C₁-C₆, C₁-C₄, or C₁-C₂), R^{L6}-substituted or unsubstituted alkenyl (e.g., C₂-C₈, C₂-C₆, C₂-C₄, or C₂), R^{L6}-substituted or unsubstituted alkynyl (e.g., C₂-C₈, C₂-C₆, C₂-C₄, or C₂), R^{L6}-substituted or unsubstituted heteroalkyl (e.g., 2 to 8 membered, 2 to 6 membered, 4 to 6 membered, 2 to 3 membered, or 4 to 5 membered), R^{L6}-substituted or unsubstituted cycloalkyl (e.g., C₃-C₈, C₃-C₆, C₄-C₆, or C₅-C₆), R′-substituted or unsubstituted heterocycloalkyl (e.g., 3 to 8 membered, 3 to 6 membered, 4 to 6 membered, 4 to 5 membered, or 5 to 6 membered), R^{L6}-substituted or unsubstituted aryl (e.g., C₆-C₁₀ or phenyl), or R^{L6}-substituted or unsubstituted heteroaryl (e.g., 5 to 10 membered, 5 to 9 membered, or 5 to 6 membered); R^{LA} and R^{LB} substituents bonded to the same nitrogen atom may optionally be joined to form a R^{L6}-substituted or unsubstituted heterocycloalkyl (e.g., 3 to 8 membered, 3 to 6 membered, 4 to 6 membered, 4 to 5 membered, or 5 to 6 membered) or R^{L6}-substituted or unsubstituted heteroaryl (e.g., 5 to 10 membered, 5 to 9 membered, or 5 to 6 membered).

In embodiments, R^{L} is halogen, —OR^{LB}, —O—P(O)(OR^{LA})₂, -(unsubstituted alkyl)-O—P(O)(OR^{LA})₂, -(unsubstituted alkoxy)-O—P(O)(OR^{LA})₂, —NR^{LA}R^{LB}, —C(O)—OR^{LB}, —SOR^{LB}, —SO₂R^{LB}, —C(O)NR^{LA}R^{LB}, —SO₂NR^{LA}R^{LB}, —OC(O)NR^{LA}R^{LB}, —NR^{LA}SOR^{LB}, —NR^{LA}C(O)OR^{LB}, —NR^{LA}SO₂R^{LB}, —R^{LA}SO₂R^{LB}, —CN, —CF₃, —CHF₂, —CH₂F, —OCF₃, —OCH₂F, —OCHF₂, —SR^{LB}, —NHR^{LA}R^{LB}, —ONR^{LA}R^{LB}, —NHC(O)NHNR^{LA}R^{LB}, —NHC(O)NR^{LA}R^{LB}, —N(O)₂, —NR^{LA}OR^{LB}, —SF₅, —N₃, R^{L6}-substituted or unsubstituted saturated alkyl (e.g., C₁-C₈, C₁-C₆, C₁-C₄, or C₁-C₂), R^{L1}-substituted or unsubstituted alkenyl (e.g., C₂-C₈, C₂-C₆, C₂-C₄, or C₂), R^{L6}-substituted or unsubstituted alkynyl (e.g., C₂-C₈, C₂-C₆, C₂-C₄, or C₂), R^{L6}-substituted or unsubstituted heteroalkyl (e.g., 2 to 8 membered, 2 to 6 membered, 4 to 6 membered, 2 to 3 membered, or 4 to 5 membered), R-substituted or unsubstituted cycloalkyl (e.g., C₃-C₈, C₃-C₆, C₄-C₆, or C₅-C₆), R^{L6}-substituted or unsubstituted heterocycloalkyl (e.g., 3 to 8 membered, 3 to 6 membered, 4 to 6 membered, 4 to 5 membered, or 5 to 6 membered), R^{L6}-substituted or unsubstituted aryl (e.g., C₆-C₁₀ or phenyl), or R^{L6}-substituted or unsubstituted heteroaryl (e.g., 5 to 10 membered, 5 to 9 membered, or 5 to 6 membered); wherein R^{LA} and R^{LB} are independently hydrogen, R^{L6}-substituted or unsubstituted saturated alkyl (e.g., C₁-C₈, C₁-C₆, C₁-C₄, or C₁-C₂), R^{L6}-substituted or unsubstituted alkenyl (e.g., C₂-C₈, C₂-C₆, C₂-C₄, or C₂), R^{L6}-substituted or unsubstituted alkynyl (e.g., C₂-C₈, C₂-C₆, C₂-C₄, or C₂), R^{L6}-substituted or unsubstituted heteroalkyl (e.g., 2 to 8 membered, 2 to 6 membered, 4 to 6 membered, 2 to 3 membered, or 4 to 5 membered), R^{L6}-substituted or unsubstituted cycloalkyl (e.g., C₃-C₈, C₃-C₆, C₄-C₆, or C₅-C₆), R^{L6}-substituted or unsubstituted heterocycloalkyl (e.g., 3 to 8 membered, 3 to 6 membered, 4 to 6 membered, 4 to 5 membered, or 5 to 6 membered), R^{L6}-substituted or unsubstituted aryl (e.g., C₆-C₁₀ or phenyl), or R^{L6}-substituted or unsubstituted heteroaryl (e.g., 5 to 10 membered, 5 to 9 membered, or 5 to 6 membered); R^{LA} and R^{LB} substituents bonded to the same nitrogen atom may optionally be joined to form a R^{L6}-substituted or unsubstituted heterocycloalkyl (e.g., 3 to 8 membered, 3 to 6 membered, 4 to 6 membered, 4 to 5 membered, or 5 to 6 membered) or R^{L6}-substituted or unsubstituted heteroaryl (e.g., 5 to 10 membered, 5 to 9 membered, or 5 to 6 membered); or 2) —OC(O)R^{LB}, —NR^{LA}C(O)R^{LB}, or —C(O)R^{LB}; wherein R^{LA} is independently hydrogen, R^{L6}-substituted or unsubstituted saturated alkyl (e.g., C₁-C₈, C₁-C₆, C₁-C₄, or C₁-C₂), R^{L6}-substituted or unsubstituted alkenyl (e.g., C₂-C₈, C₂-C₆, C₂-C₄, or C₂), R^{L6}-substituted or unsubstituted alkynyl (e.g., C₂-C₈, C₂-C₆, C₂-C₄, or C₂), R^{L6}-substituted or unsubstituted heteroalkyl (e.g., 2 to 8 membered, 2 to 6 membered, 4 to 6 membered, 2 to 3 membered, or 4 to 5 membered), R-substituted or unsubstituted cycloalkyl (e.g., C₃-C₈, C₃-C₆, C₄-C₆, or C₅-C₆), R^{L6}-substituted or unsubstituted heterocycloalkyl (e.g., 3 to 8 membered, 3 to 6 membered, 4 to 6 membered, 4 to 5 membered, or 5 to 6 membered), R^{L6}-substituted or unsubstituted aryl (e.g., C₆-C₁₀ or phenyl), or R^{L6}-substituted or unsubstituted heteroaryl (e.g., 5 to 10 membered, 5 to 9 membered, or 5 to 6 membered); R^{LB} is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —COH, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R-substituted or unsubstituted saturated alkyl (e.g., C₁-C₈, C₁-C₆, C₁-C₄, or C₁-C₂), R^{L6}-substituted or unsubstituted alkenyl (e.g., C₂-C₈, C₂-C₆, C₂-C₄, or C₂), R^{L6}-substituted or unsubstituted alkynyl (e.g., C₂-C₈, C₂-C₆, C₂-C₄, or C₂), R^{L6}-substituted or unsubstituted heteroalkyl (e.g., 2 to 8 membered, 2 to 6 membered, 4 to 6 membered, 2 to 3 membered, or 4 to 5 membered), R^{L6}-substituted or unsubstituted cycloalkyl (e.g., C₃-C₈, C₃-C₆, C₄-C₆, or C₅-C₆), R^{L6}-substituted or unsubstituted heterocycloalkyl (e.g., 3 to 8 membered, 3 to 6 membered, 4 to 6 membered, 4 to 5 membered, or 5 to 6 membered), R^{L6}-substituted or unsubstituted aryl (e.g., C₆-C₁₀ or phenyl), or R^{L6}-substituted or unsubstituted heteroaryl (e.g., 5 to 10 membered, 5 to 9 membered, or 5 to 6 membered).

In embodiments, R^{L} is halogen, —OR^{LB}, —O—P(O)(OR^{LA})₂, -(unsubstituted alkyl)-O—P(O)(OR^{LA})₂, -(unsubstituted alkoxy)-O—P(O)(OR^{LA})₂, —NR^{LA}R^{LB}, —OC(O)R^{LB}, —C(O)—OR^{LB}, —SOR^{LB}, —SO₂R^{LB}, —C(O)NR^{LA}R^{LB}, —SO₂NR^{LA}R^{LB}, —OC(O)NR^{LA}R^{LB}, —NR^{LA}C(O)R^{LB}, —NR^{LA}SOR^{LB}, —NR^{LA}C(O)OR^{LB}, —NR^{LA}SO₂R^{LB}, —R^{LA} SO₂R^{LB}, —CN, —CX^{L}₃, —CHX^{L}₂, —CH₂X^{L}, —C(O)R^{LB}, —OCX^{L}₃, —OCH₂X^{L}, —OCHX^{L}₂, —SR^{LB}, —NHNR^{LA}R^{LB}, —ONR^{LA}R^{LB}, —NHC(O)NHNR^{LA}R^{LB}, —NHC(O)NR^{LA}R^{LB}, —N(O)₂, —NR^{LA}OR^{LB}, —SF₅, —N₃, R^{L6}-substituted or unsubstituted C₁-C₆ saturated alkyl, R^{L6}-substituted or unsubstituted C₂-C₆ alkenyl, R′-substituted or unsubstituted C₂-C₆ alkynyl, R^{L6}-substituted or unsubstituted 2 to 12 membered heteroalkyl, R^{L6}-substituted or unsubstituted cycloalkyl, R^{L6}-substituted or unsubstituted heterocycloalkyl, R^{L6}-substituted or unsubstituted aryl, or R^{L6}-substituted or unsubstituted heteroaryl. X^{L} is independently —F, —Cl, —Br, or —I
R^{LA} and R^{LB} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —COH, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R^{L6}-substituted or unsubstituted saturated alkyl (e.g., C₁-C₈, C₁-C₆, C₁-C₄, or C₁-C₂), R^{L6}-substituted or unsubstituted alkenyl (e.g., C₂-C₈, C₂-C₆, C₂-C₄, or C₂), R^{L6}-substituted or unsubstituted alkynyl (e.g., C₂-C₈, C₂-C₆, C₂-C₄, or C₂), R^{L6}-substituted or unsubstituted heteroalkyl (e.g., 2 to 8 membered, 2 to 6 membered, 4 to 6 membered, 2 to 3 membered, or 4 to 5 membered), R′-substituted or unsubstituted cycloalkyl (e.g., C₃-C₈, C₃-C₆, C₄-C₆, or C₅-C₆), R^{L6}-substituted or unsubstituted heterocycloalkyl (e.g., 3 to 8 membered, 3 to 6 membered, 4 to 6 membered, 4 to 5 membered, or 5 to 6 membered), R^{L6}-substituted or unsubstituted aryl (e.g., C₆-C₁₀ or phenyl), or R^{L6}-substituted or unsubstituted heteroaryl (e.g., 5 to 10 membered, 5 to 9 membered, or 5 to 6 membered); R^{LA} and R^{LB} substituents bonded to the same nitrogen atom may optionally be joined to form a R^{L6}-substituted or unsubstituted heterocycloalkyl (e.g., 3 to 8 membered, 3 to 6 membered, 4 to 6 membered, 4 to 5 membered, or 5 to 6 membered) or R^{L}-substituted or unsubstituted heteroaryl (e.g., 5 to 10 membered, 5 to 9 membered, or 5 to 6 membered).

In embodiments, R^{LA} and R^{LB} are independently hydrogen, R^{L6}-substituted or unsubstituted saturated alkyl (e.g., C₁-C₈, C₁-C₆, C₁-C₄, or C₁-C₂), R^{L6}-substituted or unsubstituted alkenyl (e.g., C₂-C₈, C₂-C₆, C₂-C₄, or C₂), R^{L6}-substituted or unsubstituted alkynyl (e.g., C₂-C₈, C₂-C₆, C₂-C₄, or C₂), R^{L6}-substituted or unsubstituted heteroalkyl (e.g., 2 to 8 membered, 2 to 6 membered, 4 to 6 membered, 2 to 3 membered, or 4 to 5 membered), R^{L6}-substituted or unsubstituted cycloalkyl (e.g., C₃-C₈, C₃-C₆, C₄-C₆, or C₅-C₆), R^{L6}-substituted or unsubstituted heterocycloalkyl (e.g., 3 to 8 membered, 3 to 6 membered, 4 to 6 membered, 4 to 5 membered, or 5 to 6 membered), R^{L6}-substituted or unsubstituted aryl (e.g., C₆-C₁₀ or phenyl), or R^{L6}-substituted or unsubstituted heteroaryl (e.g., 5 to 10 membered, 5 to 9 membered, or 5 to 6 membered); R^{LA} and R^{LB} substituents bonded to the same nitrogen atom may optionally be joined to form a R^{L6}-substituted or unsubstituted heterocycloalkyl (e.g., 3 to 8 membered, 3 to 6 membered, 4 to 6 membered, 4 to 5 membered, or 5 to 6 membered) or R^{L6}-substituted or unsubstituted heteroaryl (e.g., 5 to 10 membered, 5 to 9 membered, or 5 to 6 membered).

In embodiments, R^{LA} and R^{LB} are independently hydrogen, —CF₃, —CHF₂, —CH₂F, —CN, —OH, —COH, —COOH, —CONH₂, —OCF₃, —OCHF₂, —OCH₂F, R^{L6}-substituted or unsubstituted saturated alkyl (e.g., C₁-C₈, C₁-C₆, C₁-C₄, or C₁-C₂), R^{L6}-substituted or unsubstituted alkenyl (e.g., C₂-C₈, C₂-C₆, C₂-C₄, or C₂), R^{L6}-substituted or unsubstituted alkynyl (e.g., C₂-C₈, C₂-C₆, C₂-C₄, or C₂), R^{L6}-substituted or unsubstituted heteroalkyl (e.g., 2 to 8 membered, 2 to 6 membered, 4 to 6 membered, 2 to 3 membered, or 4 to 5 membered), R^{L6}-substituted or unsubstituted cycloalkyl (e.g., C₃-C₈, C₃-C₆, C₄-C₆, or C₅-C₆), R^{L6}-substituted or unsubstituted heterocycloalkyl (e.g., 3 to 8 membered, 3 to 6 membered, 4 to 6 membered, 4 to 5 membered, or 5 to 6 membered), R^{L6}-substituted or unsubstituted aryl (e.g., C₆-C₁₀ or phenyl), or R^{L6}-substituted or unsubstituted heteroaryl (e.g., 5 to 10 membered, 5 to 9 membered, or 5 to 6 membered); R^{LA} and R^{LB} substituents bonded to the same nitrogen atom may optionally be joined to form a R^{L6}-substituted or unsubstituted heterocycloalkyl (e.g., 3 to 8 membered, 3 to 6 membered, 4 to 6 membered, 4 to 5 membered, or 5 to 6 membered) or R^{L6}-substituted or unsubstituted heteroaryl (e.g., 5 to 10 membered, 5 to 9 membered, or 5 to 6 membered).
R^{L6} is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, R^{L7}-substituted or unsubstituted saturated alkyl (e.g., C₁-C₈, C₁-C₆, C₁-C₄, or C₁-C₂), R^{L7}-substituted or unsubstituted alkenyl (e.g., C₂-C₈, C₂-C₆, C₂-C₄, or C₂), R^{L7}-substituted or unsubstituted alkynyl (e.g., C₂-C₈, C₂-C₆, C₂-C₄, or C₂), R^{L7}-substituted or unsubstituted heteroalkyl (e.g., 2 to 12 membered, 2 to 6 membered, 4 to 6 membered, 2 to 3 membered, or 4 to 5 membered), R^{L7}-substituted or unsubstituted cycloalkyl (e.g., C₃-C₈, C₃-C₆, C₄-C₆, or C₅-C₆), R^{L7}-substituted or unsubstituted heterocycloalkyl (e.g., 3 to 15 membered, 3 to 6 membered, 4 to 6 membered, 4 to 5 membered, or 5 to 6 membered), R^{L7}-substituted or unsubstituted aryl (e.g., C₆-C₁₀ or phenyl), or R^{L7}-substituted or unsubstituted heteroaryl (e.g., 5 to 10 membered, 5 to 9 membered, or 5 to 6 membered).

In embodiments, R^{L6} is independently oxo, halogen, —CF₃, —CHF₂, —CH₂F, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSO₂H, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, —OCH₂F, —SF₅, —N₃, —O—P(O)(OH)₂, R^{L7}-substituted or unsubstituted saturated alkyl (e.g., C₁-C₈, C₁-C₆, C₁-C₄, or C₁-C₂), R^{L7}-substituted or unsubstituted alkenyl (e.g., C₂-C₈, C₂-C₆, C₂-C₄, or C₂), R^{L7}-substituted or unsubstituted alkynyl (e.g., C₂-C₈, C₂-C₆, C₂-C₄, or C₂), R^{L7}-substituted or unsubstituted heteroalkyl (e.g., 2 to 8 membered, 2 to 6 membered, 4 to 6 membered, 2 to 3 membered, or 4 to 5 membered), R^{L7}-substituted or unsubstituted cycloalkyl (e.g., C₃-C₈, C₃-C₆, C₄-C₆, or C₅-C₆), R^{L7}-substituted or unsubstituted heterocycloalkyl (e.g., 3 to 8 membered, 3 to 6 membered, 4 to 6 membered, 4 to 5 membered, or 5 to 6 membered), R^{L7}-substituted or unsubstituted aryl (e.g., C₆-C₁₀ or phenyl), or R^{L7}-substituted or unsubstituted heteroaryl (e.g., 5 to 10 membered, 5 to 9 membered, or 5 to 6 membered).

In embodiments, R^{L6} is independently oxo, halogen, —CCl₃, —CBr₃, —CF₃, —CI₃, —CHCl₂, —CHBr₂, —CHF₂, —CHI₂, —CH₂C₁, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂C₁, —OCH₂Br, —OCH₂I, —OCH₂F, —SF₅, —N₃, —O—P(O)(OH)₂, R^{L7}-substituted or unsubstituted C₁-C₄ saturated alkyl, R^{L7}-substituted or unsubstituted C₂-C₄ alkenyl, R^{L7}-substituted or unsubstituted C₂-C₄ alkynyl, R^{L7}-substituted or unsubstituted 2 to 12 membered heteroalkyl, R^{L7}-substituted or unsubstituted C₃-C₆ cycloalkyl, R^{L7}-substituted or unsubstituted 3 to 15 membered heterocycloalkyl, R^{L7}-substituted or unsubstituted phenyl, or R^{L7}-substituted or unsubstituted 5 to 6 membered heteroaryl.
R^{L7} is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or s unsubstituted heteroaryl (e.g., 5 to 10 membered, 5 to 9 membered, or 5 to 6 membered).

In embodiments, R^{L7} is independently oxo, halogen, —CF₃, —CHF₂, —CH₂F, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSO₂H, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, —OCH₂F, —SF₅, —N₃, —O—P(O)(OH)₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or s unsubstituted heteroaryl (e.g., 5 to 10 membered, 5 to 9 membered, or 5 to 6 membered).

In embodiments, R^{L4} is independently hydrogen, COR^{L4A}, CO₂H, CO₂R^{L4A}, SOR^{L4A}, SO₂R^{L4A}, CONH₂, CONR^{L4A}R^{L4B}, SO₂NH₂, or SO₂NR^{L4A}R^{L4B}; R^{L6}-substituted or unsubstituted alkyl (e.g., C₁-C₈, C₁-C₆, C₁-C₄, or C₁-C₂), R^{L6}-substituted or unsubstituted heteroalkyl (e.g., 2 to 8 membered, 2 to 6 membered, 4 to 6 membered, 2 to 3 membered, or 4 to 5 membered), R^{L6}-substituted or unsubstituted cycloalkyl (e.g., C₃-C₈, C₃-C₆, C₄-C₆, or C₅-C₆), R^{L6}-substituted or unsubstituted heterocycloalkyl (e.g., 3 to 8 membered, 3 to 6 membered, 4 to 6 membered, 4 to 5 membered, or 5 to 6 membered), R^{L6}-substituted or unsubstituted aryl (e.g., C₆-C₁₀ or phenyl), or R^{L6}-substituted or unsubstituted heteroaryl (e.g., 5 to 10 membered, 5 to 9 membered, or 5 to 6 membered).

In embodiments, R^{L4A} and R^{L4B} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R^{L6}-substituted or unsubstituted alkyl, R^{L6}-substituted or unsubstituted heteroalkyl, R^{L6}-substituted or unsubstituted cycloalkyl, R^{L6}-substituted or unsubstituted heterocycloalkyl, R^{L6}-substituted or unsubstituted aryl, or R^{L6}-substituted or unsubstituted heteroaryl; R^{L4A} and R^{L4B} substituents bonded to the same nitrogen atom may optionally be joined to form a R^{L6}-substituted or unsubstituted heterocycloalkyl or R^{L6}-substituted or unsubstituted heteroaryl.

In embodiments, R^{L1} is independently hydrogen, unsubstituted C₁-C₆ saturated alkyl, unsubstituted C₂-C₆ alkenyl, unsubstituted C₂-C₆ alkynyl, unsubstituted 2 to 6 membered heteroalkyl, unsubstituted C₃-C₆ cycloalkyl, or unsubstituted 3 to 6 membered heterocycloalkyl;

In embodiments, R^{L2} is independently hydrogen, unsubstituted C₁-C₆ saturated alkyl, unsubstituted C₂-C₆ alkenyl, unsubstituted C₂-C₆ alkynyl, unsubstituted 2 to 6 membered heteroalkyl, unsubstituted C₃-C₆ cycloalkyl, or unsubstituted 3 to 6 membered heterocycloalkyl;

In embodiments, R⁵ is independently hydrogen, unsubstituted C₁-C₆ saturated alkyl, unsubstituted C₂-C₆ alkenyl, unsubstituted C₂-C₆ alkynyl, unsubstituted 2 to 6 membered heteroalkyl, unsubstituted C₃-C₆ cycloalkyl, or unsubstituted 3 to 6 membered heterocycloalkyl;

In embodiments, R⁶ is independently hydrogen, unsubstituted C₁-C₆ saturated alkyl, unsubstituted C₂-C₆ alkenyl, unsubstituted C₂-C₆ alkynyl, unsubstituted 2 to 6 membered heteroalkyl, unsubstituted C₃-C₆ cycloalkyl, or unsubstituted 3 to 6 membered heterocycloalkyl;

In embodiments, R⁷ is independently hydrogen, unsubstituted C₁-C₆ saturated alkyl, unsubstituted C₂-C₆ alkenyl, unsubstituted C₂-C₆ alkynyl, unsubstituted 2 to 6 membered heteroalkyl, unsubstituted C₃-C₆ cycloalkyl, or unsubstituted 3 to 6 membered heterocycloalkyl;

In embodiments, R″ is independently hydrogen, unsubstituted C₁-C₆ saturated alkyl, unsubstituted C₂-C₆ alkenyl, unsubstituted C₂-C₆ alkynyl, unsubstituted 2 to 6 membered heteroalkyl, unsubstituted C₃-C₆ cycloalkyl, or unsubstituted 3 to 6 membered heterocycloalkyl;

In embodiments, R⁹ is independently hydrogen, halogen, —CN, —CX⁹₃, —CHX⁹₂, —CH₂X⁹, O—P(O)(OR^{9A})₂, -(unsubstituted saturated alkyl)-O—P(O)(OR^{9A})₂, -(unsubstituted alkoxy)-O—P(O)(OR^{9A})₂, —OR^{9D}, —NR^{9B}R^{9B}, —NR^{9A}R^{9B}, —OC(O)NR^{9A}R^{9B}, —OC(O)R^{9C}, —C(O)R^{9C}, —C(O)—OR^{9C}, —C(O)NR^{9A}R^{9B}, —SOR^{9D}, —SO₂NR^{9A}R^{9B}, —SO₂R^{9D}, —NR^{9A}SO₂R^{9D}, —NR^{9A}C(O)R^{9C}, —NR^{9C}SO₂(u nsubstituted C₁-C₄ alkyl)-NR^{9A}R^{9B}, —N(R^{9C})CO(unsubstituted C₁-C₄ alkyl)-NR^{9A}R^{9B}, —O(unsubstituted C₁-C₄ alkyl)-NR^{9A}R^{9B}, —O(unsubstituted C₁-C₄ alkyl)-OR^{9D}, —O(unsubstituted C₁-C₄ alkyl)-OR^{9B}, —NR^{9C}(unsubstituted C₁-C₄ alkyl)-NR^{9A}R^{9B}, —OCX⁹₃, —OCH₂X⁹, —OCHX⁹₂, —NR^{9C}NR^{9A}R^{9B}, —NR^{9A}R^{9B}, —NHC(O)NR^{9C}NR^{9A}R^{9B}, —NHC(O)NR^{9A}R^{9B}, —N(O)ₘ₉, —NR^{9A}C(O)OR^{9C}, —NR^{9A}OR^{9C}, —SF₅, —N₃, R⁹⁰-substituted or unsubstituted alkyl, R⁹⁰-substituted or unsubstituted heteroalkyl, R⁹⁰-substituted or unsubstituted cycloalkyl, R⁹⁰-substituted or unsubstituted heterocycloalkyl, R⁹⁰-substituted or unsubstituted aryl, or R⁹⁰-substituted or unsubstituted heteroaryl.

In embodiments, R^{9A}, R^{9B}, R^{9C}, and R^{9D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R⁹⁰-substituted or unsubstituted saturated alkyl (e.g., C₁-C₈, C₁-C₆, C₁-C₄, or C₁-C₂), R⁹⁰-substituted or unsubstituted alkenyl (e.g., C₂-C₈, C₂-C₆, C₂-C₄, or C₂), R⁸⁰-substituted or unsubstituted alkynyl (e.g., C₂-C₈, C₂-C₆, C₂-C₄, or C₂), R⁹⁰-substituted or unsubstituted heteroalkyl (e.g., 2 to 8 membered, 2 to 6 membered, 4 to 6 membered, 2 to 3 membered, or 4 to 5 membered), R⁹⁰-substituted or unsubstituted cycloalkyl (e.g., C₃-C₈, C₃-C₆, C₄-C₆, or C₅-C₆), R⁹⁰-substituted or unsubstituted heterocycloalkyl (e.g., 3 to 8 membered, 3 to 6 membered, 4 to 6 membered, 4 to 5 membered, or 5 to 6 membered), R⁹⁰-substituted or unsubstituted aryl (e.g., C₆-C₁₀ or phenyl), or R⁹⁰-substituted or unsubstituted heteroaryl (e.g., 5 to 10 membered, 5 to 9 membered, or 5 to 6 membered).; R^{9A} and R^{9B} substituents bonded to the same nitrogen atom may optionally be joined to form a R⁹⁰-substituted or unsubstituted heterocycloalkyl (e.g., 3 to 8 membered, 3 to 6 membered, 4 to 6 membered, 4 to 5 membered, or 5 to 6 membered) or R⁹⁰-substituted or unsubstituted heteroaryl (e.g., 5 to 10 membered, 5 to 9 membered, or 5 to 6 membered).
R⁹⁰ is halogen, —OR^{90B}, —O—P(O)(OR^{90A})₂, —NR^{90A}R^{90B}, —NR^{90B}R^{90B}, —C(O)—OR^{90B}, —C(O)NR^{90A}R^{90B}, —SOR^{90B}, —SO₂R^{90B}, —SO₂NR^{90A}R^{90B}, —OC(O)R^{90B}, —OC(O)NR^{90A}R^{90B}, —NR^{90A}C(O)R^{90B}, —NR^{90A}SOR^{90B}, —NR^{90A}C(O)OR^{90B}, —C(O)R^{90B}, —CN, —CX⁹⁰₃, —CHX⁹⁰₂, —CH₂X⁹⁰, —NR^{90A}SO₂R^{90B}, —OCX⁹⁰₃, —OCH₂X⁹⁰, —OCHX⁹⁰₂, —SR^{90B}, NHNR^{90A}R^{90B}, —ONR^{90A}R^{90B}, —NHC(O)NHR^{90A}R^{90B}, —NHC(O)NR^{90A}R^{90B}, —N(O)₂, —NR^{90A}OR^{90B}, —SF₅, —N₃, -(unsubstituted saturated alkyl)-O—P(O)(OR^{90A})₂, -(unsubstituted alkoxy)-O— P(O)(OR^{90A})₂, R⁹¹-substituted or unsubstituted saturated alkyl (e.g., C₁-C₈, C₁-C₆, C₁-C₄, or C₁-C₂), R⁹¹-substituted or unsubstituted alkenyl (e.g., C₂-C₈, C₂-C₆, C₂-C₄, or C₂), R⁹¹-substituted or unsubstituted alkynyl (e.g., C₂-C₈, C₂-C₆, C₂-C₄, or C₂), R⁹¹-substituted or unsubstituted heteroalkyl (e.g., 2 to 12 membered, 2 to 6 membered, 4 to 6 membered, 2 to 3 membered, or 4 to 5 membered), R⁹¹-substituted or unsubstituted cycloalkyl (e.g., C₃-C₈, C₃-C₆, C₄-C₆, or C₅-C₆), R⁹¹-substituted or unsubstituted heterocycloalkyl (e.g., 3 to 15 membered, 3 to 6 membered, 4 to 6 membered, 4 to 5 membered, or 5 to 6 membered), R⁹¹-substituted or unsubstituted aryl (e.g., C₆-C₁₀ or phenyl), or R⁹¹-substituted or unsubstituted heteroaryl (e.g., 5 to 10 membered, 5 to 9 membered, or 5 to 6 membered). X⁹⁰ is independently —F, —Cl, —Br, or —I.

In embodiments, R⁹⁰ is halogen, —OR^{90B}, —O—P(O)(OR^{90A})₂, —NR^{90A}R^{90B}, —NR^{90B}R^{90B}, —C(O)—OR^{90B}, —C(O)NR^{90A}R^{90B}, —SOR^{90B}, —SO₂R^{90B}, —SO₂NR^{90A}R^{90B}, —OC(O)R^{90B}, —OC(O)NR^{90A}R^{90B}, —NR^{90A}C(O)R^{90B}, —NR^{90A}SOR^{90B}, —NR^{90A}C(O)OR^{90B}, —C(O)R^{90B}, —CN, —CF₃, —CHF₂, —CH₂F, —NR^{90A}SO₂R^{90B}, —OCF₃, —OCH₂F, —OCHF₂, —SR^{90B}, —NHNR^{90A}R^{90B}, —ONR^{90A}R^{90B}, —NHC(O)NHNR^{90A}R^{90B}, —NHC(O)NR^{90A}R^{90B}, —N(O)₂, —NR^{90A}OR^{90B}, —SF₅, —N₃, R⁹¹-substituted or unsubstituted saturated alkyl (e.g., C₁-C₈, C₁-C₆, C₁-C₄, or C₁-C₂), R⁹¹-substituted or unsubstituted alkenyl (e.g., C₂-C₈, C₂-C₆, C₂-C₄, or C₂), R⁹¹-substituted or unsubstituted alkynyl (e.g., C₂-C₈, C₂-C₆, C₂-C₄, or C₂), R⁹¹-substituted or unsubstituted heteroalkyl (e.g., 2 to 8 membered, 2 to 6 membered, 4 to 6 membered, 2 to 3 membered, or 4 to 5 membered), R⁹¹-substituted or unsubstituted cycloalkyl (e.g., C₃-C₈, C₃-C₆, C₄-C₆, or C₅-C₆), R⁹¹-substituted or unsubstituted heterocycloalkyl (e.g., 3 to 8 membered, 3 to 6 membered, 4 to 6 membered, 4 to 5 membered, or 5 to 6 membered), R⁹¹-substituted or unsubstituted aryl (e.g., C₆-C₁₀ or phenyl), or R⁹¹-substituted or unsubstituted heteroaryl (e.g., 5 to 10 membered, 5 to 9 membered, or 5 to 6 membered).

In embodiments, R⁹⁰ is halogen, —OR^{90B}, —O—P(O)(OR^{90A})₂, —NR^{90A}R^{90B}, —NR^{90B}R^{90B}, —C(O)—OR^{90B}, —C(O)NR^{90A}R^{90B}, —SOR^{90B}, —SO₂R^{90B}, —SO₂NR^{90A}R^{90B}, —OC(O)NR^{90A}R^{90B}, —NR^{90A}SOR^{90B}, —NR^{90A}C(O)OR^{90B}, —CN, —CF₃, —CHF₂, —CH₂F, —NR^{90A}SO₂R^{90B}, —OCF₃, —OCH₂F, —OCHF₂, —SR^{90B}, —NHNR^{90A}R^{90B}, —ONR^{90A}R^{90B}, —NHC(O)NHNR^{90A}R^{90B}, —NHC(O)NR^{90A}R^{90B}, —N(O)₂, —NR^{90A}OR^{90B}, —SF₅, —N₃, R⁹¹-substituted or unsubstituted saturated alkyl (e.g., C₁-C₈, C₁-C₆, C₁-C₄, or C₁-C₂), R⁹¹-substituted or unsubstituted alkenyl (e.g., C₂-C₈, C₂-C₆, C₂-C₄, or C₂), R⁹¹-substituted or unsubstituted alkynyl (e.g., C₂-C₈, C₂-C₆, C₂-C₄, or C₂), R⁹¹-substituted or unsubstituted heteroalkyl (e.g., 2 to 8 membered, 2 to 6 membered, 4 to 6 membered, 2 to 3 membered, or 4 to 5 membered), R⁹¹-substituted or unsubstituted cycloalkyl (e.g., C₃-C₈, C₃-C₆, C₄-C₆, or C₅-C₆), R⁹¹-substituted or unsubstituted heterocycloalkyl (e.g., 3 to 8 membered, 3 to 6 membered, 4 to 6 membered, 4 to 5 membered, or 5 to 6 membered), R⁹¹-substituted or unsubstituted aryl (e.g., C₆-C₁₀ or phenyl), or R⁹¹-substituted or unsubstituted heteroaryl (e.g., 5 to 10 membered, 5 to 9 membered, or 5 to 6 membered); wherein R^{90A} and R^{90B} are independently hydrogen, R⁹¹-substituted or unsubstituted saturated alkyl (e.g., C₁-C₈, C₁-C₆, C₁-C₄, or C₁-C₂), R⁹¹-substituted or unsubstituted alkenyl (e.g., C₂-C₈, C₂-C₆, C₂-C₄, or C₂), R⁹¹-substituted or unsubstituted alkynyl (e.g., C₂-C₈, C₂-C₆, C₂-C₄, or C₂), R⁹¹-substituted or unsubstituted heteroalkyl (e.g., 2 to 8 membered, 2 to 6 membered, 4 to 6 membered, 2 to 3 membered, or 4 to 5 membered), R⁹¹-substituted or unsubstituted cycloalkyl (e.g., C₃-C₈, C₃-C₆, C₄-C₆, or C₅-C₆), R⁹¹-substituted or unsubstituted heterocycloalkyl (e.g., 3 to 8 membered, 3 to 6 membered, 4 to 6 membered, 4 to 5 membered, or 5 to 6 membered), R⁹¹-substituted or unsubstituted aryl (e.g., C₆-C₁₀ or phenyl), or R⁹¹-substituted or unsubstituted heteroaryl (e.g., 5 to 10 membered, 5 to 9 membered, or 5 to 6 membered); R^{90A} and R^{90B} substituents bonded to the same nitrogen atom may optionally be joined to form a R⁹¹-substituted or unsubstituted heterocycloalkyl (e.g., 3 to 8 membered, 3 to 6 membered, 4 to 6 membered, 4 to 5 membered, or 5 to 6 membered) or R⁹¹-substituted or unsubstituted heteroaryl (e.g., 5 to 10 membered, 5 to 9 membered, or 5 to 6 membered); or 2) —OC(O)R^{90B}, —NR^{90A}C(O)R^{90B}, or —C(O)R^{90B}; wherein R^{90A} is independently hydrogen, R⁹¹-substituted or unsubstituted saturated alkyl (e.g., C₁-C₈, C₁-C₆, C₁-C₄, or C₁-C₂), R⁹¹-substituted or unsubstituted alkenyl (e.g., C₂-C₈, C₂-C₆, C₂-C₄, or C₂), R⁹¹-substituted or unsubstituted alkynyl (e.g., C₂-C₈, C₂-C₆, C₂-C₄, or C₂), R⁹¹-substituted or unsubstituted heteroalkyl (e.g., 2 to 8 membered, 2 to 6 membered, 4 to 6 membered, 2 to 3 membered, or 4 to 5 membered), R⁹¹-substituted or unsubstituted cycloalkyl (e.g., C₃-C₈, C₃-C₆, C₄-C₆, or C₅-C₆), R⁹¹-substituted or unsubstituted heterocycloalkyl (e.g., 3 to 8 membered, 3 to 6 membered, 4 to 6 membered, 4 to 5 membered, or 5 to 6 membered), R⁹¹-substituted or unsubstituted aryl (e.g., C₆-C₁₀ or phenyl), or R⁹¹-substituted or unsubstituted heteroaryl (e.g., 5 to 10 membered, 5 to 9 membered, or 5 to 6 membered); R^{90B} is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —COH, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R⁹¹-substituted or unsubstituted saturated alkyl (e.g., C₁-C₈, C₁-C₆, C₁-C₄, or C₁-C₂), R⁹¹-substituted or unsubstituted alkenyl (e.g., C₂-C₈, C₂-C₆, C₂-C₄, or C₂), R⁹¹-substituted or unsubstituted alkynyl (e.g., C₂-C₈, C₂-C₆, C₂-C₄, or C₂), R⁹¹-substituted or unsubstituted heteroalkyl (e.g., 2 to 8 membered, 2 to 6 membered, 4 to 6 membered, 2 to 3 membered, or 4 to 5 membered), R⁹¹-substituted or unsubstituted cycloalkyl (e.g., C₃-C₈, C₃-C₆, C₄-C₆, or C₅-C₆), R⁹¹-substituted or unsubstituted heterocycloalkyl (e.g., 3 to 8 membered, 3 to 6 membered, 4 to 6 membered, 4 to 5 membered, or 5 to 6 membered), R⁹¹-substituted or unsubstituted aryl (e.g., C₆-C₁₀ or phenyl), or R⁹¹-substituted or unsubstituted heteroaryl (e.g., 5 to 10 membered, 5 to 9 membered, or 5 to 6 membered).

In embodiments, R⁹⁰ is halogen, —OR^{90B}, —O—P(O)(OR^{90A})₂, —NR^{90A}R^{90B}, —NR^{90B}R^{90B}, —C(O)—OR^{90B}, —C(O)NR^{90A}R^{90B}, —SOR^{90B}, —SO₂R^{90B}, —SO₂NR^{90A}R^{90B}, —OC(O)R^{90B}, —OC(O)NR^{90A}R^{90B}, —NR^{90A}C(O)R^{90B}, —NR^{90A}SOR^{90B}, —NR^{90A}C(O)OR^{90B}, —C(O)R^{90B}, —CN, —CX⁹⁰₃, —CHX⁹⁰₂, —CH₂X⁹⁰, —NR^{90A}SO₂R^{90B}, —OCX⁹⁰₃, —OCH₂X⁹⁰, —OCHX⁹⁰₂, —SR^{90B}, NHNR^{90A}R^{90B}, —ONR^{90A}R^{90B}, —NHC(O)NHNR^{90A}R^{90B}, —NHC(O)NR^{90A}R^{90B}, —N(O)₂, —NR^{90A}OR^{90B}, —SF₅, —N₃, -(unsubstituted saturated alkyl)-O—P(O)(OR^{90A})₂, -(unsubstituted alkoxy)-O— P(O)(OR^{14A})₂, R⁹¹-substituted or unsubstituted C₁-C₆ saturated alkyl, R⁹¹-substituted or unsubstituted C₂-C₆ alkenyl, R⁹¹-substituted or unsubstituted C₂-C₆ alkynyl, R⁹¹-substituted or unsubstituted 2 to 12 membered heteroalkyl, R⁹¹-substituted or unsubstituted cycloalkyl, R⁹¹-substituted or unsubstituted heterocycloalkyl, R⁹¹-substituted or unsubstituted aryl, or R⁹¹-substituted or unsubstituted heteroaryl. X⁹⁰ is independently —F, —Cl, —Br, or —I.

R^{90A} and R^{90B} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —COH, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R⁹¹-substituted or unsubstituted saturated alkyl (e.g., C₁-C₈, C₁-C₆, C₁-C₄, or C₁-C₂), R⁹¹-substituted or unsubstituted alkenyl (e.g., C₂-C₈, C₂-C₆, C₂-C₄, or C₂), R⁹¹-substituted or unsubstituted alkynyl (e.g., C₂-C₈, C₂-C₆, C₂-C₄, or C₂), R⁹¹-substituted or unsubstituted heteroalkyl (e.g., 2 to 8 membered, 2 to 6 membered, 4 to 6 membered, 2 to 3 membered, or 4 to 5 membered), R⁹¹-substituted or unsubstituted cycloalkyl (e.g., C₃-C₈, C₃-C₆, C₄-C₆, or C₅-C₆), R⁹¹-substituted or unsubstituted heterocycloalkyl (e.g., 3 to 8 membered, 3 to 6 membered, 4 to 6 membered, 4 to 5 membered, or 5 to 6 membered), R⁹¹-substituted or unsubstituted aryl (e.g., C₆-C₁₀ or phenyl), or R⁹¹-substituted or unsubstituted heteroaryl (e.g., 5 to 10 membered, 5 to 9 membered, or 5 to 6 membered); R^{90A} and R^{90B} substituents bonded to the same nitrogen atom may optionally be joined to form a R⁹¹-substituted or unsubstituted heterocycloalkyl (e.g., 3 to 8 membered, 3 to 6 membered, 4 to 6 membered, 4 to 5 membered, or 5 to 6 membered) or R⁹¹-substituted or unsubstituted heteroaryl (e.g., 5 to 10 membered, 5 to 9 membered, or 5 to 6 membered).

In embodiments, R^{90A} and R^{90B} are independently hydrogen, R⁹¹-substituted or unsubstituted saturated alkyl (e.g., C₁-C₈, C₁-C₆, C₁-C₄, or C₁-C₂), R⁹¹-substituted or unsubstituted alkenyl (e.g., C₂-C₈, C₂-C₆, C₂-C₄, or C₂), R⁹¹-substituted or unsubstituted alkynyl (e.g., C₂-C₈, C₂-C₆, C₂-C₄, or C₂), R⁹¹-substituted or unsubstituted heteroalkyl (e.g., 2 to 8 membered, 2 to 6 membered, 4 to 6 membered, 2 to 3 membered, or 4 to 5 membered), R⁹¹-substituted or unsubstituted cycloalkyl (e.g., C₃-C₈, C₃-C₆, C₄-C₆, or C₅-C₆), R⁹¹-substituted or unsubstituted heterocycloalkyl (e.g., 3 to 8 membered, 3 to 6 membered, 4 to 6 membered, 4 to 5 membered, or 5 to 6 membered), R⁹¹-substituted or unsubstituted aryl (e.g., C₆-C₁₀ or phenyl), or R⁹¹-substituted or unsubstituted heteroaryl (e.g., 5 to 10 membered, 5 to 9 membered, or 5 to 6 membered); R^{90A} and R^{90B} substituents bonded to the same nitrogen atom may optionally be joined to form a R⁹¹-substituted or unsubstituted heterocycloalkyl (e.g., 3 to 8 membered, 3 to 6 membered, 4 to 6 membered, 4 to 5 membered, or 5 to 6 membered) or R⁹¹-substituted or unsubstituted heteroaryl (e.g., 5 to 10 membered, 5 to 9 membered, or 5 to 6 membered).
R⁹¹ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, R⁹²-substituted or unsubstituted saturated alkyl (e.g., C₁-C₈, C₁-C₆, C₁-C₄, or C₁-C₂), R⁹²-substituted or unsubstituted alkenyl (e.g., C₂-C₈, C₂-C₆, C₂-C₄, or C₂), R⁹²-substituted or unsubstituted alkynyl (e.g., C₂-C₈, C₂-C₆, C₂-C₄, or C₂), R⁹²-substituted or unsubstituted heteroalkyl (e.g., 2 to 12 membered, 2 to 6 membered, 4 to 6 membered, 2 to 3 membered, or 4 to 5 membered), R⁹²-substituted or unsubstituted cycloalkyl (e.g., C₃-C₈, C₃-C₆, C₄-C₆, or C₅-C₆), R⁹²-substituted or unsubstituted heterocycloalkyl(e.g., 3 to 15 membered, 3 to 6 membered, 4 to 6 membered, 4 to 5 membered, or 5 to 6 membered), R⁹²-substituted or unsubstituted aryl (e.g., C₆-C₁₀ or phenyl), or R⁹²-substituted or unsubstituted heteroaryl (e.g., 5 to 10 membered, 5 to 9 membered, or 5 to 6 membered).

In embodiments, R⁹¹ is independently oxo, halogen, —CF₃, —CHF₂, —CH₂F, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSO₂H, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, —OCH₂F, —SF₅, —N₃, —O—P(O)(OH)₂, R⁹²-substituted or unsubstituted saturated alkyl (e.g., C₁-C₈, C₁-C₆, C₁-C₄, or C₁-C₂), R⁹²-substituted or unsubstituted alkenyl (e.g., C₂-C₈, C₂-C₆, C₂-C₄, or C₂), R⁹²-substituted or unsubstituted alkynyl (e.g., C₂-C₈, C₂-C₆, C₂-C₄, or C₂), R⁹²-substituted or unsubstituted heteroalkyl (e.g., 2 to 8 membered, 2 to 6 membered, 4 to 6 membered, 2 to 3 membered, or 4 to 5 membered), R⁹-substituted or unsubstituted cycloalkyl (e.g., C₃-C₈, C₃-C₆, C₄-C₆, or C₅-C₆), R⁹²-substituted or unsubstituted heterocycloalkyl(e.g., 3 to 8 membered, 3 to 6 membered, 4 to 6 membered, 4 to 5 membered, or 5 to 6 membered), R⁹²-substituted or unsubstituted aryl (e.g., C₆-C₁₀ or phenyl), or R⁹²-substituted or unsubstituted heteroaryl (e.g., 5 to 10 membered, 5 to 9 membered, or 5 to 6 membered).

In embodiments, R⁹¹ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, R⁹²-substituted or unsubstituted C₁-C₄ saturated alkyl, R⁹²-substituted or unsubstituted C₂-C₄ alkenyl, R⁹²-substituted or unsubstituted C₂-C₄ alkynyl, R⁹²-substituted or unsubstituted 2 to 12 membered heteroalkyl, Rn-substituted or unsubstituted C₃-C₆ cycloalkyl, R⁹²-substituted or unsubstituted 3 to 15 membered heterocycloalkyl, Rn-substituted or unsubstituted phenyl, or R⁹²-substituted or unsubstituted 5 to 6 membered heteroaryl.
R⁹² is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, R⁹ is independently oxo, halogen, —CF₃, —CHF₂, —CH₂F, —CN, —OH, —NH₂, —COH, —COOH, —CONH₂, —OCOH, —OCOOH, —OCONH₂, —N₀₂, —SH, —SO₃H, —SO₄H, —SO₂NH₂, —NHSO₂H, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, —OCH₂F, —SF₅, —N₃, —O—P(O)(OH)₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, R¹ is independently R¹⁰-substituted or unsubstituted C₁-C₂₀ saturated alkyl, R¹⁰-substituted or unsubstituted C₂-C₂₀ alkenyl, R¹⁰-substituted or unsubstituted C₂-C₂₀ alkynyl, R¹⁰-substituted or unsubstituted C₃-C₁₀ cycloalkyl, R¹⁰-substituted or unsubstituted 4-10 membered heterocycloalkyl, R¹⁰-substituted or unsubstituted 6-10 membered aryl, or R¹⁰-substituted or unsubstituted 5-10 membered heteroaryl; R¹⁰ is independently halogen, —CN, —CX¹⁰₃, —CHX¹⁰₂, —CH₂X¹⁰, —OR^{10B}, —OC(O)R^{10B}, —OC(O)NR^{10A}R^{10B}, —C(O)—OR^{10B}, —C(O)NR^{10A}R^{10B}, —SOR^{10B}, —SO₂R^{10B}, —SO₂NR^{10A}R^{10B}, —O—P(O)(OR^{10A}), —NR^{10A}R^{10B}, —NR^{10A}C(O)R^{10B}, —NR^{10A}SOR^{10B}, —NR^{10A}C(O)OR^{10B}, —NR^{10A}SO₂R^{10B}, —C(O)R¹⁰, -(unsubstituted alkyl)-O—P(O)OR^{10A})₂, -(unsubstituted alkoxy)-O—P(O)(OR^{10A})₂, —OCX¹⁰₃, —OCH₂X¹⁰, —OCHX¹⁰₂, —SR^{10B}, —NHNR^{10A}R^{10B}, —ONR^{10A}R^{10B}, —NHC(O)NHNR^{10A}R^{10B}, —NHC(O)NR^{10A}R^{10B}, —N(O)₂, —NR^{10A}OR^{10B}, —SF₅, —N₃, R¹¹-substituted or unsubstituted C₁-C₆ saturated alkyl, R¹-substituted or unsubstituted C₂-C₆ alkenyl, R¹¹-substituted or unsubstituted C₂-C₆ alkynyl, R¹¹-substituted or unsubstituted 2 to 8 membered heteroalkyl, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl; X¹⁰ is independently —F, —Cl, —Br, or —I; R^{10A} and R^{10B} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —COH, —COOH, —CONH₂, —OCCl₃, —OCF₃, —OCBr₃, -OCI₃, —OCHCl₂, —OCHBr₂, —OCHI₂, —OCHF₂, —OCH₂Cl, —OCH₂Br, —OCH₂I, —OCH₂F, R¹¹-substituted or unsubstituted C₁-C₁₀ saturated alkyl, R¹¹-substituted or unsubstituted C₂-C₁₀ alkenyl, R¹¹-substituted or unsubstituted C₂-C₁₀ alkynyl, R¹-substituted or unsubstituted 2 to 10 membered heteroalkyl, R¹¹-substituted or unsubstituted cycloalkyl, R¹¹-substituted or unsubstituted heterocycloalkyl, R¹-substituted or unsubstituted aryl, or R¹¹-substituted or unsubstituted heteroaryl; R^{10A} and R^{10B} substituents bonded to the same nitrogen atom may optionally be joined to form a R¹-substituted or unsubstituted heterocycloalkyl (e.g., 3 to 8 membered, 3 to 6 membered, 4 to 6 membered, 4 to 5 membered, or 5 to 6 membered) or R¹-substituted or unsubstituted heteroaryl (e.g., 5 to 10 membered, 5 to 9 membered, or 5 to 6 membered); R¹¹ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, R¹²-substituted or unsubstituted C₁-C₄ saturated alkyl, R¹²-substituted or unsubstituted C₂-C₄ alkenyl, R¹²-substituted or unsubstituted C₂-C₄ alkynyl, R¹²-substituted or unsubstituted 2 to 12 membered heteroalkyl, R¹²-substituted or unsubstituted C₃-C₆ cycloalkyl, R¹²-substituted or unsubstituted 3 to 15 membered heterocycloalkyl, R¹²-substituted or unsubstituted phenyl, or R²-substituted or unsubstituted 5 to 6 membered heteroaryl; R¹² is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, unsubstituted alkyl, unsubstituted heteroalkyl, unsubstituted cycloalkyl, unsubstituted heterocycloalkyl, unsubstituted aryl, or unsubstituted heteroaryl; R² is independently R²⁰-substituted or unsubstituted C₁-C₂₀ saturated alkyl, R²⁰-substituted or unsubstituted C₂-C₂₀ alkenyl, R²⁰-substituted or unsubstituted C₂-C₂₀ alkynyl, R²⁰-substituted or unsubstituted C₃-C₁₀ cycloalkyl, R⁹⁰-substituted or unsubstituted 4-10 membered heterocycloalkyl, R²⁰-substituted or unsubstituted 6-10 membered aryl, or R²⁰-substituted or unsubstituted 5-10 membered heteroaryl; R²⁰ is halogen, —CN, —CX²⁰₃, —CHX²⁰₂, —CH₂X²⁰, —OR^{20B}, —OC(O)R^{20B}, —OC(O)NR^{20A}R^{20B}, —C(O)—OR^{20B}, —C(O)NR^{20A}R^{20B}, —SOR^{20B}, —SO₂R^{20B}, —SO₂NR^{20A}R^{20B}, —O—P(O)(OR^{20A})₂, -(unsubstituted alkyl)-O—P(O)(OR^{20A})₂,-(unsubstituted alkoxy)-O—P(O)(OR^{20A})₂, —NR^{20A}R^{20B}, NR^{20A}C(O)R^{20B}, —NR^{20A}SOR^{20B}, —NR^{20A}C(O)OR^{20B}, —NR^{20A}SO₂R^{20B}, —C(O)R^{20B}, —OCX²⁰₃, —OCH₂X²⁰, —OCHX²⁰₂, —SR^{20B}, —NHNR^{20A}R^{20B}, —ONR^{20A}R^{20B}, —NHC(O)NHNR^{20A}R^{20B}, —NHC(O)NR^{20A}R^{20B}, —N(O)₂, —NR^{20A}OR^{20B}, —SF₅, —N₃, R²¹-substituted or unsubstituted C₁-C₆ saturated alkyl, R²¹-substituted or unsubstituted C₂-C₆ alkenyl, R²¹-substituted or unsubstituted C₂-C₆ alkynyl, R²¹-substituted or unsubstituted 2 to 8 membered heteroalkyl, R²¹-substituted or unsubstituted cycloalkyl, R²¹-substituted or unsubstituted heterocycloalkyl, R²¹-substituted or unsubstituted aryl, or R²¹-substituted or unsubstituted heteroaryl; X²⁰ is independently —F, —Cl, —Br, or —I; R^{20A} and R^{20B} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —COH, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R²¹-substituted or unsubstituted C₁-C₁₀ saturated alkyl, R²¹-substituted or unsubstituted C₂-C₁₀ alkenyl, R²¹-substituted or unsubstituted C₂-C₁₀ alkynyl, R²¹-substituted or unsubstituted 2 to 10 membered heteroalkyl, R²¹-substituted or unsubstituted cycloalkyl, R²¹-substituted or unsubstituted heterocycloalkyl, R²¹-substituted or unsubstituted aryl, or R²¹-substituted or unsubstituted heteroaryl; R^{20A} and R^{20B} substituents bonded to the same nitrogen atom may optionally be joined to form a R²¹-substituted or unsubstituted heterocycloalkyl or R²¹-substituted or unsubstituted heteroaryl; R²¹ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, R²²-substituted or unsubstituted C₁-C₄ saturated alkyl, R²²-substituted or unsubstituted C₂-C₄ alkenyl, R²²-substituted or unsubstituted C₂-C₄ alkynyl, R²²-substituted or unsubstituted 2 to 12 membered heteroalkyl, R²²-substituted or unsubstituted C₃-C₆ cycloalkyl, R²²-substituted or unsubstituted 3 to 15 membered heterocycloalkyl, R²²-substituted or unsubstituted phenyl, or R²²-substituted or unsubstituted 5 to 6 membered heteroaryl; R²² is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, unsubstituted alkyl, unsubstituted heteroalkyl, unsubstituted cycloalkyl, unsubstituted heterocycloalkyl, unsubstituted aryl, or unsubstituted heteroaryl; R³ is independently halogen, —OR^{3D}, —CN, —CX³₃, —CHX³₂, —CH₂X³, —C(O)NR^{3A}R^{3B}, —C(O)—OR^{3C}, —O—P(O)(OH)₂, —NR^{3A}R^{3B}, —C(O)R^{3C}, —NR^{3A}C(O)R^{3C}, —NR^{3A}SO₂R^{3D}, —NR^{3C}SO₂(unsubstituted C₁-C₆ alkyl)-NR^{3A}R^{3B}, —NR^{3C}C(O)(unsubstituted C₁-C₆alkyl)-NR^{3A}R^{3B}, (unsubstituted C₂-C₆alkyl)-NR^{3E}-(unsubstituted C₂-C₆ alkyl), R³¹-substituted or unsubstituted alkyl, R³¹-substituted or unsubstituted heteroalkyl, or R³¹-substituted or unsubstituted cycloalkyl; R^{3A}, R^{3B}, R^{3C}, R^{3D}, R^{3F}, and R^{3G} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R³¹-substituted or unsubstituted alkyl, R³¹-substituted or unsubstituted heteroalkyl, R³¹-substituted or unsubstituted cycloalkyl, R³¹-substituted or unsubstituted heterocycloalkyl, R³¹-substituted or unsubstituted aryl, or R³¹-substituted or unsubstituted heteroaryl; R^{3A} and R^{3B} substituents bonded to the same nitrogen atom may optionally be joined to form a R³¹-substituted or unsubstituted heterocycloalkyl or R³¹-substituted or unsubstituted heteroaryl; R^{3F} and R^{3G} substituents bonded to the same nitrogen atom may optionally be joined to form a R³¹-substituted or unsubstituted heterocycloalkyl or R³¹-substituted or unsubstituted heteroaryl; R^{3E} is independently hydrogen, —C(O)R^{3F}, —C(O)—OR^{3F}, —SOR^{3F}, —SO₂R^{3F}, —C(O)NR^{3F}R^{3G}, —SO₂NR^{3F}R^{3G}; —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, R³¹-substituted or unsubstituted alkyl, R³¹-substituted or unsubstituted heteroalkyl, R³¹-substituted or unsubstituted cycloalkyl, R³¹-substituted or unsubstituted heterocycloalkyl, R³¹-substituted or unsubstituted aryl, or R³¹-substituted or unsubstituted heteroaryl; R^{3A} and R^{3B} substituents bonded to the same nitrogen atom may optionally be joined to form a R³¹-substituted or unsubstituted heterocycloalkyl or R³¹-substituted or unsubstituted heteroaryl; R³¹ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, R³²-substituted or unsubstituted C₁-C₄ saturated alkyl, R³²-substituted or unsubstituted C₂-C₄ alkenyl, R³²-substituted or unsubstituted C₂-C₄ alkynyl, R³²-substituted or unsubstituted 2 to 12 membered heteroalkyl, R³²-substituted or unsubstituted C₃-C₆ cycloalkyl, R³²-substituted or unsubstituted 3 to 15 membered heterocycloalkyl, R³²-substituted or unsubstituted phenyl, or R³²-substituted or unsubstituted 5 to 6 membered heteroaryl; R³² is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, R³³-substituted or unsubstituted C₁-C₄ saturated alkyl, R³³-substituted or unsubstituted C₂-C₄ alkenyl, R³³-substituted or unsubstituted C₂-C₄ alkynyl, R³³-substituted or unsubstituted 2 to 12 membered heteroalkyl, R³³-substituted or unsubstituted C₃-C₆ cycloalkyl, R³³-substituted or unsubstituted 3 to 15 membered heterocycloalkyl, R³³-substituted or unsubstituted phenyl, or R³³-substituted or unsubstituted 5 to 6 membered heteroaryl; R³³ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, unsubstituted alkyl, unsubstituted heteroalkyl, unsubstituted cycloalkyl, unsubstituted heterocycloalkyl, unsubstituted aryl, or unsubstituted heteroaryl; R⁴ is independently halogen, —OR, —CN, —CX⁴₃, —CHX⁴₂, —CH₂X⁴, —C(O)NR^{4A}R^{4B}, —C(O)—OR^{c}, —O—P(O)(OH)₂, —NR^{4A}R^{4B}, —C(O)R^{4C}, —NR^{4A}C(O)R^{4C}, —N^{4A}SO₂R^{4D}, —NR^{4C}SO₂(unsubstituted C₁-C₆ alkyl)-NR^{4A}R^{4B}, —NR^{4C}C(O)(unsubstituted C₁-C₆alkyl)-NR^{4A}R^{4B}, (unsubstituted C₂-C₆alkyl)-NR^{4E}-(unsubstituted C₂-C₆ alkyl), R⁴¹-substituted or unsubstituted alkyl, R⁴¹-substituted or unsubstituted heteroalkyl, or R⁴¹-substituted or unsubstituted cycloalkyl; R^{4A}, R^{4B}, R^{4C}, R^{4D}, R^{4F}, and R^{4G} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R⁴¹-substituted or unsubstituted alkyl, R⁴¹-substituted or unsubstituted heteroalkyl, R⁴¹-substituted or unsubstituted cycloalkyl, R⁴¹-substituted or unsubstituted heterocycloalkyl, R⁴¹-substituted or unsubstituted aryl, or R⁴¹-substituted or unsubstituted heteroaryl; R^{4A} and R^{4B} substituents bonded to the same nitrogen atom may optionally be joined to form a R⁴¹-substituted or unsubstituted heterocycloalkyl or R⁴¹-substituted or unsubstituted heteroaryl; R^{4F} and R^{4G} substituents bonded to the same nitrogen atom may optionally be joined to form a R⁴¹-substituted or unsubstituted heterocycloalkyl or R⁴¹-substituted or unsubstituted heteroaryl; R^{4E} is independently hydrogen, —C(O)R^{4F}, —C(O)—OR^{4F}, —SOR^{4F}, —SO₂R^{4F}, —C(O)NR^{4F}R^{4G}, —SO₂NR^{4F}R^{4G}; —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R⁴¹-substituted or unsubstituted alkyl, R⁴¹-substituted or unsubstituted heteroalkyl, R⁴¹-substituted or unsubstituted cycloalkyl, R⁴¹-substituted or unsubstituted heterocycloalkyl, R⁴¹-substituted or unsubstituted aryl, or R⁴¹-substituted or unsubstituted heteroaryl; R⁴¹ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, R⁴²-substituted or unsubstituted C₁-C₄ saturated alkyl, R⁴²-substituted or unsubstituted C₂-C₄ alkenyl, R⁴²-substituted or unsubstituted C₂-C₄ alkynyl, R⁴²-substituted or unsubstituted 2 to 12 membered heteroalkyl, R⁴²-substituted or unsubstituted C₃-C₆ cycloalkyl, R⁴²-substituted or unsubstituted 3 to 15 membered heterocycloalkyl, R⁴²-substituted or unsubstituted phenyl, or R⁴²-substituted or unsubstituted 5 to 6 membered heteroaryl; R⁴² is independently oxo, halogen, —CCl₃, —CBr₃, —CF₃, —CI₃, —CHCl₂, —CHBr₂, —CHF₂, —CHI₂, —CH₂C₁, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂C₁, —OCH₂Br, —OCH₂I, —OCH₂F, —SF₅, —N₃, —O—P(O)(OH)₂, R⁴³-substituted or unsubstituted C₁-C₄ saturated alkyl, R⁴³-substituted or unsubstituted C₂-C₄ alkenyl, R⁴³-substituted or unsubstituted C₂-C₄ alkynyl, R⁴³-substituted or unsubstituted 2 to 12 membered heteroalkyl, R⁴³-substituted or unsubstituted C₃-C₆ cycloalkyl, R⁴³-substituted or unsubstituted 3 to 15 membered heterocycloalkyl, R⁴³-substituted or unsubstituted phenyl, or R⁴³-substituted or unsubstituted 5 to 6 membered heteroaryl; R⁴³ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, unsubstituted alkyl, unsubstituted heteroalkyl, unsubstituted cycloalkyl, unsubstituted heterocycloalkyl, unsubstituted aryl, or unsubstituted heteroaryl; L is R^{L}-substituted or unsubstituted C₁-C₁₀ saturated alkyl, R^{L}-substituted or unsubstituted C₂-C₁₀ alkenyl, R^{L}-substituted or unsubstituted C₂-C₁₀ alkynyl, R^{L}-substituted or unsubstituted C₂-C₆ alkyl-O—C₂-C₆ alkyl, R^{L}-substituted or unsubstituted C₂ alkyl-O—C₂ alkyl, R^{L}-substituted or unsubstituted C₂ alkyl-O—C₂ alkyl-O—C₂ alkyl, R^{L}-substituted or unsubstituted C₂-C₆ alkyl-N(R^{L4})-C₂-C₆ alkyl, R^{L}-substituted or unsubstituted C₃-C₆ cycloalkyl, R^{L}-substituted or unsubstituted phenyl, R^{L}-substituted or unsubstituted 4-6 membered heterocycloalkyl, R^{L}-substituted or unsubstituted 5-6 membered heteroaryl, R^{L}-substituted or unsubstituted C₁-C₄alkyl-(C₃-C₆ cycloalkyl)-C₁-C₄alkyl, R^{L}-substituted or unsubstituted C₁-C₄ alkyl-phenyl-C₁-C₄ alkyl, R^{L}-substituted or unsubstituted C₁-C₄ alkyl-(4-6 membered heterocycloalkyl)-C₁-C₄ alkyl, or R^{L}-substituted or unsubstituted C₁-C₄ alkyl-(5-6 membered heteroaryl)-C₁-C₄ alkyl; R^{L} is halogen, —OR^{LB}, —O—P(O)(OR^{LA})₂, -(unsubstituted alkyl)-O—P(O)(OR^{LA})₂, -(unsubstituted alkoxy)-O—P(O)(OR^{LA})₂, —NR^{LA}R^{LB}, —OC(O)R^{LB}, —C(O)—OR^{LB}, —SOR^{LB}, —SO₂R^{LB}, —C(O)NR^{LA}R^{LB}, —SO₂NR^{LA}R^{LB}, —OC(O)NR^{LA}R^{LB}, —NR^{LA}C(O)R^{LB}, —NR^{LA}SOR^{LB}, —NR^{LA}C(O)OR^{LB}, —NR^{LA}SO₂R^{LB}, —R^{LA} SO₂R^{LB}, —CN, —CX^{L}₃, —CHX^{L}₂, —CH₂X^{L}, —C(O)R^{LB}, —OCX^{L}₃, —OCH₂X^{L}, —OCHX^{L}₂, —SR^{LB}, —NHNR^{LA}R^{LB}, —ONR^{LA}R^{LB}, —NHC(O)NHNR^{LA}R^{LB}, —NHC(O)NR^{LA}R^{LB}, —N(O)₂, —NR^{LA}OR^{LB}, —SF₅, —N₃, R^{L6}-substituted or unsubstituted C₁-C₆ saturated alkyl, R^{L6}-substituted or unsubstituted C₂-C₆ alkenyl, R^{L6}-substituted or unsubstituted C₂-C₆ alkynyl, R^{L6}-substituted or unsubstituted 2 to 12 membered heteroalkyl, R^{L6}-substituted or unsubstituted cycloalkyl, R^{L6}-substituted or unsubstituted heterocycloalkyl, R^{L6}-substituted or unsubstituted aryl, or R^{L}-substituted or unsubstituted heteroaryl; X^{L} is independently —F, —Cl, —Br, or —I; R^{LA} and R^{LB} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —COH, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R^{L6}-substituted or unsubstituted saturated alkyl, R^{L6}-substituted or unsubstituted alkenyl, R^{L6}-substituted or unsubstituted alkynyl, R^{L6}-substituted or unsubstituted heteroalkyl, R^{L6}-substituted or unsubstituted cycloalkyl, R^{L6}-substituted or unsubstituted heterocycloalkyl, R^{L6}-substituted or unsubstituted aryl, or R^{L}-substituted or unsubstituted heteroaryl; R^{L6} is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, R^{L7}-substituted or unsubstituted C₁-C₄ saturated alkyl, R^{L7}-substituted or unsubstituted C₂-C₄ alkenyl, R^{L7}-substituted or unsubstituted C₂-C₄ alkynyl, R^{L7}-substituted or unsubstituted 2 to 12 membered heteroalkyl, R^{L7}-substituted or unsubstituted C₃-C₆ cycloalkyl, R^{L7}-substituted or unsubstituted 3 to 15 membered heterocycloalkyl, R^{L7}-substituted or unsubstituted phenyl, or R^{L7}-substituted or unsubstituted 5 to 6 membered heteroaryl; R^{L7} is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, unsubstituted alkyl, unsubstituted heteroalkyl, unsubstituted cycloalkyl, unsubstituted heterocycloalkyl, unsubstituted aryl, or unsubstituted heteroaryl; R^{L4} is independently hydrogen, COR^{L4A}, CO₂H, CO₂R^{L4A}, SOR^{L4A}, SO₂R^{L4A}, CONH₂, CONR^{L4A}R^{L4B}, SO₂NH₂, or SO₂NR^{L4A}R^{L4B}; R^{L6}-substituted or unsubstituted alkyl, R^{L6}-substituted or unsubstituted heteroalkyl, R^{L6}-substituted or unsubstituted cycloalkyl, R^{L6}-substituted or unsubstituted heterocycloalkyl, R^{L6}-substituted or unsubstituted aryl, or R^{L6}-substituted or unsubstituted heteroaryl; R^{L4A} and R^{L4B} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R^{L6}-substituted or unsubstituted alkyl, R^{L6}-substituted or unsubstituted heteroalkyl, R^{L6}-substituted or unsubstituted cycloalkyl, R^{L6}-substituted or unsubstituted heterocycloalkyl, R^{L6}-substituted or unsubstituted aryl, or R^{L6}-substituted or unsubstituted heteroaryl; R^{L4A} and R^{L4B} substituents bonded to the same nitrogen atom may optionally be joined to form a R^{L6}-substituted or unsubstituted heterocycloalkyl or R^{L6}-substituted or unsubstituted heteroaryl; L¹ is independently ═NC(O)—, —C(O)N(R^{L1})—, or —N(R^{L1})C(O)—; R^{L1} is independently hydrogen, unsubstituted C₁-C₆ saturated alkyl, unsubstituted C₂-C₆ alkenyl, unsubstituted C₂-C₆ alkynyl, unsubstituted 2 to 6 membered heteroalkyl, unsubstituted C₃-C₆ cycloalkyl, or unsubstituted 3 to 6 membered heterocycloalkyl; L² is independently=NC(O)—, —C(O)N(R^{L2})—, or —N(R^{L2})C(O)—; R^{L2} is independently hydrogen, unsubstituted C₁-C₆ saturated alkyl, unsubstituted C₂-C₆ alkenyl, unsubstituted C₂-C₆ alkynyl, unsubstituted 2 to 6 membered heteroalkyl, unsubstituted C₃-C₆ cycloalkyl, or unsubstituted 3 to 6 membered heterocycloalkyl; A⁵ is independently O, S, N, NR⁵, or CR⁵; R⁵ is independently hydrogen, unsubstituted C₁-C₆ saturated alkyl, unsubstituted C₂-C₆ alkenyl, unsubstituted C₂-C₆ alkynyl, unsubstituted 2 to 6 membered heteroalkyl, unsubstituted C₃-C₆ cycloalkyl, or unsubstituted 3 to 6 membered heterocycloalkyl; A⁶ is independently O, S, N, NR, or CR⁶; R⁶ is independently hydrogen, unsubstituted C₁-C₆ saturated alkyl, unsubstituted C₂-C₆ alkenyl, unsubstituted C₂-C₆ alkynyl, unsubstituted 2 to 6 membered heteroalkyl, unsubstituted C₃-C₆ cycloalkyl, or unsubstituted 3 to 6 membered heterocycloalkyl; A⁷ is independently N or CR⁷; R⁷ is independently hydrogen, unsubstituted C₁-C₆ saturated alkyl, unsubstituted C₂-C₆ alkenyl, unsubstituted C₂-C₆ alkynyl, unsubstituted 2 to 6 membered heteroalkyl, unsubstituted C₃-C₆ cycloalkyl, or unsubstituted 3 to 6 membered heterocycloalkyl; A⁸ is independently N or CR⁸; R⁸ is independently hydrogen, unsubstituted C₁-C₆ saturated alkyl, unsubstituted C₂-C₆ alkenyl, unsubstituted C₂-C₆ alkynyl, unsubstituted 2 to 6 membered heteroalkyl, unsubstituted C₃-C₆ cycloalkyl, or unsubstituted 3 to 6 membered heterocycloalkyl; A⁹ is independently N or CR⁹; R⁹ is independently hydrogen, halogen, —CN, —CX⁹₃, —CHX⁹₂, —CH₂X⁹, O—P(O)(OR^{9A})₂, -(unsubstituted saturated alkyl)-O—P(O)(OR^{9A})₂, -(unsubstituted alkoxy)-O—P(O)(OR^{9A})₂, —OR^{9D}, —NR^{9B}R^{9B}, —NR^{9A}R^{9B}, —OC(O)NR^{9A}R^{9B}, —OC(O)R^{9C}, —C(O)R^{9C}, —C(O)—OR^{9C}, —C(O)NR^{9A}R^{9B}, —SOR^{9D}, —SO₂NR^{9A}R^{9B}, —SO₂R^{9D}, —NR^{9A}SO₂R^{9D}, —NR^{9A}C(O)R^{9C}, —NR^{9C}SO₂(u nsubstituted C₁-C₄ alkyl)-NR^{9A}R^{9B}, —N(R^{9C})CO(unsubstituted C₁-C₄ alkyl)-NR^{9A}R^{9B}, —O(unsubstituted C₁-C₄ alkyl)-NR^{9A}R^{9B}, —O(unsubstituted C₁-C₄ alkyl)-OR^{9D}, —O(unsubstituted C₁-C₄ alkyl)-OR^{9B}, —NR^{9C}(unsubstituted C₁-C₄ alkyl)-NR^{9A}R^{9B}, —OCX⁹₃, —OCH₂X⁹, —OCHX⁹₂, —NR^{9C}NR^{9A}R^{9B}, —NR^{9A}R^{9B}, —NHC(O)NR^{9C}NR^{9A}R^{9B}, —NHC(O)NR^{9A}R^{9B}, —N(O)ₘ₉, —NR^{9A}C(O)OR^{9C}, —NR^{9A}OR^{9C}, —SF₅, —N₃, R⁹⁰-substituted or unsubstituted alkyl, R⁹⁰-substituted or unsubstituted heteroalkyl, R⁹⁰-substituted or unsubstituted cycloalkyl, R⁹⁰-substituted or unsubstituted heterocycloalkyl, R⁹⁰-substituted or unsubstituted aryl, or R⁹⁰-substituted or unsubstituted heteroaryl; R^{9A}, R^{9B}, R^{9C}, and R^{9D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R⁹⁰-substituted or unsubstituted saturated alkyl (e.g., C₁-C₈, C₁-C₆, C₁-C₄, or C₁-C₂), R⁹⁰-substituted or unsubstituted alkenyl (e.g., C₂-C₈, C₂-C₆, C₂-C₄, or C₂), R⁹⁰-substituted or unsubstituted alkynyl (e.g., C₂-C₈, C₂-C₆, C₂-C₄, or C₂), R⁹⁰-substituted or unsubstituted heteroalkyl (e.g., 2 to 8 membered, 2 to 6 membered, 4 to 6 membered, 2 to 3 membered, or 4 to 5 membered), R⁹⁰-substituted or unsubstituted cycloalkyl (e.g., C₃-C₈, C₃-C₆, C₄-C₆, or C₅-C₆), R⁹⁰-substituted or unsubstituted heterocycloalkyl (e.g., 3 to 8 membered, 3 to 6 membered, 4 to 6 membered, 4 to 5 membered, or 5 to 6 membered), R⁹⁰-substituted or unsubstituted aryl (e.g., C₆-C₁₀ or phenyl), or R⁹⁰-substituted or unsubstituted heteroaryl (e.g., 5 to 10 membered, 5 to 9 membered, or 5 to 6 membered).; R^{9A} and R^{9B} substituents bonded to the same nitrogen atom may optionally be joined to form a R⁹⁰-substituted or unsubstituted heterocycloalkyl (e.g., 3 to 8 membered, 3 to 6 membered, 4 to 6 membered, 4 to 5 membered, or 5 to 6 membered) or R⁹⁰-substituted or unsubstituted heteroaryl (e.g., 5 to 10 membered, 5 to 9 membered, or 5 to 6 membered); R⁹⁰ is halogen, —OR^{90B}, —O—P(O)(OR^{90A})₂, —NR^{90A}R^{90B}, —NR^{90B}R^{90B}, —C(O)—OR^{90B}, —C(O)NR^{90A}R^{90B}, —SOR^{90B}, —SO₂R^{90B}, —SO₂NR^{90A}R^{90B}, —OC(O)R^{90B}, —OC(O)NR^{90A}R^{90B}, —NR⁹% C(O)R^{90B}, —NR^{90A}SOR^{90B}, —NR^{90A}C(O)OR^{90B}, —C(O)R^{90B}, —CN, —CX⁹₃, —CHX⁹⁰₂, —CH₂X⁹⁰, —NR^{90A}SO₂R^{90B}, —OCX⁹⁰₃, —OCH₂X⁹⁰, —OCHX⁹⁰₂, —SR^{90B}, NHNR^{90A}R^{90B}, —ONR^{90A}R^{90B}, —NHC(O)NHNR^{90A}R^{90B}, —NHC(O)NR^{90A}R^{90B}, —N(O)₂, —NR^{90A}OR^{90B}, —SF₅, —N₃, -(unsubstituted saturated alkyl)-O—P(O)(OR^{90A})₂, -(unsubstituted alkoxy)-O— P(O)(OR^{90A})₂, R⁹¹-substituted or unsubstituted C₁-C₆ saturated alkyl, R⁹¹-substituted or unsubstituted C₂-C₆ alkenyl, R⁹¹-substituted or unsubstituted C₂-C₆ alkynyl, R⁹¹-substituted or unsubstituted 2 to 12 membered heteroalkyl, R⁹¹-substituted or unsubstituted cycloalkyl, R⁹¹-substituted or unsubstituted heterocycloalkyl, R⁹¹-substituted or unsubstituted aryl, or R⁹¹-substituted or unsubstituted heteroaryl; X⁹⁰ is independently —F, —Cl, —Br, or —I; R^{90A} and R^{90B} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —COH, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R⁹¹-substituted or unsubstituted saturated alkyl, R⁹¹-substituted or unsubstituted alkenyl, R⁹¹-substituted or unsubstituted alkynyl, R⁹¹-substituted or unsubstituted heteroalkyl, R⁹¹-substituted or unsubstituted cycloalkyl, R⁹¹-substituted or unsubstituted heterocycloalkyl, R⁹¹-substituted or unsubstituted aryl, or R⁹¹-substituted or unsubstituted heteroaryl; R^{90A} and R^{90B} substituents bonded to the same nitrogen atom may optionally be joined to form a R⁹¹-substituted or unsubstituted heterocycloalkyl or R⁹¹-substituted or unsubstituted heteroaryl; R⁹¹ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, R⁹²-substituted or unsubstituted C₁-C₄ saturated alkyl, R⁹²-substituted or unsubstituted C₂-C₄ alkenyl, R⁹²-substituted or unsubstituted C₂-C₄ alkynyl, R⁹²-substituted or unsubstituted 2 to 12 membered heteroalkyl, Rn-substituted or unsubstituted C₃-C₆ cycloalkyl, R⁹²-substituted or unsubstituted 3 to 15 membered heterocycloalkyl, Rn-substituted or unsubstituted phenyl, or R⁹²-substituted or unsubstituted 5 to 6 membered heteroaryl; R⁹ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, unsubstituted alkyl, unsubstituted heteroalkyl, unsubstituted cycloalkyl, unsubstituted heterocycloalkyl, unsubstituted aryl, or unsubstituted heteroaryl.

In certain embodiments, provided herein are compounds of formula (Ia):

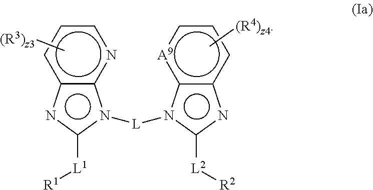

In embodiments, R¹ is independently R¹⁰-substituted or unsubstituted C₁-C₂₀ saturated alkyl, R¹⁰-substituted or unsubstituted C₂-C₂₀ alkenyl, R¹⁰-substituted or unsubstituted C₂-C₂₀ alkynyl, R¹⁰-substituted or unsubstituted C₃-C₁₀ cycloalkyl, R¹⁰-substituted or unsubstituted phenyl, R¹⁰-substituted or unsubstituted 6-10 membered aryl, R¹⁰-substituted or unsubstituted 4-10 membered heterocycloalkyl, or R¹⁰-substituted or unsubstituted 5-10 membered heteroaryl. In embodiments, said R¹⁰-substituted C₁-C₂₀ saturated alkyl, R¹⁰-substituted C₂-C₂₀ alkenyl, R¹⁰-substituted C₂-C₂₀ alkynyl, R¹⁰-substituted C₃-C₁₀ cycloalkyl, R¹⁰-substituted phenyl, R¹⁰-substituted 6-10 membered aryl, R¹⁰-substituted 4-10 membered heterocycloalkyl, and R¹⁰-substituted 5-10 membered heteroaryl is substituted with 1 to 4 R¹⁰ substituents.

In embodiments, each R¹⁰ substituent is independently halogen, —CN, —CF₃, —OR^{10B}, —OC(O)R^{10B}, —OC(O)NR^{10A}R^{10B}, —C(O)—OR^{10B}, —C(O)NR^{10A}R^{10B}, -s OR^{10B}, —SO₂R^{10B}, —SO₂NR^{10A}R^{10B}, —O—P(O)(OR^{10A})₂, —NR^{10A}R^{10B}, —NR^{10A}C(O)R^{10B}, —NR^{10A}SOR^{10B}, —NR^{10A}C(O)OR^{10B}, —NR^{10A}SO₂R^{10B}, —C(O)R^{10B}, unsubstituted C₁-C₆ saturated alkyl, unsubstituted C₂-C₆ alkenyl, unsubstituted C₂-C₆ alkynyl, halo-substituted (C₁-C₄ alkyl), hydroxy-substituted (C₁-C₄ alkyl), (unsubstituted C₁-C₄ alkyl)-O—P(O)(OH)₂, (unsubstituted C₁-C₄ alkyl)-O—P(O)(OR^{10A})₂, halo-substituted (C₁-C₄ alkoxy), unsubstituted C₁-C₆ alkylthio, hydroxy-substituted (C₂-C₄ alkoxy), (unsubstituted C₂-C₄ alkoxy)-O—P(O)(OH)₂, (unsubstituted C₂-C₄ alkoxy)-O—P(O)(OR^{10A})₂, unsubstituted C₁-C₄ alkoxy(unsubstituted C₁-C₄ alkoxy).

In embodiments, R^{10A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy.

In embodiments, R^{10B} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R¹¹-substituted or unsubstituted phenyl, R¹¹-substituted or unsubstituted heteroaryl, R¹¹-substituted or unsubstituted heterocycloalkyl, or R¹¹-substituted or unsubstituted heteroaryl; wherein said R¹¹-substituted or unsubstituted phenyl, R¹¹-substituted or unsubstituted heteroaryl, R¹¹-substituted or unsubstituted heterocycloalkyl, and R¹¹-substituted or unsubstituted heteroaryl are optionally substituted with one or more R¹¹ substituents.

In embodiments, R¹¹ is independently halogen, CN, CF₃, unsubstituted C₁-C₄ saturated alkyl, halo-substituted (saturated C₁-C₄ alkyl), unsubstituted C₂-C₄ alkenyl, unsubstituted C₂-C₄ alkynyl, or unsubstituted C₃-C₆ cycloalkyl.

In embodiments, R² is independently R²⁰-substituted or unsubstituted C₁-C₂₀ saturated alkyl, R²⁰-substituted or unsubstituted C₂-C₂₀ alkenyl, R²⁰-substituted or unsubstituted C₂-C₂₀ alkynyl, R²⁰-substituted or unsubstituted C₃-C₁₀ cycloalkyl, R²⁰-substituted or unsubstituted phenyl, R²⁰-substituted or unsubstituted 6-10 membered aryl, R²⁰-substituted or unsubstituted 4-10 membered heterocycloalkyl, or R²⁰-substituted or unsubstituted 5-10 membered heteroaryl. In embodiments, said R²⁰-substituted C₁-C₂₀ saturated alkyl, R²⁰-substituted C₂-C₂₀ alkenyl, R²⁰-substituted C₂-C₂₀ alkynyl, R²⁰-substituted C₃-C₁₀ cycloalkyl, R²⁰-substituted phenyl, R²⁰-substituted 6-10 membered aryl, R²⁰-substituted 4-10 membered heterocycloalkyl, and R²⁰-substituted 5-10 membered heteroaryl is substituted with 1 to 4 R²⁰ substituents.

In embodiments, each R²⁰ substituent is independently halogen, —CN, —CF₃, —OR^{20B}, —OC(O)R^{20B}, —OC(O)NR^{20A}R^{20B}, —C(O)—OR^{20B}, —C(O)NR^{20A}R^{20B}, —SOR^{20B}, —SO₂R^{20B}, —SO₂NR^{20A}R^{20B}, —O—P(O)(OR^{20A})₂, —N^{20A}R^{20B}, —NR^{20A}C(O)R^{20B}, —NR^{20A}SOR^{20B}, —NR^{20A}C(O)OR^{20B}, —NR^{20A}SO₂R^{20B}, —C(O)R^{20B}, unsubstituted C₁-C₆ saturated alkyl, unsubstituted C₂-C₆ alkenyl, unsubstituted C₂-C₆ alkynyl, halo-substituted (C₁-C₄ alkyl), hydroxy-substituted (C₁-C₄ alkyl), (unsubstituted C₁-C₄ alkyl)-O—P(O)(OH)₂, (unsubstituted C₁-C₄ alkyl)-O—P(O)OR^{20A})₂, halo-substituted (C₁-C₄ alkoxy), unsubstituted C₁-C₆ alkylthio, hydroxy-substituted (C₂-C₄ alkoxy), (unsubstituted C₂-C₄ alkoxy)-O—P(O)(OH)₂, (unsubstituted C₂-C₄ alkoxy)-O—P(O)(OR^{20A})₂, unsubstituted C₁-C₄ alkoxy(unsubstituted C₁-C₄ alkoxy).

In embodiments, R^{20A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy.

In embodiments, R^{20B} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R²¹-substituted or unsubstituted phenyl, R²¹-substituted or unsubstituted heteroaryl, R²¹-substituted or unsubstituted heterocycloalkyl, or R²¹-substituted or unsubstituted heteroaryl; wherein said R²¹-substituted or unsubstituted phenyl, R²¹-substituted or unsubstituted heteroaryl, R²¹-substituted or unsubstituted heterocycloalkyl, and R²¹-substituted or unsubstituted heteroaryl are optionally substituted with one or more R²¹ substituents.

In embodiments, R²¹ is independently halogen, CN, CF₃, unsubstituted C₁-C₄ saturated alkyl, halo-substituted (saturated C₁-C₄ alkyl), unsubstituted C₂-C₄ alkenyl, unsubstituted C₂-C₄ alkynyl, or unsubstituted C₃-C₆ cycloalkyl.

In embodiments, R³ is independently halogen, —CN, —CF₃, —C(O)NH₂, —C(O)OR^{3′}, —O—P(O)(OH)₂, —NR^{3A}R^{3B}, —C(O)R^{3C}, —NR^{3A}C(O)R^{3C}, —OR^{3D}, —NR^{3A}SO₂R^{3D}, unsubstituted C₂-C₆ saturated alkyl-NR^{3E}— unsubstituted C₂-C₆ saturated alkyl, N(R^{3H})SO₂(unsubstituted C₁-C₆ saturated alkyl)-N(R^{3H}(R³), N(R^{3H})CO(unsubstituted C₁-C₆ saturated alkyl)-N(R^{3H}(R^{3I}), unsubstituted C₁-C₆ saturated alkyl, unsubstituted C₂-C₆ alkenyl, unsubstituted C₂-C₆ alkynyl, unsubstituted C₁-C₆ saturated alkylthio, or unsubstituted C₁-C₆ cycloalkyl.

In embodiments, R^{3A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy.

In embodiments, R^{3B}, R^{3C}, and R^{ID} are independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R³¹-substituted or unsubstituted phenyl, R³¹-substituted or unsubstituted heteroaryl, R³¹-substituted or unsubstituted heterocycloalkyl, or R³-substituted or unsubstituted heteroaryl; wherein said R³¹-substituted or unsubstituted phenyl, R³¹-substituted or unsubstituted heteroaryl, R³¹-substituted or unsubstituted heterocycloalkyl, and R³¹-substituted or unsubstituted heteroaryl are optionally substituted with one or more R³¹ substituents.

In embodiments, R³¹ is independently halogen, CN, CF₃, unsubstituted C₁-C₄ saturated alkyl, halo-substituted (saturated C₁-C₄ alkyl), unsubstituted C₂-C₄ alkenyl, unsubstituted C₂-C₄ alkynyl, or unsubstituted C₃-C₆ cycloalkyl.

In embodiments, R³¹ is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, substituted or unsubstituted aryl, substituted or unsubstituted heteroaryl, or substituted or unsubstituted heterocycloalkyl, wherein said substituted aryl, substituted heteroaryl, and substituted heterocyclic groups are independently substituted with one or more halogen, CN, CF₃, or unsubstituted C₁-C₄ saturated alkyl.

In embodiments, R^{3E} is independently hydrogen, —C(O)R^{3F}, —C(O)OR^{3F}, —SOR^{3F}, —SO₂R^{3F}, —C(O)NR^{3F}R^{3G}, or —SO₂NR^{3F}R^{3G}.

In embodiments, R^{3G} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy.

In embodiments, R^{3F} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R³¹-substituted or unsubstituted phenyl, R³¹-substituted or unsubstituted heteroaryl, R³¹-substituted or unsubstituted heterocycloalkyl, or R³¹-substituted or unsubstituted heteroaryl; wherein said R³¹-substituted or unsubstituted phenyl, R³¹-substituted or unsubstituted heteroaryl, R³¹-substituted or unsubstituted heterocycloalkyl, and R³¹-substituted or unsubstituted heteroaryl are optionally substituted with one or more R³¹ substituents.

In embodiments, R^{3H} and R^{3I} are independently hydrogen, substituted or unsubstituted C₁-C₆ saturated alkyl, wherein said substituted saturated alkyl is independently substituted with one or more halogen, OH, amine, CN, CF₃ or unsubstituted C₁-C₄ saturated alkyl; or R^{3H} and R^{3I}, taken together with the atom or atoms through which they are connected to form a substituted or unsubstituted 5-6 membered heterocycloalkyl or substituted or unsubstituted 5-6 membered heteroaryl, wherein the substituted 5-6 membered heterocycloalkyl or substituted 5-6 membered heteroaryl is independently substituted with one or more halogen, OH, amine, CN, CF₃, or unsubstituted C₁-C₄ saturated alkyl, wherein said amine substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy.

In embodiments, R⁴ is independently halogen, —CN, —CF₃, —C(O)NH₂, —C(O)OR^{4J}, —O—P(O)(OH)₂, —N^{4A}R^{4B}, —C(O)R^{4C}, —NR^{4A}C(O)R^{4C}, —OR, —N^{4A}SO₂R^{4D}, unsubstituted C₂-C₆ saturated alkyl-NR^{4E}— unsubstituted C₂-C₆ saturated alkyl, N(R^{4H})SO₂(unsubstituted C₁-C₆ saturated alkyl)-N(R^{4H})(R^{4I}), N(R^{4H})CO(unsubstituted C₁-C₆ saturated alkyl)-N(R^{4H}) R^{4I}), unsubstituted C₁-C₆ saturated alkyl, unsubstituted C₂-C₆ alkenyl, unsubstituted C₂-C₆ alkynyl, unsubstituted C₁-C₆ saturated alkylthio, or unsubstituted C₁-C₆ cycloalkyl.

In embodiments, R^{4A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy.

In embodiments, R^{4B}, R^{4C}, and R^{4D} are independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R⁴¹-substituted or unsubstituted phenyl, R⁴¹-substituted or unsubstituted heteroaryl, R⁴¹-substituted or unsubstituted heterocycloalkyl, or R⁴¹-substituted or unsubstituted heteroaryl; wherein said R⁴¹-substituted or unsubstituted phenyl, R⁴¹-substituted or unsubstituted heteroaryl, R⁴¹-substituted or unsubstituted heterocycloalkyl, and R⁴¹-substituted or unsubstituted heteroaryl are optionally substituted with one or more R⁴¹ substituents.

In embodiments, R⁴¹ is independently halogen, CN, CF₃, unsubstituted C₁-C₄ saturated alkyl, halo-substituted (saturated C₁-C₄ alkyl), unsubstituted C₂-C₄ alkenyl, unsubstituted C₂-C₄ alkynyl, or unsubstituted C₃-C₆ cycloalkyl.

In embodiments, R⁴¹ is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, substituted or unsubstituted aryl, substituted or unsubstituted heteroaryl, or substituted or unsubstituted heterocycloalkyl, wherein said substituted aryl, substituted heteroaryl, and substituted heterocyclic groups are independently substituted with one or more halogen, CN, CF₃, or unsubstituted C₁-C₄ saturated alkyl.

In embodiments, R^{4E} is independently hydrogen, —C(O)R^{4F}, —C(O)OR^{4F}, —SOR^{4F}, —SO₂R^{4F}, —C(O)NR^{4F}R^{4G}, or —SO₂NR^{4F}R^{4G}.

In embodiments, R^{4G} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy.

In embodiments, R^{4F} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R⁴¹-substituted or unsubstituted phenyl, R⁴¹-substituted or unsubstituted heteroaryl, R⁴¹-substituted or unsubstituted heterocycloalkyl, or R⁴¹-substituted or unsubstituted heteroaryl; wherein said R⁴¹-substituted or unsubstituted phenyl, R⁴¹-substituted or unsubstituted heteroaryl, R⁴¹-substituted or unsubstituted heterocycloalkyl, and R⁴¹-substituted or unsubstituted heteroaryl are optionally substituted with one or more R⁴ substituents.

In embodiments, R^{4H} and R^{4I} are independently hydrogen, substituted or unsubstituted C₁-C₆ saturated alkyl, wherein said substituted saturated alkyl is independently substituted with one or more halogen, OH, amine, CN, CF₃, or unsubstituted C₁-C₄ saturated alkyl; or R^{4H} and R^{4I}, taken together with the atom or atoms through which they are connected to form a substituted or unsubstituted 5-6 membered heterocycloalkyl or substituted or unsubstituted 5-6 membered heteroaryl, wherein the substituted 5-6 membered heterocycloalkyl or substituted 5-6 membered heteroaryl is independently substituted with one or more halogen, OH, amine, CN, CF₃, or unsubstituted C₁-C₄ saturated alkyl, wherein said amine substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy.

In embodiments, L is halo-substituted (C₁-C₁₀ saturated alkylene), R^{L}-substituted or unsubstituted C₁-C₁₀ alkylene, R^{L}-substituted or unsubstituted C₂-C₁₀ alkenylene, R^{L}-substituted or unsubstituted C₂-C₁₀ alkynylene, R^{L}-substituted or unsubstituted C₂-C₆ saturated alkylene-O—C₂-C₆ saturated alkylene, R^{L}-substituted or unsubstituted C₂ saturated alkylene-O—C₂ saturated alkylene, R^{L}-substituted or unsubstituted C₂ saturated alkylene-O—C₂ saturated alkylene-O—C₂ saturated alkylene, R^{L}-substituted or unsubstituted C₂-C₆ saturated alkylene-NR^{L4}—C₂-C₆ saturated alkylene, R^{L}-substituted or unsubstituted C₃-C₆ cycloalkylene, R^{L}-substituted or unsubstituted phenylene, R^{L}-substituted or unsubstituted 4-6 membered heterocycloalkylene, R^{L}-substituted or unsubstituted 5-6 membered heteroarylene, R^{L}-substituted or unsubstituted C₁-C₄ saturated alkylene-(C₃-C₆ cycloalkylene)-C₁-C₄ saturated alkylene, R^{L}-substituted or unsubstituted C₁-C₄ saturated alkylene-phenylene-C₁-C₄ saturated alkylene, R^{L}-substituted or unsubstituted C₁-C₄ saturated alkylene-(4-6 membered heterocycloalkylene)-C₁-C₄ saturated alkylene, or R^{L}-substituted or unsubstituted C₁-C₄ saturated alkylene-(5-6 membered heteroarylene)-C₁-C₄ saturated alkylene; In embodiments, said R^{L}-substituted C₁-C₁₀alkyl, R^{L}-substituted C₂-C₁₀ alkenyl, R^{L}-substituted C₂-C₁₀ alkynyl, R^{L}-substituted C₂-C₆ saturated alkyl-O—C₂-C₆ saturated alkyl, R^{L}-substituted C₂ saturated alkyl-O—C₂ saturated alkyl, R^{L}-substituted C₂ saturated alkyl-O—C₂ saturated alkyl-O—C₂ saturated alkyl, R^{L}-substituted C₂-C₆ saturated alkyl-NR^{L}—C₂-C₆ saturated alkyl, R^{L}-substituted C₃-C₆ cycloalkyl, R^{L}-substituted phenyl, R^{L}-substituted 4-6 membered heterocycloalkyl, R^{L}-substituted 5-6 membered heteroaryl, R^{L}-substituted C₁-C₄ saturated alkyl-(C₃-C₆ cycloalkyl)-C₁-C₄ saturated alkyl, R^{L}-substituted C₁-C₄ saturated alkyl-phenyl-C₁-C₄ saturated alkyl, R^{L}-substituted C₁-C₄ saturated alkyl-(4-6 membered heterocycloalkyl)-C₁-C₄ saturated alkyl, and R^{L}-substituted C₁-C₄ saturated alkyl-(5-6 membered heteroaryl)-C₁-C₄ saturated alkyl are independently substituted with 1 or 2 R^{L}; R^{L} is independently halogen, —OR^{LB}, —O—P(O)(OR^{LA})₂, —NR^{LA}R^{LB}, —OC(O)R^{LB}, —C(O)—OR^{LB}, —SOR^{LB}, —SO₂R^{LB}, —C(O)NR^{LA}R^{LB}, —SO₂NR^{L A}R^{LB}, —OC(O)NR^{LA}R^{LB}, —NR^{LA}C(O)R^{LB}, —NR^{LA}SOR^{LB}, —NR^{LA}C(O)OR^{LB}, —NR^{LA}SO₂R^{LB}, —R^{LA}S O₂R^{LB}, halo-substituted (saturated C₁-C₄alkyl), (unsubstituted C₁-C₄ saturated alkyl)(unsubstituted C₁-C₄ saturated alkyl)amino, unsubstituted C₁-C₄ saturated alkyl, hydroxy-substituted (C₁-C₄ saturated alkyl), (unsubstituted C₁-C₄ saturated alkyl)-O—P(O)(OH)₂, (unsubstituted C₁-C₄ saturated alkyl)-O—P(O)OR^{LA})₂, halo-substituted (C₁-C₄ alkoxy), unsubstituted C₁-C₄ alkoxy, unsubstituted C₁-C₆ saturated alkylthio, hydroxy-substituted (C₂-C₄ alkoxy), (unsubstituted C₂-C₄ alkoxy)-O—P(O)(OH)₂, (unsubstituted C₂-C₄alkoxy)-O—P(O)(OR^{LA})₂, unsubstituted C₁-C₄ alkoxy(unsubstituted C₁-C₄ alkoxy), COR^{LA}, or CON(R^{LA})(R^{LC}).

In embodiments, R^{LA} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy.

In embodiments, R^{LB} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R^{L6}-substituted or unsubstituted phenyl, R^{L6}-substituted or unsubstituted heteroaryl, R^{L6}-substituted or unsubstituted heterocycloalkyl, or R^{L6}-substituted or unsubstituted heteroaryl; wherein said R^{L6}-substituted or unsubstituted phenyl, R^{L6}-substituted or unsubstituted heteroaryl, R^{L6}-substituted or unsubstituted heterocycloalkyl, and R^{L6}-substituted or unsubstituted heteroaryl are optionally substituted with one or more R^{L6} substituents.

In embodiments, R^{L6} is independently halogen, CN, CF₃, unsubstituted C₁-C₄ saturated alkyl, halo-substituted (saturated C₁-C₄ alkyl), unsubstituted C₂-C₄ alkenyl, unsubstituted C₂-C₄ alkynyl, or unsubstituted C₃-C₆ cycloalkyl.

R^{LC} is independently hydrogen, substituted or unsubstituted saturated alkyl (e.g., C₁-C₈, C₁-C₆, C₁-C₄, or C₁-C₂), wherein the substituted saturated alkyl is independently substituted with one or more halogen, OH, amine, CN, CF₃, or unsubstituted saturated alkyl (e.g., C₁-C₈, C₁-C₆, C₁-C₄, or C₁-C₂).

In embodiments, R^{LC} is independently hydrogen, substituted or unsubstituted C₁-C₆ saturated alkyl, wherein the substituted saturated alkyl is independently substituted with one or more halogen, OH, amine, CN, CF₃, or unsubstituted C₁-C₄ saturated alkyl.

In embodiments, R^{L4} is independently hydrogen, COR^{L4A}, CO₂H, CO₂R^{L4A}, SOR^{L4A}, SO₂R^{L4A}, CONH₂, CONR^{L4A}R^{L4B}, SO₂NH₂, or SO₂NR^{L4A}R^{L4B}.

In embodiments, R^{L4B} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy.

In embodiments, R^{L4A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R^{L6}-substituted or unsubstituted phenyl, R^{L6}-substituted or unsubstituted heterocycloalkyl, or R^{L6}-substituted or unsubstituted heteroaryl; wherein said R^{L6}-substituted or unsubstituted phenyl, R^{L6}-substituted or unsubstituted heterocycloalkyl, and R^{L6}-substituted or unsubstituted heteroaryl are optionally substituted with one or more R^{L6} substituents.

L¹ is independently=NC(O)—, —C(O)N(R^{L1})—, or —N(R^{L1})C(O)—;

In embodiments, R^{L1} is independently hydrogen, unsubstituted C₁-C₆ saturated alkyl, unsubstituted C₂-C₆ alkenyl, unsubstituted C₂-C₆ alkynyl, or unsubstituted C₃-C₆ cycloalkyl.

L² is independently=NC(O)—, —C(O)N(R^{L2})—, or —N(R^{L2})C(O)—;

In embodiments, R^{L2} is independently hydrogen, unsubstituted C₁-C₆ saturated alkyl, unsubstituted C₂-C₆ alkenyl, unsubstituted C₂-C₆ alkynyl, or unsubstituted C₃-C₆ cycloalkyl;

A⁹ is independently N or CR⁹;

In embodiments, R⁹ is independently hydrogen, halogen, —CN, —CF₃, O—P(O)(OR^{9A})₂, —OR^{9D}, —NR^{9B}R^{9B}, —NR^{9A}R^{9B}, —OC(O)NR^{9A}R^{9B}, —OC(O)R^{9C}, —C(O)R^{9C}, —C(O)—OR^{9J}, —C(O)NR^{9A}R^{9B}, —SOR^{9D}, —SO₂NR^{9A}R^{9B}, —SO₂R⁹¹, —NR^{9A}SO₂R^{9D}, —NR^{9A}C(O)R^{9C}, N(R^{9H})SO₂(unsubstituted C₁-C₄ saturated alkyl)-N(R^{9I})(R^{9H}), N(R^{9H})CO(unsubstituted C₁-C₄ saturated alkyl)-N(R^{9I}(R^{9H}), O(unsubstituted C₁-C₄ saturated alkyl)-N(R^{9I})(R^{9H}), O(unsubstituted C₁-C₄ saturated alkyl)-OR^{9B}, NR^{9H}(unsubstituted C₁-C₄ saturated alkyl)-N(R^{9I})(R^{9H}), halo-substituted (C₁-C₁₀ saturated alkyl), halo-substituted (C₁-C₁₀ alkoxy), R⁹⁰-substituted or unsubstituted (C₁-C₁₀ saturated alkyl)oxy, R⁹⁰-substituted or unsubstituted (C₁-C₁₀ saturated alkyl)amino, R⁹⁰-substituted or unsubstituted (C₁-C₆ saturated alkyl)(C₁-C₄ saturated alkyl)amino, R⁹⁰-substituted or unsubstituted phenyl, R⁹⁰-substituted or unsubstituted 5-6 membered heterocycloalkyl, or R⁹⁰-substituted or unsubstituted 5-6 membered heteroaryl.

In embodiments, R^{9A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy.

In embodiments, R^{9B}, R^{9C}, and R^{9D} are independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R⁹¹-substituted or unsubstituted phenyl, R⁹¹-substituted or unsubstituted heteroaryl, R⁹¹-substituted or unsubstituted heterocycloalkyl, or R⁹¹-substituted or unsubstituted heteroaryl; wherein said R⁹¹-substituted or unsubstituted phenyl, R⁹¹-substituted or unsubstituted heteroaryl, R⁹¹-substituted or unsubstituted heterocycloalkyl, and R⁹¹-substituted or unsubstituted heteroaryl are optionally substituted with one or more R⁹¹ substituents.

In embodiments, R⁹¹ is independently halogen, CN, CF₃, unsubstituted C₁-C₄ saturated alkyl, halo-substituted (saturated C₁-C₄ alkyl), unsubstituted C₂-C₄ alkenyl, unsubstituted C₂-C₄ alkynyl, or unsubstituted C₃-C₆ cycloalkyl.

In embodiments, R^{9J} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, substituted or unsubstituted aryl, substituted or unsubstituted heteroaryl, or substituted or unsubstituted heterocycloalkyl, wherein said substituted aryl, substituted heteroaryl, and substituted heterocyclic groups are independently substituted with one or more halogen, CN, CF₃, or unsubstituted C₁-C₄ saturated alkyl.

In embodiments, R^{9H} and R^{9I} are independently hydrogen, substituted or unsubstituted C₁-C₆ saturated alkyl, wherein said substituted saturated alkyl is independently substituted with one or more halogen, OH, amine, CN, CF₃, or unsubstituted C₁-C₄ saturated alkyl; or R^{9H} and R^{9I}, taken together with the atom or atoms through which they are connected to form a substituted or unsubstituted 5-6 membered heterocycloalkyl or substituted or unsubstituted 5-6 membered heteroaryl, wherein the substituted 5-6 membered heterocycloalkyl or substituted 5-6 membered heteroaryl is independently substituted with one or more halogen, OH, amine, CN, CF₃, or unsubstituted C₁-C₄ saturated alkyl, wherein said amine substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy;

In embodiments, said R⁹⁰-substituted (C₁-C₁₀ saturated alkyl)oxy, R⁹⁰-substituted (C₁-C₁₀ saturated alkyl)amino, R⁹⁰-substituted (C₁-C₆ saturated alkyl)(C₁-C₄ saturated alkyl)amino, R⁹⁰-substituted phenyl, R⁹⁰-substituted 5-6 membered heterocycloalkyl, or R⁹⁰-substituted 5-6 membered heteroaryl is substituted with 1-4 independent R⁹⁰.

In embodiments, R⁹⁰ is halogen, —OR^{90B}, —O—P(O)(OR^{90A})₂, —NR^{90A}R^{90B}, —NR^{90B}R^{90B}, —C(O)—OR^{90B}, —C(O)NR^{90A}R^{90B}, —SOR^{90B}, —SO₂R^{90B}, —SO₂NR^{90A}R^{90B}, —OC(O)R^{90B}, —OC(O)NR^{90A}R^{90B}, —NR^{90A}C(O)R^{90B}, —NR^{90A}SOR^{90B}, —NR^{90A}C(O)OR^{90B}, —C(O)R^{90B}, —SO₂R^{90J}, —C(O)—OR^{90J}, —C(O)NR^{90A}R^{90H}, —C(O)R^{90A}, (unsubstituted C₁-C₄ saturated alkyl)(unsubstituted C₁-C₄ saturated alkyl)amino, unsubstituted C₁-C₄ saturated alkyl, halo-substituted (C₁-C₄ saturated alkyl), hydroxy-substituted (C₁-C₄ saturated alkyl), (unsubstituted C₁-C₄ saturated alkyl)-O—P(O)(OH)₂, (unsubstituted C₁-C₄ saturated alkyl)-O—P(O)(OR^{90A})₂, halo-substituted (C₁-C₄ alkoxy), unsubstituted C₁-C₄ alkoxy, hydroxy-substituted (C₂-C₄ alkoxy), (unsubstituted C₂-C₄ alkoxy)-O—P(O)(OH)₂, (unsubstituted C₂-C₄alkoxy)-O—P(O)(OR^{90A})₂, or unsubstituted C₁-C₄ alkoxy-(unsubstituted C₁-C₄ alkoxy).

In embodiments, R^{90A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy.

In embodiments, R^{90B} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R⁹¹-substituted or unsubstituted phenyl, R⁹¹-substituted or unsubstituted heteroaryl, R⁹¹-substituted or unsubstituted heterocycloalkyl, or R⁹¹-substituted or unsubstituted heteroaryl; wherein said R⁹¹-substituted or unsubstituted phenyl, R⁹¹-substituted or unsubstituted heteroaryl, R⁹¹-substituted or unsubstituted heterocycloalkyl, and R⁹¹-substituted or unsubstituted heteroaryl are optionally substituted with one or more R⁹¹ substituents.

In embodiments, R⁹¹ is independently halogen, CN, CF₃, unsubstituted C₁-C₄ saturated alkyl, halo-substituted (saturated C₁-C₄ alkyl), unsubstituted C₂-C₄ alkenyl, unsubstituted C₂-C₄ alkynyl, or unsubstituted C₃-C₆ cycloalkyl.

In embodiments, R^{90J} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, substituted or unsubstituted aryl, substituted or unsubstituted heteroaryl, or substituted or unsubstituted heterocycloalkyl, wherein said substituted aryl, substituted heteroaryl, and substituted heterocyclic groups are independently substituted with one or more halogen, CN, CF₃, or unsubstituted C₁-C₄ saturated alkyl.

In embodiments, R^{90H} is independently hydrogen, substituted or unsubstituted C₁-C₆ saturated alkyl, wherein said substituted saturated alkyl is independently substituted with one or more halogen, OH, amine, CN, CF₃, or unsubstituted C₁-C₄ saturated alkyl, wherein said amine substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy; or a tautomer thereof; or a salt thereof (e.g., a pharmaceutically acceptable salt).

In embodiments, the compound is a compound having formula (Ib)

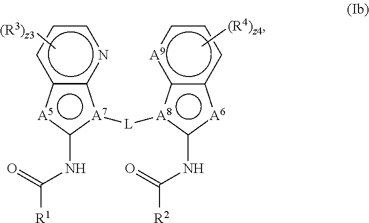

or a tautomer thereof; or a salt thereof (e.g., a pharmaceutically acceptable salt). R¹, R², R³, z3, R⁴, z4, L, A⁵, A⁶, A⁷, As, and A⁹ are as described herein, including in embodiments.

In embodiments, the compound is a compound having formula (Ic)

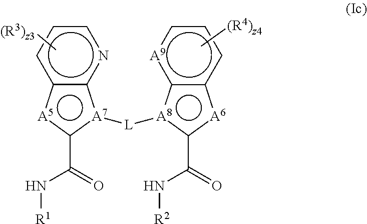

or a tautomer thereof; or a salt thereof (e.g., a pharmaceutically acceptable salt). R¹, R², R³, z3, R⁴, z4, L, A⁵, A⁶, A⁷, A⁸, and A⁹ are as described herein, including in embodiments.

In embodiments, the compound is a compound having formula (Id)

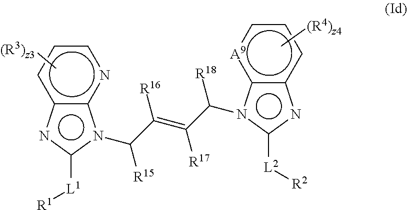

or a tautomer thereof, or a salt thereof (e.g., a pharmaceutically acceptable salt). R¹, L¹, R², L², R³, z3, R⁴ z4, R¹⁵, R¹⁶, R¹⁷, R¹⁸, and A⁹ are as described herein, including in embodiments.

R¹⁵, R¹⁶, R¹⁷, and R¹⁸ are independently hydrogen, halogen, —CN, —CF₃, —CONH₂, —COOH, —COR^{19B}, —CO₂R^{19J}, unsubstituted C₁-C₆ saturated alkyl, unsubstituted C₂-C₆ alkenyl, unsubstituted C₂-C₆ alkynyl, or unsubstituted C₃-C₆ cycloalkyl.

R^{19B} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R^{29B}-substituted or unsubstituted phenyl, R^{29B}-substituted or unsubstituted heteroaryl, R^{29B}-substituted or unsubstituted heterocycloalkyl, or R^{29B}-substituted or unsubstituted heteroaryl; wherein said R^{29B}-substituted or unsubstituted phenyl, R^{29B}-substituted or unsubstituted heteroaryl, R^{29B}-substituted or unsubstituted heterocycloalkyl, and R^{29B}-substituted or unsubstituted heteroaryl are optionally substituted with one or more R^{29B}.

R^{29B} is independently halogen, CN, CF₃, unsubstituted C₁-C₄ saturated alkyl, halo-substituted (saturated C₁-C₄ alkyl), unsubstituted C₂-C₄ alkenyl, unsubstituted C₂-C₄ alkynyl, or unsubstituted C₃-C₆ cycloalkyl.

R^{19J} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, substituted or unsubstituted aryl, substituted or unsubstituted heteroaryl, or substituted or unsubstituted heterocycloalkyl, wherein said substituted aryl, substituted heteroaryl, and substituted heterocyclic groups are independently substituted with one or more halogen, —CN, —CF₃, or unsubstituted C₁-C₄ saturated alkyl.

In embodiments, the compound is:

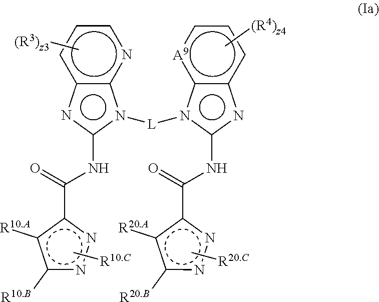

or a tautomer thereof, or a salt thereof (e.g., a pharmaceutically acceptable salt). R^{10.A}, R^{10.B}, and R^{10.C} are independently hydrogen or any value of R¹⁰ as described herein, including in embodiments. R^{20.A}, R^{20.B}, and R^{20.C} are independently hydrogen or any value of R²⁰ as described herein, including in embodiments.

In embodiments, R^{10.A} is independently hydrogen, halogen, or unsubstituted C₁-C₄ saturated alkyl.

In embodiments, R^{10.B} is independently hydrogen, R¹¹-substituted or unsubstituted C₁-C₆ saturated alkyl, R¹¹-substituted or unsubstituted C₂-C₆ alkenyl, R¹¹-substituted or unsubstituted C₂-C₆ alkynyl, R¹¹-substituted or unsubstituted 2 to 7 membered alkoxy, R¹¹-substituted or unsubstituted C₁-C₆ alkylamino, R¹¹-substituted or unsubstituted C₃-C₆ cycloalkyl, R¹¹-substituted or unsubstituted aryl, R¹¹-substituted or unsubstituted heterocycloalkyl, or R″-substituted or unsubstituted heteroaryl.

In embodiments, R^{10.C} is independently hydrogen, R¹¹-substituted or unsubstituted C₁-C₆ saturated alkyl, R¹¹-substituted or unsubstituted C₂-C₆ alkenyl, R¹¹-substituted or unsubstituted C₂-C₆ alkynyl, or R¹-substituted or unsubstituted C₃-C₆ cycloalkyl.

In embodiments, R¹¹ is independently halogen, —CN, —CF₃, —OR^{11B}, —NR^{11A}R^{11B}, —C(O)—OR^{11B}, —C(O)NR^{11A}R^{11B}, —SO₂NR^{11A}R^{11B}, or —OC(O)NR^{11A}R^{11B}.

R^{11A} is independently hydrogen, unsubstituted saturated alkyl (e.g., C₁-C₉, C₁-C₆, C₁-C₄, or C₁-C₂), unsubstituted alkenyl (e.g., C₂-C₈, C₂-C₆, C₂-C₄, or C₂), unsubstituted alkynyl (e.g., C₂-C₈, C₂-C₆, C₂-C₄, or C₂), hydroxy-substituted saturated alkyl (e.g., C₁-C₈, C₁-C₆, C₁-C₄, or C₁-C₂), amino-substituted alkyl (e.g., C₁-C₈, C₁-C₆, C₁-C₄, or C₁-C₂), or thiol-substituted saturated alkyl (e.g., C₁-C₈, C₁-C₆, C₁-C₄, or C₁-C₂), wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy.

In embodiments, R^{11A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy.

R^{11B}, is independently hydrogen, unsubstituted saturated alkyl (e.g., C₁-C₈, C₁-C₆, C₁-C₄, or C₁-C₂), unsubstituted alkenyl (e.g., C₂-C₈, C₂-C₆, C₂-C₄, or C₂), unsubstituted alkynyl (e.g., C₂-C₈, C₂-C₆, C₂-C₄, or C₂), hydroxy-substituted saturated alkyl (e.g., C₁-C₈, C₁-C₆, C₁-C₄, or C₁-C₂), R¹²-substituted or unsubstituted aryl (e.g., C₆-C₁₀ or phenyl), R¹²-substituted or unsubstituted heteroaryl (e.g., 5 to 10 membered, 5 to 9 membered, or 5 to 6 membered), or R¹²-substituted or unsubstituted heterocycloalkyl (e.g., 3 to 8 membered, 3 to 6 membered, 4 to 6 membered, 4 to 5 membered, or 5 to 6 membered); wherein said R¹²-substituted aryl, R¹²-substituted heteroaryl, and R¹²-substituted heterocycloalkyl, are substituted with one or more R¹² substituents.

In embodiments, R^{11B}, is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R¹²-substituted or unsubstituted phenyl, R¹²-substituted or unsubstituted heteroaryl, or R¹²-substituted or unsubstituted heterocycloalkyl; wherein said R¹²-substituted phenyl, R¹²-substituted heteroaryl, and R¹²-substituted heterocycloalkyl are substituted with one or more R¹² substituents.

In embodiments, R¹² is independently halogen, CN, CF₃, unsubstituted C₁-C₄ saturated alkyl, halo-substituted (saturated C₁-C₄ alkyl), unsubstituted C₂-C₄ alkenyl, unsubstituted C₂-C₄ alkynyl, or unsubstituted C₃-C₆ cycloalkyl.

R^{20.A} is independently hydrogen, halogen, or unsubstituted saturated alkyl (e.g., C₁-C₈, C₁-C₆, C₁-C₄, or C₁-C₂).

In embodiments, R^{2A} is independently hydrogen, halogen, or unsubstituted C₁-C₄ saturated alkyl.

R^{20.B} is independently hydrogen, R²¹-substituted or unsubstituted saturated alkyl (e.g., C₁-C₈, C₁-C₆, C₁-C₄, or C₁-C₂), R²¹-substituted or unsubstituted alkenyl (e.g., C₂-C₈, C₂-C₆, C₂-C₄, or C₂), R²¹-substituted or unsubstituted alkynyl (e.g., C₂-C₈, C₂-C₆, C₂-C₄, or C₂), R²¹-substituted or unsubstituted alkoxy (e.g., C₁-C₈, C₁-C₆, C₁-C₄, or C₁-C₂), R²¹-substituted or unsubstituted alkylamino (e.g., C₁-C₈, C₁-C₆, C₁-C₄, or C₁-C₂), R²¹-substituted or unsubstituted cycloalkyl (e.g., C₃-C₈, C₃-C₆, C₄-C₆, or C₅-C₆), R²¹-substituted or unsubstituted aryl (e.g., C₆-C₁₀ or phenyl), R²¹-substituted or unsubstituted heterocycloalkyl (e.g., 3 to 8 membered, 3 to 6 membered, 4 to 6 membered, 4 to 5 membered, or 5 to 6 membered), or R²¹-substituted or unsubstituted heteroaryl (e.g., 5 to 10 membered, 5 to 9 membered, or 5 to 6 membered).

In embodiments, R^{20.B} is independently hydrogen, R²¹-substituted or unsubstituted C₁-C₆ saturated alkyl, R²¹-substituted or unsubstituted C₂-C₆ alkenyl, R²¹-substituted or unsubstituted C₂-C₆ alkynyl, R²¹-substituted or unsubstituted 2 to 7 membered alkoxy, R²¹-substituted or unsubstituted C₁-C₆ alkylamino, R²¹-substituted or unsubstituted C₃-C₆ cycloalkyl, R²¹-substituted or unsubstituted aryl, R²¹-substituted or unsubstituted heterocycloalkyl, or R²¹-substituted or unsubstituted heteroaryl.

R^{20.C} is independently hydrogen, R²¹-substituted or unsubstituted saturated alkyl (e.g., C₁-C₈, C₁-C₆, C₁-C₄, or C₁-C₂), R²¹-substituted or unsubstituted alkenyl (e.g., C₂-C₈, C₂-C₆, C₂-C₄, or C₂), R²¹-substituted or unsubstituted alkynyl (e.g., C₂-C₈, C₂-C₆, C₂-C₄, or C₂), or R²¹-substituted or unsubstituted cycloalkyl (e.g., C₃-C₈, C₃-C₆, C₄-C₆, or C₅-C₆).

In embodiments, R^{20.C} is independently hydrogen, R²¹-substituted or unsubstituted C₁-C₆ saturated alkyl, R²¹-substituted or unsubstituted C₂-C₆ alkenyl, R²¹-substituted or unsubstituted C₂-C₆ alkynyl, or R²¹-substituted or unsubstituted C₃-C₆ cycloalkyl.

In embodiments, R²¹ is independently halogen, —CN, —CF₃, —OR^{21B}, —NR^{21A}R^{21B}, —C(O)—OR²¹B, —C(O)NR^{21A}R^{21B}, —SO₂NR^{21A}R^{21B}, or —OC(O)NR^{21A}R^{21B}.

In embodiments, R^{21A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy.

In embodiments, R^{21B} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R²²-substituted or unsubstituted phenyl, R²²-substituted or unsubstituted heteroaryl, R²²-substituted or unsubstituted heterocycloalkyl, or R²²-substituted or unsubstituted heteroaryl; wherein said R²²-substituted or unsubstituted phenyl, R²²-substituted or unsubstituted heteroaryl, R²²-substituted or unsubstituted heterocycloalkyl, and R²²-substituted or unsubstituted heteroaryl are optionally substituted with one or more R²² substituents.

In embodiments, R²² is independently halogen, CN, CF₃, unsubstituted C₁-C₄ saturated alkyl, halo-substituted (saturated C₁-C₄ alkyl), unsubstituted C₂-C₄ alkenyl, unsubstituted C₂-C₄ alkynyl, or unsubstituted C₃-C₆ cycloalkyl.

In embodiments, R³ is independently halogen, —OH, —CN, —CF₃, —C(O)NH₂, —C(O)OR^{3J}, —O—P(O)(OH)₂, —NR^{3A}R^{3B}, —C(O)R^{3C}, —NR^{3A}C(O)R^{3C}, —OR^{3D}, —NR^{3A}SO₂R^{3D}, unsubstituted C₂-C₆ saturated alkyl-NR^{3E}— unsubstituted C₂-C₆ saturated alkyl, N(R^{3H})SO₂(unsubstituted C₁-C₆ saturated alkyl)-N(R^{3H}(R^{3I}), N(R^{3H})CO(unsubstituted C₁-C₆ saturated alkyl)-N(R^{3H}(R^{3I}), unsubstituted C₁-C₆ saturated alkyl, unsubstituted C₂-C₆ alkenyl, unsubstituted C₂-C₆ alkynyl, unsubstituted C₁-C₆ saturated alkylthio, or unsubstituted C₁-C₆ cycloalkyl.

In embodiments, R^{3A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy.

In embodiments, R^{3B}, R^{3C}, and R^{3D} are independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R³¹-substituted or unsubstituted phenyl, R³¹-substituted or unsubstituted heteroaryl, R³¹-substituted or unsubstituted heterocycloalkyl, or R³¹-substituted or unsubstituted heteroaryl; wherein said R³¹-substituted or unsubstituted phenyl, R³¹-substituted or unsubstituted heteroaryl, R³¹-substituted or unsubstituted heterocycloalkyl, and R³¹-substituted or unsubstituted heteroaryl are optionally substituted with one or more R³¹ substituents.

In embodiments, R³¹ is independently halogen, CN, CF₃, unsubstituted C₁-C₄ saturated alkyl, halo-substituted (saturated C₁-C₄ alkyl), unsubstituted C₂-C₄ alkenyl, unsubstituted C₂-C₄ alkynyl, or unsubstituted C₃-C₆ cycloalkyl.

In embodiments, R^{3J} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, substituted or unsubstituted aryl, substituted or unsubstituted heteroaryl, or substituted or unsubstituted heterocycloalkyl, wherein said substituted aryl, substituted heteroaryl, and substituted heterocyclic groups are independently substituted with one or more halogen, CN, CF₃, or unsubstituted C₁-C₄ saturated alkyl.

In embodiments, R^{3E} is independently hydrogen, —C(O)R^{3F}, —C(O)OR^{3F}, —SOR^{3F}, —SO₂R^{3F}, —C(O)NR^{3F}R^{3G}, or —SO₂NR^{3F}R^{3G}.

In embodiments, R^{3G} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy.

In embodiments, R^{3F} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R³¹-substituted or unsubstituted phenyl, R³¹-substituted or unsubstituted heteroaryl, R^{3I}-substituted or unsubstituted heterocycloalkyl, or R³¹-substituted or unsubstituted heteroaryl; wherein said R³¹-substituted or unsubstituted phenyl, R³¹-substituted or unsubstituted heteroaryl, R³¹-substituted or unsubstituted heterocycloalkyl, and R³¹-substituted or unsubstituted heteroaryl are optionally substituted with one or more R³¹ substituents.

In embodiments, R^{3H} and R^{3I} are independently hydrogen, substituted or unsubstituted C₁-C₆ saturated alkyl, wherein said substituted saturated alkyl is independently substituted with one or more halogen, OH, amine, CN, CF₃ or unsubstituted C₁-C₄ saturated alkyl; or R^{3H} and R^{3I}, taken together with the atom or atoms through which they are connected to form a substituted or unsubstituted 5-6 membered heterocycloalkyl or substituted or unsubstituted 5-6 membered heteroaryl, wherein the substituted 5-6 membered heterocycloalkyl or substituted 5-6 membered heteroaryl is independently substituted with one or more halogen, OH, amine, CN, CF₃, or unsubstituted C₁-C₄ saturated alkyl, wherein said amine substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy;

In embodiments, R⁴ is independently halogen, —OH, —CN, —CF₃, —C(O)NH₂, —C(O)OR^{4J}, —O—P(O)(OH)₂, —NR^{4A}R^{4B}, —C(O)R^{4C}, —NR^{4A}C(O)R^{4C}, —OR, —NR^{4A}SO₂R^{4D}, unsubstituted C₂-C₆ saturated alkyl-NR^{4E}— unsubstituted C₂-C₆ saturated alkyl, N(R^{4H})SO₂(unsubstituted C₁-C₆ saturated alkyl)-N(R^{4H}(R^{4I}), N(R^{4H})CO(unsubstituted C₁-C₆ saturated alkyl)-N(R^{4H}) R^{4I}), unsubstituted C₁-C₆ saturated alkyl, unsubstituted C₂-C₆ alkenyl, unsubstituted C₂-C₆ alkynyl, unsubstituted C₁-C₆ saturated alkylthio, or unsubstituted C₁-C₆ cycloalkyl.

In embodiments, R^{4A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy.

In embodiments, R^{4B}, R^{4C}, and R^{4D} are independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R⁴¹-substituted or unsubstituted phenyl, R⁴¹-substituted or unsubstituted heteroaryl, R⁴¹-substituted or unsubstituted heterocycloalkyl, or R⁴¹-substituted or unsubstituted heteroaryl; wherein said R⁴¹-substituted or unsubstituted phenyl, R⁴¹-substituted or unsubstituted heteroaryl, R⁴¹-substituted or unsubstituted heterocycloalkyl, and R⁴¹-substituted or unsubstituted heteroaryl are optionally substituted with one or more R⁴¹ substituents.

In embodiments, R⁴¹ is independently halogen, CN, CF₃, unsubstituted C₁-C₄ saturated alkyl, halo-substituted (saturated C₁-C₄ alkyl), unsubstituted C₂-C₄ alkenyl, unsubstituted C₂-C₄ alkynyl, or unsubstituted C₃-C₆ cycloalkyl.

In embodiments, R^{4J} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, substituted or unsubstituted aryl, substituted or unsubstituted heteroaryl, or substituted or unsubstituted heterocycloalkyl, wherein said substituted aryl, substituted heteroaryl, and substituted heterocyclic groups are independently substituted with one or more halogen, CN, CF₃, or unsubstituted C₁-C₄ saturated alkyl.

In embodiments, R^{4E} is independently hydrogen, —C(O)R^{4F}, —C(O)OR^{4F}, —SOR^{4F}, —SO₂R^{4F}, —C(O)NR^{4F}R^{4G}, or —SO₂NR^{4F}R^{4G}.

In embodiments, R^{4G} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy.

In embodiments, R^{4F} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R⁴¹-substituted or unsubstituted phenyl, R⁴¹-substituted or unsubstituted heteroaryl, R⁴¹-substituted or unsubstituted heterocycloalkyl, or R⁴¹-substituted or unsubstituted heteroaryl; wherein said R⁴¹-substituted or unsubstituted phenyl, R⁴¹-substituted or unsubstituted heteroaryl, R⁴¹-substituted or unsubstituted heterocycloalkyl, and R⁴¹-substituted or unsubstituted heteroaryl are optionally substituted with one or more R⁴¹ substituents.

In embodiments, R^{4H} and R^{4I} are independently hydrogen, substituted or unsubstituted C₁-C₆ saturated alkyl, wherein said substituted saturated alkyl is independently substituted with one or more halogen, OH, amine, CN, CF₃, or unsubstituted C₁-C₄ saturated alkyl; or R^{4H} and R^{4I}, taken together with the atom or atoms through which they are connected to form a substituted or unsubstituted 5-6 membered heterocycloalkyl or substituted or unsubstituted 5-6 membered heteroaryl, wherein the substituted 5-6 membered heterocycloalkyl or substituted 5-6 membered heteroaryl is independently substituted with one or more halogen, OH, amine, CN, CF₃, or unsubstituted C₁-C₄ saturated alkyl, wherein said amine substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy.

In embodiments, L is halo-substituted (C₁-C₁₀ saturated alkylene), R^{L}-substituted or unsubstituted C₁-C₁₀ alkylene, R^{L}-substituted or unsubstituted C₂-C₁₀ alkenylene, R^{L}-substituted or unsubstituted C₂-C₁₀ alkynylene, R^{L}-substituted or unsubstituted C₂-C₆ saturated alkylene-O—C₂-C₆ saturated alkylene, R^{L}-substituted or unsubstituted C₂ saturated alkylene-O—C₂ saturated alkylene, R^{L}-substituted or unsubstituted C₂ saturated alkylene-O—C₂ saturated alkylene-O—C₂ saturated alkylene, R^{L}-substituted or unsubstituted C₂-C₆ saturated alkylene-NR^{L4}—C₂-C₆ saturated alkylene, R^{L}-substituted or unsubstituted C₃-C₆ cycloalkylene, R^{L}-substituted or unsubstituted phenylene, R^{L}-substituted or unsubstituted 4-6 membered heterocycloalkylene, R^{L}-substituted or unsubstituted 5-6 membered heteroarylene, R^{L}-substituted or unsubstituted C₁-C₄ saturated alkylene-(C₃-C₆ cycloalkylene)-C₁-C₄ saturated alkylene, R^{L}-substituted or unsubstituted C₁-C₄ saturated alkylene-phenylene-C₁-C₄ saturated alkylene, R^{L}-substituted or unsubstituted C₁-C₄ saturated alkylene-(4-6 membered heterocycloalkylene)-C₁-C₄ saturated alkylene, or R^{L}-substituted or unsubstituted C₁-C₄ saturated alkylene-(5-6 membered heteroarylene)-C₁-C₄ saturated alkylene;

In embodiments, said R^{L}-substituted C₁-C₁₀alkyl, R^{L}-substituted C₂-C₁₀ alkenyl, R^{L}-substituted C₂-C₁₀ alkynyl, R^{L}-substituted C₂-C₆ saturated alkyl-O—C₂-C₆ saturated alkyl, R^{L}-substituted C₂ saturated alkyl-O—C₂ saturated alkyl, R^{L}-substituted C₂ saturated alkyl-O—C₂ saturated alkyl-O—C₂ saturated alkyl, R^{L}-substituted C₂-C₆ saturated alkyl-NR⁴—C₂-C₆ saturated alkyl, R^{L}-substituted C₃-C₆ cycloalkyl, R^{L}-substituted phenyl, R^{L}-substituted 4-6 membered heterocycloalkyl, R^{L}-substituted 5-6 membered heteroaryl, R^{L}-substituted C₁-C₄ saturated alkyl-(C₃-C₆ cycloalkyl)-C₁-C₄ saturated alkyl, R^{L}-substituted C₁-C₄ saturated alkyl-phenyl-C₁-C₄ saturated alkyl, R^{L}-substituted C₁-C₄ saturated alkyl-(4-6 membered heterocycloalkyl)-C₁-C₄ saturated alkyl, and R^{L}-substituted C₁-C₄ saturated alkyl-(5-6 membered heteroaryl)-C₁-C₄ saturated alkyl are independently substituted with 1 or 2 R^{L}.

In embodiments, R^{L} is independently halogen, —OR^{LB}, —O—P(O)(OR^{LA})₂, —NR^{LA}R^{LB}, —OC(O)R^{LB}, —C(O)—OR^{LB}, —SOR^{LB}, —SO₂R^{LB}, —C(O)NR^{LA}R^{LB}, —SO₂NR^{L A}R^{LB}, —OC(O)NR^{LA}R^{LB}, —NR^{LA}C(O)R^{LB}, —NR^{LA}SOR^{LB}, —NR^{LA}C(O)OR^{LB}, —NR^{LA}SO₂R^{LB}, —R^{LA}S O₂R^{LB}, halo-substituted (saturated C₁-C₄alkyl), (unsubstituted C₁-C₄ saturated alkyl)(unsubstituted C₁-C₄ saturated alkyl)amino, unsubstituted C₁-C₄ saturated alkyl, hydroxy-substituted (C₁-C₄ saturated alkyl), (unsubstituted C₁-C₄ saturated alkyl)-O—P(O)(OH)₂, (unsubstituted C₁-C₄ saturated alkyl)-O—P(O)OR^{LA})₂, halo-substituted (C₁-C₄ alkoxy), unsubstituted C₁-C₄ alkoxy, unsubstituted C₁-C₆ saturated alkylthio, hydroxy-substituted (C₂-C₄ alkoxy), (unsubstituted C₂-C₄ alkoxy)-O—P(O)(OH)₂, (unsubstituted C₂-C₄alkoxy)-O—P(O)(OR^{LA})₂, unsubstituted C₁-C₄ alkoxy(unsubstituted C₁-C₄ alkoxy), COR^{LA}, or CON(R^{LA})(R^{LC}).

In embodiments, R^{LA} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy.

In embodiments, R^{LB} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R^{L6}-substituted or unsubstituted phenyl, R^{L6}-substituted or unsubstituted heteroaryl, R^{L6}-substituted or unsubstituted heterocycloalkyl, or R^{L}-substituted or unsubstituted heteroaryl; wherein said R^{L6}-substituted or unsubstituted phenyl, R^{L6}-substituted or unsubstituted heteroaryl, R^{L6}-substituted or unsubstituted heterocycloalkyl, and R^{L6}-substituted or unsubstituted heteroaryl are optionally substituted with one or more R^{L6} substituents.

In embodiments, R^{L6} is independently halogen, CN, CF₃, unsubstituted C₁-C₄ saturated alkyl, halo-substituted (saturated C₁-C₄ alkyl), unsubstituted C₂-C₄ alkenyl, unsubstituted C₂-C₄ alkynyl, or unsubstituted C₃-C₆ cycloalkyl.

In embodiments, R^{LC}—OR^{4D}, is independently hydrogen, substituted or unsubstituted C₁-C₆ saturated alkyl, wherein said substituted saturated alkyl is independently substituted with one or more halogen, OH, amine, CN, CF₃, or unsubstituted C₁-C₄ saturated alkyl.

In embodiments, R^{L4} is independently hydrogen, COR^{L4A}, CO₂H, CO₂R^{L4A}, SOR^{L4A}, SO₂R^{L4A}, CONH₂, CONR^{L4A}R^{L4B}, SO₂NH₂, or SO₂NR^{L4A}R^{L4B}.

In embodiments, R^{L4B} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy.

In embodiments, R^{L4A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R^{L6}-substituted or unsubstituted phenyl, R^{L6}-substituted or unsubstituted heterocycloalkyl, or R^{L6}-substituted or unsubstituted heteroaryl; wherein said R^{L6}-substituted or unsubstituted phenyl, R^{L6}-substituted or unsubstituted heterocycloalkyl, and R^{L6}-substituted or unsubstituted heteroaryl are optionally substituted with one or more R^{L6} substituents.

A⁹ is independently N or CR⁹.

In embodiments, R⁹ is independently hydrogen, halogen, —CN, —CF₃, O—P(O)(OR^{9A})₂, —OR^{9D}, —NR^{9B}R^{9B}, —NR^{9A}R^{9B}, —OC(O)NR^{9A}R^{9B}, —OC(O)R^{9C}, —C(O)R^{9C}, —C(O)—OR^{9J}, —C(O)NR^{9A}R^{9B}, —SOR^{9D}, —SO₂NR^{9A}R^{9B}, —SO₂R^{9J}, —NR^{9A}SO₂R^{9D}, —NR^{9A}C(O)R^{9C}, N(R^{9H})SO₂(unsubstituted C₁-C₄ saturated alkyl)-N(R^{9I})(R^{9H}), N(R^{9H})CO(unsubstituted C₁-C₄ saturated alkyl)-N(R^{9I})R^{9H}), O(unsubstituted C₁-C₄ saturated alkyl)-N(R^{9I}(R^{9H}), O(unsubstituted C₁-C₄ saturated alkyl)-OR^{9B}, NR^{9H}(unsubstituted C₁-C₄ saturated alkyl)-N(R^{9I})(R^{9H}), halo-substituted (C₁-C₁₀ saturated alkyl), halo-substituted (C₁-C₁₀ alkoxy), R⁹⁰-substituted or unsubstituted (C₁-C₁₀ saturated alkyl)oxy, R⁹⁰-substituted or unsubstituted (C₁-C₁₀ saturated alkyl)amino, R⁹⁰-substituted or unsubstituted (C₁-C₆ saturated alkyl)(C₁-C₄ saturated alkyl)amino, R⁹⁰-substituted or unsubstituted phenyl, R⁹⁰-substituted or unsubstituted 5-6 membered heterocycloalkyl, or R⁹⁰-substituted or unsubstituted 5-6 membered heteroaryl.

In embodiments, R^{9A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy.

In embodiments, R^{9B}, R^{9C}, and R^{9D} are independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R⁹¹-substituted or unsubstituted phenyl, R⁹¹-substituted or unsubstituted heteroaryl, R⁹¹-substituted or unsubstituted heterocycloalkyl, or R⁹¹-substituted or unsubstituted heteroaryl; wherein said R⁹¹-substituted or unsubstituted phenyl, R⁹¹-substituted or unsubstituted heteroaryl, R⁹¹-substituted or unsubstituted heterocycloalkyl, and R⁹¹-substituted or unsubstituted heteroaryl are optionally substituted with one or more R⁹¹ substituents.

In embodiments, R⁹¹ is independently halogen, CN, CF₃, unsubstituted C₁-C₄ saturated alkyl, halo-substituted (saturated C₁-C₄ alkyl), unsubstituted C₂-C₄ alkenyl, unsubstituted C₂-C₄ alkynyl, or unsubstituted C₃-C₆ cycloalkyl.

In embodiments, R^{9J} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, substituted or unsubstituted aryl, substituted or unsubstituted heteroaryl, or substituted or unsubstituted heterocycloalkyl, wherein said substituted aryl, substituted heteroaryl, and substituted heterocyclic groups are independently substituted with one or more halogen, CN, CF₃, or unsubstituted C₁-C₄ saturated alkyl.

In embodiments, R^{9H} and R^{9I} are independently hydrogen, substituted or unsubstituted C₁-C₆ saturated alkyl, wherein said substituted saturated alkyl is independently substituted with one or more halogen, OH, amine, CN, CF₃, or unsubstituted C₁-C₄ saturated alkyl; or R^{9H} and R^{9I}, taken together with the atom or atoms through which they are connected to form a substituted or unsubstituted 5-6 membered heterocycloalkyl or substituted or unsubstituted 5-6 membered heteroaryl, wherein the substituted 5-6 membered heterocycloalkyl or substituted 5-6 membered heteroaryl is independently substituted with one or more halogen, OH, amine, CN, CF₃, or unsubstituted C₁-C₄ saturated alkyl, wherein said amine substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy.

In embodiments, said R⁹⁰-substituted (C₁-C₁₀ saturated alkyl)oxy, R⁹⁰-substituted (C₁-C₁₀ saturated alkyl)amino, R⁹⁰-substituted (C₁-C₆ saturated alkyl)(C₁-C₄ saturated alkyl)amino, R⁹⁰-substituted phenyl, R⁹⁰-substituted 5-6 membered heterocycloalkyl, or R⁹⁰-substituted 5-6 membered heteroaryl is substituted with 1-4 independent R⁹⁰ and

In embodiments, R⁹⁰ is halogen, —OR^{90B}, —O—P(O)(OR^{90A})₂, —NR^{90A}R^{9B}, —NR^{90B}R^{90B}, —C(O)—OR^{90B}, —C(O)NR^{90A}R^{90B}, —SOR^{90B}, —SO₂R^{90B}, —SO₂NR^{90A}R^{90B}, —OC(O)R^{90B}, —OC(O)NR^{90A}R^{90B}, —NR^{90A}C(O)R^{90B}, —NR^{90A}SOR^{90B}, —NR^{90A}C(O)OR^{90B}, —C(O)R^{90B}, —SO₂R^{90J}, —C(O)—OR^{90J}, —C(O)NR^{90A}R^{90H}, —C(O)R^{90A}, (unsubstituted C₁-C₄ saturated alkyl)(unsubstituted C₁-C₄ saturated alkyl)amino, unsubstituted C₁-C₄ saturated alkyl, halo-substituted (C₁-C₄ saturated alkyl), hydroxy-substituted (C₁-C₄ saturated alkyl), (unsubstituted C₁-C₄ saturated alkyl)-O—P(O)(OH)₂, (unsubstituted C₁-C₄ saturated alkyl)-O—P(O)(OR^{90A})₂, halo-substituted (C₁-C₄ alkoxy), unsubstituted C₁-C₄ alkoxy, hydroxy-substituted (C₂-C₄ alkoxy), (unsubstituted C₂-C₄ alkoxy)-O—P(O)(OH)₂, (unsubstituted C₂-C₄alkoxy)-O—P(O)(OR^{90A})₂, or unsubstituted C₁-C₄ alkoxy-(unsubstituted C₁-C₄ alkoxy).

In embodiments, R^{90A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy.

In embodiments, R^{90B} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R⁹¹-substituted or unsubstituted phenyl, R⁹¹-substituted or unsubstituted heteroaryl, R⁹¹-substituted or unsubstituted heterocycloalkyl, or R⁹¹-substituted or unsubstituted heteroaryl; wherein said R⁹¹-substituted or unsubstituted phenyl, R⁹¹-substituted or unsubstituted heteroaryl, R⁹¹-substituted or unsubstituted heterocycloalkyl, and R⁹¹-substituted or unsubstituted heteroaryl are optionally substituted with one or more R⁹¹ substituents.

In embodiments, R⁹¹ is independently halogen, CN, CF₃, unsubstituted C₁-C₄ saturated alkyl, halo-substituted (saturated C₁-C₄ alkyl), unsubstituted C₂-C₄ alkenyl, unsubstituted C₂-C₄ alkynyl, or unsubstituted C₃-C₆ cycloalkyl.

In embodiments, R^{90J} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, substituted or unsubstituted aryl, substituted or unsubstituted heteroaryl, or substituted or unsubstituted heterocycloalkyl, wherein said substituted aryl, substituted heteroaryl, and substituted heterocyclic groups are independently substituted with one or more halogen, CN, CF₃, or unsubstituted C₁-C₄ saturated alkyl.

In embodiments, R^{90H} is independently hydrogen, substituted or unsubstituted C₁-C₆ saturated alkyl, wherein said substituted saturated alkyl is independently substituted with one or more halogen, OH, amine, CN, CF₃, or unsubstituted C₁-C₄ saturated alkyl, wherein said amine substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy; or a tautomer thereof, or a salt thereof (e.g., pharmaceutically acceptable salt).

In embodiments (e.g., of Formula I), A⁹ is independently CR⁹ and R⁹ is as described herein, including in embodiments.

In embodiments (e.g., of Formula I), A⁹ is independently CR⁹ and R⁹ is methoxy.

In embodiments (e.g., of Formula I), A⁹ is independently CR⁹ and R⁹ is:

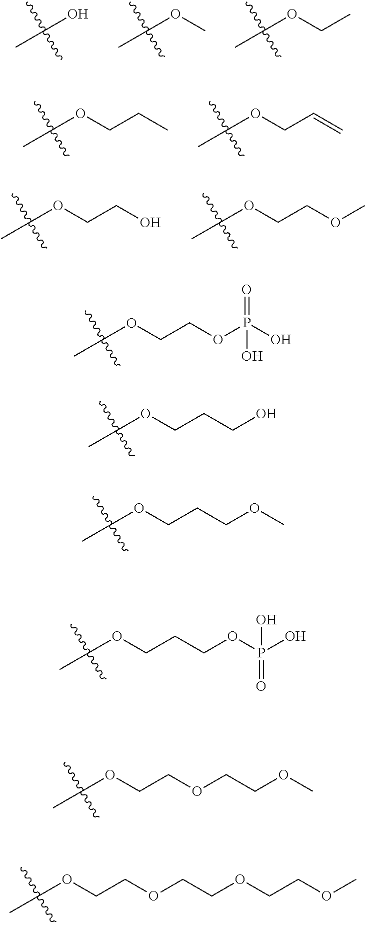

In embodiments (e.g., of Formula I), A⁹ is independently CR⁹ and R⁹ is:

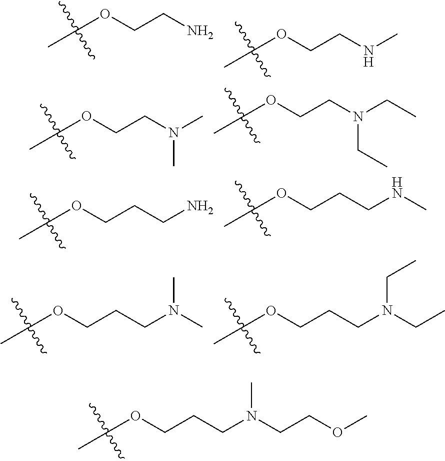

In embodiments (e.g., of Formula I), A⁹ is independently CR⁹ and R⁹ is:

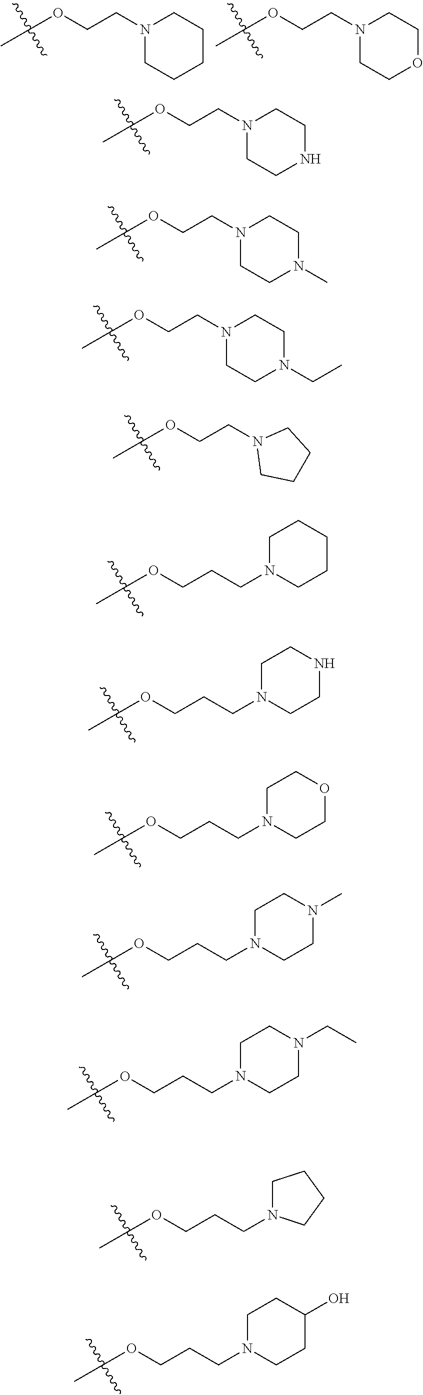

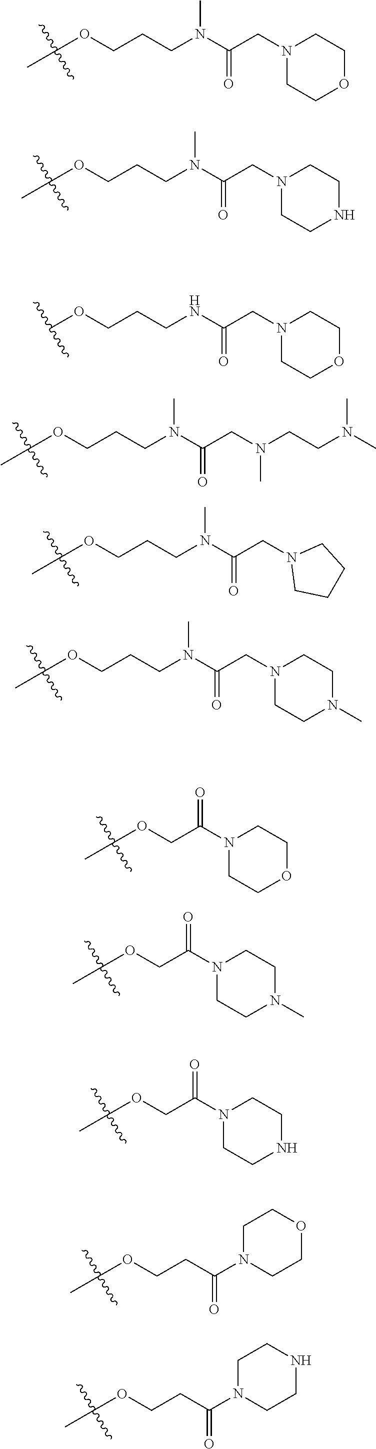

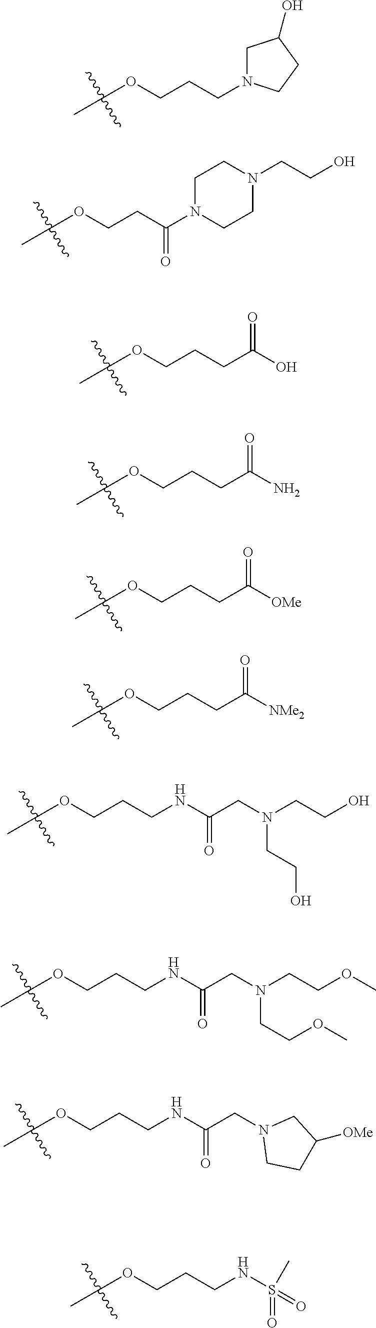

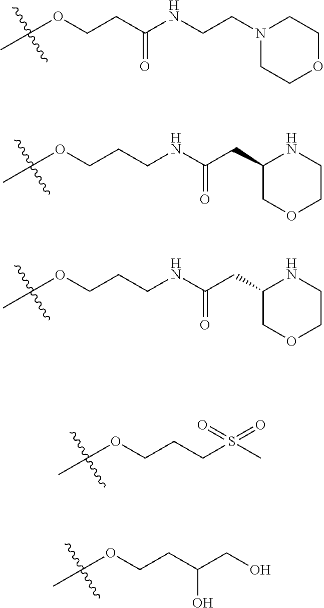

In embodiments (e.g., of Formula I), Lis:

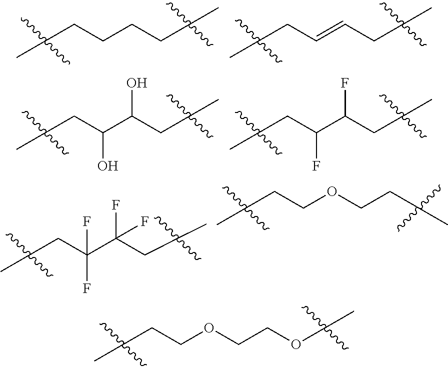

In embodiments, R³ and R⁴ are hydrogen. In embodiments, R³ is —C(O)NR^{3A}R^{3B}. In embodiments, R³ is —C(O)NH₂. In embodiments, R⁴ is —C(O)NR^{4A}R^{4B}. In embodiments, R⁴ is —C(O)NH₂. In embodiments, R³ and R⁴ are —C(O)NH₂.

It will also be appreciated by those skilled in the art that in embodiments, the compounds of this invention of Formula I may exist in tautomeric forms including, but not limited to, Formula (A), Formula (B) and/or Formula (C) or zwitterionic forms including, but not limited to, Formula (D) or Formula (E)

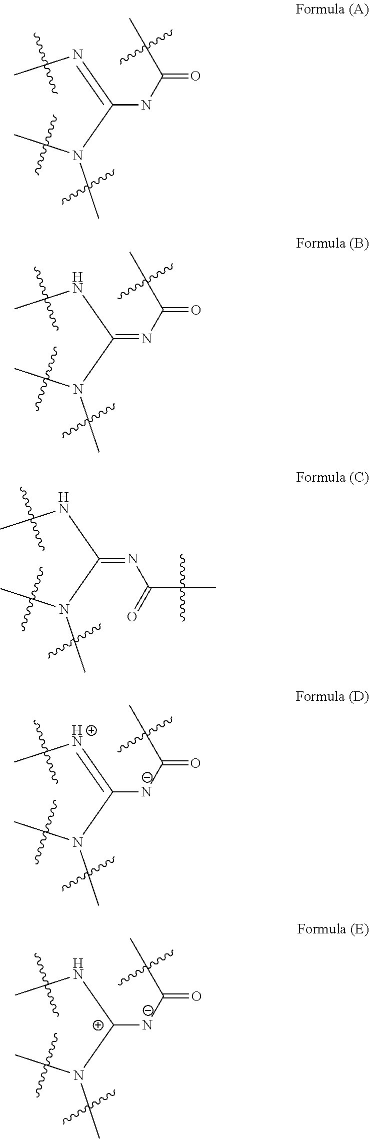

In certain embodiments, the compounds of this invention of Formula Ie may exist in tautomeric forms including, but not limited to, Formula (Ie-A), Formula (Ie-B), Formula (Ie-C), Formula (Ie-D) and/or Formula (Ie-F) or zwitterionic forms:

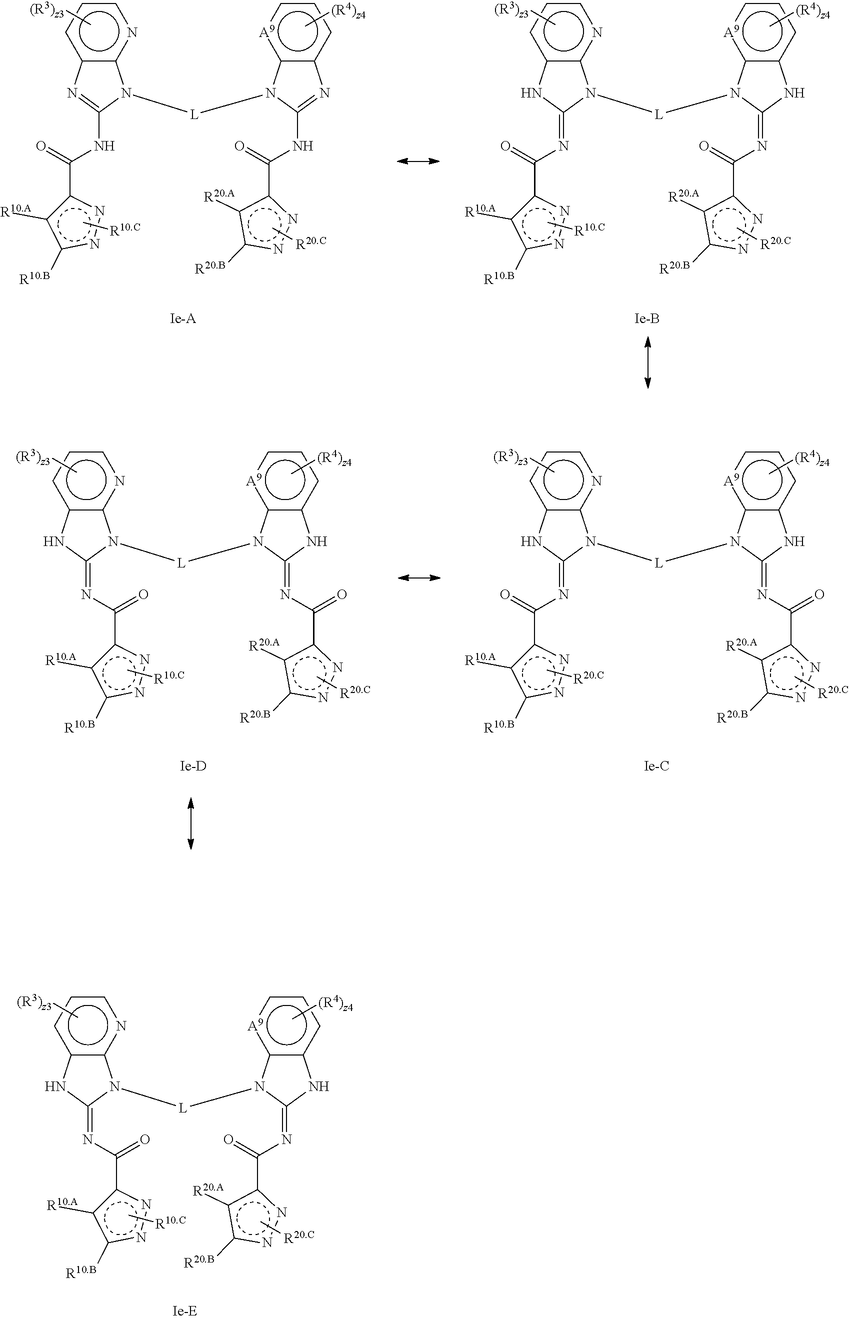

Wherein A⁹ is as described herein.

R³, z3, R⁴, z4, R^{10.A}, R^{10.B}, R^{10.C}, R^{20.A}, R^{20.B}, R^{20.C}, A⁹, and L are as described herein.

In embodiments, (e.g., the compound of Formula (Ia), (Ib), (Ic) or (Id)), R¹ and R² are independently azoyl.

In embodiments, (e.g., the compound of Formula (Ia), (Ib), (Ic) or (Id)), R¹ and R² are independently:

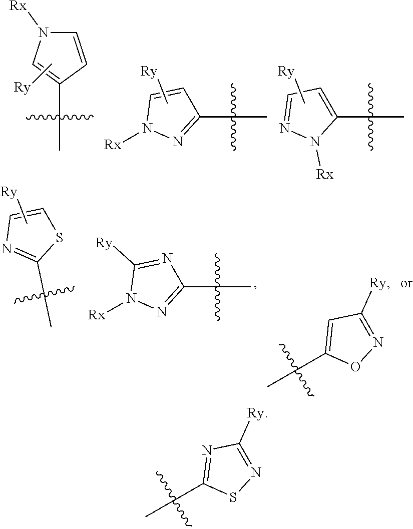

wherein R_{y} is independently hydrogen, deutero, halo, unsubstituted saturated alkyl, cyano, haloalkyl, unsubstituted cycloalkyl, hydroxy, or unsubstituted alkoxy; and each Rₓ is independently hydrogen, or unsubstituted saturated alkyl.

In embodiment, (e.g., the compound of Formula (Ia), (Ib), (Ic) or (Id)), R¹ and R² are independently:

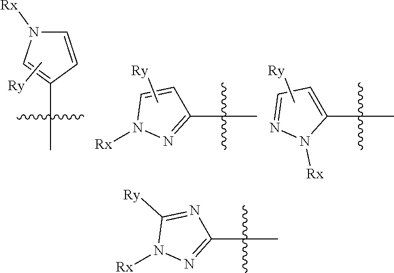

wherein R_{y} is independently cyano, unsubstituted saturated alkyl, unsubstituted alkenyl, unsubstituted alkynyl, unsubstituted cycloalkyl, unsubstituted aryl, unsubstituted heteroaryl, unsubstituted heterocycloalkyl, or unsubstituted heterocyclyl, —OR^{10B}, —NR^{10A}R^{10B}; and each Rₓ is independently hydrogen or unsubstituted saturated alkyl.

In one embodiment, the compound of Formula (Ia), (Ib), (Ic) or (Id), wherein R′ and R² represent each independently

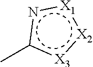

wherein X₁, X₂, X₃ are independently selected from N, S, O or CR_{y}.

In one embodiment, the compound of Formula (Ia), (Ib), (Ic) or (Id), wherein R¹ and R² are independently

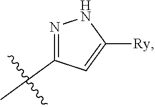

herein R_{y} is independently cyano, unsubstituted saturated alkyl, unsubstituted alkenyl, unsubstituted alkynyl, unsubstituted cycloalkyl, unsubstituted aryl, unsubstituted heteroaryl, unsubstituted heterocycloalkyl, or unsubstituted heterocyclyl, —OR^{10B}, or —NR^{10A}R^{10B}.

In embodiment, (e.g., the compound of Formula (Ia), (Ib), (Ic), (Id) or (Ie)), R³ and R⁴ are independent hydrogen, halogen, COOH, or CONH₂.

In embodiment, (e.g., the compound of Formula (Ia), (Ib), (Ic), (Id) or (Ie), A⁹ is N.

In embodiment, (e.g., the compound of Formula (Ta), (Ib), (Ic), (Id) or (Ie), A⁹ is CR⁹, and R⁹ is OMe.

In embodiment, (e.g., the compound of Formula (Ia), (Ib), (Ic), (Id) or (Ie), A⁹ is CR⁹, and R⁹ is OR^{9D}. In embodiment, (e.g., the compound of Formula (Ia), (Ib), (Ic), (Id) or (Ie), A⁹ is CR⁹, and R⁹ is OMe.

In embodiment, (e.g., the compound of Formula (Ia), (Ib), (Ic), (Id) or (Ie), L is halo(C₁-C₁₀ saturated alkyl), unsubstituted C₁-C₁₀ saturated alkyl, unsubstituted C₂-C₁₀ alkenyl, unsubstituted C₂ saturated alkyl-O-unsubstituted C₂ saturated alkyl, unsubstituted C₂ saturated alkyl-O— unsubstituted C₂ saturated alkyl-O— unsubstituted C₂ saturated alkyl, unsubstituted C₂-C₆ saturated alkyl-R^{L4}-unsubstituted C₂-C₆ saturated alkyl, and unsubstituted C₃-C₆cycloalkyl.

In embodiment, (e.g., the compound of Formula (Ia), (Ib), (Ic), (Id) or (Ie), L is halo(C₁-C₁₀ saturated alkyl), unsubstituted C₁-C₁₀ saturated alkyl, unsubstituted C₂-C₁₀ alkenyl and unsubstituted C₂ saturated alkyl-O— unsubstituted C₂ saturated alkyl.

In embodiment, (e.g., the compound of Formula (Ia), (Ib), (Ic), (Id) or (Ie), L is C₄ alkenyl.

In embodiments, A⁹ is independently N or CR⁹; R⁹ is independently R⁹⁰-substituted or unsubstituted heteroalkyl; R⁹⁰ is —NR^{90A}R^{90B}; R^{90A} and R^{90B} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —COH, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R⁹¹-substituted or unsubstituted saturated alkyl, R⁹¹-substituted or unsubstituted alkenyl, R⁹¹-substituted or unsubstituted alkynyl, R⁹¹-substituted or unsubstituted heteroalkyl, R⁹¹-substituted or unsubstituted cycloalkyl, R⁹¹-substituted or unsubstituted heterocycloalkyl, R⁹¹-substituted or unsubstituted aryl, or R⁹¹-substituted or unsubstituted heteroaryl; R^{90A} and R^{90B} substituents bonded to the same nitrogen atom may optionally be joined to form a R⁹¹-substituted or unsubstituted heterocycloalkyl or R⁹¹-substituted or unsubstituted heteroaryl; R⁹¹-substituted or unsubstituted saturated alkyl, R⁹¹-substituted or unsubstituted alkenyl, R⁹¹-substituted or unsubstituted alkynyl, R⁹¹-substituted or unsubstituted heteroalkyl, R⁹¹-substituted or unsubstituted cycloalkyl, R⁹¹-substituted or unsubstituted heterocycloalkyl, R⁹¹-substituted or unsubstituted aryl, or R⁹¹-substituted or unsubstituted heteroaryl; R^{90A} and R^{90B} substituents bonded to the same nitrogen atom may optionally be joined to form a R⁹¹-substituted or unsubstituted heterocycloalkyl or R⁹¹-substituted or unsubstituted heteroaryl; R⁹¹ is independently oxo, halogen, —OR^{91B}, —O—P(O)(OR^{91A})₂, —NR^{91A}R^{91B}, —NR^{91A}R^{91A}, —C(O)—OR^{91B}, —C(O)NR^{91A}R^{91B}, —SOR^{91B}, —SO₂R^{91B}, —SO₂NR^{91A}R^{9B}, —OC(O)R^{91B}, —OC(O)NR^{91A}R^{91B}, —NR^{91A}C(O)R^{91B}, NR^{91A}SOR^{9B}, —NR^{91A}C(O)OR^{91B}, —C(O)R^{91B}, —CN, —CX⁹¹₃, —CHX⁹¹₂, —CH₂X⁹¹, NR^{91A}SO₂R^{91B}, —OCX⁹¹₃, —OCH₂X⁹¹, —OCHX⁹¹₂, —SR^{91B}, —NR^{91A}R^{91B} ONR^{91A}R^{91B}, —NHC(O)NHNR^{9A}R^{91B}, —NHC(O)NR^{91A}R^{91B}, —N(O)₂, —NR^{91A}OR^{91B}, —SF₅, —N₃, -(unsubstituted C₁-C₄ saturated alkyl)-O—P(O)(OH)₂, -(unsubstituted C₁-C₄ saturated alkyl)-O—P(O)(OR^{91A})₂, -(unsubstituted C₂-C₄ alkoxy)-O—P(O)(OH)₂, -(unsubstituted C₂-C₄alkoxy)-O—P(O)(OR^{91A})₂, R⁹²-substituted or unsubstituted saturated alkyl, R⁹²-substituted or unsubstituted alkenyl, R⁹²-substituted or unsubstituted alkynyl, R⁹²-substituted or unsubstituted heteroalkyl, R⁹²-substituted or unsubstituted cycloalkyl, R⁹²-substituted or unsubstituted heterocycloalkyl, R⁹²-substituted or unsubstituted aryl, or R⁹²-substituted or unsubstituted heteroaryl; R⁹² is independently oxo, halogen, —CCl₃, —CBr₃, —CF₃, —CI₃, —CHCl₂, —CHBr₂, —CHF₂, —CHI₂, —CH₂C₁, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, unsubstituted alkyl, unsubstituted heteroalkyl, unsubstituted cycloalkyl, unsubstituted heterocycloalkyl, unsubstituted aryl, or unsubstituted heteroaryl.

R^{91A}, R^{91B}, and R^{91C} are independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, R⁹²-substituted or unsubstituted saturated alkyl, Rn-substituted or unsubstituted alkenyl, R²-substituted or unsubstituted alkynyl, R⁹²-substituted or unsubstituted heteroalkyl, R⁹²-substituted or unsubstituted cycloalkyl, Rn-substituted or unsubstituted heterocycloalkyl, R⁹²-substituted or unsubstituted aryl, or R⁹²-substituted or unsubstituted heteroaryl.

In embodiments, R⁹ is independently R⁹⁰-substituted or unsubstituted alkoxy.

In embodiments, R⁹ is independently R⁹⁰-substituted or unsubstituted C₁-C₈ alkoxy.

In embodiments, R⁹ is independently R⁹⁰-substituted or unsubstituted C₁-C₆ alkoxy.

In embodiments, R⁹ is independently R⁹⁰-substituted or unsubstituted C₂-C₆ alkoxy. In embodiments, R⁹ is independently OH-substituted or unsubstituted C₂-C₃ alkoxy. In embodiments, R⁹ is independently —OH. In embodiments, R⁹ is independently unsubstituted C₂-C₃ alkoxy. In embodiments, R⁹ is independently OP(O)(OH)₂-substituted or unsubstituted C₂-C₃ alkoxy. In embodiments, R⁹ is independently -(unsubstituted alkoxy)-O— P(O)(OR^{9A})₂. I embodiments, R⁹ is independently —OR^{9D}. In embodiments, R⁹ is independently —O(unsubstituted C₁-C₄ alkyl)-NR^{9A}R^{9B}. In embodiments, R⁹ is independently —O(unsubstituted C₁-C₄ alkyl)-OR^{9D}. In embodiments, R⁹ is independently —O(unsubstituted C₁-C₄ alkyl)-OR^{9B}. In embodiments, R⁹ is independently R⁹⁰-substituted or unsubstituted heteroalkyl. In embodiments, R^{9A} is independently hydrogen. In embodiments, R^{9A} is independently R⁹⁰-substituted or unsubstituted saturated alkyl (e.g., C₁-C₈, C₁-C₆, C₁-C₄, or C₁-C₂). In embodiments, R^{B} is independently hydrogen. In embodiments, R^{B} is independently R⁹⁰-substituted or unsubstituted saturated alkyl (e.g., C₁-C₈, C₁-C₆, C₁-C₄, or C₁-C₂). In embodiments, R^{9C} is independently hydrogen. In embodiments, R^{9C} is independently R⁹⁰-substituted or unsubstituted saturated alkyl (e.g., C₁-C₈, C₁-C₆, C₁-C₄, or C₁-C₂). In embodiments, R^{9D} is independently hydrogen. In embodiments, R^{9D} is independently R⁹⁰-substituted or unsubstituted saturated alkyl (e.g., C₁-C₈, C₁-C₆, C₁-C₄, or C₁-C₂). In embodiments, R^{9A} and R^{9B} substituents bonded to the same nitrogen atom may optionally be joined to form a R⁹⁰-substituted or unsubstituted heterocycloalkyl (e.g., 3 to 8 membered, 3 to 6 membered, 4 to 6 membered, 4 to 5 membered, or 5 to 6 membered). In embodiments, R⁹⁰ is independently —NR^{90B}R^{90B}. In embodiments, R⁹⁰ is independently —C(O)—OR^{90B}. In embodiments, R⁹⁰ is independently —C(O)NR^{90A}R^{90B}. In embodiments, R⁹⁰ is independently —C(O)R^{90B}. In embodiments, R⁹⁰ is independently R⁹¹-substituted or unsubstituted C₁-C₆ saturated alkyl. In embodiments, R⁹⁰ is independently R⁹¹-substituted or unsubstituted heterocycloalkyl. In embodiments, R⁹⁰ is independently R⁹¹-substituted or unsubstituted heteroaryl. In embodiments, R^{90A} is independently hydrogen. In embodiments, R^{90A} is independently R⁹¹-substituted or unsubstituted saturated alkyl. In embodiments, R^{90B} is independently hydrogen. In embodiments, R^{90B} is independently R⁹¹-substituted or unsubstituted saturated alkyl. In embodiments, R⁹¹ is independently oxo. In embodiments, R⁹¹ is independently —OH. In embodiments, R⁹¹ is independently —NH₂. In embodiments, R⁹⁰ is independently R⁹²-substituted or unsubstituted C₁-C₄ saturated alkyl. In embodiments, R⁹¹ is independently unsubstituted C₃-C₆ cycloalkyl. In embodiments, R⁹¹ is independently R²-substituted or unsubstituted 3 to 6 membered heterocycloalkyl. In embodiments, R⁹¹ is independently R⁹²-substituted or unsubstituted 3 to 12 membered heterocycloalkyl. In embodiments, R⁹¹ is independently R⁹²-substituted or unsubstituted 3 to 15 membered heterocycloalkyl. In embodiments, R⁹² is independently oxo. In embodiments, R⁹² is independently —OH. In embodiments, R⁹² is independently unsubstituted alkyl. In embodiments, R^{9A} and R^{9B} substituents bonded to the same nitrogen atom are joined to form a R⁹⁰-substituted or unsubstituted morpholinyl. In embodiments, R^{9A} and R^{9B} substituents bonded to the same nitrogen atom are joined to form a R⁹⁰-substituted or unsubstituted piperidinyl. In embodiments, R^{9A} and R^{9B} substituents bonded to the same nitrogen atom are joined to form a R⁹⁰-substituted or unsubstituted piperazinyl. In embodiments, R^{9A} and R^{9B} substituents bonded to the same nitrogen atom are joined to form a R⁹⁰-substituted or unsubstituted thiomorpholinyl. In embodiments, R^{9A} and R^{9B} substituents bonded to the same nitrogen atom are joined to form a R⁹⁰-substituted or unsubstituted thiomorpholinyl dioxide. In embodiments, R⁹⁰ is independently R⁹¹-substituted or unsubstituted morpholinyl. In embodiments, R⁹⁰ is independently R⁹¹-substituted or unsubstituted piperidinyl. In embodiments, R⁹⁰ is independently R⁹¹-substituted or unsubstituted piperazinyl. In embodiments, R⁹⁰ is independently R⁹¹-substituted or unsubstituted thiomorpholinyl. In embodiments, R⁹⁰ is independently R⁹¹-substituted or unsubstituted thiomorpholinyl dioxide. In embodiments, R⁹¹ is independently R⁹²-substituted or unsubstituted morpholinyl. In embodiments, R⁹¹ is independently R⁹²-substituted or unsubstituted piperidinyl. In embodiments, R⁹¹ is independently R⁹²-substituted or unsubstituted piperazinyl. In embodiments, R⁹¹ is independently R⁹²-substituted or unsubstituted thiomorpholinyl. In embodiments, R⁹¹ is independently R⁹²-substituted or unsubstituted thiomorpholinyl dioxide.

In embodiments, R⁹⁰ is —NR^{90A}R^{90B}.

In embodiments, R^{90A} and R^{90B} are independently hydrogen, R⁹¹-substituted or unsubstituted saturated alkyl; R^{90A} and R^{90B} substituents bonded to the same nitrogen atom may optionally be joined to form a R⁹¹-substituted or unsubstituted heterocycloalkyl.

In embodiments, R^{90A} is independently R⁹¹-substituted or unsubstituted saturated alkyl.

In embodiments, R^{90B} is independently R⁹¹-substituted or unsubstituted saturated alkyl.

In embodiments, R^{90A} and R^{90B} substituents bonded to the same nitrogen atom are joined to form a R⁹¹-substituted or unsubstituted heterocycloalkyl.

In embodiments, R^{90A} and R^{90B} substituents bonded to the same nitrogen atom are joined to form a R⁹¹-substituted or unsubstituted 5 to 6 membered heterocycloalkyl.

In embodiments, R^{90A} and R^{90B} substituents bonded to the same nitrogen atom are joined to form a R⁹¹-substituted or unsubstituted morpholinyl, R⁹¹-substituted or unsubstituted piperidinyl, R⁹¹-substituted or unsubstituted piperazinyl, or R⁹¹-substituted or unsubstituted pyrrolidinyl.

In embodiments, R⁹¹ is independently oxo, —O—P(O)(OR^{91A})₂, —OR^{91B}, —NR^{91B}R^{91B}, —NR^{91B}R^{91A}, —CO₂R^{91C}, —OCOR^{91B}, —CO₂R^{91B}, —SOR^{91B}, —SO₂R^{91C}, —CONR^{91B}R^{91A}, —SO₂NR^{91B}R^{91A}, —OCONR^{91B}R^{91A}, —NR^{91A}COR^{91B}, —NR^{91A}SOR^{91B}, —NR^{91A} CO₂R^{91B}, —COR^{91A}, -(unsubstituted C₁-C₄ saturated alkyl)-O—P(O)(OH)₂, -(unsubstituted C₁-C₄ saturated alkyl)-O—P(O)OR^{91A})₂, -(unsubstituted C₂-C₄ alkoxy)-O—P(O)(OH)₂, -(unsubstituted C₂-C₄alkoxy)-O— P(O)(OR^{91A})₂, (unsubstituted C₁-C₄ saturated alkyl)(unsubstituted C₁-C₄ saturated alkyl)amino, unsubstituted C₁-C₄ alkyl, halo-substituted (C₁-C₄ alkyl), hydroxy-substituted (C₁-C₄ saturated alkyl), halo-substituted (C₁-C₄ alkoxy), unsubstituted C₁-C₄ alkoxy, hydroxy-substituted (C₂-C₄ alkoxy), C₁-C₄ alkoxy-(C₁-C₄ alkoxy), halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, R⁹²-substituted or unsubstituted C₁-C₄ saturated alkyl, R⁹²-substituted or unsubstituted C₂-C₄ alkenyl, R⁹²-substituted or unsubstituted C₂-C₄ alkynyl, R⁹²-substituted or unsubstituted 2 to 12 membered heteroalkyl, R⁹²-substituted or unsubstituted C₃-C₆ cycloalkyl, R⁹²-substituted or unsubstituted 3 to 15 membered heterocycloalkyl, R⁹-substituted or unsubstituted phenyl, or R⁹²-substituted or unsubstituted 5 to 6 membered heteroaryl.

In embodiments, R^{9′} is independently oxo, —O—P(O)(OR^{91A})₂, —OR^{91B}, —NR^{91B}R^{91B}, —NR^{91B}R^{91A}, —CO₂R^{91C}, —OCOR^{91B}, —CO₂R^{91B}, —SOR^{91B}, —SO₂R^{91C}, —CONR^{91B}R^{91A}, —SO₂NR^{91B}R^{91A}, —OCONR^{91B}R^{91A}, —NR^{91A}COR^{91B}, —NR^{91A}SOR^{91B}, —NR^{91A} CO₂R^{91B}, —COR^{91A}, -(unsubstituted C₁-C₄ saturated alkyl)-O—P(O)(OH)₂, -(unsubstituted C₁-C₄ saturated alkyl)-O—P(O)OR^{91A})₂, -(unsubstituted C₂-C₄ alkoxy)-O—P(O)(OH)₂, -(unsubstituted C₂-C₄alkoxy)-O— P(O)(OR^{91A})₂, (unsubstituted C₁-C₄ saturated alkyl)(unsubstituted C₁-C₄ saturated alkyl)amino, unsubstituted C₁-C₄ alkyl, halo-substituted (C₁-C₄ alkyl), hydroxy-substituted (C₁-C₄ saturated alkyl), halo-substituted (C₁-C₄ alkoxy), unsubstituted C₁-C₄ alkoxy, hydroxy-substituted (C₂-C₄ alkoxy), or unsubstituted C₁-C₄ alkoxy-(unsubstituted C₁-C₄ alkoxy).

In embodiments, R^{91B} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxyalkyl, R⁹²-substituted or unsubstituted phenyl, R⁹²-substituted or unsubstituted heteroaryl, or R⁹-substituted or unsubstituted heterocycloalkyl; wherein the R⁹²-substituted or unsubstituted phenyl, R⁹²-substituted or unsubstituted heteroaryl, and R⁹²-substituted or unsubstituted heterocycloalkyl are substituted with one or more R⁹² and R⁹² is independently halogen, —CN, —CF₃, unsubstituted C₁-C₄ saturated alkyl, halo(C₁-C₄ alkyl), unsubstituted C₂-C₄ alkenyl, unsubstituted C₂-C₄ alkynyl and unsubstituted C₃-C₆ cycloalkyl.

In embodiments, R^{91A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxyalkyl, C₁-C₁₀ aminoalkyl and C₁-C₁₀ thiolalkyl.

In embodiments, R^{91C} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxyalkyl, R⁹²-substituted or unsubstituted aryl, R⁹²-substituted or unsubstituted heteroaryl, or R⁹²-substituted or unsubstituted heterocycloalkyl; wherein the Rn-substituted aryl, R⁹²-substituted heteroaryl and R⁹²-substituted heterocycloalkyl are substituted with one or more R⁹² and R⁹² is independently halogen, —CN, —CF₃, or unsubstituted C₁-C₄ saturated alkyl.

In embodiments, R^{90A} and R^{90B} are independently hydrogen, R⁹¹-substituted or unsubstituted C₁-C₆ saturated alkyl; R^{90A} and R^{90B} substituents bonded to the same nitrogen atom may optionally be joined to form a R⁹¹-substituted or unsubstituted 5 to 6 membered heterocycloalkyl (e.g., morpholinyl, piperidinyl, piperazinyl, or pyrrolidinyl); wherein the R⁹¹-substituted C₁-C₆ saturated alkyl and R⁹¹-substituted or unsubstituted 5 to 6 membered heterocycloalkyl form by the joining of R^{90A} and R^{90B} substituents bonded to the same nitrogen atom (e.g., morpholinyl, piperidinyl, piperazinyl, or pyrrolidinyl) are substituted with 1 to 4 R⁹¹ and R⁹¹ is independently oxo, —O—P(O)(OR^{91A})₂, —OR^{91B}, —NR^{91B}R^{91B}, —NR^{91B}R^{91A}, —CO₂R^{91C}, —OCOR^{91B}, —CO₂R^{91B}, —SOR^{91B}, —SO₂R^{91C}, —CONR^{91B}R^{91A}, —SO₂NR^{91B}R^{91A}, —OCONR^{91B}R^{91A}, —NR^{91A}COR^{91B}, —NR^{91A}SOR^{91B}, —NR^{91A} CO₂R^{91B}, —COR^{91A}, -(unsubstituted C₁-C₄ saturated alkyl)-O—P(O)(OH)₂, -(unsubstituted C₁-C₄ saturated alkyl)-O—P(O)(OR^{91A})₂, -(unsubstituted C₂-C₄ alkoxy)-O—P(O)(OH)₂, -(unsubstituted C₂-C₄alkoxy)-O— P(O)(OR^{91A})₂, (unsubstituted C₁-C₄ saturated alkyl)(unsubstituted C₁-C₄ saturated alkyl)amino, unsubstituted C₁-C₄ alkyl, halo-substituted (C₁-C₄ alkyl), hydroxy-substituted (C₁-C₄ saturated alkyl), halo-substituted (C₁-C₄ alkoxy), unsubstituted C₁-C₄ alkoxy, hydroxy-substituted (C₂-C₄ alkoxy), C₁-C₄ alkoxy-(C₁-C₄ alkoxy); R^{91A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxyalkyl, C₁-C₁₀ aminoalkyl and C₁-C₁₀ thiolalkyl; R^{91B} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxyalkyl, R⁹²-substituted or unsubstituted phenyl, R⁹²-substituted or unsubstituted heteroaryl, or R⁹²-substituted or unsubstituted heterocycloalkyl and wherein said R⁹²-substituted or unsubstituted phenyl, R⁹²-substituted or unsubstituted heteroaryl, and R⁹²-substituted or unsubstituted heterocycloalkyl or R^{91B} are substituted with one or more R⁹² and R⁹² is independently halogen, —CN, —CF₃, unsubstituted C₁-C₄ saturated alkyl, halo(C₁-C₄ alkyl), unsubstituted C₂-C₄ alkenyl, unsubstituted C₂-C₄ alkynyl and unsubstituted C₃-C₆ cycloalkyl; R^{91C} is hydrogen, unsubstituted C₁-C₁₀ saturated alkyl, unsubstituted C₂-C₁₀ alkenyl, unsubstituted C₂-C₁₀ alkynyl, C₁-C₁₀ hydroxyalkyl, R⁹²-substituted or unsubstituted aryl, R⁹²-substituted or unsubstituted heteroaryl, or R⁹²-substituted or unsubstituted heterocycloalkyl, and wherein said R⁹²-substituted aryl, R⁹²-substituted heteroaryl and R⁹²-substituted heterocycloalkyl of R^{91C} are substituted with one or more R⁹² and R⁹² is independently halogen, —CN, —CF₃ and unsubstituted C₁-C₄ saturated alkyl. p is independently 2, 3, 4, 5, or 6.

In embodiments, the compound is a compound having formula (Ia-A):

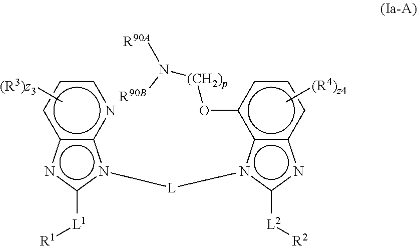

or a tautomer thereof or a salt thereof (e.g., a pharmaceutically acceptable salt) or a prodrug thereof. R¹, L¹, R², L², R³, z3, R⁴, z4, L, R^{90A}, and R^{90B} are as described herein, including in embodiments.

In embodiments, R^{90A} and R^{90B} are independently hydrogen, R⁹¹-substituted or unsubstituted C₁-C₆ saturated alkyl; R^{90A} and R^{90B} substituents bonded to the same nitrogen atom may optionally be joined to form a R⁹¹-substituted or unsubstituted 5 to 6 membered heterocycloalkyl (e.g., morpholinyl, piperidinyl, piperazinyl, or pyrrolidinyl); wherein the R⁹¹-substituted C₁-C₆ saturated alkyl and R⁹¹-substituted or unsubstituted 5 to 6 membered heterocycloalkyl form by the joining of R^{90A} and R^{90B} substituents bonded to the same nitrogen atom (e.g., morpholinyl, piperidinyl, piperazinyl, or pyrrolidinyl) are substituted with 1 to 4 R⁹¹ and R⁹¹ is independently oxo, —O—P(O)(OR^{91A})₂, —OR^{91B}, —NR^{91B}R^{91B}, —NR^{91B}R^{91A}, —CO₂R^{91C}, —OCOR^{91B}, —CO₂R^{91B}, —SOR^{91B}, —SO₂R^{91C}, —CONR^{91B}R^{91A}, —SO₂NR^{91B}R^{91A}, —OCON^{91B}R^{9A}, —NR^{9A}COR^{91B}, —NR^{91A}SOR^{91B}, —NR^{91A} CO₂R^{91B}, —COR^{91A}, -(unsubstituted C₁-C₄ saturated alkyl)-O—P(O)(OH)₂, -(unsubstituted C₁-C₄ saturated alkyl)-O—P(O)(OR^{91A})₂, -(unsubstituted C₂-C₄alkoxy)-O—P(O)(OH)₂, -(unsubstituted C₂-C₄alkoxy)-O— P(O)(OR^{91A})₂, (unsubstituted C₁-C₄ saturated alkyl)(unsubstituted C₁-C₄ saturated alkyl)amino, unsubstituted C₁-C₄alkyl, halo-substituted (C₁-C₄alkyl), hydroxy-substituted (C₁-C₄ saturated alkyl), halo-substituted (C₁-C₄alkoxy), unsubstituted C₁-C₄alkoxy, hydroxy-substituted (C₂-C₄ alkoxy), C₁-C₄alkoxy-(C₁-C₄alkoxy); R^{91A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxyalkyl, C₁-C₁₀ aminoalkyl and C₁-C₁₀ thiolalkyl; R^{91B} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxyalkyl, R⁹²-substituted or unsubstituted phenyl, R⁹²-substituted or unsubstituted heteroaryl, or R⁹²-substituted or unsubstituted heterocycloalkyl and wherein said R⁹²-substituted or unsubstituted phenyl, R⁹²-substituted or unsubstituted heteroaryl, and R⁹²-substituted or unsubstituted heterocycloalkyl or R^{91B} are substituted with one or more R⁹² and R⁹² is independently halogen, —CN, —CF₃, unsubstituted C₁-C₄ saturated alkyl, halo(C₁-C₄ alkyl), unsubstituted C₂-C₄ alkenyl, unsubstituted C₂-C₄ alkynyl and unsubstituted C₃-C₆ cycloalkyl; R^{91C} is hydrogen, unsubstituted C₁-C₁₀ saturated alkyl, unsubstituted C₂-C₁₀ alkenyl, unsubstituted C₂-C₁₀ alkynyl, C₁-C₁₀ hydroxyalkyl, R⁹²-substituted or unsubstituted aryl, R⁹²-substituted or unsubstituted heteroaryl, or R⁹²-substituted or unsubstituted heterocycloalkyl, and wherein said R⁹²-substituted aryl, R⁹²-substituted heteroaryl and R⁹²-substituted heterocycloalkyl of R^{91C} are substituted with one or more R⁹² and R⁹² is independently halogen, —CN, —CF₃ and unsubstituted C₁-C₄ saturated alkyl. p is independently 2, 3, 4, 5, or 6.

In embodiments, the compound is a compound having formula (Ib-A):

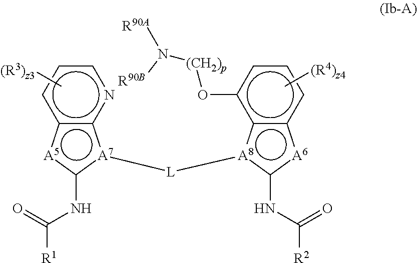

or a tautomer thereof or a salt thereof (e.g., a pharmaceutically acceptable salt) or a prodrug thereof. R¹, R², R³, z3, R⁴, z4, L, A⁵, A⁶, A⁷, A⁸, R^{90A}, and R^{90B} are as described herein, including in embodiments.

In embodiments, R^{90A} and R^{90B} are independently hydrogen, R⁹¹-substituted or unsubstituted C₁-C₆ saturated alkyl; R^{90A} and R^{90B} substituents bonded to the same nitrogen atom may optionally be joined to form a R⁹¹-substituted or unsubstituted 5 to 6 membered heterocycloalkyl (e.g., morpholinyl, piperidinyl, piperazinyl, or pyrrolidinyl); wherein the R⁹¹-substituted C₁-C₆ saturated alkyl and R⁹¹-substituted or unsubstituted 5 to 6 membered heterocycloalkyl form by the joining of R^{90A} and R^{90B} substituents bonded to the same nitrogen atom (e.g., morpholinyl, piperidinyl, piperazinyl, or pyrrolidinyl) are substituted with 1 to 4 R^{9′} and R⁹¹ is independently oxo, —O—P(O)(OR^{91A})₂, —OR^{91B}, —NR^{91B}R^{91B}, —NR^{9B}R^{91A}, —CO₂R^{91C}, —OCOR^{9B}, —CO₂R^{91B}, —SOR^{91B}, —SO₂R^{91C}, —CONR^{91B}R^{91A}, —SO₂NR^{91B}R^{91A}, —OCONR^{91B}R^{91A}, —NR^{91A}COR^{91B}, NR^{91A}SOR^{91B}, —NR^{91A} CO₂R^{91B}, —COR^{91A}, -(unsubstituted C₁-C₄ saturated alkyl)-O—P(O)(OH)₂, -(unsubstituted C₁-C₄ saturated alkyl)-O—P(O)(OR^{91A})₂, -(unsubstituted C₂-C₄ alkoxy)-O—P(O)(OH)₂, -(unsubstituted C₂-C₄alkoxy)-O— P(O)(OR^{91A})₂, (unsubstituted C₁-C₄ saturated alkyl)(unsubstituted C₁-C₄ saturated alkyl)amino, unsubstituted C₁-C₄ alkyl, halo-substituted (C₁-C₄ alkyl), hydroxy-substituted (C₁-C₄ saturated alkyl), halo-substituted (C₁-C₄ alkoxy), unsubstituted C₁-C₄ alkoxy, hydroxy-substituted (C₂-C₄ alkoxy), C₁-C₄ alkoxy-(C₁-C₄ alkoxy); R^{91A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxyalkyl, C₁-C₁₀ aminoalkyl and C₁-C₁₀ thiolalkyl; R^{91B} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxyalkyl, R⁹²-substituted or unsubstituted phenyl, R⁹²-substituted or unsubstituted heteroaryl, or R⁹²-substituted or unsubstituted heterocycloalkyl and wherein said R⁹²-substituted or unsubstituted phenyl, R⁹²-substituted or unsubstituted heteroaryl, and R⁹²-substituted or unsubstituted heterocycloalkyl or R^{91B} are substituted with one or more R⁹² and R⁹² is independently halogen, —CN, —CF₃, unsubstituted C₁-C₄ saturated alkyl, halo(C₁-C₄ alkyl), unsubstituted C₂-C₄ alkenyl, unsubstituted C₂-C₄ alkynyl and unsubstituted C₃-C₆ cycloalkyl; R^{91C} is hydrogen, unsubstituted C₁-C₁₀ saturated alkyl, unsubstituted C₂-C₁₀ alkenyl, unsubstituted C₂-C₁₀ alkynyl, C₁-C₁₀ hydroxyalkyl, R⁹²-substituted or unsubstituted aryl, R⁹²-substituted or unsubstituted heteroaryl, or R⁹²-substituted or unsubstituted heterocycloalkyl, and wherein said R⁹²-substituted aryl, R⁹²-substituted heteroaryl and R⁹²-substituted heterocycloalkyl of R^{91C} are substituted with one or more R⁹² and R⁹² is independently halogen, —CN, —CF₃ and unsubstituted C₁-C₄ saturated alkyl. p is independently 2, 3, 4, 5, or 6.

In embodiments, the compound is a compound having formula (Ic-A):

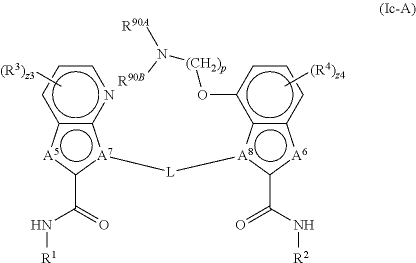

or a tautomer thereof or a salt thereof (e.g., a pharmaceutically acceptable salt) or a prodrug thereof. R¹, R², R³, z3, R⁴, z4, L, A⁵, A⁶, A⁷, A⁸, R^{90A}, and R^{90B} are as described herein, including in embodiments.

In embodiments, R^{90A} and R^{90B} are independently hydrogen, R⁹¹-substituted or unsubstituted C₁-C₆ saturated alkyl; R^{90A} and R^{90B} substituents bonded to the same nitrogen atom may optionally be joined to form a R⁹¹-substituted or unsubstituted 5 to 6 membered heterocycloalkyl (e.g., morpholinyl, piperidinyl, piperazinyl, or pyrrolidinyl); wherein the R⁹¹-substituted C₁-C₆ saturated alkyl and R⁹¹-substituted or unsubstituted 5 to 6 membered heterocycloalkyl form by the joining of R^{90A} and R^{90B} substituents bonded to the same nitrogen atom (e.g., morpholinyl, piperidinyl, piperazinyl, or pyrrolidinyl) are substituted with 1 to 4 R⁹¹ and R⁹¹ is independently oxo, —O—P(O)(OR^{91A})₂, —OR^{91B}, —NR^{91B}R^{91B}, —NR^{91B}R^{91A}, —CO₂R^{91C}, —OCOR^{91B}, —CO₂R^{91B}, —SOR^{91B}, —SO₂R^{91C}, —CONR^{91B}R^{91A}, —SO₂NR^{91B}R^{91A}, —OCONR^{91B}R^{91A}, —NR^{91A}COR^{9B}, —NR^{91A}SOR^{91B}, —NR^{91A} CO₂R^{91B}, —COR^{91A}, -(unsubstituted C₁-C₄ saturated alkyl)-O—P(O)(OH)₂, -(unsubstituted C₁-C₄ saturated alkyl)-O—P(O)(OR^{91A})₂, -(unsubstituted C₂-C₄ alkoxy)-O—P(O)(OH)₂, -(unsubstituted C₂-C₄alkoxy)-O— P(O)(OR^{91A})₂, (unsubstituted C₁-C₄ saturated alkyl)(unsubstituted C₁-C₄ saturated alkyl)amino, unsubstituted C₁-C₄ alkyl, halo-substituted (C₁-C₄ alkyl), hydroxy-substituted (C₁-C₄ saturated alkyl), halo-substituted (C₁-C₄ alkoxy), unsubstituted C₁-C₄ alkoxy, hydroxy-substituted (C₂-C₄ alkoxy), C₁-C₄ alkoxy-(C₁-C₄ alkoxy); R^{91A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxyalkyl, C₁-C₁₀ aminoalkyl and C₁-C₁₀ thiolalkyl; R^{91B} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxyalkyl, R⁹²-substituted or unsubstituted phenyl, R⁹²-substituted or unsubstituted heteroaryl, or R⁹²-substituted or unsubstituted heterocycloalkyl and wherein said R⁹²-substituted or unsubstituted phenyl, R⁹²-substituted or unsubstituted heteroaryl, and R⁹²-substituted or unsubstituted heterocycloalkyl or R^{91B} are substituted with one or more R⁹² and R⁹² is independently halogen, —CN, —CF₃, unsubstituted C₁-C₄ saturated alkyl, halo(C₁-C₄ alkyl), unsubstituted C₂-C₄ alkenyl, unsubstituted C₂-C₄ alkynyl and unsubstituted C₃-C₆ cycloalkyl; R^{91C} is hydrogen, unsubstituted C₁-C₁₀ saturated alkyl, unsubstituted C₂-C₁₀ alkenyl, unsubstituted C₂-C₁₀ alkynyl, C₁-C₁₀ hydroxyalkyl, R⁹²-substituted or unsubstituted aryl, R⁹²-substituted or unsubstituted heteroaryl, or R⁹²-substituted or unsubstituted heterocycloalkyl, and wherein said R⁹²-substituted aryl, R⁹²-substituted heteroaryl and R⁹²-substituted heterocycloalkyl of R^{91C} are substituted with one or more R⁹² and R⁹² is independently halogen, —CN, —CF₃ and unsubstituted C₁-C₄ saturated alkyl. p is independently 2, 3, 4, 5, or 6.

In embodiments, the compound is a compound having formula (Id-A):

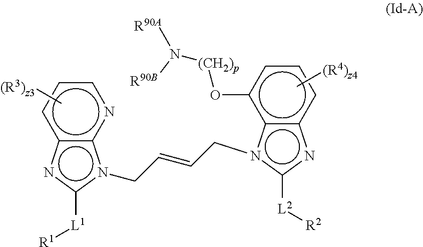

or a tautomer thereof or a salt thereof (e.g., a pharmaceutically acceptable salt) or a prodrug thereof. R¹, L¹, R², L², R³, z3, R⁴, z4, R^{90A}, and R^{90B} are as described herein, including in embodiments.

In embodiments, R^{90A} and R^{90B} are independently hydrogen, R⁹¹-substituted or unsubstituted C₁-C₆ saturated alkyl; R^{90A} and R^{90B} substituents bonded to the same nitrogen atom may optionally be joined to form a R⁹¹-substituted or unsubstituted 5 to 6 membered heterocycloalkyl (e.g., morpholinyl, piperidinyl, piperazinyl, or pyrrolidinyl); wherein the R⁹¹-substituted C₁-C₆ saturated alkyl and R⁹¹-substituted or unsubstituted 5 to 6 membered heterocycloalkyl form by the joining of R^{90A} and R^{90B} substituents bonded to the same nitrogen atom (e.g., morpholinyl, piperidinyl, piperazinyl, or pyrrolidinyl) are substituted with 1 to 4 R⁹¹ and R⁹¹ is independently oxo, —O—P(O)(OR^{9A})₂, —OR^{91B}, —NR^{91B}R^{91B}, —NR^{91B}R^{91A}, —CO₂R^{91C}, —OCOR^{91B}, —CO₂R^{91B}, —SOR^{91B}, —SO₂R^{91C}, —CONR^{91B}R^{91A}, —SO₂NR^{91B}R^{91A}, —OCONR^{91B}R^{91A}, —NR^{91A}COR^{91B}, —R^{91A}SOR^{91B}, —NR^{91A} CO₂R^{91B}, —COR^{91A}, -(unsubstituted C₁-C₄ saturated alkyl)-O—P(O)(OH)₂, -(unsubstituted C₁-C₄ saturated alkyl)-O—P(O)(OR^{91A})₂, -(unsubstituted C₂-C₄ alkoxy)-O—P(O)(OH)₂, -(unsubstituted C₂-C₄alkoxy)-O— P(O)(OR^{91A})₂, (unsubstituted C₁-C₄ saturated alkyl)(unsubstituted C₁-C₄ saturated alkyl)amino, unsubstituted C₁-C₄ alkyl, halo-substituted (C₁-C₄ alkyl), hydroxy-substituted (C₁-C₄ saturated alkyl), halo-substituted (C₁-C₄ alkoxy), unsubstituted C₁-C₄ alkoxy, hydroxy-substituted (C₂-C₄ alkoxy), C₁-C₄ alkoxy-(C₁-C₄ alkoxy); R^{91A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxyalkyl, C₁-C₁₀ aminoalkyl and C₁-C₁₀ thiolalkyl; R^{91B} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxyalkyl, R⁹²-substituted or unsubstituted phenyl, R⁹²-substituted or unsubstituted heteroaryl, or R⁹²-substituted or unsubstituted heterocycloalkyl and wherein said R⁹²-substituted or unsubstituted phenyl, R⁹²-substituted or unsubstituted heteroaryl, and R⁹²-substituted or unsubstituted heterocycloalkyl or R^{91B} are substituted with one or more R⁹² and R⁹² is independently halogen, —CN, —CF₃, unsubstituted C₁-C₄ saturated alkyl, halo(C₁-C₄ alkyl), unsubstituted C₂-C₄ alkenyl, unsubstituted C₂-C₄ alkynyl and unsubstituted C₃-C₆ cycloalkyl; R^{91C} is hydrogen, unsubstituted C₁-C₁₀ saturated alkyl, unsubstituted C₂-C₁₀ alkenyl, unsubstituted C₂-C₁₀ alkynyl, C₁-C₁₀ hydroxyalkyl, R⁹²-substituted or unsubstituted aryl, R⁹²-substituted or unsubstituted heteroaryl, or R⁹²-substituted or unsubstituted heterocycloalkyl, and wherein said R⁹²-substituted aryl, R⁹²-substituted heteroaryl and R⁹²-substituted heterocycloalkyl of R^{91C} are substituted with one or more R⁹² and R⁹² is independently halogen, —CN, —CF₃ and unsubstituted C₁-C₄ saturated alkyl. p is independently 2, 3, 4, 5, or 6.

In embodiments, the compound is a compound having formula (Ie-A):

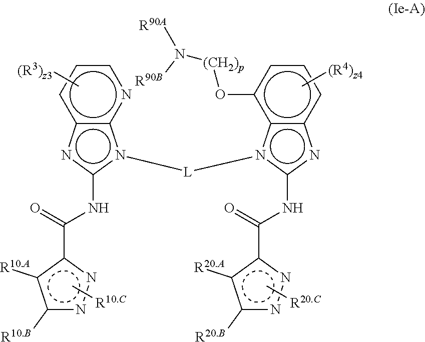

or a tautomer thereof or a salt thereof (e.g., a pharmaceutically acceptable salt) or a prodrug thereof. R^{10.A}, R^{10.B}, R^{10.C}, R^{20.A}, R^{20.B}, R^{20.C}, L, R³, z3, R⁴, z4, R^{90A}, and R^{90B} are as described herein, including in embodiments.

In embodiments, R^{90A} and R^{90B} are independently hydrogen, R⁹¹-substituted or unsubstituted C₁-C₆ saturated alkyl; R^{90A} and R^{90B} substituents bonded to the same nitrogen atom may optionally be joined to form a R⁹¹-substituted or unsubstituted 5 to 6 membered heterocycloalkyl (e.g., morpholinyl, piperidinyl, piperazinyl, or pyrrolidinyl); wherein the R⁹¹-substituted C₁-C₆ saturated alkyl and R⁹¹-substituted or unsubstituted 5 to 6 membered heterocycloalkyl form by the joining of R^{90A} and R^{90B} substituents bonded to the same nitrogen atom (e.g., morpholinyl, piperidinyl, piperazinyl, or pyrrolidinyl) are substituted with 1 to 4 R⁹¹ and R⁹¹ is independently oxo, —O—P(O)(OR^{9A})₂, —OR^{91B}, —NR^{91B}R^{91B}, —NR^{91B}R^{91A}, —CO₂R^{91C}, —OCPR^{91B}, —CO₂R^{91B}, —SOR^{91B}, —SO₂R^{91C}, —CONR^{91B}R^{91A}, —SO₂NR^{91B}R^{91A}, —OCONR^{91B}R^{91A}, —NR^{91A}COR^{9B}, —NR^{9A}SOR^{91B}, —NR^{91A} CO₂R^{91B}, —COR^{91A}, -(unsubstituted C₁-C₄ saturated alkyl)-O—P(O)(OH)₂, -(unsubstituted C₁-C₄ saturated alkyl)-O—P(O)(OR^{91A})₂, -(unsubstituted C₂-C₄ alkoxy)-O—P(O)(OH)₂, -(unsubstituted C₂-C₄alkoxy)-O— P(O)(OR^{91A})₂, (unsubstituted C₁-C₄ saturated alkyl)(unsubstituted C₁-C₄ saturated alkyl)amino, unsubstituted C₁-C₄ alkyl, halo-substituted (C₁-C₄ alkyl), hydroxy-substituted (C₁-C₄ saturated alkyl), halo-substituted (C₁-C₄ alkoxy), unsubstituted C₁-C₄ alkoxy, hydroxy-substituted (C₂-C₄ alkoxy), C₁-C₄ alkoxy-(C₁-C₄ alkoxy); R^{91A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxyalkyl, C₁-C₁₀ aminoalkyl and C₁-C₁₀ thiolalkyl; R^{91B} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxyalkyl, R⁹²-substituted or unsubstituted phenyl, R⁹²-substituted or unsubstituted heteroaryl, or R⁹²-substituted or unsubstituted heterocycloalkyl and wherein said R⁹²-substituted or unsubstituted phenyl, R⁹²-substituted or unsubstituted heteroaryl, and R⁹²-substituted or unsubstituted heterocycloalkyl or R^{91B} are substituted with one or more R⁹² and R⁹² is independently halogen, —CN, —CF₃, unsubstituted C₁-C₄ saturated alkyl, halo(C₁-C₄ alkyl), unsubstituted C₂-C₄ alkenyl, unsubstituted C₂-C₄ alkynyl and unsubstituted C₃-C₆ cycloalkyl; R^{91C} is hydrogen, unsubstituted C₁-C₁₀ saturated alkyl, unsubstituted C₂-C₁₀ alkenyl, unsubstituted C₂-C₁₀ alkynyl, C₁-C₁₀ hydroxyalkyl, R⁹²-substituted or unsubstituted aryl, R⁹²-substituted or unsubstituted heteroaryl, or R⁹²-substituted or unsubstituted heterocycloalkyl, and wherein said R⁹²-substituted aryl, R⁹²-substituted heteroaryl and R⁹²-substituted heterocycloalkyl of R^{91C} are substituted with one or more R⁹² and R⁹² is independently halogen, —CN, —CF₃ and unsubstituted C₁-C₄ saturated alkyl. p is independently 2, 3, 4, 5, or 6.

In embodiments, (e.g., the compound of Formula (Ia-A), (Ib-A), (Ic-A), (Id-A) or (Ie-A)), R³ and R⁴ are hydrogen.

In embodiments, (e.g., the compound of Formula (Ia-A), (Ib-A), (Ic-A), (Id-A) or (le-A)), R³ and R⁴ are —CONH₂, z3=1 and z4=1.

In embodiments, the compound is a compound having formula (Ie-A-A):

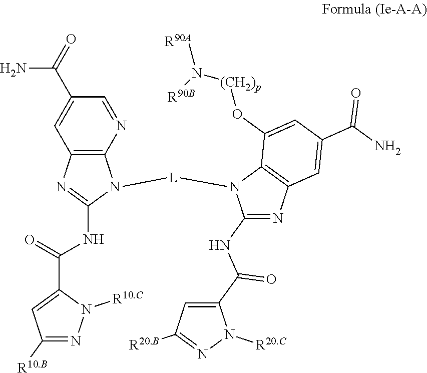

or a tautomer thereof or a salt thereof (e.g., a pharmaceutically acceptable salt) or a prodrug thereof. R^{10.B}, R^{10.C}, R^{20.B}, R^{20.C}, L, R^{90A}, and R^{90B} are as described herein, including in embodiments.

In embodiments, R^{90A} and R^{90B} are independently hydrogen, R⁹¹-substituted or unsubstituted C₁-C₆ saturated alkyl; R^{90A} and R^{90B} substituents bonded to the same nitrogen atom may optionally be joined to form a R⁹¹-substituted or unsubstituted 5 to 6 membered heterocycloalkyl (e.g., morpholinyl, piperidinyl, piperazinyl, or pyrrolidinyl); wherein the R⁹¹-substituted C₁-C₆ saturated alkyl and R⁹¹-substituted or unsubstituted 5 to 6 membered heterocycloalkyl form by the joining of R^{90A} and R^{90B} substituents bonded to the same nitrogen atom (e.g., morpholinyl, piperidinyl, piperazinyl, or pyrrolidinyl) are substituted with 1 to 4 R⁹¹ and R⁹¹ is independently oxo, —O—P(O)(OR^{91A})₂, —OR^{91B}, —NR^{91B}R^{91B}, —NR^{91B}R^{9A}, —CO₂R^{91C}, —OCOR^{91B}, —CO₂R^{91B}, —SOR^{91B}, —SO₂R^{91C}, —CONR^{91B}R^{91A}, —SO₂NR^{91B}R^{91A}, —OCONR^{91B}R^{91A}, —NR^{91A}COR^{91B}, —NR^{91A}SOR^{91B}, —NR^{91A} CO₂R^{91B}, —COR^{91A}, -(unsubstituted C₁-C₄ saturated alkyl)-O—P(O)(OH)₂, -(unsubstituted C₁-C₄ saturated alkyl)-O—P(O)(OR^{91A})₂, -(unsubstituted C₂-C₄ alkoxy)-O—P(O)(OH)₂, -(unsubstituted C₂-C₄alkoxy)-O— P(O)(OR^{91A})₂, (unsubstituted C₁-C₄ saturated alkyl)(unsubstituted C₁-C₄ saturated alkyl)amino, unsubstituted C₁-C₄ alkyl, halo-substituted (C₁-C₄ alkyl), hydroxy-substituted (C₁-C₄ saturated alkyl), halo-substituted (C₁-C₄ alkoxy), unsubstituted C₁-C₄ alkoxy, hydroxy-substituted (C₂-C₄ alkoxy), C₁-C₄ alkoxy-(C₁-C₄ alkoxy); R^{91A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxyalkyl, C₁-C₁₀ aminoalkyl and C₁-C₁₀ thiolalkyl; R^{91B} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxyalkyl, R⁹²-substituted or unsubstituted phenyl, R⁹²-substituted or unsubstituted heteroaryl, or R⁹²-substituted or unsubstituted heterocycloalkyl and wherein said R⁹²-substituted or unsubstituted phenyl, R⁹²-substituted or unsubstituted heteroaryl, and R⁹²-substituted or unsubstituted heterocycloalkyl or R^{91B} are substituted with one or more R⁹² and R⁹² is independently halogen, —CN, —CF₃, unsubstituted C₁-C₄ saturated alkyl, halo(C₁-C₄ alkyl), unsubstituted C₂-C₄ alkenyl, unsubstituted C₂-C₄ alkynyl and unsubstituted C₃-C₆ cycloalkyl, R^{91C} is hydrogen, unsubstituted C₁-C₁₀ saturated alkyl, unsubstituted C₂-C₁₀ alkenyl, unsubstituted C₂-C₁₀ alkynyl, C₁-C₁₀ hydroxyalkyl, R⁹²-substituted or unsubstituted aryl, R⁹²-substituted or unsubstituted heteroaryl, or R⁹²-substituted or unsubstituted heterocycloalkyl, and wherein said R⁹²-substituted aryl, R⁹²-substituted heteroaryl and R⁹²-substituted heterocycloalkyl of R^{91C} are substituted with one or more R⁹² and R⁹² is independently halogen, —CN, —CF₃ and unsubstituted C₁-C₄ saturated alkyl. p is independently 2, 3, 4, 5, or 6.

In embodiments, (e.g., the compound of Formula (Ia-A), (Ib-A), (Ic-A), (Id-A) or (Ie-A)), R³ and R⁴ are hydrogen.

In embodiments, the compound is a compound having formula (Ie-A-B):

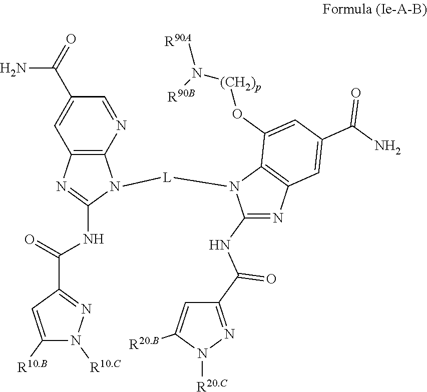

or a tautomer thereof or a salt thereof (e.g., a pharmaceutically acceptable salt) or a prodrug thereof. R^{10.B}, R^{10.C}, R^{20.B}, R^{20.C}, L, R^{90A}, and R^{90B} are as described herein, including in embodiments.

In embodiments, R^{90A} and R^{90B} are independently hydrogen, R⁹¹-substituted or unsubstituted C₁-C₆ saturated alkyl; R^{90A} and R^{90B} substituents bonded to the same nitrogen atom may optionally be joined to form a R⁹¹-substituted or unsubstituted 5 to 6 membered heterocycloalkyl (e.g., morpholinyl, piperidinyl, piperazinyl, or pyrrolidinyl); wherein the R⁹¹-substituted C₁-C₆ saturated alkyl and R⁹¹-substituted or unsubstituted 5 to 6 membered heterocycloalkyl form by the joining of R^{90A} and R^{9B} substituents bonded to the same nitrogen atom (e.g., morpholinyl, piperidinyl, piperazinyl, or pyrrolidinyl) are substituted with 1 to 4 R⁹¹ and R⁹¹ is independently oxo, —O—P(O)(OR^{91A})₂, —OR^{91B}, —NR^{91B}R^{91B}, —NR^{91B}R^{91A}, —CO₂R^{91C}, —OCOR^{91B}, —CO₂R^{91B}, —SOR^{91B}, —SO₂R^{91C}, —CONR^{91B}R^{91A}, —SO₂NR^{91B}R^{91A}, —OCONR^{91B}R^{91A}, —NR^{91A}COR^{91B}, —NR^{91A}SOR^{91B}, —NR^{91A} CO₂R^{91B}, —COR^{91A}, -(unsubstituted C₁-C₄ saturated alkyl)-O—P(O)(OH)₂, -(unsubstituted C₁-C₄ saturated alkyl)-O—P(O)(OR^{91A})₂, -(unsubstituted C₂-C₄ alkoxy)-O—P(O)(OH)₂, -(unsubstituted C₂-C₄alkoxy)-O— P(O)(OR^{91A})₂, (unsubstituted C₁-C₄ saturated alkyl)(unsubstituted C₁-C₄ saturated alkyl)amino, unsubstituted C₁-C₄ alkyl, halo-substituted (C₁-C₄ alkyl), hydroxy-substituted (C₁-C₄ saturated alkyl), halo-substituted (C₁-C₄ alkoxy), unsubstituted C₁-C₄ alkoxy, hydroxy-substituted (C₂-C₄ alkoxy), C₁-C₄ alkoxy-(C₁-C₄ alkoxy); R^{91A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxyalkyl, C₁-C₁₀ aminoalkyl and C₁-C₁₀ thiolalkyl; R^{91B} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxyalkyl, R⁹²-substituted or unsubstituted phenyl, R⁹²-substituted or unsubstituted heteroaryl, or R⁹²-substituted or unsubstituted heterocycloalkyl and wherein said R⁹²-substituted or unsubstituted phenyl, R⁹²-substituted or unsubstituted heteroaryl, and R⁹²-substituted or unsubstituted heterocycloalkyl or R^{91B} are substituted with one or more R⁹² and R⁹² is independently halogen, —CN, —CF₃, unsubstituted C₁-C₄ saturated alkyl, halo(C₁-C₄ alkyl), unsubstituted C₂-C₄ alkenyl, unsubstituted C₂-C₄ alkynyl and unsubstituted C₃-C₆ cycloalkyl; R^{91C} is hydrogen, unsubstituted C₁-C₁₀ saturated alkyl, unsubstituted C₂-C₁₀ alkenyl, unsubstituted C₂-C₁₀ alkynyl, C₁-C₁₀ hydroxyalkyl, R⁹²-substituted or unsubstituted aryl, R⁹²-substituted or unsubstituted heteroaryl, or R⁹²-substituted or unsubstituted heterocycloalkyl, and wherein said R⁹²-substituted aryl, R⁹²-substituted heteroaryl and R⁹²-substituted heterocycloalkyl of R^{91C} are substituted with one or more R⁹² and R⁹² is independently halogen, —CN, —CF₃ and unsubstituted C₁-C₄ saturated alkyl. p is independently 2, 3, 4, 5, or 6.

In embodiments, (e.g., the compound of Formula (Ie-A-A) or (Ie-A-B)), wherein R^{10.B}, and R^{20.B} are independently unsubstituted C₁-C₄ saturated alkyl, unsubstituted C₂-C₄ alkenyl; R^{10.C} and R^{10.C} are independently C₁-C₆ alkyl; L is unsubstituted —C₂-C₁₀ alkenyl-.

In embodiments, (e.g., the compound of Formula (Ie-A-A) or (Ie-A-B)), wherein R^{10.B}, and R^{20.B} are independently unsubstituted C₁-C₄ saturated alkyl.

In embodiments, R^{10.C} and R^{10.C} are independently unsubstituted C₁-C₆ saturated alkyl; L is unsubstituted —C₂-C₁₀ alkenyl-.

In embodiments, R¹ is independently substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted R¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹ is substituted with a plurality of groups selected from substituent groups, size-limited substituent groups, and lower substituent groups; each substituent group, size-limited substituent group, and/or lower substituent group may optionally be different. In embodiments, when R¹ is substituted, it is substituted with at least one substituent group. In embodiments, when R¹ is substituted, it is substituted with at least one size-limited substituent group. In embodiments, when R¹ is substituted, it is substituted with at least one lower substituent group.

In embodiments, R² is independently substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted R² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R² is substituted with a plurality of groups selected from substituent groups, size-limited substituent groups, and lower substituent groups; each substituent group, size-limited substituent group, and/or lower substituent group may optionally be different. In embodiments, when R² is substituted, it is substituted with at least one substituent group. In embodiments, when R² is substituted, it is substituted with at least one size-limited substituent group. In embodiments, when R² is substituted, it is substituted with at least one lower substituent group.

In embodiments, R³ is independently halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O) NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, —O—P(O)(OH)₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), or substituted or unsubstituted cycloalkyl (e.g., C₃-C₈, C₃-C₆, C₄-C₆, or C₅-C₆).

In embodiments, a substituted R³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R³ is substituted with a plurality of groups selected from substituent groups, size-limited substituent groups, and lower substituent groups; each substituent group, size-limited substituent group, and/or lower substituent group may optionally be different. In embodiments, when R³ is substituted, it is substituted with at least one substituent group. In embodiments, when R³ is substituted, it is substituted with at least one size-limited substituent group. In embodiments, when R³ is substituted, it is substituted with at least one lower substituent group.

In embodiments, R^{3A} is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted R^{3A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3A} is substituted with a plurality of groups selected from substituent groups, size-limited substituent groups, and lower substituent groups; each substituent group, size-limited substituent group, and/or lower substituent group may optionally be different. In embodiments, when R^{3A} is substituted, it is substituted with at least one substituent group. In embodiments, when R^{3A} is substituted, it is substituted with at least one size-limited substituent group. In embodiments, when R^{3A} is substituted, it is substituted with at least one lower substituent group.

In embodiments, R^{3B} is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted R^{3B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3B} is substituted with a plurality of groups selected from substituent groups, size-limited substituent groups, and lower substituent groups; each substituent group, size-limited substituent group, and/or lower substituent group may optionally be different. In embodiments, when R^{3B} is substituted, it is substituted with at least one substituent group. In embodiments, when R^{3B} is substituted, it is substituted with at least one size-limited substituent group. In embodiments, when R^{3B} is substituted, it is substituted with at least one lower substituent group.

In embodiments, a substituted ring formed when R^{3A} and R^{3B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{3A} and R^{3B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{3A} and R^{3B} substituents bonded to the same nitrogen atom are joined is substituted, it is substituted with at least one substituent group. In embodiments, when the substituted ring formed when R^{3A} and R^{3B} substituents bonded to the same nitrogen atom are joined is substituted, it is substituted with at least one size-limited substituent group. In embodiments, when the substituted ring formed when R^{3A} and R^{3B} substituents bonded to the same nitrogen atom are joined is substituted, it is substituted with at least one lower substituent group.

In embodiments, R^{3C} is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted R^{3C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3C} is substituted with a plurality of groups selected from substituent groups, size-limited substituent groups, and lower substituent groups; each substituent group, size-limited substituent group, and/or lower substituent group may optionally be different. In embodiments, when R^{3C} is substituted, it is substituted with at least one substituent group. In embodiments, when R^{3C} is substituted, it is substituted with at least one size-limited substituent group. In embodiments, when R^{3C} is substituted, it is substituted with at least one lower substituent group.

In embodiments, R^{3D} is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted R^{3D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3D} is substituted with a plurality of groups selected from substituent groups, size-limited substituent groups, and lower substituent groups; each substituent group, size-limited substituent group, and/or lower substituent group may optionally be different. In embodiments, when R^{3D} is substituted, it is substituted with at least one substituent group. In embodiments, when R^{3D} is substituted, it is substituted with at least one size-limited substituent group. In embodiments, when R^{3D} is substituted, it is substituted with at least one lower substituent group.

In embodiments, R⁴ is independently halogen, —CX⁴₃, —CHX⁴₂, —CH₂X⁴, —OCX⁴₃, —OCH₂X⁴, —OCHX⁴₂, —CN, —SO₄R^{4D}, —SOᵥ₄NR^{4A}R^{4B}, —N^{4C}NR^{4A}R^{4B}, —ONR^{4A}R^{4B}, —NHC(O)NR^{4C}NR^{4A}R^{4B}, —NHC(O)NR^{4A}R^{4B}, —N(O)ₘ₄, —N^{4A}R^{4B}, C(O)R^{4C}, —C(O)—OR^{4C}, —C(O) N^{4A}R^{4B}, —OR^{4D}, —NR^{4A}SO₂R^{4D}, —NR^{4A}C(O)R^{4C}, —NR^{4A}C(O)ORC, —NR^{4A}OR^{4C}, —SF₅, —N₃, —O—P(O)(OH)₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆).

In embodiments, a substituted R⁴ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁴ is substituted with a plurality of groups selected from substituent groups, size-limited substituent groups, and lower substituent groups; each substituent group, size-limited substituent group, and/or lower substituent group may optionally be different. In embodiments, when R⁴ is substituted, it is substituted with at least one substituent group. In embodiments, when R⁴ is substituted, it is substituted with at least one size-limited substituent group. In embodiments, when R⁴ is substituted, it is substituted with at least one lower substituent group.

In embodiments, R^{4A} is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted R^{4A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4A} is substituted with a plurality of groups selected from substituent groups, size-limited substituent groups, and lower substituent groups; each substituent group, size-limited substituent group, and/or lower substituent group may optionally be different. In embodiments, when R^{4A} is substituted, it is substituted with at least one substituent group. In embodiments, when R^{4A} is substituted, it is substituted with at least one size-limited substituent group. In embodiments, when R^{4A} is substituted, it is substituted with at least one lower substituent group.

In embodiments, R^{4B} is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted R^{4B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4B} is substituted with a plurality of groups selected from substituent groups, size-limited substituent groups, and lower substituent groups; each substituent group, size-limited substituent group, and/or lower substituent group may optionally be different. In embodiments, when R^{4B} is substituted, it is substituted with at least one substituent group. In embodiments, when R^{4B} is substituted, it is substituted with at least one size-limited substituent group. In embodiments, when R^{4B} is substituted, it is substituted with at least one lower substituent group.

In embodiments, a substituted ring formed when R^{4A} and R^{4B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{4A} and R^{4B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{4A} and R^{4B} substituents bonded to the same nitrogen atom are joined is substituted, it is substituted with at least one substituent group. In embodiments, when the substituted ring formed when R^{4A} and R^{4B} substituents bonded to the same nitrogen atom are joined is substituted, it is substituted with at least one size-limited substituent group. In embodiments, when the substituted ring formed when R^{4A} and R^{4B} substituents bonded to the same nitrogen atom are joined is substituted, it is substituted with at least one lower substituent group.

In embodiments, R^{4C} is independently hydrogen, —CCl₃, —CBr₃, —CF₃, —CI₃, —CHCl₂, —CHBr₂, —CHF₂, —CHI₂, -CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted R^{4C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4C} is substituted with a plurality of groups selected from substituent groups, size-limited substituent groups, and lower substituent groups; each substituent group, size-limited substituent group, and/or lower substituent group may optionally be different. In embodiments, when R^{4C} is substituted, it is substituted with at least one substituent group. In embodiments, when R^{4C} is substituted, it is substituted with at least one size-limited substituent group. In embodiments, when R^{4C} is substituted, it is substituted with at least one lower substituent group.

In embodiments, R^{4D} is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted R^{4D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4D} is substituted with a plurality of groups selected from substituent groups, size-limited substituent groups, and lower substituent groups; each substituent group, size-limited substituent group, and/or lower substituent group may optionally be different. In embodiments, when R^{4D} is substituted, it is substituted with at least one substituent group. In embodiments, when R^{4D} is substituted, it is substituted with at least one size-limited substituent group. In embodiments, when R^{4D} is substituted, it is substituted with at least one lower substituent group.

In embodiments, L³ is independently a bond, substituted or unsubstituted alkylene (e.g., C₁-C₁₀, C₁-C₈, C₁-C₆, C₁-C₄, C₁-C₂, C₂-C₁₀, C₂-C₈, C₂-C₆, or C₂-C₄,), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₁₀, C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

In embodiments, a substituted L³ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L³ is substituted with a plurality of groups selected from substituent groups, size-limited substituent groups, and lower substituent groups; each substituent group, size-limited substituent group, and/or lower substituent group may optionally be different. In embodiments, when L³ is substituted, it is substituted with at least one substituent group. In embodiments, when L³ is substituted, it is substituted with at least one size-limited substituent group. In embodiments, when L³ is substituted, it is substituted with at least one lower substituent group.

In embodiments, L⁵ is independently a bond, substituted or unsubstituted alkylene (e.g., C₁-C₁₀, C₁-C₈, C₁-C₆, C₁-C₄, C₁-C₂, C₂-C₁₀, C₂-C₈, C₂-C₆, or C₂-C₄,), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₁₀, C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

In embodiments, a substituted L⁵ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L⁵ is substituted with a plurality of groups selected from substituent groups, size-limited substituent groups, and lower substituent groups; each substituent group, size-limited substituent group, and/or lower substituent group may optionally be different. In embodiments, when L⁵ is substituted, it is substituted with at least one substituent group. In embodiments, when L⁵ is substituted, it is substituted with at least one size-limited substituent group. In embodiments, when L⁵ is substituted, it is substituted with at least one lower substituent group.

In embodiments, L⁴ is independently a bond, —N(R^{L4})—, —O—, —S—, —SO₂—, —C(O)—, —C(O)N(R^{L4})—, —N(R^{L4})C(O)—, —N(R^{L4})C(O)NH—, —NHC(O)N(R^{L4})—, —C(O)O—, —OC(O)—, —SO₂N(R^{L4})—, —N(R^{L4})SO₂—, substituted or unsubstituted alkylene (e.g., C₁-C₁₀, C₁-C₈, C₁-C₆, C₁-C₄, C₁-C₂, C₂-C₁₀, C₂-C₈, C₂-C₆, or C₂-C₄,), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₁₀, C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

In embodiments, a substituted L⁴ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L⁴ is substituted with a plurality of groups selected from substituent groups, size-limited substituent groups, and lower substituent groups; each substituent group, size-limited substituent group, and/or lower substituent group may optionally be different. In embodiments, when L⁴ is substituted, it is substituted with at least one substituent group. In embodiments, when L⁴ is substituted, it is substituted with at least one size-limited substituent group. In embodiments, when L⁴ is substituted, it is substituted with at least one lower substituent group.

In embodiments, R^{L4} is independently a bond, —N(R^{L4})—, —O—, —S—, —SO₂—, —C(O)—, —C(O)N(R^{L4})—, —N(R^{L4})C(O)—, —N(R^{L4})C(O)NH—, —NHC(O)N(R^{L4})—, —C(O)O—, —OC(O)—, —SO₂N(R^{L4})—, —N(R^{L4})SO₂—, substituted or unsubstituted alkylene (e.g., C₁-C₁₀, C₁-C₈, C₁-C₆, C₁-C₄, C₁-C₂, C₂-C₁₀, C₂-C₈, C₂-C₆, or C₂-C₄,), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₁₀, C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

In embodiments, a substituted L⁴ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L⁴ is substituted with a plurality of groups selected from substituent groups, size-limited substituent groups, and lower substituent groups; each substituent group, size-limited substituent group, and/or lower substituent group may optionally be different. In embodiments, when L⁴ is substituted, it is substituted with at least one substituent group. In embodiments, when L⁴ is substituted, it is substituted with at least one size-limited substituent group. In embodiments, when L⁴ is substituted, it is substituted with at least one lower substituent group.

In embodiments, R^{L4} is independently hydrogen, COR^{L4A}, CO₂H, CO₂R^{L4A}, SOR^{L4A}, SO₂R^{L4A}, CONH₂, CONR^{L4A}R^{L4B}, SO₂NH₂, SO₂NR^{L4A}R^{L4B}, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted R^{L4} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{L4} is substituted with a plurality of groups selected from substituent groups, size-limited substituent groups, and lower substituent groups; each substituent group, size-limited substituent group, and/or lower substituent group may optionally be different. In embodiments, when R^{L4} is substituted, it is substituted with at least one substituent group. In embodiments, when R^{L4} is substituted, it is substituted with at least one size-limited substituent group. In embodiments, when R^{L4} is substituted, it is substituted with at least one lower substituent group.

In embodiments, R^{L4A} is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted R^{L4A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{L4A} is substituted with a plurality of groups selected from substituent groups, size-limited substituent groups, and lower substituent groups; each substituent group, size-limited substituent group, and/or lower substituent group may optionally be different. In embodiments, when R^{L4A} is substituted, it is substituted with at least one substituent group. In embodiments, when R^{L4A} is substituted, it is substituted with at least one size-limited substituent group. In embodiments, when R^{L4A} is substituted, it is substituted with at least one lower substituent group.

In embodiments, R^{L4B} is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted R^{L4B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{L4B} is substituted with a plurality of groups selected from substituent groups, size-limited substituent groups, and lower substituent groups; each substituent group, size-limited substituent group, and/or lower substituent group may optionally be different. In embodiments, when R^{L4B} is substituted, it is substituted with at least one substituent group. In embodiments, when R^{L4B} is substituted, it is substituted with at least one size-limited substituent group. In embodiments, when R^{L4B} is substituted, it is substituted with at least one lower substituent group.

In embodiments, a substituted ring formed when R^{L4A} and R^{L4B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{L4A} and R^{L4B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{L4A} and R^{L4B} substituentS bonded to the same nitrogen atom are joined is substituted, it is substituted with at least one substituent group. In embodiments, when the substituted ring formed when R^{L4A} and R^{L4B} substituents bonded to the same nitrogen atom are joined is substituted, it is substituted with at least one size-limited substituent group. In embodiments, when the substituted ring formed when R^{L4A} and R^{L4B} substituents bonded to the same nitrogen atom are joined is substituted, it is substituted with at least one lower substituent group.

In embodiments, R^{L1} is independently hydrogen, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted R^{L1} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{L1} is substituted with a plurality of groups selected from substituent groups, size-limited substituent groups, and lower substituent groups; each substituent group, size-limited substituent group, and/or lower substituent group may optionally be different. In embodiments, when R^{L1} is substituted, it is substituted with at least one substituent group. In embodiments, when R^{L1} is substituted, it is substituted with at least one size-limited substituent group. In embodiments, when R^{L1} is substituted, it is substituted with at least one lower substituent group.

In embodiments, R^{L2} is independently hydrogen, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted R^{L2} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{L2} is substituted with a plurality of groups selected from substituent groups, size-limited substituent groups, and lower substituent groups; each substituent group, size-limited substituent group, and/or lower substituent group may optionally be different. In embodiments, when R^{L2} is substituted, it is substituted with at least one substituent group. In embodiments, when R^{L2} is substituted, it is substituted with at least one size-limited substituent group. In embodiments, when R^{L2} is substituted, it is substituted with at least one lower substituent group.

In embodiments, R⁵ is independently hydrogen, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted R⁵ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁵ is substituted with a plurality of groups selected from substituent groups, size-limited substituent groups, and lower substituent groups; each substituent group, size-limited substituent group, and/or lower substituent group may optionally be different. In embodiments, when R⁵ is substituted, it is substituted with at least one substituent group. In embodiments, when R⁵ is substituted, it is substituted with at least one size-limited substituent group. In embodiments, when R⁵ is substituted, it is substituted with at least one lower substituent group.

In embodiments, R⁶ is independently hydrogen, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted R⁶ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁶ is substituted with a plurality of groups selected from substituent groups, size-limited substituent groups, and lower substituent groups; each substituent group, size-limited substituent group, and/or lower substituent group may optionally be different. In embodiments, when R⁶ is substituted, it is substituted with at least one substituent group. In embodiments, when R⁶ is substituted, it is substituted with at least one size-limited substituent group. In embodiments, when R⁶ is substituted, it is substituted with at least one lower substituent group.

In embodiments, R⁷ is independently hydrogen, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted R⁷ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁷ is substituted with a plurality of groups selected from substituent groups, size-limited substituent groups, and lower substituent groups; each substituent group, size-limited substituent group, and/or lower substituent group may optionally be different. In embodiments, when R⁷ is substituted, it is substituted with at least one substituent group. In embodiments, when R⁷ is substituted, it is substituted with at least one size-limited substituent group. In embodiments, when R⁷ is substituted, it is substituted with at least one lower substituent group.

In embodiments, R⁸ is independently hydrogen, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted R⁸ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁸ is substituted with a plurality of groups selected from substituent groups, size-limited substituent groups, and lower substituent groups; each substituent group, size-limited substituent group, and/or lower substituent group may optionally be different. In embodiments, when R⁸ is substituted, it is substituted with at least one substituent group. In embodiments, when R⁸ is substituted, it is substituted with at least one size-limited substituent group. In embodiments, when R⁸ is substituted, it is substituted with at least one lower substituent group.

In embodiments, R⁹ is independently hydrogen, halogen, —CX⁹₃, —CHX⁹₂, —CH₂X⁹, —OCX⁹₃, —OCH₂X⁹, —OCHX⁹₂, —CN, —SOₙ₉R^{9D}, —SOᵥ₉NR^{9A}R^{9B}, —NR^{9C}R^{9A}R^{9B}, —ONR^{9A}R^{9B}, —NHC(O)NR^{9C}NR^{9A}R^{9B}, —NHC(O)NR^{9A}R^{9B}, —N(O)ₘ₉, —NR^{9A}R^{9B}, —C(O)R^{9C}, —OC(O)R^{9C}, —C(O)—OR^{9C}, —OC(O)—OR^{9C}, —C(O)NR^{9A}R^{9B}, —OC(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, —SF₅, —N₃, O—P(O)(OR^{9A})₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted R⁹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁹ is substituted with a plurality of groups selected from substituent groups, size-limited substituent groups, and lower substituent groups; each substituent group, size-limited substituent group, and/or lower substituent group may optionally be different. In embodiments, when R⁹ is substituted, it is substituted with at least one substituent group. In embodiments, when R⁹ is substituted, it is substituted with at least one size-limited substituent group. In embodiments, when R⁹ is substituted, it is substituted with at least one lower substituent group.

In embodiments, R^{9A} is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted R^{9A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{9A} is substituted with a plurality of groups selected from substituent groups, size-limited substituent groups, and lower substituent groups; each substituent group, size-limited substituent group, and/or lower substituent group may optionally be different. In embodiments, when R^{9A} is substituted, it is substituted with at least one substituent group. In embodiments, when R^{9A} is substituted, it is substituted with at least one size-limited substituent group. In embodiments, when R^{9A} is substituted, it is substituted with at least one lower substituent group.

In embodiments, R^{9B} is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted R^{9B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{9B} is substituted with a plurality of groups selected from substituent groups, size-limited substituent groups, and lower substituent groups; each substituent group, size-limited substituent group, and/or lower substituent group may optionally be different. In embodiments, when R^{9B} is substituted, it is substituted with at least one substituent group. In embodiments, when R^{9B} is substituted, it is substituted with at least one size-limited substituent group. In embodiments, when R^{9B} is substituted, it is substituted with at least one lower substituent group.

In embodiments, a substituted ring formed when R^{9A} and R^{9B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{9A} and R^{9B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{9A} and R^{9B} substituents bonded to the same nitrogen atom are joined is substituted, it is substituted with at least one substituent group. In embodiments, when the substituted ring formed when R^{9A} and R^{9B} substituents bonded to the same nitrogen atom are joined is substituted, it is substituted with at least one size-limited substituent group. In embodiments, when the substituted ring formed when R^{9A} and R^{9B} substituents bonded to the same nitrogen atom are joined is substituted, it is substituted with at least one lower substituent group.

In embodiments, R^{9C} is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted R^{9C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{9C} is substituted with a plurality of groups selected from substituent groups, size-limited substituent groups, and lower substituent groups; each substituent group, size-limited substituent group, and/or lower substituent group may optionally be different. In embodiments, when R^{9C} is substituted, it is substituted with at least one substituent group. In embodiments, when R^{9C} is substituted, it is substituted with at least one size-limited substituent group. In embodiments, when R_{9C} is substituted, it is substituted with at least one lower substituent group.

In embodiments, R^{9D} is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted R^{9D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{9D} is substituted with a plurality of groups selected from substituent groups, size-limited substituent groups, and lower substituent groups; each substituent group, size-limited substituent group, and/or lower substituent group may optionally be different. In embodiments, when R^{9D} is substituted, it is substituted with at least one substituent group. In embodiments, when R^{9D} is substituted, it is substituted with at least one size-limited substituent group. In embodiments, when R^{9D} is substituted, it is substituted with at least one lower substituent group.

In embodiments, L is independently halo-substituted (C₁-C₁₀ saturated alkylene), R^{L}-substituted or unsubstituted C₁-C₁₀ alkylene (e.g., C₁-C₈, C₁-C₆, C₁-C₄, or C₁-C₂), R^{L}-substituted or unsubstituted C₂-C₁₀ alkenylene (e.g., C₂-C₈, C₂-C₆, C₂-C₄, or C₂), R^{L}-substituted or unsubstituted C₂-C₁₀ alkynylene (e.g., C₂-C₈, C₂-C₆, C₂-C₄, or C₂), R^{L}-substituted or unsubstituted C₂-C₆ saturated alkylene-O—C₂-C₆ saturated alkylene, R^{L}-substituted or unsubstituted C₂ saturated alkylene-O—C₂ saturated alkylene, R^{L}-substituted or unsubstituted C₂ saturated alkylene-O—C₂ saturated alkylene-O—C₂ saturated alkylene, R^{L}-substituted or unsubstituted C₂-C₆ saturated alkylene-NR^{L4}—C₂-C₆ saturated alkylene, R^{L}-substituted or unsubstituted C₃-C₆ cycloalkylene, R^{L}-substituted or unsubstituted phenylene, R^{L}-substituted or unsubstituted 4-6 membered heterocycloalkylene, R^{L}-substituted or unsubstituted 5-6 membered heteroarylene, R^{L}-substituted or unsubstituted C₁-C₄ saturated alkylene-(C₃-C₆ cycloalkylene)-C₁-C₄ saturated alkylene, R^{L}-substituted or unsubstituted C₁-C₄ saturated alkylene-phenylene-C₁-C₄ saturated alkylene, R^{L}-substituted or unsubstituted C₁-C₄ saturated alkylene-(4-6 membered heterocycloalkylene)-C₁-C₄ saturated alkylene, or R^{L}-substituted or unsubstituted C₁-C₄ saturated alkylene-(5-6 membered heteroarylene)-C₁-C₄ saturated alkylene.

In embodiments, a substituted L (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L is substituted with a plurality of groups selected from substituent groups, size-limited substituent groups, and lower substituent groups; each substituent group, size-limited substituent group, and/or lower substituent group may optionally be different. In embodiments, when L is substituted, it is substituted with at least one substituent group. In embodiments, when L is substituted, it is substituted with at least one size-limited substituent group. In embodiments, when L is substituted, it is substituted with at least one lower substituent group.

In embodiments, R^{90A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy.

In embodiments, a substituted R^{90A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{90A} is substituted with a plurality of groups selected from substituent groups, size-limited substituent groups, and lower substituent groups; each substituent group, size-limited substituent group, and/or lower substituent group may optionally be different. In embodiments, when R^{90A} is substituted, it is substituted with at least one substituent group. In embodiments, when R^{90A} is substituted, it is substituted with at least one size-limited substituent group. In embodiments, when R^{90A} is substituted, it is substituted with at least one lower substituent group.

In embodiments, R^{90B} is independently hydrogen, unsubstituted C₁-C₁₀ saturated alkyl (e.g., C₁-C₈, C₁-C₆, C₁-C₄, or C₁-C₂), unsubstituted C₂-C₁₀ alkenyl (e.g., C₂-C₈, C₂-C₆, C₂-C₄, or C₂), unsubstituted C₂-C₁₀ alkynyl (e.g., C₂-C₈, C₂-C₆, C₂-C₄, or C₂), C₁-C₁₀ hydroxy-substituted saturated alkyl (e.g., C₁-C₈, C₁-C₆, C₁-C₄, or C₁-C₂), R⁹¹-substituted or unsubstituted phenyl, R⁹¹-substituted or unsubstituted heteroaryl (e.g., 5 to 10 membered, 5 to 9 membered, or 5 to 6 membered), R⁹¹-substituted or unsubstituted heterocycloalkyl, or R⁹¹-substituted or unsubstituted heteroaryl; wherein said R⁹¹-substituted or unsubstituted phenyl, R⁹¹-substituted or unsubstituted heteroaryl, R⁹¹-substituted or unsubstituted heterocycloalkyl, and R⁹¹-substituted or unsubstituted heteroaryl are optionally substituted with one or more R⁹¹ substituents.

In embodiments, a substituted R^{90B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{90B} is substituted with a plurality of groups selected from substituent groups, size-limited substituent groups, and lower substituent groups; each substituent group, size-limited substituent group, and/or lower substituent group may optionally be different. In embodiments, when R^{90B} is substituted, it is substituted with at least one substituent group. In embodiments, when R^{90B} is substituted, it is substituted with at least one size-limited substituent group. In embodiments, when R^{90B} is substituted, it is substituted with at least one lower substituent group.

In embodiments, R^{90J} is independently hydrogen, unsubstituted C₁-C₁₀ saturated alkyl (e.g., C₁-C₈, C₁-C₆, C₁-C₄, or C₁-C₂), unsubstituted C₂-C₁₀ alkenyl (e.g., C₂-C₈, C₂-C₆, C₂-C₄, or C₂), unsubstituted C₂-C₁₀ alkynyl (e.g., C₂-C₈, C₂-C₆, C₂-C₄, or C₂), C₁-C₁₀ hydroxy-substituted saturated alkyl (e.g., C₁-C₈, C₁-C₆, C₁-C₄, or C₁-C₂), substituted or unsubstituted aryl (e.g., C₆-C₁₀ or phenyl), substituted or unsubstituted heteroaryl (e.g., 5 to 10 membered, 5 to 9 membered, or 5 to 6 membered), or substituted or unsubstituted heterocycloalkyl (e.g., 3 to 8 membered, 3 to 6 membered, 4 to 6 membered, 4 to 5 membered, or 5 to 6 membered), wherein said substituted aryl, substituted heteroaryl, and substituted heterocyclic groups are independently substituted with one or more halogen, CN, CF₃, or unsubstituted C₁-C₄ saturated alkyl.

In embodiments, a substituted R^{90J} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{90J} is substituted with a plurality of groups selected from substituent groups, size-limited substituent groups, and lower substituent groups; each substituent group, size-limited substituent group, and/or lower substituent group may optionally be different. In embodiments, when R^{90J} is substituted, it is substituted with at least one substituent group. In embodiments, when R^{90J} is substituted, it is substituted with at least one size-limited substituent group. In embodiments, when R^{90J} is substituted, it is substituted with at least one lower substituent group.

In embodiments, the compound is one of

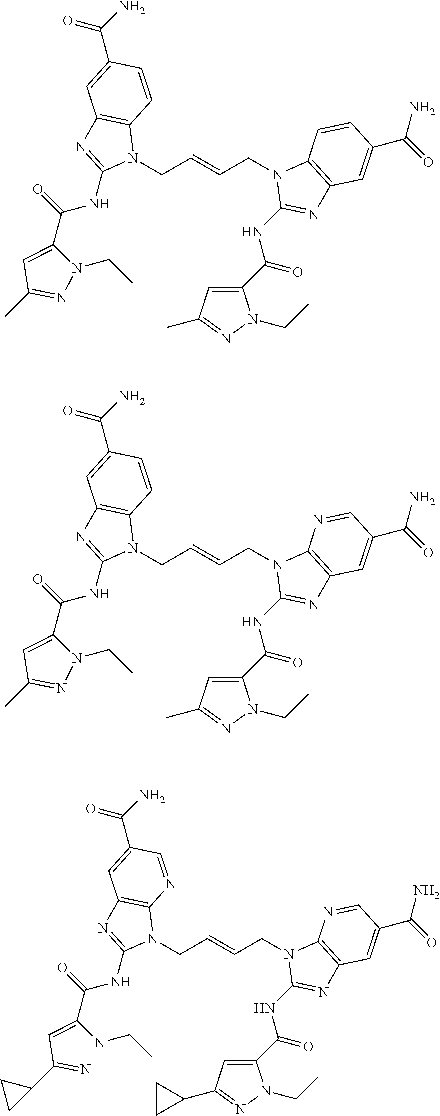 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)ccc21 ; CCn1nc(C)cc1C(=O)Nc1nc2cc(C(N)=O)ccc2n1C/C=C/Cn1c(NC(=O)c2cc(C)nn2CC)nc2cc(C(N)=O)cnc21 ; CCN/C(=C\C(=N)C1CC1)C(=O)Nc1nc2cc(C(N)=O)cnc2n1C/C=C/Cn1c(NC(=O)c2cc(C3CC3)nn2CC)nc2cc(C(N)=O)cnc21]

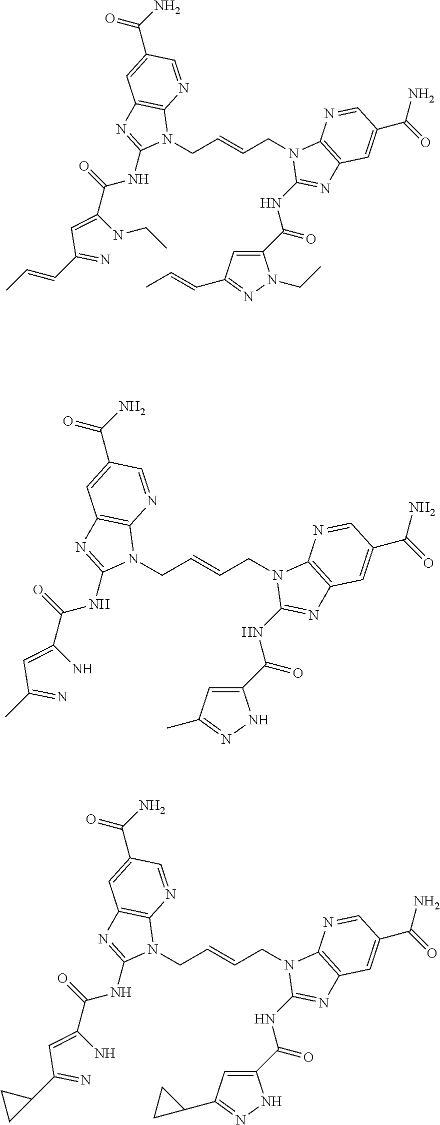 [SMILES: C/C=C/C(=N)/C=C(\NCC)C(=O)Nc1nc2cc(C(N)=O)cnc2n1C/C=C/Cn1c(NC(=O)c2cc(/C=C/C)nn2CC)nc2cc(C(N)=O)cnc21 ; CC(=N)/C=C(\N)C(=O)Nc1nc2cc(C(N)=O)cnc2n1C/C=C/Cn1c(NC(=O)c2cc(C)n[nH]2)nc2cc(C(N)=O)cnc21 ; N=C(/C=C(\N)C(=O)Nc1nc2cc(C(N)=O)cnc2n1C/C=C/Cn1c(NC(=O)c2cc(C3CC3)n[nH]2)nc2cc(C(N)=O)cnc21)C1CC1]

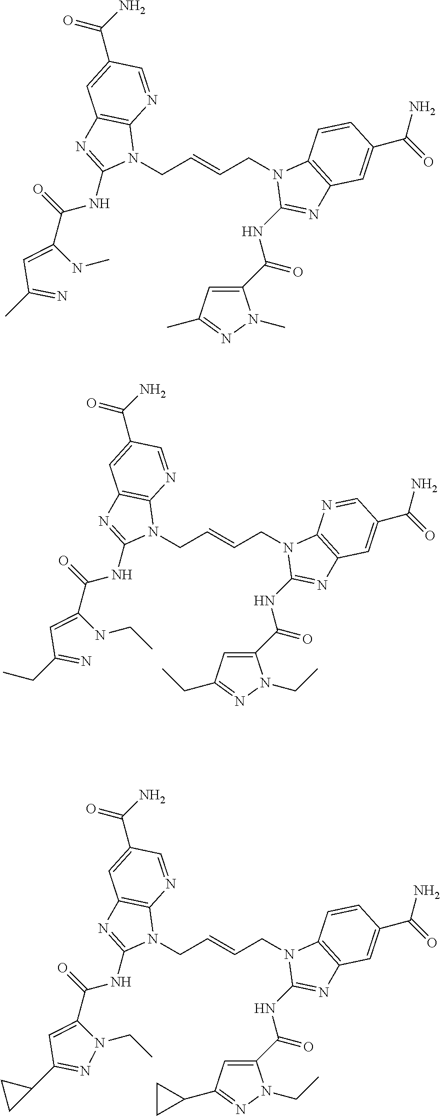 [SMILES: CN/C(=C\C(C)=N)C(=O)Nc1nc2cc(C(N)=O)cnc2n1C/C=C/Cn1c(NC(=O)c2cc(C)nn2C)nc2cc(C(N)=O)ccc21 ; CCN/C(=C\C(=N)CC)C(=O)Nc1nc2cc(C(N)=O)cnc2n1C/C=C/Cn1c(NC(=O)c2cc(CC)nn2CC)nc2cc(C(N)=O)cnc21 ; CCn1nc(C2CC2)cc1C(=O)Nc1nc2cc(C(N)=O)ccc2n1C/C=C/Cn1c(NC(=O)c2cc(C3CC3)nn2CC)nc2cc(C(N)=O)cnc21]

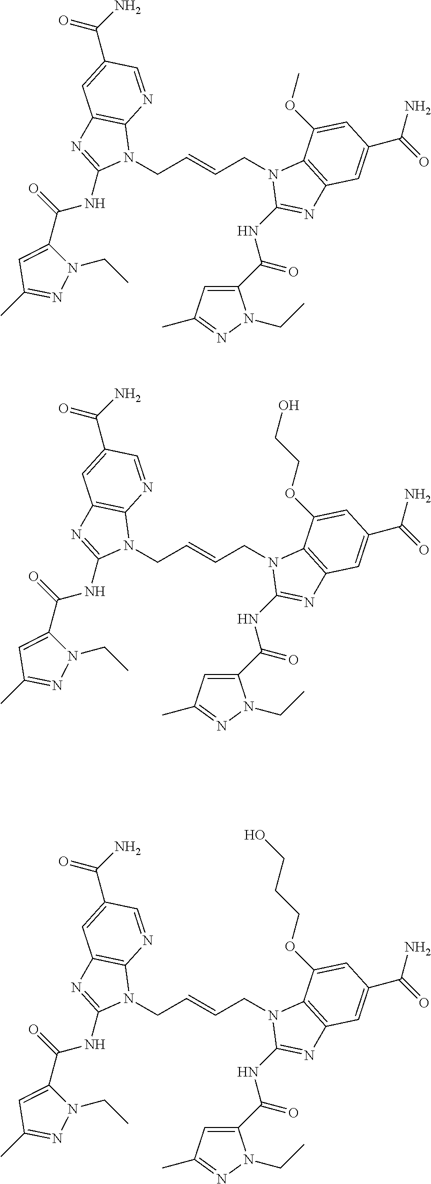 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OC)c21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCO)c21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCO)c21]

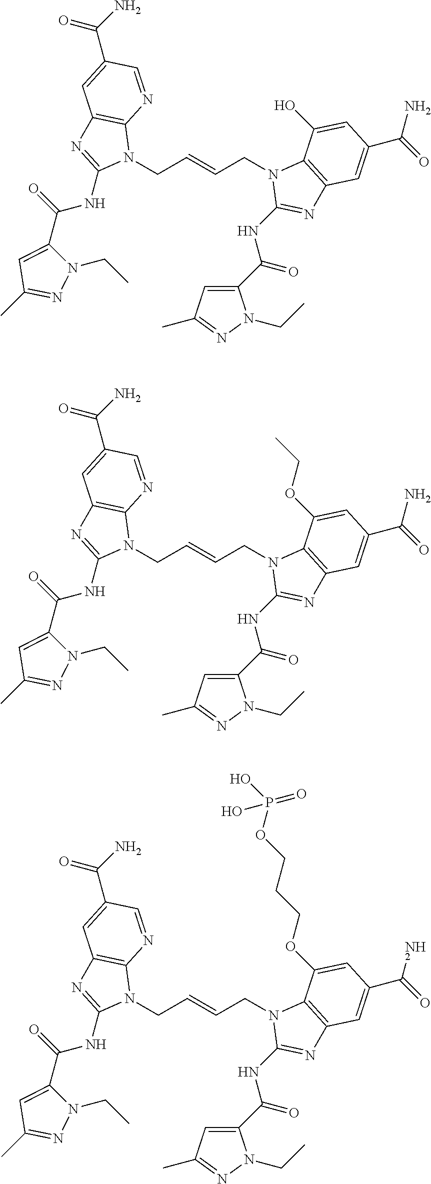 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(O)c21 ; CCOc1cc(C(N)=O)cc2nc(NC(=O)c3cc(C)nn3CC)n(C/C=C/Cn3c(NC(=O)c4cc(C)nn4CC)nc4cc(C(N)=O)cnc43)c12 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCOP(=O)(O)O)c21]

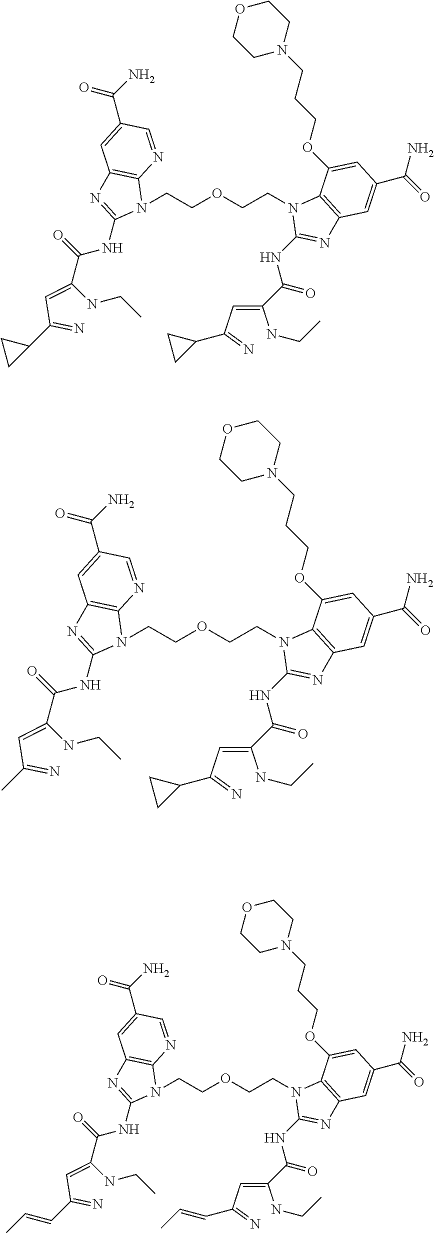 [SMILES: CCN/C(=C\C(=N)C1CC1)C(=O)Nc1nc2cc(C(N)=O)cnc2n1CCOCCn1c(NC(=O)/C(=C/C(=N)C2CC2)NCC)nc2cc(C(N)=O)cc(OCCCN3CCOCC3)c21 ; CCN/C(=C\C(C)=N)C(=O)Nc1nc2cc(C(N)=O)cnc2n1CCOCCn1c(NC(=O)/C(=C/C(=N)C2CC2)NCC)nc2cc(C(N)=O)cc(OCCCN3CCOCC3)c21 ; C/C=C/C(=N)/C=C(\NCC)C(=O)Nc1nc2cc(C(N)=O)cnc2n1CCOCCn1c(NC(=O)/C(=C/C(=N)/C=C/C)NCC)nc2cc(C(N)=O)cc(OCCCN3CCOCC3)c21]

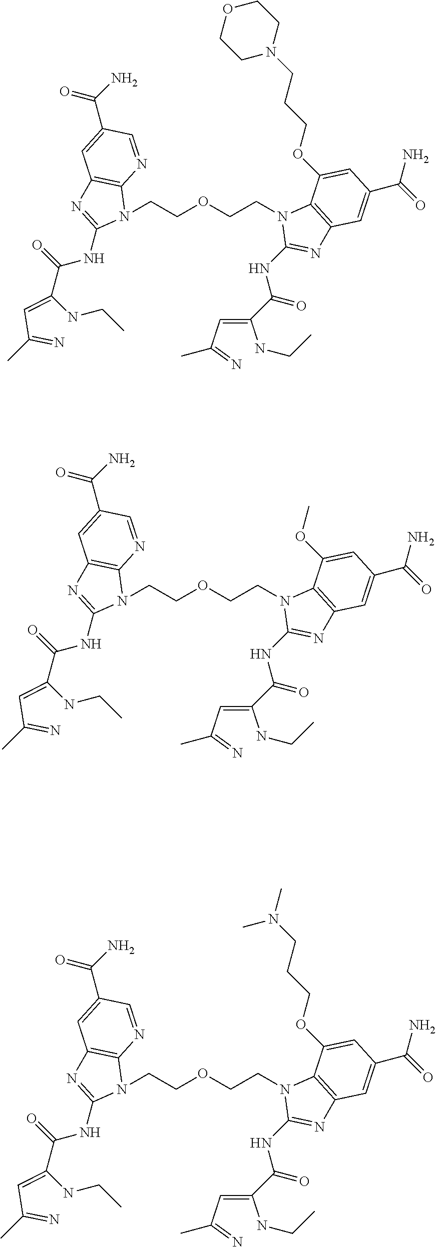 [SMILES: CCN/C(=C\C(C)=N)C(=O)Nc1nc2cc(C(N)=O)cnc2n1CCOCCn1c(NC(=O)/C(=C/C(C)=N)NCC)nc2cc(C(N)=O)cc(OCCCN3CCOCC3)c21 ; CCN/C(=C\C(C)=N)C(=O)Nc1nc2cc(C(N)=O)cnc2n1CCOCCn1c(NC(=O)/C(=C/C(C)=N)NCC)nc2cc(C(N)=O)cc(OC)c21 ; CCN/C(=C\C(C)=N)C(=O)Nc1nc2cc(C(N)=O)cnc2n1CCOCCn1c(NC(=O)/C(=C/C(C)=N)NCC)nc2cc(C(N)=O)cc(OCCCN(C)C)c21]

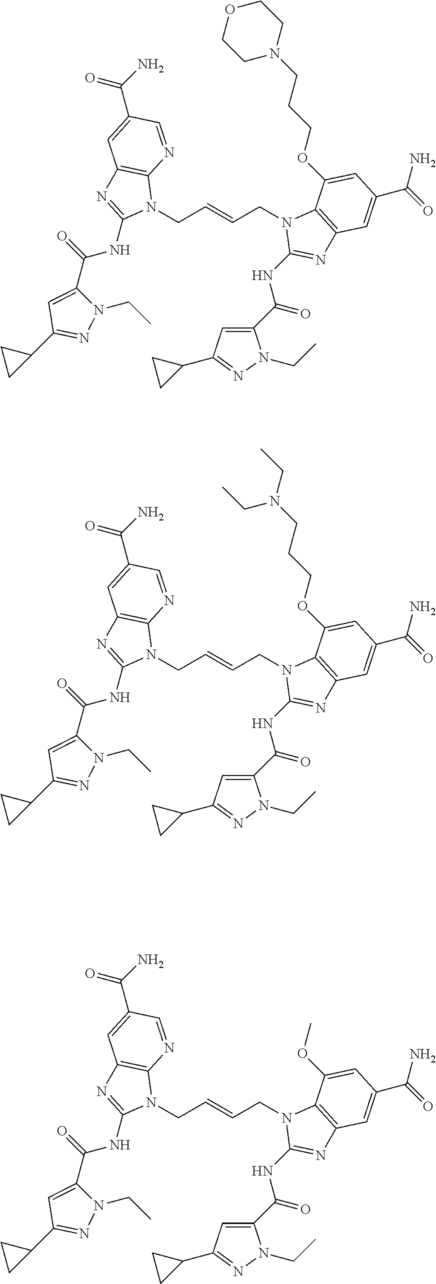 [SMILES: CCn1nc(C2CC2)cc1C(=O)Nc1nc2cc(C(N)=O)cnc2n1C/C=C/Cn1c(NC(=O)c2cc(C3CC3)nn2CC)nc2cc(C(N)=O)cc(OCCCN3CCOCC3)c21 ; CCN(CC)CCCOc1cc(C(N)=O)cc2nc(NC(=O)c3cc(C4CC4)nn3CC)n(C/C=C/Cn3c(NC(=O)c4cc(C5CC5)nn4CC)nc4cc(C(N)=O)cnc43)c12 ; CCn1nc(C2CC2)cc1C(=O)Nc1nc2cc(C(N)=O)cnc2n1C/C=C/Cn1c(NC(=O)c2cc(C3CC3)nn2CC)nc2cc(C(N)=O)cc(OC)c21]

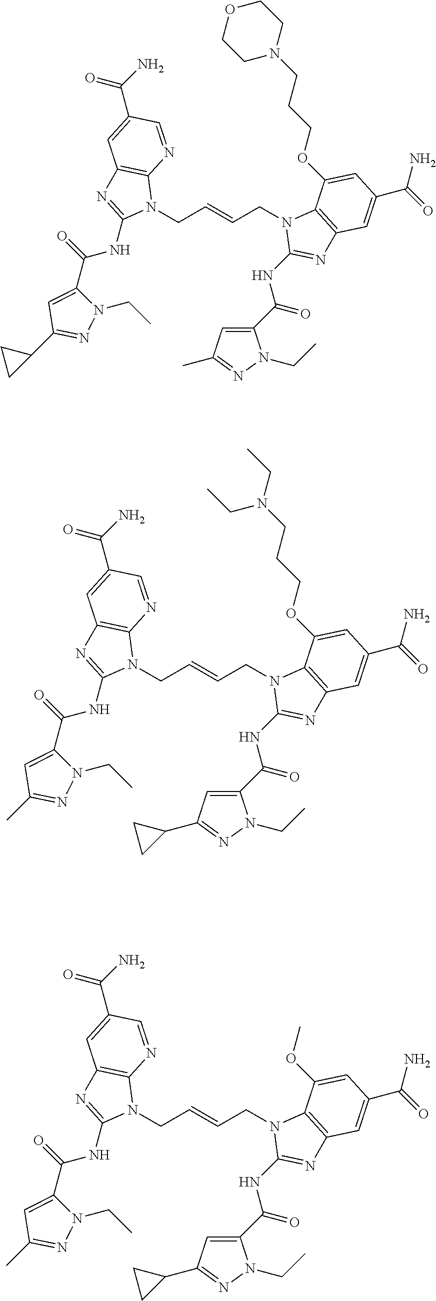 [SMILES: CCn1nc(C2CC2)cc1C(=O)Nc1nc2cc(C(N)=O)cnc2n1C/C=C/Cn1c(NC(=O)c2cc(C)nn2CC)nc2cc(C(N)=O)cc(OCCCN3CCOCC3)c21 ; CCN(CC)CCCOc1cc(C(N)=O)cc2nc(NC(=O)c3cc(C4CC4)nn3CC)n(C/C=C/Cn3c(NC(=O)c4cc(C)nn4CC)nc4cc(C(N)=O)cnc43)c12 ; CCn1nc(C)cc1C(=O)Nc1nc2cc(C(N)=O)cnc2n1C/C=C/Cn1c(NC(=O)c2cc(C3CC3)nn2CC)nc2cc(C(N)=O)cc(OC)c21]

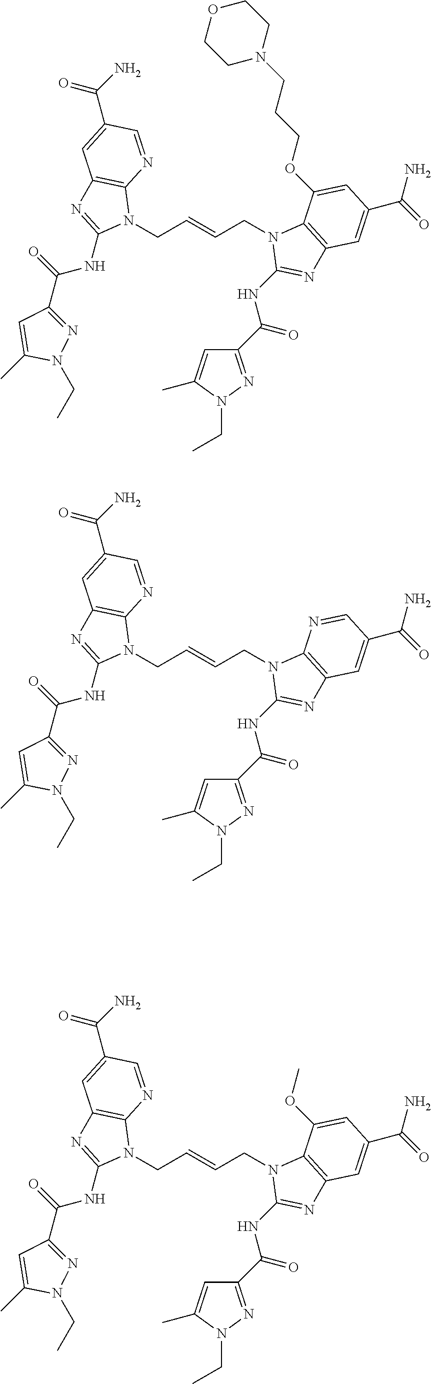 [SMILES: CCn1nc(C(=O)Nc2nc3cc(C(N)=O)cnc3n2C/C=C/Cn2c(NC(=O)c3cc(C)n(CC)n3)nc3cc(C(N)=O)cc(OCCCN4CCOCC4)c32)cc1C ; CCn1nc(C(=O)Nc2nc3cc(C(N)=O)cnc3n2C/C=C/Cn2c(NC(=O)c3cc(C)n(CC)n3)nc3cc(C(N)=O)cnc32)cc1C ; CCn1nc(C(=O)Nc2nc3cc(C(N)=O)cnc3n2C/C=C/Cn2c(NC(=O)c3cc(C)n(CC)n3)nc3cc(C(N)=O)cc(OC)c32)cc1C]

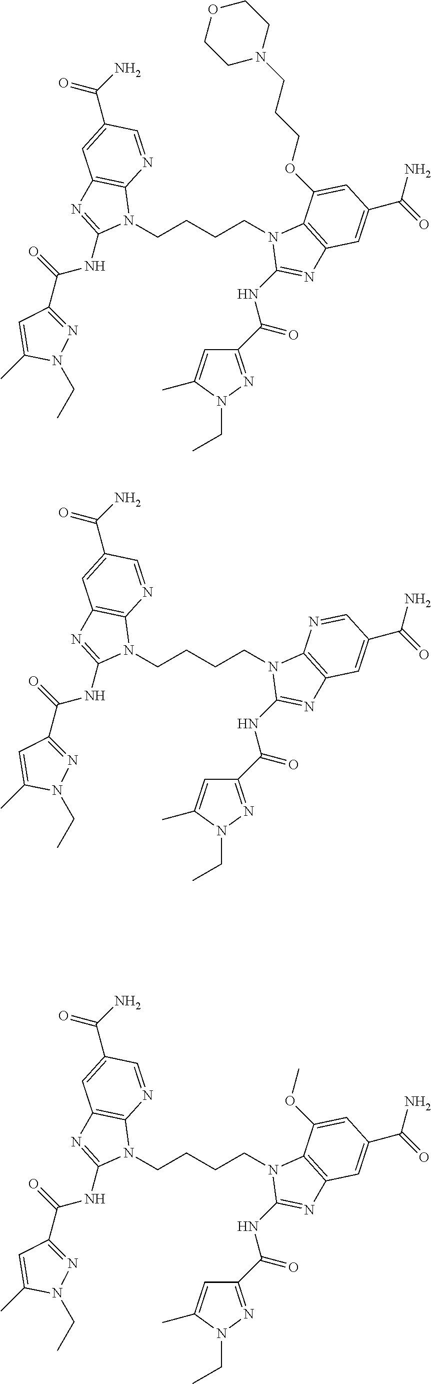 [SMILES: CCn1nc(C(=O)Nc2nc3cc(C(N)=O)cnc3n2CCCCn2c(NC(=O)c3cc(C)n(CC)n3)nc3cc(C(N)=O)cc(OCCCN4CCOCC4)c32)cc1C ; CCn1nc(C(=O)Nc2nc3cc(C(N)=O)cnc3n2CCCCn2c(NC(=O)c3cc(C)n(CC)n3)nc3cc(C(N)=O)cnc32)cc1C ; CCn1nc(C(=O)Nc2nc3cc(C(N)=O)cnc3n2CCCCn2c(NC(=O)c3cc(C)n(CC)n3)nc3cc(C(N)=O)cc(OC)c32)cc1C]

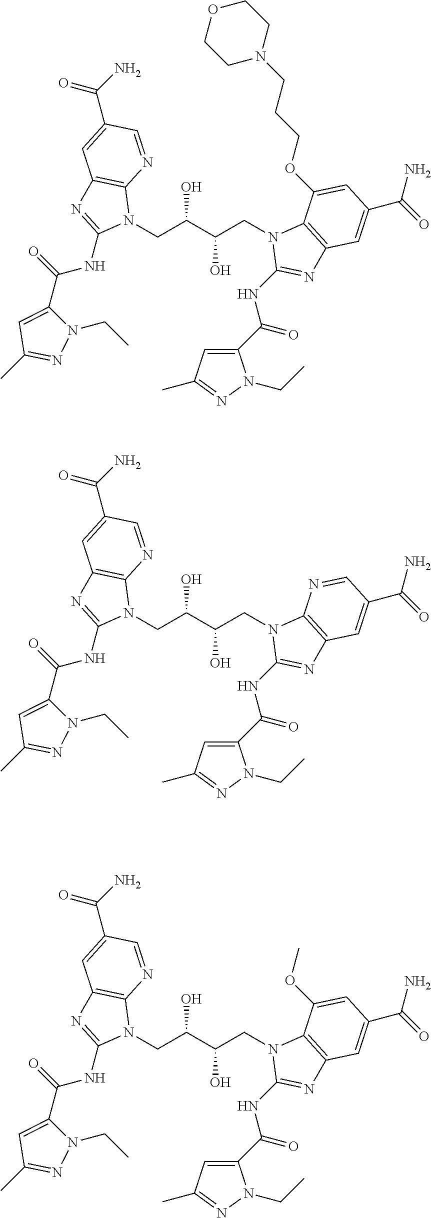 [SMILES: CCn1nc(C)cc1C(=O)Nc1nc2cc(C(N)=O)cnc2n1C[C@H](O)[C@@H](O)Cn1c(NC(=O)c2cc(C)nn2CC)nc2cc(C(N)=O)cc(OCCCN3CCOCC3)c21 ; CCn1nc(C)cc1C(=O)Nc1nc2cc(C(N)=O)cnc2n1C[C@H](O)[C@@H](O)Cn1c(NC(=O)c2cc(C)nn2CC)nc2cc(C(N)=O)cnc21 ; CCn1nc(C)cc1C(=O)Nc1nc2cc(C(N)=O)cnc2n1C[C@H](O)[C@@H](O)Cn1c(NC(=O)c2cc(C)nn2CC)nc2cc(C(N)=O)cc(OC)c21]

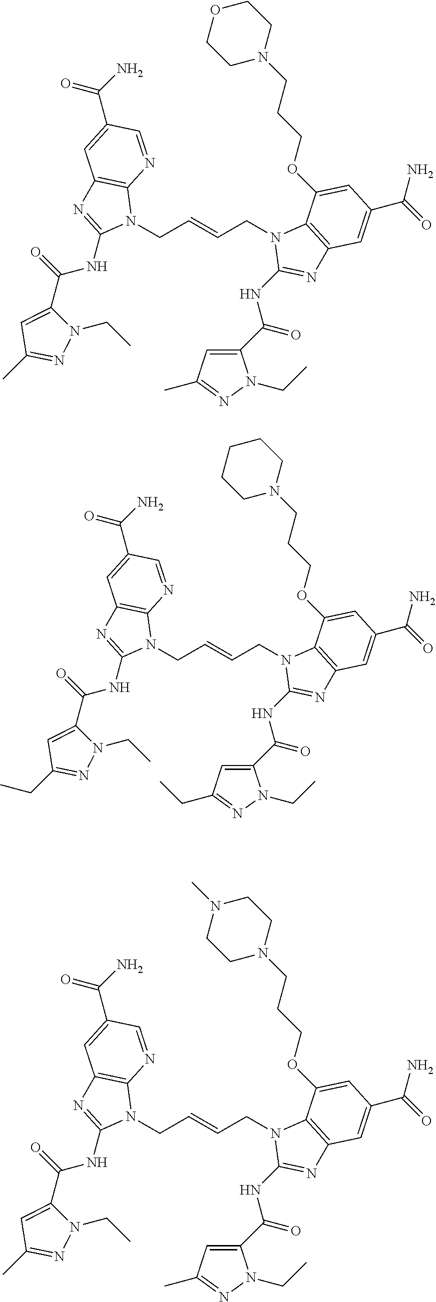

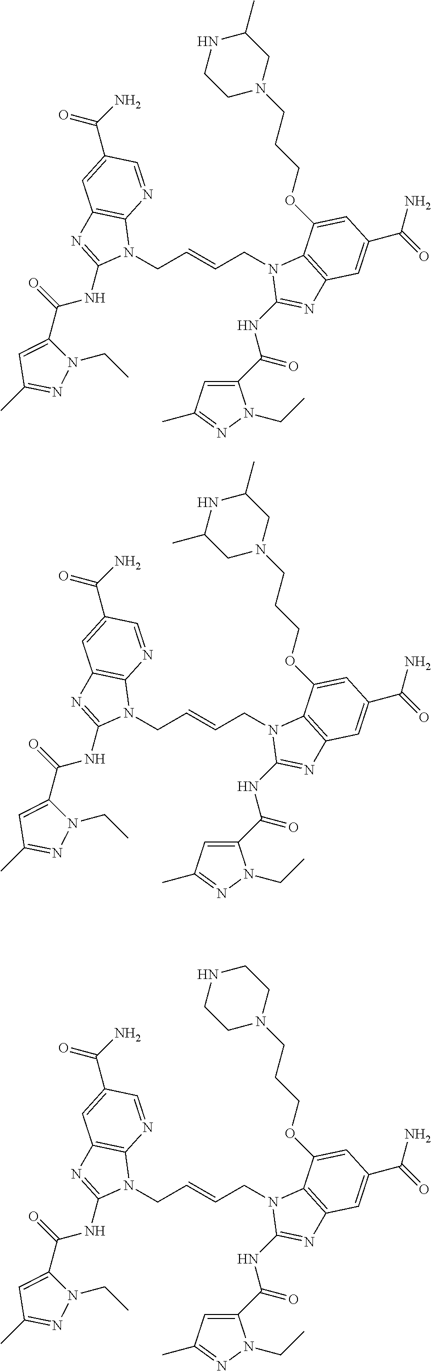

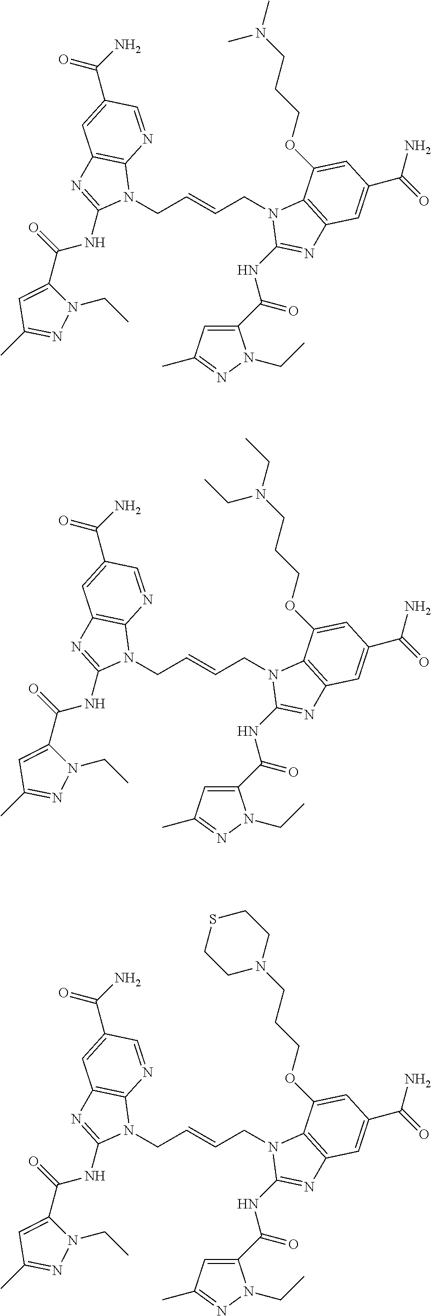 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN(C)C)c21 ; CCN(CC)CCCOc1cc(C(N)=O)cc2nc(NC(=O)c3cc(C)nn3CC)n(C/C=C/Cn3c(NC(=O)c4cc(C)nn4CC)nc4cc(C(N)=O)cnc43)c12 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCSCC3)c21]

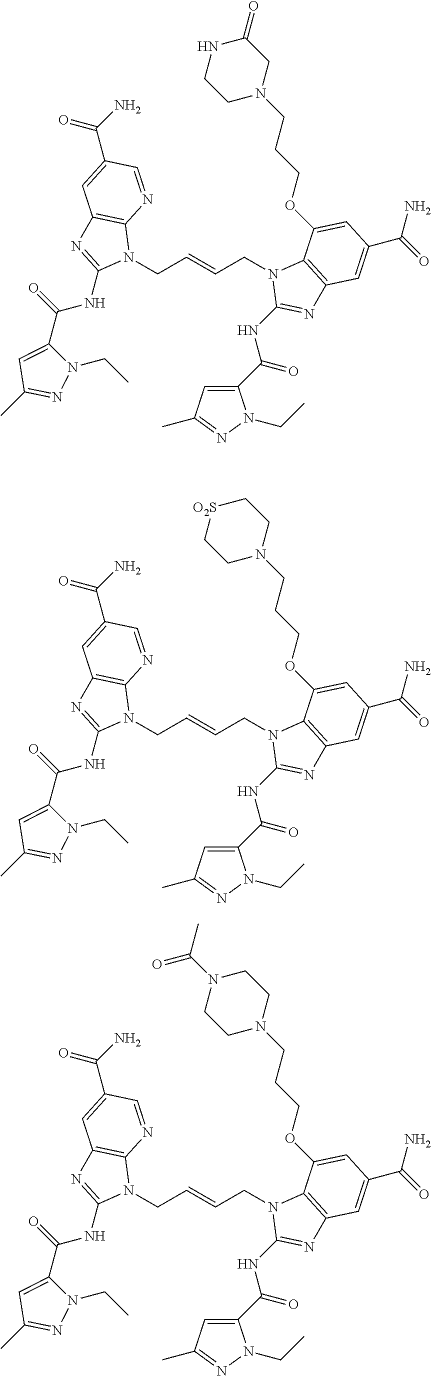

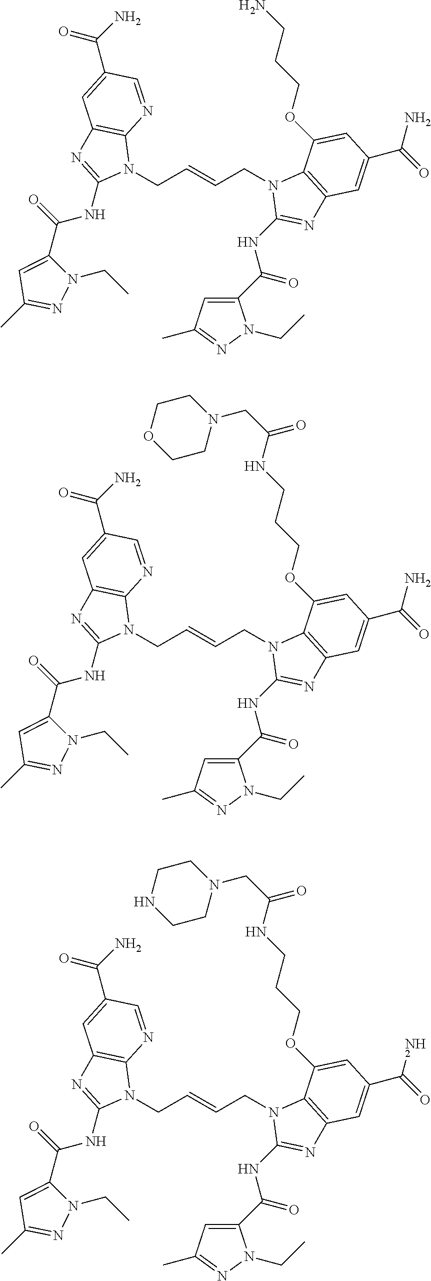 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN)c21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCNC(=O)CN3CCOCC3)c21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCNC(=O)CN3CCNCC3)c21]

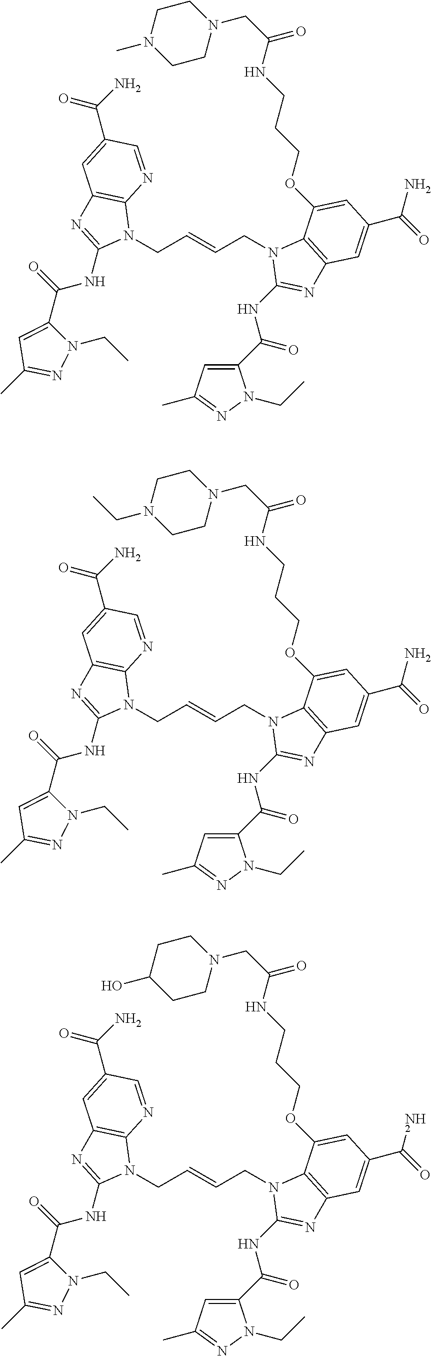 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCNC(=O)CN3CCN(C)CC3)c21 ; CCN1CCN(CC(=O)NCCCOc2cc(C(N)=O)cc3nc(NC(=O)c4cc(C)nn4CC)n(C/C=C/Cn4c(NC(=O)c5cc(C)nn5CC)nc5cc(C(N)=O)cnc54)c23)CC1 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCNC(=O)CN3CCC(O)CC3)c21]

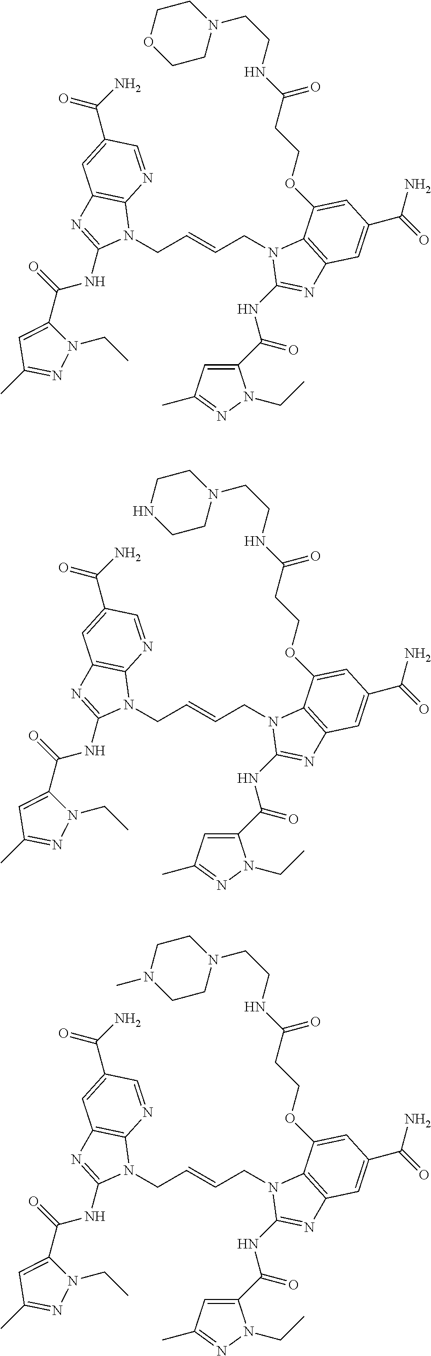 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCC(=O)NCCN3CCOCC3)c21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCC(=O)NCCN3CCNCC3)c21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCC(=O)NCCN3CCN(C)CC3)c21]

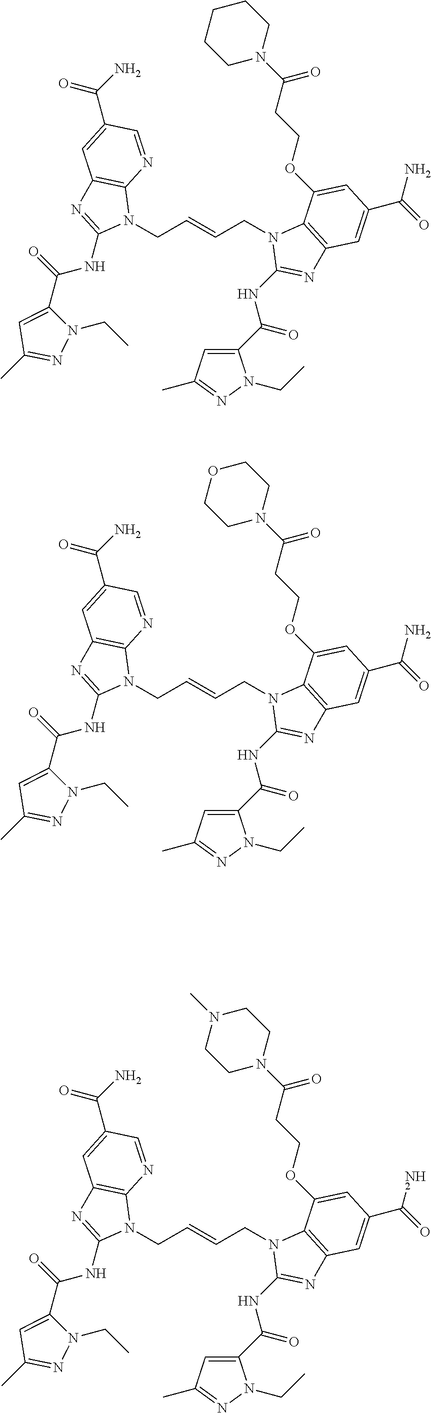 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCC(=O)N3CCCCC3)c21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCC(=O)N3CCOCC3)c21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCC(=O)N3CCN(C)CC3)c21]

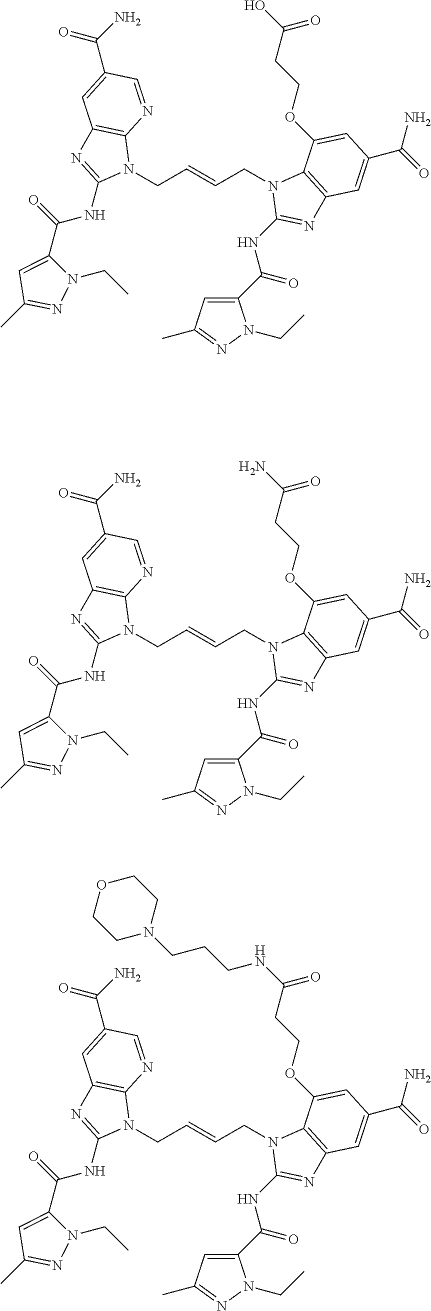 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCC(=O)O)c21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCC(N)=O)c21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCC(=O)NCCCN3CCOCC3)c21]

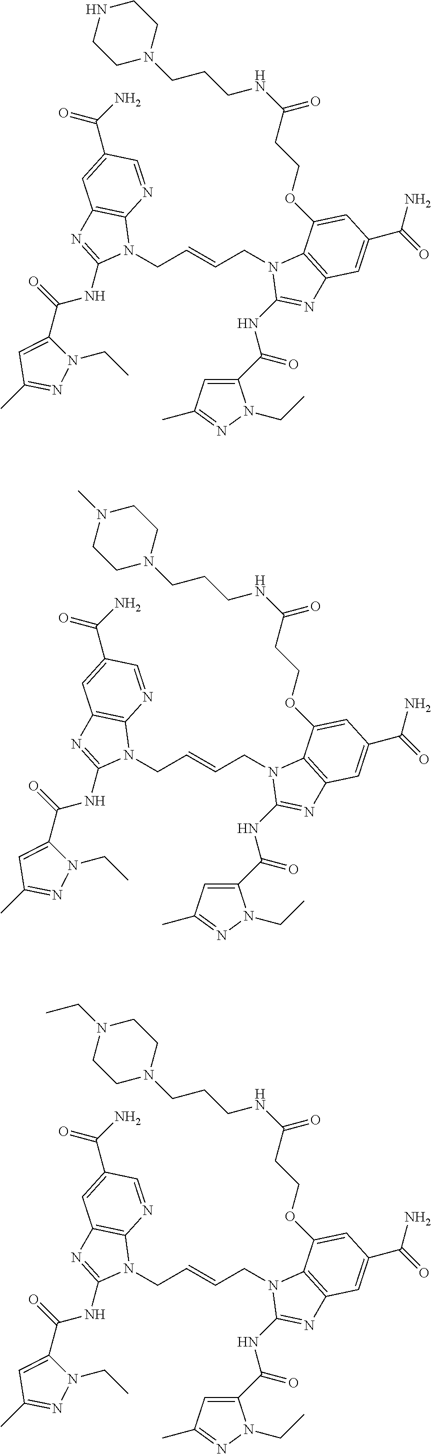 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCC(=O)NCCCN3CCNCC3)c21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCC(=O)NCCCN3CCN(C)CC3)c21 ; CCN1CCN(CCCNC(=O)CCOc2cc(C(N)=O)cc3nc(NC(=O)c4cc(C)nn4CC)n(C/C=C/Cn4c(NC(=O)c5cc(C)nn5CC)nc5cc(C(N)=O)cnc54)c23)CC1]

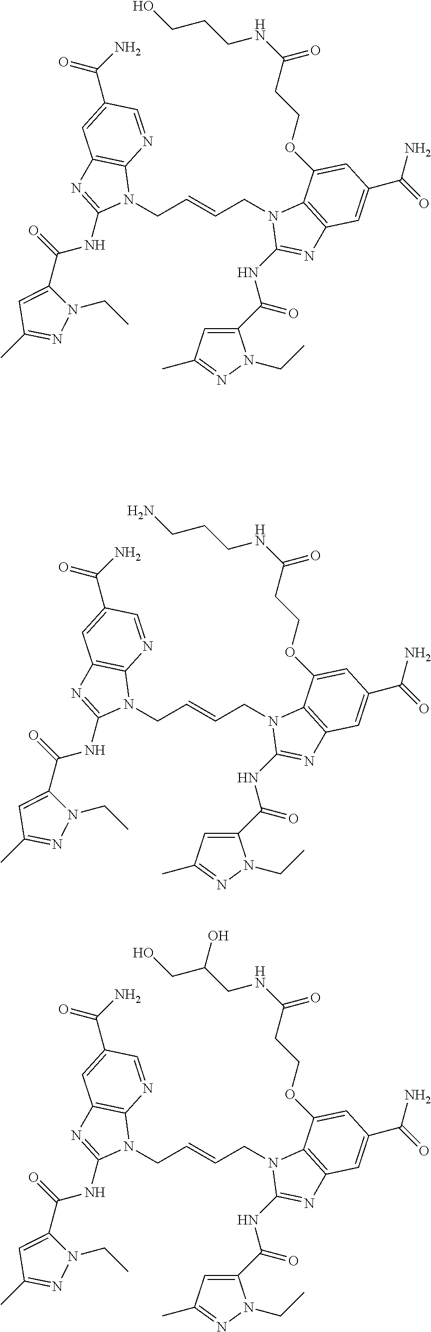 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCC(=O)NCCCO)c21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCC(=O)NCCCN)c21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCC(=O)NCC(O)CO)c21]

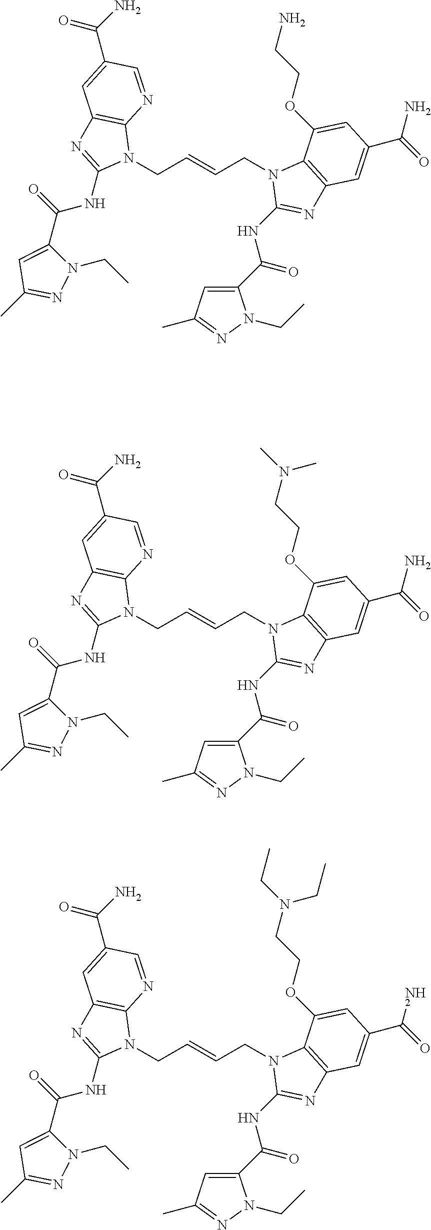 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCN)c21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCN(C)C)c21 ; CCN(CC)CCOc1cc(C(N)=O)cc2nc(NC(=O)c3cc(C)nn3CC)n(C/C=C/Cn3c(NC(=O)c4cc(C)nn4CC)nc4cc(C(N)=O)cnc43)c12]

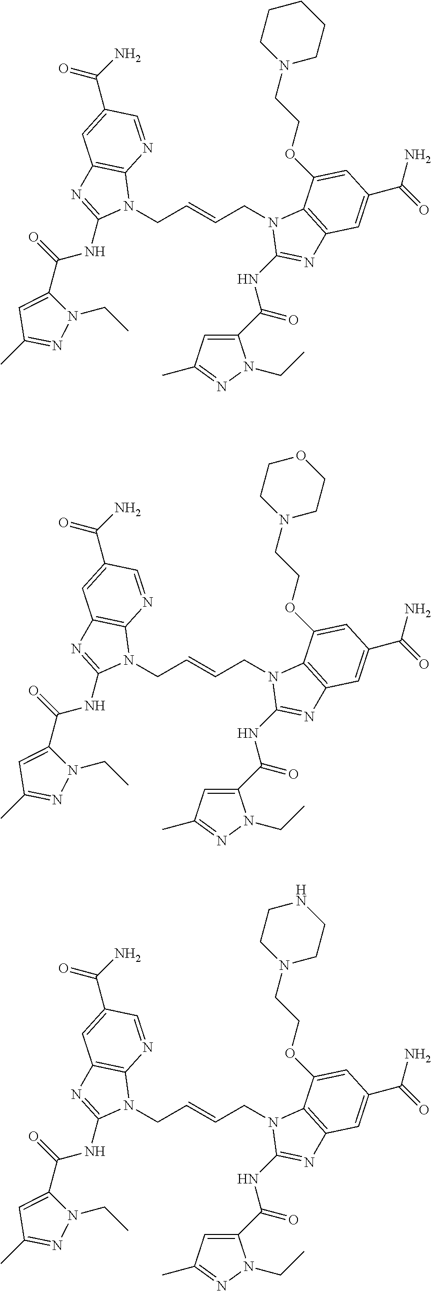 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCN3CCCCC3)c21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCN3CCOCC3)c21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCN3CCNCC3)c21]

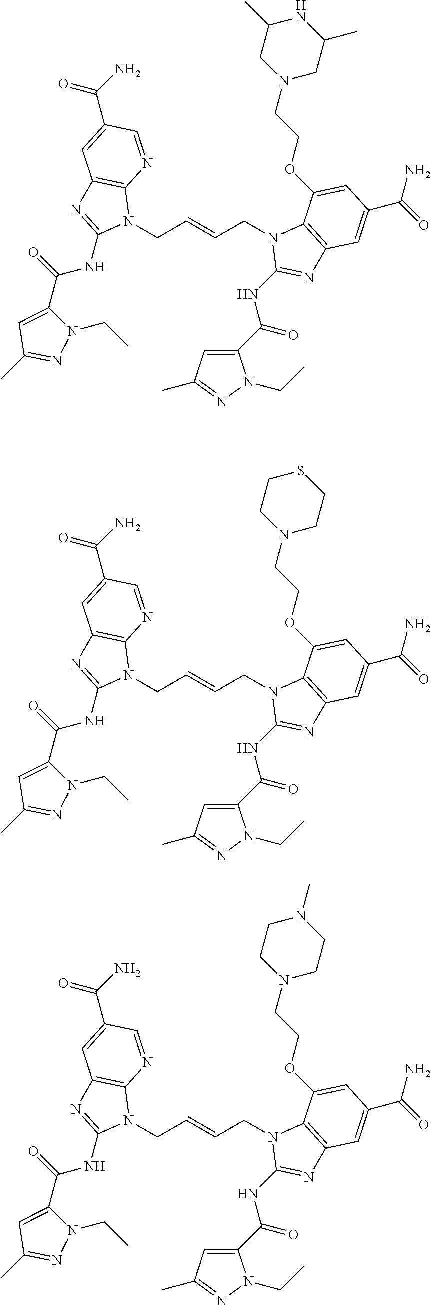 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCN3CC(C)NC(C)C3)c21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCN3CCSCC3)c21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCN3CCN(C)CC3)c21]

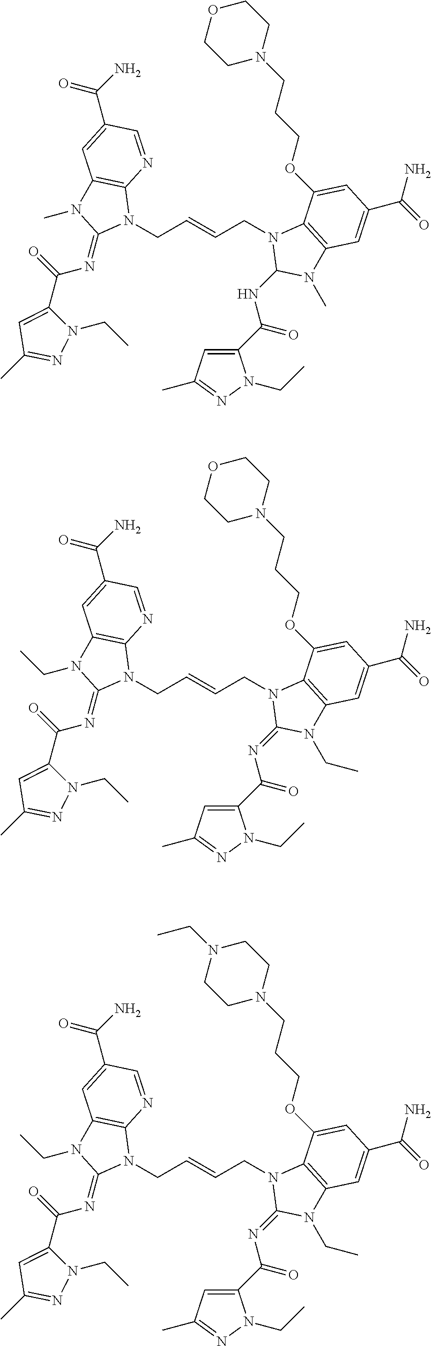 [SMILES: CCn1nc(C)cc1C(=O)/N=c1\n(C)c2cc(C(N)=O)cnc2n1C/C=C/CN1c2c(OCCCN3CCOCC3)cc(C(N)=O)cc2N(C)C1NC(=O)c1cc(C)nn1CC ; CCn1nc(C)cc1C(=O)/N=c1\n(CC)c2cc(C(N)=O)cnc2n1C/C=C/Cn1/c(=N/C(=O)c2cc(C)nn2CC)n(CC)c2cc(C(N)=O)cc(OCCCN3CCOCC3)c21 ; CCN1CCN(CCCOc2cc(C(N)=O)cc3c2n(C/C=C/Cn2/c(=N/C(=O)c4cc(C)nn4CC)n(CC)c4cc(C(N)=O)cnc42)/c(=N/C(=O)c2cc(C)nn2CC)n3CC)CC1]

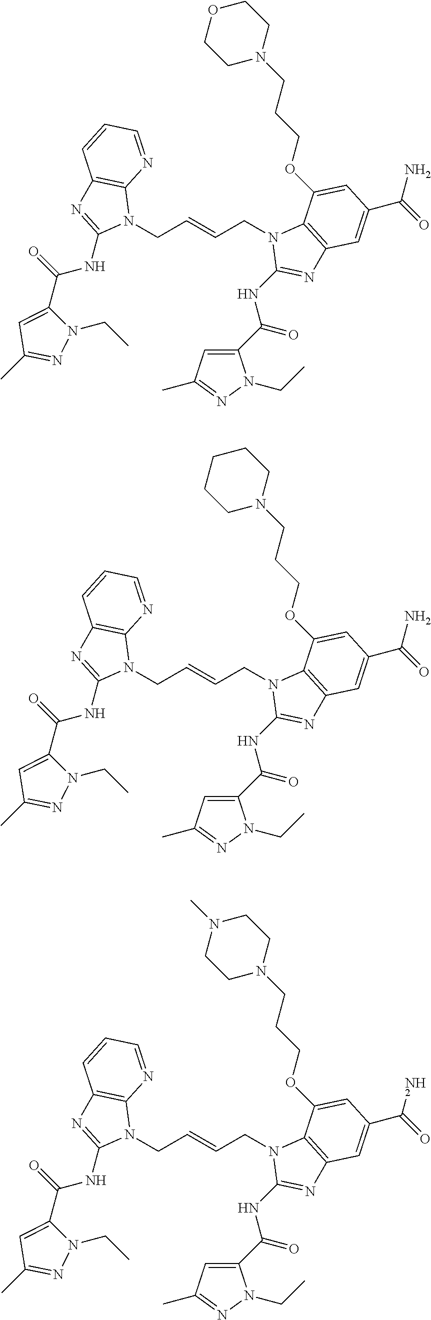 [SMILES: CCn1nc(C)cc1C(=O)Nc1nc2cccnc2n1C/C=C/Cn1c(NC(=O)c2cc(C)nn2CC)nc2cc(C(N)=O)cc(OCCCN3CCOCC3)c21 ; CCn1nc(C)cc1C(=O)Nc1nc2cccnc2n1C/C=C/Cn1c(NC(=O)c2cc(C)nn2CC)nc2cc(C(N)=O)cc(OCCCN3CCCCC3)c21 ; CCn1nc(C)cc1C(=O)Nc1nc2cccnc2n1C/C=C/Cn1c(NC(=O)c2cc(C)nn2CC)nc2cc(C(N)=O)cc(OCCCN3CCN(C)CC3)c21]

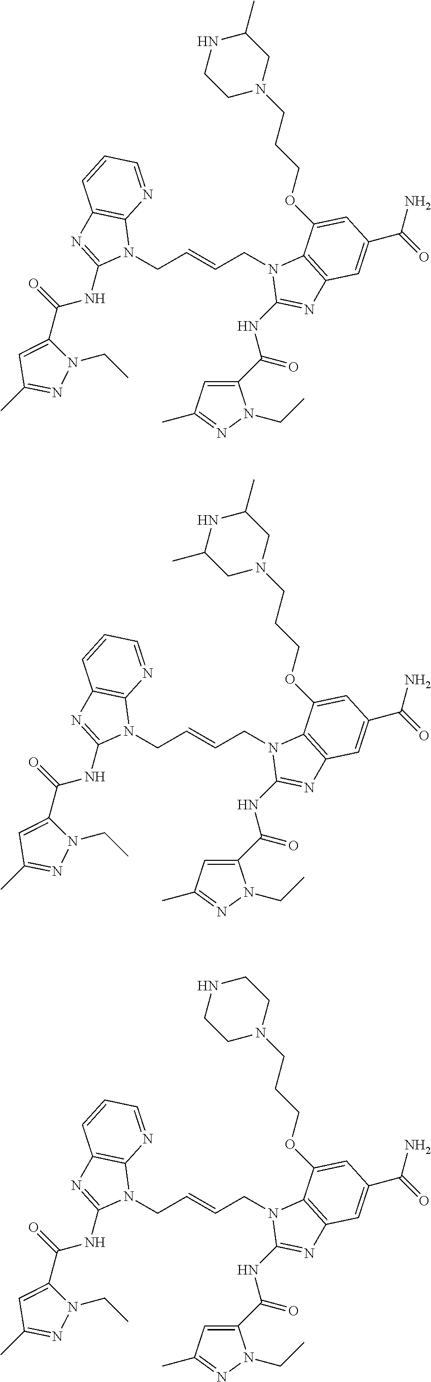 [SMILES: CCn1nc(C)cc1C(=O)Nc1nc2cccnc2n1C/C=C/Cn1c(NC(=O)c2cc(C)nn2CC)nc2cc(C(N)=O)cc(OCCCN3CCNC(C)C3)c21 ; CCn1nc(C)cc1C(=O)Nc1nc2cccnc2n1C/C=C/Cn1c(NC(=O)c2cc(C)nn2CC)nc2cc(C(N)=O)cc(OCCCN3CC(C)NC(C)C3)c21 ; CCn1nc(C)cc1C(=O)Nc1nc2cccnc2n1C/C=C/Cn1c(NC(=O)c2cc(C)nn2CC)nc2cc(C(N)=O)cc(OCCCN3CCNCC3)c21]

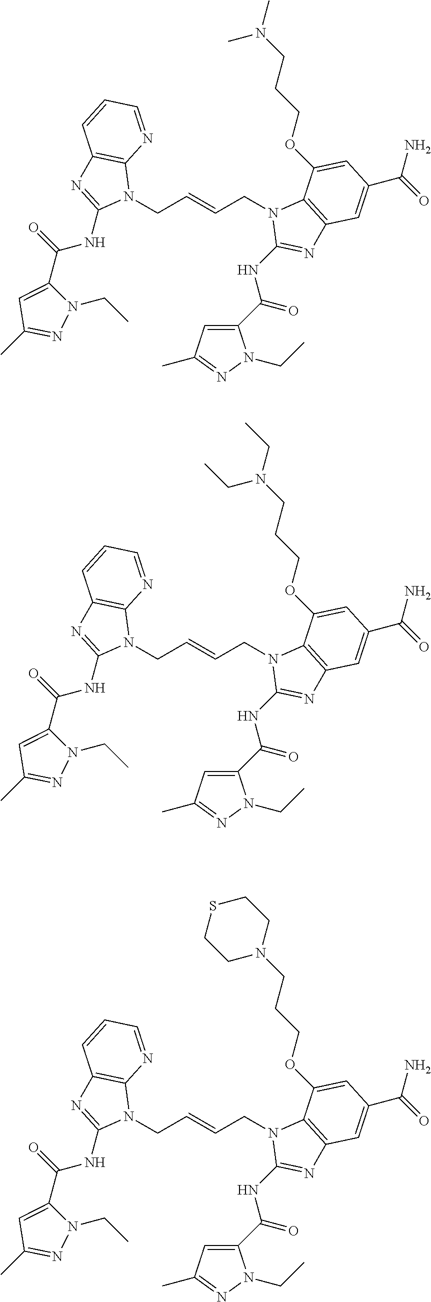 [SMILES: CCn1nc(C)cc1C(=O)Nc1nc2cccnc2n1C/C=C/Cn1c(NC(=O)c2cc(C)nn2CC)nc2cc(C(N)=O)cc(OCCCN(C)C)c21 ; CCN(CC)CCCOc1cc(C(N)=O)cc2nc(NC(=O)c3cc(C)nn3CC)n(C/C=C/Cn3c(NC(=O)c4cc(C)nn4CC)nc4cccnc43)c12 ; CCn1nc(C)cc1C(=O)Nc1nc2cccnc2n1C/C=C/Cn1c(NC(=O)c2cc(C)nn2CC)nc2cc(C(N)=O)cc(OCCCN3CCSCC3)c21]

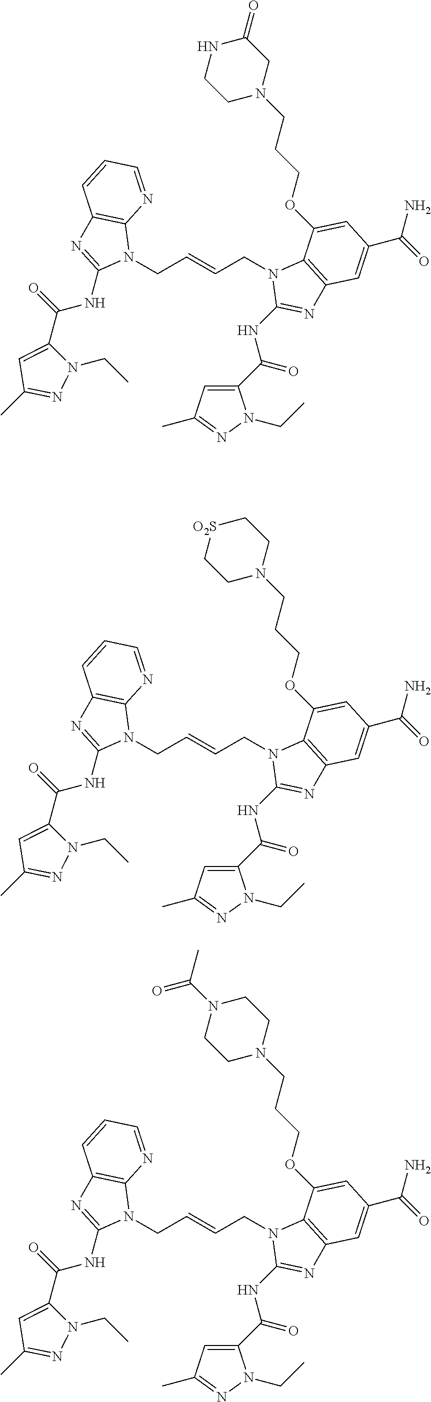

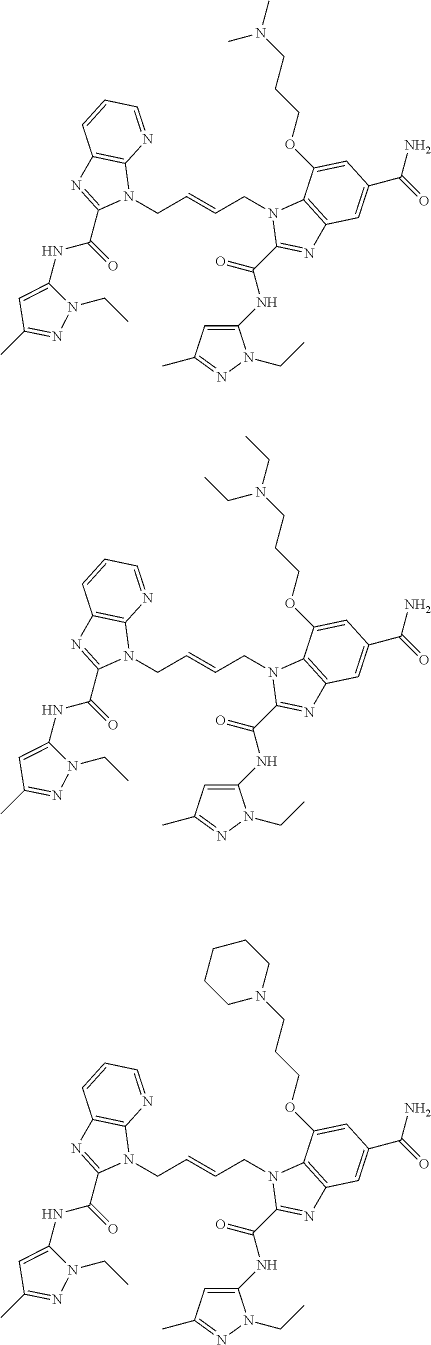 [SMILES: CCn1nc(C)cc1NC(=O)c1nc2cccnc2n1C/C=C/Cn1c(C(=O)Nc2cc(C)nn2CC)nc2cc(C(N)=O)cc(OCCCN(C)C)c21 ; CCN(CC)CCCOc1cc(C(N)=O)cc2nc(C(=O)Nc3cc(C)nn3CC)n(C/C=C/Cn3c(C(=O)Nc4cc(C)nn4CC)nc4cccnc43)c12 ; CCn1nc(C)cc1NC(=O)c1nc2cccnc2n1C/C=C/Cn1c(C(=O)Nc2cc(C)nn2CC)nc2cc(C(N)=O)cc(OCCCN3CCCCC3)c21]

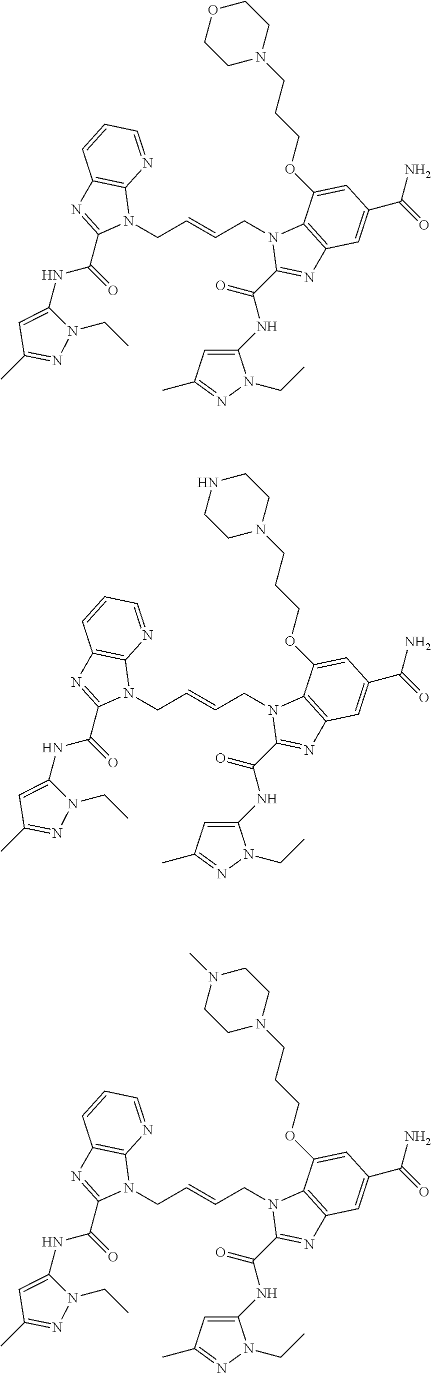 [SMILES: CCn1nc(C)cc1NC(=O)c1nc2cccnc2n1C/C=C/Cn1c(C(=O)Nc2cc(C)nn2CC)nc2cc(C(N)=O)cc(OCCCN3CCOCC3)c21 ; CCn1nc(C)cc1NC(=O)c1nc2cccnc2n1C/C=C/Cn1c(C(=O)Nc2cc(C)nn2CC)nc2cc(C(N)=O)cc(OCCCN3CCNCC3)c21 ; CCn1nc(C)cc1NC(=O)c1nc2cccnc2n1C/C=C/Cn1c(C(=O)Nc2cc(C)nn2CC)nc2cc(C(N)=O)cc(OCCCN3CCN(C)CC3)c21]

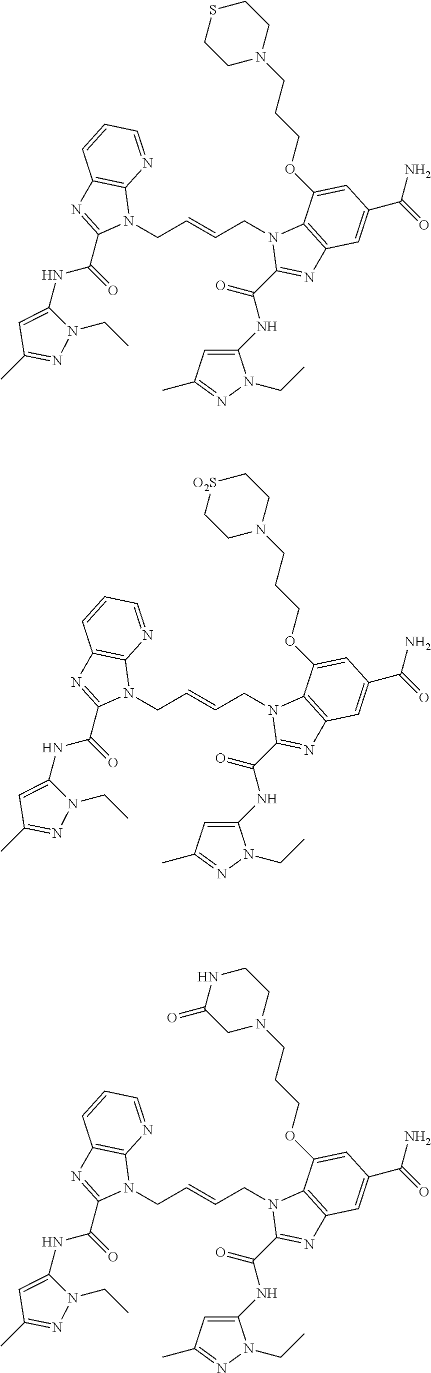 [SMILES: CCn1nc(C)cc1NC(=O)c1nc2cccnc2n1C/C=C/Cn1c(C(=O)Nc2cc(C)nn2CC)nc2cc(C(N)=O)cc(OCCCN3CCSCC3)c21 ; CCn1nc(C)cc1NC(=O)c1nc2cccnc2n1C/C=C/Cn1c(C(=O)Nc2cc(C)nn2CC)nc2cc(C(N)=O)cc(OCCCN3CCS(=O)(=O)CC3)c21 ; CCn1nc(C)cc1NC(=O)c1nc2cccnc2n1C/C=C/Cn1c(C(=O)Nc2cc(C)nn2CC)nc2cc(C(N)=O)cc(OCCCN3CCNC(=O)C3)c21]

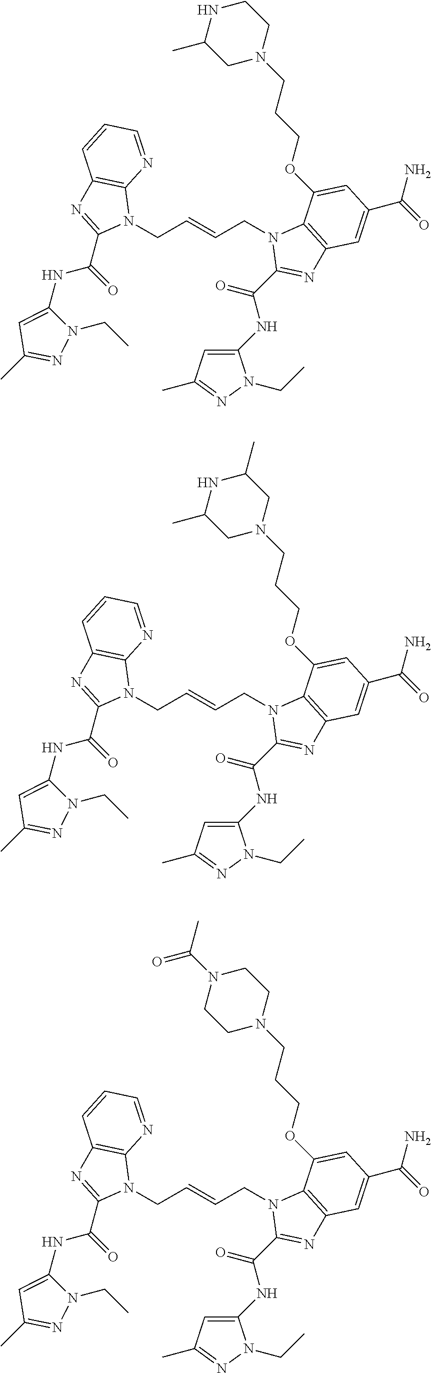 [SMILES: CCn1nc(C)cc1NC(=O)c1nc2cccnc2n1C/C=C/Cn1c(C(=O)Nc2cc(C)nn2CC)nc2cc(C(N)=O)cc(OCCCN3CCNC(C)C3)c21 ; CCn1nc(C)cc1NC(=O)c1nc2cccnc2n1C/C=C/Cn1c(C(=O)Nc2cc(C)nn2CC)nc2cc(C(N)=O)cc(OCCCN3CC(C)NC(C)C3)c21 ; CCn1nc(C)cc1NC(=O)c1nc2cccnc2n1C/C=C/Cn1c(C(=O)Nc2cc(C)nn2CC)nc2cc(C(N)=O)cc(OCCCN3CCN(C(C)=O)CC3)c21]

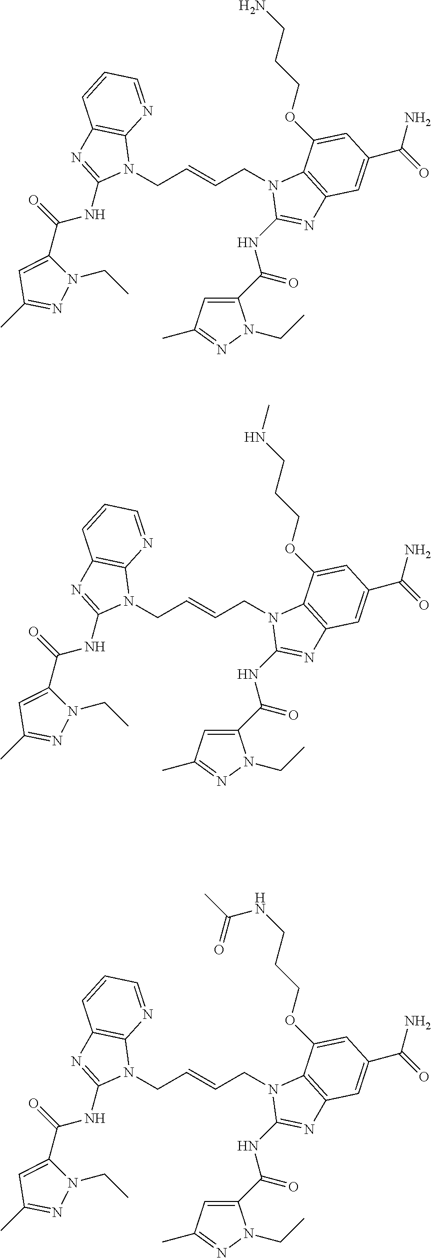 [SMILES: CCn1nc(C)cc1C(=O)Nc1nc2cccnc2n1C/C=C/Cn1c(NC(=O)c2cc(C)nn2CC)nc2cc(C(N)=O)cc(OCCCN)c21 ; CCn1nc(C)cc1C(=O)Nc1nc2cccnc2n1C/C=C/Cn1c(NC(=O)c2cc(C)nn2CC)nc2cc(C(N)=O)cc(OCCCNC)c21 ; CCn1nc(C)cc1C(=O)Nc1nc2cccnc2n1C/C=C/Cn1c(NC(=O)c2cc(C)nn2CC)nc2cc(C(N)=O)cc(OCCCNC(C)=O)c21]

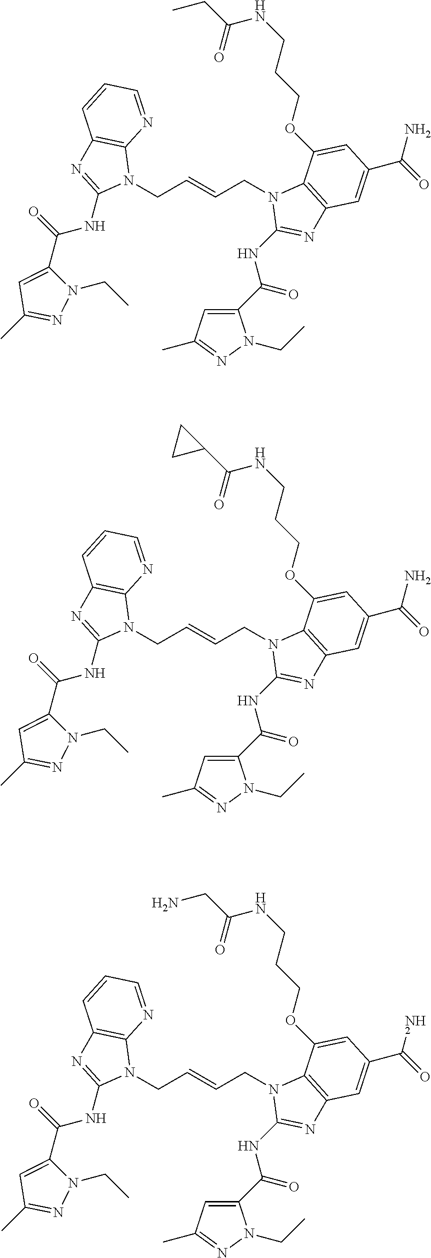 [SMILES: CCC(=O)NCCCOc1cc(C(N)=O)cc2nc(NC(=O)c3cc(C)nn3CC)n(C/C=C/Cn3c(NC(=O)c4cc(C)nn4CC)nc4cccnc43)c12 ; CCn1nc(C)cc1C(=O)Nc1nc2cccnc2n1C/C=C/Cn1c(NC(=O)c2cc(C)nn2CC)nc2cc(C(N)=O)cc(OCCCNC(=O)C3CC3)c21 ; CCn1nc(C)cc1C(=O)Nc1nc2cccnc2n1C/C=C/Cn1c(NC(=O)c2cc(C)nn2CC)nc2cc(C(N)=O)cc(OCCCNC(=O)CN)c21]

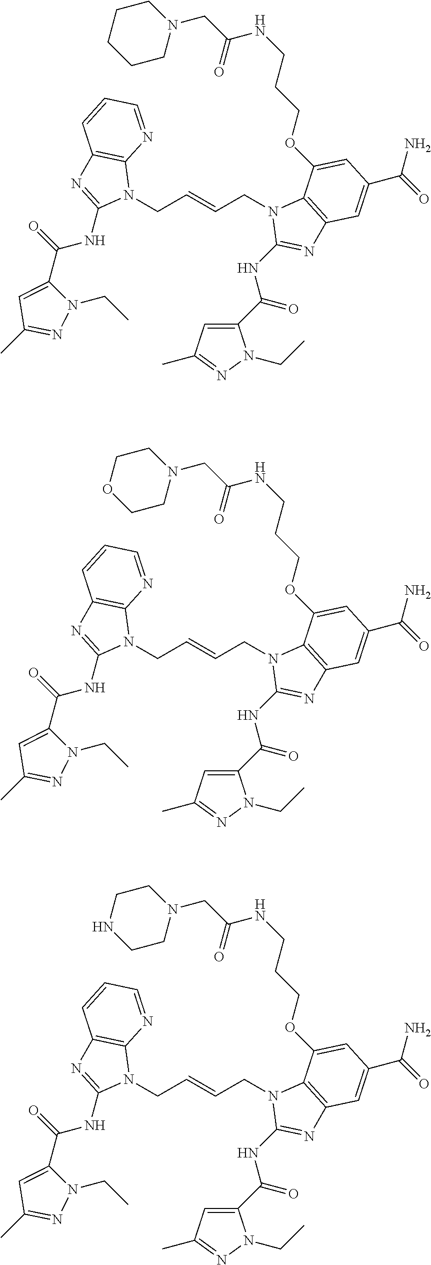 [SMILES: CCn1nc(C)cc1C(=O)Nc1nc2cccnc2n1C/C=C/Cn1c(NC(=O)c2cc(C)nn2CC)nc2cc(C(N)=O)cc(OCCCNC(=O)CN3CCCCC3)c21 ; CCn1nc(C)cc1C(=O)Nc1nc2cccnc2n1C/C=C/Cn1c(NC(=O)c2cc(C)nn2CC)nc2cc(C(N)=O)cc(OCCCNC(=O)CN3CCOCC3)c21 ; CCn1nc(C)cc1C(=O)Nc1nc2cccnc2n1C/C=C/Cn1c(NC(=O)c2cc(C)nn2CC)nc2cc(C(N)=O)cc(OCCCNC(=O)CN3CCNCC3)c21]

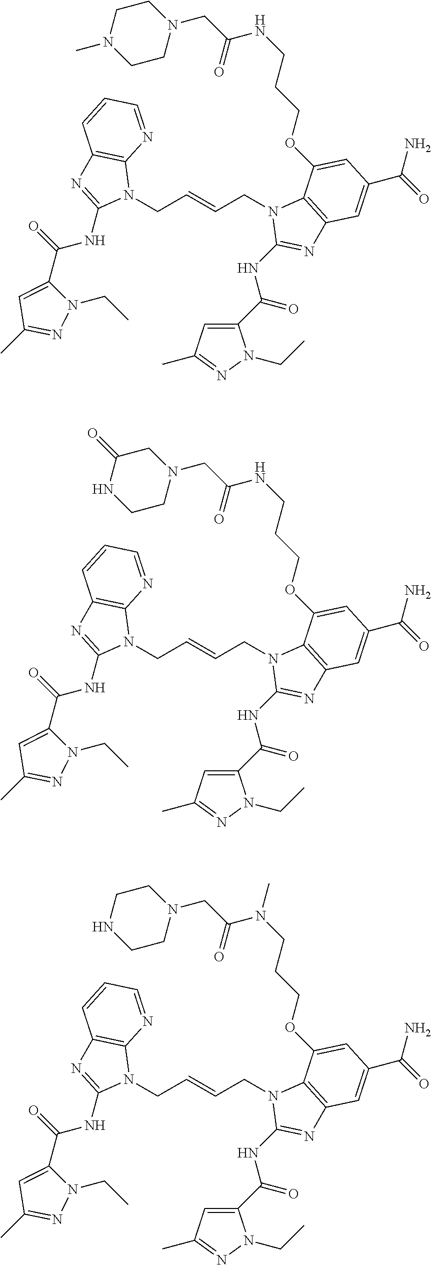 [SMILES: CCn1nc(C)cc1C(=O)Nc1nc2cccnc2n1C/C=C/Cn1c(NC(=O)c2cc(C)nn2CC)nc2cc(C(N)=O)cc(OCCCNC(=O)CN3CCN(C)CC3)c21 ; CCn1nc(C)cc1C(=O)Nc1nc2cccnc2n1C/C=C/Cn1c(NC(=O)c2cc(C)nn2CC)nc2cc(C(N)=O)cc(OCCCNC(=O)CN3CCNC(=O)C3)c21 ; CCn1nc(C)cc1C(=O)Nc1nc2cccnc2n1C/C=C/Cn1c(NC(=O)c2cc(C)nn2CC)nc2cc(C(N)=O)cc(OCCCN(C)C(=O)CN3CCNCC3)c21]

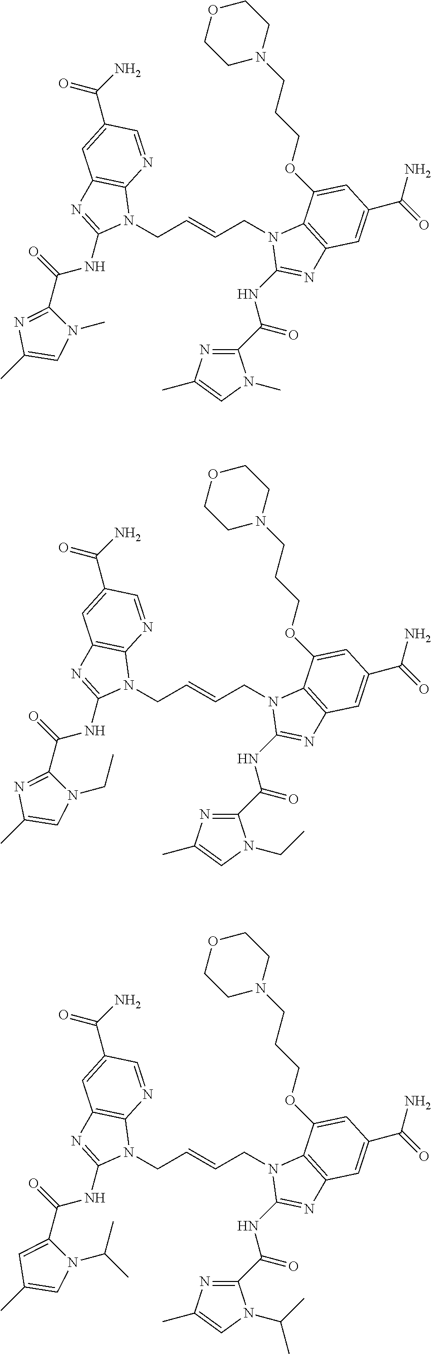 [SMILES: Cc1cn(C)c(C(=O)Nc2nc3cc(C(N)=O)cnc3n2C/C=C/Cn2c(NC(=O)c3nc(C)cn3C)nc3cc(C(N)=O)cc(OCCCN4CCOCC4)c32)n1 ; CCn1cc(C)nc1C(=O)Nc1nc2cc(C(N)=O)cnc2n1C/C=C/Cn1c(NC(=O)c2nc(C)cn2CC)nc2cc(C(N)=O)cc(OCCCN3CCOCC3)c21 ; Cc1cc(C(=O)Nc2nc3cc(C(N)=O)cnc3n2C/C=C/Cn2c(NC(=O)c3nc(C)cn3C(C)C)nc3cc(C(N)=O)cc(OCCCN4CCOCC4)c32)n(C(C)C)c1]

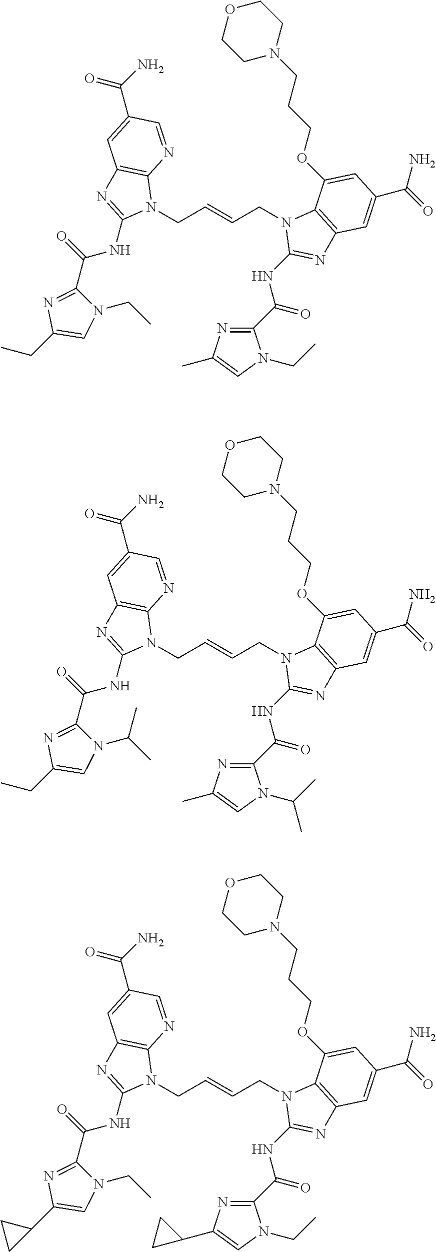 [SMILES: CCc1cn(CC)c(C(=O)Nc2nc3cc(C(N)=O)cnc3n2C/C=C/Cn2c(NC(=O)c3nc(C)cn3CC)nc3cc(C(N)=O)cc(OCCCN4CCOCC4)c32)n1 ; CCc1cn(C(C)C)c(C(=O)Nc2nc3cc(C(N)=O)cnc3n2C/C=C/Cn2c(NC(=O)c3nc(C)cn3C(C)C)nc3cc(C(N)=O)cc(OCCCN4CCOCC4)c32)n1 ; CCn1cc(C2CC2)nc1C(=O)Nc1nc2cc(C(N)=O)cnc2n1C/C=C/Cn1c(NC(=O)c2nc(C3CC3)cn2CC)nc2cc(C(N)=O)cc(OCCCN3CCOCC3)c21]

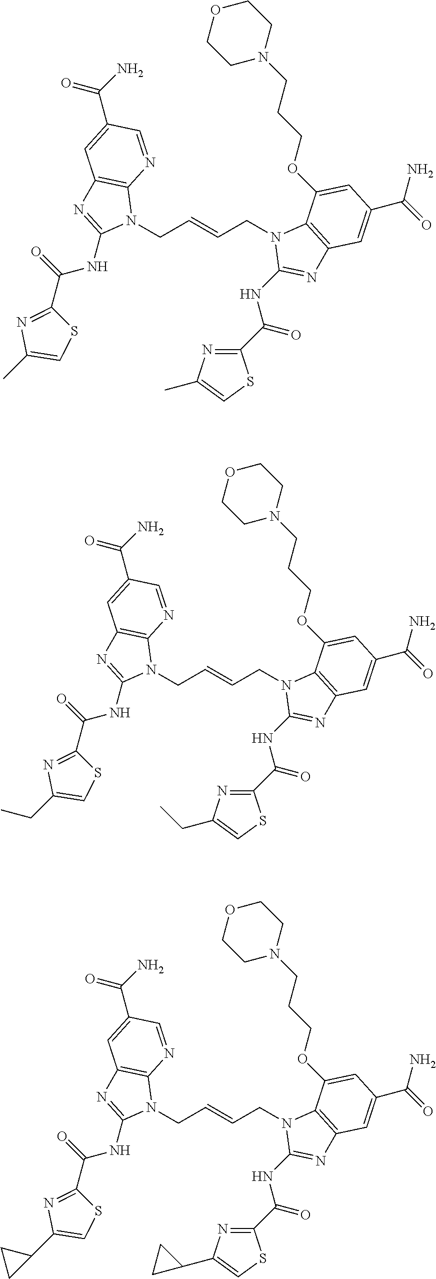 [SMILES: Cc1csc(C(=O)Nc2nc3cc(C(N)=O)cnc3n2C/C=C/Cn2c(NC(=O)c3nc(C)cs3)nc3cc(C(N)=O)cc(OCCCN4CCOCC4)c32)n1 ; CCc1csc(C(=O)Nc2nc3cc(C(N)=O)cnc3n2C/C=C/Cn2c(NC(=O)c3nc(CC)cs3)nc3cc(C(N)=O)cc(OCCCN4CCOCC4)c32)n1 ; NC(=O)c1cnc2c(c1)nc(NC(=O)c1nc(C3CC3)cs1)n2C/C=C/Cn1c(NC(=O)c2nc(C3CC3)cs2)nc2cc(C(N)=O)cc(OCCCN3CCOCC3)c21]

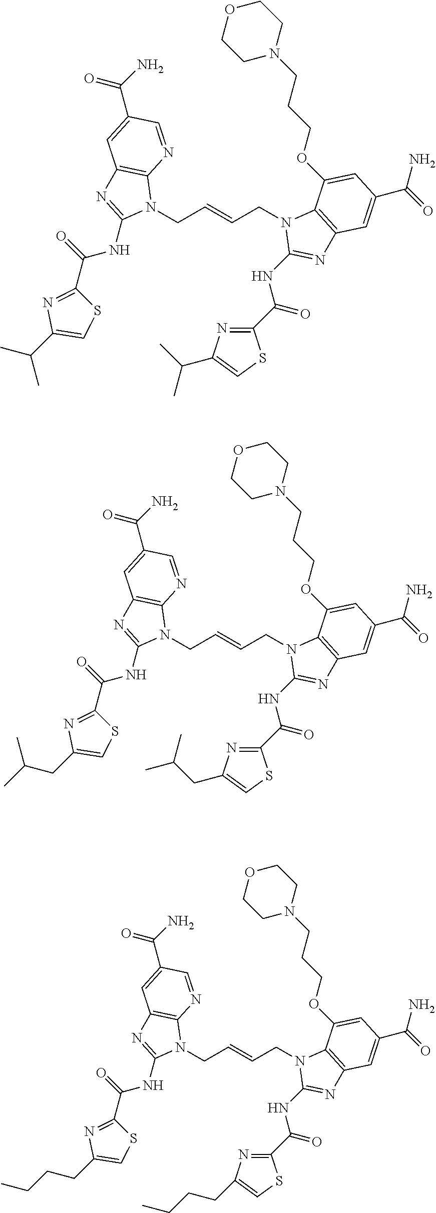 [SMILES: CC(C)c1csc(C(=O)Nc2nc3cc(C(N)=O)cnc3n2C/C=C/Cn2c(NC(=O)c3nc(C(C)C)cs3)nc3cc(C(N)=O)cc(OCCCN4CCOCC4)c32)n1 ; CC(C)Cc1csc(C(=O)Nc2nc3cc(C(N)=O)cnc3n2C/C=C/Cn2c(NC(=O)c3nc(CC(C)C)cs3)nc3cc(C(N)=O)cc(OCCCN4CCOCC4)c32)n1 ; CCCCc1csc(C(=O)Nc2nc3cc(C(N)=O)cnc3n2C/C=C/Cn2c(NC(=O)c3nc(CCCC)cs3)nc3cc(C(N)=O)cc(OCCCN4CCOCC4)c32)n1]

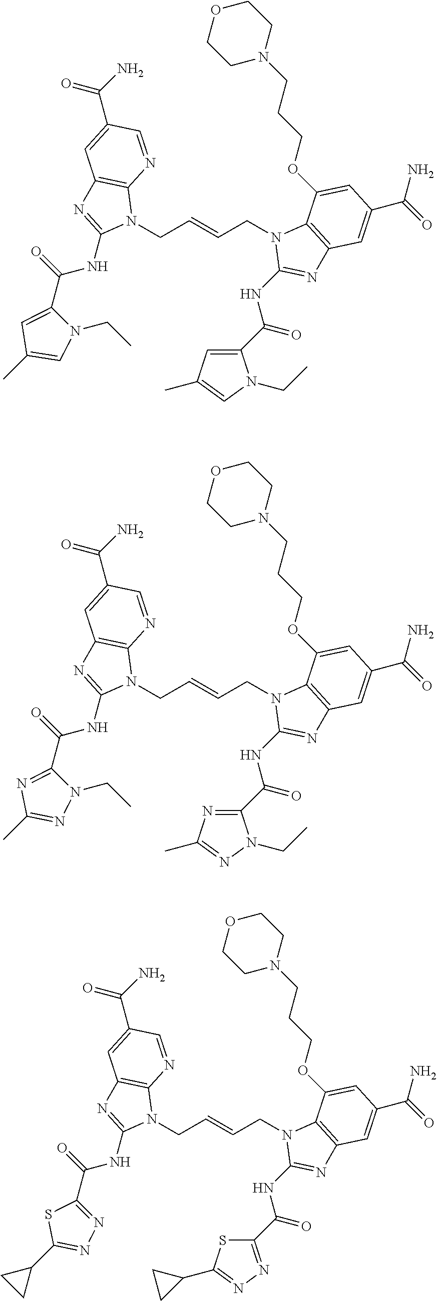 [SMILES: CCn1cc(C)cc1C(=O)Nc1nc2cc(C(N)=O)cnc2n1C/C=C/Cn1c(NC(=O)c2cc(C)cn2CC)nc2cc(C(N)=O)cc(OCCCN3CCOCC3)c21 ; CCn1nc(C)nc1C(=O)Nc1nc2cc(C(N)=O)cnc2n1C/C=C/Cn1c(NC(=O)c2nc(C)nn2CC)nc2cc(C(N)=O)cc(OCCCN3CCOCC3)c21 ; NC(=O)c1cnc2c(c1)nc(NC(=O)c1nnc(C3CC3)s1)n2C/C=C/Cn1c(NC(=O)c2nnc(C3CC3)s2)nc2cc(C(N)=O)cc(OCCCN3CCOCC3)c21]

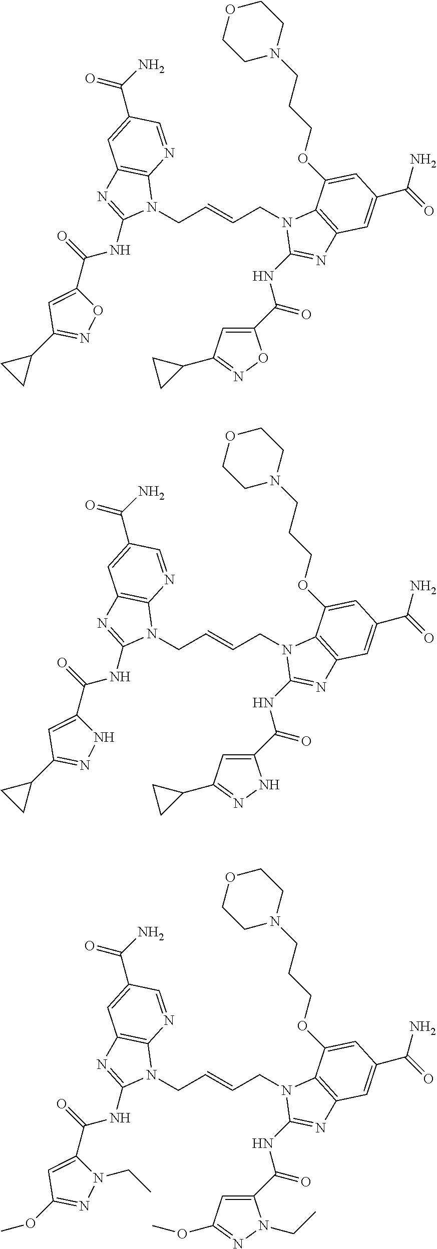 [SMILES: NC(=O)c1cnc2c(c1)nc(NC(=O)c1cc(C3CC3)no1)n2C/C=C/Cn1c(NC(=O)c2cc(C3CC3)no2)nc2cc(C(N)=O)cc(OCCCN3CCOCC3)c21 ; NC(=O)c1cnc2c(c1)nc(NC(=O)c1cc(C3CC3)n[nH]1)n2C/C=C/Cn1c(NC(=O)c2cc(C3CC3)n[nH]2)nc2cc(C(N)=O)cc(OCCCN3CCOCC3)c21 ; CCn1nc(OC)cc1C(=O)Nc1nc2cc(C(N)=O)cnc2n1C/C=C/Cn1c(NC(=O)c2cc(OC)nn2CC)nc2cc(C(N)=O)cc(OCCCN3CCOCC3)c21]

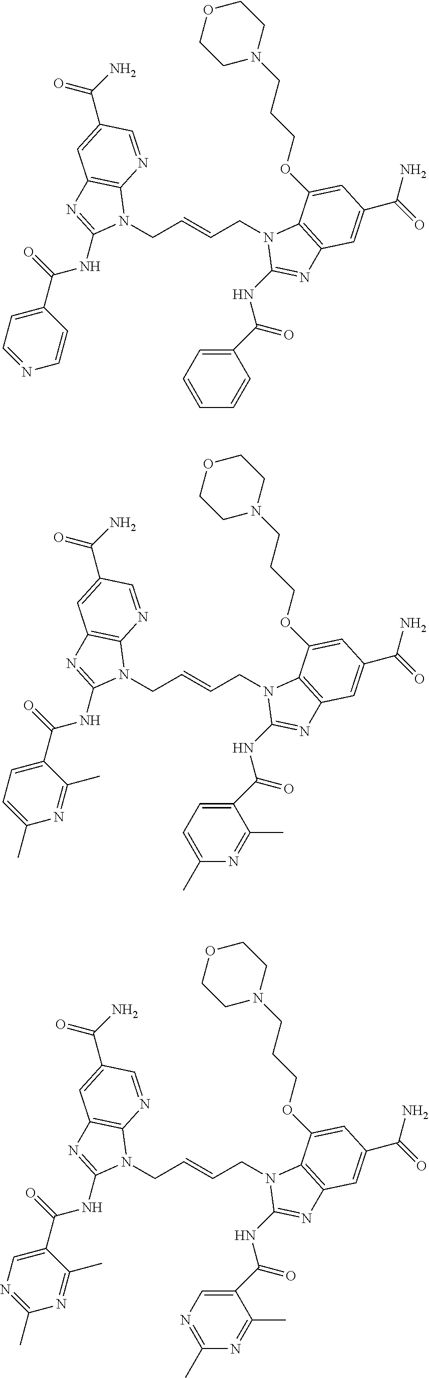 [SMILES: NC(=O)c1cnc2c(c1)nc(NC(=O)c1ccncc1)n2C/C=C/Cn1c(NC(=O)c2ccccc2)nc2cc(C(N)=O)cc(OCCCN3CCOCC3)c21 ; Cc1ccc(C(=O)Nc2nc3cc(C(N)=O)cnc3n2C/C=C/Cn2c(NC(=O)c3ccc(C)nc3C)nc3cc(C(N)=O)cc(OCCCN4CCOCC4)c32)c(C)n1 ; Cc1ncc(C(=O)Nc2nc3cc(C(N)=O)cnc3n2C/C=C/Cn2c(NC(=O)c3cnc(C)nc3C)nc3cc(C(N)=O)cc(OCCCN4CCOCC4)c32)c(C)n1]

The compounds of this invention may contain one or more asymmetric centers and may be present as racemic mixtures, diastereomeric mixtures, enantiomerically enriched mixtures, diastereomerically enriched mixtures, pharmaceutically acceptable salts, tautomer mixtures, and enantiomerically or diastereomerically pure individual stereoisomers.

The stereochemistry of the chiral center present in compounds of this invention is generally represented in the compound names and/or in the chemical structures illustrated herein. Where the stereochemistry of a chiral center present in a compound of this invention, or in any chemical structure illustrated herein, is not specified, the structure is intended to encompass any stereoisomer and all mixtures thereof. Accordingly, the present invention encompasses all isomers of the compounds of Formula (I), (Ia), (Ib), (Ic) or (Id), and salts thereof, whether as individual isomers isolated such as to be substantially free of the other isomer (i.e. pure) or as mixtures (i.e. racemates and racemic mixtures and tautomers).

Individual stereoisomers of a compound of this invention may be resolved (or mixtures of stereoisomers may be enriched) using methods known to those skilled in the art. It will be appreciated that where the desired stereoisomer is converted into another chemical entity by one of the separation procedures described above, a further step is required to liberate the desired form.

Some of the compounds of the present invention are capable of forming salts with various inorganic and organic acids and bases and such salts are also within the scope of this invention. Examples of such acid addition salts include acetate, adipate, ascorbate, benzoate, benzenesulfonate, bicarbonate, bisulfate, butyrate, camphorate, camphorsulfonate, choline, citrate, cyclohexyl sulfamate, diethylenediamine, ethanesulfonate, fumarate, glutamate, glycolate, hemisulfate, 2-hydroxyethylsulfonate, heptanoate, hexanoate, hydrochloride, hydrobromide, hydroiodide, hydroxymaleate, lactate, malate, maleate, methanesulfonate, meglumine, 2-naphthalenesurfonate, nitrate, oxalate, pamoate, persulfate, phenylacetate, phosphate, diphosphate, picrate, pivalate, propionate, quinate, salicylate, stearate, succinate, sulfamate, sulfanilate, sulfate, tartrate, tosylate (p-toluenesulfonate), trifluoroacetate, and undecanoate. Base salts include ammonium salts, alkali metal salts such as sodium, lithium and potassium salts, alkaline earth metal salts such as aluminum, calcium and magnesium salts, salts with organic bases such as dicyclohexylamine salts, N-methyl-D-glucamine, and salts with amino acids such as arginine, lysine, ornithine, and so forth. Also, basic nitrogen-containing groups may be quaternized with such agents as: lower alkyl halides, such as methyl, ethyl, propyl, and butyl halides; dialkyl sulfates like dimethyl, diethyl, dibutyl; diamyl sulfates; long chain halides such as decyl, lauryl, myristyl and stearyl halides; aralkyl halides like benzyl bromide and others. Non-toxic physiologically-acceptable salts are preferred, although other salts are also useful, such as in isolating or purifying the product.

The salts may be formed by conventional means, such as by reacting the free base form of the product with one or more equivalents of the appropriate acid in a solvent or medium in which the salt is insoluble, or in a solvent such as water, which is removed in vacuo or by freeze drying or by exchanging the anions of an existing salt for another anion on a suitable ion-exchange resin.

When a disclosed compound or its salt is named or depicted by structure, it is to be understood that the compound or salt, including solvates (particularly, hydrates) thereof, may exist in crystalline forms, non-crystalline forms or a mixture thereof. The compound or salt, or solvates (particularly, hydrates) thereof, may also exhibit polymorphism (i.e. the capacity to occur in different crystalline forms). These different crystalline forms are typically known as “polymorphs.” It is to be understood that the invention includes all polymorphs of any compound of this invention, e.g., all polymorphic forms of any compound named or depicted by structure herein, including any salts and/or solvates (particularly, hydrates) thereof.

Polymorphs have the same chemical composition but differ in packing, geometrical arrangement, and other descriptive properties of the crystalline solid state. Polymorphs, therefore, may have different physical properties such as shape, density, deformability, stability, and dissolution properties. Polymorphs typically exhibit different melting points, IR spectra, and X-ray powder diffraction patterns, which may be used for identification. It will be appreciated that different polymorphs may be produced, for example, by changing or adjusting the conditions used in crystallizing/recrystallizing the compound. Polymorphic forms may be characterized and differentiated using a number of conventional analytical techniques, including, but not limited to, X-ray powder diffraction (XRPD) patterns, infrared (IR) spectra, raman spectra, differential scanning calorimetry (DSC), thermogravimetric analysis (TGA) and solid state nuclear magnetic resonance (SSNMR).

The skilled artisan will appreciate that pharmaceutically acceptable solvates (particularly, hydrates) of a compound of Formula (I), including pharmaceutically acceptable solvates of a pharmaceutically acceptable salt of a compound of Formula (I), may be formed when solvent molecules are incorporated into the crystalline lattice during crystallization. Solvates may involve non-aqueous solvents such as ethanol, or they may involve water as the solvent that is incorporated into the crystalline lattice. Solvates wherein water is the solvent that is incorporated into the crystalline lattice are typically referred to as “hydrates.” The present invention includes within its scope all possible stoichiometric and non-stoichiometric salt and/or hydrate forms.

The invention encompasses all prodrugs of the compounds of this invention, which upon administration to the recipient are capable of providing (directly or indirectly) a compound of this invention, or an active metabolite or residue thereof.

It is to be further understood that the present invention includes within its scope all tautomeric or isomer forms of any free base form of the compounds of this invention as well as all possible stoichiometric and non-stoichiometric salt forms.

### Compositions and Therapeutic Methods

In an aspect is provided a pharmaceutical composition including a compound described herein and a pharmaceutically acceptable excipient.

In embodiments, the pharmaceutical composition includes an effective amount of the compound. In embodiments, the pharmaceutical composition includes a therapeutically effective amount of the compound. In embodiments, the pharmaceutical composition includes a second agent. In embodiments, the pharmaceutical composition includes a therapeutically effective amount of a second agent. In embodiments, the second agent is an anti-cancer agent. In embodiments, the second agent is an agent for treating a viral infection, hyperproliferative disease or cancer.

In an aspect is provided a method of treating cancer in a subject in need of such treatment, including administering to the subject an effective amount of a compound described herein. In embodiments, the method includes an effective amount of the compound. In embodiments, the method includes a therapeutically effective amount of the compound.

In an aspect is provided a method of treating a viral infection in a subject in need of such treatment, including administering to the subject an effective amount of a compound described herein. In embodiments, the method includes an effective amount of the compound. In embodiments, the method includes a therapeutically effective amount of the compound.

In embodiments, the method includes an effective amount of the compound. In embodiments, the method includes a therapeutically effective amount of the compound. In embodiments, the method includes administering a second agent. In embodiments, the method includes administering a therapeutically effective amount of a second agent. In embodiments, the second agent is an anti-cancer agent. In embodiments, the second agent is an agent for treating a viral infection, hyperproliferative disease or cancer.

The pharmaceutical compositions provided herein can be co-formulated with other active ingredients which do not impair the desired therapeutic action, or with substances that supplement the desired action.

In one embodiment, the pharmaceutical compositions are provided in a dosage form for parenteral administration, which comprise a compound provided herein, e.g., a compound of Formula I, or a pharmaceutically acceptable salt, solvate or hydrate thereof, and one or more pharmaceutically acceptable excipients or carriers.

The compositions for parenteral administration can be utilized for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

In another embodiment, the pharmaceutical compositions are provided in a dosage form for oral administration in solid, semisolid, or liquid dosage forms. As used herein, oral administration also includes buccal, lingual, and sublingual administration. Suitable oral dosage forms include, but are not limited to, tablets, fast melts, chewable tablets, capsules, pills, troches, lozenges, pastilles, cachets, pellets, medicated chewing gum, bulk powders, effervescent or non-effervescent powders or granules, solutions, emulsions, suspensions, wafers, sprinkles, elixirs, and syrups. In addition to the active ingredient(s), the pharmaceutical compositions can contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

The pharmaceutical compositions provided herein can be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenyl salicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms can be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein can be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides.

The capsules may also be coated as known by those of skill in the art in order to modify or sustain dissolution of the active ingredient.

In another embodiment, the pharmaceutical compositions provided herein can be administered topically to the skin, orifices, or mucosa. The topical administration, as used herein, includes (intra)dermal, conjunctival, intracorneal, intraocular, ophthalmic, auricular, transdermal, nasal, vaginal, urethral, respiratory, and rectal administration.

The pharmaceutical compositions provided herein can be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, dermal patches. The topical formulation of the pharmaceutical compositions provided herein can also comprise liposomes, micelles, microspheres, nanosystems, and mixtures thereof.

In another embodiment, the pharmaceutical compositions provided herein can be administered intranasally or by inhalation to the respiratory tract. The pharmaceutical compositions can be provided in the form of an aerosol or solution for delivery using a pressurized container, pump, spray, atomizer, such as an atomizer using electrohydrodynamics to produce a fine mist, or nebulizer, alone or in combination with a suitable propellant, such as 1, 1, 1,2-tetrafluoroethane or 1, 1, 1,2,3,3,3-heptafluoropropane. The pharmaceutical compositions can also be provided as a dry powder for insufflation, alone or in combination with an inert carrier such as lactose or phospholipids; and nasal drops. For intranasal use, the powder can comprise a bioadhesive agent, including chitosan or cyclodextrin.

The pharmaceutical compositions provided herein can be micronized to a size suitable for delivery by inhalation, such as about 50 micrometers or less, or about 10 micrometers or less. Particles of such sizes can be prepared using a comminuting method known to those skilled in the art, such as spiral jet milling, fluid bed jet milling, supercritical fluid processing to form nanoparticles, high pressure homogenization, or spray drying.

In addition to the above described routes of administration suitable for treatment of oncology, the pharmaceutical compositions may be adapted for administration by intratumoral or peritumoral injection. The intratumoral or peritumoral injection of a compound of the present invention directly into or adjacent to a single solid tumor is expected to elicit an immune response that can attack and destroy cancer cells throughout the body, substantially reducing and in some cases permanently eliminating the tumor from the diseased subject.

The pharmaceutical compositions provided herein can be formulated as a modified release dosage form. As used herein, the term “modified release” refers to a dosage form in which the rate or place of release of the active ingredient(s) is different from that of an immediate dosage form when administered by the same route. Modified release dosage forms include delayed-, extended-, prolonged-, sustained-, pulsatile-, controlled-, accelerated- and fast-, targeted-, programmed-release, and gastric retention dosage forms. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphorism of the active ingredient(s).

The pharmaceutical compositions of the invention typically contain one compound of the invention. However, in certain embodiments, the pharmaceutical compositions of the invention contain more than one compound of the invention. In addition, the pharmaceutical compositions of the invention may optionally further comprise one or more additional therapeutic agents, (e.g., pharmaceutically active compounds).

It will be understood that the compounds of this invention may also be formulated with vaccines as adjuvants to modulate their activity. Such compositions may contain antibody (antibodies) or antibody fragment(s) or an antigenic component including but not limited to protein, DNA, live or dead bacteria and/or whole, inactivated or split viruses or virus-like particles, recombinant proteins or antigenic fragments thereof, optionally together with one or more other components with adjuvant activity including but not limited to aluminum salts, oil and water emulsions, heat shock proteins, saponins, lipid A preparations and derivatives, glycolipids, liposomes, TLR agonists such as CpG DNA or similar agents, cytokines such as GM-CSF or IL-12, or similar agents.

The compounds of the invention are useful in the treatment or prevention of diseases and disorders in which modulation of STING is beneficial. Such STING mediated diseases and disorders include inflammation, allergic and autoimmune diseases, infectious diseases, cancer and precancerous syndromes. The compounds of the invention are also useful as an immugenic composition or vaccine adjuvant. Accordingly, this invention is directed to a method of modulating STING comprising contacting a cell with a compound of the invention.

One aspect of the invention provides methods of treatment or prevention of STING mediated diseases and disorders, in which agonizing STING is beneficial. Exemplary diseases/disorders include, but are not limited to, cancer, infectious disease (e.g., HIV, HBV, HCV, HPV, and influenza), vaccine adjuvant.

In certain embodiments, provided herein are methods of using the disclosed compounds and compositions, or pharmaceutically acceptable salts, solvates or hydrates thereof, for the treatment, prevention, or amelioration of a disease or disorder selected from myeloproliferative disorders such as polycythemia vera (PCV), essential thrombocythemia and idiopathic myelofibrosis (IMF) and hypereosinophilic syndrome (HES); leukemia such as myeloid leukemia including chronic myeloid leukemia (CML), imatinib-resistant forms of CML, acute myeloid leukemia (AML), acute lymphoblastic leukemia (ALL) and a subtype of AML, acute megakaryoblastic leukemia (AMKL); lymphoproliferative diseases such as myeloma; cancer including head and neck cancer, prostate cancer, breast cancer, ovarian cancer, melanoma, lung cancer, brain cancer, pancreatic cancer, gastric cancer, thyroid cancer, renal carcinoma, Kaposi's sarcoma, Castleman's disease, melanoma; and inflammatory diseases or disorders related to immune dysfunction, immunodeficiency or immunomodulation, such as tissue transplant rejection, graft-versus-host disease, wound healing, kidney disease including diabetic neuropathy; autoimmune diseases such as multiple sclerosis, thyroiditis, type 1 diabetes, sarcoidosis, psoriasis, allergic rhinitis, atopic dermatitis, myasthenia gravis, inflammatory bowel disease including Crohn's disease and ulcerative colitis (UC), systemic lupus erythematosus (SLE), arthritis, osteoarthritis, rheumatoid arthritis, osteoporosis, asthma and chronic obstructive pulmonary disease (COPD), inflammatory diseases of the eye including conjunctivitis, uveitis, iritis, scleritis, inflammatory diseases of the respiratory tract including the upper respiratory tract such as rhinitis and sinusitis and inflammatory diseases of the lower respiratory tract including bronchitis; inflammatory myopathy such as myocarditis, other inflammatory diseases such as ischemia reperfusion injuries related to an inflammatory ischemic event such as a stroke or cardiac arrest, and other inflammatory conditions such as systemic inflammatory response syndrome (SIRS) and sepsis.

The compounds of this invention may be employed alone or in combination with other therapeutic agents. As modulators of the immune response, the compounds of this invention may also be used in monotherapy or used in combination with another therapeutic agent in the treatment of diseases and conditions in which modulation of STING is beneficial. Combination therapies according to the present invention thus comprise the administration of a compound of Formula (I) or a pharmaceutically acceptable salt thereof, and at least one other therapeutically active agent.

In one embodiment, combination therapies according to the present invention comprise the administration of at least one compound of Formula (I) or a pharmaceutically acceptable salt thereof, and at least one other therapeutic agent. The compound(s) of Formula (I) and pharmaceutically acceptable salts thereof, and the other therapeutic agent(s) may be administered together in a single pharmaceutical composition or separately and, when administered separately this may occur simultaneously or sequentially in any order. The amounts of the compound(s) of Formula (I) and pharmaceutically acceptable salts thereof, and the other therapeutic agent(s) and the relative timings of administration will be selected in order to achieve the desired combined therapeutic effect. Thus, in a further aspect, there is provided a combination comprising a compound of Formula (I), or a pharmaceutically acceptable salt thereof, together with one or more other therapeutic agents, such as anti-cancer agents.

In one embodiment, the combination of the present invention comprises a compound of Formula (I), or a salt thereof, particularly a pharmaceutically acceptable salt thereof, and at least one anti-cancer agent selected from the anti-metabolites (e.g., 5-fluoro-uracil, cytarabine, methotrexate, fludarabine and others), antimicrotubule agents (e.g., vinca alkaloids such as vincristine, vinblastine; taxanes such as paclitaxel and docetaxel), alkylating agents (e.g., cyclophosphamide, melphalan, carmustine, nitrosoureas such as bischloroethylnitrosurea and hydroxyurea), platinum agents (e.g. cisplatin, carboplatin, oxaliplatin and satraplatin), anthracyclines (e.g., doxrubicin and daunorubicin), antitumor antibiotics (e.g., mitomycin, idarubicin, adriamycin and daunomycin), topoisomerase inhibitors (e.g., etoposide and camptothecins), anti-angiogenesis agents (e.g. Sutent®, sorafenib and Bevacizumab) or any other cytotoxic agents, (e.g. estramustine phosphate, prednimustine), hormones or hormone agonists, antagonists, partial agonists or partial antagonists, and kinase inhibitors.

In a further embodiment, the combination of the present invention comprises a compound of Formula (I), or a salt thereof, particularly a pharmaceutically acceptable salt thereof, and at least one anti-neoplastic agent selected from the kinase inhibitor include, for example, epidermal growth factor receptor (EGFr), platelet derived growth factor receptor (PDGFr), erbB2, erbB4, Ret, vascular endothelial growth factor receptor (VEGFr), tyrosine kinase with immunoglobulin-like and epidermal growth factor homology domains (TIE-2), insulin growth factor —I (IGFI) receptor, macrophage colony stimulating factor (cfms), BTK, cKit, cMet, fibroblast growth factor (FGF) receptors (FGFR1, FGRF2, FGRF3 and FGRF4, Trk receptors (TrkA, TrkB, and TrkC), ephrin (eph) receptors, and the RET protooncogene. Tyrosine kinases, which are not growth factor receptor kinases are termed nonreceptor tyrosine kinases. Non-receptor tyrosine kinases useful in the present invention, which are targets or potential targets of anti-cancer drugs, include cSRs, Lck, Fyn, Yes, Jak, cAbl, FAK (Focal adhesion kinase), Brutons tyrosine kinase (BTK), and Bcr-Abl. In a further embodiment, the signal transduction pathway inhibitor is an inhibitor of a serine/threonine kinase Raf, akt, or PKC-zeta. In a further embodiment, the signal transduction pathway inhibitor is an inhibitor of a serine/threonine kinase selected from the group consisting of PI3K. In a further embodiment, the signal transduction pathway inhibitor is a dual EGFr/erbB2 inhibitor. In further embodiment, cell cycle signaling inhibitor is an inhibitor of CDK2, CDK4 or CDK6.

In a further embodiment, the combination of the present invention comprises a compound of Formula (I), or a salt thereof, particularly a pharmaceutically acceptable salt thereof, and immuno-modulators. As used herein “immuno-modulators” refer to any substance including monoclonal antibodies that affects the immune system. Immuno-modulators can be used as anti-neoplastic agents for the treatment of cancer. For example, immune-modulators include, but are not limited to, anti-CTLA-4 antibodies such as ipilimumab (YERVOY) and PD-1 antagonist (anti-PD-1 antibodies, Opdivo/nivolumab and Keytruda/pembrolizumab). Other immuno-modulators include, but are not limited to, ICOS antibodies, OX-40 antibodies, PD-L1 antibodies, LAG3 antibodies, TIM-3 antibodies, 41BB antibodies and GITR antibodies.

The term of “PD-1 antagonist” means any chemical compound or biological molecule that blocks binding of PD-L1 expressed on a cancer cell to PD-1 expressed on an immune cell (T cell, B cell or NKT cell) and preferably also blocks binding of PD-L² expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any embodiments of the aspects or embodiments of the present invention in which a human individual is to be treated, the PD-1 antagonist blocks binding of human PD-L1 to human PD-1, and preferably blocks binding of both human PD-L1 and PD-L2 to human PD-1.

PD-1 antagonists useful in any of the aspects of the present invention include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody or a chimeric antibody, and may include a human constant region. In some embodiments, the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)2, scFv and Fv fragments.

In some embodiments, the PD-1 inhibitor is chosen from spartalizumab (PDR001, Novartis), nivolumab (Bristol-Myers Squibb), pembrolizumab (Merck & Co), pidilizumab (CureTech), MEDI0680 (Medimmune), cemiplimab (REGN2810, Regeneron), TSR-042 (Tesaro), PF-06801591 (Pfizer), tislelizumab (BGB-A317, Beigene), BGB-108 (Beigene), INCSHR1210 (Incyte), or AMP-224 (Amplimmune).

In some embodiments, the PD-L1 inhibitor is selected from, durvalumab, atezolizumab, avelumab, DAKO 28-8, DAKO 22C3, VENTANA SP263, VENTANA SP142, or VENTANA SP73-10.

In one embodiment methods of treating a human in need thereof are provided comprising administering a compound of Formula (I) or a salt thereof and at least one IL-15 complex (such as IL-15 SA, ALT-803 or RLI-15). The cytokine, interleukin-15 (IL-15), is a member of the four alpha-helix bundle family of lymphokines produced by many cells in the body. 1L-15 is essential for development and differentiation of natural killer (NK) and memory (m)CD8⁺ T cells.

The combinations disclosed herein can result in one or more of: anti-tumor immunity, an increase in immune cell function (e.g., one or more of CD8+ T cell proliferation, NK cell proliferation, inhibition of regulatory T cell function, an effect on the activity of multiple cell types, such as CD 8+ T cells and NK cells), and an increase in tumor infiltrating lymphocytes.

The compositions may also be administered in combination with radiotherapy, surgical therapy, immunotherapy (particularly radioimmunotherapy), gene therapy, or any other therapy known to those of ordinary skill in the art for treatment of a disease or disorder.

The compounds of the invention may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound of the invention depend on the pharmacokinetic properties of that compound, such as absorption, distribution, and half-life, which can be determined by the skilled artisan. In addition, suitable dosing regimens, including the duration such regimens are administered, for a compound of the invention depend on the disease or disorder being treated, the severity of the disease or disorder being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change. Total daily dosages range from 1 mg to 2000 mg, preferably, total daily dosages range from 1 mg to 300 mg.

As provided herein, unit dosage forms (pharmaceutical compositions) containing from 1 mg to 1000 mg of a compound of the invention may be administered one, two, three, or four times per day; preferably one, two, or three times per day; and more preferably, one or two times per day, to effect treatment of a STING-mediated disease or disorder.

Two therapies may be given in either order and may precede or follow the other treatment by intervals ranging from minutes to weeks. In embodiments where the other agents are applied separately, one would generally ensure that a significant period of time did not expire between the time of each delivery, such that the agents would still be able to exert an advantageously combined effect on the patient. In such instances, it is contemplated that one may administer both modalities within about 12-24 hrs of each other and, more preferably, within about 6-12 hrs of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

The pharmaceutical compositions provided herein can be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data.

### EXAMPLES

### Synthesis

The compounds, or pharmaceutically acceptable salts thereof, of the present invention can be prepared in a number of ways well known to one skilled in the art of organic synthesis.

The compounds, or pharmaceutically acceptable salts thereof, of the present invention can be synthesized using the methods described below, together with synthetic methods known in the art of synthetic organic chemistry, or variations thereon as appreciated by those skilled in the art. Such methods include, but are not limited to, those described below. All references cited herein are hereby incorporated in their entirety by reference.

The compounds, or pharmaceutically acceptable salts thereof, of this invention may be prepared using the reactions and techniques described herein. The reactions are performed in solvents appropriate to the reagents and materials employed and are suitable for the transformations being affected. Also, in the description of the synthetic methods described below, it is to be understood that all proposed reaction conditions, including choice of solvent, reaction atmosphere, reaction temperature, duration of the experiment and workup procedures, are chosen to be the conditions standard for that reaction, which should be readily recognized by one skilled in the art. It is understood by one skilled in the art of organic synthesis that the functionality present on various portions of the molecule must be compatible with the reagents and reactions proposed. Such restrictions to the substituents, which are compatible with the reaction conditions, will be readily apparent to one skilled in the art and alternate methods must then be used.

The reaction is preferably conducted in the presence of an inert solvent. There is no particular restriction on the nature of the solvent to be employed, provided that it has no adverse effect on the reaction or on the reagents involved and that it can dissolve the reagents, at least to some extent. Examples of suitable solvents include: aliphatic hydrocarbons, such as hexane, heptane, ligroin and petroleum ether; aromatic hydrocarbons, such as benzene, toluene and xylene; halogenated hydrocarbons, especially aromatic and aliphatic hydrocarbons, such as methylene chloride, chloroform, carbon tetrachloride, dichloroethane, chlorobenzene and the dichlorobenzenes; esters, such as ethyl formate, ethyl acetate, propyl acetate, butyl acetate and diethyl carbonate; ethers, such as diethyl ether, diisopropyl ether, tetrahydrofuran, dioxane, dimethoxyethane and diethylene glycol dimethyl ether; ketones, such as acetone, methyl ethyl ketone, methyl isobutyl ketone, isophorone and cyclohexanone; nitro compounds, which may be nitroalkanes or nitroaranes, such as nitroethane and nitrobenzene; nitriles, such as acetonitrile and isobutyronitrile; amides, which may be fatty acid amides, such as formamide, dimethylformamide, dimethylacetamide and hexamethylphosphoric triamide; and sulphoxides, such as dimethyl sulphoxide and sulpholane.

The reaction can take place over a wide range of temperatures, and the precise reaction temperature is not critical to the invention. In general, we find it convenient to carry out the reaction at a temperature of from −50° C. to 200° C.

The desired products of Formula I provided herein can be prepared as shown in Scheme 1. An appropriate nitro-halo pyridine 1 may be treated with a monoprotected diamine (such as 2) under base or metal-mediated coupling conditions to afford the coupled pyridine product 3. Subsequent deprotection of the primary amine will provide amine 4. A second nitro-halo benzamide 5 may be reacted with amine 4 under base or metal-mediated coupling conditions to afford a bis-nitro dimeric benzamide 6. Double reduction of the nitro groups via appropriate conditions can provide the diamine 7. Coupling of diamine 7 with two molecules of heteroaryl isothiocyanate (8, Y═NCO) will provide the desired product I. Alternatively, the reaction of 7 with cyanogen bromide provides the aminobenzimidazole bis amine 9. Peptide coupling between bis amine 9 and two molecules of heteroaryl acid 10 generates the desired product I.

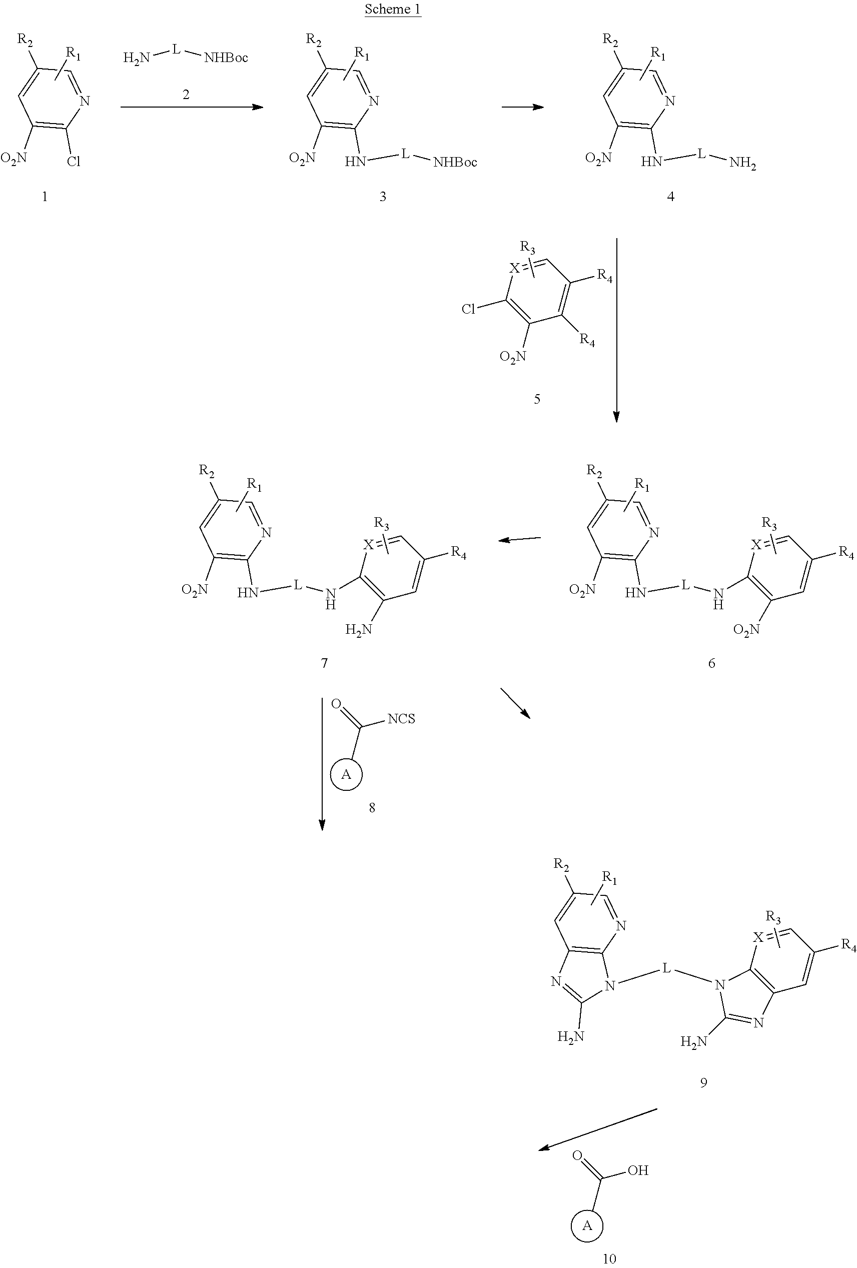

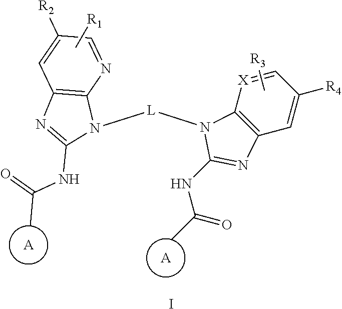

Alternatively, the desired products of Formula I provided herein can be prepared as shown in Scheme 2. An appropriate nitro-halo pyridine 1 may be treated with a monoprotected diamine (such as 2) under base or metal-mediated coupling conditions to afford the coupled pyridine product 3. The reduction of 3 under general reduction condition, followed by the reaction with cyanogen bromide will produce 11. The coupling of 11 with a heteroaryl acid, subsequent deprotection of the primary amine, will provide amine 12. Alternatively, the reduction product from 3 can react with heteroaryl isothiocyanate 8. The deprotection of the generated product will produce amine 12.

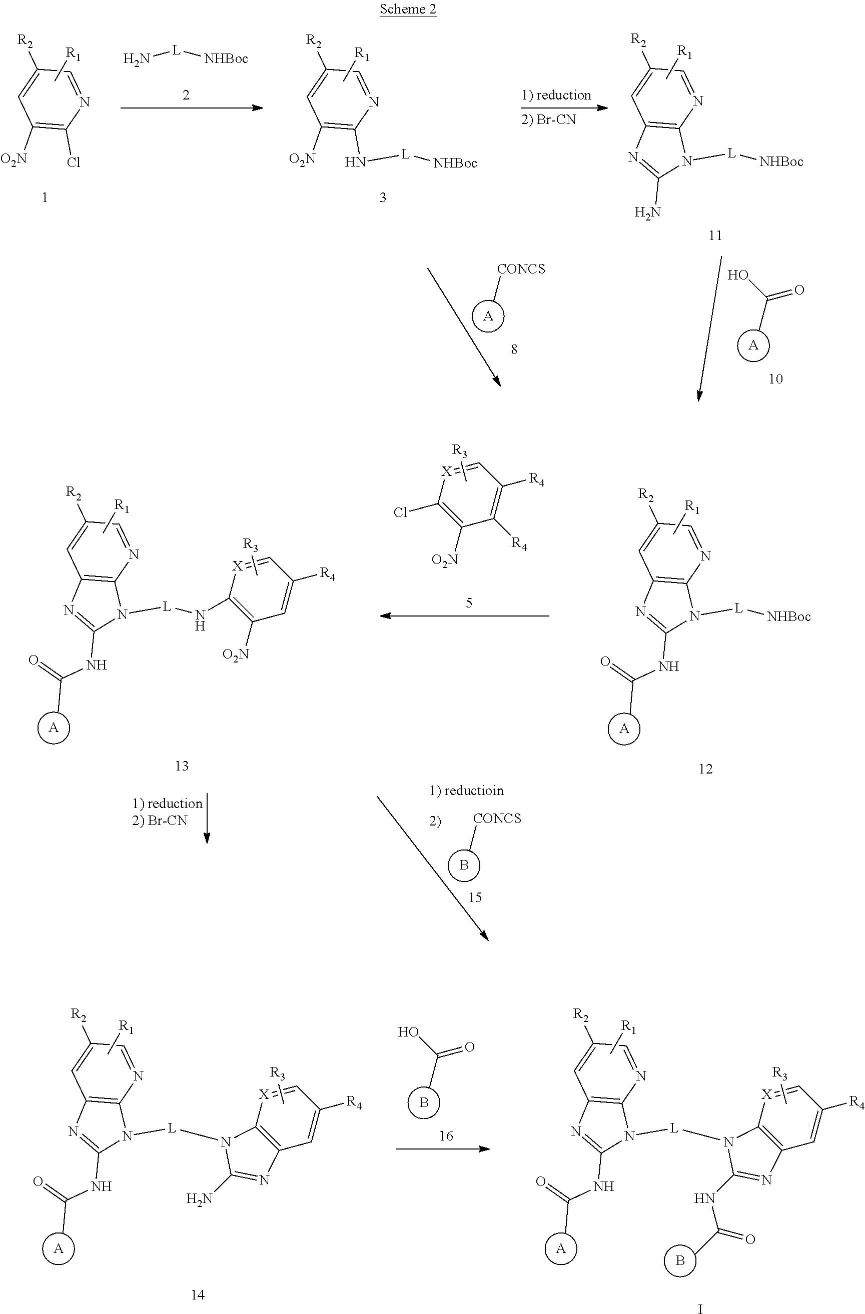

A second nitro-halo benzamide 5 may be reacted with amine 12 under base or metal-mediated coupling conditions to afford a coupling product 13. The reduction of 13 under general reduction condition, followed by the reaction with cyanogen bromide will produce the primary amine 14. Coupling of amine 14 with a heteroaryl acid 16 with peptide coupling condition will provide the desired product I. Alternatively, the reaction of the reduced product of 12 with heteroaryl isothiocyanate 15 provides product I.

The products of Formula I can also be produced as shown in Scheme 3. The initial step in the synthesis, as depicted, utilizes 2-chloro-3-nitropyridine of formula 1 as an initial starting material. The 2-chloro-3-nitropyridine 1 undergoes reaction with amine nucleophiles of formula 2, in the presence of base as a proton scavenger, at temperatures from room temperature to 200° C., to give aminonitropyridines of formula 3. The next step of the reaction is the reduction of the nitro group of formula 2 to the amine to give the diaminopyridine compound 17. This step can be carried out through palladium or nickel reduction in the presence of a hydrogen source or through stoichiometric metal reductions, such as iron and zinc in the presence of mild acid. Then the reaction of the resulted diamine 17 with oxalic ester 18 (wherein Y is represented by chloride, alkoxy, succinimide, etc.) can generate the aminopyridine aminooxoacetate compounds to provide ester 19. The hydrolysis of 19 under acid or basic condition will give heteroaryl acid 20, from which the peptide coupling reaction with amine 21 will afford amide 22.

Alternatively, formula 22 can be prepared by condensation of the diaminopyridines 17 and acid 25, which can be generated from ester oxalamides 24. And formula 24 can be generated from amines 21 of displacement on a half ester of oxalyl chloride 23, under thermal conditions, with reaction rates being increased under basic conditions. Alternatively, the reaction of the reduced product of 28 with formula 18 provides esters 30, which can result in the formation of acids 31 under acidic or basic condition. The peptide coupling of 31 and 32 affords the desired products I.

In the final step of Scheme 3, the conversion of the compounds of formula 22 to amine 26 can be accomplished under general acidic condition with high yield. With the primary amines 26 in hand, a second nitro-halo aryl 5 may be reacted with amine 26 under base or metal-mediated coupling conditions to afford a coupling product 27. The reduction of 27 under general reduction condition can form amine 28. Coupling of amine 28 with a heteroaryl acid 29, obtained with the similar procedure as that of formula 25, under the peptide coupling condition will provide the desired product I.

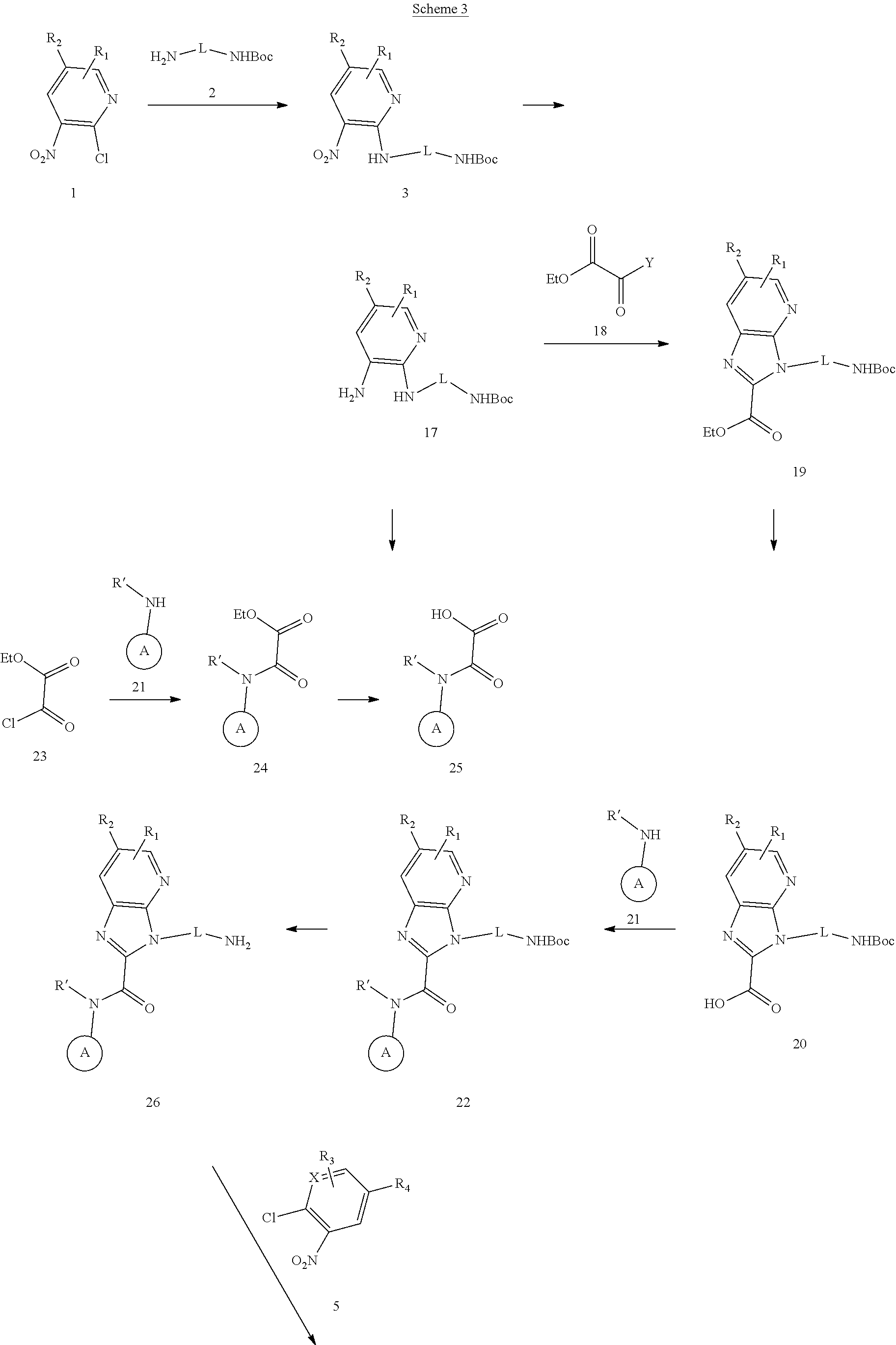

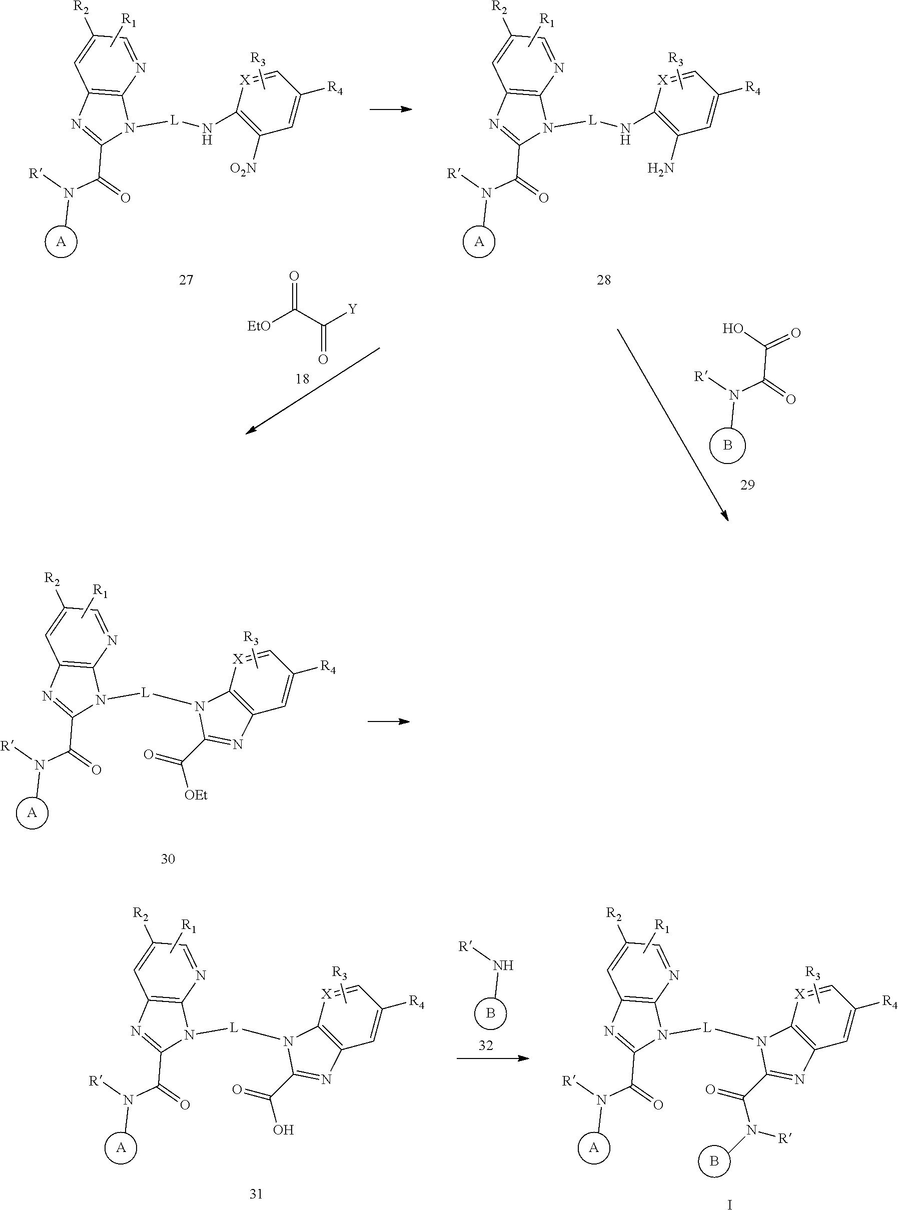

Alternatively the reaction of the resulted diamine 28 with oxalic ester 18 (wherein Y is represented by chloride, alkoxy, succinimide, etc.) can generate the aminopyridine aminooxoacetate compounds 30. The hydrolysis of 30 under acid or basic condition can give heteroaryl acid 31, from which the peptide coupling reaction with amine 32 produces the formula I.

Scheme 4 below illustrates another alternative synthetic sequence for preparation of the compounds of formula I. 2-chloro-3-nitropyridine 1 undergoes reaction with amine nucleophiles of formula 2, in the presence of base as a proton scavenger, at temperatures from room temperature to 200° C., to give aminonitropyridines of formula 3. The deprotection of 3 under acidic condition to provide amines 33, which can react with a second nitro-halo aryl (5) under base or metal-mediated coupling conditions can afford a coupling product 34.

The reduction of 34 under general reduction condition can form amines 35. This step can be carried through palladium or nickel reduction in the presence of a hydrogen source or through stoichiometric metal reductions, such as iron and zinc in the presence of mild acid.

In the next transformation, the reaction of the resulted diamine 35 with oxalic ester 18 (wherein Y is represented by chloride, alkoxy, succinimide, etc.) can generate the aminopyridine aminooxoacetate compounds 36. The hydrolysis of 36 under acid or basic condition will give heteroaryl acid 37, from which the peptide coupling reaction with amine 21 produces the formula I.

Alternatively, formula I can be prepared by condensation of the diaminopyridines 35 and acid 25, with peptide coupling condition, to provide the desired product (I).

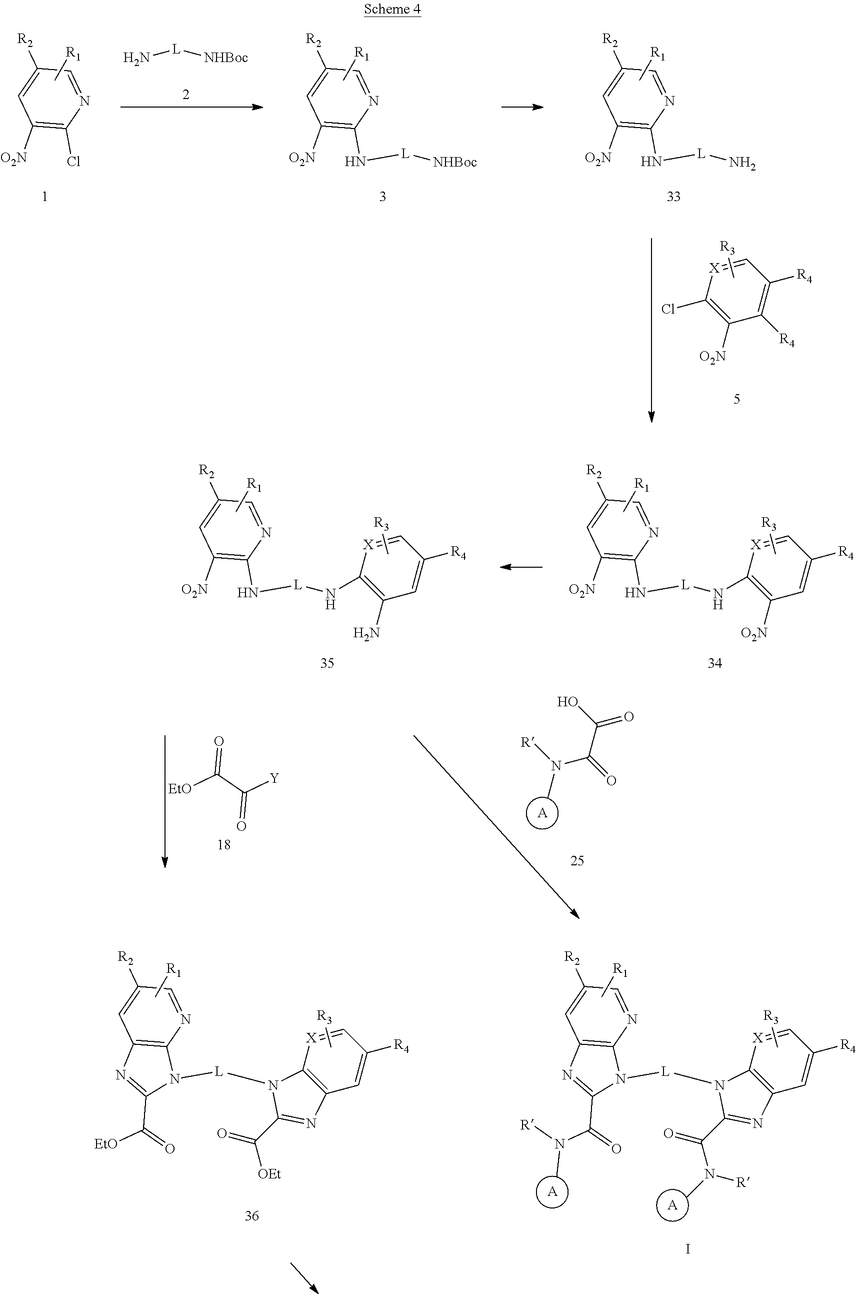

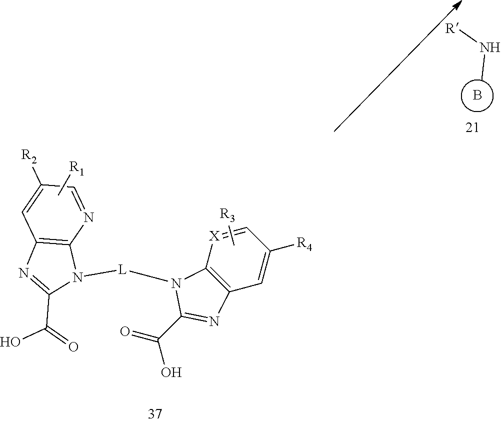

Scheme 5 below illustrates an alternative synthetic sequence for the preparation of compounds of formula I.

The reduction of formula 13 in the general reduction condition affords diamines 38. Coupling of amine 38 with a heteroaryl acid 32, under peptide coupling condition can provide the desired product I.

Alternatively the reaction of the resulted diamine 38 with oxalic ester 18 (wherein Y is represented by chloride, alkoxy, succinimide, etc.) will generate the aminopyridine aminooxoacetate compounds 39. The hydrolysis of 39 under acid or basic condition can give heteroaryl acid 40, from which the peptide coupling reaction with amine 29 produces the formula I.

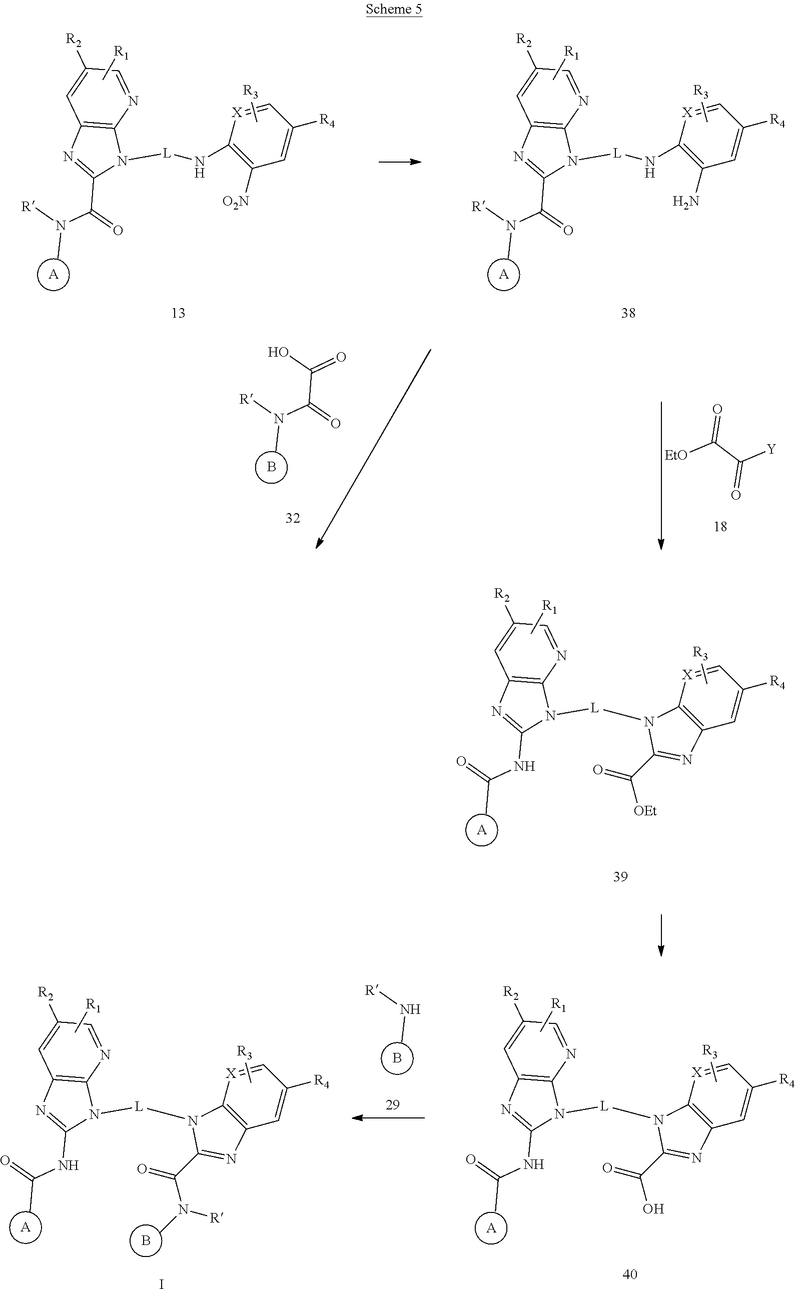

Scheme 6 demonstrcated the preparation of diamides of formula I. Coupling of diamine 28 with heteroaryl isothiocyanate 15 can provide the desired formula I. Alternatively, reaction of diamine 28 with cyanogen bromide provides the aminobenzimidazole 41. Peptide coupling between amine 40 and heteroaryl acid 16 generates the desired product (I).

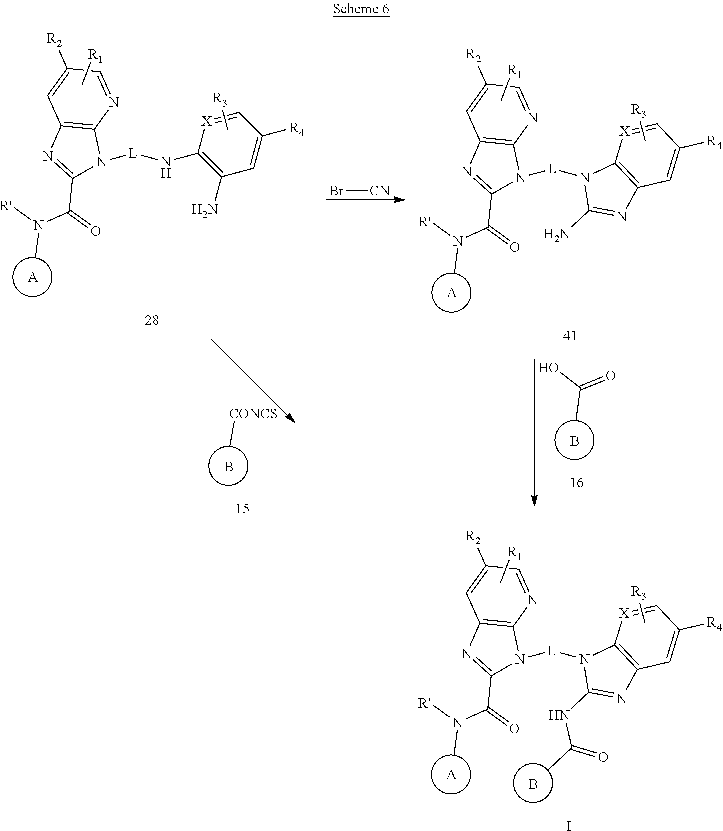

The further modification of formula I is showed in Scheme 7. Treatment of compound I with alkyl halide, or other alkylation reagents, in the presence of base can provide formula Ia.

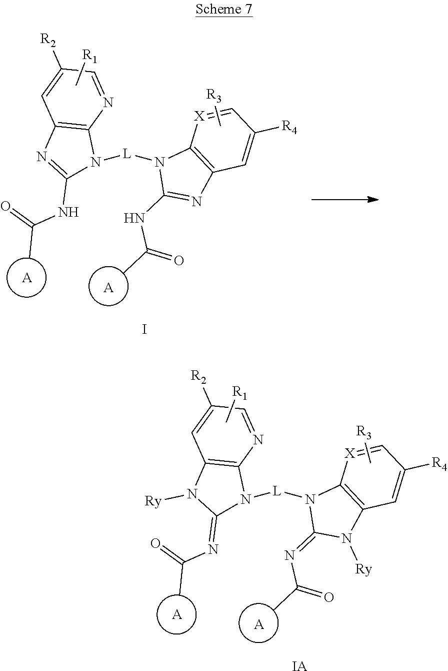

### SPECIFIC EXAMPLES

The following examples are provided to further illustrate the present invention but, of course, should not be construed as in any way limiting its scope.

All experiments were performed under anhydrous conditions (i.e. dry solvents) in an atmosphere of argon, except where stated, using oven-dried apparatus and employing standard techniques in handling air-sensitive materials. Aqueous solutions of sodium bicarbonate (NaHCO₃) and sodium chloride (brine) were saturated.

The invention will now be further described with reference to the following illustrative examples in which, unless stated otherwise:
(a) temperatures are given in degrees Celsius (° C.); operations are carried out at room temperature or ambient temperature, that is, in a range of 18-25° C.;
(b) organic solutions were dried over anhydrous sodium sulfate; evaporation of organic solvent was carried out using a rotary evaporator under reduced pressure (4.5-30 mmHg) with a bath temperature of up to 60° C.;
(c) chromatography means flash chromatography on silica gel; thin layer chromatography (TLC) was carried out on Merck Kiesel gel 60 F254 plates with visualization by ultraviolet and/or anisaldehyde, potassium permanganate or phosphomolybdic acid dips.
(d) in general, the course of reactions was followed by TLC or liquid chromatography/mass spectroscopy (LC/MS) and reaction times are given for illustration only;
(e) final products have satisfactory proton nuclear magnetic resonance (NMR) spectra and/or mass spectra data;
(f) yields are given for illustration only and are not necessarily those which can be obtained by diligent process development; preparations were repeated if more material was required;
(g) when given, NMR data is in the form of delta values for major diagnostic protons, given in parts per million (ppm) relative to tetramethylsilane (TMS) as an internal NMR spectra: 1H Nuclear magnetic resonance spectra were recorded at 400 MHz. Data are presented as follows: chemical shift, multiplicity (s=singlet, d=doublet, t=triplet, q=quartet, qn=quintet, dd=doublet of doublets, m=multiplet, bs=broad singlet), coupling constant (J/Hz) and integration. Coupling constants were taken and calculated directly from the spectra and are uncorrected.
(i) chemical symbols have their usual meanings;
(j) solvent ratio was given in volume: volume (v/v) terms. And the following abbreviations have been used: DMF—dimethylformamide; EtOAc—ethyl acetate; EtOH-ethanol; THF—tetrahydrofuran; MeOH—methanol; and DCM—dichloromethane.
(k) Low resolution mass spectra: Electrospray (ES+) ionization was used. The protonated parent ion (M+H) or parent sodium ion (M+Na) or fragment of highest mass is quoted. Analytical gradient consisted of 10% ACN in water ramping up to 100% ACN over 5 minutes unless otherwise stated.
(l) Purification of the compounds were carried out using one or more of the following methods: 1) flash chromatography on regular silica gel; 2) flash chromatography on silica gel using Isco Combiflash@ separation system: RediSep normal phase flash column, flow rate, 30-40 ml/min;

Preparation of Starting Materials:

The starting materials for the Examples contained herein are either commercially available or are readily prepared by standard methods from known materials. For examples the following reactions are illustrations but not limitations of the preparation of some of the starting materials and examples used herein.

### Intermediate 1

Preparation of tert-butyl (E)-(4-aminobut-2-en-1-yl)carbamate

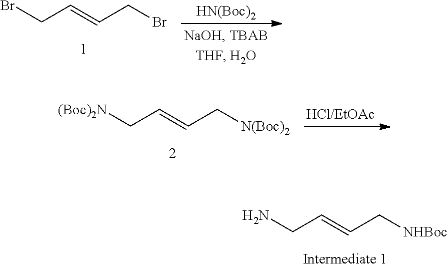

Step 1 di-tert-butyl (E)-(4-(bis(tert-butoxycarbonyl)amino)but-2-en-1-yl)iminodicarbonate To a solution of (E)-1,4-dibromobut-2-ene (300 g, 1.40 mol, 1.00 eq), tert-butyl N-tert-butoxycarbonylcarbamate (609 g, 2.81 mol, 2.00 eq) and TBAB (22.6 g, 70.1 mmol, 0.05 eq) in THF (1.80 L) was added a solution of NaOH (561 g, 14.0 mol, 10.0 eq) in H₂O (2.40 L) below 30° C. and the reaction mixture was stirred at 50° C. for 16 hrs to afford a clear solution. TLC (Petroleum ether/EtOAc=10/1, R_{f}=0.23, KMnO₄) showed one main spot with more polarity. The reaction was clean according to TLC. The reaction mixture was diluted with H₂O (400 mL) and extracted with EtOAc (1000 mL×3). The combined organic layers were washed with brine (1600 mL), dried over anhydrous Na₂SO₄, filtered and concentrated under reduced pressure to provide di-tert-butyl (E)-(4-(bis(tert-butoxycarbonyl)amino)but-2-en-1-yl)iminodicarbonate (602 g, 1.24 mol, 88.2% yield) as white solid, which was used directly in the next step without further purification.

¹HNMR (400 MHz, CDCl₃): δ (ppm) 5.67 (dt, J=2.88, 1.69 Hz, 2H), 4.04-4.17 (m, 4H), 1.44-1.54 (m, 36H).

### Step 2 tert-butyl (E)-(4-aminobut-2-en-1-yl)carbamate

To a solution of di-tert-butyl (E)-(4-(bis(tert-butoxycarbonyl)amino)but-2-en-1-yl)iminodicarbonate (300 g, 617 mmol, 1.00 eq) in EtOAc (3.00 L) was added HCl/EtOAc (4 M, 1.54 L, 10.0 eq) at 5-10° C. and the reaction mixture was stirred at 10° C. for 2 hrs to afford a white suspension. TLC (DCM/MeOH=10/1, R_{f}=0.25, KMnO₄) showed reactant was consumed completely. The reaction mixture was filtered and the filter cake was washed with EtOAc (200 mL×2). The organic phase was concentrated. The filter cake was dissolved in H₂O (800 mL). To the water phase was added solid NaOH to bring pH≈13 and extracted with DCM/MeOH (400/40 mL×3). The combined organic layer was washed with brine (300 mL), dried over anhydrous Na₂SO₄, filtered and concentrated under reduced pressure. NMR showed tert-butyl (E)-(4-aminobut-2-en-1-yl)carbamate (75.0 g, 403 mmol, 32.6% yield) was obtained as light yellow liquid.

¹HNMR (400 MHz, DMSO-d₆): δ (ppm) 6.87-7.01 (m, 1H), 5.54-5.65 (m, 1H), 5.41-5.54 (m, 1H), 3.52 (br t, J=4.94 Hz, 2H), 11 (br d, J=5.25 Hz, 2H), 1.38 (s, 9H)

Intermediate 2

Preparation of (E)-3-(4-aminobut-2-en-1-yl)-2-(1-ethyl-3-methyl-1H-pyrazole-5-carboxamido)-3H-imidazo[4,5-b]pyridine-6-carboxamide

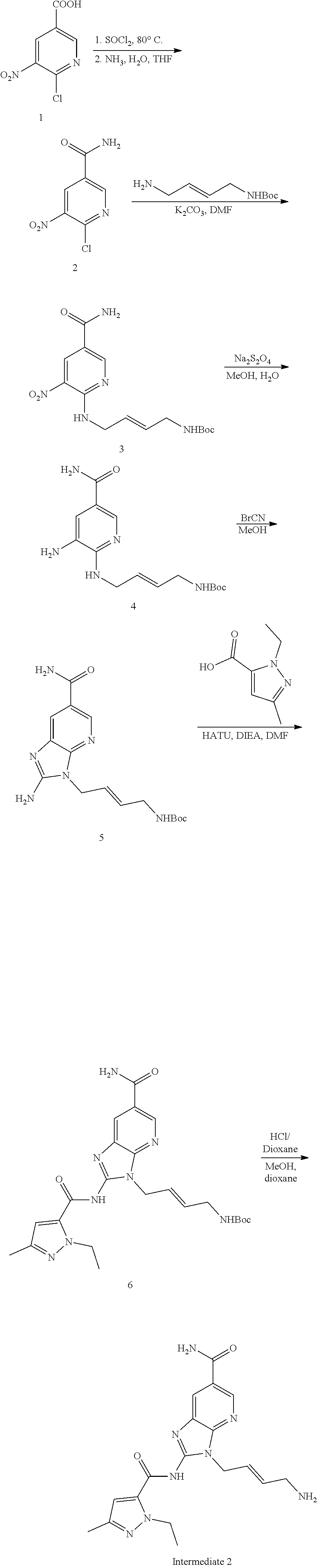

### Step 1 6-chloro-5-nitronicotinamide

To a solution of 6-chloro-5-nitronicotinic acid (125 g, 679 mmol, 1.00 eq) in SOCl₂ (748 g, 6.29 mol, 456 mL, 9.26 eq) was added drops of DMF (2 mL) and the reaction mixture was stirred at 90° C. for 16 hrs under N₂. The reaction mixture was concentrated under reduced pressure to afford acyl chloride. To NH₃·H₂O (2.38 kg, 13.6 mol, 2.62 L, 20.0% purity, 20.0 eq) was added acyl chloride in THF (750 mL) at −10˜0° C. and the reaction mixture was stirred at 0° C. for 1 hr to afford a yellow suspension. TLC (DCM/MeOH=10/1, R_{f}=0.65, UV) showed one main spot with lower polarity was detected. The reaction was clean according to TLC. The reaction mixture was filtered to give 6-chloro-5-nitronicotinamide (205 g, 956 mmol, 70.4% yield, 94.0% purity) as yellow solid. The crude product was used directly in the next step without further purification.

¹HNMR (400 MHz, MeOD): δ (ppm) 9.09 (d, J=2.13 Hz, 1H), 8.84 (d, J=2.25 Hz, 1H). LCMS: m/z 210.0 (M+1).

### Step 2 tert-butyl (E)-(4-((5-carbamoyl-3-nitropyridin-2-yl)amino)but-2-en-1-yl)carbamat

To a solution of 6-chloro-5-nitronicotinamide (100 g, 496 mmol, 1.00 eq) and tert-butyl (E)-(4-aminobut-2-en-1-yl)carbamate (Intermediate 1, example 1, 92.4 g, 496 mmol, 1.00 eq) in DMF (1.50 L) was added K₂CO₃ (103 g, 744 mmol, 1.50 eq) and the reaction mixture was stirred at 25° C. for 16 hrs under N₂ to afford a yellow solution. LCMS showed one main peak with desired MS peak was found. The reaction mixture was poured into H₂O (4000 mL) and filtered and dried to provide tert-butyl (E)-(4-((5-carbamoyl-3-nitropyridin-2-yl)amino)but-2-en-1-yl)carbamate (350 g, 837 mmol, 84.3% yield, 84.0% purity) as yellow solid. The crude product was used directly in the next step without further purification.

¹HNMR (400 MHz, DMSO-d₆): δ (ppm) 8.92 (d, J=2.00 Hz, 1H), 8.81-8.89 (m, 2H), 7.47 (br s, 1H), 6.95 (br s, 1H), 5.53-5.73 (m, 2H), 4.22 (br s, 2H), 3.53 (br s, 2H), 1.36 (s, 9H). LCMS: m/z 352.3 (M+1).

Step 3 tert-butyl (E)-(4-((3-amino-5-carbamoylpyridin-2-yl)amino)but-2-en-1-yl)carbamate To a solution of tert-butyl (E)-(4-((5-carbamoyl-3-nitropyridin-2-yl)amino)but-2-en-1-yl)carbamate (170 g, 483 mmol, 1.00 eq) in MeOH (1.70 L) and H₂O (850 mL) was added NH₃·H₂O (170 g, 968 mmol, 186 mL, 20.0% purity, 2.00 eq) followed by disodium;BLAH (295 g, 1.69 mol, 369 mL, 3.50 eq) and the reaction mixture was stirred at 25° C. for 2 hrs to afford a yellow suspension. LCMS showed major desired MS peak. The reaction mixture was diluted with H₂O (1.00 L), concentrated under reduced pressure, to the water phase was added 4N NaOH to bring pH≅10 and extracted with CHCl₃/i-PrOH (1200/400 mL×5). The combined organic layers were washed with sat. aq. NaHCO₃ (400 mL), dried over anhydrous Na₂SO₄, filtered and concentrated under reduced pressure to provide tert-butyl (E)-(4-((3-amino-5-carbamoylpyridin-2-yl)amino)but-2-en-1-yl)carbamate (210 g, 627 mmol, 64.8% yield, 96.0% purity) as brown foam, which was used directly in the next step without further purification.

¹HNMR (400 MHz, DMSO-d₆): δ (ppm) 7.96 (d, J=1.88 Hz, 1H), 7.42-7.65 (m, 1H), 7.12 (d, J=2.00 Hz, 1H), 6.77-7.01 (m, 2H), 6.18 (br t, J=5.25 Hz, 1H), 5.62-5.72 (m, 1H), 5.49-5.62 (m, 1H), 4.72-4.91 (m, 2H), 3.92-4.08 (m, 2H), 3.54 (br s, 2H), 1.37 (s, 9H). LCMS: m/z 322.2 (M+1).

### Step 4 tert-butyl (E)-(4-(2-amino-6-carbamoyl-3H-imidazo[4,5-b]pyridin-3-yl)but-2-en-1-yl)carbamate

To a solution of tert-butyl (E)-(4-((3-amino-5-carbamoylpyridin-2-yl)amino)but-2-en-1-yl)carbamate (75.0 g, 233 mmol, 1.00 eq) in MeOH (3.00 L) was added BrCN (37.1 g, 350 mmol, 25.6 mL, 1.50 eq) and the reaction mixture was stirred at 25° C. for 16 hrs under N₂. LCMS showed desired MS peak was found. The reaction mixture was concentrated under reduced pressure. The crude product was used directly in the next step without purification. tert-butyl (E)-(4-(2-amino-6-carbamoyl-3H-imidazo[4,5-b]pyridin-3-yl)but-2-en-1-yl)carbamate (80 g, 208 mmol, 88.9% yield, 89.9% purity) was obtained as black brown foam.

¹HNMR (400 MHz, CDCl₃): δ (ppm) 8.56-8.75 (m, 2H), 8.03-8.22 (m, 1H), 7.76-7.99 (m, 1H), 7.53 (br s, 1H), 7.28-7.39 (m, 1H), 6.88-7.04 (m, 1H), 5.65 (br d, J=2.75 Hz, 2H), 4.64-4.86 (m, 1H), 3.97-4.18 (m, 1H), 3.49-3.63 (m, 2H), 1.23-1.45 (m, 9H). LCMS: m/z 347.3 (M+1).

### Step 5 tert-butyl (E)-(4-(6-carbamoyl-2-(1-ethyl-3-methyl-1H-pyrazole-5-carboxamido)-3H-imidazo[4,5-b]pyridin-3-yl)but-2-en-1-yl)carbamate

To a solution of tert-butyl (E)-(4-(2-amino-6-carbamoyl-3H-imidazo[4,5-b]pyridin-3-yl)but-2-en-1-yl)carbamate (80.0 g, 231 mmol, 1.00 eq), 2-ethyl-5-methyl-pyrazole-3-carboxylic acid (35.6 g, 231 mmol, 1.00 eq) and DIPEA (149 g, 1.15 mol, 201 mL, 5.00 eq) in DMF (1.40 L) was added HATU (96.6 g, 254 mmol, 1.10 eq) and the reaction mixture was stirred at 25° C. for 16 hrs under N₂ to afford a black brown solution. LCMS showed desired MS peak was found. The reaction mixture was concentrated under reduced pressure, diluted with CHCl₃/i-PrOH (3/1 L), and washed with sat. aq. NaHCO₃ (900 mL), dried over anhydrous Na₂SO₄, filtered and concentrated under reduced pressure. The residue was purified by flash silica gel chromatography (ISCO®; 660 g×2 SepaFlash® Silica Flash Column, Eluent of 5˜10% MeOH/DCM gradient @ 300 mL/min) (DCM/MeOH=10/1, R_{f}=0.34, UV) to afford tert-butyl (E)-(4-(6-carbamoyl-2-(1-ethyl-3-methyl-1H-pyrazole-5-carboxamido)-3H-imidazo[4,5-b]pyridin-3-yl)but-2-en-1-yl)carbamate (22.0 g, 41.4 mmol, 17.9% yield, 90.9% purity) as light yellow solid.

¹HNMR (400 MHz, DMSO-d₆): δ (ppm) 12.90 (br s, 1H), 8.71-8.81 (m, 1H), 8.37 (br d, J=1.63 Hz, 1H), 8.09-8.22 (m, 2H), 7.47-7.61 (m, 1H), 6.82-7.00 (m, 1H), 6.70 (s, 1H), 5.53-5.81 (m, 2H), 4.79 (br d, J=4.63 Hz, 2H), 4.52-4.69 (m, 2H), 3.55-3.73 (m, 3H), 3.43-3.55 (m, 2H), 3.15 (q, J=6.96 Hz, 3H), 2.10-2.26 (m, 3H), 1.07-1.46 (m, 37H). LCMS: m/z 483.2 (M+1).

### Step 6 (E)-3-(4-aminobut-2-en-1-yl)-2-(1-ethyl-3-methyl-1H-pyrazole-5-carboxamido)-3H-imidazo[4,5-b]pyridine-6-carboxamide

A solution of tert-butyl (E)-(4-(6-carbamoyl-2-(1-ethyl-3-methyl-1H-pyrazole-5-carboxamido)-3H-imidazo[4,5-b]pyridin-3-yl)but-2-en-1-yl)carbamate (34.0 g, 70.5 mmol, 1.00 eq) in HCl/dioxane (4 M, 176 mL, 10.0 eq), dioxane (340 mL) and MeOH (340 mL) was stirred at 25° C. for 16 hrs to afford a light yellow slurry. LCMS showed the major desired MS peak. The filtrate was filtered and washed with DCM (60 mL×2) to provide (E)-3-(4-aminobut-2-en-1-yl)-2-(1-ethyl-3-methyl-1H-pyrazole-5-carboxamido)-3H-imidazo[4,5-b]pyridine-6-carboxamide (22.0 g, 55.8 mmol, 79.2% yield, 97.0% purity) as white solid, which was used directly in the next step without further purification.

¹HNMR (400 MHz, DMSO-d₆): δ (ppm) 8.79 (d, J=Hz, 1H), 8.24 (br s, 1H), 8.16 (d, J=Hz, 1H), 8.06 (s, 3H), 7.55 (s, 1H), 6.73 (s, 1H), 6.06 (dt, J=, 5.57 Hz, 1H), 5.60-5.76 (m, 1H), 4.84 (br d, J=Hz, 2H), 4.51-4.66 (m, 2H), 3.41 (br t, J=Hz, 2H), 2.19 (s, 3H), 1.35 (t, J=Hz, 3H). LCMS: m/z 383.2 (M+1).

Intermediate 3

Preparation of (E)-6-((4-aminobut-2-en-1-yl)amino)-5-nitronicotinamide

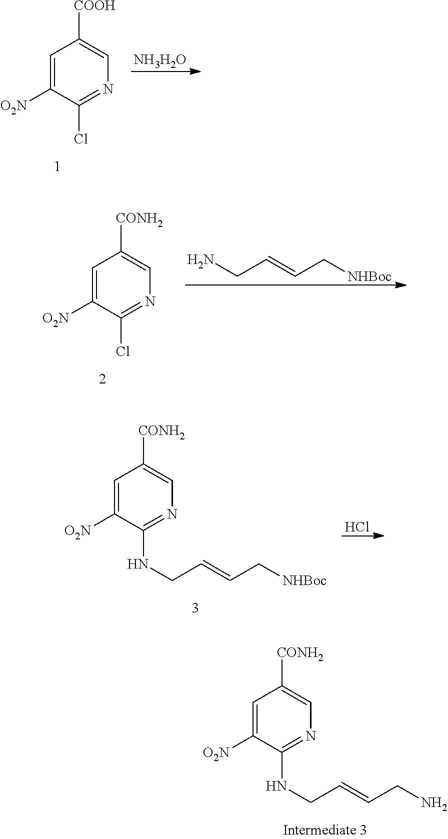

### Step 1 6-chloro-5-nitronicotinamide

6-chloro-5-nitronicotinic acid (25 g, 0.12 mol) was added to sulfurous dichloride (SOCl₂, 20 mL) and reflux 5 h. The oil-like substance was obtained after removing sulfurous dichloride (SOCl₂) under reducing pressure. The residue was dissolved in tetrahydrofuran (20 mL) under stirring in water bath, and poured into 50 mL ammonia water, and then adding 50 mL distilled water. After reaction for 1 hour, a large number of yellow solids were precipitated, which was filtered and dried to provide 6-chloro-5-nitronicotinamide as a yellow solid (9.1 g, 97%).

¹H NMR (400 MHz, DMSO-d₆): δ (ppm): 9.11 (d, J=2.0 Hz 1H), 8.92 (d, J=2.0 Hz 1H), 8.44 (s, 1H), 7.96 (s, 1H). LCMS: m/z 202 (M+1).

### Step 2: tert-butyl (E)-(4-((5-carbamoyl-3-nitropyridin-2-yl)amino)but-2-en-1-yl) carbamate

To a suspension of 6-chloro-5-nitronicotinamide (9.1 g, 45.1 mmol) in EtOH (25 mL) was added (E)-tert-butyl (4-aminobut-2-en-1-yl)carbamate (10.1 g, 54.1 mmol) and DIEA (30.4 g, 150.8 mmol). The reaction was stirred at 120° C. in a sealed tube overnight and allowed to cool to room temperature. The resulting orange solid was collected by filtration and washed with EtOH to afford tert-butyl (E)-(4-((5-carbamoyl-3-nitropyridin-2-yl)amino)but-2-en-1-yl)carbamate as a yellow solid (14.3 g, 91%).

¹H NMR (400 MHz, DMSO-d₆): δ (ppm): 8.91 (d, J=4.0, 1H), 8.88 (d, J=4.0, 1H), 8.85 (m, 1H), 8.90 (s, 1H), 7.46 (s, 1H), 6.95 (s, 1H), 5.62 (m 2H), 4.21 (t, J=5.2, 2H), 3.51 (m, 2H), 1.35 (s, 9H). LCMS: m/z 352.3 (M+1).

### Step 3: (E)-6-((4-aminobut-2-en-1-yl)amino)-5-nitronicotinamide

To a suspension of tert-butyl (E)-(4-((5-carbamoyl-3-nitropyridin-2-yl)amino)but-2-en-1-yl)carbamate (14.3 g, 40.6 mmol) in methanol (100 mL) was added slowly 4 M HCl in dioxane (200 mL, 800 mmol). The reaction mixture was stirred at room temperature for overnight. The resulting solid was isolated by filtration, washed with water 3 times (3×20 mL), and dried under high vacuum to provide (E)-6-((4-aminobut-2-en-1-yl)amino)-5-nitronicotinamide (6.3 g, 62%).

¹H NMR (400 MHz, DMSO-d₆): δ (ppm): 8.92 (d, J=2.0, 1H), 8.80 (d, J=2.0, 1H), 8.31 (m, 1H), 8.18 (s, 1H), 7.46 (s, 1H), 5.70 (m 2H), 4.27 (m, 2H), 3.32 (m, 2H), 3.11 (m, 2H). LCMS: m/z 252.1 (M+1).

Intermediate 4

Preparation of 1-ethyl-3-methyl-1H-pyrazole-5-carbonyl isothiocyanate

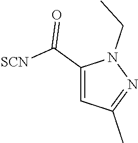

### Step 1: (1-ethyl-3-methyl-1H-pyrazole-5-carbonyl chloride

To a 1 L round bottom flask was added 1-ethyl-3-methyl-1H-pyrazole-5-carboxylic acid (6.0 g, 38.9 mmol) and DCM (100 mL). To this heterogeneous mixture was added DMF (0.1 mL, 1.291 mmol) followed by the slow addition of oxalyl chloride (5.9 g, 46.7 mmol). During the addition, bubbling was noticed. After stirring for 1 hr at room temperature, the volatiles were removed under vacuum and the crude was co-evaporated twice with dichloromethane (50 mL each). The crude (1-ethyl-3-methyl-1H-pyrazole-5-carbonyl chloride (6.7 g) was used directly in the next reaction without further purification.

Step 2 1-ethyl-3-methyl-1H-pyrazole-5-carbonyl isothiocyanate

To a dry 500 mL round bottom flask was added KSCN (4.9 g, 50.6 mmol) and acetone (200 ml). This clear homogenous solution was cooled to 0° C. After 5 min stirring at 0° C., 1-ethyl-3-methyl-1H-pyrazole-5-carbonyl chloride (6.7 g, 38.9 mmol) was added as a solution in acetone (15 mL). Once the addition was complete, the reaction was allowed to stir at 0° C. After 1 min, additional KSCN was added (2 g) and the reaction was stirred for an additional 20 min. At this time, hexanes (200 mL) were added to the reaction mixture and the crude heterogeneous solution was concentrated in vacuo to one third of the volume. The process of hexanes addition and concentration was repeated twice (50 mL of hexanes each). After the last concentration, hexanes (50 mL) were added and the solid was removed by filtration, rinsed with hexanes (50 mL). The resulted clear light-yellow filtrate was concentrated and purified by silica gel chromatography (0-20% EtOAc/petroleum) to provide 1-ethyl-3-methyl-1H-pyrazole-5-carbonyl isothiocyanate (2.8 g, 36% yield) as a clear liquid. 1H NMR (400 MHz, CDCl₃) δ ppm 6.73 (s, 1H), 4.49 (q, J=7.2 Hz, 2H), 2.29 (s, 3H), 1.40 (t, J=7.2 Hz, 3H). LCMS: m/z 196.1 (M+1). The acylisothiocyanate product degrades over time, and so a ˜0.4 M 1,4-dioxane solution was prepared and frozen to avoid/slow decomposition. This solution was thawed and used directly in subsequent reactions.

### Example 1

### (E)-3-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide

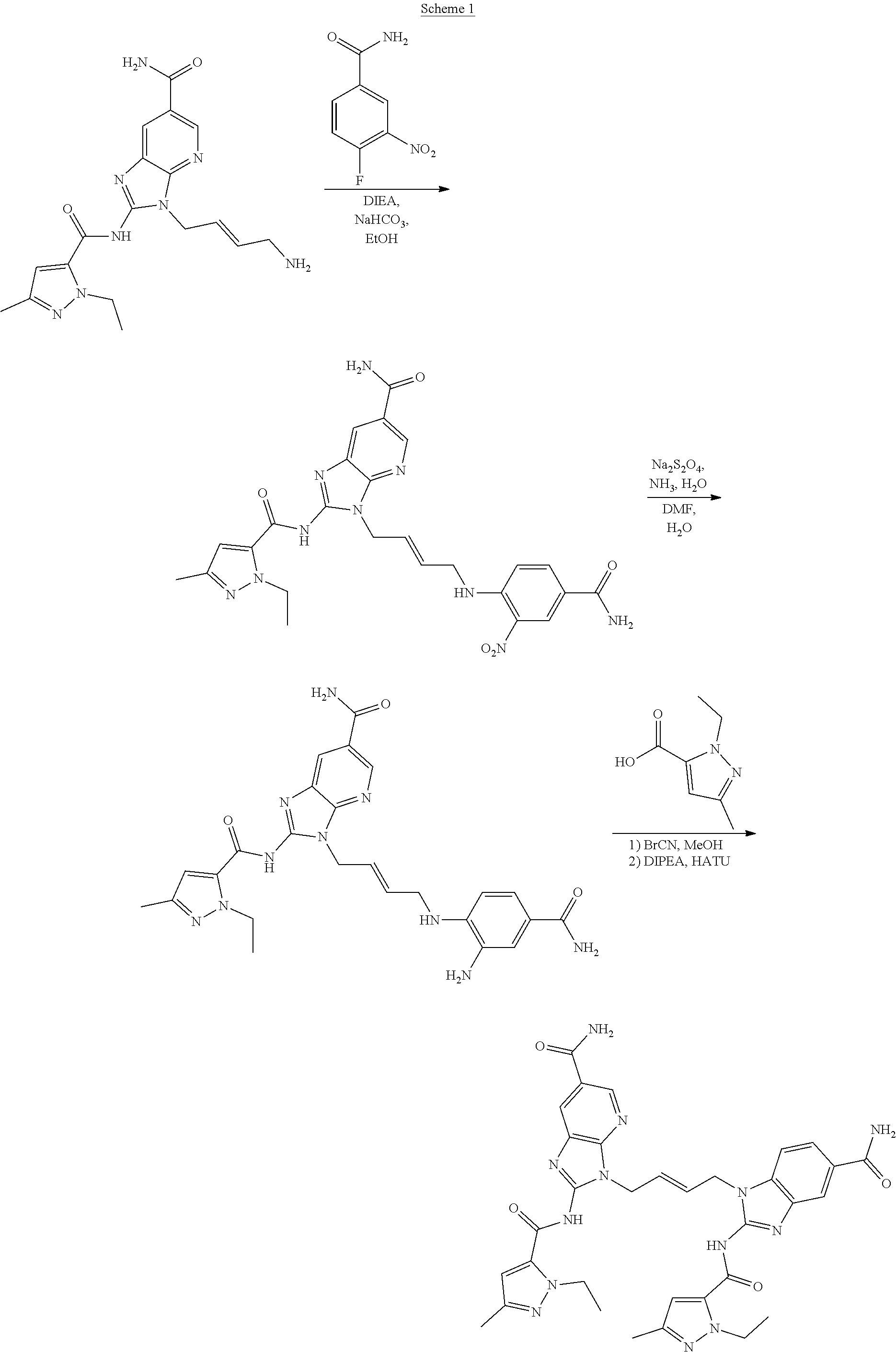 [SMILES: CCn1nc(C)cc1C(=O)Nc1nc2cc(C(N)=O)cnc2n1C/C=C/CN ; NC(=O)c1ccc(F)c([N+](=O)[O-])c1 ; CCn1nc(C)cc1C(=O)Nc1nc2cc(C(N)=O)cnc2n1C/C=C/CNc1ccc(C(N)=O)cc1[N+](=O)[O-] ; CCn1nc(C)cc1C(=O)Nc1nc2cc(C(N)=O)cnc2n1C/C=C/CNc1ccc(C(N)=O)cc1N ; CCn1nc(C)cc1C(=O)O ; CCn1nc(C)cc1C(=O)Nc1nc2cc(C(N)=O)ccc2n1C/C=C/Cn1c(NC(=O)c2cc(C)nn2CC)nc2cc(C(N)=O)cnc21]

### Step 1: (E)-3-(4-((4-carbamoyl-2-nitrophenyl)amino)but-2-en-1-yl)-2-(1-ethyl-3-methyl-1H-pyrazole-5-carboxamido)-3H-imidazo[4,5-b]pyridine-6-carboxamide

To a solution of (E)-3-(4-aminobut-2-en-1-yl)-2-(1-ethyl-3-methyl-1H-pyrazole-5-carboxamido)-3H-imidazo[4,5-b]pyridine-6-carboxamide (Intermediate 2, 6.00 g, 14.342 mmol, 1.00 eq, HCl), 4-fluoro-3-nitro-benzamide (2.64 g, 14.3 mmol, 1.00 eq), DIPEA (7.40 g, 57.3 mmol, 9.98 mL, 4.00 eq) and NaHCO₃ (4.81 g, 57.30 mmol, 2.23 mL, 4 eq) in EtOH (60 mL) was stirred at 110° C. for 16 hrs under N₂ to afford a yellow suspension. LCMS showed one main peak with desired MS peak was found. The reaction mixture was diluted with H₂O (60 mL) and filtered to provide (E)-3-(4-((4-carbamoyl-2-nitrophenyl)amino)but-2-en-1-yl)-2-(1-ethyl-3-methyl-1H-pyrazole-5-carboxamido)-3H-imidazo[4,5-b]pyridine-6-carboxamide (5.0 g, 9.14 mmol, 63.80% yield, 99.9% purity) was obtained as yellow solid, which was used directly in the next step without further purification.

¹HNMR (400 MHz, DMSO-d₆): δ (ppm) 12.83 (br s, 1H), 8.72 (d, J=1.50 Hz, 1H), 8.62 (d, J=2.00 Hz, 1H), 8.49 (t, J=5.94 Hz, 1H), 8.13 (br s, 2H), 7.86-8.01 (m, 2H), 7.51 (br s, 1H), 7.25 (br s, 1H), 6.95 (d, J=9.13 Hz, 1H), 6.59 (s, 1H), 5.86-5.96 (m, 1H), 5.72-5.83 (m, 1H), 4.81 (br d, J=4.88 Hz, 2H), 4.55 (q, J=7.05 Hz, 2H), 4.05 (br s, 2H), 2.15 (s, 3H), 1.31 (t, J=7.07 Hz, 3H). LCMS: m/z 547.2 (M+1).

### Step 2 (E)-3-(4-((2-amino-4-carbamoylphenyl)amino)but-2-en-1-yl)-2-(1-ethyl-3-methyl-1H-pyrazole-5-carboxamido)-3H-imidazo[4,5-b]pyridine-6-carboxamide

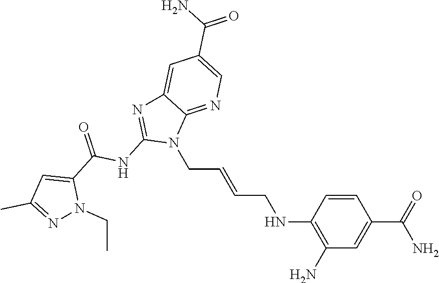 [SMILES: CCn1nc(C)cc1C(=O)Nc1nc2cc(C(N)=O)cnc2n1C/C=C/CNc1ccc(C(N)=O)cc1N]

To a solution of (E)-3-(4-((4-carbamoyl-2-nitrophenyl)amino)but-2-en-1-yl)-2-(1-ethyl-3-methyl-1H-pyrazole-5-carboxamido)-3H-imidazo[4,5-b]pyridine-6-carboxamide (5.00 g, 9.15 mmol, 1.00 eq) in DMF (50 mL) and H₂O (25 mL) was added NH₃·H₂O (12.8 g, 91.4 mmol, 14.0 mL, 25% purity, 10.0 eq) followed by disodium;BLAH (4.78 g, 27.4 mmol, 5.97 mL, 3.00 eq) and the reaction mixture was stirred at 25° C. for 1 hr to afford a yellow suspension. LCMS showed desired MS peak was found. The reaction mixture was diluted with H₂O (500 mL) and lyophilized. The residue was diluted with DMF (400 mL) and filtered. The filtrate was concentrated in vacuum. (E)-3-(4-((2-amino-4-carbamoylphenyl)amino)but-2-en-1-yl)-2-(1-ethyl-3-methyl-1H-pyrazole-5-carboxamido)-3H-imidazo[4,5-b]pyridine-6-carboxamide (4.5 g, crude) was obtained as light yellow solid, which was used directly in the next step without purification. LCMS: m/z 517.3 (M+1).

### Step 3 (E)-3-(4-(2-amino-5-carbamoyl-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide

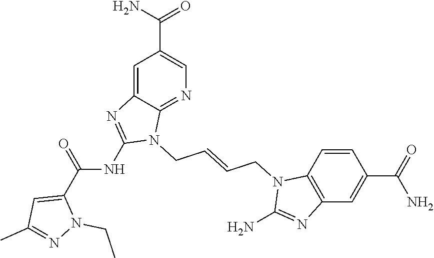 [SMILES: CCn1nc(C)cc1C(=O)Nc1nc2cc(C(N)=O)cnc2n1C/C=C/Cn1c(N)nc2cc(C(N)=O)ccc21]

To a solution of (E)-3-(4-((2-amino-4-carbamoylphenyl)amino)but-2-en-1-yl)-2-(1-ethyl-3-methyl-1H-pyrazole-5-carboxamido)-3H-imidazo[4,5-b]pyridine-6-carboxamide (4.5 g, 8.71 mmol, 1 eq) in DMF (45 mL) and MeOH (90 mL) was added BrCN (2.77 g, 26.13 mmol, 1.92 mL, 3 eq) and the reaction mixture was stirred at 50° C. for 2 hr under N₂ to afford a yellow suspension. LCMS showed the desired MS peak. The reaction mixture was concentrated under reduced pressure. The residue was triturated with EtOAc/i-PrOH (30/10 mL) to afford (E)-3-(4-(2-amino-5-carbamoyl-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide (3.20 g, 5.06 mmol, 58.1% yield, 98.5% purity, HBr) yellow solid. LCMS: m/z 542.4 (M+1).

### Step 4 (E)-3-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide

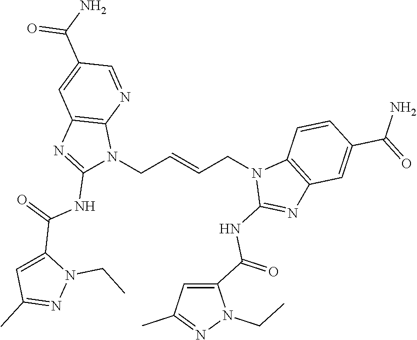 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cnc21]

To a solution of (E)-3-(4-(2-amino-5-carbamoyl-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide (1.00 g, 1.61 mmol, 1.00 eq, HBr), 1-ethyl-3-methyl-1H-pyrazole-5-carboxylic acid (297 mg, 1.93 mmol, 1.20 eq) and DIEA (1.04 g, 8.03 mmol, 1.40 mL, 5.00 eq) in DMF (15.0 mL) was added HATU (794 mg, 2.09 mmol, 1.30 eq) and the reaction mixture was stirred at 50° C. for 16 hrs under N₂ to afford a yellow solution. LCMS showed the desired MS peak. The reaction mixture was filtered. The filter cake was washed with cold DMF (5 mL×2) and lyophilized. (E)-3-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide (1.40 g) as light yellow solid.

¹HNMR (400 MHz, DMSO-d₆): δ (ppm) 12.64-13.11 (m, 2H), 8.71 (s, 1H), 8.12 (s, 2H), 7.85-8.00 (m, 2H) 7.71 (d, J=8.13 Hz, 1H), 7.26-7.58 (m, 3H), 6.55 (s, 2H), 5.79-6.07 (m, 2H), 4.89-4.76 (m, 4H), 4.38-4.59 (m, 4H), 2.12 (s, 6H), 1.06-1.35 (m, 6H). LCMS: m/z 678.5 (M+1). HPLC: 93% purity.

### Example 2

### (E)-3-(4-(5-carbamoyl-2-(1-ethyl-3-methyl-1H-pyrazole-5-carboxamido)-7-methoxy-1H-benzo[d]imidazol-1-yl)but-2-en-1-yl)-2-(l-ethyl-3-methyl-1H-pyrazole-5-carboxamido)-3H-imidazo[4,5-b]pyridine-6-carboxamide

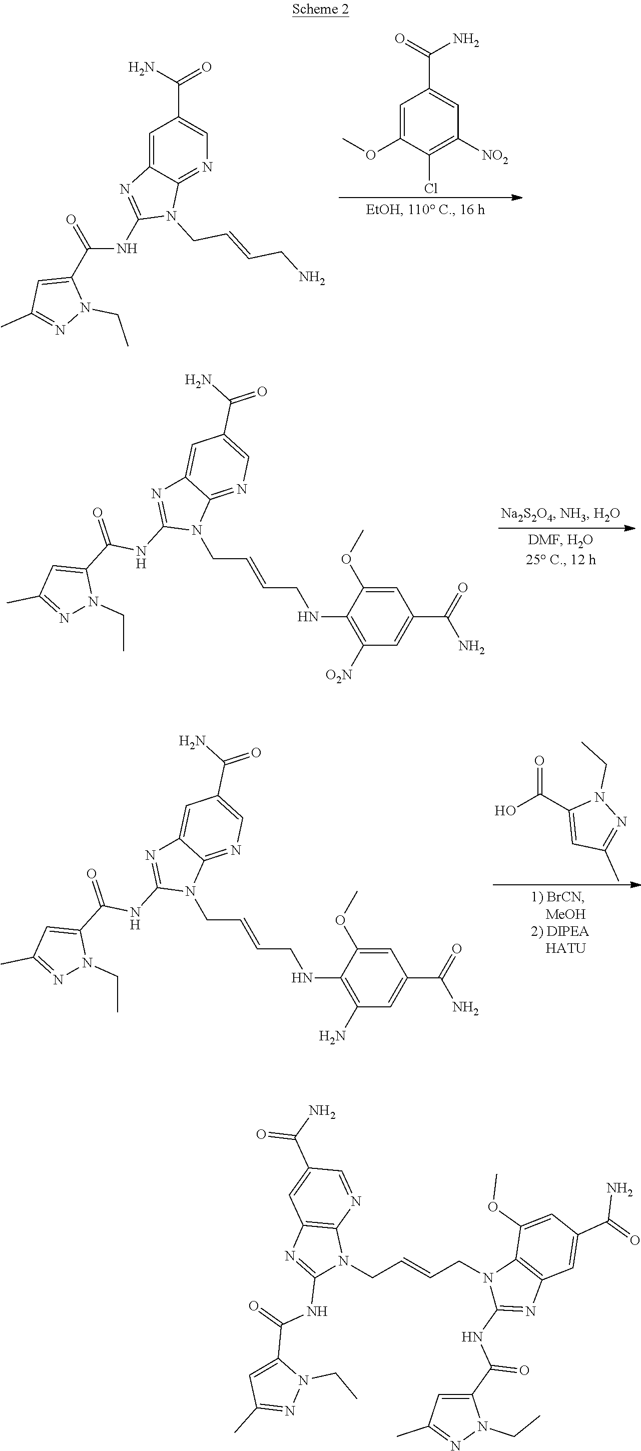 [SMILES: CCn1nc(C)cc1C(=O)Nc1nc2cc(C(N)=O)cnc2n1C/C=C/CN ; COc1cc(C(N)=O)cc([N+](=O)[O-])c1Cl ; CCn1nc(C)cc1C(=O)Nc1nc2cc(C(N)=O)cnc2n1C/C=C/CNc1c(OC)cc(C(N)=O)cc1[N+](=O)[O-] ; CCn1nc(C)cc1C(=O)Nc1nc2cc(C(N)=O)cnc2n1C/C=C/CNc1c(N)cc(C(N)=O)cc1OC ; CCn1nc(C)cc1C(=O)O ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OC)c21]

### Step 1 (E)-3-(4-((4-carbamoyl-2-methoxy-6-nitrophenyl)amino)but-2-en-1-yl)-2-(1-ethyl-3-methyl-1H-pyrazole-5-carboxamido)-3H-imidazo[4,5-b]pyridine-6-carboxamide

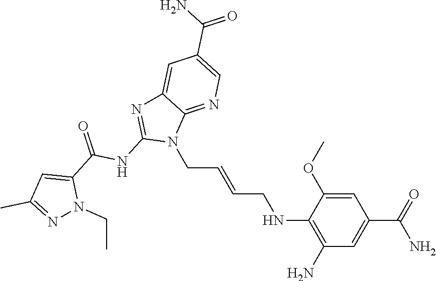 [SMILES: CCn1nc(C)cc1C(=O)Nc1nc2cc(C(N)=O)cnc2n1C/C=C/CNc1c(N)cc(C(N)=O)cc1OC]

A solution of (E)-3-(4-aminobut-2-en-1-yl)-2-(1-ethyl-3-methyl-1H-pyrazole-5-carboxamido)-3H-imidazo[4,5-b]pyridine-6-carboxamide (Intermediate 2, 6.00 g, 14.3 mmol, 1.00 eq, HCl), 4-chloro-3-methoxy-5-nitro-benzamide (3.30 g, 14.3 mmol, 1.00 eq) and DIEA (7.41 g, 57.3 mmol, 9.98 mL, 4.00 eq) in EtOH (50.0 mL) was stirred at 110° C. for 16 hrs under N₂ to afford a yellow suspension. LCMS showed the desired product MS peak. The reaction mixture was diluted with 1120 (50 mL) and filtered to give (E)-3-(4-((4-carbamoyl-2-methoxy-6-nitrophenyl)amino)but-2-en-1-yl)-2-(1-ethyl-3-methyl-1H-pyrazole-5-carboxamido)-3H-imidazo[4,5-b]pyridine-6-carboxamide (3.50 g, 5.63 mmol, 39.3% yield, 92.8% purity) as yellow solid, which was used directly in the next step without further purification.

¹HNMR (400 MHz, DMSO-d₆): δ (ppm) 8.72 (s, 1H), 8.31 (s, 1H), 8.14 (s, 2H), 8.10 (d, J=1.63 Hz, 1H), 8.06 (d, J=1.63 Hz, 1H), 7.97 (s, 1H), 7.89 (d, J=1.63 Hz, 1H), 7.79 (s, 1H), 7.71 (t, J=6.25 Hz, 1H), 7.53 (s, 1H), 7.48 (d, J=1.50 Hz, 1H), 7.31 (s, 1H), 6.64 (s, 1H), 5.73-5.82 (m, 2H), 4.77 (d, J=3.75 Hz, 2H), 4.57 (q, J=6.80 Hz, 2H), 4.07 (d, J=5.00 Hz, 2H), 4.03 (s, 2H), 3.76 (s, 3H), 2.17 (s, 3H), 1.33 (t, J=7.07 Hz, 3H). LCMS: m/z 577.2 (M+1).

### Step 2 (E)-3-(4-((2-amino-4-carbamoyl-6-methoxyphenyl)amino)but-2-en-1-yl)-2-(1-ethyl-3-methyl-1H-pyrazole-5-carboxamido)-3H-imidazo[4,5-b]pyridine-6-carboxamide

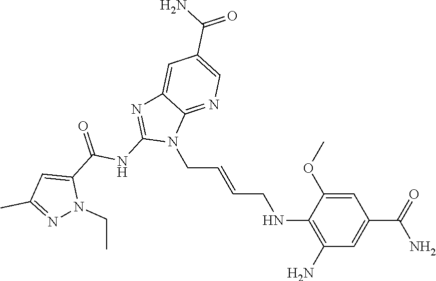 [SMILES: CCn1nc(C)cc1C(=O)Nc1nc2cc(C(N)=O)cnc2n1C/C=C/CNc1c(N)cc(C(N)=O)cc1OC]

To a solution of (E)-3-(4-((4-carbamoyl-2-methoxy-6-nitrophenyl)amino)but-2-en-1-yl)-2-(1-ethyl-3-methyl-1H-pyrazole-5-carboxamido)-3H-imidazo[4,5-b]pyridine-6-carboxamide (3.50 g, 6.07 mmol, 1.00 eq) in DMF (30 mL) and H₂O (15 mL) was added NH₃·H₂O (8.51 g, 60.7 mmol, 9.35 mL, 25.0% purity, 10.0 eq) followed by disodium;BLAH (3.17 g, 18.2 mmol, 3.96 mL, 3.00 eq) and the reaction mxiture was stirred at 25° C. for 1 hr to a yellow solution. LCMS showed desired MS peak was found. The reaction mixture was diluted with H₂O (400 mL) and lyophilized. The residue was diluted with DMF (400 mL) and filtered. The filtrate was concentrated in vacuum. The residue was purified by flash silica gel chromatography (ISCO@; 40 g SepaFlash® Silica Flash Column, Eluent of 10˜20% MeOH/DCM gradient @ 40 mL/min) (DCM/MeOH=3/1, Rf=0.24, UV) to afford (E)-3-(4-((2-amino-4-carbamoyl-6-methoxyphenyl)amino)but-2-en-1-yl)-2-(1-ethyl-3-methyl-1H-pyrazole-5-carboxamido)-3H-imidazo[4,5-b]pyridine-6-carboxamide (1.60 g, 2.78 mmol, 45.8% yield, 95.1% purity) as light yellow solid. LCMS: m/z 547.5 (M+1).

### Step 3 (E)-3-(4-(2-amino-5-carbamoyl-7-methoxy-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide

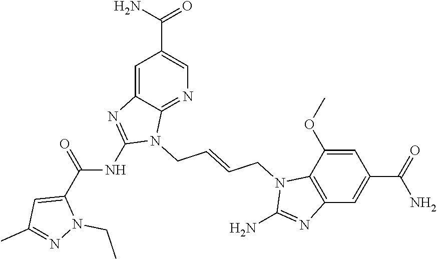 [SMILES: CCn1nc(C)cc1C(=O)Nc1nc2cc(C(N)=O)cnc2n1C/C=C/Cn1c(N)nc2cc(C(N)=O)cc(OC)c21]

To a solution of (E)-3-(4-((2-amino-4-carbamoyl-6-methoxyphenyl)amino)but-2-en-1-yl)-2-(1-ethyl-3-methyl-1H-pyrazole-5-carboxamido)-3H-imidazo[4,5-b]pyridine-6-carboxamide (1.60 g, 2.93 mmol, 1.00 eq) in DMF (15 mL) and MeOH (30 mL) was added BrCN (465.0 mg, 4.39 mmol, 322 uL, 1.50 eq) and the reaction mixture was stirred at 50° C. for 2 hr under N₂ to afford a white suspension. LCMS showed reactant was consumed completely. The reaction mixture was concentrated in vacuum. The residue was triturated with iPrOH/EtOAc (10/30 mL) for 1 hour to provide (E)-3-(4-(2-amino-5-carbamoyl-7-methoxy-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide (1.74 g, 2.62 mmol, 89.5% yield, 98.3% purity, HBr) as light yellow solid, which was used directly in the next step without further purification. LCMS: m/z 572.2 (M+1).

### Step 4 (E)-3-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide

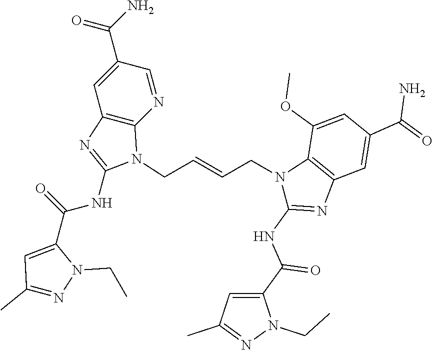 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OC)c21]

To a solution of 1-ethyl-3-methyl-1H-pyrazole-5-carboxylic acid (493 mg, 3.20 mmol, 1.20 eq) and DIEA (1.38 g, 10.6 mmol, 1.86 mL, 4.00 eq) in DMF (30 mL) was added HATU (1.32 g, 3.47 mmol, 1.30 eq) followed by (E)-3-(4-(2-amino-5-carbamoyl-7-methoxy-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide (1.74 g, 2.67 mmol, 1.00 eq, HBr) and the reaction mixture was stirred at 50° C. for 16 hrs under N₂ to afford a yellow suspension. LCMS showed desired MS peak was found. The reaction mixture was concentrated in vacuum. The residue was purified by flash silica gel chromatography (ISCO®; 40 g SepaFlash® Silica Flash Column, Eluent of 10˜20% MeOH/DCM gradient @ 60 mL/min) (DCM/MeOH=3/1, R_{f}=0.43, UV) to afford (E)-3-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide (450 mg, 605 μmol, 22.7% yield, 95.3% purity) as yellow solid.

¹HNMR (400 MHz, DMSO-d₆): δ (ppm) 12.79 (br s, 2H), 8.64-8.81 (m, 1H), 8.53 (d, J=3.63 Hz, 1H), 8.34 (d, J=8.38 Hz, 1H), 7.87-8.18 (m, 3H), 7.42-7.77 (m, 2H), 7.24-7.37 (m, 3H), 6.52 (d, J=18.26 Hz, 2H), 5.63-6.07 (m, 2H), 4.70-5.01 (m, 4H), 4.51 (d, J=6.63 Hz, 4H), 3.79 (s, 3H), 2.04-2.16 (m, 6H), 1.18-1.25 (m, 6H). LCMS: m/z 708.6 (M+1).

### Example 3

### (E)-3-(4-(5-carbamoyl-2-(1-ethyl-3-methyl-1H-pyrazole-5-carboxamido)-7-(3-hydroxypropoxy)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide

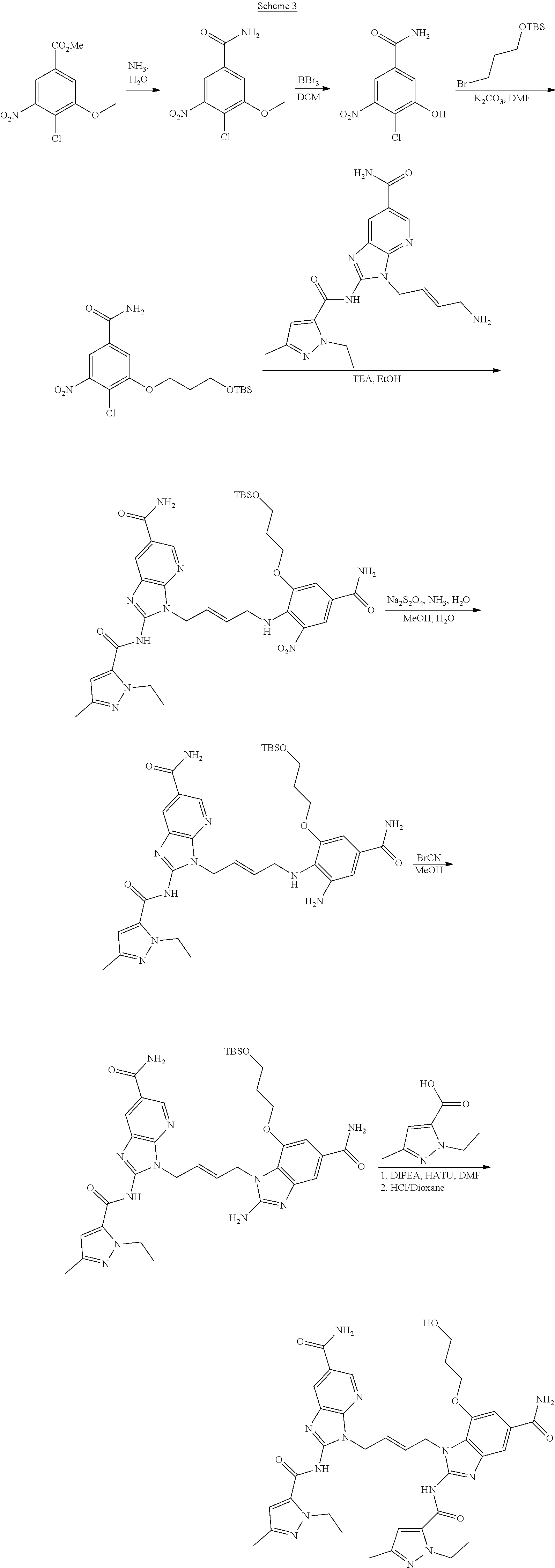

### Step 1 4-chloro-3-methoxy-5-nitrobenzamide

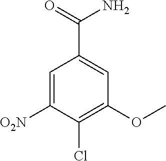

A solution of methyl 4-chloro-3-methoxy-5-nitrobenzoate (20.0 g, 81.4 mmol, 1.00 eq) in NH₃·H₂O (218 g, 1.56 mol, 240 mL, 25.0% purity, 19.1 eq) was stirred at 50° C. for 72 hrs. A yellow suspension was obtained. The mixture was cooled to room temperature and was filtered to afford 4-chloro-3-methoxy-5-nitrobenzamide (14.2 g, 54.1 mmol, 66.4% yield, 87.9% purity) as a light yellow solid, which was used directly in the next step without further purification. ¹HNMR (400 MHz, DMSO-d₆): δ (ppm) 8.29 (br s, 1H), 8.05 (d, J=1.75 Hz, 1H), 7.88 (d, J=1.75 Hz, 1H), 7.78 (br s, 1H), 4.02 (s, 3H). LCMS: m/z 231.1 (M+1).

### Step 2 4-chloro-3-hydroxy-5-nitrobenzamide

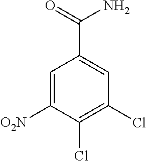 [SMILES: NC(=O)c1cc(Cl)c(Cl)c([N+](=O)[O-])c1]

To a solution of 4-chloro-3-methoxy-5-nitrobenzamide (35.0 g, 151 mmol, 1.00 eq) in DCM (600 mL) was added BBr₃ (152 g, 607 mmol, 58.5 mL, 4.00 eq) and the reaction mixture was stirred at 50° C. for 36 hrs. A yellow suspension was obtained. LCMS (EB69-6-P1B) showed Reactant 1 was consumed completely and one main peak with desired MS was detected. The reaction mixture was poured into ice water (1.50 L) and the mixture was stirred at 15° C. for 30 min, and extracted with EtOAc (1.5 L×3). The combined organic layers were washed with brine (500 mL×2), dried over anhydrous Na₂SO₄, filtered and concentrated under reduced pressure to give a residue. The crude product was triturated with petroleum ether/ethyl acetate (1:1, 130 mL) at 20° C. for 30 min to give 4-chloro-3-hydroxy-5-nitrobenzamide (23.6 g, 96.9 mmol, 63.8% yield) as a light yellow solid.

¹HNMR (400 MHz, DMSO-d₆): δ (ppm) 11.55 (br s, 1H), 8.17 (br s, 1H), 7.92 (d, J=1.75 Hz, 1H), 7.60-7.76 (m, 2H). LCMS: m/z 231.1 (M+1). LCMS: m/z 217.0 (M+1).

### Step 3 3-(3-((tert-butyldimethylsilyl)oxy)propoxy)-4-chloro-5-nitrobenzamide

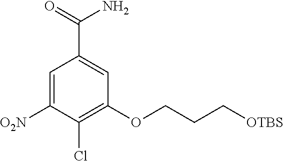 [SMILES: CC(C)(C)[Si](C)(C)OCCCOc1cc(C(N)=O)cc([N+](=O)[O-])c1Cl]

To a solution of 4-chloro-3-hydroxy-5-nitrobenzamide (13.2 g, 61.3 mmol, 1.00 eq) in DMF (130 mL) was added K₂CO₃ (16.9 g, 122 mmol, 2.00 eq) followed by (3-bromopropoxy)(tert-butyl)dimethylsilane (18.6 g, 73.6 mmol, 1.20 eq) and the reaction mixture was stirred at 100° C. under N₂ for 2 hrs. A brown suspension was obtained. The reaction mixture was poured into water (250 mL) and stirred at 20° C. for 30 min, filtered to afford the filter cake, which was triturated with petroleum ether/ethyl acetate (5:1, 120 mL) at 25° C. for 30 min to produce 3-(3-((tert-butyldimethylsilyl)oxy)propoxy)-4-chloro-5-nitrobenzamide (19.8 g, 49.5 mmol, 80.7% yield) as a light yellow solid.

¹HNMR (400 MHz, DMSO-d₆): δ (ppm) 8.28 (br s, 1H), 8.04 (d, J=1.63 Hz, 1H), 7.88 (d, J=1.63 Hz, 1H), 7.77 (br s, 1H), 4.29 (t, J=5.88 Hz, 2H), 3.79 (t, J=6.07 Hz, 2H), 1.97 (quin, J=5.94 Hz, 2H), 0.84 (s, 9H), 0.01 (s, 6H). LCMS: m/z 389.1 (M+1).

### Step 4 (E)-3-(4-((2-(3-((tert-butyldimethylsilyl)oxy)propoxy)-4-carbamoyl-6-nitrophenyl)amino)but-2-en-1-yl)-2-(1-ethyl-3-methyl-1H-pyrazole-5-carboxamido)-3H-imidazo[4,5-b]pyridine-6-carboxamide

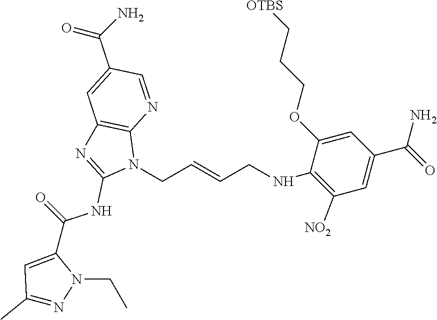 [SMILES: CCn1nc(C)cc1C(=O)Nc1nc2cc(C(N)=O)cnc2n1C/C=C/CNc1c(OCCCO[Si](C)(C)C(C)(C)C)cc(C(N)=O)cc1[N+](=O)[O-]]

To a solution of 3-(3-((tert-butyldimethylsilyl)oxy)propoxy)-4-chloro-5-nitrobenzamide (7.00 g, 18.0 mmol, 1.00 eq) in EtOH (60.0 mL) was added (E)-3-(4-aminobut-2-en-1-yl)-2-(1-ethyl-3-methyl-1H-pyrazole-5-carboxamido)-3H-imidazo[4,5-b]pyridine-6-carboxamide (Intermediate 2, Example 1, 8.29 g, 19.8 mmol, 1.10 eq, HCl) followed by TEA (7.29 g, 71.9 mmol, 10.0 mL, 4.00 eq) and the reaction mixture was stirred at 120° C. for 12 hrs. A red solution was obtained. The reaction mixture was concentrated under reduced pressure to remove solvent. The residue was purified by flash silica gel chromatography (ISCO@; 120 g SepaFlash@ Silica Flash Column, Eluent of 50.0˜90.0% Ethylacetate/Petroleum ethergradient 10% Methanol/Dichloromethane, @ 80 mL/min_Plate 1_Dichloromethane/Methanol=10:1, R_{f} of product=0.66, UV 254 nm) to produce (E)-3-(4-((2-(3-((tert-butyldimethylsilyl)oxy)propoxy)-4-carbamoyl-6-nitrophenyl)amino)but-2-en-1-yl)-2-(1-ethyl-3-methyl-1H-pyrazole-5-carboxamido)-3H-imidazo[4,5-b]pyridine-6-carboxamide (9.13 g, 8.93 mmol, 49.6% yield) as an orange solid. LCMS: m/z 735.3 (M+1).

### Step 5 (E)-3-(4-((2-amino-6-(3-((tert-butyldimethylsilyl)oxy)propoxy)-4-carbamoylphenyl)amino)but-2-en-1-yl)-2-(1-ethyl-3-methyl-1H-pyrazole-5-carboxamido)-3H-imidazo[4,5-b]pyridine-6-carboxamide

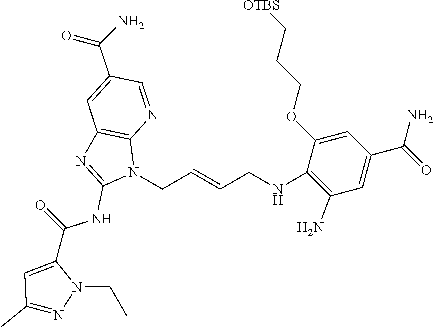 [SMILES: CCn1nc(C)cc1C(=O)Nc1nc2cc(C(N)=O)cnc2n1C/C=C/CNc1c(N)cc(C(N)=O)cc1OCCCO[Si](C)(C)C(C)(C)C]

To a solution of (E)-3-(4-((2-(3-((tert-butyldimethylsilyl)oxy)propoxy)-4-carbamoyl-6-nitrophenyl)amino)but-2-en-1-yl)-2-(1-ethyl-3-methyl-1H-pyrazole-5-carboxamido)-3H-imidazo[4,5-b]pyridine-6-carboxamide (Intermediate 2, 9.13 g, 12.4 mmol, 1.00 eq) in DMF (90.0 mL) and H₂O (45.0 mL) was added NH₃·H₂O (17.4 g, 124 mmol, 19.1 mL, 25.0% purity, 10.0 eq) followed by disodium; BLAH (6.49 g, 37.2 mmol, 8.11 mL, 3.00 eq) and then the reaction mixture was stirred at 20° C. for 2 hrs. A light yellow suspension was obtained. To the reaction mixture was added water (500 mL) and the solvent was removed by lyophilization. The residue was dissolved by CDCl₃/i-PrOH=3:1 (800 mL) filtered and concentrated under reduced pressure to give a residue. The residue was purified by flash silica gel chromatography (ISCO@; 80.0 g SepaFlash@ Silica Flash Column, Eluent of 10.0-20.0% Methanol/Dichloromethane @100 mL/min_Dichloromethane/Methanol=10:1, Rr of product=0.18, UV 254 nm) to give (E)-3-(4-((2-amino-6-(3-((tert-butyldimethylsilyl)oxy)propoxy)-4-carbamoylphenyl)amino)but-2-en-1-yl)-2-(1-ethyl-3-methyl-1H-pyrazole-5-carboxamido)-3H-imidazo[4,5-b]pyridine-6-carboxamide (1.09 g, 11.5% yield) as a yellow solid. LCMS: m/z 705.6 (M+1).

### Step 6 (E)-3-(4-(2-amino-7-(3-((tert-butyldimethylsilyl)oxy)propoxy)-5-carbamoyl-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide

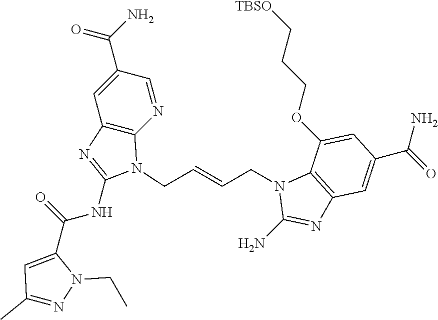 [SMILES: CCn1nc(C)cc1C(=O)Nc1nc2cc(C(N)=O)cnc2n1C/C=C/Cn1c(N)nc2cc(C(N)=O)cc(OCCCO[Si](C)(C)C(C)(C)C)c21]

To a solution of (E)-3-(4-((2-amino-6-(3-((tert-butyldimethylsilyl)oxy)propoxy)-4-carbamoylphenyl)amino)but-2-en-1-yl)-2-(1-ethyl-3-methyl-1H-pyrazole-5-carboxamido)-3H-imidazo[4,5-b]pyridine-6-carboxamide (1.09 g, 1.43 mmol, 1.00 eq) in MeOH (40.0 mL) was added BrCN (159.0 mg, 1.50 mmol, 110 uL, 1.05 eq) and the reaction mixture was stirred at 25° C. for 1 hr under N₂. A yellow suspension was obtained. The reaction mixture was concentrated under reduced pressure to remove solvent. The reaction mixture was triturated with EtOAc (50.0 mL) at 25° C. for 30 min and filtered to produce (E)-3-(4-(2-amino-7-(3-((tert-butyldimethylsilyl)oxy)propoxy)-5-carbamoyl-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide (1.05 g, 1.14 mmol, 79.4% yield) as a brown solid. LCMS: m/z 30.3 (M+1).

### Step 7 (E)-3-(4-(7-(3-((tert-butyldimethylsilyl)oxy)propoxy)-5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide

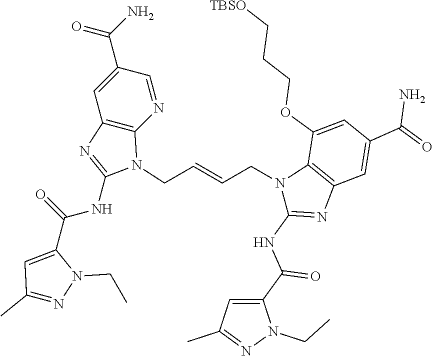 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCO[Si](C)(C)C(C)(C)C)c21]

To a mixture of (E)-3-(4-(2-amino-7-(3-((tert-butyldimethylsilyl)oxy)propoxy)-5-carbamoyl-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide and 1-ethyl-3-methyl-1H-pyrazole-5-carboxylic acid (219 mg, 1.42 mmol, 1.10 eq) in DMF (25.0 mL) was added HATU (590 mg, 1.55 mmol, 1.20 eq) followed by DIEA (836 mg, 6.47 mmol, 1.13 mL, 5.00 eq) after the reaction mixture was clear. The mixture was stirred at 50° C. for 12 hrs. A yellow suspension was obtained. LCMS showed the reaction complete and one main peak with desired MS was detected. The reaction mixture was concentrated under reduced pressure to remove DMF to afford (E)-3-(4-(7-(3-((tert-butyldimethylsilyl)oxy)propoxy)-5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide (1.16 g, crude) as a yellow oil, which was used directly in the next step without further purification. LCMS: m/z 866.4 (M+1).

### Step 8 (E)-3-(4-(5-carbamoyl-2-(1-ethyl-3-methyl-1H-pyrazole-5-carboxamido)-7-(3-hydroxypropoxy)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide

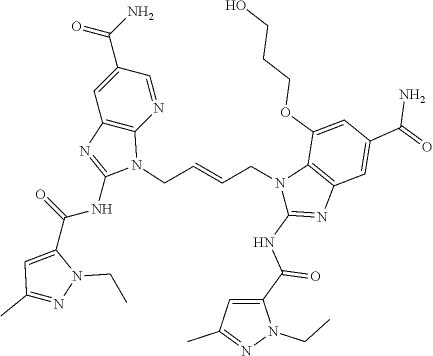 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCO)c21]

To a solution of (E)-3-(4-(7-(3-((tert-butyldimethylsilyl)oxy)propoxy)-5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide (1.16 g, 1.34 mmol, 1.00 eq) in MeOH (25.0 mL) and dioxane (25.0 mL) was added HCl/dioxane (4.00 M, 50.0 mL, 149 eq) and the reaction mixture was stirred at 25° C. for 1 hr. A yellow solution was obtained. LCMS showed the reactant was consumed completely and one main peak with the desired MS was detected. The reaction mixture was concentrated under reduced pressure to give a residue. The residue was purified by flash silica gel chromatography (ISCO@; 40.0 g SepaFlash@ Silica Flash Column, Eluent of 20.0˜40.0% Ethyl acetate/Petroleum ether gradient @ 40.0 mL/min_Plate 1, Methanol/Dichloromethane=1:10, Rr of product=0.00, UV 254 nm) to produce (E)-3-(4-(5-carbamoyl-2-(1-ethyl-3-methyl-1H-pyrazole-5-carboxamido)-7-(3-hydroxypropoxy)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide (630 mg, 0.766 mmol, 57.2% yield, 91.5% purity) as a brown solid.

¹HNMR (400 MHz, DMSO-d₆): δ (ppm) 8.71 (d, J=1.75 Hz, 1H), 8.08-8.22 (m, 2H), 7.90-8.08 (m, 1H), 7.64 (s, 1H), 7.52 (br s, 1H), 7.35 (s, 2H), 6.53 (s, 2H), 5.79-5.96 (m, 2H), 4.95 (br d, J=4.38 Hz, 2H), 4.78 (br d, J=4.63 Hz, 2H), 4.51 (dt, J=13.04, 6.68 Hz, 7H), 4.11 (br t, J=6.25 Hz, 2H), 3.46 (t, J=6.00 Hz, 2H), 2.10 (s, 6H), 1.75 (quin, J=6.07 Hz, 2H), 1.26 (t, J=7.13 Hz, 6H); LCMS: m/z 752.6 (M+1).

### Example 4

### (E)-3-(4-(5-carbamoyl-2-(1-ethyl-3-methyl-1H-pyrazole-5-carboxamido)-7-(3-morpholinopropoxy)-1H-benzo[d]imidazol-1-yl)but-2-en-1-yl)-2-(l-ethyl-3-methyl-1H-pyrazole-5-carboxamido)-3H-imidazo[4,5-b]pyridine-6-carboxamide

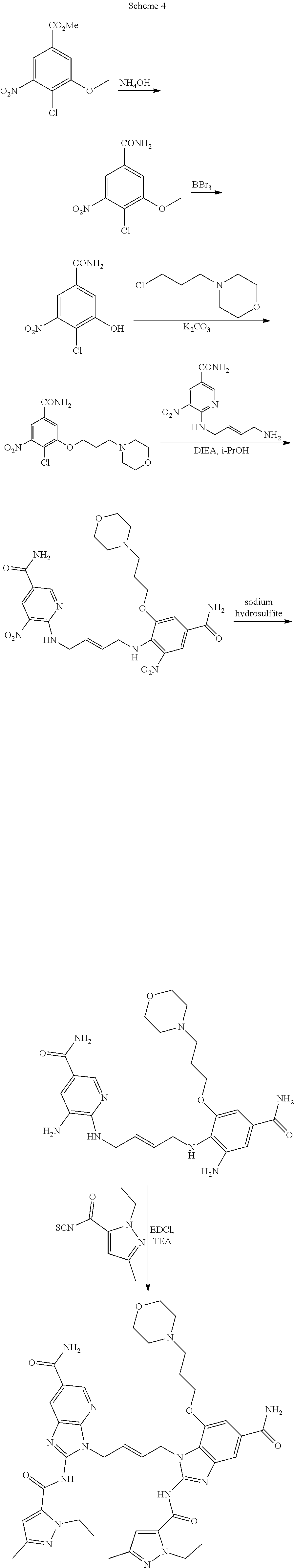

### Step 1: 4-Chloro-3-methoxy-5-nitrobenzamide

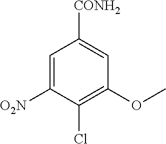

4-chloro-3-methoxy-5-nitrobenzoate (25 g, 10.1 mmol) was stirred in NH4OH (250 mL 1.9 mmol) at room temperature for 24 hrs. The reaction temperature was then increased to 50° C. for 2 hrs. An additional 50 mL (˜3.7 eq) of NH4OH was added to the vessel. After an additional 2 hrs stirring at 50° C. the reaction mixture was cooled to room temperature. The solid was filtered and rinsed with cold water. The solid was dried under house vacuum and lyophilized to give 4-chloro-3-methoxy-5-nitrobenzamide (13 g, 68% yield) as a tan solid.

¹H NMR (400 MHz, DMSO-d₆): δ (ppm): 8.31 (br. s., 1H), 8.06 (d, J=1.8 Hz, 1H), 7.88 (d, J=1.8 Hz, 1H), 7.81 (br. s., 1H), 4.02 (s, 3H). LCMS: m/z 230.9 (M+1).

### Step 2: 4-Chloro-3-hydroxy-5-nitrobenzamide

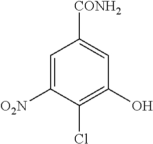 [SMILES: NC(=O)c1cc(O)c(Cl)c([N+](=O)[O-])c1]

4-Chloro-3-methoxy-5-nitrobenzamide (16 g, 69.3 mmol) was suspended in dry DCM (250 mL) and stirred at room temperature. To the reaction was added BBr3 (280 mL, 1M in DCM) dropwise. A slurry rapidly formed which was stirred overnight at room temperature under nitrogen. The reaction was poured into ice water (3 L) and stirred vigorously for 30 min. The resulting suspension was filtered and the solids dried to afford 4-chloro-3-hydroxy-5-nitrobenzamide (11.6 g, 77% yield). ¹H NMR (400 MHz, DMSO-d₆): δ (ppm): 11.53 (br. s., 1H), 8.17 (br. s., 1H), 7.92 (s, 1H), 7.72 (s, 1H), 7.66 (br. s., 1H). LC-MS. [M+H]⁺=217. LCMS: m/z 217.0 (M+1).

### Step 3 4-Chloro-3-(3-morpholinopropoxy)-5-nitrobenzamide

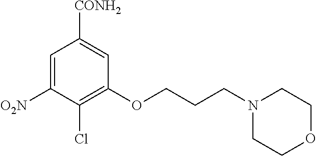 [SMILES: NC(=O)c1cc(OCCCN2CCOCC2)c(Cl)c([N+](=O)[O-])c1]

A mixture of 4-chloro-3-hydroxy-5-nitrobenzamide (11.6 g, 53.5 mmol), 4-(3-chloropropyl)morpholine (10.5 g, 64.2 mmol), K₂CO₃ (9.6 g, 69.6 mmol) in DMF (100 mL) was stirred at 70° C. overnight. Solvent was removed in vacuo to give a crude solid product that was purified by silica gel chromatography (MeOH:DCM=1:10) to give 4-chloro-3-(3-morpholinopropoxy)-5-nitrobenzamide (10.5 g, 57% yield) as a yellow solid. ¹H NMR (400 MHz, DMSO-d₆): δ (ppm): 8.30 (s, 1H), 8.05 (d, J=1.8 Hz, 1H), 7.88 (d, J=1.8 Hz, 1H), 7.80 (s, 1H), 4.28 (t, J=6.2 Hz, 2H), 3.57 (t, J=4.6 Hz, 4H), 2.41-2.47 (m, 2H), 2.37 (br. s., 4H), 1.97 (dd, J=13.94, 7.35 Hz, 2H); LCMS: m/z 344.1 (M+1).

### Step 4 (E)-6-((4-((4-carbamoyl-2-(3-morpholinopropoxy)-6-nitrophenyl)amino) but-2-en-1-yl)amino)-5-nitronicotinamide

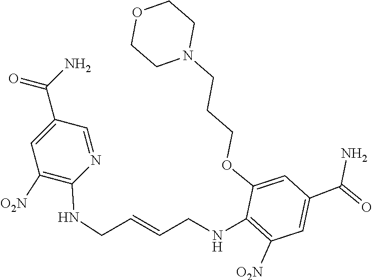 [SMILES: NC(=O)c1cnc(NC/C=C/CNc2c(OCCCN3CCOCC3)cc(C(N)=O)cc2[N+](=O)[O-])c([N+](=O)[O-])c1]

The solution of (E)-6-((4-aminobut-2-en-1-yl)amino)-5-nitronicotinamide (Intermediate 3, 220 mg, 0.87 mmol), 4-chloro-3-(3-morpholinopropoxy)-5-nitrobenzamide (200 mg, 0.58 mmol), i-PrOH (5 mL) and DIEA (1.12 g, 8.7 mmol) in a microwave vial was irradiated at 120° C. for 6 hrs. When cool, the resulting solid was isolated by filtration, rinsed with i-PrOH (2×1 mL) and dried to afford (E)-6-((4-((4-carbamoyl-2-(3-morpholinopropoxy)-6-nitrophenyl)amino)but-2-en-1-yl)amino)-5-nitronicotinamide (113 mg, 35%) as a red solid.

### Step 5 (E)-5-amino-6-((4-((2-amino-4-carbamoyl-6-(3-morpholinopropoxy) phenyl)amino)but-2-en-1-yl)amino)nicotinamide

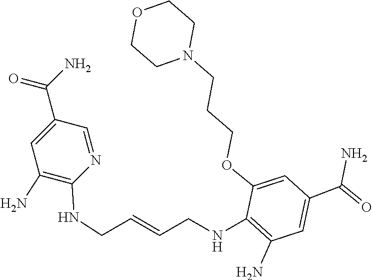 [SMILES: NC(=O)c1cnc(NC/C=C/CNc2c(N)cc(C(N)=O)cc2OCCCN2CCOCC2)c(N)c1]

To (E)-6-((4-((4-carbamoyl-2-(3-morpholinopropoxy)-6-nitrophenyl)amino)but-2-en-1-yl) amino)-5-nitronicotinamide (2.7 g, 4.8 mmol) in MeOH (40.0 mL) at room temperature was added sodium hydrosulfite (11.7 g, 67.2 mmol) in water (45 mL). After 15 min, solid sodium bicarbonate (24 g) was added. After 10 min, the reaction mixture was filtered, and the solid was rinsed with MeOH (4×20 mL). The combined filtrates were concentrated onto Celite and purified by preparative HPLCto afford (E)-5-amino-6-((4-((2-amino-4-carbamoyl-6-(3-morpholinopropoxy)phenyl)amino)but-2-en-1-yl)amino)nicotinamide (1.81 g, 3.26 mmol, 49% yield) as a dark yellow solid. ¹H NMR (400 MHz, DMSO-d₆): δ (ppm): 7.93 (s, 1H), 7.60 (m, 1H), 7.10 (s, 1H), 6.96 (br s, 1H), 6.85 (m, 2H), 6.77 (s, 1H), 6.14 (m, 1H), 5.73 (m, 2H), 4.81 (m, 2H), 4.66 (m 2H), 3.96 (m, 4H), 3.83 (m, 1H), 3.54 (m, 6H), 2.39 (t, J=7.2 Hz, 2H), 2.32 (br s, 4H), 1.84 (t, J=6.4 Hz, 2H). LCMS: m/z 499.3 (M+1).

### Step 6 (E)-3-(4-(5-carbamoyl-2-(1-ethyl-3-methyl-1H-pyrazole-5-carboxamido)-7-(3-morpholinopropoxy)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide

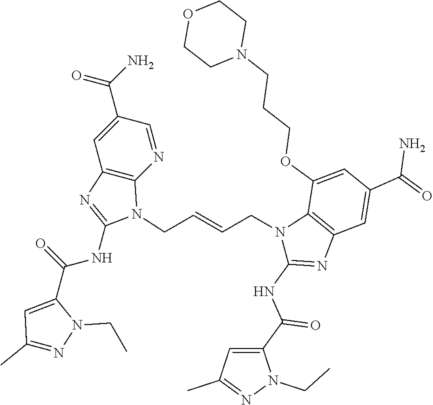 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCOCC3)c21]

To (E)-5-amino-6-((4-((2-amino-4-carbamoyl-6-(3-morpholinopropoxy)phenyl) amino)but-2-en-1-yl)amino)nicotinamide (812 mg, 1.62 mmol) in DMF (20 mL) at 0° C. was added 0.4 M 1-ethyl-3-methyl-1H-pyrazole-5-carbonyl isothiocyanate in dioxane (Intermediate 4, 6 mL, 3.89 mmol). After ˜10 min, another portion of 0.4 M 1-ethyl-3-methyl-1H-pyrazole-5-carbonyl isothiocyanate in dioxane (Intermediate 3, 2 ml, 0.48 mmol) was added, followed ˜15 min later by a final portion (2 ml, 0.48 mmol). After 35 min total reaction time, EDC (1.087 g, 5.67 mmol) was added followed by triethylamine (656 mg, 6.48 mmol). The mixture was warmed to room temperature and stirred overnight. The reaction was quenched with 3:1 water: saturated aqueous NH4Cl solution (10 mL) and extracted with 3:1 chloroform:ethanol (2×40 mL). The combined organic phases were washed with water (10 mL), dried over magnesium sulfate and concentrated. The resulting residue was purified by preparative HPLC and the desired eluents were lyophilized to give (E)-3-(4-(5-carbamoyl-2-(1-ethyl-3-methyl-1H-pyrazole-5-carboxamido)-7-(3-morpholinopropoxy)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide as white solid (445 mg, yield 45%; HPLC purity: 97.7% (254 nm)).

¹H-NMR (400 MHz, DMSO-d₆): δ (ppm): 8.70 (s, 1H), 8.14 (s, 1H), 7.66 (s, 1H), 7.33 (s, 1H), 6.51 (d, J=12.0 Hz 2H), 5.95 (m, 1H), 5.74 (m, 1H), 4.94 (d, J=4.0 Hz, 2H), 4.79 (d, J=4.8 Hz, 2H), 4.52 (m, 4H), 4.13 (t, J=11.2 Hz 2H), 3.98 (m, 2H), 3.67 (s, 2H), 3.38 (t, J=12.4 Hz, 2H), 3.24 (m, 2H), 3.05 (m, 2H), 2.09 (s, 6H), 2.06 (m, 2H), 1.26 (t, J=14.0 Hz, 6H). LCMS: m/z 821.4 (M+1), 819.3 (M−1).

### Example 5

### 3-(4-(5-carbamoyl-2-(1-ethyl-3-methyl-1H-pyrazole-5-carboxamido)-7-(3-morpholinopropoxy)-1H-benzo[d]imidazol-1-yl)butyl)-2-(1-ethyl-3-methyl-1H-pyrazole-5-carboxamido)-3H-imidazo[4,5-b]pyridine-6-carboxamide

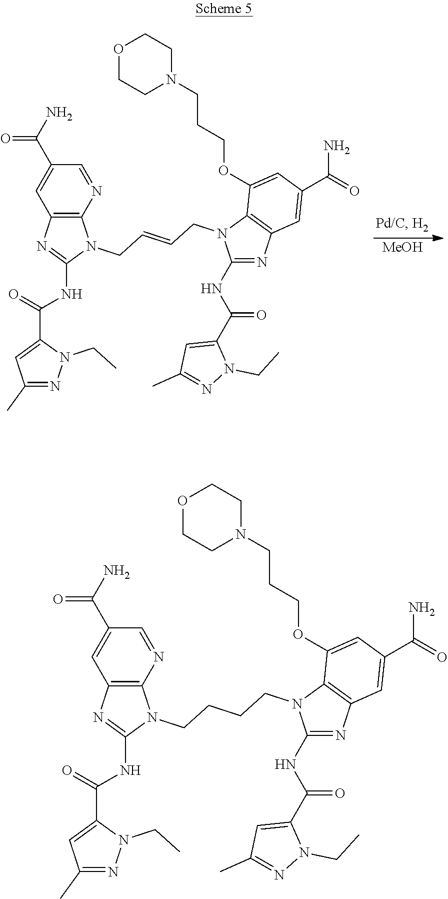 [SMILES: "MeOH" ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCOCC3)c21 ; CCn1nc(C)cc1C(=O)Nc1nc2cc(C(N)=O)cnc2n1CCCCn1c(NC(=O)c2cc(C)nn2CC)nc2cc(C(N)=O)cc(OCCCN3CCOCC3)c21]

To a solution of (E)-3-(4-(5-carbamoyl-2-(1-ethyl-3-methyl-1H-pyrazole-5-carboxamido)-7-(3-morpholinopropoxy)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide (Example 4, 500 mg, 609 umol, 1.00 eq) in MeOH (50 mL) was added Pd/C (0.5 g, 10% purity) under N₂. The suspension was degassed under vacuum and purged with H₂ several times. The mixture was stirred under H₂ (45 psi) at 50° C. for 16 hrs to give a yellow solution. LC-MS showed starting material was consumed completely and desired compound was detected. The reaction mixture was filtered and the filter was concentrated. The crude product was purified by prep-HPLC (column: Phenomenex Luna C18 100×30 mm×5 μm; mobile phase: [water (0.05% HCl)-ACN B %: 9%-39%, 9 min) to provide 3-(4-(5-carbamoyl-2-(1-ethyl-3-methyl-1H-pyrazole-5-carboxamido)-7-(3-morpholinopropoxy)-1H-benzo[d]imidazol-1-yl)butyl)-2-(1-ethyl-3-methyl-1H-pyrazole-5-carboxamido)-3H-imidazo[4,5-b]pyridine-6-carboxamide hydrochloride (300 mg, 0.341 mmol, 56.0% yield, 97.8% purity) as white solid.

¹HNMR (400 MHz, DMSO-d6): δ 11.54 (br s, 1H), 8.75 (d, J=1.9 Hz, 1H), 8.23 (br s, 1H), 8.07 (d, J=1.8 Hz, 2H), 7.48-7.68 (m, 2H), 7.39 (s, 2H), 6.63 (s, 2H), 4.56 (q, J=7.1 Hz, 8H), 4.41 (br s, 3H), 4.23 (br s, 4H), 3.69-3.98 (m, 4H), 3.34 (br d, J=11.8 Hz, 2H), 3.10-3.23 (m, 2H), 2.87-3.07 (m, 2H), 2.11 (d, J=7.1 Hz, 7H), 1.91 (br s, 4H), 1.16-1.38 (m, 6H); LCMS: m/z (ES+) [M+H]+=823.6.

### Example 6

### (E)-3-((E)-4-((E)-5-carbamoyl-2-((1-ethyl-3-methyl-1H-pyrazole-5-carbonyl)imino)-3-methyl-7-(3-morpholinopropoxy)-2,3-dihydro-1H-benzo[d]imidazol-1-yl)but-2-en-1-yl)-2-((1-ethyl-3-methyl-1H-pyrazole-5-carbonyl)imino)-1-methyl-2,3-dihydro-1H-imidazo[4,5-b]pyridine-6-carboxamide

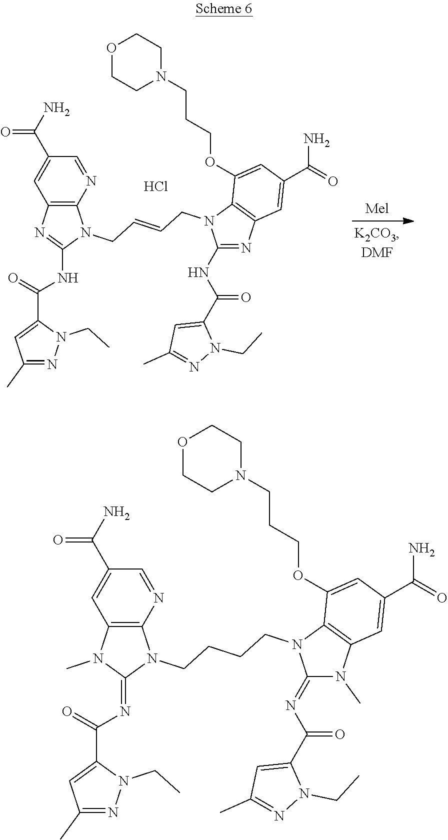 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCOCC3)c21 ; Cl ; CCn1nc(C)cc1C(=O)/N=c1\n(C)c2cc(C(N)=O)cnc2n1CCCCn1/c(=N/C(=O)c2cc(C)nn2CC)n(C)c2cc(C(N)=O)cc(OCCCN3CCOCC3)c21]

To a solution of (E)-3-(4-(5-carbamoyl-2-(1-ethyl-3-methyl-1H-pyrazole-5-carboxamido)-7-(3-morpholinopropoxy)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide (Example 4, 400 mg, 487 umol, 1.00 eq) and K₂CO₃ (148 mg, 1.07 mmol, 2.20 eq) in DMF (8 mL) was added a solution of Mel (3.05 g, 21.5 mmol, 1.34 mL, 44.1 eq) in DMF (0.8 mL) at 25° C. and the reaction stirred at 25° C. for 16 hrs to give a yellow solution. The mixture was concentrated under reduced pressure to give a residue. The crude product was purified by prep-HPLC (column: Phenomenex Gemini C18 150*25 mm*10 um; mobile phase: [water (0.05% NH₃H₂O+10 mM NH₄HCO₃)-ACN]; B %: 10%-40%, 20 min) to produce (E)-3-((E)-4-((E)-5-carbamoyl-2-((1-ethyl-3-methyl-1H-pyrazole-5-carbonyl)imino)-3-methyl-7-(3-morpholinopropoxy)-2,3-dihydro-1H-benzo[d]imidazol-1-yl)but-2-en-1-yl)-2-((1-ethyl-3-methyl-1H-pyrazole-5-carbonyl)imino)-1-methyl-2,3-dihydro-1H-imidazo[4,5-b]pyridine-6-carboxamide (120 mg, 0.138 mmol, 28.4% yield, 97.9% purity) as white solid.

¹HNMR (400 MHz, DMSO-d6): δ 8.74 (d, J=1.6 Hz, 1H), 8.35 (d, J=1.6 Hz, 1H), 8.14 (br s, 1H), 8.04 (br s, 1H), 7.71 (s, 1H), 7.61 (br s, 1H), 7.36-7.49 (m, 2H), 6.40 (s, 1H), 6.35 (s, 1H), 5.75-5.88 (m, 1H), 5.60-5.75 (m, 1H), 4.80 (br dd, J=16.4, 5.3 Hz, 4H), 4.30-4.54 (m, 4H), 4.07 (br t, J=6.3 Hz, 2H), 3.40-3.58 (m, 10H), 2.18-2.33 (m, 6H), 2.10 (d, J=13.3 Hz, 6H), 1.73 (q, J=6.5 Hz, 2H), 1.21 (dt, J=8.9, 7.2 Hz, 6H); LCMS: m/z (ES+) [M+H]+=849.6.

### Example 7

### (E)-3-((E)-4-((E)-5-carbamoyl-3-ethyl-2-((1-ethyl-3-methyl-1H-pyrazole-5-carbonyl)imino)-7-(3-morpholinopropoxy)-2,3-dihydro-1H-benzo[d]imidazol-1-yl)but-2-en-1-yl)-1-ethyl-2-((1-ethyl-3-methyl-1H-pyrazole-5-carbonyl)imino)-2,3-dihydro-1H-imidazo[4,5-b]pyridine-6-carboxamide

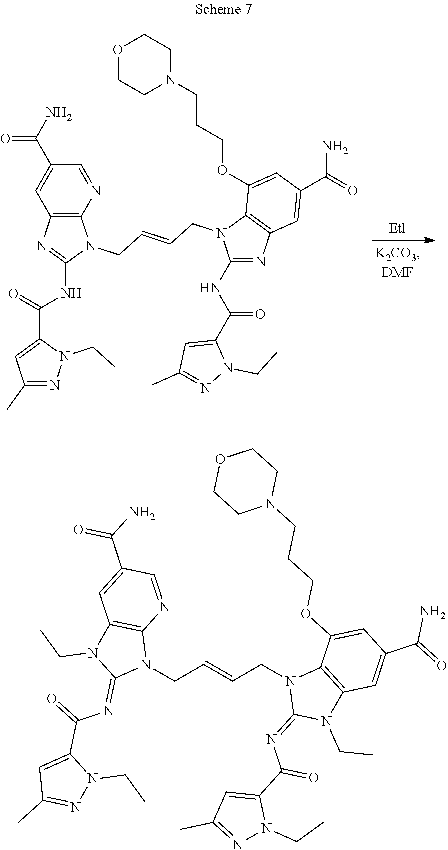 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCOCC3)c21 ; CCn1nc(C)cc1C(=O)/N=c1\n(CC)c2cc(C(N)=O)cnc2n1C/C=C/Cn1/c(=N/C(=O)c2cc(C)nn2CC)n(CC)c2cc(C(N)=O)cc(OCCCN3CCOCC3)c21]

To a suspension of (E)-3-(4-(5-carbamoyl-2-(1-ethyl-3-methyl-1H-pyrazole-5-carboxamido)-7-(3-morpholinopropoxy)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide (Example 4, 300 mg, 350 umol, 1.00 eq, HCl) in DMF (10 mL) was added cesium carbonate (431 mg, 1.32 mmol, 3.78 eq) and EtI (155 mg, 995 umol, 79.5 uL, 2.84 eq). After 3 hrs, additional ethyl iodide (40 uL*2) was added and the reaction stirred at 25° C. for 15 hrs to give a yellow solution. The reaction mixture was stirred at 0° C. for 30 min, then EDCI (4.80 g, 25.0 mmol, 3.50 eq) and Et₃N (5.80 g, 57.3 mmol, 7.97 mL, 8.00 eq) was added at 0° C., then heated to 25° C. and stirred for 16 hrs to afford a yellow solution. LC-MS showed the starting material was consumed completely and one main peak with desired mass was detected. The mixture was concentrated under reduced pressure to give a residue. The crude product was purified by prep-HPLC (column: Phenomenex Gemini C18 150*25 mm*10 um; mobile phase: [water (0.05% NH₃H₂O+10 mM NH₄HCO₃)-ACN]; B %: 10%-40%, 20 min) to provide (E)-3-((E)-4-((E)-5-carbamoyl-3-ethyl-2-((1-ethyl-3-methyl-1H-pyrazole-5-carbonyl)imino)-7-(3-morpholinopropoxy)-2,3-dihydro-1H-benzo[d]imidazol-1-yl)but-2-en-1-yl)-1-ethyl-2-((1-ethyl-3-methyl-1H-pyrazole-5-carbonyl)imino)-2,3-dihydro-1H-imidazo[4,5-b]pyridine-6-carboxamide (120 mg, 0.133 mmol, 38.0% yield, 97.3% purity) as white solid.

¹HNMR (400 MHz, DMSO-d6): δ 8.65-8.79 (m, 1H), 8.25-8.46 (m, 1H), 8.17 (br s, 1H), 8.08 (br s, 1H), 7.73 (s, 1H), 7.63 (br s, 1H), 7.47 (br s, 1H), 7.40 (s, 1H), 6.35 (s, 1H), 6.29 (s, 1H), 5.70-5.81 (m, 1H), 5.54-5.67 (m, 1H), 4.81 (br d, J=4.9 Hz, 4H), 4.28-4.50 (m, 4H), 3.98-4.21 (m, 6H), 3.49 (br t, J=4.4 Hz, 4H), 2.15-2.32 (m, 6H), 2.09 (d, J=16.4 Hz, 6H), 1.70 (quin, J=6.6 Hz, 2H), 1.08-1.28 (m, 12H); LCMS: m/z (ES+) [M+H]+=877.6.

### Example 8

### (E)-3-(4-(5-carbamoyl-2-(1-ethyl-3-methyl-1H-pyrazole-5-carboxamido)-7-(3-(piperazin-1-yl)propoxy)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide

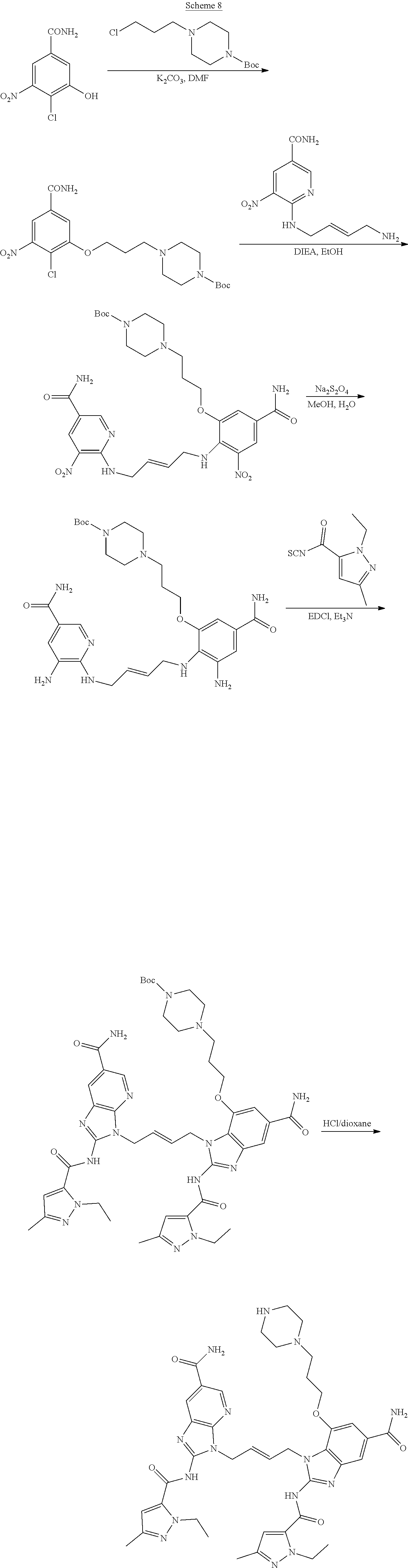

### Step 1 tert-butyl 4-(3-(5-carbamoyl-2-chloro-3-nitrophenoxy)propyl)piperazine-1-carboxylate

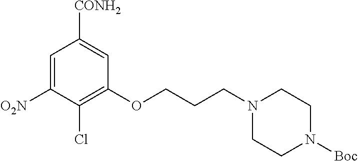 [SMILES: CC(C)(C)OC(=O)N1CCN(CCCOc2cc(C(N)=O)cc([N+](=O)[O-])c2Cl)CC1]

A mixture of 4-chloro-3-hydroxy-5-nitrobenzamide (prepared as example 3, 3.20 g, 14.8 mmol, 1.00 eq), tert-butyl 4-(3-chloropropyl)piperazine-1-carboxylate (4.66 g, 17.7 mmol, 1.20 eq) and K₂CO₃ (2.65 g, 19.2 mmol, 1.30 eq) in DMF (32 mL), the mixture was stirred at 70° C. for 16 hrs under N₂ atmosphere to give a yellow solution. LC-MS showed the starting material was consumed completely and one main peak with desired MS was detected. The mixture was concentrated under reduced pressure to give a crude product. The crude product was triturated with H₂O (100 mL) at 25° C. for 16 hrs. to provide tert-butyl 4-(3-(5-carbamoyl-2-chloro-3-nitrophenoxy)propyl)piperazine-1-carboxylate (6.00 g, 13.6 mmol, 91.7% yield) as a yellow solid.

HNMR (400 MHz, DMSO-d6): δ 8.34 (br s, 1H), 8.05 (s, 1H), 7.89 (s, 1H), 7.80 (br s, 1H), 4.28 (br t, J=5.4 Hz, 2H), 3.37 (br s, 12H), 2.17-2.38 (m, 5H), 1.86-2.08 (m, 2H), 1.39 (s, 12H); LCMS: m/z (ES+) [M+H]+=443.5.

### Step 2 tert-butyl (E)-4-(3-(5-carbamoyl-2-((4-((5-carbamoyl-3-nitropyridin-2-yl)amino)but-2-en-1-yl)amino)-3-nitrophenoxy)propyl)piperazine-1-carboxylate

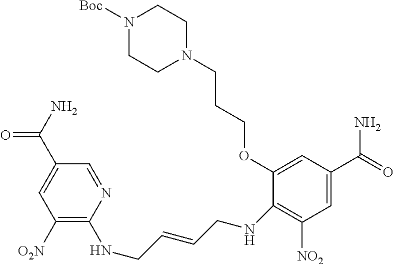 [SMILES: CC(C)(C)OC(=O)N1CCN(CCCOc2cc(C(N)=O)cc([N+](=O)[O-])c2NC/C=C/CNc2ncc(C(N)=O)cc2[N+](=O)[O-])CC1]

A solution of (E)-6-((4-aminobut-2-en-1-yl)amino)-5-nitronicotinamide (Intermediate 3, 9.74 g, 33.9 mmol, 1.50 eq, HCl), tert-butyl 4-(3-(5-carbamoyl-2-chloro-3-nitrophenoxy)propyl)piperazine-1-carboxylate (10.0 g, 22.6 mmol, 1.00 eq), DIEA (11.7 g, 90.3 mmol, 15.7 mL, 4.00 eq) and NaHCO₃ (7.59 g, 90.3 mmol, 3.51 mL, 4.00 eq) in EtOH (110 mL) was stirred at 110° C. for 16 hrs under N₂ of sealed tube to afford a yellow suspension. LC-MS showed the starting material was consumed completely and one main peak with desired MS was detected. The mixture was concentrated to give crude product. The residue was purified by flash silica gel chromatography (ISCO@; 120 g SepaFlash@ Silica Flash Column, Eluent of 5-10% Methanol/Dichloromethane @ 100 mL/min_Dichloromethane/Methanol=10:1, Rr of product=0.50, UV 254 nm) to produce tert-butyl (E)-4-(3-(5-carbamoyl-2-((4-((5-carbamoyl-3-nitropyridin-2-yl)amino)but-2-en-1-yl)amino)-3-nitrophenoxy)propyl)piperazine-1-carboxylate (5.50 g, 7.51 mmol, 33.2% yield, 89.8% purity) as red brown solid.

¹HNMR (400 MHz, DMSO-d6): δ 8.73-8.99 (m, 3H), 7.99-8.23 (m, 3H), 7.71-7.85 (m, 1H), 7.41-7.60 (m, 2H), 7.34 (br s, 1H), 5.56-5.87 (m, 2H), 4.38 (br s, 1H), 4.08-4.26 (m, 4H), 4.05 (br t, J=6.1 Hz, 2H), 3.53-3.69 (m, 1H), 3.03-3.49 (m, 15H), 2.19-2.46 (m, 6H), 1.90 (br s, 2H), 1.39 (s, 9H), 1.17-1.31 (m, 5H), 1.06 (t, J=7.0 Hz, 3H); LCMS: m/z (ES+) [M+H]+=658.2.

### Step 3 tert-butyl (E)-4-(3-(3-amino-2-((4-((3-amino-5-carbamoylpyridin-2-yl)amino)but-2-en-1-yl)amino)-5-carbamoylphenoxy)propyl)piperazine-1-carboxylate

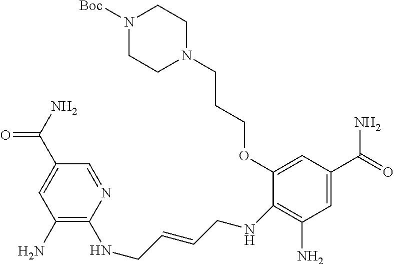 [SMILES: CC(C)(C)OC(=O)N1CCN(CCCOc2cc(C(N)=O)cc(N)c2NC/C=C/CNc2ncc(C(N)=O)cc2N)CC1]

To a solution of tert-butyl (E)-4-(3-(5-carbamoyl-2-((4-((5-carbamoyl-3-nitropyridin-2-yl)amino)but-2-en-1-yl)amino)-3-nitrophenoxy)propyl)piperazine-1-carboxylate (5.50 g, 8.36 mmol, 1.00 eq) in MeOH (110 mL) and H₂O (55.0 mL) was added NaHCO₃ (42.0 g, 500 mmol, 19.5 mL, 59.8 eq) followed by disodium;BLAH (20.4 g, 117 mmol, 25.5 mL, 14.0 eq) at 0° C. and then the reaction mixture was stirred at 20° C. for 2 hrs. A light yellow suspension was obtained. LCMS showed Reactant 1 was consumed completely and one main peak with desired MS was detected. The reaction mixture was filtered and the filter cake was washed with MeOH (100 mL*2). The filtrate was concentrated. The crude product tert-butyl (E)-4-(3-(3-amino-2-((4-((3-amino-5-carbamoylpyridin-2-yl)amino)but-2-en-1-yl)amino)-5-carbamoylphenoxy)propyl)piperazine-1-carboxylate (5.00 g, 8.37 mmol, 100% yield) was used directly in the next step without purification. LCMS: m/z (ES+) [M+H]+=598.2;

### Step 4 tert-butyl (E)-4-(3-((5-carbamoyl-1-(4-(6-carbamoyl-2-(1-ethyl-3-methyl-1H-pyrazole-5-carboxamido)-3H-imidazo[4,5-b]pyridin-3-yl)but-2-en-1-yl)-2-(1-ethyl-3-methyl-1H-pyrazole-5-carboxamido)-1H-benzo[d]imidazol-7-yl)oxy)propyl)piperazine-1-carboxylate

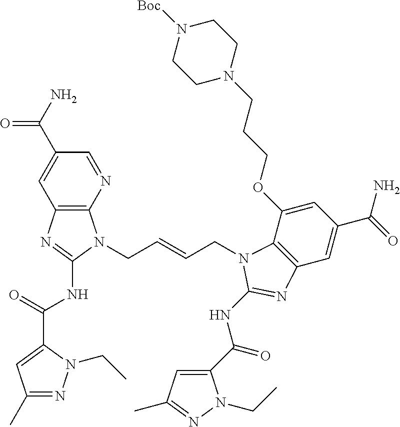 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCN(C(=O)OC(C)(C)C)CC3)c21]

To a solution of tert-butyl (E)-4-(3-(3-amino-2-((4-((3-amino-5-carbamoylpyridin-2-yl)amino)but-2-en-1-yl)amino)-5-carbamoylphenoxy)propyl)piperazine-1-carboxylate (5.00 g, 8.37 mmol, 1.00 eq) in DMF (100 mL) was added a solution of 1-ethyl-3-methyl-1H-pyrazole-5-carbonyl isothiocyanate (Intermediate 4, 4.41 g, 22.6 mmol, 2.70 eq) in dioxane (20 mL) of 0 min (3 mL), 10 min (3 mL), 15 min (3 mL) at 0° C., respectively. The reaction mixture was stirred at 0° C. for 30 min, then EDCI (5.61 g, 29.3 mmol, 3.50 eq) and Et₃N (6.77 g, 66.9 mmol, 9.31 mL, 8.00 eq) was added at 0° C., then heated to 25° C. and stirred for 2 hrs to afford a yellow solution. LC-MS showed the starting material was consumed completely and desired compound was detected. The reaction mixture was quenched with a saturated aqueous NH₄Cl solution (50 mL). The solution was filtered and the filtrate was concentrated. The crude product was purified by prep-HPLC (column: Phenomenex Gemini YMC Triart C18 250*50 mm*7 um; mobile phase: [water (10 mM NH4OAc)-ACN, 30-49, 30 min) to provide tert-butyl (E)-4-(3-((5-carbamoyl-1-(4-(6-carbamoyl-2-(1-ethyl-3-methyl-1H-pyrazole-5-carboxamido)-3H-imidazo[4,5-b]pyridin-3-yl)but-2-en-1-yl)-2-(1-ethyl-3-methyl-1H-pyrazole-5-carboxamido)-1H-benzo[d]imidazol-7-yl)oxy)propyl)piperazine-1-carboxylate (2.50 g, 2.42 mmol, 28.9% yield, 89.0% purity) as white solid. LCMS: m/z (ES+) [M+H]⁺=920.5.

### Step 5 (E)-3-(4-(5-carbamoyl-2-(1-ethyl-3-methyl-1H-pyrazole-5-carboxamido)-7-(3-(piperazin-1-yl)propoxy)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide

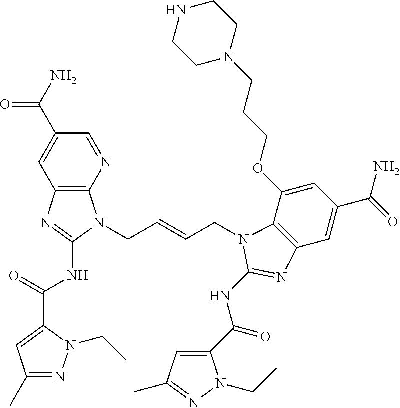 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCNCC3)c21]

To a solution of tert-butyl (E)-4-(3-((5-carbamoyl-1-(4-(6-carbamoyl-2-(1-ethyl-3-methyl-1H-pyrazole-5-carboxamido)-3H-imidazo[4,5-b]pyridin-3-yl)but-2-en-1-yl)-2-(1-ethyl-3-methyl-1H-pyrazole-5-carboxamido)-1H-benzo[d]imidazol-7-yl)oxy)propyl)piperazine-1-carboxylate (900 mg, 978 μmol, 1.00 eq) in MeOH (15 mL) and dioxane (15 mL) was added HCl/dioxane (4 M, 30 mL, 123 eq) and the mixture was stirred at 25° C. for 16 hrs under N₂ to afford a yellow solution. LC-MS showed the starting material was consumed completely and desired compound was detected. The mixture was concentrated under reduced pressure to give a residue. The crude product was purified by prep-HPLC (column: Phenomenex Gemini YMC Triart C18 250×50 mm×7 μm; mobile phase: [water (0.05% HCl)-ACN, 20-80 30 min) to provide (E)-3-(4-(5-carbamoyl-2-(1-ethyl-3-methyl-1H-pyrazole-5-carboxamido)-7-(3-(piperazin-1-yl)propoxy)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide (150 mg, 0.176 mmol, 17.9% yield, 96.0% purity) as white solid.

¹HNMR (400 MHz, DMSO-d6): δ 11.89-12.32 (m, 1H), 9.57-10.02 (m, 2H), 8.78 (d, J=1.8 Hz, 1H), 8.09-8.33 (m, 2H), 7.99 (br s, 1H), 7.66 (d, J=1.0 Hz, 1H), 7.56 (br s, 1H), 7.26-7.49 (m, 2H), 6.53 (s, 1H), 6.48 (s, 1H), 6.00 (dt, J=15.5, 5.1 Hz, 1H), 5.73 (dt, J=15.6, 5.6 Hz, 1H), 4.98 (br d, J=3.6 Hz, 2H), 4.81 (br d, J=5.3 Hz, 2H), 4.39-4.58 (m, 4H), 3.21-3.80 (m, 9H), 2.13-2.30 (m, 3H), 2.09 (d, J=4.9 Hz, 6H), 1.25 ppm (td, J=7.1, 3.9 Hz, 6H); LCMS: m/z (ES+) [M+H]+=920.4.

### Example 9

### 3-((E)-4-(5-carbamoyl-7-(3-(4-methylpiperazin-1-yl)propoxy)-2-((E)-pent-2-enamido)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide

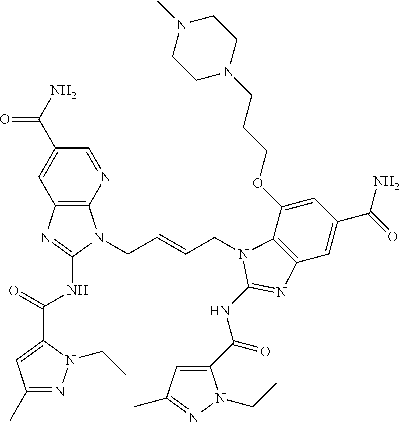 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCN(C)CC3)c21]

To a solution of (E)-3-(4-(5-carbamoyl-2-(1-ethyl-3-methyl-1H-pyrazole-5-carboxamido)-7-(3-(piperazin-1-yl)propoxy)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide (1.50 g, 1.75 mmol, 1.00 eq, HCl) in MeOH (50 mL) was added TEA (291 mg, 2.87 mmol, 400 uL, 1.64 eq) and the mixture was stirred at 15° C. for 15 min. Then to the mixture was added AcOH (137 mg, 2.28 mmol, 130 uL, 1.30 eq) followed by sodium;triacetoxyboranuide (3.35 g, 15.8 mmol, 9.02 eq) and (CHO)n (1.35 g, 1.50 eq) below 20° C. and the reaction mixture was stirred at 28° C. for 12 hrs. A white suspension was obtained. LC-MS showed the starting material was consumed completely and desired compound was detected. The mixture was concentrated under reduced pressure to give a residue. The crude product was purified by prep-HPLC (column: Phenomenex Gemini YMC Triart C18 250×50 mm×7 μm; mobile phase: [water (0.05% HCl)-ACN, 30-49 30 min) to provide 3-((E)-4-(5-carbamoyl-7-(3-(4-methylpiperazin-1-yl)propoxy)-2-((E)-pent-2-enamido)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide (450 mg, 500 umol, 28.5% yield, 96.8% purity, HCl) as a white solid.

¹HNMR (400 MHz, DMSO-d6): δ 12.05 (br s, 2H), 8.79 (s, 1H), 7.89-8.27 (m, 3H), 7.61-7.72 (m, 1H), 7.57 (br s, 1H), 7.40 (s, 2H), 6.40-6.68 (m, 2H), 6.01 (br d, J=15.5 Hz, 1H), 5.61-5.80 (m, 1H), 5.00 (br s, 2H), 4.82 (br d, J=4.1 Hz, 4H), 4.37-4.52 (m, 8H), 4.20 (br s, 2H), 3.21-3.90 (m, 10H), 2.85 (br s, 3H), 2.13-2.28 (m, 2H), 2.09 (d, J=5.1 Hz, 6H), 1.14-1.36 (m, 6H); LCMS: m/z (ES+) [M+H]+=834.5.

### Example 10

### (E)-3-(4-(5-carbamoyl-2-(1-ethyl-3-methyl-1H-pyrazole-5-carboxamido)-7-(2-morpholinoethoxy)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide

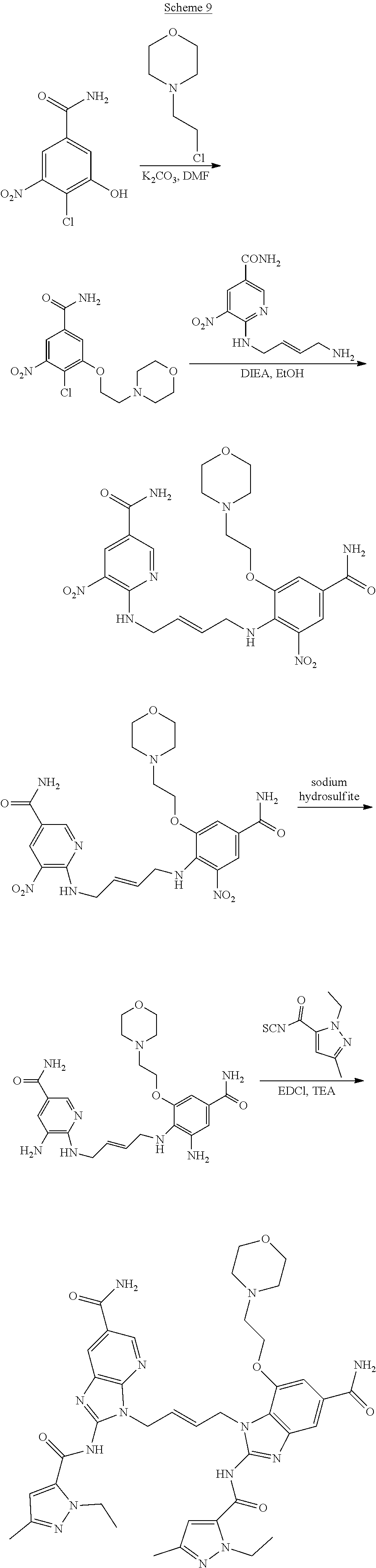

### Step 1 4-chloro-3-(2-morpholinoethoxy)-5-nitrobenzamide

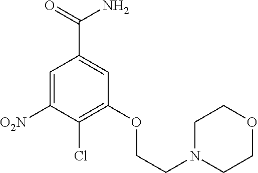 [SMILES: NC(=O)c1cc(OCCN2CCOCC2)c(Cl)c([N+](=O)[O-])c1]

A mixture of 4-chloro-3-hydroxy-5-nitrobenzamide (prepared as example 3, 1.00 g, 4.62 mmol, 1.00 eq), 4-(2-chloroethyl)morpholine (1.03 g, 5.54 mmol, 1.20 eq, HCl) and K₂CO₃ (1.28 g, 9.23 mmol, 2.00 eq) in DMF (15.0 mL), the mixture was stirred at 70° C. for 16 hrs under N₂ atmosphere to give a yellow solution. LC-MS showed the starting material was consumed completely and one main peak with desired MS was detected. The mixture was concentrated under reduced pressure to give a crude product. The crude product was triturated with H₂O (50.0 mL) at 25° C. for 16 hrs. 4-chloro-3-(2-morpholinoethoxy)-5-nitrobenzamide (2.60 g, 7.65 mmol, 82.8% yield, 97.0% purity) was obtained as a yellow solid. LCMS: m/z (ES+) [M+H]+=330.1.

### Step 2 (E)-6-((4-((4-carbamoyl-2-(2-morpholinoethoxy)-6-nitrophenyl)amino)but-2-en-1-yl)amino)-5-nitronicotinamide

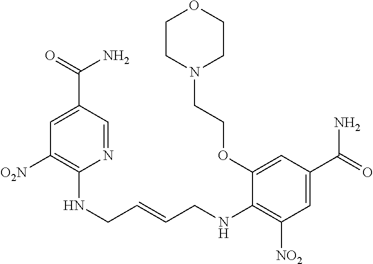 [SMILES: NC(=O)c1cnc(NC/C=C/CNc2c(OCCN3CCOCC3)cc(C(N)=O)cc2[N+](=O)[O-])c([N+](=O)[O-])c1]

A solution of 4-chloro-3-(2-morpholinoethoxy)-5-nitrobenzamide (1.05 g, 3.64 mmol, 1.20 eq, HCl), NaHCO₃ (1.02 g, 12.1 mmol, 472 uL, 4.00 eq) and 4-chloro-3-(2-morpholinoethoxy)-5-nitro-benzamide (Intermediate 3, 1.00 g, 3.03 mmol, 1.00 eq), DIEA (1.57 g, 12.1 mmol, 2.11 mL, 4.00 eq) in EtOH (10.0 mL) was stirred at 110° C. for 16 hrs under N₂ to afford a yellow suspension. LCMS showed the starting material was consumed completely. The residue was purified by flash silica gel chromatography (ISCO@; 330 g SepaFlash@ Silica Flash Column, Eluent of 2-5% Methanol/Dichloromethane @ 100 mL/min Dichloromethane/Methanol=10:1, R_{f} of product=0.27, UV 254 nm). (E)-6-((4-((4-carbamoyl-2-(2-morpholinoethoxy)-6-nitrophenyl)amino)but-2-en-1-yl)amino)-5-nitronicotinamide (1.34 g, 2.12 mmol, 69.8% yield) was obtained as a red brown solid.

¹HNMR (400 MHz, DMSO-d6): δ 0.77-0.85 (m, 6H) 1.11 (t, J=7.13 Hz, 4H) 3.49 (br d, J=6.13 Hz, 2H) 3.75-4.25 (m, 12H) 5.56-5.74 (m, 2H) 6.82-6.96 (m, 1H) 7.78 (br t, J=6.00 Hz, 1H) 7.93-8.15 (m, 3H) 8.72-8.91 (m, 3H); LCMS: m/z (ES+) [M+H]⁺=545.2.

### Step 3 (E)-5-amino-6-((4-((2-amino-4-carbamoyl-6-(2-morpholinoethoxy)phenyl)amino)but-2-en-1-yl)amino)nicotinamide

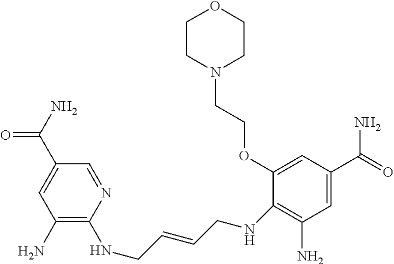 [SMILES: NC(=O)c1cnc(NC/C=C/CNc2c(N)cc(C(N)=O)cc2OCCN2CCOCC2)c(N)c1]

To a solution of (E)-6-((4-((4-carbamoyl-2-(2-morpholinoethoxy)-6-nitrophenyl)amino)but-2-en-1-yl)amino)-5-nitronicotinamide (1.00 g, 1.84 mmol, 1.00 eq) in MeOH (20 mL) and H₂O (15.0 mL) was added NaHCO₃ (9.00 g, 107 mmol, 4.17 mL, 58.3 eq) followed by disodium;BLAH (4.48 g, 25.7 mmol, 5.60 mL, 14.0 eq) at 0° C. and then the reaction mixture was stirred at 20° C. for 2 hrs. A light yellow suspension was obtained. LCMS showed the starting material was consumed completely and one main peak with desired MS was detected. The reaction mixture was filtered and the filter cake was washed with MeOH (50.0 mL). The filtrate was concentrated. The crude product was used directly in the next step without purification. (E)-5-amino-6-((4-((2-amino-4-carbamoyl-6-(2-morpholinoethoxy)phenyl)amino)but-2-en-1-yl)amino)nicotinamide (889 mg, 1.84 mmol, 100% yield) was obtained as a yellow solid. LCMS: m/z (ES+) [M+H]⁺=484.3.

### Step 4 (E)-3-(4-(5-carbamoyl-2-(1-ethyl-3-methyl-1H-pyrazole-5-carboxamido)-7-(2-morpholinoethoxy)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide

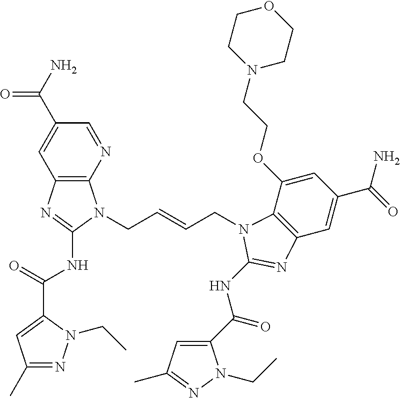 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCN3CCOCC3)c21]

To a solution of the (E)-5-amino-6-((4-((2-amino-4-carbamoyl-6-(2-morpholinoethoxy)phenyl)amino)but-2-en-1-yl)amino)nicotinamide (889 mg, 1.84 mmol, 1.00 eq), in DMF (23.0 mL) was added a solution of 2-ethyl-5-methyl-pyrazole-3-carbonyl isothiocyanate (Intermediate 4, 968 mg, 4.96 mmol, 2.70 eq) in dioxane (5.30 mL) of 0 min (3.00 mL), 10 min (3.00 mL), 15 min (3.00 mL) at 0° C., respectively. The reaction mixture was stirred at 0° C. for 35 mins, then EDCI (1.23 g, 6.43 mmol, 3.50 eq) and Et₃N (1.49 g, 14.7 mmol, 2.04 mL, 8.00 eq) was added at 0° C., then heated to 25° C. and stirred for 16 hrs to afford a yellow solution. LCMS showed Reactant 1 was consumed completely and desired compound was detected. The reaction mixture was quenched with a saturated aqueous NH₄Cl solution (50.0 mL). The solution was filtered and the filtrate was concentrated. The crude product was purified by prep-HPLC (column: Phenomenex Gemini C18 250×50 mm×7 um; mobile phase: [water (10 mM HCl)-ACN]; B %: 10%-40%, 20 min) to provide (E)-3-(4-(5-carbamoyl-2-(1-ethyl-3-methyl-1H-pyrazole-5-carboxamido)-7-(2-morpholinoethoxy)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide (52.0 mg, 0.06 mmol, 3.27% yield) as a white solid.

¹HNMR (400 MHz, DMSO-d6): δ 1.19-1.36 (m, 8H) 2.03-2.25 (m, 9H) 2.26-2.35 (m, 5H) 2.68 (br s, 1H) 3.45-3.53 (m, 3H) 4.11 (br t, J=5.63 Hz, 2H) 4.45-4.61 (m, 4H) 4.79 (br s, 2H) 4.97 (br s, 2H) 5.87-5.99 (m, 2H) 6.54 (s, 2H) 7.34 (s, 2H) 7.53 (br s, 1H) 7.64 (s, 1H) 7.93 (br s, 1H) 8.12-8.19 (m, 2H) 8.71 (s, 1H); LCMS: m/z (ES+) [M+H]+=807.3.

### Example 11

### (E)-3-(4-(5-carbamoyl-2-(1,5-dimethyl-1H-pyrazole-3-carboxamido)-7-(3-morpholinopropoxy)-1H-benzo[d]imidazol-1-yl)but-2-en-1-yl)-2-(1,5-dimethyl-1H-pyrazole-3-carboxamido)-3H-imidazo[4,5-b]pyridine-6-carboxamide

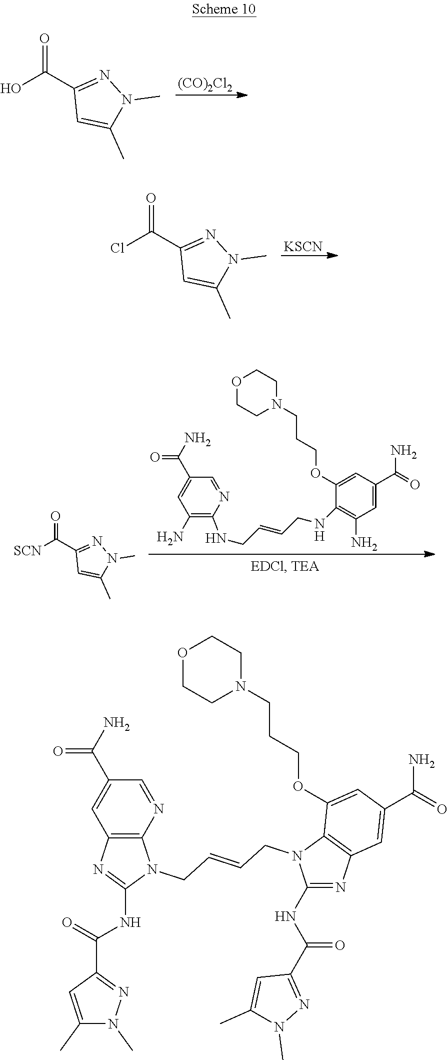

### Step 1 1,5-dimethyl-1H-pyrazole-3-carbonyl chloride

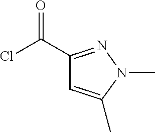

To a solution of 1,5-dimethyl-1H-pyrazole-3-carboxylic acid (4.00 g, 28.5 mmol) in DCM (80 mL) and DMF (83.6 mg, 1.14 mmol, 88.0 uL) was added drops of oxalyl dichloride (4.35 g, 34.3 mmol, 3.00 mL) slowly at 10-25° C. and the reaction mixture was stirred at 25° C. for 1 hr under N₂ to give a yellow solution. TLC (Petroleum ether/EtOAc=3/1, R_{f}=0.24, UV) showed one main spot with lower polarity was detected. The reaction was clean according to TLC. The mixture was concentrated under reduced pressure to give a residue. The residue was added 1,2-dichloroethane (50 mL)*3 to remove oxalyl dichloride under reduced pressure afford crude product. The crude product was used directly in the next step without purification. 1,5-dimethyl-1H-pyrazole-3-carbonyl chloride (4.20 g, 26.5 mmol, 92.7% yield) was obtained as yellow solid.

### Step 2 1,5-dimethyl-1H-pyrazole-3-carbonyl isothiocyanate

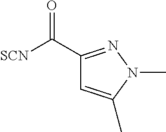

To a dry 1 L round bottom flask was added KSCN (3.35 g, 34.4 mmol, 3.35 mL, 1.30 eq) and acetone (80 mL). This clear homogenous solution was cooled to 0° C. After 5 min, stirring at 0° C. 1,5-dimethylpyrazole-3-carbonyl chloride (4.20 g, 26.5 mmol, 1.00 eq) was added as a solution in ACETONE (16 mL). Once the addition was complete, the reaction was allowed to stirring at 0° C. After 1 min, additional KSCN (1.34 g, 13.8 mmol, 1.34 mL, 5.21e-1 eq) and the reaction was stirred for an additional 20 min to give a yellow solution. TLC (Petroleum ether/EtOAc=3/1, Rr=0.40, UV) showed one main spot with lower polarity was detected. The reaction was clean according to TLC. Hexane (100 mL) was added to the reaction mixture. The reaction mixture was filtered and the filter cake was washed with hexane (50 mL)*2). The organic phase was concentrated to give a residue. Then, hexane (100 mL) and Ethyl acetate (100 mL) was added to the residue. The crude mixture was filtered and the filtrate was concentrated in vacuum. The crude product was used directly in the next step without purification. 1,5-dimethyl-1H-pyrazole-3-carbonyl isothiocyanate (4.50 g, 24.8 mmol, 93.7% yield) was obtained as yellow oil.

### Step 3 (E)-3-(4-(5-carbamoyl-2-(1,5-dimethyl-1H-pyrazole-3-carboxamido)-7-(3-morpholinopropoxy)-1H-benzo[d]imidazol-1-yl)but-2-en-1-yl)-2-(1,5-dimethyl-1H-pyrazole-3-carboxamido)-3H-imidazo[4,5-b]pyridine-6-carboxamide

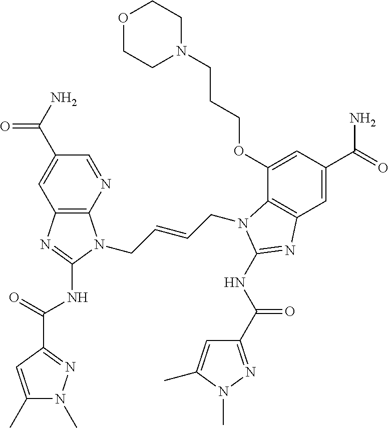 [SMILES: Cc1cc(C(=O)Nc2nc3cc(C(N)=O)cnc3n2C/C=C/Cn2c(NC(=O)c3cc(C)n(C)n3)nc3cc(C(N)=O)cc(OCCCN4CCOCC4)c32)nn1C]

To a solution of (E)-5-amino-6-((4-((2-amino-4-carbamoyl-6-(3-morpholinopropoxy)phenyl)amino)but-2-en-1-yl)amino)nicotinamide (Prepared as Example 4, 3.57 g, 7.16 mmol, 1.00 eq) in DMF (80 mL) was added a solution of 1,5-dimethylpyrazole-3-carbonyl isothiocyanate (3.50 g, 19.3 mmol, 2.70 eq) in dioxane (8 mL) of 0 min (3 mL), 10 min (3 mL), 15 min (3 mL) at 0° C., respectively. The reaction mixture was stirred at 0° C. for 30 min, then EDCI (4.80 g, 25.0 mmol, 3.50 eq) and Et₃N (5.80 g, 57.3 mmol, 7.97 mL, 8.00 eq) was added at 0° C., then heated to 25° C. and stirred for 16 hrs to afford a yellow solution. LC-MS showed the starting material was consumed completely and desired compound was detected. The reaction mixture was quenched with a saturated aqueous NH₄Cl solution (100 mL). The solution was filtered and the filtrate was concentrated. The crude product was purified by prep-HPLC (column: Phenomenex Luna C18 100*30 mm*5 um; mobile phase: [water (0.05%NH₃H₂O+10 mM NH₄HCO₃)-ACN]; B %: 0%-30%, 9 min) to provide (E)-3-(4-(5-carbamoyl-2-(1,5-dimethyl-1H-pyrazole-3-carboxamido)-7-(3-morpholinopropoxy)-1H-benzo[d]imidazol-1-yl)but-2-en-1-yl)-2-(1,5-dimethyl-1H-pyrazole-3-carboxamido)-3H-imidazo[4,5-b]pyridine-6-carboxamide (300 mg, 0.368 mmol, 5.14% yield, 97.3% purity) as white solid.

¹HNMR (400 MHz, DMSO-d6): δ=12.45-12.83 (m, 1H), 10.36 (br s, 1H), 8.71 (br s, 1H), 8.26 (br s, 1H), 8.11 (br s, 1H), 7.94 (br s, 1H), 7.55-7.83 (m, 1H), 7.50 (br s, 1H), 7.11-7.40 (m, 2H), 6.23-6.56 (m, 2H), 5.52-6.00 (m, 2H), 4.61-5.05 (m, 4H), 3.88-4.22 (m, 2H), 3.62-3.88 (m, 7H), 3.41-3.60 (m, 5H), 2.01-2.36 (m, 14H), 1.49-1.87 (m, 1H), 1.45-1.80 (m, 2H), 1.24 ppm (br s, 1H); LCMS: m/z (ES+) [M+H]⁺=793.5.

### Example 12-46

Following a similar procedure of Examples, the following compounds were synthesized via reaction of the appropriate amine and acid derivative.

| | | | LCMS m/z |
|---|---|---|---|
| Example | Structure | Method | (M + H⁺) |
| 12 | 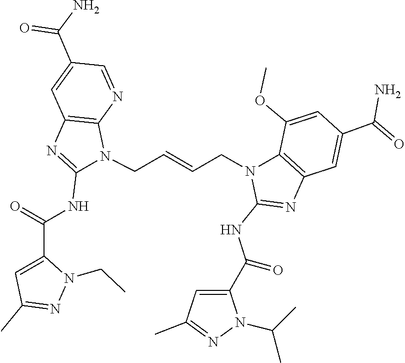 [SMILES: CCn1nc(C)cc1C(=O)Nc1nc2cc(C(N)=O)cnc2n1C/C=C/Cn1c(NC(=O)c2cc(C)nn2C(C)C)nc2cc(C(N)=O)cc(OC)c21] | Example 2 | 722 |
| 13 | 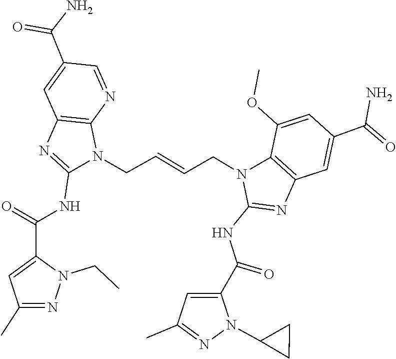 [SMILES: CCn1nc(C)cc1C(=O)Nc1nc2cc(C(N)=O)cnc2n1C/C=C/Cn1c(NC(=O)c2cc(C)nn2C2CC2)nc2cc(C(N)=O)cc(OC)c21] | Example 2 | 720 |
| 14 | 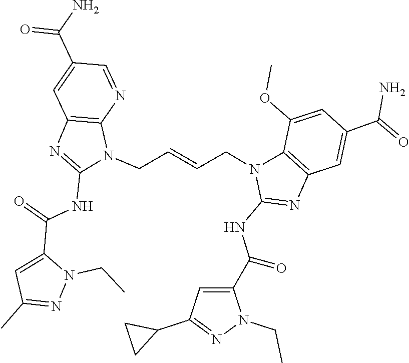 [SMILES: CCn1nc(C)cc1C(=O)Nc1nc2cc(C(N)=O)cnc2n1C/C=C/Cn1c(NC(=O)c2cc(C3CC3)nn2CC)nc2cc(C(N)=O)cc(OC)c21] | Example 2 | 734 |
| 15 | 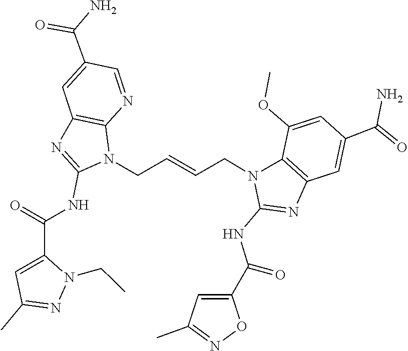 [SMILES: CCn1nc(C)cc1C(=O)Nc1nc2cc(C(N)=O)cnc2n1C/C=C/Cn1c(NC(=O)c2cc(C)no2)nc2cc(C(N)=O)cc(OC)c21] | Example 2 | 681 |
| 16 | 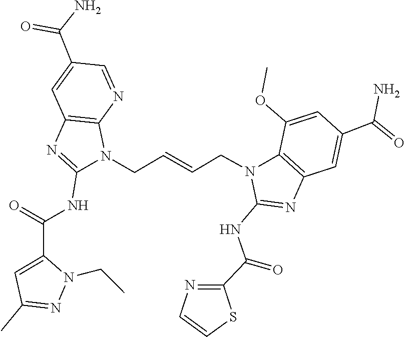 [SMILES: CCn1nc(C)cc1C(=O)Nc1nc2cc(C(N)=O)cnc2n1C/C=C/Cn1c(NC(=O)c2nccs2)nc2cc(C(N)=O)cc(OC)c21] | Example 2 | 683 |
| 17 | 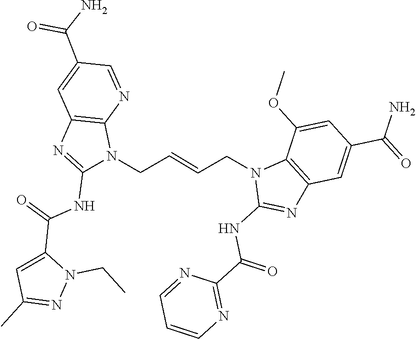 [SMILES: CCn1nc(C)cc1C(=O)Nc1nc2cc(C(N)=O)cnc2n1C/C=C/Cn1c(NC(=O)c2ncccn2)nc2cc(C(N)=O)cc(OC)c21] | Example 2 | 678 |
| 18 | 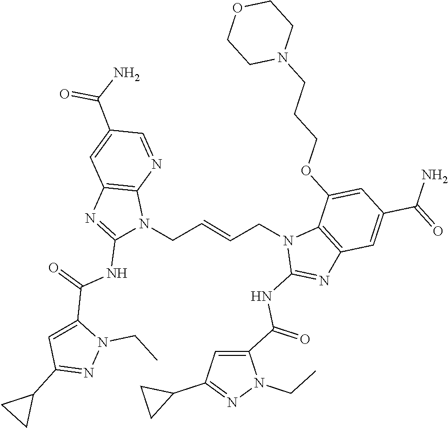 [SMILES: CCn1nc(C2CC2)cc1C(=O)Nc1nc2cc(C(N)=O)cnc2n1C/C=C/Cn1c(NC(=O)c2cc(C3CC3)nn2CC)nc2cc(C(N)=O)cc(OCCCN3CCOCC3)c21] | Example 4 | 873 |
| 19 | 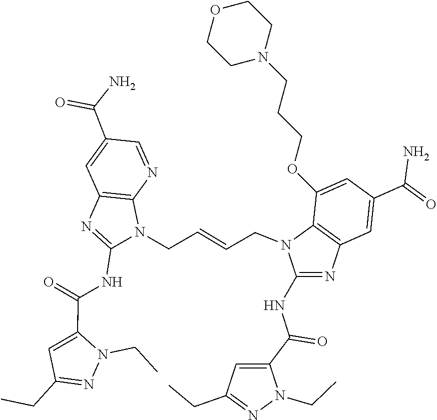 [SMILES: CCc1cc(C(=O)Nc2nc3cc(C(N)=O)cnc3n2C/C=C/Cn2c(NC(=O)c3cc(CC)nn3CC)nc3cc(C(N)=O)cc(OCCCN4CCOCC4)c32)n(CC)n1] | Example 4 | 849 |
| 20 | 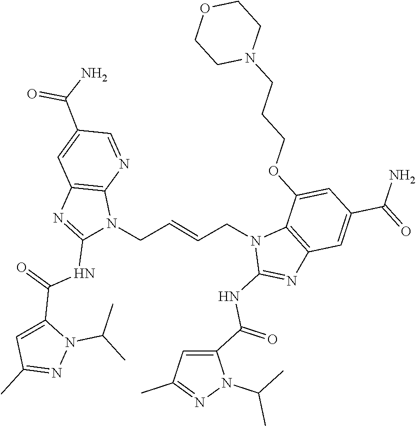 [SMILES: Cc1cc(C(=O)Nc2nc3cc(C(N)=O)cnc3n2C/C=C/Cn2c(NC(=O)c3cc(C)nn3C(C)C)nc3cc(C(N)=O)cc(OCCCN4CCOCC4)c32)n(C(C)C)n1] | Example 4 | 849 |
| 21 | 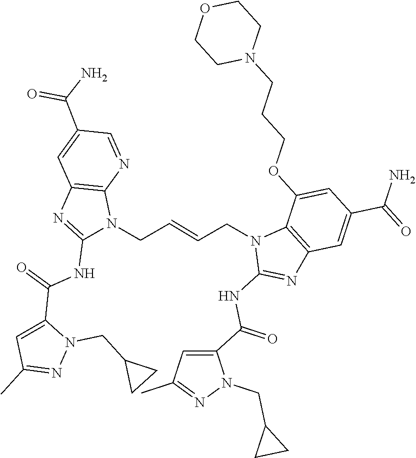 [SMILES: Cc1cc(C(=O)Nc2nc3cc(C(N)=O)cnc3n2C/C=C/Cn2c(NC(=O)c3cc(C)nn3CC3CC3)nc3cc(C(N)=O)cc(OCCCN4CCOCC4)c32)n(CC2CC2)n1] | Example 4 | 873 |
| 22 | 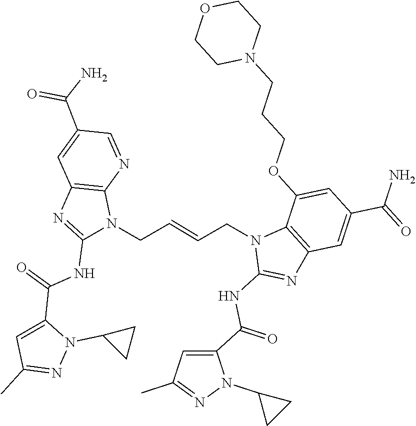 [SMILES: Cc1cc(C(=O)Nc2nc3cc(C(N)=O)cnc3n2C/C=C/Cn2c(NC(=O)c3cc(C)nn3C3CC3)nc3cc(C(N)=O)cc(OCCCN4CCOCC4)c32)n(C2CC2)n1] | Example 4 | 845 |
| 23 | 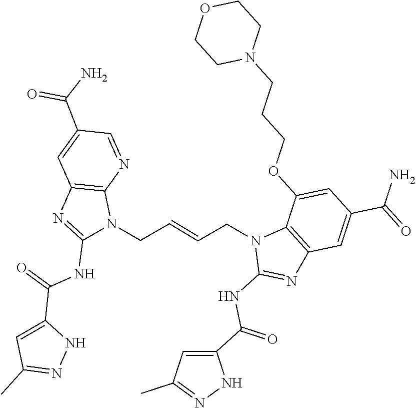 [SMILES: Cc1cc(C(=O)Nc2nc3cc(C(N)=O)cnc3n2C/C=C/Cn2c(NC(=O)c3cc(C)n[nH]3)nc3cc(C(N)=O)cc(OCCCN4CCOCC4)c32)[nH]n1] | Example 4 | 765 |
| 24 | 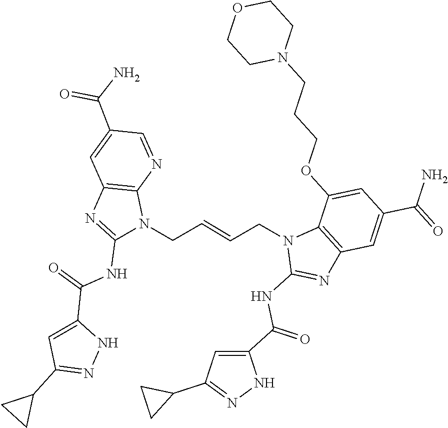 [SMILES: NC(=O)c1cnc2c(c1)nc(NC(=O)c1cc(C3CC3)n[nH]1)n2C/C=C/Cn1c(NC(=O)c2cc(C3CC3)n[nH]2)nc2cc(C(N)=O)cc(OCCCN3CCOCC3)c21] | Example 4 | 817 |
| 25 | 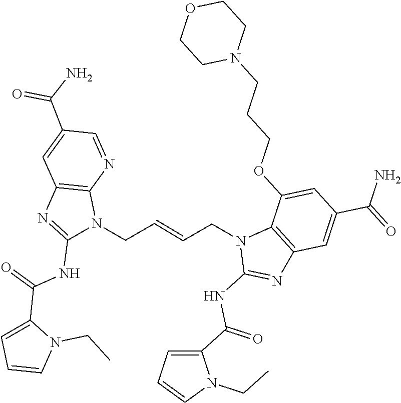 [SMILES: CCn1cccc1C(=O)Nc1nc2cc(C(N)=O)cnc2n1C/C=C/Cn1c(NC(=O)c2cccn2CC)nc2cc(C(N)=O)cc(OCCCN3CCOCC3)c21] | Example 4 | 791 |
| 26 | 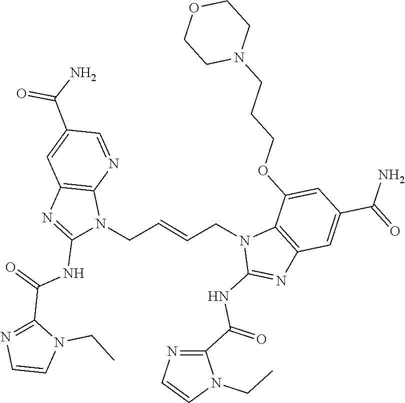 [SMILES: CCn1ccnc1C(=O)Nc1nc2cc(C(N)=O)cnc2n1C/C=C/Cn1c(NC(=O)c2nccn2CC)nc2cc(C(N)=O)cc(OCCCN3CCOCC3)c21] | Example 4 | 794 |
| 27 | 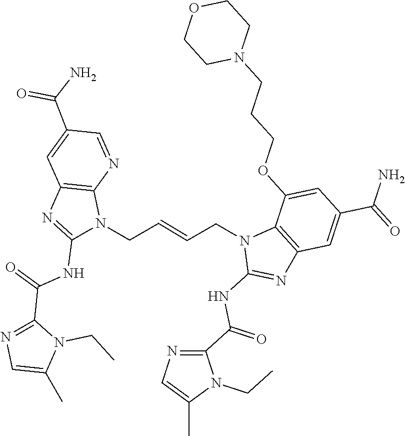 [SMILES: CCn1c(C)cnc1C(=O)Nc1nc2cc(C(N)=O)cnc2n1C/C=C/Cn1c(NC(=O)c2ncc(C)n2CC)nc2cc(C(N)=O)cc(OCCCN3CCOCC3)c21] | Example 4 | 822 |
| 28 | 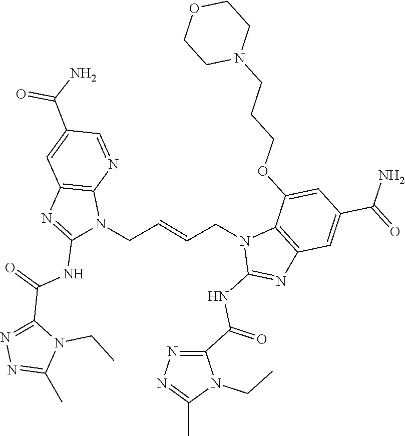 [SMILES: CCn1c(C)nnc1C(=O)Nc1nc2cc(C(N)=O)cnc2n1C/C=C/Cn1c(NC(=O)c2nnc(C)n2CC)nc2cc(C(N)=O)cc(OCCCN3CCOCC3)c21] | Example 4 | 824 |
| 29 | 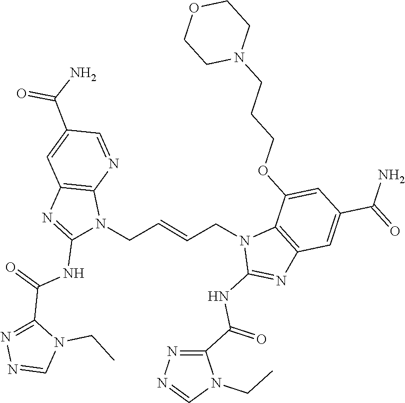 [SMILES: CCn1cnnc1C(=O)Nc1nc2cc(C(N)=O)cnc2n1C/C=C/Cn1c(NC(=O)c2nncn2CC)nc2cc(C(N)=O)cc(OCCCN3CCOCC3)c21] | Example 4 | 795 |
| 30 | 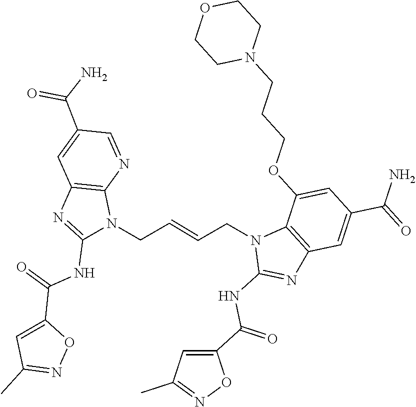 [SMILES: Cc1cc(C(=O)Nc2nc3cc(C(N)=O)cnc3n2C/C=C/Cn2c(NC(=O)c3cc(C)no3)nc3cc(C(N)=O)cc(OCCCN4CCOCC4)c32)on1] | Example 4 | 767 |
| 31 | 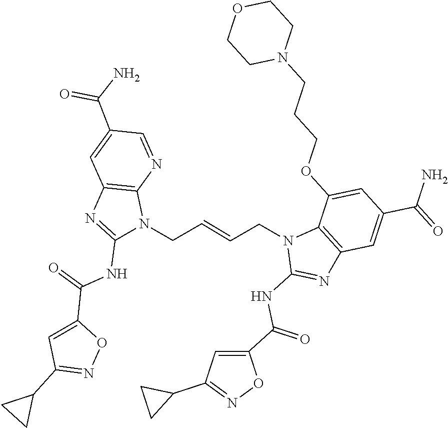 [SMILES: NC(=O)c1cnc2c(c1)nc(NC(=O)c1cc(C3CC3)no1)n2C/C=C/Cn1c(NC(=O)c2cc(C3CC3)no2)nc2cc(C(N)=O)cc(OCCCN3CCOCC3)c21] | Example 4 | 819 |
| 32 | 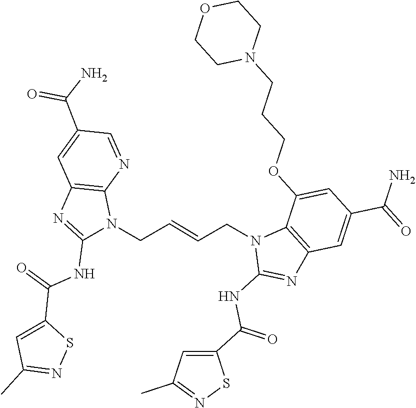 [SMILES: Cc1cc(C(=O)Nc2nc3cc(C(N)=O)cnc3n2C/C=C/Cn2c(NC(=O)c3cc(C)ns3)nc3cc(C(N)=O)cc(OCCCN4CCOCC4)c32)sn1] | Example 4 | 799 |
| 33 | 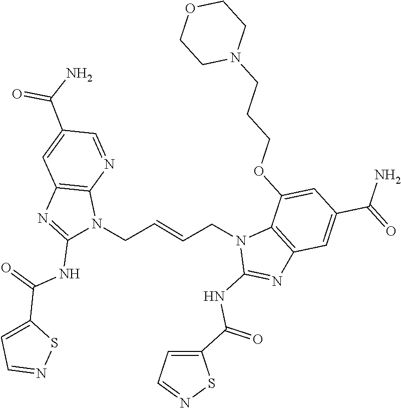 [SMILES: NC(=O)c1cnc2c(c1)nc(NC(=O)c1ccns1)n2C/C=C/Cn1c(NC(=O)c2ccns2)nc2cc(C(N)=O)cc(OCCCN3CCOCC3)c21] | Example 4 | 771 |
| 34 | 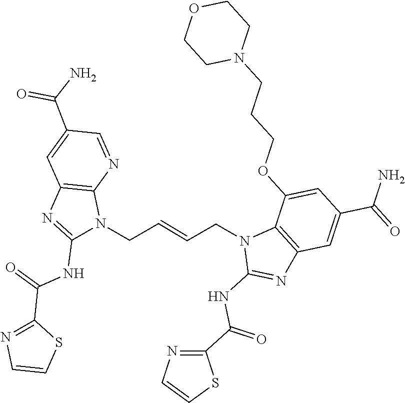 [SMILES: NC(=O)c1cnc2c(c1)nc(NC(=O)c1nccs1)n2C/C=C/Cn1c(NC(=O)c2nccs2)nc2cc(C(N)=O)cc(OCCCN3CCOCC3)c21] | Example 4 | 771 |
| 35 | 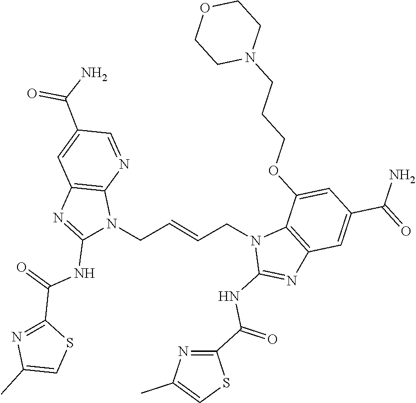 [SMILES: Cc1csc(C(=O)Nc2nc3cc(C(N)=O)cnc3n2C/C=C/Cn2c(NC(=O)c3nc(C)cs3)nc3cc(C(N)=O)cc(OCCCN4CCOCC4)c32)n1] | Example 4 | 799 |
| 36 | 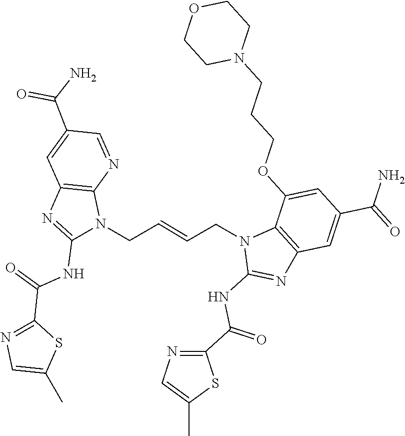 [SMILES: Cc1cnc(C(=O)Nc2nc3cc(C(N)=O)cnc3n2C/C=C/Cn2c(NC(=O)c3ncc(C)s3)nc3cc(C(N)=O)cc(OCCCN4CCOCC4)c32)s1] | Example 4 | 799 |
| 37 | 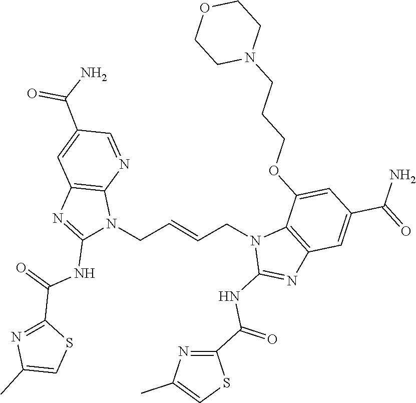 [SMILES: Cc1csc(C(=O)Nc2nc3cc(C(N)=O)cnc3n2C/C=C/Cn2c(NC(=O)c3nc(C)cs3)nc3cc(C(N)=O)cc(OCCCN4CCOCC4)c32)n1] | Example 4 | 799 |
| 38 | 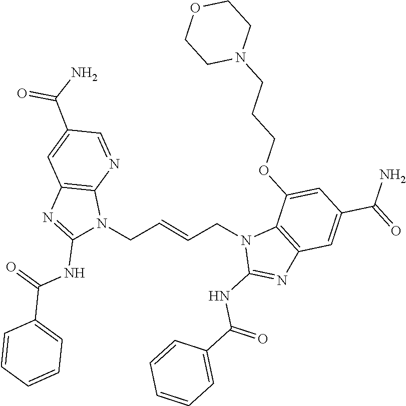 [SMILES: NC(=O)c1cnc2c(c1)nc(NC(=O)c1ccccc1)n2C/C=C/Cn1c(NC(=O)c2ccccc2)nc2cc(C(N)=O)cc(OCCCN3CCOCC3)c21] | Example 4 | 757 |
| 39 | 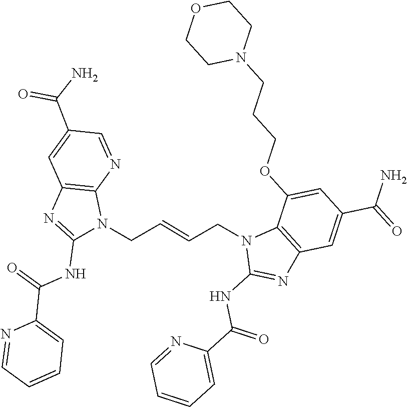 [SMILES: NC(=O)c1cnc2c(c1)nc(NC(=O)c1ccccn1)n2C/C=C/Cn1c(NC(=O)c2ccccn2)nc2cc(C(N)=O)cc(OCCCN3CCOCC3)c21] | Example 4 | 759 |
| 40 | 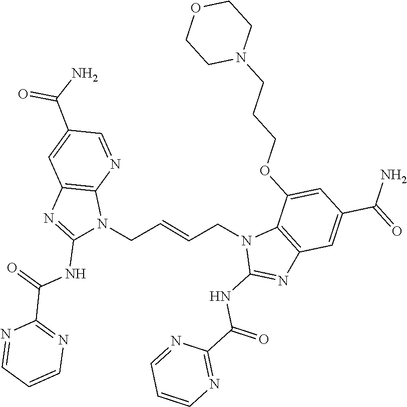 [SMILES: NC(=O)c1cnc2c(c1)nc(NC(=O)c1ncccn1)n2C/C=C/Cn1c(NC(=O)c2ncccn2)nc2cc(C(N)=O)cc(OCCCN3CCOCC3)c21] | Example 4 | 761 |
| 41 | 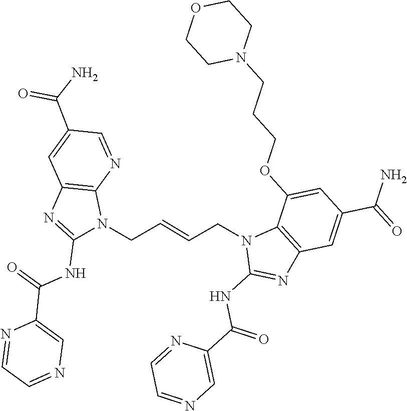 [SMILES: NC(=O)c1cnc2c(c1)nc(NC(=O)c1cnccn1)n2C/C=C/Cn1c(NC(=O)c2cnccn2)nc2cc(C(N)=O)cc(OCCCN3CCOCC3)c21] | Example 4 | 761 |
| 42 | 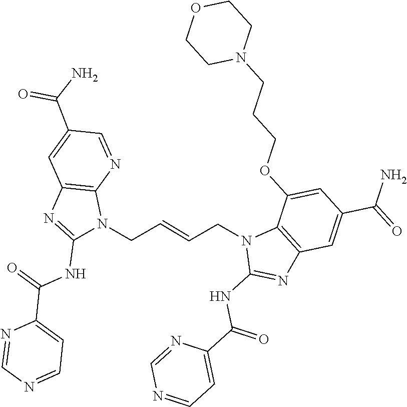 [SMILES: NC(=O)c1cnc2c(c1)nc(NC(=O)c1ccncn1)n2C/C=C/Cn1c(NC(=O)c2ccncn2)nc2cc(C(N)=O)cc(OCCCN3CCOCC3)c21] | Example 4 | 761 |
| 43 | 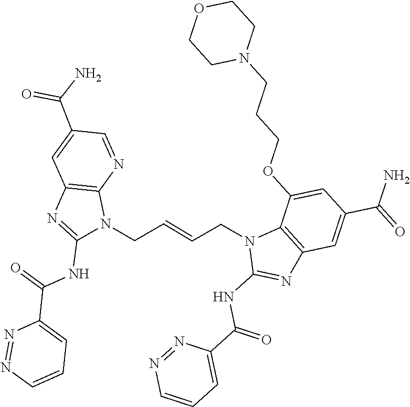 [SMILES: NC(=O)c1cnc2c(c1)nc(NC(=O)c1cccnn1)n2C/C=C/Cn1c(NC(=O)c2cccnn2)nc2cc(C(N)=O)cc(OCCCN3CCOCC3)c21] | Example 4 | 761 |
| 44 | 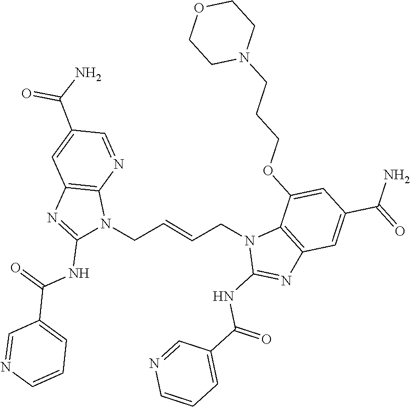 [SMILES: NC(=O)c1cnc2c(c1)nc(NC(=O)c1cccnc1)n2C/C=C/Cn1c(NC(=O)c2cccnc2)nc2cc(C(N)=O)cc(OCCCN3CCOCC3)c21] | Example 4 | 759 |
| 45 | 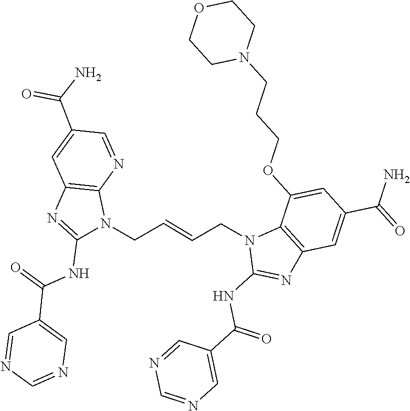 [SMILES: NC(=O)c1cnc2c(c1)nc(NC(=O)c1cncnc1)n2C/C=C/Cn1c(NC(=O)c2cncnc2)nc2cc(C(N)=O)cc(OCCCN3CCOCC3)c21] | Example 4 | 761 |
| 46 | 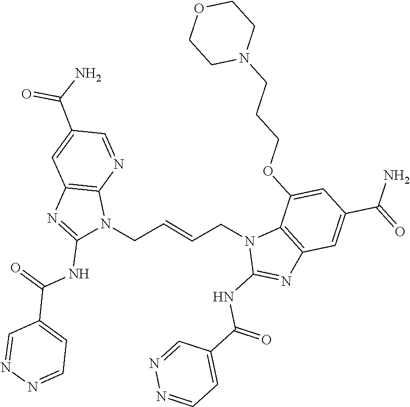 [SMILES: NC(=O)c1cnc2c(c1)nc(NC(=O)c1ccnnc1)n2C/C=C/Cn1c(NC(=O)c2ccnnc2)nc2cc(C(N)=O)cc(OCCCN3CCOCC3)c21] | Example 4 | 761 |

### Example 47

The Western blot analysis of activated STING and its downstream protein phosphorylation in human monocyte THP-1 cells

Human monocyte cell THP-1 were purchased from ATCC (Catalog No. TIB-202). The complete growth medium was made with ATCC-formulated RPMI-1640 Medium (Catalog No. 30-2001) and supplemented with 2-mercaptoethanol to a final concentration of 0.05 mM, and fetal bovine serum (FBS) to a final concentration of 10%.

After the THP-1 cells were treated with the 10 μM invented compounds for 3 hours, the cells were lysed by RIPA buffer, followed by washing with PBS. The total protein concentration was measured by BCA protein assay. The 10 μg protein sample was loaded after mixing with SDS-PAGE protein loading buffer, separated on SDS-PAGE and transferred to a PVDF membrane using the semi-dry transfer apparatus. After blocking with 5% BSA for 1 hr, the membrane was incubated with 1^{st} antibodies overnight at 4° C., followed by incubation with HRP-conjugated 2^{nd} antibodies (Catalog No. WBULS0100 from Millipore) for another 1 hr at room temperature. Lastly, the immunocomplexes were detected via the Bio-Rad ChemiDoc Touch Imaging System. Antibodies for western blotting were purchased from Cell Signaling Technology, catalog numbers 19781 (p-STING Ser366), 13647 (STING), 5483 (p-TBK1 Ser 172), 3504 (TBK1), 4947 (p-IRF3 Ser396), 4302 (IRF3) and 7074 (HRP-linked anti-rabbit IgG).

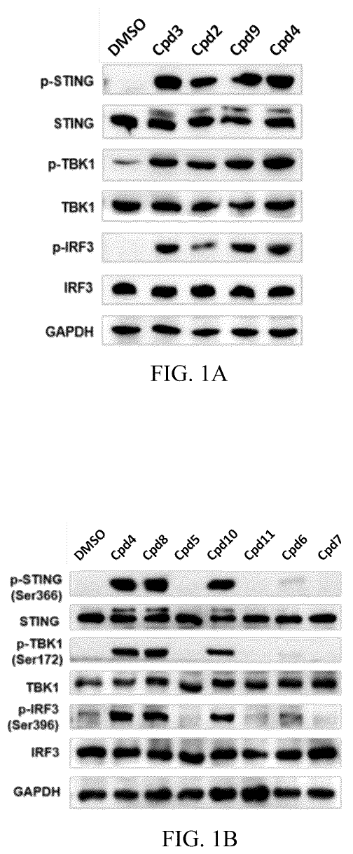

The western blot results are shown in FIGS. 1A and 1B. As demonstrated in FIG. 1A, all tested compounds significantly induced p-STING in THP-1 cells. Meanwhile, the active forms of its downstream targeting proteins TBK1 (p-TBK1) and IRF-3 (p-IRF-3) were also detected, indicating all tested compounds can activate the STING signal transduction pathway. In comparison, the STING pathway was inactivated without the STING agonist as indicated by lacking of its active form of phosphorylated STING (p-STING) in THP-1 cells when treated with DMSO. In FIG. 1B, only compounds 4, 8 and 10 remarkedly activated STING reflected by the higher levels of p-STING, p-TBK1 and p-IRF3 in compound-treated cells than DMSO-treated THP-1 cells at 10 μM for 3 hours. The phosphorylation activation was consistent with STING downstream targeting proteins TBK1 and IRF-3.

### Example 48

qPCR analysis of mRNA levels of cytokine gene expression in THP-1 cell Human monocyte cell THP-1 were purchased from ATCC (Catalog No. TIB-202). The complete growth medium was made with ATCC-formulated RPMI-1640 Medium (Catalog No. 30-2001) and supplemented with 2-mercaptoethanol to a final concentration of 0.05 mM, and fetal bovine serum (FBS) to a final concentration of 10%.

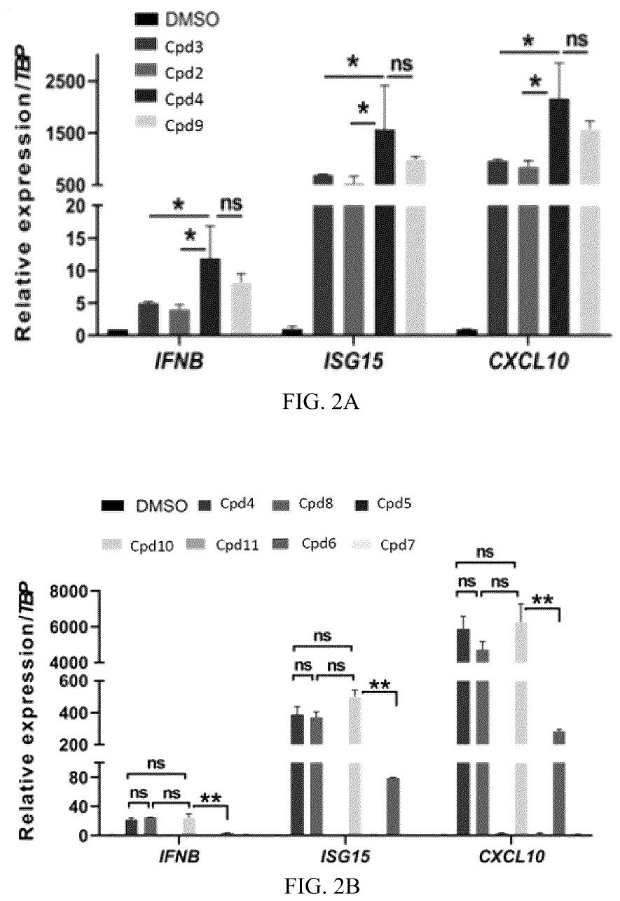

To evaluate the agonistic effects of chemicals on STING, the gene expression of IFNB, ISG15 and CXCL10 in human monocyte THP-1 cells was measured by real time qPCR. After the treatment of 10 μM Compounds for 6 hrs, cells were respectively lysed by TRIzol reagents (from Invitrogen). Total RNA was extracted according to the manufacturer's instruction. mRNA concentration was measured via 3rd generation NanoDrop machine. 1 μg of mRNA was used for the reverse transcription reaction with random primer, dNTP mix, RNase inhibitor and M-MuLV Reverse Transcriptase. SYBR green supermix and hot start DNA polymerase were used in qPCR with TBP gene as an internal control. Gene-specific primers were purchase from Integrated DNA Technologies. All samples were analyzed with Quant Studio 6 Flex from Applied Biosystems and the results are demonstrated in FIGS. 2A and 2B.

As shown in FIG. 2A, all four compounds significantly induce the gene expression of IFNB, ISG15 and CXCL10 after 6 hours of treatment in comparison with the control group in THP-1, suggesting that the activation of the STING pathway leads to the upregulation of its directly regulated target gene, interferon 0 (IFNB) and other cytokine genes ISG15 and CXCL10 expression. In FIG. 2B, the mRNA levels of IFNB, ISG15 and CXCL10 were significantly upregulated by Cpd4 (Example 4), Cpd6 (Example 6), Cpd8 (Example 8), Cpd10 (Example 10), after a 6-hour treatment compared with the DMSO group.

### Example 49

Evaluation of Antitumor Efficacy in CT-26 Mouse Model

Six- to eight-week-old male BALB/c mice of 18-20 g were purchased from Taconic Biosciences (La Jolla, CA). Mouse colon carcinoma CT-26 (from ATCC) cells were maintained in RPMI-1640 medium supplemented with 10% fetal bovine serum (FBS), 100 U/ml penicillin and 100 μg/ml streptomycin, and incubated at 37° C. and 5% CO₂. The cells growing in an exponential growth phase were harvested and counted for tumor inoculation. Each mouse was inoculated subcutaneously at the left and the right flank with 5×10⁴ CT-26 tumor cells in 0.1 ml of serum-free medium for tumor development.

Mice were randomized into study groups with 8 mice/group when average tumor volume was ˜70 mm³. On day 1, 4 and 8 mice were dosed with vehicle or 1.5 mg/kg Cpd 4 (Example 4) in 5% DMSO, 45% PEG400, and 55% saline formulation by intraperitoneal injection. Tumor size and body weight were measured twice per week throughout the study. Tumor volume was calculated by applying length and width caliper measurements to the following formula: TV=0.5*L*W². Percent treated/control (T/C) value was calculated as 100×ΔT/ΔC (when ΔT>0) or 100×ΔT/Tsd (when ΔT<0) where ΔT and ΔC are the changes in the mean tumor volumes between the indicated day and the first day of measurement for the treatment and control groups, respectively. Tsd is the first day mean tumor volume of the treated group. Statistical analyses of the differences in tumor volume among the groups were evaluated using a one-way ANOVA. All data were analyzed using GraphPad Prism 8 (GraphPad Software, San Diego, CA). p<0.05 was considered to be statistically significant.

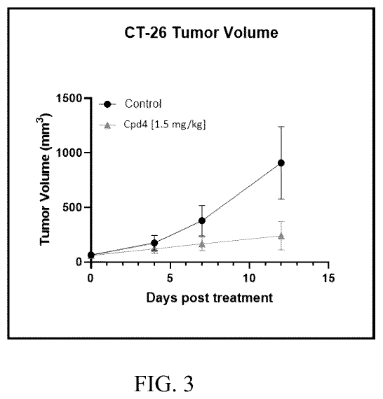

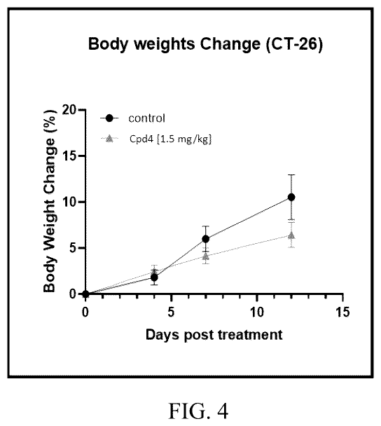

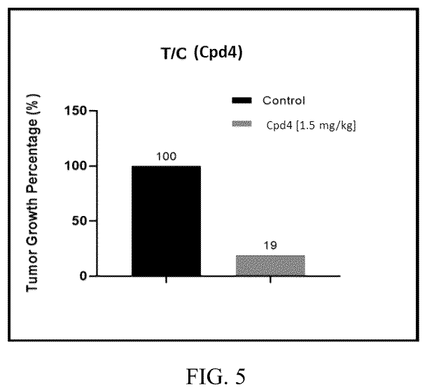

As shown in FIGS. 3-5, Cpd 4 (Example 4) is highly potent in the s.c. CT-26 mouse colon adenocarcinoma model in BALB/c mice, following 3×1.5 mg/kg doses of IP administrations. The tumor growth rate of treated/control (T/C, FIG. 5) was 19% on day 12. All mice were well-tolerated at 3×1.5 mg/kg dosage with no bodyweight loss (FIG. 4).

What is claimed is:

1. A compound having the Formula: 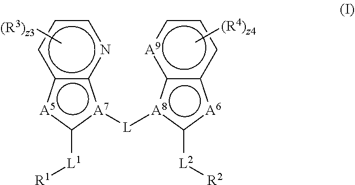
wherein:
R¹ is independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is independently halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O) NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, —O—P(O)(OH)₂, substituted or unsubstituted alkyl, substituted or unsubstituted heteroalkyl, or substituted or unsubstituted cycloalkyl;
z3 is 0, 1, 2, or 3;
X³ is independently —F, —Cl, —Br, or —I;
m3 and v3 are independently 1 or 2;
n3 is independently an integer from 0 to 4;
R^{3A}, R^{3B}, R^{3C}, and R^{3D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —O CI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl;
R⁴ is independently halogen, —CX⁴₃, —CHX⁴₂, —CH₂X⁴, —OCX⁴₃, —OCH₂X⁴, —OCHX⁴₂, —CN, —SO₄R^{4D}, —SOᵥ₄NR^{4A}R^{4B}, —N^{4C}NR^{4A}R^{4B}, —ONR^{4A}R^{4B}, —NHC(O)NR^{4C}NR^{4A}R^{4B}, —NHC(O)NR^{4A}R^{4B}, —N(O)ₘ₄, —NR^{4A}R^{4B}, C(O)R^{4C}, —C(O)OR^{4C}, —C(O) NR^{4A}R^{4B}, —ORD, —NR^{4A}SO₂R^{4D}, —NR^{4A}C(O)R^{4C}, —NR^{4A}C(O)OR^{4C}, —NR^{4A}OR^{4C}, —SF₅, —N₃, —O—P(O)(OH)₂, substituted or unsubstituted alkyl, substituted or unsubstituted heteroalkyl, or substituted or unsubstituted cycloalkyl;
z4 is 0, 1, 2, or 3;
X⁴ is independently —F, —Cl, —Br, or —I;
m4 and v4 are independently 1 or 2;
n4 is independently an integer from 0 to 4;
R^{4A}, R^{4B}, R^{4C}, and R^{4D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
L is -L³-L⁴-L⁵-;
L³ and L⁵ are independently a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is independently a bond, —N(R^{L4})—, —O—, —S—, —SO₂—, —C(O)—, —C(O)N(R^{L4})—, —N(R^{L4})C(O)—, —N(R^{L4})C(O)NH—, —NHC(O)N(R^{L4})—, —C(O)O—, —OC(O)—, —SO₂N(R^{L4})—, —N(R^{L4})SO₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R^{L4} is independently hydrogen, COR^{L4A}, CO₂H, CO₂R^{L4A}, SOR^{L4A}, SO₂R^{L4A}, CONH₂, CONR^{L4A}R^{L4B}, SO₂NH₂, SO₂NR^{L4A}R^{L4B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{L4A} and R^{L4B} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{L4A} and R^{L4B} substituents bonded to the same nitrogen atom may optionally be joined to form a substituted or unsubstituted heterocycloalkyl or substituted or unsubstituted heteroaryl;
L¹ is independently=NC(O)—, —C(O)N(R^{L1})—, or —N(R^{L1})C(O)—;
R^{L1} is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is independently=NC(O)—, —C(O)N(R^{L2})—, or —N(R^{L2})C(O)—;
R^{L2} is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
A⁵ is independently O, S, N, NR⁵, or CR⁵;
R⁵ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
A⁶ is independently O, S, N, NR⁶, or CR⁶;
R⁶ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
A⁷ is independently N or CR⁷;
R⁷ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
A⁸ is independently N or CR⁸;
R⁸ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
A⁹ is independently N or CR⁹;
R⁹ is independently hydrogen, halogen, —CX⁹₃, —CHX⁹₂, —CH₂X⁹, —OCX⁹₃, —OCH₂X⁹, —OCHX⁹₂, —CN, —SOₙ₉R^{9D}, —SOᵥ₉NR^{9A}R^{9B}, —NR^{9C}R^{9A}R^{9B}, —ONR^{9A}R^{9B}, —NHC(O)NR^{9C}NR^{9A}R^{9B}, —NHC(O)NR^{9A}R^{9B}, —N(O)ₘ₉, —NR^{9A}R^{9B}, —C(O)R^{9C}, —OC(O)R^{9C}, —C(O)—OR^{9C}, —OC(O)—OR^{9C}, —C(O)NR^{9A}R^{9B}, —OC(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, —SF₅, —N₃, O—P(O)(OR^{9A})₂, -(unsubstituted saturated alkyl)-O—P(O)(OR^{9A})₂, -(unsubstituted alkoxy)-O—P(O)(OR^{9A})₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z9 is 0, 1, 2, or 3;
X⁹ is independently —F, —Cl, —Br, or —I;
m9 and v9 are independently 1 or 2;
n9 is independently an integer from 0 to 4;
R^{9A}, R^{9B}, R^{9C}, and R^{9D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{9A} and R^{9B} substituents bonded to the same nitrogen atom may optionally be joined to form a substituted or unsubstituted heterocycloalkyl or substituted or unsubstituted heteroaryl;
or a tautomer thereof,
or a salt thereof.

2. The compound of claim 1, wherein
R¹ is independently R¹⁰-substituted or unsubstituted C₁-C₂₀ saturated alkyl, R¹⁰-substituted or unsubstituted C₂-C₂₀ alkenyl, R¹⁰-substituted or unsubstituted C₂-C₂₀ alkynyl, R¹⁰-substituted or unsubstituted C₃-C₁₀ cycloalkyl, R¹⁰-substituted or unsubstituted 4-10 membered heterocycloalkyl, R¹⁰-substituted or unsubstituted 6-10 membered aryl, or R¹⁰-substituted or unsubstituted 5-10 membered heteroaryl;
R¹⁰ is independently halogen, —CN, —CX¹⁰₃, —CHX¹⁰₂, —CH₂X¹⁰, —OR^{10B}, —OC(O)R^{10B}, —OC(O)NR^{10A}R^{10B}, —C(O)—OR^{10B}, —C(O)NR^{10A}R^{10B}, —SOR^{10B}, —SO₂R^{10B}, —SO₂NR^{10A}R^{10B}, —O—P(O)(OR_{10A})₂, —NR^{10A}R^{10B}, —NR^{10A}C(O)R^{10B}, —NR^{10A}SOR^{10B}, —NR^{10A}C(O)OR^{10B}, —NR^{10A}SO₂R^{10B}, —C(O)R^{10B}, -(unsubstituted alkyl)-O—P(O)(OR^{10A})₂, -(unsubstituted alkoxy)-O—P(O)(OR^{10A})₂, —OCX¹⁰₃, —OCH₂X¹⁰, —OCHX¹⁰₂, —SR^{10B}, —NHR^{10A}R^{10B}, —ONR^{10A}R^{10B}, —NHC(O)NHNR^{10A}R^{10B}, —NHC(O)NR^{10A}R^{10B}, —N(O)₂, —NR^{10A}OR^{10B}, —SF₅, —N₃, R¹¹-substituted or unsubstituted C₁-C₆ saturated alkyl, R¹¹-substituted or unsubstituted C₂-C₆ alkenyl, R¹¹-substituted or unsubstituted C₂-C₆ alkynyl, R¹¹-substituted or unsubstituted 2 to 8 membered heteroalkyl, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl;
X¹⁰ is independently —F, —Cl, —Br, or —I;
R^{10A} and R^{10B} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —COH, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R¹¹-substituted or unsubstituted C₁-C₁₀ saturated alkyl, R¹¹-substituted or unsubstituted C₂-C₁₀ alkenyl, R¹¹-substituted or unsubstituted C₂-C₁₀ alkynyl, R¹-substituted or unsubstituted 2 to 10 membered heteroalkyl, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl; R^{10A} and R^{10B} substituents bonded to the same nitrogen atom may optionally be joined to form a R¹¹-substituted or unsubstituted heterocycloalkyl or R¹¹-substituted or unsubstituted heteroaryl;
R¹¹ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, R¹²-substituted or unsubstituted C₁-C₄ saturated alkyl, R¹²-substituted or unsubstituted C₂-C₄ alkenyl, R¹²-substituted or unsubstituted C₂-C₄ alkynyl, R¹²-substituted or unsubstituted 2 to 12 membered heteroalkyl, R¹²-substituted or unsubstituted C₃-C₆ cycloalkyl, R¹²-substituted or unsubstituted 3 to 15 membered heterocycloalkyl, R¹²-substituted or unsubstituted phenyl, or R²-substituted or unsubstituted 5 to 6 membered heteroaryl;
R¹² is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, unsubstituted alkyl, unsubstituted heteroalkyl, unsubstituted cycloalkyl, unsubstituted heterocycloalkyl, unsubstituted aryl, or unsubstituted heteroaryl;
R² is independently R²⁰-substituted or unsubstituted C₁-C₂₀ saturated alkyl, R²⁰-substituted or unsubstituted C₂-C₂₀ alkenyl, R²⁰-substituted or unsubstituted C₂-C₂₀ alkynyl, R²⁰-substituted or unsubstituted C₃-C₁₀ cycloalkyl, R²⁰-substituted or unsubstituted 4-10 membered heterocycloalkyl, R²⁰-substituted or unsubstituted 6-10 membered aryl, or R²⁰-substituted or unsubstituted 5-10 membered heteroaryl;
R²⁰ is halogen, —CN, —CX²⁰₃, —CHX²⁰₂, —CH₂X²⁰, —OR^{20B}, —OC(O)R^{20B}, —OC(O)NR^{20A}R^{20B}, —C(O)—OR^{20B}, —C(O)NR^{20A}R^{20B}, —SOR^{20B}, —SO₂R^{20B}, —SO₂NR^{20A}R^{20B}, —O—P(O)(OR^{20A})₂, -(unsubstituted alkyl)-O—P(O)(OR^{20A})₂,-(unsubstituted alkoxy)-O—P(O)(OR^{20A})₂, —N_{20A}R^{20B}, NR^{20A}C(O)R^{20B}, —NR^{20A}SOR^{20B}, —NR^{20A}C(O)OR^{20B}, —N^{20A}SO₂R^{20B}, —C(O)R^{20B}, —OCX²⁰₃, —OCH₂X²⁰, —OCHX²⁰₂, —SR^{20B}, —NH^{20A}R^{20B}, —ONR^{20A}R^{20B}, —NHC(O)NHNR^{20A}R^{20B}, —NHC(O)NR^{20A}R^{20B}, —N(O)₂, —NR^{20A}OR^{20B}, —SF₅, —N₃, R²¹-substituted or unsubstituted C₁-C₆ saturated alkyl, R²¹-substituted or unsubstituted C₂-C₆ alkenyl, R²¹-substituted or unsubstituted C₂-C₆ alkynyl, R²¹-substituted or unsubstituted 2 to 8 membered heteroalkyl, R²¹-substituted or unsubstituted cycloalkyl, R²¹-substituted or unsubstituted heterocycloalkyl, R²¹-substituted or unsubstituted aryl, or R²¹-substituted or unsubstituted heteroaryl;
X²⁰ is independently —F, —Cl, —Br, or —I;
R^{20A} and R^{20B} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —COH, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R²¹-substituted or unsubstituted C₁-C₁₀ saturated alkyl, R²¹-substituted or unsubstituted C₂-C₁₀ alkenyl, R²¹-substituted or unsubstituted C₂-C₁₀ alkynyl, R²¹-substituted or unsubstituted 2 to 10 membered heteroalkyl, R²¹-substituted or unsubstituted cycloalkyl, R²¹-substituted or unsubstituted heterocycloalkyl, R²¹-substituted or unsubstituted aryl, or R²¹-substituted or unsubstituted heteroaryl; R^{20A} and R^{20B} substituents bonded to the same nitrogen atom may optionally be joined to form a R²¹-substituted or unsubstituted heterocycloalkyl or R²¹-substituted or unsubstituted heteroaryl;
R²¹ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, R²²-substituted or unsubstituted C₁-C₄ saturated alkyl, R²²-substituted or unsubstituted C₂-C₄ alkenyl, R²²-substituted or unsubstituted C₂-C₄ alkynyl, R²²-substituted or unsubstituted 2 to 12 membered heteroalkyl, R²²-substituted or unsubstituted C₃-C₆ cycloalkyl, R²²-substituted or unsubstituted 3 to 15 membered heterocycloalkyl, R²²-substituted or unsubstituted phenyl, or R²²-substituted or unsubstituted 5 to 6 membered heteroaryl;
R²² is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, unsubstituted alkyl, unsubstituted heteroalkyl, unsubstituted cycloalkyl, unsubstituted heterocycloalkyl, unsubstituted aryl, or unsubstituted heteroaryl;
R³ is independently halogen, —OR^{3D}, —CN, —CX³₃, —CHX³₂, —CH₂X³, —C(O)NR^{3A}R^{3B}, —C(O)—OR^{3C}, —O—P(O)(OH)₂, —NR^{3A}R^{3B}, —C(O)R^{3C}, —NR^{3A}C(O)R^{3C}, —NR^{3A}SO₂R^{3D}, —NR^{3C}SO₂(unsubstituted C₁-C₆ alkyl)-NR^{3A}R^{3B}, NR^{3C}C(O)(unsubstituted C₁-C₆alkyl)-NR^{3A}R^{3B}, (unsubstituted C₂-C₆alkyl)-NR^{3E}-(unsubstituted C₂-C₆ alkyl), R³¹-substituted or unsubstituted alkyl, R³¹-substituted or unsubstituted heteroalkyl, or R³¹-substituted or unsubstituted cycloalkyl;
R^{3A}, R^{3B}, R^{3C}, R^{3D}, R^{3F}, and R^{3G} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R³¹-substituted or unsubstituted alkyl, R³¹-substituted or unsubstituted heteroalkyl, R³¹-substituted or unsubstituted cycloalkyl, R³¹-substituted or unsubstituted heterocycloalkyl, R³¹-substituted or unsubstituted aryl, or R³¹-substituted or unsubstituted heteroaryl; R^{3A} and R^{3B} substituents bonded to the same nitrogen atom may optionally be joined to form a R³¹-substituted or unsubstituted heterocycloalkyl or R³¹-substituted or unsubstituted heteroaryl;
R^{3E} is independently hydrogen, —C(O)R^{3F}, —C(O)—OR^{3F}, —SOR^{3F}, —SO₂R^{3F}, —C(O)NR^{3F}R^{3G}, —SO₂NR^{3F}R^{3G}; —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R³¹-substituted or unsubstituted alkyl, R³¹-substituted or unsubstituted heteroalkyl, R³¹-substituted or unsubstituted cycloalkyl, R³¹-substituted or unsubstituted heterocycloalkyl, R³¹-substituted or unsubstituted aryl, or R³¹-substituted or unsubstituted heteroaryl; R^{3A} and R^{3B} substituents bonded to the same nitrogen atom may optionally be joined to form a R³¹-substituted or unsubstituted heterocycloalkyl or R³¹-substituted or unsubstituted heteroaryl;
R³¹ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, R³²-substituted or unsubstituted C₁-C₄ saturated alkyl, R³²-substituted or unsubstituted C₂-C₄ alkenyl, R³²-substituted or unsubstituted C₂-C₄ alkynyl, R³²-substituted or unsubstituted 2 to 12 membered heteroalkyl, R³²-substituted or unsubstituted C₃-C₆ cycloalkyl, R³²-substituted or unsubstituted 3 to 15 membered heterocycloalkyl, R³²-substituted or unsubstituted phenyl, or R³²-substituted or unsubstituted 5 to 6 membered heteroaryl;
R³² is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, R³³-substituted or unsubstituted C₁-C₄ saturated alkyl, R³³-substituted or unsubstituted C₂-C₄ alkenyl, R³³-substituted or unsubstituted C₂-C₄ alkynyl, R³³-substituted or unsubstituted 2 to 12 membered heteroalkyl, R³³-substituted or unsubstituted C₃-C₆ cycloalkyl, R³³-substituted or unsubstituted 3 to 15 membered heterocycloalkyl, R³³-substituted or unsubstituted phenyl, or R³³-substituted or unsubstituted 5 to 6 membered heteroaryl;
R³³ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, unsubstituted alkyl, unsubstituted heteroalkyl, unsubstituted cycloalkyl, unsubstituted heterocycloalkyl, unsubstituted aryl, or unsubstituted heteroaryl;
R⁴ is independently halogen, —OR^{4D}, —CN, —CX⁴₃, —CHX⁴₂, —CH₂X⁴, —C(O)NR^{4A}R^{4B}, —C(O)—OR^{4C}, —O—P(O)(OH)₂, —NR^{4A}R^{4B}, —C(O)R^{4C}, —NR^{4A}C(O)R^{4C}, —N^{4A}SO₂R^{4D}, —NR^{4C}SO₂(unsubstituted C₁-C₆ alkyl)-NR^{4A}R^{4B}, —NR^{4C}C(O)(unsubstituted C₁-C₆ alkyl)-NR^{4A}R^{4B}, (unsubstituted C₂-C₆ alkyl)-NR^{4E}-(unsubstituted C₂-C₆ alkyl), R⁴¹-substituted or unsubstituted alkyl, R⁴¹-substituted or unsubstituted heteroalkyl, or R⁴¹-substituted or unsubstituted cycloalkyl;
R^{4A}, R^{4B}, R^{4C}, R^{4D}, R^{4F}, and R^{4G} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R⁴¹-substituted or unsubstituted alkyl, R⁴¹-substituted or unsubstituted heteroalkyl, R⁴¹-substituted or unsubstituted cycloalkyl, R⁴¹-substituted or unsubstituted heterocycloalkyl, R⁴¹-substituted or unsubstituted aryl, or R⁴¹-substituted or unsubstituted heteroaryl; R^{4A} and R^{4B} substituents bonded to the same nitrogen atom may optionally be joined to form a R⁴¹-substituted or unsubstituted heterocycloalkyl or R⁴¹-substituted or unsubstituted heteroaryl; R^{4F} and R substituents bonded to the same nitrogen atom may optionally be joined to form a R⁴¹-substituted or unsubstituted heterocycloalkyl or R⁴¹-substituted or unsubstituted heteroaryl;
R^{4E} is independently hydrogen, —C(O)R^{4F}, —C(O)—OR^{4F}, —SOR^{4F}, —SO₂R^{4F}, —C(O)NR^{4F}R^{4G}, —SO₂NR^{4F}R^{4G}; —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R⁴¹-substituted or unsubstituted alkyl, R⁴¹-substituted or unsubstituted heteroalkyl, R⁴¹-substituted or unsubstituted cycloalkyl, R⁴¹-substituted or unsubstituted heterocycloalkyl, R⁴¹-substituted or unsubstituted aryl, or R⁴¹-substituted or unsubstituted heteroaryl;
R⁴¹ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, R⁴²-substituted or unsubstituted C₁-C₄ saturated alkyl, R⁴²-substituted or unsubstituted C₂-C₄ alkenyl, R⁴²-substituted or unsubstituted C₂-C₄ alkynyl, R⁴²-substituted or unsubstituted 2 to 12 membered heteroalkyl, R⁴²-substituted or unsubstituted C₃-C₆ cycloalkyl, R⁴²-substituted or unsubstituted 3 to 15 membered heterocycloalkyl, R⁴²-substituted or unsubstituted phenyl, or R⁴²-substituted or unsubstituted 5 to 6 membered heteroaryl;
R⁴² is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, R⁴³-substituted or unsubstituted C₁-C₄ saturated alkyl, R⁴³-substituted or unsubstituted C₂-C₄ alkenyl, R⁴³-substituted or unsubstituted C₂-C₄ alkynyl, R⁴³-substituted or unsubstituted 2 to 12 membered heteroalkyl, R⁴³-substituted or unsubstituted C₃-C₆ cycloalkyl, R⁴³-substituted or unsubstituted 3 to 15 membered heterocycloalkyl, R⁴³-substituted or unsubstituted phenyl, or R⁴³-substituted or unsubstituted 5 to 6 membered heteroaryl;
R⁴³ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, unsubstituted alkyl, unsubstituted heteroalkyl, unsubstituted cycloalkyl, unsubstituted heterocycloalkyl, unsubstituted aryl, or unsubstituted heteroaryl;
L is R^{L}-substituted or unsubstituted C₁-C₁₀ saturated alkyl, R^{L}-substituted or unsubstituted C₂-C₁₀ alkenyl, R^{L}-substituted or unsubstituted C₂-C₁₀ alkynyl, R^{L}-substituted or unsubstituted C₂-C₆ alkyl-O—C₂-C₆ alkyl, R^{L}-substituted or unsubstituted C₂ alkyl-O—C₂ alkyl, R^{L}-substituted or unsubstituted C₂ alkyl-O—C₂ alkyl-O—C₂ alkyl, R^{L}-substituted or unsubstituted C₂-C₆ alkyl-N(R^{L4})—C₂-C₆ alkyl, R^{L}-substituted or unsubstituted C₃-C₆ cycloalkyl, R^{L}-substituted or unsubstituted phenyl, R^{L}-substituted or unsubstituted 4-6 membered heterocycloalkyl, R^{L}-substituted or unsubstituted 5-6 membered heteroaryl, R^{L}-substituted or unsubstituted C₁-C₄alkyl-(C₃-C₆ cycloalkyl)-C₁-C₄alkyl, R^{L}-substituted or unsubstituted C₁-C₄ alkyl-phenyl-C₁-C₄ alkyl, R^{L}-substituted or unsubstituted C₁-C₄ alkyl-(4-6 membered heterocycloalkyl)-C₁-C₄ alkyl, or R^{L}-substituted or unsubstituted C₁-C₄ alkyl-(5-6 membered heteroaryl)-C₁-C₄ alkyl;
R^{L} is halogen, —OR^{LB}, —O—P(O)(OR^{LA})₂, -(unsubstituted alkyl)-O—P(O)(OR^{LA})₂, -(unsubstituted alkoxy)-O—P(O)(OR^{LA})₂, —NR^{LA}R^{LB}, —OC(O)R^{LB}, —C(O)—OR^{LB}, —SOR^{LB}, —SO₂R^{LB}, —C(O)NR^{LA}R^{LB}, —SO₂NR^{LA}R^{LB}, —OC(O)NR^{LA}R^{LB}, —NR^{LA}C(O)R^{LB}, —NR^{LA}SOR^{LB}, —NR^{LA}C(O)OR^{LB}, —NR^{LA}SO₂R^{LB}, —R^{LA} SO₂R^{LB}, —CN, —CX^{L}₃, —CHX^{L}₂, —CH₂X^{L}, —C(O)R^{LB}, —OCX^{L}₃, —OCH₂X^{L}, —OCHX^{L}₂, —SR^{LB}, —NHNR^{LA}R^{LB}, —ONR^{LA}R^{LB}, —NHC(O)NHNR^{LA}R^{LB}, —NHC(O)NR^{LA}R^{LB}, —N(O)₂, —NR^{LA}OR^{LB}, —SF₅, —N₃, R^{L6}-substituted or unsubstituted C₁-C₆ saturated alkyl, R^{L6}-substituted or unsubstituted C₂-C₆ alkenyl, R^{L6}-substituted or unsubstituted C₂-C₆ alkynyl, R^{L6}-substituted or unsubstituted 2 to 12 membered heteroalkyl, R^{L6}-substituted or unsubstituted cycloalkyl, R^{L6}-substituted or unsubstituted heterocycloalkyl, R^{L6}-substituted or unsubstituted aryl, or R^{L6}-substituted or unsubstituted heteroaryl;
X^{L} is independently —F, —Cl, —Br, or —I;
R^{LA} and R^{LB} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —COH, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R^{L6}-substituted or unsubstituted saturated alkyl, R^{L6}-substituted or unsubstituted alkenyl, R^{L6}-substituted or unsubstituted alkynyl, R^{L6}-substituted or unsubstituted heteroalkyl, R^{L6}-substituted or unsubstituted cycloalkyl, R^{L6}-substituted or unsubstituted heterocycloalkyl, R^{L6}-substituted or unsubstituted aryl, or R^{L6}-substituted or unsubstituted heteroaryl;
R^{L6} is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, R^{L7}-substituted or unsubstituted C₁-C₄ saturated alkyl, R^{L7}-substituted or unsubstituted C₂-C₄ alkenyl, R^{L7}-substituted or unsubstituted C₂-C₄ alkynyl, R^{L7}-substituted or unsubstituted 2 to 12 membered heteroalkyl, R^{L7}-substituted or unsubstituted C₃-C₆ cycloalkyl, R^{L7}-substituted or unsubstituted 3 to 15 membered heterocycloalkyl, R^{L7}-substituted or unsubstituted phenyl, or R^{L7}-substituted or unsubstituted 5 to 6 membered heteroaryl;
R^{L7} is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, unsubstituted alkyl, unsubstituted heteroalkyl, unsubstituted cycloalkyl, unsubstituted heterocycloalkyl, unsubstituted aryl, or unsubstituted heteroaryl;
R^{L4} is independently hydrogen, COR^{L4A}, CO₂H, CO₂R^{L4A}, SOR^{L4A}, SO₂R^{L4A}, CONH₂, CONR^{L4A}R^{L4B}, SO₂NH₂, or SO₂NR^{L4A}R^{LB}; R^{L6}-substituted or unsubstituted alkyl, R^{L6}-substituted or unsubstituted heteroalkyl, R^{L6}-substituted or unsubstituted cycloalkyl, R^{L6}-substituted or unsubstituted heterocycloalkyl, R^{L6}-substituted or unsubstituted aryl, or R^{L6}-substituted or unsubstituted heteroaryl;
R^{L4A} and R^{L4B} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R^{L6}-substituted or unsubstituted alkyl, R^{L6}-substituted or unsubstituted heteroalkyl, R^{L6}-substituted or unsubstituted cycloalkyl, R^{L6}-substituted or unsubstituted heterocycloalkyl, R^{L6}-substituted or unsubstituted aryl, or R^{L6}-substituted or unsubstituted heteroaryl; R^{L4A} and R^{L4B} substituents bonded to the same nitrogen atom may optionally be joined to form a R^{L6}-substituted or unsubstituted heterocycloalkyl or R^{L6}-substituted or unsubstituted heteroaryl;
L¹ is independently=NC(O)—, —C(O)N(R^{L1})—, or —N(R^{L1})C(O)—;
R^{L1} is independently hydrogen, unsubstituted C₁-C₆ saturated alkyl, unsubstituted C₂-C₆ alkenyl, unsubstituted C₂-C₆ alkynyl, unsubstituted 2 to 6 membered heteroalkyl, unsubstituted C₃-C₆ cycloalkyl, or unsubstituted 3 to 6 membered heterocycloalkyl;
L² is independently=NC(O)—, —C(O)N(R^{L2})—, or —N(R^{L2})C(O)—;
R^{L2} is independently hydrogen, unsubstituted C₁-C₆ saturated alkyl, unsubstituted C₂-C₆ alkenyl, unsubstituted C₂-C₆ alkynyl, unsubstituted 2 to 6 membered heteroalkyl, unsubstituted C₃-C₆ cycloalkyl, or unsubstituted 3 to 6 membered heterocycloalkyl;
A⁵ is independently O, S, N, NR⁵, or CR⁵;
R⁵ is independently hydrogen, unsubstituted C₁-C₆ saturated alkyl, unsubstituted C₂-C₆ alkenyl, unsubstituted C₂-C₆ alkynyl, unsubstituted 2 to 6 membered heteroalkyl, unsubstituted C₃-C₆ cycloalkyl, or unsubstituted 3 to 6 membered heterocycloalkyl;
A⁶ is independently O, S, N, NR⁶, or CR⁶;
R⁶ is independently hydrogen, unsubstituted C₁-C₆ saturated alkyl, unsubstituted C₂-C₆ alkenyl, unsubstituted C₂-C₆ alkynyl, unsubstituted 2 to 6 membered heteroalkyl, unsubstituted C₃-C₆ cycloalkyl, or unsubstituted 3 to 6 membered heterocycloalkyl;
A⁷ is independently N or CR⁷;
R⁷ is independently hydrogen, unsubstituted C₁-C₆ saturated alkyl, unsubstituted C₂-C₆ alkenyl, unsubstituted C₂-C₆ alkynyl, unsubstituted 2 to 6 membered heteroalkyl, unsubstituted C₃-C₆ cycloalkyl, or unsubstituted 3 to 6 membered heterocycloalkyl;
A⁸ is independently N or CR⁸;
R⁸ is independently hydrogen, unsubstituted C₁-C₆ saturated alkyl, unsubstituted C₂-C₆ alkenyl, unsubstituted C₂-C₆ alkynyl, unsubstituted 2 to 6 membered heteroalkyl, unsubstituted C₃-C₆ cycloalkyl, or unsubstituted 3 to 6 membered heterocycloalkyl;
A⁹ is independently N or CR⁹;
R⁹ is independently hydrogen, halogen, —CN, —CX⁹₃, —CHX⁹₂, —CH₂X⁹, O—P(O)(OR^{9A})₂, -(unsubstituted saturated alkyl)-O—P(O)(OR^{9A})₂, -(unsubstituted alkoxy)-O—P(O)(OR^{9A})₂, —OR^{9D}, —NR^{9B}R^{9B}, —NR^{9A}R^{9B}, —OC(O)NR^{9A}R^{9B}, —OC(O)R^{9C}, —C(O)R^{9C}, —C(O)—OR^{9C}, —C(O)NR^{9A}R^{9B}, —SOR^{9D}, —SO₂NR^{9A}R^{9B}, —SO₂R^{9D}, —NR^{9A}SO₂R^{9D}, —NR^{9A}C(O)R^{9C}, —NR^{9C}SO₂(u nsubstituted C₁-C₄ alkyl)-NR^{9A}R^{9B}, —N(R^{9C})CO(unsubstituted C₁-C₄ alkyl)-^{9A}R^{9B}, —O(unsubstituted C₁-C₄ alkyl)-NR^{9A}R^{9B}, —O(unsubstituted C₁-C₄ alkyl)-OR^{9D}, —O(unsubstituted C₁-C₄ alkyl)-OR^{9B}, —NR^{9C}(unsubstituted C₁-C₄ alkyl)NR^{9A}R^{9B}, —OCX⁹₃, —OCH₂X⁹, —OCHX⁹₂, —NR^{9C}NR^{9A}R^{9B}, —NR^{9A}R^{9B}, —NHC(O)NR^{9C}NR^{9A}R^{9B}, —NHC(O)NR^{9A}R^{9B}, —N(O)ₘ₉, —NR^{9A}C(O)OR^{9C}, —NR^{9A}OR^{9C}, —SF₅, —N₃, R⁹⁰-substituted or unsubstituted alkyl, R⁹⁰-substituted or unsubstituted heteroalkyl, R⁹⁰-substituted or unsubstituted cycloalkyl, R⁹⁰-substituted or unsubstituted heterocycloalkyl, R⁹⁰-substituted or unsubstituted aryl, or R⁹⁰-substituted or unsubstituted heteroaryl;
X⁹ is independently —F, —Cl, —Br, or —I;
R^{9A}, R^{9B}, R^{9C}, and R^{9D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R⁹⁰-substituted or unsubstituted saturated alkyl (e.g., C₁-C₈, C₁-C₆, C₁-C₄, or C₁-C₂), R⁹⁰-substituted or unsubstituted alkenyl (e.g., C₂-C₈, C₂-C₆, C₂-C₄, or C₂), R⁹⁰-substituted or unsubstituted alkynyl (e.g., C₂-C₈, C₂-C₆, C₂-C₄, or C₂), R⁹⁰-substituted or unsubstituted heteroalkyl (e.g., 2 to 8 membered, 2 to 6 membered, 4 to 6 membered, 2 to 3 membered, or 4 to 5 membered), R⁹⁰-substituted or unsubstituted cycloalkyl (e.g., C₃-C₈, C₃-C₆, C₄-C₆, or C₅-C₆), R⁹⁰-substituted or unsubstituted heterocycloalkyl (e.g., 3 to 8 membered, 3 to 6 membered, 4 to 6 membered, 4 to 5 membered, or 5 to 6 membered), R⁹⁰-substituted or unsubstituted aryl (e.g., C₆-C₁₀ or phenyl), or R⁹⁰-substituted or unsubstituted heteroaryl (e.g., 5 to 10 membered, 5 to 9 membered, or 5 to 6 membered); R^{9A} and R^{9B} substituents bonded to the same nitrogen atom may optionally be joined to form a R⁹⁰-substituted or unsubstituted heterocycloalkyl (e.g., 3 to 8 membered, 3 to 6 membered, 4 to 6 membered, 4 to 5 membered, or 5 to 6 membered) or R⁹⁰-substituted or unsubstituted heteroaryl (e.g., 5 to 10 membered, 5 to 9 membered, or 5 to 6 membered);
R⁹⁰ is halogen, —OR^{90B}, —O—P(O)(OR^{90A})₂, —NR^{90A}R^{90B}, —NR^{90B}R^{90B}, —C(O)—OR^{90B}, —C(O)NR^{90A}R^{90B}, —SOR^{90B}, —SO₂R^{90B}, —SO₂NR^{90A}R^{90B}, —OC(O)R^{90B}, —OC(O)NR^{90A}R^{90B}, —NR^{90A}C(O)R^{90B}, —NR^{90A}SOR^{90B}, —NR^{90A}C(O)OR^{90B}, —C(O)R^{90B}, —CN, —CX⁹₃, —CHX⁹⁰₂, —CH₂X⁹⁰, —NR^{90A}SO₂R^{90B}, —OCX⁹⁰₃, —OCH₂X⁹⁰, —OCHX⁹⁰₂, —SR^{90B}, NHNR^{90A}R^{90B}, —ONR^{90A}R^{90B}, —NHC(O)NHNR^{90A}R^{90B}, —NHC(O)NR^{90A}R^{90B}, —N(O)₂, —NR^{90A}OR^{90B}, —SF₅, —N₃, -(unsubstituted saturated alkyl)-O—P(O)(OR^{91A})₂, -(unsubstituted alkoxy)-O— P(O)(OR^{90A})₂, R⁹¹-substituted or unsubstituted C₁-C₆ saturated alkyl, R⁹¹-substituted or unsubstituted C₂-C₆ alkenyl, R⁹¹-substituted or unsubstituted C₂-C₆ alkynyl, R⁹¹-substituted or unsubstituted 2 to 12 membered heteroalkyl, R⁹¹-substituted or unsubstituted cycloalkyl, R⁹¹-substituted or unsubstituted heterocycloalkyl, R⁹¹-substituted or unsubstituted aryl, or R⁹¹-substituted or unsubstituted heteroaryl;
X⁹⁰ is independently —F, —Cl, —Br, or —I;
R^{90A} and R^{90B} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —COH, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, R⁹¹-substituted or unsubstituted saturated alkyl, R⁹¹-substituted or unsubstituted alkenyl, R⁹¹-substituted or unsubstituted alkynyl, R⁹¹-substituted or unsubstituted heteroalkyl, R⁹¹-substituted or unsubstituted cycloalkyl, R⁹¹-substituted or unsubstituted heterocycloalkyl, R⁹¹-substituted or unsubstituted aryl, or R⁹¹-substituted or unsubstituted heteroaryl; R^{90A} and R^{90B} substituents bonded to the same nitrogen atom may optionally be joined to form a R⁹¹-substituted or unsubstituted heterocycloalkyl or R⁹¹-substituted or unsubstituted heteroaryl;
R⁹¹ is independently oxo, halogen, —CCl₃, —CBr₃, —CF₃, —CI₃, —CHCl₂, —CHBr₂, —CHF₂, —CHI₂, -CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, R⁹²-substituted or unsubstituted C₁-C₄ saturated alkyl, R⁹²-substituted or unsubstituted C₂-C₄ alkenyl, R⁹²-substituted or unsubstituted C₂-C₄ alkynyl, R⁹²-substituted or unsubstituted 2 to 12 membered heteroalkyl, R⁹²-substituted or unsubstituted C₃-C₆ cycloalkyl, R⁹²-substituted or unsubstituted 3 to 15 membered heterocycloalkyl, R⁹²-substituted or unsubstituted phenyl, or R⁹²-substituted or unsubstituted 5 to 6 membered heteroaryl;
R⁹² is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COH, —COOH, —CONH₂, —OCOH, —OCOOH, —OCONH₂, —NO₂, —SH, —SO₃H, —SO₄H, —SO₂NH₂, —NHSOH, —NHSO₂H, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, —O—P(O)(OH)₂, unsubstituted alkyl, unsubstituted heteroalkyl, unsubstituted cycloalkyl, unsubstituted heterocycloalkyl, unsubstituted aryl, or unsubstituted heteroaryl;
or a tautomer thereof,
or a salt thereof.

3. The compound of claim 1, wherein
R¹ is independently R¹⁰-substituted or unsubstituted C₁-C₂₀ saturated alkyl, R¹⁰-substituted or unsubstituted C₂-C₂₀ alkenyl, R¹⁰-substituted or unsubstituted C₂-C₂₀ alkynyl, R¹⁰-substituted or unsubstituted C₃-C₁₀ cycloalkyl, R¹⁰-substituted or unsubstituted phenyl, R¹⁰-substituted or unsubstituted 6-10 membered aryl, R¹⁰-substituted or unsubstituted 4-10 membered heterocycloalkyl, or R¹⁰-substituted or unsubstituted 5-10 membered heteroaryl;
wherein said R¹⁰-substituted C₁-C₂₀ saturated alkyl, R¹⁰-substituted C₂-C₂₀ alkenyl, R¹⁰-substituted C₂-C₂₀ alkynyl, R¹⁰-substituted C₃-C₁₀ cycloalkyl, R¹⁰-substituted phenyl, R¹⁰-substituted 6-10 membered aryl, R¹⁰-substituted 4-10 membered heterocycloalkyl, and R¹⁰-substituted 5-10 membered heteroaryl is substituted with 1 to 4 R¹⁰ substituents;
each R¹⁰ substituent is independently halogen, —CN, —CF₃, —OR^{10B}, —OC(O)R^{10B}, —OC(O)NR^{10A}R^{10B}, —C(O)—OR^{10B}, —C(O)NR^{10A}R^{10B}, —SOR^{10B}, —SO₂R^{10B}, —SO₂NR^{10A}R^{10B}, —O—P(O)(OR^{10A})₂, —NR^{10A}R^{10B}, —NR^{10A}C(O)R^{10B}, —NR^{10A}SOR^{10B}, —NR^{10A}C(O)OR^{10B}, —NR^{10A}SO₂R^{10B}, —C(O)R^{10B}, unsubstituted C₁-C₆ saturated alkyl, unsubstituted C₂-C₆ alkenyl, unsubstituted C₂-C₆alkynyl, halo-substituted (C₁-C₄ alkyl), hydroxy-substituted (C₁-C₄ alkyl), (unsubstituted C₁-C₄ alkyl)-O—P(O)(OH)₂, (unsubstituted C₁-C₄ alkyl)-O—P(O)(OR^{10A})₂, halo-substituted (C₁-C₄ alkoxy), unsubstituted C₁-C₆ alkylthio, hydroxy-substituted (C₂-C₄ alkoxy), (unsubstituted C₂-C₄ alkoxy)-O—P(O)(OH)₂, (unsubstituted C₂-C₄ alkoxy)-O—P(O)(OR^{10A})₂, unsubstituted C₁-C₄ alkoxy(unsubstituted C₁-C₄ alkoxy);
R^{10A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy
R^{10B} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R¹¹-substituted or unsubstituted phenyl, R¹¹-substituted or unsubstituted heteroaryl, R¹¹-substituted or unsubstituted heterocycloalkyl, or R¹¹-substituted or unsubstituted heteroaryl; wherein said R¹¹-substituted or unsubstituted phenyl, R¹¹-substituted or unsubstituted heteroaryl, R¹¹-substituted or unsubstituted heterocycloalkyl, and R¹¹-substituted or unsubstituted heteroaryl are optionally substituted with one or more R¹¹ substituents;
R¹¹ is independently halogen, CN, CF₃, unsubstituted C₁-C₄ saturated alkyl, halo-substituted (saturated C₁-C₄ alkyl), unsubstituted C₂-C₄ alkenyl, unsubstituted C₂-C₄ alkynyl, or unsubstituted C₃-C₆ cycloalkyl;
R² is independently R²⁰-substituted or unsubstituted C₁-C₂₀ saturated alkyl, R²⁰-substituted or unsubstituted C₂-C₂₀ alkenyl, R²⁰-substituted or unsubstituted C₂-C₂₀ alkynyl, R²⁰-substituted or unsubstituted C₃-C₁₀ cycloalkyl, R²⁰-substituted or unsubstituted phenyl, R²⁰-substituted or unsubstituted 6-10 membered aryl, R²⁰-substituted or unsubstituted 4-10 membered heterocycloalkyl, or R²⁰-substituted or unsubstituted 5-10 membered heteroaryl;
wherein said R²⁰-substituted C₁-C₂₀ saturated alkyl, R²⁰-substituted C₂-C₂₀ alkenyl, R²⁰-substituted C₂-C₂₀ alkynyl, R²⁰-substituted C₃-C₁₀ cycloalkyl, R²⁰-substituted phenyl, R²⁰-substituted 6-10 membered aryl, R²⁰-substituted 4-10 membered heterocycloalkyl, and R²⁰-substituted 5-10 membered heteroaryl is substituted with 1 to 4 R²⁰ substituents;
each R²⁰ substituent is independently halogen, —CN, —CF₃, —OR^{20B}, —OC(O)R^{20B}, —OC(O)NR^{20A}R^{20B}, —C(O)—OR^{20B}, —C(O)NR^{20A}R^{20B}, —SOR^{20B}, —SO₂R^{20B}, —SO₂NR^{20A}R^{20B}, —O—P(O)(OR^{20A})₂, —N^{20A}R^{20B}, —NR^{20A}C(O)R^{20B}, —NR^{20A}SOR^{20B}, —NR^{20A}C(O)OR^{20B}, —N^{20A}SO₂R^{20B}, —C(O)R^{20B}, unsubstituted C₁-C₆ saturated alkyl, unsubstituted C₂-C₆ alkenyl, unsubstituted C₂-C₆ alkynyl, halo-substituted (C₁-C₄ alkyl), hydroxy-substituted (C₁-C₄ alkyl), (unsubstituted C₁-C₄ alkyl)-O—P(O)(OH)₂, (unsubstituted C₁-C₄ alkyl)-O—P(O)OR^{20A})₂, halo-substituted (C₁-C₄ alkoxy), unsubstituted C₁-C₆ alkylthio, hydroxy-substituted (C₂-C₄ alkoxy), (unsubstituted C₂-C₄ alkoxy)-O—P(O)(OH)₂, (unsubstituted C₂-C₄ alkoxy)-O—P(O)(OR^{20A})₂, unsubstituted C₁-C₄ alkoxy(unsubstituted C₁-C₄ alkoxy);
R^{20A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy
R^{20B} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R²¹-substituted or unsubstituted phenyl, R²¹-substituted or unsubstituted heteroaryl, R²¹-substituted or unsubstituted heterocycloalkyl, or R²¹-substituted or unsubstituted heteroaryl; wherein said R²¹-substituted or unsubstituted phenyl, R²¹-substituted or unsubstituted heteroaryl, R²¹-substituted or unsubstituted heterocycloalkyl, and R²¹-substituted or unsubstituted heteroaryl are optionally substituted with one or more R^{2′} substituents;
R²¹ is independently halogen, CN, CF₃, unsubstituted C₁-C₄ saturated alkyl, halo-substituted (saturated C₁-C₄ alkyl), unsubstituted C₂-C₄ alkenyl, unsubstituted C₂-C₄ alkynyl, or unsubstituted C₃-C₆ cycloalkyl;
R³ is independently halogen, —OH, —CN, —CF₃, —C(O)NH₂, —C(O)OR^{3J}, —O—P(O)(OH)₂, —NR^{3A}R^{3B}, —C(O)R^{3C}, —NR^{3A}C(O)R^{3C}, —NR^{3A}SO₂R^{3D}, unsubstituted C₂-C₆ saturated alkyl-NR^{3E}— unsubstituted C₂-C₆ saturated alkyl, N(R^{3H})SO₂(unsubstituted C₁-C₆ saturated alkyl)-N(R^{3H})(R^{3I}), N(R^{3H})CO(unsubstituted C₁-C₆ saturated alkyl)-N(R^{3H}(R^{3I}), unsubstituted C₁-C₆ saturated alkyl, unsubstituted C₂-C₆ alkenyl, unsubstituted C₂-C₆ alkynyl, unsubstituted C₁-C₆ saturated alkylthio, or unsubstituted C₁-C₆ cycloalkyl;
R^{3A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy;
R^{3B}, R^{3C}, and R^{3D} are independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R³¹-substituted or unsubstituted phenyl, R³¹-substituted or unsubstituted heteroaryl, R³¹-substituted or unsubstituted heterocycloalkyl, or R³-substituted or unsubstituted heteroaryl; wherein said R³¹-substituted or unsubstituted phenyl, R³¹-substituted or unsubstituted heteroaryl, R³¹-substituted or unsubstituted heterocycloalkyl, and R³¹-substituted or unsubstituted heteroaryl are optionally substituted with one or more R³¹ substituents;
R³¹ is independently halogen, CN, CF₃, unsubstituted C₁-C₄ saturated alkyl, halo-substituted (saturated C₁-C₄ alkyl), unsubstituted C₂-C₄ alkenyl, unsubstituted C₂-C₄ alkynyl, or unsubstituted C₃-C₆ cycloalkyl;
R^{3J} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, substituted or unsubstituted aryl, substituted or unsubstituted heteroaryl, or substituted or unsubstituted heterocycloalkyl, wherein said substituted aryl, substituted heteroaryl, and substituted heterocyclic groups are independently substituted with one or more halogen, CN, CF₃, or unsubstituted C₁-C₄ saturated alkyl;
R^{3E} is independently hydrogen, —C(O)R^{3F}, —C(O)OR^{3F}, —SOR^{3F}, —SO₂R^{3F}, —C(O)NR^{3F}R^{3G}, or —SO₂NR^{3F}R^{3G};
R^{3G} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy;
R^{3F} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R³¹-substituted or unsubstituted phenyl, R³¹-substituted or unsubstituted heteroaryl, R³¹-substituted or unsubstituted heterocycloalkyl, or R³¹-substituted or unsubstituted heteroaryl; wherein said R³¹-substituted or unsubstituted phenyl, R³¹-substituted or unsubstituted heteroaryl, R³¹-substituted or unsubstituted heterocycloalkyl, and R³¹-substituted or unsubstituted heteroaryl are optionally substituted with one or more R³¹ substituents;
R^{3H} and R^{3I} are independently hydrogen, substituted or unsubstituted C₁-C₆ saturated alkyl, wherein said substituted saturated alkyl is independently substituted with one or more halogen, OH, amine, CN, CF₃ or unsubstituted C₁-C₄ saturated alkyl; or R^{3H} and R^{3I}, taken together with the atom or atoms through which they are connected to form a substituted or unsubstituted 5-6 membered heterocycloalkyl or substituted or unsubstituted 5-6 membered heteroaryl, wherein the substituted 5-6 membered heterocycloalkyl or substituted 5-6 membered heteroaryl is independently substituted with one or more halogen, OH, amine, CN, CF₃, or unsubstituted C₁-C₄ saturated alkyl, wherein said amine substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy;
R⁴ is independently halogen, —OH, —CN, —CF₃, —C(O)NH₂, —C(O)OR^{4J}, —O—P(O)(OH)₂, —N_{4A}R^{4B}, —C(O)R^{4C}, —NR^{4A}C(O)R^{4C}, —NR^{4A}SO₂R^{4D}, unsubstituted C₂-C₆ saturated alkyl-N^{4E}— unsubstituted C₂-C₆ saturated alkyl, N(R^{4H})SO₂(unsubstituted C₁-C₆ saturated alkyl)-N(R^{4H})(R^{4I}), N(R^{4H})CO(unsubstituted C₁-C₆ saturated alkyl)-N(R^{4H}) R^{4I}), unsubstituted C₁-C₆ saturated alkyl, unsubstituted C₂-C₆ alkenyl, unsubstituted C₂-C₆ alkynyl, unsubstituted C₁-C₆ saturated alkylthio, or unsubstituted C₁-C₆ cycloalkyl;
R^{4A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy
R^{4B}, R^{4C}, and R^{4D} are independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R⁴¹-substituted or unsubstituted phenyl, R⁴¹-substituted or unsubstituted heteroaryl, R⁴¹-substituted or unsubstituted heterocycloalkyl, or R⁴¹-substituted or unsubstituted heteroaryl; wherein said R⁴¹-substituted or unsubstituted phenyl, R⁴¹-substituted or unsubstituted heteroaryl, R⁴¹-substituted or unsubstituted heterocycloalkyl, and R⁴¹-substituted or unsubstituted heteroaryl are optionally substituted with one or more R⁴¹ substituents;
R⁴¹ is independently halogen, CN, CF₃, unsubstituted C₁-C₄ saturated alkyl, halo-substituted (saturated C₁-C₄ alkyl), unsubstituted C₂-C₄ alkenyl, unsubstituted C₂-C₄ alkynyl, or unsubstituted C₃-C₆ cycloalkyl;
R^{4J} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, substituted or unsubstituted aryl, substituted or unsubstituted heteroaryl, or substituted or unsubstituted heterocycloalkyl, wherein said substituted aryl, substituted heteroaryl, and substituted heterocyclic groups are independently substituted with one or more halogen, CN, CF₃, or unsubstituted C₁-C₄ saturated alkyl;
R^{4E} is independently hydrogen, —C(O)R^{4F}, —C(O)OR^{4F}, —SOR^{4F}, —SO₂R^{4F}, —C(O)NR^{4F}R^{4G}, or —SO₂NR^{4F}R^{4G};
R^{4G} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy;
R^{4F} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R⁴¹-substituted or unsubstituted phenyl, R⁴¹-substituted or unsubstituted heteroaryl, R⁴¹-substituted or unsubstituted heterocycloalkyl, or R⁴¹-substituted or unsubstituted heteroaryl; wherein said R⁴¹-substituted or unsubstituted phenyl, R⁴¹-substituted or unsubstituted heteroaryl, R¹-substituted or unsubstituted heterocycloalkyl, and R⁴¹-substituted or unsubstituted heteroaryl are optionally substituted with one or more R⁴¹ substituents;
R^{4H} and R^{4I} are independently hydrogen, substituted or unsubstituted C₁-C₆ saturated alkyl, wherein said substituted saturated alkyl is independently substituted with one or more halogen, OH, amine, CN, CF₃, or unsubstituted C₁-C₄ saturated alkyl; or R^{4H} and R^{4I}, taken together with the atom or atoms through which they are connected to form a substituted or unsubstituted 5-6 membered heterocycloalkyl or substituted or unsubstituted 5-6 membered heteroaryl, wherein the substituted 5-6 membered heterocycloalkyl or substituted 5-6 membered heteroaryl is independently substituted with one or more halogen, OH, amine, CN, CF₃, or unsubstituted C₁-C₄ saturated alkyl, wherein said amine substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy;
L is halo-substituted (C₁-C₁₀ saturated alkylene), R^{L}-substituted or unsubstituted C₁-C₁₀ alkylene, R^{L}-substituted or unsubstituted C₂-C₁₀ alkenylene, R^{L}-substituted or unsubstituted C₂-C₁₀ alkynylene, R^{L}-substituted or unsubstituted C₂-C₆ saturated alkylene-O—C₂—C₆ saturated alkylene, R^{L}-substituted or unsubstituted C₂ saturated alkylene-O—C₂ saturated alkylene, R^{L}-substituted or unsubstituted C₂ saturated alkylene-O—C₂ saturated alkylene-O—C₂ saturated alkylene, R^{L}-substituted or unsubstituted C₂-C₆ saturated alkylene-NR^{L4}—C₂-C₆ saturated alkylene, R^{L}-substituted or unsubstituted C₃-C₆ cycloalkylene, R^{L}-substituted or unsubstituted phenylene, R^{L}-substituted or unsubstituted 4-6 membered heterocycloalkylene, R^{L}-substituted or unsubstituted 5-6 membered heteroarylene, R^{L}-substituted or unsubstituted C₁-C₄ saturated alkylene-(C₃-C₆ cycloalkylene)-C₁-C₄ saturated alkylene, R^{L}-substituted or unsubstituted C₁-C₄ saturated alkylene-phenylene-C₁-C₄ saturated alkylene, R^{L}-substituted or unsubstituted C₁-C₄ saturated alkylene-(4-6 membered heterocycloalkylene)-C₁-C₄ saturated alkylene, or R^{L}-substituted or unsubstituted C₁-C₄ saturated alkylene-(5-6 membered heteroarylene)-C₁-C₄ saturated alkylene;
wherein said R^{L}-substituted C₁-C₁₀ alkylene, R^{L}-substituted C₂-C₁₀ alkenylene, R^{L}-substituted C₂-C₁₀ alkynylene, R^{L}-substituted C₂-C₆ saturated alkylene-O—C₂-C₆ saturated alkylene, R^{L}-substituted C₂ saturated alkylene-O—C₂ saturated alkylene, R^{L}-substituted C₂ saturated alkylene-O—C₂ saturated alkylene-O—C₂ saturated alkylene, R^{L}-substituted C₂-C₆ saturated alkylene-NR⁴—C₂-C₆ saturated alkylene, R^{L}-substituted C₃-C₆ cycloalkylene, R^{L}-substituted phenylene, R^{L}-substituted 4-6 membered heterocycloalkylene, R^{L}-substituted 5-6 membered heteroarylene, R^{L}-substituted C₁-C₄ saturated alkylene-(C₃-C₆ cycloalkylene)-C₁-C₄ saturated alkylene, R^{L}-substituted C₁-C₄ saturated alkylene-phenylene-C₁-C₄ saturated alkylene, R^{L}-substituted C₁-C₄ saturated alkylene-(4-6 membered heterocycloalkylene)-C₁-C₄ saturated alkylene, and R^{L}-substituted C₁-C₄ saturated alkylene-(5-6 membered heteroarylene)-C₁-C₄ saturated alkylene are independently substituted with 1 or 2 R^{L};
R^{L} is independently halogen, —OR^{LB}, —O—P(O)(OR^{LA})₂, —NR^{LA}R^{LB}, —OC(O)R^{LB}, —C(O)—OR^{LB}, —SOR^{LB}, —SO₂R^{LB}, —C(O)NR^{LA}R^{LB}, —SO₂NR^{L A}R^{LB}, —OC(O)NR^{LA}R^{LB}, —NR^{LA}C(O)R^{LB}, —NR^{LA}SOR^{LB}, —NR^{LA}C(O)OR^{LB}, —NR^{LA}SO₂R^{LB}, —R^{LA}S O₂R^{LB}, halo-substituted (saturated C₁-C₄alkyl), (unsubstituted C₁-C₄ saturated alkyl)(unsubstituted C₁-C₄ saturated alkyl)amino, unsubstituted C₁-C₄ saturated alkyl, hydroxy-substituted (C₁-C₄ saturated alkyl), (unsubstituted C₁-C₄ saturated alkyl)-O—P(O)(OH)₂, (unsubstituted C₁-C₄ saturated alkyl)-O—P(O)OR^{LA})₂, halo-substituted (C₁-C₄ alkoxy), unsubstituted C₁-C₄ alkoxy, unsubstituted C₁-C₆ saturated alkylthio, hydroxy-substituted (C₂-C₄ alkoxy), (unsubstituted C₂-C₄ alkoxy)-O—P(O)(OH)₂, (unsubstituted C₂-C₄alkoxy)-O—P(O)(OR^{LA})₂, unsubstituted C₁-C₄ alkoxy(unsubstituted C₁-C₄ alkoxy), COR^{LA}, or CON(R^{LA})(R^{LC});
R^{LA} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy
R^{LB} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R^{L6}-substituted or unsubstituted phenyl, R^{L6}-substituted or unsubstituted heteroaryl, R^{L6}-substituted or unsubstituted heterocycloalkyl, or R^{L6}-substituted or unsubstituted heteroaryl; wherein said R^{L6}-substituted or unsubstituted phenyl, R^{L6}-substituted or unsubstituted heteroaryl, R^{L6}-substituted or unsubstituted heterocycloalkyl, and R^{L6}-substituted or unsubstituted heteroaryl are optionally substituted with one or more R^{L6} substituents;
R^{L6} is independently halogen, CN, CF₃, unsubstituted C₁-C₄ saturated alkyl, halo-substituted (saturated C₁-C₄ alkyl), unsubstituted C₂-C₄ alkenyl, unsubstituted C₂-C₄ alkynyl, or unsubstituted C₃-C₆ cycloalkyl;
R^{LC} is independently hydrogen, substituted or unsubstituted C₁-C₆ saturated alkyl, wherein said substituted saturated alkyl is independently substituted with one or more halogen, OH, amine, CN, CF₃, or unsubstituted C₁-C₄ saturated alkyl;
R^{L4} is independently hydrogen, COR^{L4A}, CO₂H, CO₂R^{L4A}, SOR^{L4A}, SO₂R^{L4A}, CONH₂, CONR^{L4A}R^{L4B}, SO₂NH₂, or SO₂NR^{L4A}R^{L4B};
R^{L4B} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy;
R^{L4A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R^{L6}-substituted or unsubstituted phenyl, R^{L6}-substituted or unsubstituted heterocycloalkyl, or R^{L6}-substituted or unsubstituted heteroaryl; wherein said R^{L6}-substituted or unsubstituted phenyl, R^{L6}-substituted or unsubstituted heterocycloalkyl, and R^{L6}-substituted or unsubstituted heteroaryl are optionally substituted with one or more R^{L6} substituents;
L¹ is independently=NC(O)—, —C(O)N(R^{L1})—, or —N(R^{L1})C(O)—;
R^{L1} is independently hydrogen, unsubstituted C₁-C₆ saturated alkyl, unsubstituted C₂-C₆ alkenyl, unsubstituted C₂-C₆ alkynyl, or unsubstituted C₃-C₆ cycloalkyl;
L² is independently=NC(O)—, —C(O)N(R^{L2})—, or —N(R^{L2})C(O)—;
R^{L2} is independently hydrogen, unsubstituted C₁-C₆ saturated alkyl, unsubstituted C₂-C₆ alkenyl, unsubstituted C₂-C₆ alkynyl, or unsubstituted C₃-C₆ cycloalkyl;
A⁵ is independently O, S, N, NR⁵, or CR⁵;
R⁵ is independently hydrogen, unsubstituted C₁-C₆ saturated alkyl, unsubstituted C₂-C₆ alkenyl, unsubstituted C₂-C₆ alkynyl, or unsubstituted C₃-C₆ cycloalkyl;
A⁶ is independently O, S, N, NR⁶, or CR⁶;
R⁶ is independently hydrogen, unsubstituted C₁-C₆ saturated alkyl, unsubstituted C₂-C₆ alkenyl, unsubstituted C₂-C₆ alkynyl, or unsubstituted C₃-C₆ cycloalkyl;
A⁷ is independently N or CR⁷;
R⁷ is independently hydrogen, unsubstituted C₁-C₆ saturated alkyl, unsubstituted C₂-C₆ alkenyl, unsubstituted C₂-C₆ alkynyl, or unsubstituted C₃-C₆ cycloalkyl;
A⁸ is independently N or CR⁸;
R⁸ is independently hydrogen, unsubstituted C₁-C₆ saturated alkyl, unsubstituted C₂-C₆ alkenyl, unsubstituted C₂-C₆ alkynyl, or unsubstituted C₃-C₆ cycloalkyl;
A⁹ is independently N or CR⁹;
R⁹ is independently hydrogen, halogen, —CN, —CF₃, O—P(O)(OR^{9A})₂, —OR^{9D}, —NR^{9B}R^{9B}, —NR^{9A}R^{9B}, —OC(O)NR^{9A}R^{9B}, —OC(O)R^{9C}, —C(O)R^{9C}, —C(O)—OR^{9J}, —C(O)NR^{9A}R^{9B}, —SOR^{9D}, —SO₂NR^{9A}R^{9B}, —SO₂R^{9J}, —NR^{9A}SO₂R^{9D}, —NR^{9A}C(O)R^{9C}, N(R^{9H})SO₂(unsubstituted C₁-C₄ saturated alkyl)-N(R^{9I})(R^{9H}), N(R^{9H})CO(unsubstituted C₁-C₄ saturated alkyl)-N(R^{9I}(R^{9H}), O(unsubstituted C₁-C₄ saturated alkyl)-N(R^{9I})(R^{9H}), O(unsubstituted C₁-C₄ saturated alkyl)-OR^{9B}, NR^{9H}(unsubstituted C₁-C₄ saturated alkyl)-N(R^{9I})(R^{9H}), halo-substituted (C₁-C₁₀ saturated alkyl), halo-substituted (C₁-C₁₀ alkoxy), R⁹⁰-substituted or unsubstituted (C₁-C₁₀ saturated alkyl)oxy, R⁹⁰-substituted or unsubstituted (C₁-C₁₀ saturated alkyl)amino, R⁹⁰-substituted or unsubstituted (C₁-C₆ saturated alkyl)(C₁-C₄ saturated alkyl)amino, R⁹⁰-substituted or unsubstituted phenyl, R⁹⁰-substituted or unsubstituted 5-6 membered heterocycloalkyl, or R⁹⁰-substituted or unsubstituted 5-6 membered heteroaryl;
R^{9A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy
R^{9B}, R^{9C}, and R^{9D} are independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R⁹¹-substituted or unsubstituted phenyl, R⁹¹-substituted or unsubstituted heteroaryl, R⁹¹-substituted or unsubstituted heterocycloalkyl, or R⁹¹-substituted or unsubstituted heteroaryl; wherein said R⁹¹-substituted or unsubstituted phenyl, R⁹¹-substituted or unsubstituted heteroaryl, R⁹¹-substituted or unsubstituted heterocycloalkyl, and R⁹¹-substituted or unsubstituted heteroaryl are optionally substituted with one or more R⁹¹ substituents;
R⁹¹ is independently halogen, CN, CF₃, unsubstituted C₁-C₄ saturated alkyl, halo-substituted (saturated C₁-C₄ alkyl), unsubstituted C₂-C₄ alkenyl, unsubstituted C₂-C₄ alkynyl, or unsubstituted C₃-C₆ cycloalkyl;
R^{9J} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, substituted or unsubstituted aryl, substituted or unsubstituted heteroaryl, or substituted or unsubstituted heterocycloalkyl, wherein said substituted aryl, substituted heteroaryl, and substituted heterocyclic groups are independently substituted with one or more halogen, CN, CF₃, or unsubstituted C₁-C₄ saturated alkyl;
R^{9H} and R^{9I} are independently hydrogen, substituted or unsubstituted C₁-C₆ saturated alkyl, wherein said substituted saturated alkyl is independently substituted with one or more halogen, OH, amine, CN, CF₃, or unsubstituted C₁-C₄ saturated alkyl; or R⁹H and R^{9I}, taken together with the atom or atoms through which they are connected to form a substituted or unsubstituted 5-6 membered heterocycloalkyl or substituted or unsubstituted 5-6 membered heteroaryl, wherein the substituted 5-6 membered heterocycloalkyl or substituted 5-6 membered heteroaryl is independently substituted with one or more halogen, OH, amine, CN, CF₃, or unsubstituted C₁-C₄ saturated alkyl, wherein said amine substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy;
wherein said R⁹⁰-substituted (C₁-C₁₀ saturated alkyl)oxy, R⁹⁰-substituted (C₁-C₁₀ saturated alkyl)amino, R⁹⁰-substituted (C₁-C₆ saturated alkyl)(C₁-C₄ saturated alkyl)amino, R⁹⁰-substituted phenyl, R⁹⁰-substituted 5-6 membered heterocycloalkyl, or R⁹⁰-substituted 5-6 membered heteroaryl is substituted with 1-4 independent R⁹⁰ and
R⁹⁰ is halogen, —OR^{90B}, —O—P(O)(OR^{90A})₂, —NR^{90A}R^{90B}, —NR^{90B}R^{90B}, —C(O)—OR^{90B}, —C(O)NR^{90A}R^{90B}, —SOR^{90B}, —SO₂R^{90B}, —SO₂NR^{90A}R^{90B}, —OC(O)R^{90B}, —OC(O)NR^{90A}R^{90B}, —NR^{90A}C(O)R^{90B}, —NR^{90A}SOR^{90B}, —NR^{90A}C(O)OR^{90B}, —C(O)R^{90B}, —SO₂R^{90J}, —C(O)—OR^{90J}, —C(O)NR^{90A}R^{90H}, —C(O)R^{90A}, (unsubstituted C₁-C₄ saturated alkyl)(unsubstituted C₁-C₄ saturated alkyl)amino, unsubstituted C₁-C₄ saturated alkyl, halo-substituted (C₁-C₄ saturated alkyl), hydroxy-substituted (C₁-C₄ saturated alkyl), (unsubstituted C₁-C₄ saturated alkyl)-O—P(O)(OH)₂, (unsubstituted C₁-C₄ saturated alkyl)-O—P(O)(OR^{90A})₂, halo-substituted (C₁-C₄ alkoxy), unsubstituted C₁-C₄ alkoxy, hydroxy-substituted (C₂-C₄ alkoxy), (unsubstituted C₂-C₄ alkoxy)-O—P(O)(OH)₂, (unsubstituted C₂-C₄alkoxy)-O—P(O)(OR^{90A})₂, or unsubstituted C₁-C₄ alkoxy-(unsubstituted C₁-C₄ alkoxy);
R^{90A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy
R^{9B} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R⁹¹-substituted or unsubstituted phenyl, R⁹¹-substituted or unsubstituted heteroaryl, R⁹¹-substituted or unsubstituted heterocycloalkyl, or R⁹¹-substituted or unsubstituted heteroaryl; wherein said R⁹¹-substituted or unsubstituted phenyl, R⁹¹-substituted or unsubstituted heteroaryl, R⁹¹-substituted or unsubstituted heterocycloalkyl, and R⁹¹-substituted or unsubstituted heteroaryl are optionally substituted with one or more R⁹¹ substituents;
R⁹¹ is independently halogen, CN, CF₃, unsubstituted C₁-C₄ saturated alkyl, halo-substituted (saturated C₁-C₄ alkyl), unsubstituted C₂-C₄ alkenyl, unsubstituted C₂-C₄ alkynyl, or unsubstituted C₃-C₆ cycloalkyl;
R^{90J} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, substituted or unsubstituted aryl, substituted or unsubstituted heteroaryl, or substituted or unsubstituted heterocycloalkyl, wherein said substituted aryl, substituted heteroaryl, and substituted heterocyclic groups are independently substituted with one or more halogen, CN, CF₃, or unsubstituted C₁-C₄ saturated alkyl;
R^{90H} is independently hydrogen, substituted or unsubstituted C₁-C₆ saturated alkyl, wherein said substituted saturated alkyl is independently substituted with one or more halogen, OH, amine, CN, CF₃, or unsubstituted C₁-C₄ saturated alkyl, wherein said amine substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy;
or a tautomer thereof,
or a salt thereof.

4. A compound having formula (Ia): 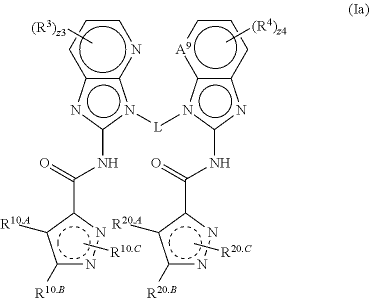
wherein
R^{10.A} is independently hydrogen, halogen, or unsubstituted C₁-C₄ saturated alkyl;
R^{10.B} is independently hydrogen, R¹-substituted or unsubstituted C₁-C₆ saturated alkyl, R¹¹-substituted or unsubstituted C₂-C₆ alkenyl, R¹-substituted or unsubstituted C₂-C₆ alkynyl, R¹¹-substituted or unsubstituted 2 to 7 membered alkoxy, R¹-substituted or unsubstituted C₁-C₆ alkylamino, R¹¹-substituted or unsubstituted C₃-C₆ cycloalkyl, R¹-substituted or unsubstituted aryl, R¹¹-substituted or unsubstituted heterocycloalkyl, or R″-substituted or unsubstituted heteroaryl;
R^{10.C} is independently hydrogen, R¹¹-substituted or unsubstituted C₁-C₆ saturated alkyl, R¹¹-substituted or unsubstituted C₂-C₆ alkenyl, R¹¹-substituted or unsubstituted C₂-C₆ alkynyl, or R¹¹-substituted or unsubstituted C₃-C₆ cycloalkyl;
R¹¹ is independently halogen, —CN, —CF₃, —OR^{11B}, —NR^{11A}R^{11B}, —C(O)—OR^{11B}, —C(O)NR^{11A}R^{11B}, —SO₂NR^{11A}R^{11B}, or —OC(O)NR^{11A}R^{11B};
R^{11A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy;
R^{11B}, is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R¹²-substituted or unsubstituted phenyl, R¹²-substituted or unsubstituted heteroaryl, R¹² -substituted or unsubstituted heterocycloalkyl, or R¹²-substituted or unsubstituted heteroaryl; wherein said R¹²-substituted or unsubstituted phenyl, R¹²-substituted or unsubstituted heteroaryl, R¹²-substituted or unsubstituted heterocycloalkyl, and R¹²-substituted or unsubstituted heteroaryl are optionally substituted with one or more R¹² substituents;
R¹² is independently halogen, CN, CF₃, unsubstituted C₁-C₄ saturated alkyl, halo-substituted (saturated C₁-C₄ alkyl), unsubstituted C₂-C₄ alkenyl, unsubstituted C₂-C₄ alkynyl, or unsubstituted C₃-C₆ cycloalkyl;
R^{20.A} is independently hydrogen, halogen, or unsubstituted C₁-C₄ saturated alkyl;
R^{20.B} is independently hydrogen, R²¹-substituted or unsubstituted C₁-C₆ saturated alkyl, R²¹-substituted or unsubstituted C₂-C₆ alkenyl, R²¹-substituted or unsubstituted C₂-C₆ alkynyl, R²¹-substituted or unsubstituted 2 to 7 membered alkoxy, R²¹-substituted or unsubstituted C₁-C₆ alkylamino, R²¹-substituted or unsubstituted C₃-C₆ cycloalkyl, R²¹-substituted or unsubstituted aryl, R²¹-substituted or unsubstituted heterocycloalkyl, or R²¹-substituted or unsubstituted heteroaryl;
R^{20.C} is independently hydrogen, R²¹-substituted or unsubstituted C₁-C₆ saturated alkyl, R²¹-substituted or unsubstituted C₂-C₆ alkenyl, R²¹-substituted or unsubstituted C₂-C₆ alkynyl, or R²¹-substituted or unsubstituted C₃-C₆ cycloalkyl;
R²¹ is independently halogen, —CN, —CF₃, —OR^{21B}, —NR^{21A}R^{21B}, —C(O)—OR²¹B, —C(O)NR^{21A}R^{21B}, —SO₂NR^{21A}R^{21B}, or —OC(O)NR^{21A}R^{21B};
R^{21A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy;
R^{21B}, is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R²²-substituted or unsubstituted phenyl, R²²-substituted or unsubstituted heteroaryl, R²²-substituted or unsubstituted heterocycloalkyl, or R²²-substituted or unsubstituted heteroaryl; wherein said R²²-substituted or unsubstituted phenyl, R²²-substituted or unsubstituted heteroaryl, R²²-substituted or unsubstituted heterocycloalkyl, and R²²-substituted or unsubstituted heteroaryl are optionally substituted with one or more R²² substituents;
R²² is independently halogen, CN, CF₃, unsubstituted C₁-C₄ saturated alkyl, halo-substituted (saturated C₁-C₄ alkyl), unsubstituted C₂-C₄ alkenyl, unsubstituted C₂-C₄ alkynyl, or unsubstituted C₃-C₆ cycloalkyl;
R³ is independently halogen, —OH, —CN, —CF₃, —C(O)NH₂, —C(O)OR^{3J}, —O—P(O)(OH)₂, —NR^{3A}R^{3B}, —C(O)R^{3C}, —NR^{3A}C(O)R^{3C}, —NR^{3A}SO₂R^{3D}, unsubstituted C₂-C₆ saturated alkyl-NR^{3E}— unsubstituted C₂-C₆ saturated alkyl, N(R^{3H})SO₂(unsubstituted C₁-C₆ saturated alkyl)-N(R^{3H})(R^{3I}), N(R^{3H})CO(unsubstituted C₁-C₆ saturated alkyl)-N(R^{3H}(R^{3I}), unsubstituted C₁-C₆ saturated alkyl, unsubstituted C₂-C₆ alkenyl, unsubstituted C₂-C₆ alkynyl, unsubstituted C₁-C₆ saturated alkylthio, or unsubstituted C₁-C₆ cycloalkyl;
R^{3A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy;
R^{3B}, R^{3C}, and R^{3D} are independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R³¹-substituted or unsubstituted phenyl, R³¹-substituted or unsubstituted heteroaryl, R³¹-substituted or unsubstituted heterocycloalkyl, or R³¹-substituted or unsubstituted heteroaryl; wherein said R³¹-substituted or unsubstituted phenyl, R³¹-substituted or unsubstituted heteroaryl, R³¹-substituted or unsubstituted heterocycloalkyl, and R³¹-substituted or unsubstituted heteroaryl are optionally substituted with one or more R^{3′} substituents;
R³¹ is independently halogen, CN, CF₃, unsubstituted C₁-C₄ saturated alkyl, halo-substituted (saturated C₁-C₄ alkyl), unsubstituted C₂-C₄ alkenyl, unsubstituted C₂-C₄ alkynyl, or unsubstituted C₃-C₆ cycloalkyl;
R^{3J} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, substituted or unsubstituted aryl, substituted or unsubstituted heteroaryl, or substituted or unsubstituted heterocycloalkyl, wherein said substituted aryl, substituted heteroaryl, and substituted heterocyclic groups are independently substituted with one or more halogen, CN, CF₃, or unsubstituted C₁-C₄ saturated alkyl;
R^{3E} is independently hydrogen, —C(O)R^{3F}, —C(O)OR^{3F}, —SOR^{3F}, —SO₂R^{3F}, —C(O)NR^{3F}R^{3G}, or —SO₂NR^{3F}R^{3G};
R^{3G} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy;
R^{3F} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R³¹-substituted or unsubstituted phenyl, R³¹-substituted or unsubstituted heteroaryl, R³¹-substituted or unsubstituted heterocycloalkyl, or R³¹-substituted or unsubstituted heteroaryl; wherein said R³¹-substituted or unsubstituted phenyl, R³¹-substituted or unsubstituted heteroaryl, R³¹-substituted or unsubstituted heterocycloalkyl, and R³¹-substituted or unsubstituted heteroaryl are optionally substituted with one or more R³¹ substituents;
R^{3H} and R^{3I} are independently hydrogen, substituted or unsubstituted C₁-C₆ saturated alkyl, wherein said substituted saturated alkyl is independently substituted with one or more halogen, OH, amine, CN, CF₃ or unsubstituted C₁-C₄ saturated alkyl; or R^{3H} and R^{3I}, taken together with the atom or atoms through which they are connected to form a substituted or unsubstituted 5-6 membered heterocycloalkyl or substituted or unsubstituted 5-6 membered heteroaryl, wherein the substituted 5-6 membered heterocycloalkyl or substituted 5-6 membered heteroaryl is independently substituted with one or more halogen, OH, amine, CN, CF₃, or unsubstituted C₁-C₄ saturated alkyl, wherein said amine substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy;
R⁴ is independently halogen, —OH, —CN, —CF₃, —C(O)NH₂, —C(O)OR^{4J}, —O—P(O)(OH)₂, —N^{4A}R^{4B}, —C(O)R^{4C}, —NR^{4A}C(O)R^{4C}, —NR^{4A}SO₂R^{4D}, unsubstituted C₂-C₆ saturated alkyl-NR^{4E}— unsubstituted C₂-C₆ saturated alkyl, N(R^{4H})SO₂(unsubstituted C₁-C₆ saturated alkyl)-N(R^{4H})(R^{4I}), N(R^{4H})CO(unsubstituted C₁-C₆ saturated alkyl)-N(R^{4H}) R^{4I}), unsubstituted C₁-C₆ saturated alkyl, unsubstituted C₂-C₆ alkenyl, unsubstituted C₂-C₆ alkynyl, unsubstituted C₁-C₆ saturated alkylthio, or unsubstituted C₁-C₆ cycloalkyl;
R^{4A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy
R^{4B}, R^{4C}, and R^{4D} are independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R⁴¹-substituted or unsubstituted phenyl, R⁴¹-substituted or unsubstituted heteroaryl, R⁴¹-substituted or unsubstituted heterocycloalkyl, or R⁴¹-substituted or unsubstituted heteroaryl; wherein said R⁴¹-substituted or unsubstituted phenyl, R⁴¹-substituted or unsubstituted heteroaryl, R⁴¹-substituted or unsubstituted heterocycloalkyl, and R⁴¹-substituted or unsubstituted heteroaryl are optionally substituted with one or more R⁴¹ substituents;
R⁴¹ is independently halogen, CN, CF₃, unsubstituted C₁-C₄ saturated alkyl, halo-substituted (saturated C₁-C₄ alkyl), unsubstituted C₂-C₄ alkenyl, unsubstituted C₂-C₄ alkynyl, or unsubstituted C₃-C₆ cycloalkyl;
R⁴¹ is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, substituted or unsubstituted aryl, substituted or unsubstituted heteroaryl, or substituted or unsubstituted heterocycloalkyl, wherein said substituted aryl, substituted heteroaryl, and substituted heterocyclic groups are independently substituted with one or more halogen, CN, CF₃, or unsubstituted C₁-C₄ saturated alkyl;
R^{4E} is independently hydrogen, —C(O)R^{4F}, —C(O)OR^{4F}, —SOR^{4F}, —SO₂R^{4F}, —C(O)NR^{4F}R^{4G}, or —SO₂NR^{4F}R^{4G};
R^{4G} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy;
R^{4F} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R⁴¹-substituted or unsubstituted phenyl, R⁴¹-substituted or unsubstituted heteroaryl, R⁴¹-substituted or unsubstituted heterocycloalkyl, or R⁴-substituted or unsubstituted heteroaryl; wherein said R⁴¹-substituted or unsubstituted phenyl, R⁴¹-substituted or unsubstituted heteroaryl, R⁴¹-substituted or unsubstituted heterocycloalkyl, and R⁴¹-substituted or unsubstituted heteroaryl are optionally substituted with one or more R⁴¹ substituents;
R^{4H} and R^{4I} are independently hydrogen, substituted or unsubstituted C₁-C₆ saturated alkyl, wherein said substituted saturated alkyl is independently substituted with one or more halogen, OH, amine, CN, CF₃, or unsubstituted C₁-C₄ saturated alkyl; or R^{4H} and R^{4I}, taken together with the atom or atoms through which they are connected to form a substituted or unsubstituted 5-6 membered heterocycloalkyl or substituted or unsubstituted 5-6 membered heteroaryl, wherein the substituted 5-6 membered heterocycloalkyl or substituted 5-6 membered heteroaryl is independently substituted with one or more halogen, OH, amine, CN, CF₃, or unsubstituted C₁-C₄ saturated alkyl, wherein said amine substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy;
L is halo-substituted (C₁-C₁₀ saturated alkylene), R^{L}-substituted or unsubstituted C₁-C₁₀ alkylene, R^{L}-substituted or unsubstituted C₂-C₁₀ alkenylene, R^{L}-substituted or unsubstituted C₂-C₁₀ alkynylene, R^{L}-substituted or unsubstituted C₂-C₆ saturated alkylene-O—C₂-C₆ saturated alkylene, R^{L}-substituted or unsubstituted C₂ saturated alkylene-O—C₂ saturated alkylene, R^{L}-substituted or unsubstituted C₂ saturated alkylene-O—C₂ saturated alkylene-O—C₂ saturated alkylene, R^{L}-substituted or unsubstituted C₂-C₆ saturated alkylene-NR^{L4}—C₂-C₆ saturated alkylene, R^{L}-substituted or unsubstituted C₃-C₆ cycloalkylene, R^{L}-substituted or unsubstituted phenylene, R^{L}-substituted or unsubstituted 4-6 membered heterocycloalkylene, R^{L}-substituted or unsubstituted 5-6 membered heteroarylene, R^{L}-substituted or unsubstituted C₁-C₄ saturated alkylene-(C₃-C₆ cycloalkylene)-C₁-C₄ saturated alkylene, R^{L}-substituted or unsubstituted C₁-C₄ saturated alkylene-phenylene-C₁-C₄ saturated alkylene, R^{L}-substituted or unsubstituted C₁-C₄ saturated alkylene-(4-6 membered heterocycloalkylene)-C₁-C₄ saturated alkylene, or R^{L}-substituted or unsubstituted C₁-C₄ saturated alkylene-(5-6 membered heteroarylene)-C₁-C₄ saturated alkylene;
wherein said R^{L}-substituted C₁-C₁₀ alkylene, R^{L}-substituted C₂-C₁₀ alkenylene, R^{L}-substituted C₂-C₁₀ alkynylene, R^{L}-substituted C₂-C₆ saturated alkylene-O—C₂-C₆ saturated alkylene, R^{L}-substituted C₂ saturated alkylene-O—C₂ saturated alkylene, R^{L}-substituted C₂ saturated alkylene-O—C₂ saturated alkylene-O—C₂ saturated alkylene, R^{L}-substituted C₂-C₆ saturated alkylene-NR^{L4}—C₂-C₆ saturated alkylene, R^{L}-substituted C₃-C₆ cycloalkylene, R^{L}-substituted phenylene, R^{L}-substituted 4-6 membered heterocycloalkylene, R^{L}-substituted 5-6 membered heteroarylene, R^{L}-substituted C₁-C₄ saturated alkylene-(C₃-C₆ cycloalkylene)-C₁-C₄ saturated alkylene, R^{L}-substituted C₁-C₄ saturated alkylene-phenylene-C₁-C₄ saturated alkylene, R^{L}-substituted C₁-C₄ saturated alkylene-(4-6 membered heterocycloalkylene)-C₁-C₄ saturated alkylene, and R^{L}-substituted C₁-C₄ saturated alkylene-(5-6 membered heteroarylene)-C₁-C₄ saturated alkylene are independently substituted with 1 or 2 R^{L};
R^{L} is independently halogen, —OR^{LB}, —O—P(O)(OR^{LA})₂, —NR^{LA}R^{LB}, —OC(O)R^{LB}, —C(O)—OR^{LB}, —SOR^{LB}, —SO₂R^{LB}, —C(O)NR^{LA}R^{LB}, —SO₂NR^{L A}R^{LB}, —OC(O)NR^{LA}R^{LB}, —NR^{LA}C(O)R^{LB}, —NR^{LA}SOR^{LB}, —NR^{LA}C(O)OR^{LB}, —NR^{LA}SO₂R^{LB}, —R^{LA}S O₂R^{LB}, halo-substituted (saturated C₁-C₄alkyl), (unsubstituted C₁-C₄ saturated alkyl)(unsubstituted C₁-C₄ saturated alkyl)amino, unsubstituted C₁-C₄ saturated alkyl, hydroxy-substituted (C₁-C₄ saturated alkyl), (unsubstituted C₁-C₄ saturated alkyl)-O—P(O)(OH)₂, (unsubstituted C₁-C₄ saturated alkyl)O—P(O)OR^{LA})₂, halo-substituted (C₁-C₄ alkoxy), unsubstituted C₁-C₄ alkoxy, unsubstituted C₁-C₆ saturated alkylthio, hydroxy-substituted (C₂-C₄ alkoxy), (unsubstituted C₂-C₄ alkoxy)-O—P(O)(OH)₂, (unsubstituted C₂-C₄alkoxy)-O—P(O)(OR^{LA})₂, unsubstituted C₁-C₄ alkoxy(unsubstituted C₁-C₄ alkoxy), COR^{LA}, or CON(R^{LA})(R^{LC});
R^{LA} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy
R^{LB} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R^{L6}-substituted or unsubstituted phenyl, R^{L6}-substituted or unsubstituted heteroaryl, R^{L6}-substituted or unsubstituted heterocycloalkyl, or R^{L}-substituted or unsubstituted heteroaryl; wherein said R^{L6}-substituted or unsubstituted phenyl, R^{L6}-substituted or unsubstituted heteroaryl, R^{L6}-substituted or unsubstituted heterocycloalkyl, and R^{L6}-substituted or unsubstituted heteroaryl are optionally substituted with one or more R^{L6} substituents;
R^{L6} is independently halogen, CN, CF₃, unsubstituted C₁-C₄ saturated alkyl, halo-substituted (saturated C₁-C₄ alkyl), unsubstituted C₂-C₄ alkenyl, unsubstituted C₂-C₄ alkynyl, or unsubstituted C₃-C₆ cycloalkyl;
R^{LC} is independently hydrogen, substituted or unsubstituted C₁-C₆ saturated alkyl, wherein said substituted saturated alkyl is independently substituted with one or more halogen, OH, amine, CN, CF₃, or unsubstituted C₁-C₄ saturated alkyl;
R^{L4} is independently hydrogen, COR^{L4A}, CO₂H, CO₂R^{L4A}, SOR^{L4A}, SO₂R^{L4A}, CONH₂, CONR^{L4A}R^{L4B}, SO₂NH₂, or SO₂NR^{L4A}R^{L4B};
R^{L4B} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy;
R^{L4A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R^{L6}-substituted or unsubstituted phenyl, R^{L6}-substituted or unsubstituted heterocycloalkyl, or R^{L6}-substituted or unsubstituted heteroaryl; wherein said R^{L6}-substituted or unsubstituted phenyl, R^{L6}-substituted or unsubstituted heterocycloalkyl, and R^{L6}-substituted or unsubstituted heteroaryl are optionally substituted with one or more R^{L6} substituents;
A⁹ is independently N or CR⁹;
R⁹ is independently hydrogen, halogen, —CN, —CF₃, O—P(O)(OR^{9A})₂, —OR^{9D}, —NR^{9B}R^{9B}, —NR^{9A}R^{9B}, —OC(O)NR^{9A}R^{9B}, —OC(O)R^{9C}, —C(O)R^{9C}, —C(O)—OR^{9J}, —C(O)NR^{9A}R^{9B}, —SOR^{9D}, —SO₂NR^{9A}R^{9B}, —SO₂R^{9′}, —NR^{9A}SO₂R^{9D}, —NR^{9A}C(O)R^{9C}, N(R^{9H})SO₂(unsubstituted C₁-C₄ saturated alkyl)-N(R⁹¹)(R^{9H}), N(R^{9H})CO(unsubstituted C₁-C₄ saturated alkyl)-N(R^{9I})R^{9H}), O(unsubstituted C₁-C₄ saturated alkyl)-N(R^{9I})(R^{9H}), O(unsubstituted C₁-C₄ saturated alkyl)-OR^{9B}, NR^{9H}(unsubstituted C₁-C₄ saturated alkyl)-N(R^{9I})(R^{9H}), halo-substituted (C₁-C₁₀ saturated alkyl), halo-substituted (C₁-C₁₀ alkoxy), R⁹⁰-substituted or unsubstituted (C₁-C₁₀ saturated alkyl)oxy, R⁹⁰-substituted or unsubstituted (C₁-C₁₀ saturated alkyl)amino, R⁹⁰-substituted or unsubstituted (C₁-C₆ saturated alkyl)(C₁-C₄ saturated alkyl)amino, R⁹⁰-substituted or unsubstituted phenyl, R⁹⁰-substituted or unsubstituted 5-6 membered heterocycloalkyl, or R⁹⁰-substituted or unsubstituted 5-6 membered heteroaryl;
R^{9A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy
R^{9B}, R^{9C}, and R^{9D} are independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R⁹¹-substituted or unsubstituted phenyl, R⁹¹-substituted or unsubstituted heteroaryl, R⁹¹-substituted or unsubstituted heterocycloalkyl, or R⁹¹-substituted or unsubstituted heteroaryl; wherein said R⁹¹-substituted or unsubstituted phenyl, R⁹¹-substituted or unsubstituted heteroaryl, R⁹¹-substituted or unsubstituted heterocycloalkyl, and R⁹¹-substituted or unsubstituted heteroaryl are optionally substituted with one or more R⁹¹ substituents;
R⁹¹ is independently halogen, CN, CF₃, unsubstituted C₁-C₄ saturated alkyl, halo-substituted (saturated C₁-C₄ alkyl), unsubstituted C₂-C₄ alkenyl, unsubstituted C₂-C₄ alkynyl, or unsubstituted C₃-C₆ cycloalkyl;
R^{9J} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, substituted or unsubstituted aryl, substituted or unsubstituted heteroaryl, or substituted or unsubstituted heterocycloalkyl, wherein said substituted aryl, substituted heteroaryl, and substituted heterocyclic groups are independently substituted with one or more halogen, CN, CF₃, or unsubstituted C₁-C₄ saturated alkyl;
R^{9H} and R^{9I} are independently hydrogen, substituted or unsubstituted C₁-C₆ saturated alkyl, wherein said substituted saturated alkyl is independently substituted with one or more halogen, OH, amine, CN, CF₃, or unsubstituted C₁-C₄ saturated alkyl; or R^{9H} and R^{9I}, taken together with the atom or atoms through which they are connected to form a substituted or unsubstituted 5-6 membered heterocycloalkyl or substituted or unsubstituted 5-6 membered heteroaryl, wherein the substituted 5-6 membered heterocycloalkyl or substituted 5-6 membered heteroaryl is independently substituted with one or more halogen, OH, amine, CN, CF₃, or unsubstituted C₁-C₄ saturated alkyl, wherein said amine substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy;
wherein said R⁹⁰-substituted (C₁-C₁₀ saturated alkyl)oxy, R⁹⁰-substituted (C₁-C₁₀ saturated alkyl)amino, R⁹⁰-substituted (C₁-C₆ saturated alkyl)(C₁-C₄ saturated alkyl)amino, R⁹⁰-substituted phenyl, R⁹⁰-substituted 5-6 membered heterocycloalkyl, or R⁹⁰-substituted 5-6 membered heteroaryl is substituted with 1-4 independent R⁹⁰ and
R⁹⁰ is halogen, —OR^{90B}, —O—P(O)(OR^{90A})₂, —N^{90A}R^{90B}, —NR^{90B}R^{90B}, —C(O)—OR^{90B}, —C(O)NR^{90A}R^{90B}, —SOR^{90B}, —SO₂R^{90B}, —SO₂NR^{90A}R^{90B}, —OC(O)R^{90B}, —OC(O)NR^{90A}R^{90B}, —NR^{90A}C(O)R^{90B}, —NR^{90A}SOR^{90B}, —NR^{90A}C(O)OR^{90B}, —C(O)R^{90B}, —SO₂R^{90J}, —C(O)—OR^{90J}, —C(O)NR^{90A}R^{90H}, —C(O)R^{90A}, (unsubstituted C₁-C₄ saturated alkyl)(unsubstituted C₁-C₄ saturated alkyl)amino, unsubstituted C₁-C₄ saturated alkyl, halo-substituted (C₁-C₄ saturated alkyl), hydroxy-substituted (C₁-C₄ saturated alkyl), (unsubstituted C₁-C₄ saturated alkyl)-O—P(O)(OH)₂, (unsubstituted C₁-C₄ saturated alkyl)-O—P(O)(OR^{90A})₂, halo-substituted (C₁-C₄ alkoxy), unsubstituted C₁-C₄ alkoxy, hydroxy-substituted (C₂-C₄ alkoxy), (unsubstituted C₂-C₄ alkoxy)-O—P(O)(OH)₂, (unsubstituted C₂-C₄alkoxy)-O—P(O)(OR^{90A})₂, or unsubstituted C₁-C₄ alkoxy-(unsubstituted C₁-C₄ alkoxy);
R^{90A} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, C₁-C₁₀ amino-substituted alkyl, or C₁-C₁₀ thiol-substituted saturated alkyl, wherein said amino substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy
R^{90B} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, R⁹¹-substituted or unsubstituted phenyl, R⁹¹-substituted or unsubstituted heteroaryl, R⁹¹-substituted or unsubstituted heterocycloalkyl, or R⁹¹-substituted or unsubstituted heteroaryl; wherein said R⁹¹-substituted or unsubstituted phenyl, R⁹¹-substituted or unsubstituted heteroaryl, R⁹¹-substituted or unsubstituted heterocycloalkyl, and R⁹¹-substituted or unsubstituted heteroaryl are optionally substituted with one or more R⁹¹ substituents;
R⁹¹ is independently halogen, CN, CF₃, unsubstituted C₁-C₄ saturated alkyl, halo-substituted (saturated C₁-C₄ alkyl), unsubstituted C₂-C₄ alkenyl, unsubstituted C₂-C₄ alkynyl, or unsubstituted C₃-C₆ cycloalkyl;
R^{90J} is independently hydrogen, unsubstituted C₁-C₁₀ saturated alkyl, unsubstituted C₂-C₁₀ alkenyl, unsubstituted C₂-C₁₀ alkynyl, C₁-C₁₀ hydroxy-substituted saturated alkyl, substituted or unsubstituted aryl, substituted or unsubstituted heteroaryl, or substituted or unsubstituted heterocycloalkyl, wherein said substituted aryl, substituted heteroaryl, and substituted heterocyclic groups are independently substituted with one or more halogen, CN, CF₃, or unsubstituted C₁-C₄ saturated alkyl;
R^{90H} is independently hydrogen, substituted or unsubstituted C₁-C₆ saturated alkyl, wherein said substituted saturated alkyl is independently substituted with one or more halogen, OH, amine, CN, CF₃, or unsubstituted C₁-C₄ saturated alkyl, wherein said amine substituent is NR′R″ and R′ and R″ are independently hydrogen, unsubstituted alkyl, halo-substituted alkyl, hydroxy-substituted alkyl or unsubstituted alkoxy-substituted alkyl or wherein R′ and R″, together with the nitrogen atom to which they are attached form a heterocycloalkyl or heteroaryl optionally substituted with one or more halo, oxo, hydroxy or unsubstituted alkoxy;
or a tautomer thereof,
or a salt thereof.

5. The compound of claim 1, wherein L is —CH₂—CH═CH—CH₂—.

6. The compound of claim 1, wherein R³ and R⁴ are —CONH₂.

7. The compound of claim 1, wherein A⁹ is N.

8. The compound of claim 1, wherein A⁹ is CR⁹ and R⁹ is —OMe.

9. The compound of claim 1, wherein A⁹ is CR⁹ and R⁹ is —O(CH₂)₃N(R^{9I})(R^{9H}).

10. The compound of claim 1, having the formula:
(E)-3-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide;
(E)-3-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide;
(E)-3-(4-(5-carbamoyl-2-(1-ethyl-3-methyl-1H-pyrazole-5-carboxamido)-7-(3-hydroxypropoxy)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide;
(E)-3-(4-(5-carbamoyl-2-(1-ethyl-3-methyl-1H-pyrazole-5-carboxamido)-7-(3-morpholinopropoxy)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide;
3-(4-(5-carbamoyl-2-(1-ethyl-3-methyl-1H-pyrazole-5-carboxamido)-7-(3-morpholinopropoxy)-1H-benzo[d]imidazol-1-yl)butyl)-2-(1-ethyl-3-methyl-1H-pyrazole-5-carboxamido)-3H-imidazo[4,5-b]pyridine-6-carboxamide;
(E)-3-((E)-4-((E)-5-carbamoyl-2-((1-ethyl-3-methyl-1H-pyrazole-5-carbonyl)imino)-3-methyl-7-(3-morpholinopropoxy)-2,3-dihydro-1H-benzo[d]imidazol-1-yl)but-2-en-1-yl)-2-((1-ethyl-3-methyl-1H-pyrazole-5-carbonyl)imino)-1-methyl-2,3-dihydro-1H-imidazo[4,5-b]pyridine-6-carboxamide;
(E)-3-((E)-4-((E)-5-carbamoyl-3-ethyl-2-((1-ethyl-3-methyl-1H-pyrazole-5-carbonyl)imino)-7-(3-morpholinopropoxy)-2,3-dihydro-1H-benzo[d]imidazol-1-yl)but-2-en-1-yl)-1-ethyl-2-((1-ethyl-3-methyl-1H-pyrazole-5-carbonyl)imino)-2,3-dihydro-1H-imidazo[4,5-b]pyridine-6-carboxamide;
(E)-3-(4-(5-carbamoyl-2-(1-ethyl-3-methyl-1H-pyrazole-5-carboxamido)-7-(3-(piperazin-1-yl)propoxy)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide;
3-((E)-4-(5-carbamoyl-7-(3-(4-methylpiperazin-1-yl)propoxy)-2-((E)-pent-2-enamido)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide;
(E)-3-(4-(5-carbamoyl-2-(1-ethyl-3-methyl-1H-pyrazole-5-carboxamido)-7-(2-morpholinoethoxy)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide;
(E)-3-(4-(5-carbamoyl-2-(1,5-dimethyl-1H-pyrazole-3-carboxamido)-7-(3-morpholinopropoxy)-1H-benzo[d]imidazol-1-yl)but-2-en-1-yl)-2-(1,5-dimethyl-1H-pyrazole-3-carboxamido)-3H-imidazo[4,5-b]pyridine-6-carboxamide;
(E)-3-(4-(5-carbamoyl-2-(1-isopropyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide;
(E)-3-(4-(5-carbamoyl-2-(1-cyclopropyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide;
(E)-3-(4-(5-carbamoyl-2-(3-cyclopropyl-1-ethyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide;
(E)-N-(5-carbamoyl-1-(4-(6-carbamoyl-2-(1-ethyl-3-methyl-1H-pyrazole-5-carboxamido)-3H-imidazo[4,5-b]pyridin-3-yl)but-2-en-1-yl)-7-methoxy-1H-benzo[d]imidazol-2-yl)-3-methylisoxazole-5-carboxamide;
(E)-N-(5-carbamoyl-1-(4-(6-carbamoyl-2-(1-ethyl-3-methyl-1H-pyrazole-5-carboxamido)-3H-imidazo[4,5-b]pyridin-3-yl)but-2-en-1-yl)-7-methoxy-1H-benzo[d]imidazol-2-yl)thiazole-2-carboxamide;
(E)-3-(4-(5-carbamoyl-7-methoxy-2-(pyrimidine-2-carboxamido)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide;
(E)-3-(4-(5-carbamoyl-2-(3-cyclopropyl-1-ethyl-1H-pyrazole-5-carboxamido)-7-(3-morpholinopropoxy)-1H-benzo[d]imidazol-1-yl)but-2-en-1-yl)-2-(3-cyclopropyl-1-ethyl-1H-pyrazole-5-carboxamido)-3H-imidazo[4,5-b]pyridine-6-carboxamide;
(E)-3-(4-(5-carbamoyl-2-(1,3-diethyl-1H-pyrazole-5-carboxamido)-7-(3-morpholinopropoxy)-1H-benzo[d]imidazol-1-yl)but-2-en-1-yl)-2-(1,3-diethyl-1H-pyrazole-5-carboxamido)-3H-imidazo[4,5-b]pyridine-6-carboxamide;
(E)-3-(4-(5-carbamoyl-2-(1-isopropyl-3-methyl-1H-pyrazole-5-carboxamido)-7-(3-morpholinopropoxy)-1H-benzo[d]imidazol-1-yl)but-2-en-1-yl)-2-(1-isopropyl-3-methyl-1H-pyrazole-5-carboxamido)-3H-imidazo[4,5-b]pyridine-6-carboxamide;
(E)-3-(4-(5-carbamoyl-2-(1-(cyclopropylmethyl)-3-methyl-1H-pyrazole-5-carboxamido)-7-(3-morpholinopropoxy)-1H-benzo[d]imidazol-1-yl)but-2-en-1-yl)-2-(1-(cyclopropylmethyl)-3-methyl-1H-pyrazole-5-carboxamido)-3H-imidazo[4,5-b]pyridine-6-carboxamide;
(E)-3-(4-(5-carbamoyl-2-(1-cyclopropyl-3-methyl-1H-pyrazole-5-carboxamido)-7-(3-morpholinopropoxy)-1H-benzo[d]imidazol-1-yl)but-2-en-1-yl)-2-(1-cyclopropyl-3-methyl-1H-pyrazole-5-carboxamido)-3H-imidazo[4,5-b]pyridine-6-carboxamide;
(E)-3-(4-(5-carbamoyl-2-(3-methyl-1H-pyrazole-5-carboxamido)-7-(3-morpholinopropoxy)-1H-benzo[d]imidazol-1-yl)but-2-en-1-yl)-2-(3-methyl-1H-pyrazole-5-carboxamido)-3H-imidazo[4,5-b]pyridine-6-carboxamide;
(E)-3-(4-(5-carbamoyl-2-(3-cyclopropyl-1H-pyrazole-5-carboxamido)-7-(3-morpholinopropoxy)-1H-benzo[d]imidazol-1-yl)but-2-en-1-yl)-2-(3-cyclopropyl-1H-pyrazole-5-carboxamido)-3H-imidazo[4,5-b]pyridine-6-carboxamide;
(E)-3-(4-(5-carbamoyl-2-(1-ethyl-1H-pyrrole-2-carboxamido)-7-(3-morpholinopropoxy)-1H-benzo[d]imidazol-1-yl)but-2-en-1-yl)-2-(1-ethyl-1H-pyrrole-2-carboxamido)-3H-imidazo[4,5-b]pyridine-6-carboxamide;
(E)-3-(4-(5-carbamoyl-2-(1-ethyl-1H-imidazole-2-carboxamido)-7-(3-morpholinopropoxy)-1H-benzo[d]imidazol-1-yl)but-2-en-1-yl)-2-(1-ethyl-1H-imidazole-2-carboxamido)-3H-imidazo[4,5-b]pyridine-6-carboxamide;
(E)-3-(4-(5-carbamoyl-2-(1-ethyl-5-methyl-1H-imidazole-2-carboxamido)-7-(3-morpholinopropoxy)-1H-benzo[d]imidazol-1-yl)but-2-en-1-yl)-2-(1-ethyl-5-methyl-1H-imidazole-2-carboxamido)-3H-imidazo[4,5-b]pyridine-6-carboxamide;
(E)-3-(4-(5-carbamoyl-2-(4-ethyl-5-methyl-4H-1,2,4-triazole-3-carboxamido)-7-(3-morpholinopropoxy)-1H-benzo[d]imidazol-1-yl)but-2-en-1-yl)-2-(4-ethyl-5-methyl-4H-1,2,4-triazole-3-carboxamido)-3H-imidazo[4,5-b]pyridine-6-carboxamide;
(E)-3-(4-(5-carbamoyl-2-(4-ethyl-4H-1,2,4-triazole-3-carboxamido)-7-(3-morpholinopropoxy)-1H-benzo[d]imidazol-1-yl)but-2-en-1-yl)-2-(4-ethyl-4H-1,2,4-triazole-3-carboxamido)-3H-imidazo[4,5-b]pyridine-6-carboxamide;
(E)-N-(5-carbamoyl-1-(4-(6-carbamoyl-2-(3-methylisoxazole-5-carboxamido)-3H-imidazo[4,5-b]pyridin-3-yl)but-2-en-1-yl)-7-(3-morpholinopropoxy)-1H-benzo[d]imidazol-2-yl)-3-methylisoxazole-5-carboxamide;
(E)-N-(5-carbamoyl-1-(4-(6-carbamoyl-2-(3-cyclopropylisoxazole-5-carboxamido)-3H-imidazo[4,5-b]pyridin-3-yl)but-2-en-1-yl)-7-(3-morpholinopropoxy)-1H-benzo[d]imidazol-2-yl)-3-cyclopropylisoxazole-5-carboxamide;
(E)-N-(5-carbamoyl-1-(4-(6-carbamoyl-2-(3-methylisothiazole-5-carboxamido)-3H-imidazo[4,5-b]pyridin-3-yl)but-2-en-1-yl)-7-(3-morpholinopropoxy)-1H-benzo[d]imidazol-2-yl)-3-methylisothiazole-5-carboxamide;
(E)-N-(5-carbamoyl-1-(4-(6-carbamoyl-2-(isothiazole-5-carboxamido)-3H-imidazo[4,5-b]pyridin-3-yl)but-2-en-1-yl)-7-(3-morpholinopropoxy)-1H-benzo[d]imidazol-2-yl)isothiazole-5-carboxamide;
(E)-N-(5-carbamoyl-1-(4-(6-carbamoyl-2-(thiazole-2-carboxamido)-3H-imidazo[4,5-b]pyridin-3-yl)but-2-en-1-yl)-7-(3-morpholinopropoxy)-1H-benzo[d]imidazol-2-yl)thiazole-2-carboxamide;
(E)-N-(5-carbamoyl-1-(4-(6-carbamoyl-2-(4-methylthiazole-2-carboxamido)-3H-imidazo[4,5-b]pyridin-3-yl)but-2-en-1-yl)-7-(3-morpholinopropoxy)-1H-benzo[d]imidazol-2-yl)-4-methylthiazole-2-carboxamide;
(E)-N-(5-carbamoyl-1-(4-(6-carbamoyl-2-(5-methylthiazole-2-carboxamido)-3H-imidazo[4,5-b]pyridin-3-yl)but-2-en-1-yl)-7-(3-morpholinopropoxy)-1H-benzo[d]imidazol-2-yl)-5-methylthiazole-2-carboxamide;
(E)-N-(5-carbamoyl-1-(4-(6-carbamoyl-2-(4-methylthiazole-2-carboxamido)-3H-imidazo[4,5-b]pyridin-3-yl)but-2-en-1-yl)-7-(3-morpholinopropoxy)-1H-benzo[d]imidazol-2-yl)-4-methylthiazole-2-carboxamide;
(E)-2-benzamido-3-(4-(2-benzamido-5-carbamoyl-7-(3-morpholinopropoxy)-1H-benzo[d]imidazol-1-yl)but-2-en-1-yl)-3H-imidazo[4,5-b]pyridine-6-carboxamide;
(E)-3-(4-(5-carbamoyl-7-(3-morpholinopropoxy)-2-(picolinamido)-1H-benzo[d]imidazol-1-yl)but-2-en-1-yl)-2-(picolinamido)-3H-imidazo[4,5-b]pyridine-6-carboxamide;
(E)-3-(4-(5-carbamoyl-7-(3-morpholinopropoxy)-2-(pyrimidine-2-carboxamido)-1H-benzo[d]imidazol-1-yl)but-2-en-1-yl)-2-(pyrimidine-2-carboxamido)-3H-imidazo[4,5-b]pyridine-6-carboxamide;
(E)-3-(4-(5-carbamoyl-7-(3-morpholinopropoxy)-2-(pyrazine-2-carboxamido)-1H-benzo[d]imidazol-1-yl)but-2-en-1-yl)-2-(pyrazine-2-carboxamido)-3H-imidazo[4,5-b]pyridine-6-carboxamide;
(E)-3-(4-(5-carbamoyl-7-(3-morpholinopropoxy)-2-(pyrimidine-4-carboxamido)-1H-benzo[d]imidazol-1-yl)but-2-en-1-yl)-2-(pyrimidine-4-carboxamido)-3H-imidazo[4,5-b]pyridine-6-carboxamide;
(E)-3-(4-(5-carbamoyl-7-(3-morpholinopropoxy)-2-(pyridazine-3-carboxamido)-1H-benzo[d]imidazol-1-yl)but-2-en-1-yl)-2-(pyridazine-3-carboxamido)-3H-imidazo[4,5-b]pyridine-6-carboxamide;
(E)-3-(4-(5-carbamoyl-7-(3-morpholinopropoxy)-2-(nicotinamido)-1H-benzo[d]imidazol-1-yl)but-2-en-1-yl)-2-(nicotinamido)-3H-imidazo[4,5-b]pyridine-6-carboxamide;
(E)-3-(4-(5-carbamoyl-7-(3-morpholinopropoxy)-2-(pyrimidine-5-carboxamido)-1H-benzo[d]imidazol-1-yl)but-2-en-1-yl)-2-(pyrimidine-5-carboxamido)-3H-imidazo[4,5-b]pyridine-6-carboxamide; or
(E)-3-(4-(5-carbamoyl-7-(3-morpholinopropoxy)-2-(pyridazine-4-carboxamido)-1H-benzo[d]imidazol-1-yl)but-2-en-1-yl)-2-(pyridazine-4-carboxamido)-3H-imidazo[4,5-b]pyridine-6-carboxamide.

11. The salt of a compound, or a stereoisomer or tautomer thereof, of claim 1, wherein the salt is a pharmaceutically acceptable salt.

12. A pharmaceutical composition comprising a compound of claim 1 and at least one of a pharmaceutically acceptable carrier, a pharmaceutically acceptable excipient, and a pharmaceutically acceptable diluent.

13. The compound of claim 1 or a pharmaceutically acceptable salt, stereoisomer, or tautomer thereof; for use in the manufacture of a medicament for modulating a STING protein, or for treating or preventing a disease caused by, or associated with, STING expression, activity, and/or function, or associated with deregulation of one or more of the intracellular pathways in which a STING protein is involved.

14. The pharmaceutical composition of claim 12 for use in modulating the activity of STING adaptor protein to induce production of a type I interferon, cytokine and/or chemokine dependent on the STING adaptor protein.

15. A method of treating a disease or disorder in a human or animal in need thereof, the method comprising administering to the human or animal a therapeutically effective amount of the compound of claim 1, or pharmaceutically acceptable salt, stereoisomer, or tautomer thereof.

16. The method of claim 15, where the compound or pharmaceutically acceptable salt, stereoisomer, or tautomer thereof modulates a STING protein, treats, or prevents a disease caused by, or associated with, STING expression, activity, and/or function, or associated with deregulation of one or more of the intracellular pathways in which a STING protein is involved.

17. The method of claim 15, wherein the compound or pharmaceutically acceptable salt, stereoisomer, or tautomer thereof, alone or in combination with one or more therapeutically active agents, is administered for treating or preventing a disease or condition responsive to the modulation of STING adaptor protein in a human or animal.

18. The method of claim 15, wherein the compound or pharmaceutically acceptable salt, stereoisomer, or tautomer thereof, is administered for the treatment or prevention of a viral infection, hyperproliferative disease or cancer in a human or animal.

19. The compound of claim 1, or pharmaceutically acceptable salt, stereoisomer, or tautomer thereof, for use as adjuvant in enhancing the efficacy of a vaccine in a human or animal.

20. A method of treating a disease, syndrome, condition, or disorder, wherein said disease, syndrome, condition, or disorder is modulated by the agonism of STING, comprising administering to a subject in need thereof, a therapeutically effective amount of (a) a compound of claim 1 or a pharmaceutically acceptable salt, stereoisomer, or tautomer form thereof; and (b) an oncolytic virus or anti-cancer vaccine.
